(19) United States
(12) Patent Application Publication
(10) Pub. No.: US 2008/0009530 A1
(43) Pub. Date: Jan. 10, 2008

(54) Therapeutic Agent for Diabetes

(76) Inventors: Hidenori Abe, Osaka-shi (JP); Takeshi Wakabayashi, Osaka-shi (JP); Kentarou Rikimaru, Osaka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/666,812

(22) PCT Filed: Aug. 9, 2006
(86) PCT No.: PCT/JP06/16068

§ 371 Date: Jul. 13, 2007

(30) Foreign Application Priority Data
Aug. 10, 2005 (JP) 2005-232646

(51) Int. Cl. A61K 31/415; A61K 31/44; A61P 3/10; C07D 213/02; C07D 231/06

(52) U.S. Cl. 514/351; 514/406; 546/300; 548/379.4

(57) ABSTRACT

The present invention provides an agent for the prophylaxis or treatment of diabetes, which is associated with a ferwer side effects such as body weight gain, adipocyte accumulation, cardiac hypertrophy and the like, and which contains a compound represented by the formula: wherein each symbol is as defined in the specification, or a salt thereof or a prodrug thereof.

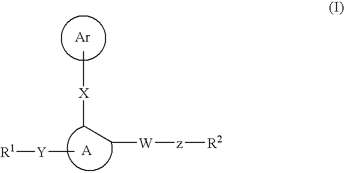

### TECHNICAL FIELD

The present invention relates to aromatic compounds useful as therapeutic agents for diabetes.

### BACKGROUND ART

As aromatic compounds, the compounds described in the following literatures are known.

(1) As a therapeutic agent for pain and the like, patent reference 1 (WO2003/016254) describes a compound represented by the formula: 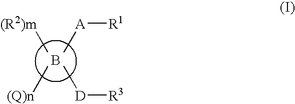
wherein R¹ is COOH, COOR⁴ (R⁴ is alkyl etc.) and the like; A is alkylene and the like; R² is alkyl and the like; m is 0, 1 or 2; B is a benzene ring and the like; Q is alkylene-Cyc2 (Cyc2 is heterocycle etc.), an alkylene-O-benzene ring and, the like; n is 0, 1 or 2; D is O-alkylene, NHCO-alkylene and the like; and R³ is a benzene ring, a naphthalene ring and the like, which has a prostaglandin E2 receptor antagonistic action.
(2) As a therapeutic agent for pain, diabetic retinopathy and the like, patent reference 2 (WO99/47497) describes a compound represented by the formula: 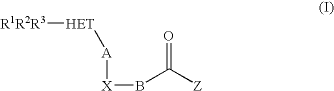
wherein HET is a 5- to 12-membered monocyclic or bicyclic aromatic ring; R¹, R² and R³ are each independently H, halogen, lower alkyl and the like; A is O, S(O)n and the like; B is —(C(R¹⁸)₂)p-Y—(C(R¹⁸)₂)q- (p and q are each independently 0-3); X is a 5- to 10-membered monocyclic aryl group or a heteroaryl group or a bicyclic aryl group or a heteroaryl group having 1-3 hetero atoms selected from O, S(O)n and N(O)m, which is substituted by R¹⁴ and R¹⁵ as necessary, (A and B are bonded to the aryl group or heteroaryl-group, and are in an ortho position with each other); Y is O, S(O)n, NR¹⁷, a bond or —CR¹⁸═CR¹⁸—; Z is OH or NHSO₂R¹⁹; R¹⁷ are each independently H, lower alkyl or Bn; R¹⁸ are each independently H, F, lower alkyl and the like; R¹⁹ is lower alkyl, lower alkenyl, lower alkynyl, CF₃, HET(R^{a})₄₋₉, lower alkyl-HET (R^{a})₄₋₉ or lower alkynyl-HET (R^{a})₄₋₉; and R^{a} is H, OH, halogen, CN, NO₂, amino, C₁₋₆ alkyl, C₁₋₆ alkenyl, C₁₋₆ alkynyl, C₁₋₆ alkoxy and the like, which is a prostaglandin receptor ligand.
(3) As a therapeutic agent for thrombosis, asthma and the like, patent reference 4 (EP-A-562796) describes a compound represented by the formula: 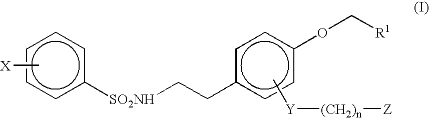
wherein X is a hydrogen atom, a lower alkyl group or a halogen atom; R¹ is a carboxyl group or a lower alkoxycarbonyl group; Y is an oxygen atom, 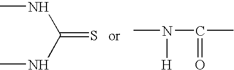
; n is an integer of 0-5; Z is 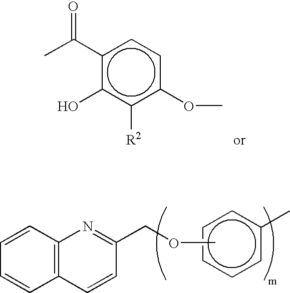
; R² is a hydrogen atom or a lower alkyl group; and m is 0 or 1, which is a thromboxane A2-antagonist and a leukotriene antagonist.
(4) As a starting material for a polymer useful as a developing solution (developing agent), patent reference 5 (US-A-2004/137380) describes a compound represented by the formula: 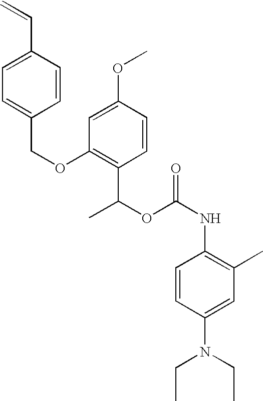
(5) As a therapeutic agent for inflammation, patent reference 6 (U.S. Pat. No. 5,597,833) describes a compound represented by the formula: 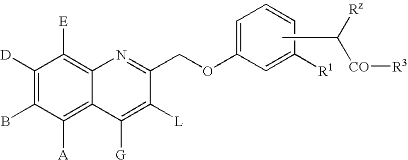
wherein A, B, D, E, G and L are each independently H and the like; R¹ is halogen, not more than C8 alkyl, alkenyl and the like (these are substituted by phenyl etc. as necessary); R² is H and the like; R³ is OH, NR⁴SO₂R⁵ and the like; R⁴ is H and the like; R⁵ is CF₃, phenyl and the like, which has a 5-lipoxygenase inhibitory action.
(6) As an intermediate for isoquinoline synthesis, non-patent reference 1 (Perkin Trans, 1, 275 (1985)) describes a compound represented by the formula: 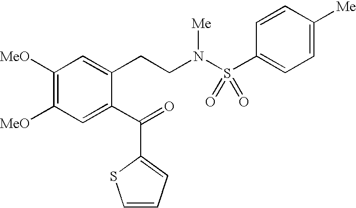
(7) Non-patent reference 2 (Archiv der Pharmazie, 316(6), 694-6 (1983)) describes a compound represented by the formula: 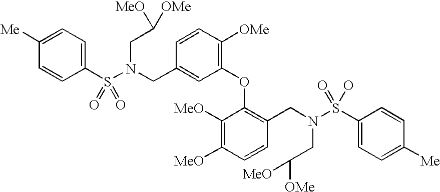
(8) Non-patent reference 3 (Arch Pharm., 141 (1964)) describes a compound represented by the formula:

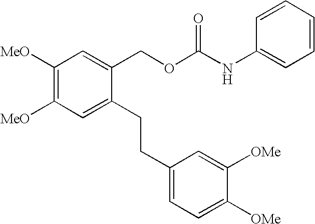

Peroxisome proliferator-activated receptor gamma (PPARγ), which is one member of the nuclear hormone receptor superfamily represented by steroid hormone receptors and thyroid gland hormone receptors, shows an induced expression at the beginning of differentiation of adipocytes and plays an important role as a master regulator in the differentiation of adipocytes. PPARγ binds to a ligand to form a dimer with retinoid X receptor (RXR), and the diner binds to a responsive element of a target gene in the nucleus to directly control (activate) the transcription efficiency.

In recent years, a possibility has been suggested that 15-deoxy-Δ12.14 prostaglandin J2, which is a metabolite of prostaglandin D2, is an endogenous ligand of PPARγ, and moreover, it has been clarified that certain insulin sensitizers represented by thiazolidinedione derivatives have a PPARγ ligand activity and the strength of the activity parallels with a hypoglycemic action or adipocyte differentiation promoting action [non-patent reference 4 (Cell, 83, 803 (1995)); non-patent reference 5 (The Journal of Biological Chemistry, 270, 12953 (1995)); non-patent reference 6 (Journal of Medicinal Chemistry, 39, 665 (1996))].

It has also been elucidated that 1) PPARγ is expressed in the cultured cell derived from human liposarcoma and the addition of PPARγ ligand stops its growth [non-patent reference 7 (Proceedings of The National Academy of Sciences of The United States of America, 94, 237 (1997))]; 2) nonsteroidal anti-inflammatory drugs represented by indomethacin and phenoprofen have a PPARγ ligand activity [non-patent reference 8 (The Journal of Biological Chemistry, 272, 3406 (1997))]; 3) PPARγ is highly expressed in activated macrophage, and the addition of its ligand leads to the inhibition of the transcription of the gene involved in inflammation [non-patent reference 9 (Nature, 391, 79 (1998))]; 4); PPARγ ligand inhibits production of inflammatory cytokines by monocyte (TNFα, IL-1β, IL-6) [non-patent reference 10 (Nature, 391, 82 (1998))] and the like.

### DISCLOSURE OF THE INVENTION

### Problems to be Solved by the Invention

There is a demand on the development of an agent for the prophylaxis or treatment of diabetes, which is associated with a fewer side effects such as body weight gain, adipocyte accumulation, cardiac hypertrophy and the like.

### Means of Solving the Problems

The present inventors have found that a compound represented by the following formula (I) has a superior hypoglycemic action, and is useful for the prophylaxis or treatment of diabetes, which resulted in the completion of the present invention. Accordingly, the present invention relates to the following.
[1] An agent for the prophylaxis or treatment of diabetes, which comprises a compound represented by the formula: 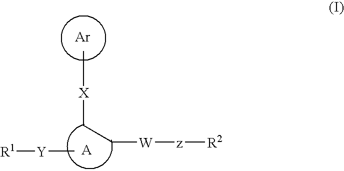
wherein ring A is an aromatic ring which is optionally further substituted;
Ar is an optionally substituted monocyclic ring;
R¹ is an optionally substituted hydrocarbon group or an optionally substituted heterocyclic group;
R² is a hydrogen atom, an optionally substituted hydrocarbon group or an optionally substituted heterocyclic group;
X is a spacer having a main chain of 1 or 2 atoms;
Y is a bond or a spacer having a main chain of 1 or 2 atoms;
W is an optionally substituted divalent hydrocarbon group having 1 to 20 carbon atoms;
Z is —CONR^{a}SO₂—, —SO₂NR^{a}CO—, —SO₂NR^{a}COO—, NR^{a}SO₂—, —OCONR^{a}SO₂—, —OCONR^{a}SO₂NR^{c}—, —OCONR^{c}—, —NR^{a}CONR^{b}SO₂, —NR^{a}SO₂NR^{b}COO— or CONR^{a}SO₂NR^{c}— (R^{a} and R^{b} are each independently a hydrogen atom, an optionally substituted hydrocarbon group or an amino-protecting group, R^{c} is a hydrogen atom, an optionally substituted hydrocarbon group or an amino-protecting group, or R^{c} and R² are bonded to each other to form, together with the adjacent nitrogen atom, an optionally substituted, nitrogen-containing heterocycle),
or a salt thereof (hereinafter sometimes to be referred to as compound (I)) or a prodrug thereof.
[2] An insulin sensitizer comprising compound (I) or a prodrug thereof.
[3] The agent of the aforementioned [1], wherein Ar is an optionally substituted monocyclic aromatic ring.
[4] A compound represented by the formula: 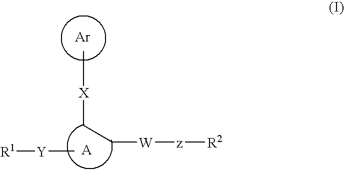
wherein ring A, Ar, R¹, R², X, Y, W, Z are as defined in the aforementioned [1] (provided that Ar is not an unsubstituted benzene ring),
or a salt thereof, which excludes the following compounds:
(4-(2-{[(4-chlorophenyl)sulfonyl]amino}-ethyl)-3-{[3-(quinolin-2-ylmethoxy)benzyl]oxy}phenoxy)acetic acid,
ethyl (4-(2-{[(4-chlorophenyl)sulfonyl]amino}ethyl)-3-{[3-(quinolin-2-ylmethoxy)benzyl]oxy}phenoxy)acetate,
1-{4-methoxy-2-[(4-vinylbenzyl)oxy]phenyl}ethyl [4-(diethylamino)-2-methylphenyl]carbamate,
N-{2-[4,5-dimethoxy-2-(2-thienylcarbonyl)phenyl]ethyl}-N,4-dimethylbenzenesulfonamide,
N-(2,2-dimethoxyethyl)-N-{3-[6-({(2,2-dimethoxyethyl)[(4-methylphenyl)sulfonyl]amino}methyl)-2,3-dimethoxyphenoxy]-4-methoxybenzyl}-4-methylbenzenesulfonamide, and
2-[2-(3,4-dimethoxyphenyl)ethyl]-4,5-dimethoxybenzyl phenylcarbamate.
   [5] The compound of the aforementioned [4], wherein Ar is an optionally substituted monocyclic aromatic ring, or a salt thereof.
   [6] The compound of the aforementioned [4], wherein Ar is an optionally substituted 5- or 6-membered monocyclic aromatic heterocycle, or a salt thereof.
   [7] The compound of the aforementioned [4], wherein Ar is a substituted benzene ring, or a salt thereof.
   [8] The compound of the aforementioned [4], wherein X is an oxygen atom, or a salt thereof.
   [9] The compound of the aforementioned [4], wherein Z is —CONR^{a}SO₂—, or a salt thereof.
   [10] The compound of the aforementioned [4], wherein R¹ is (1) a C₁₀ alkyl group or a C₂₋₁₀ alkenyl group, each optionally substituted by 1 to 3 substituents selected from a C₁₋₆ alkoxy group optionally substituted by a C₁₋₆ alkoxy group;

a carbamoyl group optionally mono- or di-substituted by a C₁₋₆ alkyl group;

an aromatic heterocyclic group optionally substituted by a C₁₋₆ alkyl group;

a non-aromatic heterocyclic group optionally substituted by 1 to 3 substituents selected from a C₁₋₆ alkyl group and an oxo group;

a C₁₋₆ alkoxy-carbonyl group;

a carboxyl group;

a hydroxy group;

a cyano group;

a silyloxy group optionally substituted by 1 to 3 substituents selected from a C₁₋₆ alkyl group and a C₆₋₁₄ aryl group;

a C₁₋₆ alkyl-carbonyloxy group;

a C₃₋₁₀ cycloalkyloxy group;

a C₃₋₁₀ cycloalkyl-C₁₋₆ alkyloxy group;

a C₁₋₆ alkylsulfonyl group;

a C₁₋₆ alkyl-carbonyl group; and

a sulfamoyloxy group;

(2) a C₃₋₁₀ cycloalkyl group;

(3) a C₆₋₁₄ aryl group;

(4) a C₇₋₁₃ aralkyl group;

(5) a C₃₋₁₀ cycloalkyl-C₁₋₆ alkyl group;

(6) a monocyclic non-aromatic heterocyclic group; or

(7) a monocyclic aromatic heterocyclic group, or a salt thereof.

[11] The compound of the aforementioned [4], wherein R² is (1) a hydrogen atom;

(2) a C₁₋₁₀ alkyl group or a C₂₋₁₀ alkenyl group, each optionally substituted by 1 to 3 substituents selected from a C₁₋₆ alkoxy group; a halogen atom; a hydroxy group; a cyano group; a C₁₋₆ alkylthio group; a carbamoyl group; a C₆₋₁₄ aryloxy group; an amino group optionally substituted by 1 or 2 substituents selected from a C₁₋₆ alkyl group, a C₁₋₆ alkyl-carbonyl group and a C₆₋₁₄ aryl group; an aromatic heterocyclic group optionally substituted by 1 to 3 C₁₋₆ alkyl group; and a non-aromatic heterocyclic group optionally substituted by 1 to 3 substituents selected from a C₁₋₆ alkyl group and an oxo group;

(3) a C₃₋₁₀ cycloalkyl group optionally substituted by a C₁₋₆ alkyl group and optionally condensed with a benzene ring;

(4) a C₆₋₁₄ aryl group optionally substituted by 1 to 3 substituents selected from a C₁₋₆ alkyl group optionally substituted by 1 to 3 halogen atoms, a hydroxy group, a C₁₋₆ alkoxy group, a halogen atom, a nitro group, and a cyano group;

(5) a C₇₋₁₃ aralkyl group optionally substituted by 1 to 3 substituents selected from a C₁₋₆ alkoxy group and a C₆₋₁₄ aryl group;

(6) a C₃₋₁₀ cycloalkyl-C₁₋₆ alkyl group; or

(7) a non-aromatic heterocyclic group optionally substituted by an oxo group, or a salt thereof.

[12] The compound of the aforementioned [4], wherein ring A is a benzene ring or a 5- or 6-membered aromatic heterocycle, or a salt thereof.

[13] The compound of the aforementioned [4], wherein Y is a bond, —O— or —SO₂—, or a salt thereof.

[14] The compound of the aforementioned [4], wherein W is C₁₋₆ alkylene or C₂₋₆ alkenylene, or a salt thereof.

[15] The compound of the aforementioned [4], which is

3-(2-{[3-chloro-5-(trifluoromethyl)pyridin-2-yl]oxy}-4-isopropoxyphenyl)-N-(pentylsulfonyl)propanamide,
(2E)-3-[2-{[3-chloro-5-(trifluoromethyl)pyridin-2-yl]oxy}-4-(2-methoxyethoxy)phenyl]-N-(pentylsulfonyl)acrylamide,
3-[2-{[3-chloro-5-(trifluoromethyl)pyridin-2-yl]oxy}-4-(2-methoxyethoxy)phenyl]propyl (pentylsulfonyl)carbamate,
3-[3-butoxy-1-(2,4-dichlorobenzyl)-1H-pyrazol-5-yl]-N-(pentylsulfonyl)propanamide,
3-{3-tert-butyl-1-[2-chloro-4-(trifluoromethyl)benzyl]-1H-pyrazol-5-yl}-N-(pentylsulfonyl)propanamide,
butyl ({2-[3-butoxy-1-(2,4-dichlorobenzyl)-1H-pyrazol-5-yl]ethyl}sulfonyl)carbamate,
(2E)-3-{2-{[3-chloro-5-(trifluoromethyl)pyridin-2-yl]oxy}-4-[2-ethoxy-1-(ethoxymethyl)ethoxy]phenyl}-N-(pentylsulfonyl)acrylamide,
3-[2-{[3-chloro-5-(trifluoromethyl)pyridin-2-yl]oxy}-4-(2-methoxyethoxy)phenyl]propyl {[(2-isopropoxyethyl)amino]sulfonyl}carbamate,
3-[2-{[3-chloro-5-(trifluoromethyl)pyridin-2-yl]oxy}-4-(2-methoxyethoxy)phenyl]propyl {[(2-pyridin-2-ylethyl)amino]sulfonyl}carbamate,
3-[2-{[3-chloro-5-(trifluoromethyl)pyridin-2-yl]oxy}-4-(2-methoxyethoxy)phenyl]-N-[(pentylamino)sulfonyl]propanamide,
(2E)-3-[2-{[3-chloro-5-(trifluoromethyl)pyridin-2-yl]oxy}-4-(2-hydroxy-2-methylpropoxy)phenyl]-N-(pentylsulfonyl)acrylamide or

3-[2-{[3-chloro-5-(trifluoromethyl)pyridin-2-yl]oxy}-4-(2-hydroxyethoxy)phenyl]propyl {[(2-isopropoxyethyl)amino]sulfonyl}carbamate, or a salt thereof.

[16] A prodrug of the compound of the aforementioned [4] or a salt thereof.

[17] A pharmaceutical agent comprising the compound of the aforementioned [4] or a salt thereof or a prodrug thereof.

[18] A method for the prophylaxis or treatment of diabetes in a mammal, which comprises administering compound (I) or a prodrug thereof to the mammal.

[19] A method of improving insulin resistance in a mammal, which comprises administering compound (I) or a prodrug thereof to the mammal.

[20] Use of compound (I) or a prodrug thereof for the production of an agent for the prophylaxis or treatment of diabetes.

[21] Use of compound (I) or a prodrug thereof for the production of an insulin sensitizer.

### EFFECT OF THE INVENTION

According to the present invention, an agent for the prophylaxis or treatment of diabetes, which is associated with a fewer side effects such as body weight gain, adipocyte accumulation, cardiac hypertrophy and the like, can be provided.

### BRIEF DESCRIPTION OF THE DRAWINGS

FIG. 1 shows an X-ray powder diffraction pattern of the crystals obtained in Example 2.

FIG. 2 shows an X-ray powder diffraction pattern of the crystals obtained in Example 12.

FIG. 3 shows an X-ray powder diffraction pattern of the crystals obtained in Example 198.

FIG. 4 shows an X-ray powder diffraction pattern of the crystals obtained in Example 204.

FIG. 5 shows an X-ray powder diffraction pattern of the crystals obtained in Example 208.

### BEST MODE FOR EMBODYING THE INVENTION

The definition of each symbol in the formula (I) is described in detail in the following.

In the present specification, the “halogen atom” is a fluorine atom, a chlorine atom, a bromine atom or an iodine atom, unless otherwise specified.

In the present specification, the “C₁₋₆ alkyl group” is methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl and the like, unless otherwise specified.

In the present specification, the “C₁₋₆ alkoxy group” is methoxy, ethoxy, propoxy, isopropoxy, butoxy, isobutoxy, sec-butoxy, tert-butoxy and the like, unless otherwise specified.

In the present specification, the “C₁₋₆ alkoxy-carbonyl group” is methoxycarbonyl, ethoxycarbonyl, propoxycarbonyl, tert-butoxycarbonyl and the like, unless otherwise specified.

In the present specification, the “C₁₋₆ alkyl-carbonyl group” is acetyl, propanoyl, butanoyl, isobutanoyl, pentanoyl, isopentanoyl, hexanoyl and the like, unless otherwise specified.

R¹ is an optionally substituted hydrocarbon group or an optionally substituted heterocyclic group.

R² is a hydrogen atom, an optionally substituted hydrocarbon group or an optionally substituted heterocyclic group.

As the “hydrocarbon group” of the “optionally substituted hydrocarbon group” for R¹ or R², for example, a C₁₋₁₀ alkyl group, a C₂₋₁₀ alkenyl group, a C₂₋₁₀ alkynyl group, a C₃₋₁₀ cycloalkyl group, a C₃₋₁₀ cycloalkenyl group, a C₄₋₁₀ cycloalkadienyl group, a C₆₋₁₄ aryl group, a C₇₋₁₃ aralkyl group, a C₈₋₁₃ arylalkenyl group, a C₃₋₁₀ cycloalkyl-C₁₋₆ alkyl group and the like can be mentioned.

As the C₁₋₁₀ alkyl group, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like can be mentioned.

As the C₂₋₁₀ alkenyl group, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like can be mentioned.

As the C₂₋₁₀ alkynyl group, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like can be mentioned.

As the C₃₋₁₀ cycloalkyl group, for example, cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like can be mentioned.

As the C₃₋₁₀ cycloalkenyl group, for example, 2-cyclopenten-1-yl, 3-cyclopenten-1-yl, 2-cyclohexen-1-yl, 3-cyclohexen-1-yl and the like can be mentioned.

As the C₄₋₁₀ cycloalkadienyl group, for example, 2,4-cyclopentadien-1-yl, 2,4-cyclohexadien-1-yl, 2,5-cyclohexadien-1-yl and the like can be mentioned.

The above-mentioned C₃₋₁₀ cycloalkyl group, C₃₋₁₀ cycloalkenyl group and C₄₋₁₀ cycloalkadienyl group each may be condensed with a benzene ring, and as such a fused ring group, for example, indanyl, dihydronaphthyl, tetrahydronaphthyl, fluorenyl and the like can be mentioned. In addition, crosslinked hydrocarbon groups such as norbornanyl, adamantly and the like can also be mentioned as the aforementioned hydrocarbon group.

As the C₆₋₁₄ aryl group, for example, phenyl, naphthyl, anthryl, phenanthryl, acenaphthylenyl, biphenylyl and the like can be mentioned.

As the C₇₋₁₃ aralkyl group, for example, benzyl, phenethyl, phenylpropyl, naphthylmethyl, biphenylylmethyl and the like can be mentioned.

As the C₈₋₁₃ arylalkenyl group, for example, styryl and the like can be mentioned.

As the C₃₋₁₀ cycloalkyl-C₁₋₆ alkyl group, for example, cyclopropylmethyl, cyclohexylmethyl and the like can be mentioned.

The C₁₋₁₀ alkyl group, C₂₋₁₀ alkenyl group and C₂₋₁₀ alkynyl group exemplified as the aforementioned “hydrocarbon group” optionally has 1 to 3 substituents at substitutable position(s).

As such substituent, for example,

(1) a C₃₋₁₀ cycloalkyl group (e.g., cyclopropyl, cyclohexyl);

(2) a C₆₋₁₄ aryl group (e.g., phenyl, naphthyl) optionally substituted by 1 to 3 substituents selected from a C₁₋₆ alkyl group optionally substituted by 1 to 3 halogen atoms, a hydroxy group, a C₁₋₆ alkoxy group, a halogen atom and a C₁₋₆ alkylsulfonyloxy group (e.g., methylsulfonyloxy);

(3) an aromatic heterocyclic group (e.g., thienyl, furyl, pyridyl, oxazolyl, thiazolyl, tetrazolyl, oxadiazolyl, pyrazinyl, quinolyl, indolyl, imidazolyl) optionally substituted by 1 to 3 substituents selected from a C₁₋₆ alkyl group optionally substituted by 1 to 3 halogen atoms, a hydroxy group, a C₁₋₆ alkoxy group and a halogen atom;

(4) a non-aromatic heterocyclic group (e.g., oxetanyl, tetrahydrofuryl, morpholinyl, thiomorpholinyl, piperidinyl, pyrrolidinyl, piperazinyl, dioxolyl, dioxolanyl, 1,3-dihydro-2-benzofuranyl, thiazolidinyl) optionally substituted by 1 to 3 substituents selected from a C₁₋₆ alkyl group optionally substituted by 1 to 3 halogen atoms, a hydroxy group, a C₁₋₆ alkoxy group, an oxo group and a halogen atom;

(5) an amino group optionally substituted by 1 or 2-substituents selected from a C₁₋₆ alkyl group, a C₁₋₆ alkyl-carbonyl group, a C₁₋₆ alkoxy-carbonyl group, a C₆₋₁₄ aryl group (e.g., phenyl), a C₆₋₁₄ aryl-carbonyl group (e.g., benzoyl), a C₇₋₁₃ aralkyl-carbonyl group (e.g., benzylcarbonyl, phenethylcarbonyl), a C₁₋₆ alkyl-aminocarbonyl group (e.g., methylaminocarbonyl, ethylaminocarbonyl), a C₆₋₁₄ aryl-aminocarbonyl group (e.g., phenylaminocarbonyl, 1-naphthylaminocarbonyl, 2-naphthylaminocarbonyl), a C₇₋₁₃ aralkyl-aminocarbonyl group (e.g., benzylaminocarbonyl), a C₁₋₆ alkylsulfonyl group (e.g., methylsulfonyl, ethylsulfonyl, isopropylsulfonyl), a C₆₋₁₄ arylsulfonyl group (e.g., benzenesulfonyl, toluenesulfonyl, 1-naphthalenesulfonyl, 2-naphthalenesulfonyl) and a C₇₋₁₃ aralkylsulfonyl group (e.g., benzylsulfonyl);

(6) an amidino group;

(7) a C₁₋₆ alkyl-carbonyl group optionally substituted by 1 to 3 halogen atoms;

(8) a C₁₋₁₆ alkoxy-carbonyl group optionally substituted by 1 to 3 halogen atoms;

(9) a C₁₋₆ alkylsulfonyl group (e.g., methylsulfonyl) optionally substituted by 1 to 3 halogen atoms;

(10) a carbamoyl group optionally mono- or di-substituted by substituent(s) selected from a C₁₋₆ alkyl group optionally substituted by 1 to 3 halogen atoms, a C₆₋₁₄ aryl group (e.g., phenyl), a C₇₋₁₃ aralkyl group (e.g., benzyl) and an aromatic heterocycle-C₁₋₆ alkyl group (e.g., furfuryl);

(11) a thiocarbamoyl group optionally mono- or di-substituted by a C₁₋₆ alkyl group optionally substituted by 1 to 3 halogen atoms;

(12) a sulfamoyl group optionally mono- or di-substituted by a C₁₋₆ alkyl group optionally substituted by 1 to 3 halogen atoms;

(13) a carboxyl group;

(14) a hydroxy group;

(15) a C₁₋₆ alkoxy group optionally substituted by 1 to 3 substituents selected from a halogen atom, a carboxyl group, a C₁₋₆ alkoxy group and a C₁₋₆ alkoxy-carbonyl group;

(16) a C₂₋₆ alkenyloxy group (e.g., ethenyloxy) optionally substituted by 1 to 3 halogen atoms;

(17) a C₃₋₁₀ cycloalkyloxy group (e.g., cyclopropyloxy, cyclohexyloxy);

(18) a C₇₋₁₃ aralkyloxy group (e.g., benzyloxy);

(19) a C₆₋₁₄ aryloxy group (e.g., phenyloxy, naphthyloxy);

(20) a C₁₋₆ alkyl-carbonyloxy group (e.g., acetyloxy, tert-butylcarbonyloxy);

(21) a mercapto group;

(22) a C₁₋₆ alkylthio group (e.g., methylthio, ethylthio) optionally substituted by 1 to 3 halogen atoms;

(23) a C₇₋₁₃ aralkylthio group (e.g., benzylthio);

(24) a C₆₋₁₄ arylthio group (e.g., phenylthio, naphthylthio);

(25) a sulfo group;

(26) a cyano group;

(27) an azido group;

(28) a nitro group;

(29) a nitroso group;

(30) a halogen atom;

(31) a C₁₋₆ alkylsulfinyl group (e.g., methylsulfinyl);

(32) an oxo group;

(33) a C₃₋₁₀ cycloalkyl-C₁₋₆ alkyloxy group (e.g., cyclopropylmethyloxy);

(34) a C₁₋₃ alkylenedioxy group (e.g., methylenedioxy, ethylenedioxy);

(35) a hydroxyimino group optionally substituted by a C₁₋₆ alkyl group;

(36) a silyloxy group optionally substituted by 1 to 3 substituents selected from a C₁₋₆ alkyl group and a C₆₋₁₄ aryl group (e.g., triisopropylsilyloxy, tert-butyl(diphenyl)silyloxy);

(37) a C₆₋₁₄ aryl-carbonyl group (e.g., benzoyl) optionally substituted by 1 to 3 halogen atoms;

(38) a sulfamoyloxy group;

(39) a carbamoyloxy group; and the like can be mentioned. When two or more substituents are used, the substituents may be the same or different.

The C₃₋₁₀ cycloalkyl group, C₃₋₁₀ cycloalkenyl group, C₄₋₁₀ cycloalkadienyl group, a C₆₋₁₄ aryl group, a C₇₋₁₃ aralkyl group, C₈₋₁₃ arylalkenyl group and C₃₋₁₀ cycloalkyl-C₁₋₆ alkyl group exemplified as the aforementioned “hydrocarbon group” optionally have 1 to 3 substituents at substitutable position(s).

As such substituent, for example,

(1) the groups exemplified as the substituents that the aforementioned C₁₋₁₀ alkyl group and the like optionally have;

(2) a C₁₋₆ alkyl group optionally substituted by 1 to 3 substituents selected from a halogen atom, a carboxyl group, a hydroxy group, a C₁₋₆ alkoxy group, a C₁₋₆ alkoxy-carbonyl group, a C₁₋₆ alkyl-carbonyloxy group (e.g., acetyloxy, tert-butylcarbonyloxy), a carbamoyl group and a non-aromatic heterocyclic group (e.g., piperidino);

(3) a C₂₋₆ alkenyl group (e.g., ethenyl, 1-propenyl) optionally substituted by 1 to 3 substituents selected from a halogen atom, a carboxyl group, a hydroxy group, a C₁₋₆ alkoxy group, a C₁₋₆ alkoxy-carbonyl group and a carbamoyl group;

(4) a C₇₋₁₃ aralkyl group (e.g., benzyl) optionally substituted by 1 to 3 substituents selected from a C₁₋₆ alkyl group optionally substituted by 1 to 3 halogen atoms, a hydroxy group, a C₁₋₆ alkoxy group and a halogen atom; and the like can be mentioned. When two or more substituents are used, the substituents may be the same or different.

As the “heterocyclic group” of the “optionally substituted heterocyclic group” for R¹ or R², an aromatic heterocyclic group and a non-aromatic heterocyclic group can be mentioned.

Here, as the aromatic heterocyclic group, for example, a 4- to 7-membered (preferably 5- or 6-membered) monocyclic aromatic heterocyclic group containing, as a ring constituting atom besides carbon atom, 1 to 4 hetero atoms selected from an oxygen atom, a sulfur atom and a nitrogen atom, and a condensed aromatic heterocyclic group can be mentioned. As the condensed aromatic heterocyclic group, for example, a group wherein such 4- to 7-membered monocyclic aromatic heterocyclic group and one or two from a 5- or 6-membered ring containing 1 or 2 nitrogen atoms, a 5-membered ring containing one sulfur atom or a benzene ring, and the like are condensed, and the like can be mentioned.

Preferable examples of the aromatic heterocyclic group include monocyclic aromatic heterocyclic groups such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl, 6-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 3-isothiazolyl, 4-isothiazolyl, 5-isothiazolyl), oxazolyl (e.g., 2-oxazolyl, 4-oxazolyl, 5-oxazolyl), isoxazolyl (e.g., 3-isoxazolyl, 4-isoxazolyl, 5-isoxazolyl), oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl), triazinyl (e.g., 1,2,4-triazin-1-yl, 1,2,4-triazin-3-yl) and the like;

condensed aromatic heterocyclic groups such as quinolyl (e.g., 2-quinolyl, 3-quinolyl, 4-quinolyl, 6-quinolyl), isoquinolyl (e.g., 3-isoquinolyl), quinazolyl (e.g., 2-quinazolyl, 4-quinazolyl), quinoxalyl (e.g., 2-quinoxalyl, 6-quinoxalyl), benzofuryl (e.g., 2-benzofuryl, 3-benzofuryl), benzothienyl (e.g., 2-benzothienyl, 3-benzothienyl), benzoxazolyl (e.g., 2-benzoxazolyl), benzisooxazolyl (e.g., 7-benzisooxazolyl), benzothiazolyl (e.g., 2-benzothiazolyl), benzimidazolyl (e.g., benzimidazol-1-yl, benzimidazol-2-yl, benzimidazol-5-yl), benzotriazolyl (e.g., 1H-1,2,3-benzotriazol-5-yl), indolyl (e.g., indol-1-yl, indol-2-yl, indol-3-yl, indol-5-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridinyl (e.g., 1H-imidazo[4,5-b]pyridin-2-yl, 1H-imidazo[4,5-c]pyridin-2-yl, 2H-imidazo[1,2-a]pyridin-3-yl), imidazopyrazinyl (e.g., 1H-imidazo[4,5-b]pyrazin-2-yl), pyrazolopyridinyl (e.g., 1H-pyrazolo[4,3-c]pyridin-3-yl), pyrazolothienyl (e.g., 2H-pyrazolo[3,4-b]thiophen-2-yl), pyrazolotriazinyl (e.g., pyrazolo[5,1-c][1,2,4]triazin-3-yl) and the like; and the like.

As the non-aromatic heterocyclic group, for example, a 4- to 7-membered (preferably 5- or 6-membered) monocyclic non-aromatic heterocyclic group containing, as a ring constituting atom besides carbon atom, 1 to 4 hetero atoms selected from an oxygen atom, a sulfur atom and a nitrogen atom, and a condensed non-aromatic heterocyclic group can be mentioned. As the condensed non-aromatic heterocyclic group, for example, a group wherein such 4- to 7-membered monocyclic non-aromatic heterocyclic group and one or two from a 5- or 6-membered ring containing 1 or 2 nitrogen atoms, a 5-membered ring containing one sulfur atom or a benzene ring, and the like are condensed, and the like can be mentioned.

As preferable examples of the non-aromatic heterocyclic group, monocyclic non-aromatic heterocyclic groups such as oxetanyl (e.g., 2-oxetanyl, 3-oxetanyl), pyrrolidinyl (e.g., 1-pyrrolidinyl, 2-pyrrolidinyl), piperidinyl (e.g., piperidino, 2-piperidinyl, 3-piperidinyl, 4-piperidinyl), morpholinyl (e.g., morpholino), thiomorpholinyl (e.g., thiomorpholino), piperazinyl (e.g., 1-piperazinyl, 2-piperazinyl, 3-piperazinyl), hexamethyleniminyl (e.g., hexamethylenimin-1-yl), oxazolidinyl (e.g., oxazolidin-2-yl), thiazolidinyl (e.g., thiazolidin-2-yl), imidazolidinyl (e.g., imidazolidin-2-yl, imidazolidin-3-yl), oxazolinyl (e.g., oxazolin-2-yl), thiazolinyl (e.g., thiazolin-2-yl), imidazolinyl (e.g., imidazolin-2-yl, imidazolin-3-yl), dioxolyl (e.g., 1,3-dioxol-4-yl), dioxolanyl (e.g., 1,3-dioxolan-4-yl), dihydrooxadiazolyl (e.g., 4,5-dihydro-1,2,4-oxadiazol-3-yl), 2-thioxo-1,3-oxazolidin-5-yl, pyranyl (e.g., 4-pyranyl), tetrahydropyranyl (e.g., 2-tetrahydropyranyl, 3-tetrahydropyranyl, 4-tetrahydropyranyl), thiopyranyl (e.g., 4-thiopyranyl), tetrahydrothiopyranyl (e.g., 2-tetrahydrothiopyranyl, 3-tetrahydrothiopyranyl, 4-tetrahydrothiopyranyl), 1-oxidotetrahydrothiopyranyl (e.g., 1-oxidotetrahydrothiopyran-4-yl), 1, 1-dioxidotetrahydrothiopyranyl (e.g., 1,1-dioxidotetrahydrothiopyran-4-yl), tetrahydrofuryl (e.g., tetrahydrofuran-3-yl, tetrahydrofuran-2-yl), pyrazolidinyl (e.g., pyrazolidin-1-yl, pyrazolidin-3-yl), pyrazolinyl (e.g., pyrazolin-1-yl), tetrahydropyrimidinyl (e.g., tetrahydropyrimidin-1-yl), dihydrotriazolyl (e.g., 2,3-dihydro-1H-1,2,3-triazol-1-yl), tetrahydrotriazolyl (e.g., 2,3,4,5-tetrahydro-1H-1,2,3-triazol-1-yl), azepanyl (e.g., azepan-3-yl) and the like;

condensed non-aromatic heterocyclic groups such as dihydroindolyl (e.g., 2,3-dihydro-1H-isoindol-1-yl), dihydroisoindolyl (e.g., 1,3-dihydro-2H-isoindol-2-yl), dihydrobenzofuranyl (e.g., 2,3-dihydro-1-benzofuran-5-yl), dihydrobenzodioxinyl (e.g., 2,3-dihydro-1,4-benzodioxinyl), dihydrobenzodioxepinyl (e.g., 3,4-dihydro-2H-1,5-benzodioxepinyl), tetrahydrobenzofuranyl (e.g., 4,5,6,7-tetrahydro-1-benzofuran-3-yl), chromenyl (e.g., 4H-chromen-2-yl, 2H-chromen-3-yl), dihydroquinolinyl (e.g., 1,2-dihydroquinolin-4-yl), tetrahydroquinolinyl (e.g., 1,2,3,4-tetrahydroquinolin-4-yl), dihydroisoquinolinyl (e.g., 1,2-dihydroisoquinolin-4-yl), tetrahydroisoquinolinyl (e.g., 1,2,3,4-tetrahydroisoquinolin-4-yl), dihydrophthalazinyl (e.g., 1,4-dihydrophthalazin-4-yl) and the like; and the like can be mentioned.

The “heterocyclic group” of the aforementioned “optionally substituted heterocyclic group” optionally has 1 to 3 substituents at substitutable position(s). As such substituent, for example, those exemplified as the substituents that the C₃₋₁₀ cycloalkyl group and the like exemplified as the “hydrocarbon group” of the aforementioned “optionally substituted hydrocarbon group” may have can be mentioned. When two or more substituents are used, the substituents may be the same or different.

R¹ is preferably

(1) a C₁₋₁₀ alkyl group (preferably methyl, ethyl, propyl, isopropyl, butyl, tert-butyl) or a C₂₋₁₀ alkenyl group (preferably 3-butenyl), each optionally substituted by 1 to 3 substituents selected from

a C₁₋₆ alkoxy group (preferably methoxy, ethoxy) optionally substituted by a C₁₋₆ alkoxy group (preferably methoxy);

a carbamoyl group optionally mono- or di-substituted by a C₁₋₆ alkyl group (preferably diethylcarbamoyl);

an aromatic heterocyclic group (preferably pyridyl, oxadiazolyl, furyl) optionally substituted by a C₁₋₆ alkyl group;

a non-aromatic heterocyclic group (preferably oxetanyl, pyrrolidinyl, tetrahydrofuryl, dioxolanyl, morpholinyl) optionally substituted by 1 to 3 substituents selected from a C₁₋₆ alkyl group and an oxo group;

a C₁₋₆ alkoxy-carbonyl group;

a carboxyl group;

a hydroxy group;

a cyano group;

a silyloxy group optionally substituted by 1 to 3 substituents selected from a C₁₋₆ alkyl group and a C₆₋₁₄ aryl group (preferably tert-butyl(diphenyl)silyloxy);

a C₁₋₆ alkyl-carbonyloxy group (preferably acetyloxy);

a C₃₋₁₀ cycloalkyloxy group (preferably cyclopropyloxy);

a C₃₋₁₀ cycloalkyl-C₁₋₆ alkyloxy group (preferably cyclopropylmethyloxy);

a C₁₋₆ alkylsulfonyl group (preferably methylsulfonyl);

a C₁₋₆ alkyl-carbonyl group;

a sulfamoyloxy group; and the like;

(2) a C₃₋₁₀ cycloalkyl group (preferably cyclopropyl);

(3) a C₆₋₁₄ aryl group (preferably phenyl);

(4) a C₇₋₁₃ aralkyl group (preferably benzyl);

(5) a C₃₋₁₀ cycloalkyl-C₁₋₆ alkyl group (preferably cyclopropylmethyl);

(6) a monocyclic non-aromatic heterocyclic group (preferably tetrahydropyranyl);

(7) a monocyclic aromatic heterocyclic group (preferably pyrimidinyl);

and the like.

R¹ is more preferably

a C₁₋₁₀ alkyl group optionally substituted by 1 to 3 substituents selected from

a C₁₋₆ alkoxy group optionally substituted by a C₁₋₆ alkoxy group; and

a hydroxy group.

R² is preferably

(1) a hydrogen atom;

(2) a C₁₋₁₀ alkyl group (preferably methyl, ethyl, propyl, isopropyl, butyl, isobutyl, tert-butyl, isobutyl, pentyl, isopentyl, neopentyl, hexyl) or a C₂₋₁₀ alkenyl group (preferably propenyl), each optionally substituted by 1 to 3 substituents selected from a C₁₋₆ alkoxy group; a halogen atom; a hydroxy group; a cyano group; a C₁₋₆ alkylthio group (preferably methylthio); a carbamoyl group; a C₆₋₁₄ aryloxy group (preferably phenyloxy); an amino group optionally substituted by 1 or 2 substituents selected from a C1.6 alkyl group, a C₁₋₆ alkyl-carbonyl group and a C₆₋₁₄ aryl group (preferably phenyl); an aromatic heterocyclic group (preferably thienyl, furyl, imidazolyl, pyridyl, pyrazinyl) optionally substituted by 1 to 3 C₁₋₆ alkyl group; and a non-aromatic heterocyclic group (preferably tetrahydrofuryl, pyrrolidinyl, morpholinyl, thiomorpholinyl) optionally substituted by 1 to 3 substituents selected from a C₁₋₆ alkyl group and an oxo group;

(3) a C₃₋₁₀ cycloalkyl group (preferably cyclohexyl, cyclobutyl, cycloheptyl, indanyl, tetrahydronaphthyl) optionally substituted by a C₁₋₆ alkyl group and optionally condensed with a benzene ring;

(4) a C₆₋₁₄ aryl group (preferably phenyl) optionally substituted by 1 to 3 substituents selected from a C₁₋₆ alkyl group optionally substituted by 1 to 3 halogen atoms, a hydroxy group, a C₁₋₆ alkoxy group, a halogen atom, a nitro group, a cyano group and the like;

(5) a C₇₋₁₃ aralkyl group (preferably benzyl, phenethyl, phenylpropyl) optionally substituted by 1 to 3 substituents selected from a C₁₋₆ alkoxy group and a C₆₋₁₄ aryl group (preferably phenyl);

(6) a C₃₋₁₀-cycloalkyl-C₁₋₆ alkyl group (preferably cyclopropylmethyl);

(7) a non-aromatic heterocyclic group (preferably azepanyl) optionally substituted by an oxo group; and the like.

R² is more preferably

a C₁₋₁₀ alkyl group (preferably methyl, ethyl, propyl, isopropyl, butyl, isobutyl, tert-butyl, isobutyl, pentyl, isopentyl, neopentyl, hexyl) optionally substituted by 1 to 3 substituents selected from a C₁₋₆ alkoxy group; a halogen atom; a hydroxy group; a cyano group; a C₁₋₆ alkylthio group (preferably methylthio); a carbamoyl group; a C₆₋₁₄ aryloxy group (preferably phenyloxy); an amino group optionally substituted by 1 or 2 substituents selected from a C₁₋₆ alkyl group, a C₁₋₆ alkyl-carbonyl group and a C₆₋₁₄ aryl group (preferably phenyl); an aromatic heterocyclic group (preferably thienyl, furyl, imidazolyl, pyridyl, pyrazinyl) optionally substituted by 1 to 3 C₁₋₆ alkyl group; and a non-aromatic heterocyclic group (preferably tetrahydrofuryl, pyrrolidinyl, morpholinyl, thiomorpholinyl) optionally substituted by 1 to 3 substituents selected from a C₁₋₆ alkyl group and an oxo group.

Ring A is an aromatic ring which is optionally further substituted. As the “aromatic ring”, for example, aromatic hydrocarbon, aromatic heterocycle and the like can be mentioned. As the aromatic hydrocarbon, for example, a C₆₋₁₄ arene and the like can be mentioned. As the C₆₋₁₄ arene, a ring constituting the C₆₋₁₄ aryl group exemplified as the aforementioned R¹ or R² can be mentioned. As the aromatic heterocycle, a ring constituting the aromatic heterocyclic group exemplified as the aforementioned R¹ or R² can be mentioned. The aromatic ring is preferably a benzene ring, a 5- or 6-membered aromatic heterocycle (preferably pyrazole, pyrrole) and the like.

The “aromatic ring” for ring A is substituted by a group —X—, a group —Y— and a group —W—, and optionally further has 1 to 3 substituents at substitutable position(s). As such substituent, those exemplified as the substituents that the C₃₋₁₀ cycloalkyl group and the like exemplified for the aforementioned R¹ or R² may have can be mentioned. When two or more substituents are used, the substituents may be the same or different. The substituent is preferably a C₁₋₆ alkyl group.

In the formula (I), the group —X— and the group —W— mean substitution at the ortho position of ring A.

Ring A is preferably a benzene ring or a 5- or 6-membered aromatic heterocycle (preferably pyrazole, pyrrole; more preferably pyrazole), each optionally substituted by a C₁₋₆ alkyl group, and the like, more preferably a benzene ring or a 5- or 6-membered aromatic heterocycle (preferably pyrazole, pyrrole; more preferably pyrazole).

When, in the formula (I), ring A is a benzene ring or a pyrazole ring, a partial structural formula

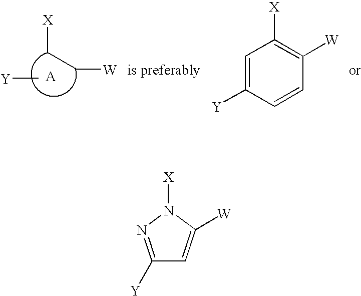

Ar is an “optionally substituted monocyclic ring”. Here, the “monocyclic ring” includes a “monocyclic aromatic ring” and a “monocyclic non-aromatic ring”. As the “monocyclic aromatic ring”, monocyclic rings from among the aromatic hydrocarbons and aromatic heterocycles exemplified as the aforementioned ring A can be mentioned. The monocyclic aromatic ring is preferably a benzene ring, a 5- or 6-membered monocyclic aromatic heterocycle (preferably pyridine, pyridazine, oxazole, thiazole) and the like. As the “monocyclic non-aromatic ring”, C₃₋₁₀ cycloalkane, C₃₋₁₀ cycloalkene and C₄₋₁₀ cycloalkadiene corresponding to the C₃₋₁₀ cycloalkyl group, C₃₋₁₀ cycloalkenyl group and C₄₋₁₀ cycloalkadienyl group exemplified as the aforementioned R¹ or R², and a monocyclic non-aromatic heterocycle corresponding to the monocyclic non-aromatic heterocyclic group exemplified as the aforementioned R¹ or R² (specifically a 5- or 6-membered monocyclic non-aromatic heterocycle containing, as a ring constituting atom besides carbon atom, 1 to 4 hetero atoms selected from an oxygen atom, a sulfur atom and a nitrogen atom; preferably piperidine, tetrahydrofuran) can be mentioned. The monocyclic non-aromatic ring is preferably a C₃₋₁₀ cycloalkane (preferably cyclopropane, cyclohexane), a 5- or 6-membered monocyclic non-aromatic heterocycle (preferably piperidine, tetrahydrofuran) and the like.

Ar is preferably an optionally substituted monocyclic aromatic ring, more preferably a “optionally substituted 5- or 6-membered monocyclic aromatic heterocycle (preferably pyridine, pyridazine, oxazole, thiazole; more preferably pyridine)” or a “substituted benzene ring”.

The monocyclic ring (monocyclic aromatic ring and monocyclic non-aromatic ring) for Ar optionally has 1 to 3 substituents at substitutable position(s). As such substituent, those exemplified as the substituents that the C₃₋₁₀ cycloalkyl group and the like exemplified as the aforementioned R¹ or R² may have can be mentioned.

The substituent of Ar is preferably

(1) a halogen atom;

(2) a C₁₋₆ alkyl group optionally substituted by 1 to 3 halogen atoms (preferably methyl, trifluoromethyl);

(3) a C₆₋₁₄ aryl group (preferably phenyl);

(4) a nitro group;

(5) a carboxyl group;

(6) a C₁₋₆ alkyl-carbonyl group (preferably acetyl);

(7) a C₁₋₆ alkoxy-carbonyl group (preferably ethoxycarbonyl, tert-butoxycarbonyl);

(8) a C₆₋₁₄ aryl-carbonyl group (preferably benzoyl);

(9) an amino group optionally substituted by 1 or 2 substituents selected from a C₁₋₆ alkyl-carbonyl group (preferably acetyl), a C₁₋₆ alkoxy-carbonyl group (preferably ethoxycarbonyl, tert-butoxycarbonyl) and C₁₋₆ alkylsulfonyl (preferably methylsulfonyl) and the like.

When the monocyclic aromatic ring for Ar is a benzene ring, Ar is preferably a substituted benzene ring.

Ar is preferably

a benzene ring, a 5- or 6-membered monocyclic aromatic heterocycle (preferably pyridine, pyridazine, oxazole, thiazole), a C₃₋₁₀ cycloalkane (preferably cyclopropane, cyclohexane) and a 5- or 6-membered monocyclic non-aromatic heterocycle (preferably piperidine, tetrahydrofuran), each optionally having 1 to 3 substituents selected from

(1) a halogen atom;

(2) a C₁₋₆ alkyl group optionally substituted by 1 to 3 halogen atoms (preferably methyl, trifluoromethyl);

(3) a C₆₋₁₄ aryl group (preferably phenyl);

(4) a nitro group;

(5) a carboxyl group;

(6) a C₁₋₆ alkyl-carbonyl group (preferably acetyl);

(7) a C₁₋₆ alkoxy-carbonyl group (preferably ethoxycarbonyl, tert-butoxycarbonyl);

(8) a C₆₋₁₄ aryl-carbonyl group (preferably benzoyl);

(9) an amino group optionally substituted by 1 or 2 substituents selected from a C₁₋₆ alkyl-carbonyl group (preferably acetyl), a C₁₋₆ alkoxy-carbonyl group (preferably ethoxycarbonyl, tert-butoxycarbonyl) and a C₁₋₆ alkylsulfonyl group (preferably methylsulfonyl); and the like.

Ar is more preferably a benzene ring or a 5- or 6-membered monocyclic aromatic heterocycle (preferably pyridine) optionally having 1 to 3 substituents selected from

(1) a halogen atom;

(2) a C₁₋₆ alkyl group optionally substituted by 1 to 3 halogen atoms (preferably methyl, trifluoromethyl); and the like.

X is a spacer having a main chain of 1 or 2 atoms.

As used herein, the “main chain” is a divalent straight chain connecting ring A and ring Ar, and the atom number of the main chain is counted so that the number of atoms in the main chain is minimum. The “main chain” consists of 1 or 2 atoms selected from a carbon atom and a hetero atom (e.g., oxygen atom, sulfur atom, nitrogen atom and the like), and may be saturated or unsaturated. The sulfur atom may be oxidized.

Y is a bond or a spacer having a main chain of 1 or 2 atoms.

As used herein, the “main chain” is a divalent straight chain connecting ring A and the group —R¹, and the atom number of the main chain is counted so that the number of atoms in the main chain is minimum. The “main chain” consists of 1 or 2 atoms selected from a carbon atom and a hetero atom (e.g., oxygen atom, sulfur atom, nitrogen atom and the like), and may be saturated or unsaturated. The sulfur atom may be oxidized.

In the “spacer having a main chain of 1 or 2 atoms” for X or Y, the carbon atom and nitrogen atom constituting the main chain optionally have one or more substituents at substitutable position(s). When two or more substituents are used, the substituents may be the same or different.

As the “substituent”, those exemplified as the substituents that the C₃₋₁₀ cycloalkyl group and the like exemplified as the aforementioned R¹ or R² may have can be mentioned.

Specific examples of the “spacer having a main chain of 1 or 2 atoms” for X or Y include

(1) —(CH₂)ₖ— wherein k is 1 or 2 (preferably —CH₂—, —CH₂CH₂—),

(2) —(CH₂)ₖ₁₁—O—(CH₂)ₖ₁₂— wherein one of k11 and k12 is 0 and the other is 0 or 1 (preferably —O—, —OCH₂—, —CH₂O—),

(3) —(CH₂)ₖ₂₁—S(O)ₖ₂₃—(CH₂)ₖ₂₂— wherein one of k21 and k22 is 0 and the other is 0 or 1, and k23 is an integer of 0 to 3 (preferably —S—, —SO₂—, —SCH₂—, —CH₂S—, —SO₂CH₂—, —CH₂SO₂—, —SO₃—),

(4) —(CH₂)ₖ₃₁—NH—(CH₂)ₖ₃₂— wherein one of k31 and k32 is 0 and the other is 0 or 1 (preferably —NH—, —NHCH₂—, —CH₂NH—) and the like, each optionally substituted by 1 to 3 substituents selected from an oxo group, a C₁₋₆ alkyl group etc.

X is preferably —(CH₂)ₖ—, —(CH₂)ₖ₁₁—O— (CH₂)ₖ₁₂—, —(CH₂)ₖ₃₁—NH— (CH₂)ₖ₃₂— and the like, each optionally substituted by an oxo group, more preferably —CH₂—, —O—, —CH₂O—, —NH—, —CO—NH— and the like, particularly preferably —CH₂— and —O—. X is particularly preferably —O— (oxygen atom).

Particularly, when Ar is an “optionally substituted 5- or 6-membered monocyclic aromatic heterocycle (preferably pyridine)”, X is preferably —O—.

When Ar is an “optionally substituted benzene ring (preferably substituted benzene ring)”, X is preferably —CH₂—.

Y is preferably a bond, —(CH₂)ₖ₁₁—O— (CH₂)ₖ₁₂—, —(CH₂) k21—S(O)ₖ₂₃—(CH₂)ₖ₂₂— and the like, more preferably a bond, —O—, —SO₂—, —SO₃— (—O—SO₂— or —SO₂—O—) and the like, particularly preferably a bond, —O— or —SO₂—. Particularly, —O— is preferable.

As the “divalent hydrocarbon group having 1 to 20 carbon atoms” for W, for example, a “divalent acyclic hydrocarbon group”, a “divalent cyclic hydrocarbon group”, or a divalent group obtained by combining one or more kinds of “divalent acyclic hydrocarbon groups” and one or more kinds of “divalent cyclic hydrocarbon groups” can be mentioned.

Here, as the “divalent acyclic hydrocarbon group”, for example, alkylene having 1 to 20 carbon atoms, alkenylene having 2 to 20 carbon atoms, alkynylene having 2 to 20 carbon atoms and the like can be mentioned.

As the “divalent cyclic hydrocarbon group”, a divalent group and the like obtained by removing any two hydrogen atoms from cycloalkane having 5 to 20 carbon atoms, cycloalkene having 5 to 20 carbon atoms or aromatic hydrocarbon having 6 to 18 carbon atoms (e.g., benzene, naphthalene, indene, anthracene) can be mentioned. Specific examples include 1,2-cyclopentylene, 1,3-cyclopentylene, 1,2-cyclohexylene, 1,3-cyclohexylene, 1,4-cyclohexylene, 1,2-cycloheptylene, 1,3-cycloheptylene, 1,4-cycloheptylene, 3-cyclohexen-1,4-ylene, 3-cyclohexen-1,2-ylene, 2,5-cyclohexadien-1,4-ylene, 1,2-phenylene, 1,3-phenylene, 1,4-phenylene, 1,4-naphthylene, 1,6-naphthylene, 2,6-naphthylene, 2,7-naphthylene, 1,5-indenylene, 2,5-indenylene and the like.

The “divalent hydrocarbon group having 1 to 20 carbon atoms” is preferably a divalent hydrocarbon group having 1 to 6 carbon atoms, and

(1) C₁₋₆ alkylene (e.g., —CH₂—, —(CH₂)₂—, —(CH₂)₃—, —(CH₂)₄—, —(CH₂)₅—, —(CH₂)₆—, —CH(CH₃)—, —CH(CH₃)CH₂—, —CH(CH₃)(CH₂)₂—, —(CH₂)₂CH(CH₃)—, —CH₂—CH(CH₃)—CH₂—, —C(CH₃)₂—, —(CH(CH₃))₂—, —(CH₂)₂C(CH₃)₂—, —(CH₂)₃C(CH₃)₂—, —CH₂—CH(CH₃)—, —CH₂—C(CH₃)₂—);

(2) C₂₋₆ alkenylene (e.g., —CH═CH—, —CH═CH—CH₂—, —CH₂—CH═CH—, —C(CH₃)₂—CH═CH—, —CH₂—CH═CH—CH₂—, —CH₂—CH₂—CH═CH—, —CH═CH—CH═CH—, —CH═CH—CH₂—CH₂—CH₂—, —CH═C(CH₃)—, —CH═C(C₂H₅)—);

(3) C₂₋₆ alkynylene (e.g., —C≡C—, —CH₂—C≡C—, —CH₂—C≡C—CH₂—CH₂—) and the like are especially preferable.

The “divalent hydrocarbon group having 1 to 20 carbon atoms” for W optionally has 1 to 3 substituents at substitutable position(s). As such substituent, for example,

(1) a halogen atom,

(2) a hydroxy group,

(3) a cyano group,

(4) a nitro group,

(5) a C₁₋₆ alkoxy group optionally substituted by 1 to 3 halogen atoms,

(6) a C₁₋₆ alkylthio group optionally substituted by 1 to 3 halogen atoms,

and the like can be mentioned.

W is preferably C₁₋₆ alkylene or C₂₋₆ alkenylene, each optionally substituted by a C₁₋₆ alkoxy group, more preferably C₁₋₆ alkylene (preferably —CH₂—, —(CH₂)₂—, —(CH₂)₃—, —CH₂—CH(CH₃)— or —CH₂—C(CH₃)₂—; more preferably —(CH₂)₂— or —(CH₂)₃—) or, C₂₋₆ alkenylene (preferably —CH═CH—, —CH═CH—CH₂—, —CH═C(CH₃)— or —CH═C(C₂H₅)—; more preferably —CH═CH—). Particularly, —(CH₂)₂—, —(CH₂)₃— and —CH═CH— are preferable.

Z is —CONR^{a}SO₂—, —SO₂NR^{a}CO—, —SO₂NR^{a}COO—, —NR^{a}SO₂—, —OCONR^{a}SO₂—, —OCONR^{a}SO₂NR^{c}—, —OCONR^{c}—, —NR^{a}CONR^{b}SO₂—, —NR^{a}SO₂NR^{b}COO— or 7CONR^{a}SO₂NR^{c}—.

Here, “R^{a}” and “R^{b}” are each independently a hydrogen atom, an optionally substituted hydrocarbon group or an amino-protecting group, “R^{c}” is a hydrogen atom, an optionally substituted hydrocarbon group or an amino-protecting group, or R^{c} and R² are bonded to each other to form, together with the adjacent nitrogen atom, an optionally substituted, nitrogen-containing heterocycle”.

As the “optionally substituted hydrocarbon group” for R^{a}, R^{b} or R^{c}C, those exemplified as the aforementioned R¹ or R² can be mentioned.

Of those, a C₁₋₆ alkyl group optionally substituted by a C₁₋₆ alkoxy group is preferable.

As the “amino-protecting group” for R^{a}, R^{b} or R^{c}, for example, formyl, a C₁₋₆ alkyl-carbonyl group, a C₁₋₆ alkoxy-carbonyl group, a benzoyl group, a C₇₋₁₀ aralkyl-carbonyl group (e.g., benzylcarbonyl), a C₇₋₁₄ aralkyloxy-carbonyl group (e.g., benzyloxycarbonyl, 9-fluorenylmethoxycarbonyl), a phthaloyl group, a substituted silyl group (e.g., a tri-C₁₋₆ alkyl-silyl group such as trimethylsilyl, triethylsilyl, tert-butyldimethylsilyl and tert-butyldiethylsilyl; tertbutyldiphenylsily) and the like can be mentioned. These groups are optionally substituted by 1 to 3 substituents selected from a halogen atom, a C₁₋₆ alkoxy group and nitro group.

As the “nitrogen-containing heterocycle” of the “optionally substituted, nitrogen-containing heterocycle” formed, together with the adjacent nitrogen atom, by R^{c} and R² bonded to each other, for example, a 5- to 7-membered nitrogen-containing heterocycle having, as a ring constituting atom besides carbon atom, at least one nitrogen atom, and further, 1 or 2 hetero atoms selected from an oxygen atom, a sulfur atom and a nitrogen atom, which is optionally condensed with a benzene ring, can be mentioned. Preferable examples of the nitrogen-containing heterocycle include pyrrolidine, imidazolidine, pyrazolidine, piperidine, piperazine, morpholine, thiomorpholine, oxopiperazine, oxopyrrolidine and dihydroisoindoline.

The nitrogen-containing heterocycle optionally have 1 to 3 (preferably 1 or 2) substituents at substitutable position(s). As such substituent, those exemplified as the substituents that the C₃₋₁₀ cycloalkyl group and the like exemplified as the aforementioned R¹ or R² may have can be mentioned. Particularly, a C₁₋₆ alkyl group optionally substituted by a C₁₋₆ alkoxy group, a carbamoyl group and the like are preferable. When two or more substituents are used, the substituents may be the same or different.

R^{a} and R^{b} are preferably each independently a hydrogen atom or a C₁₋₆ alkyl group optionally substituted by a C₁₋₆ alkoxy group, more preferably a hydrogen atom.

R^{c} is preferably a hydrogen atom or a C₁₋₆ alkyl group, or R^{c} and R² are bond to each other to form, together with the adjacent nitrogen atom, a nitrogen-containing heterocycle (preferably morpholine, pyrrolidine, piperidine, piperazine, thiomorpholine, oxopyrrolidine, dihydroisoindoline) optionally having 1 to 3 substituents selected from a C₁₋₆ alkyl group optionally substituted by a C₁₋₆ alkoxy group and a carbamoyl group.

Z is preferably —CONR^{a}SO₂—, —SO₂NR^{a}COO—, —OCONR^{a}SO₂—, —OCONR^{a}SO₂NR^{c}— or —CONR^{a}SO₂NR^{c}—, more preferably —CONR^{a}SO₂— and the like. Z is particularly preferably —CONHSO₂—.

Of compounds (I), compounds wherein Ar is not an unsubstituted benzene ring, which are other than the following compounds are novel.
(4-(2-{[(4-chlorophenyl)sulfonyl]amino}ethyl)-3-{[3-(quinolin-2-ylmethoxy)benzyl]oxy}phenoxy)acetic acid,
ethyl (4-(2-{[(4-chlorophenyl)sulfonyl]amino}ethyl)-3-{[3-(quinolin-2-ylmethoxy)benzyl]oxy}phenoxy)acetate,
1-{4-methoxy-2-[(4-vinylbenzyl)oxy]phenyl}ethyl[4-(diethylamino)-2-methylphenyl]carbamate,
N-{2-[4,5-dimethoxy-2-(2-thienylcarbonyl)phenyl]ethyl}-N, 4-dimethylbenzenesulfonamide,
N-(2,2-dimethoxyethyl)-N-{3-[6-({(2,2-dimethoxyethyl)[(4-methylphenyl)sulfonyl]amino}methyl)-2,3-dimethoxyphenoxy]-4-methoxybenzyl}-4-methylbenzenesulfonamide, and
2-[2-(3,4-dimethoxyphenyl)ethyl]-4,5-dimethoxybenzyl phenylcarbamate

Preferable examples of compound (I) include the following compounds.

[Compound AA]

Compound (I) wherein

ring A is a benzene ring, a 5- or 6-membered aromatic heterocycle etc. (preferably pyrazole, pyrrole, more preferably pyrazole), each optionally substituted by a C₁₋₆ alkyl group;

Ar is a benzene ring, a 5- or 6-membered monocyclic aromatic heterocycle (preferably pyridine, pyridazine, oxazole, thiazole), a C₃₋₁₀ cycloalkane (preferably cyclopropane, cyclohexane) and a 5- or 6-membered monocyclic non-aromatic heterocycle (preferably piperidine, tetrahydrofuran), each optionally having 1 to 3 substituents selected from

(1) a halogen atom;

(2) a C₁₋₆ alkyl group optionally substituted by 1 to 3 halogen atoms (preferably methyl, trifluoromethyl);

(3) a C₆₋₁₄ aryl group (preferably phenyl);

(4) a nitro group;

(5) a carboxyl group;

(6) a C₁₋₆ alkyl-carbonyl group (preferably acetyl);

(7) a C₁₋₆ alkoxy-carbonyl group (preferably ethoxycarbonyl, tert-butoxycarbonyl);

(8) a C₆₋₁₄ aryl-carbonyl group (preferably benzoyl);

(9) an amino group optionally substituted by 1 or 2 substituents selected from a C₁₋₆ alkyl-carbonyl group (preferably acetyl), a C₁₋₆ alkoxy-carbonyl group (preferably ethoxycarbonyl, tert-butoxycarbonyl) and a C₁₋₆ alkylsulfonyl group (preferably methylsulfonyl); and the like;

R¹ is (1) a C₁₋₁₀ alkyl group (preferably methyl, ethyl, propyl, isopropyl, butyl, tert-butyl) or a C₂₋₁₀ alkenyl group (preferably 3-butenyl), each optionally substituted by 1 to 3 substituents selected from

a C₁₋₆ alkoxy group (preferably methoxy, ethoxy) optionally substituted by a C₁₋₆ alkoxy group (preferably methoxy),

a carbamoyl group optionally mono- or di-substituted by a C₁₋₆ alkyl group (preferably diethylcarbamoyl);

an aromatic heterocyclic group (preferably pyridyl, oxadiazolyl, furyl) optionally substituted by a C₁₋₆ alkyl group;

a non-aromatic heterocyclic group (preferably oxetanyl, pyrrolidinyl, tetrahydrofuryl, dioxolanyl, morpholinyl) optionally substituted by 1 to 3 substituents selected from a C₁₋₆ alkyl group and an oxo group;

a C₁₋₆ alkoxy-carbonyl group;

a carboxyl group;

a hydroxy group;

a cyano group;

a silyloxy group optionally substituted by 1 to 3 substituents selected from a C₁₋₆ alkyl group and a C₆₋₁₄ aryl group (preferably tert-butyl(diphenyl)silyloxy);

a C₁₋₆ alkyl-carbonyloxy group (preferably acetyloxy);

a C₃₋₁₀ cycloalkyloxy group (preferably cyclopropyloxy);

a C₃₋₁₀ cycloalkyl-C₁₋₆ alkyloxy group (preferably cyclopropylmethyloxy);

a C₁₋₆ alkylsulfonyl group (preferably methylsulfonyl);

a C₁₋₆ alkyl-carbonyl group; and

a sulfamoyloxy group;

(2) a C₃₋₁₀ cycloalkyl group (preferably cyclopropyl);

(3) a C₆₋₁₄ aryl group (preferably phenyl);

(4) a C₇₋₁₃ aralkyl group (preferably benzyl);

(5) a C₃₋₁₀ cycloalkyl-C₁₋₆ alkyl group (preferably cyclopropylmethyl);

(6) a monocyclic non-aromatic heterocyclic group (preferably tetrahydropyranyl); or

(7) a monocyclic aromatic heterocyclic group (preferably pyrimidinyl);

R² iS

(1) a hydrogen atom;

(2) a C₁₋₁₀ alkyl group (preferably methyl, ethyl, propyl, isopropyl, butyl, isobutyl, tert-butyl, isobutyl, pentyl, isopentyl, neopentyl, hexyl) or a C₂₋₁₀ alkenyl group (preferably propenyl), each optionally substituted by 1 to 3 substituents selected from a C₁₋₆ alkoxy group; a halogen atom; a hydroxy group; a cyano group; a C₁₋₆ alkylthio group (preferably methylthio); a carbamoyl group; a C₆₋₁₄ aryloxy group (preferably phenyloxy); an amino group optionally substituted by 1 or 2 substituents selected from a C₁₋₆ alkyl group, a C₁₋₆ alkyl-carbonyl group and a C₆₋₁₄ aryl group (preferably phenyl); an aromatic heterocyclic group (preferably thienyl, furyl, imidazolyl, pyridyl, pyrazinyl) optionally substituted by 1 to 3 C₁₋₆ alkyl group; and a non-aromatic heterocyclic group (preferably tetrahydrofuryl, pyrrolidinyl, morpholinyl, thiomorpholinyl) optionally substituted by 1 to 3 substituents selected from a C₁₋₆ alkyl group and an oxo group;

(3) a C₃₋₁₀ cycloalkyl group (preferably cyclohexyl, cyclobutyl, cycloheptyl, indanyl, tetrahydronaphthyl) optionally substituted by a C₁₋₆ alkyl group and optionally condensed with a benzene ring;

(4) a C₆₋₁₄ aryl group (preferably phenyl) optionally substituted by 1 to 3 substituents selected from a C₁₋₆ alkyl group optionally substituted by 1 to 3 halogen atoms, a hydroxy group, a C₁₋₆ alkoxy group, a halogen atom, a nitro group, a cyano group etc.;

(5) a C₇₋₁₃ aralkyl group (preferably benzyl, phenethyl, phenylpropyl) optionally substituted by 1 to 3 substituents selected from a C₁₋₆ alkoxy group and a C₆₋₁₄ aryl group (preferably phenyl);

(6) a C₃₋₁₀ cycloalkyl-C₁₋₆ alkyl group (preferably cyclopropylmethyl); or

(7) a non-aromatic heterocyclic group (preferably azepanyl) optionally substituted by an oxo group;

X is —(CH₂)ₖ— wherein k is 1 or 2, —(CH₂)ₖ₁₁—O—(CH₂)ₖ₁₂— wherein one of k11 and k12 is 0 and the other is 0 or 1, or —(CH₂)ₖ₃₁—NH— (CH₂)ₖ₃₂— wherein one of k31 and k32 is 0 and the other is 0 or 1, each optionally substituted by an oxo group;

Y is a bond, —(CH₂)ₖ₁₁—O— (CH₂)ₖ₁₂— wherein one of k11 and k12 is 0 and the other is 0 or 1, or —(CH₂)ₖ₂₁—S(O) k23—(CH₂)ₖ₂₂— wherein one of k21 and k22 is 0 and the other is 0 or 1, k23 is an integer of 0 to 3;

W is a C₁₋₆ alkylene or a C₂₋₆ alkenylene, each optionally substituted by a C₁₋₆ alkoxy group;

Z is CONR^{a}SO₂—, —SO₂NR^{a}CO—, —SO₂NR^{a}COO—, —NR^{a}SO₂, —OCONR^{a}SO₂—, —OCONR^{a}SO₂NR^{c}—, —OCONR^{c}—, —NR^{a}CONR^{b}SO₂— or —CONR^{a}SO₂NR^{c}—;

R^{a} and R^{b} are each independently a hydrogen atom or a C₁₋₆ alkyl group optionally substituted by a C₁₋₆ alkoxy group; and

R^{c}C is a hydrogen atom or a C₁₋₆ alkyl group, or R^{c} and R² are bonded to each other to form, together with the adjacent nitrogen atom, a nitrogen-containing heterocycle (preferably morpholine, pyrrolidine, piperidine, piperazine, thiomorpholine, oxopyrrolidine, dihydroisoindoline) optionally having 1 to 3 substituents selected from a C₁₋₆ alkyl group optionally substituted by a C₁₋₆ alkoxy group and a carbamoyl group.

[Compound A]

Compound (I) wherein

ring A is a benzene ring, a 5- or 6-membered aromatic heterocycle etc. (preferably pyrazole);

Ar is a benzene ring or a 5- or 6-membered monocyclic aromatic heterocycle (preferably pyridine), each optionally having 1 to 3 substituents selected from

(1) a halogen atom;

(2) a C₁₋₆ alkyl group optionally substituted by 1 to 3 halogen atoms (preferably methyl, trifluoromethyl); and the like;

R¹ is

(1) a C₁₋₁₀ alkyl group (preferably methyl, ethyl, propyl, isopropyl, butyl, tert-butyl) optionally substituted by a substituent selected from

a C₁₋₆ alkoxy group (preferably methoxy);

a carbamoyl group (preferably diethylcarbamoyl) optionally mono-r or di-substituted by a C₁₋₆ alkyl group;

an aromatic heterocyclic group (preferably pyridyl, oxadiazolyl) optionally substituted by a C₁₋₆ alkyl group;

a non-aromatic heterocyclic group (preferably oxetanyl, pyrrolidinyl) optionally substituted by 1 to 3 substituents selected from a C₁₋₆ alkyl group and an oxo group;

a C₁₋₆ alkoxy-carbonyl group;

a carboxyl group; etc.;

(2) a C₃₋₁₀ cycloalkyl group (preferably cyclopropyl);

(3) a C₆₋₁₄ aryl group (preferably phenyl);

(4) a C₇₋₁₃ aralkyl group (preferably benzyl);

(5) a C₃₋₁₀ cycloalkyl-C₁₋₆ alkyl group (preferably cyclopropylmethyl); or

(6) a monocyclic non-aromatic heterocyclic group (preferably tetrahydropyranyl);

R² is

(1) a hydrogen atom;

(2) a C₁₋₁₀ alkyl group (preferably methyl, ethyl, propyl, isopropyl, butyl, isobutyl, tert-butyl, isobutyl, pentyl, isopentyl, neopentyl, hexyl) or a C₂₋₁₀ alkenyl group (preferably propenyl), each optionally substituted by a C₁₋₆ alkoxy group;

(3) a C₃₋₁₀ cycloalkyl group (preferably cyclohexyl);

(4) a C₆₋₁₄ aryl group (preferably phenyl) optionally substituted by 1 to 3 substituents selected from a C₁₋₆ alkyl group optionally substituted by 1 to 3 halogen atoms, a hydroxy group, a C₁₋₆ alkoxy group, a halogen atom, a nitro group etc.;

(5) a C₇₋₁₃ aralkyl group (preferably benzyl, phenylpropyl); or

(6) a C₃₋₁₀ cycloalkyl-C₁₋₆ alkyl group (preferably cyclopropylmethyl);

X is —(CH₂)ₖ— wherein k is 1 or 2 or —(CH₂)ₖ₁₁—O—(CH₂)ₖ₁₂— wherein one of k11 and k12 is 0 and the other is 0 or 1;

Y is a bond, —(CH₂)ₖ₁₁—O—(CH₂)ₖ₁₂— wherein one of k11 and k12 is 0 and the other is 0 or 1, or —(CH₂)ₖ₂₁—S(O)ₖ₂₃—(CH₂)ₖ₂₂— wherein one of k21 and k22 is 0 and the other is 0 or 1, and k23 is an integer of 0 to 3;

W is C₁₋₆ alkylene or C₂₋₆ alkenylene;

Z is —CONR^{a}SO₂—, —SO₂NR^{a}CO—, —SO₂NR^{a}COO—, —NR^{a}SO₂—OCONR^{a}SO₂—, —OCONR^{a}SO₂NR^{c}—, —OCONR^{c}—, —NR^{a}CONR^{b}SO₂— or —CONR^{a}O₂NR^{c}—;

R^{a} and R^{b} are hydrogen atoms; and

R^{c}C is a hydrogen atom or a C₁₋₆ alkyl group, or R^{c} and R² are bonded to each other to form, together with the adjacent nitrogen atom, a nitrogen-containing heterocycle (preferably morpholine).

[Compound B]

Compound (I) wherein

ring A is a benzene ring;

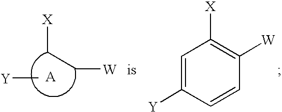

X is —(CH₂)ₖ₁₁—O— (CH₂)ₖ₁₂— wherein one of k11 and k12 is 0 and the other is 0 or 1;

Y is —(CH₂)ₖ₁₁—O— (CH₂)ₖ₁₂— wherein one of k11 and k12 is 0 and the other is 0 or 1, or —(CH₂)ₖ₂₁—S(O)ₖ₂₃—(CH₂)ₖ₂₂— wherein one of k21 and k22 is 0 and the other is 0 or 1 and k23 is an integer of 0 to 3;

Z is —CONR^{a}SO₂—, —NR^{a}SO₂—, —OCONR^{a}SO₂—, —OCONR^{a}SO₂NR^{c}—, —OCONR^{c}— or —CONR^{a}SO₂NR^{c}—;

R^{a} is a hydrogen atom;

R^{c} is a hydrogen atom or a C₁₋₆ alkyl group, or R^{c} and R² are bonded to each other to form, together with the adjacent nitrogen atom, a nitrogen-containing heterocycle (preferably morpholine); and

Ar, R¹, R² and W are as defined in the aforementioned [compound A].

[Compound C]

Compound (I) wherein

ring A is pyrazole;

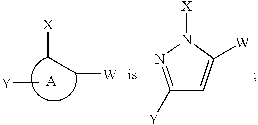

X is —(CH₂)ₖ— wherein k is 1 or 2;

Y is a bond or —(CH₂)ₖ₁₁—O— (CH₂)ₖ₁₂— wherein one of k11 and k12 is 0 and the other is 0 or 1;

Z is —CONR^{a}SO₂—, —SO₂NR^{a}CO—, —SO₂NR^{a}COO—, —OCONR^{a}SO₂— or —NR^{a}CONR^{b}SO₂—;

R^{a} and R^{b} are hydrogen atoms; and

Ar, R¹, R² and W are as defined in the aforementioned [compound A].

[Compound D]

Compound (I) which is
3-(2-{[3-chloro-5-(trifluoromethyl)pyridin-2-yl]oxy}-4-isopropoxyphenyl)-N-(pentylsulfonyl)propanamide (Example 2),
(2E)-3-[2-{[3-chloro-5-(trifluoromethyl)pyridin-2-yl]oxy}-4-(2-methoxyethoxy)phenyl]-N-(pentylsulfonyl)acrylamide (Example 12),
3-[2-{[3-chloro-5-(trifluoromethyl)pyridin-2-yl]oxy}-4-(2-methoxyethoxy)phenyl]propyl (pentylsulfonyl)carbamate (Example 28),
3-[3-butoxy-1-(2,4-dichlorobenzyl)-1H-pyrazol-5-yl]-N-(pentylsulfonyl)propanamide (Example 97),
3-{3-tert-butyl-1-[2-chloro-4-(trifluoromethyl)benzyl]-1H-pyrazol-5-yl}-N-(pentylsulfonyl)propanamide (Example 122),
butyl ({2-[3-butoxy-1-(2,4-dichlorobenzyl)-1H-pyrazol-5-yl]ethyl}sulfonyl)carbamate (Example 129),
(2E)-3-{2-{[3-chloro-5-(trifluoromethyl)pyridin-2-yl]oxy}-4-[2-ethoxy-1-(ethoxymethyl)ethoxy]phenyl}-N₇ (pentylsulfonyl)acrylamide (Example 198),
3-[2-{[3-chloro-5-(trifluoromethyl)pyridin-2-yl]oxy}-4-(2-methoxyethoxy)phenyl]propyl {[(2-isopropoxyethyl)amino]sulfonyl}carbamate (Example 204),
3-[2-{[3-chloro-5-(trifluoromethyl)pyridin-2-yl]oxy}-4-(2-methoxyethoxy)phenyl]propyl {[(2-pyridin-2-ylethyl)amino]sulfonyl}carbamate (Example 208),
3-[2-{[3-chloro-5-(trifluoromethyl)pyridin-2-yl]oxy}-4-(2-methoxyethoxy)phenyl]-N-[(pentylamino)sulfonyl]propanamide (Example 214),
(2E)-3-[2-{[3-chloro-5-(trifluoromethyl)pyridin-2-yl]oxy}-4-(2-hydroxy-2-methylpropoxy)phenyl]-N-(pentylsulfonyl)acrylamide (Example 250), or
3-[2-{[3-chloro-5-(trifluoromethyl)pyridin-2-yl]oxy}-4-(2-hydroxyethoxy)phenyl]propyl {[(2-isopropoxyethyl)amino]sulfonyl}carbamate (Example 439).

The salts with the compound represented by the formula (I) are preferably pharmacologically acceptable salts and, for example, salts with inorganic bases, salts with organic bases, salts with inorganic acids, salts with organic acids, salts with basic or acidic amino acids and the like can be mentioned.

Preferable examples of the salts with inorganic base include alkali metal salts such as sodium salt, potassium salt and the like; alkaline earth metal salts such as calcium salt, magnesium salt and the like; aluminum salt, ammonium salt and the like.

Preferable examples of the salt with organic base include a salt with trimethylamine, triethylamine, pyridine, picoline, ethanolamine, diethanolamine, triethanolamine, tromethamine[tris(hydroxymethyl)methylamine], tert-butylamine, cyclohexylamine, benzylamine, dicyclohexylamine, N,N′-dibenzylethylenediamine and the like.

Preferable examples of the salt with inorganic acid include a salt with hydrochloric acid, hydrobromic acid, nitric acid, sulfuric acid, phosphoric acid and the like.

Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of the salt with basic amino acid include a salt with arginine, lysine, ornithine and the like.

Preferable examples of the salt with acidic amino acid include a salt with aspartic acid, glutamic acid and the like.

The prodrug of the compound (I) is a compound which is converted to the compound (I) with a reaction due to an enzyme, gastric acid, etc. under the physiological condition in the living body, that is, a compound which is converted to the compound (I) by enzymatic oxidation, reduction, hydrolysis, etc.; a compound which is converted to the compound (I) by hydrolysis etc. due to gastric acid, and the like. A prodrug of the compound (I) may be a compound obtained by subjecting an amino group in the compound (I) to an acylation, alkylation or phosphorylation (e.g., a compound obtained by subjecting an amino group in the compound (I) to an eicosanoylation, alanylation, pentylaminocarbonylation, (5-methyl-2-oxo-1,3-dioxolen-4-yl)methoxycarbonylation, tetrahydrofuranylation, tetrahydropyranylation, pyrrolidylmethylation, pivaloyloxymethylation or tert-butylation); a compound obtained by subjecting a hydroxy group in the compound (I) to an acylation, alkylation, phosphorylation or boration (e.g., a compound obtained by subjecting an hydroxy group in the compound (I) to an acetylation, palmitoylation, propanoylation, pivaloylation, succinylation, fumarylation, alanylation, dimethylaminomethylcarbonylation, or tetrahydropyranylation); a compound obtained by subjecting a carboxyl group in the compound (I) to an esterification or amidation (e.g., a compound obtained by subjecting a carboxyl group in the compound (I) to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification or methylamidation) and the like. Any of these compounds can be produced from the compound (I) by a method known per se.

A prodrug of the compound (I) may be a compound that converts to the compound (I) under physiological conditions as described in Development of Pharmaceutical Products, vol. 7, Molecule Design, 163-198, Hirokawa Shoten (1990).

The compound (I) may be in the form of a crystal, and the crystal form of the crystal may be single or plural. The crystal can be produced by a crystallization method known per se. In the present specification, the melting point means that measured using, for example, a micromelting point apparatus (Yanaco, MP-500D or Buchi, B-545) or a DSC (differential scanning calorimetry) device (SEIKO, EXSTAR6000) and the like.

In general, the melting points vary depending on the measurement apparatuses, the measurement conditions and the like. The crystal in the present specification may show different values from the melting point described in the present specification, as long as they are within a general error range.

The crystal of the compound (I) is superior in physicochemical properties (melting point, solubility, stability etc.) and biological properties (pharmacokinetics (absorption, distribution, metabolism, excretion), efficacy expression, etc.), and thus it is extremely useful as a medicament.

The compound (I) may be a solvate (e.g., hydrate) or a non-solvate, both of which are encompassed in the compound (I).

A compound labeled with an isotope (e.g., ³H, ¹⁴C, ³⁵S, ¹²⁵I) and the like are also encompassed in compound (I).

The compound (I) or a prodrug thereof (hereinafter sometimes to be simply abbreviated as the compound of the present invention) shows low toxicity (e.g., acute toxicity, chronic toxicity, genetic toxicity, reproductive toxicity, cardiotoxicity, drug interaction, carcinogenicity), and can be used as it is or as a pharmaceutical composition in admixture with a commonly known pharmaceutically acceptable carrier etc., as an agent for the prophylaxis or treatment of the below-mentioned various disease, an insulin sensitizer and the like, in mammals (e.g., humans, mice, rats, rabbits, dogs, cats, bovines, horses, pigs, monkeys).

Here, as the pharmacologically acceptable carrier, various organic or inorganic carrier substances conventionally used as a preparation material can be used. They are incorporated as excipient, lubricant, binder and disintegrant for solid preparations; solvent, dissolution aids, suspending agent, isotonicity agent, buffer and soothing agent for liquid preparations and the like. Where necessary, preparation additives such as preservatives, antioxidants, coloring agents, sweetening agents and the like can be used.

As preferable examples of the excipient, lactose, sucrose, D-mannitol, D-sorbitol, starch, α-starch, dextrin, crystalline cellulose, low-substituted hydroxypropylcellulose, sodium carboxymethylcellulose, gum arabic, pullulan, light anhydrous silicic acid, synthetic aluminum silicate, magnesium alumino metasilicate and the like can be mentioned.

As preferable examples of the lubricant, magnesium stearate, calcium stearate, talc, colloidal silica and the like can be mentioned.

As preferable examples of the binder, α-starch, saccharose, gelatin, gum arabic, methylcellulose, carboxymethylcellulose, carboxymethylcellulose sodium, crystalline cellulose, sucrose, D-mannitol, trehalose, dextrin, pullulan, hydroxypropylcellulose, hydroxypropylmethylcellulose, polyvinylpyrrolidone and the like can be mentioned.

As preferable examples of the disintegrant, lactose, sucrose, starch, carboxymethylcellulose, carboxymethylcellulose calcium, croscarmellose sodium, carboxymethylstarch sodium, light anhydrous silicic acid, low-substituted hydroxypropylcellulose and the like can be mentioned.

As preferable examples of the solvent, water for injection, physiological brine, Ringer solution, alcohol, propylene glycol, polyethylene glycol, sesame oil, corn oil, olive oil, cottonseed oil and the like can be mentioned.

As preferable examples of the dissolution aids, polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like can be mentioned.

As preferable examples of the suspending agent, surfactants such as stearyltriethanolamine, sodium lauryl sulfate, laurylaminopropionic acid, lecithin, benzalkonium chloride, benzethonium chloride, glycerol monostearate and the like; hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose and the like; polysorbates, polyoxyethylene hydrogenated castor oil, and the like can be mentioned.

As preferable examples of the isotonicity agent, sodium chloride, glycerin, D-mannitol, D-sorbitol, glucose and the like can be mentioned.

As preferable examples of the buffer, buffers such as phosphate, acetate, carbonate, citrate and the like, and the like can be mentioned.

As preferable examples of the soothing agent, benzyl alcohol and the like can be mentioned.

As preferable examples of the preservative, p-oxybenzoates, chlorobutanol, benzyl alcohol, phenethyl alcohol, dehydroacetic acid, sorbic acid and the like can be mentioned.

As preferable examples of the antioxidant, sulfite, ascorbate and the like can be mentioned

As preferable examples of the coloring agent, water-soluble food tar colors (e.g., food colors such as Food Red Nos. 2 and 3, Food Yellow Nos. 4 and 5, Food Blue Nos. 1 and 2 and the like), water insoluble lake dye (e.g., aluminum salts of the aforementioned water-soluble food tar colors), natural dyes (e.g., β-carotene, chlorophyll, red iron oxide) and the like can be mentioned.

As preferable examples of the sweetening agent, saccharin sodium, dipotassium glycyrrhizinate, aspartame, stevia and the like can be mentioned.

As the dosage form of the aforementioned pharmaceutical composition, for example, oral preparation such as tablets (including sublingual tablet, orally disintegrating tablet), capsules (including soft capsule, microcapsule), granule, powder, troche, syrup, emulsion, suspension and the like; and parenteral preparation such as injections (e.g., subcutaneous injection, intravenous injection, intramuscular injection, intraperitoneal injection, drip infusion), external preparations (e.g., dermal preparation, ointment), suppositories (e.g., rectal suppository, vaginal suppository), pellets, transnasal preparations, pulmonary preparations (inhalant), eye drops and the like can be mentioned. They can be safely administered orally or parenterally.

These preparations may be controlled-release preparations (e.g., sustained-release microcapsule) such as immediate-release preparation, sustained-release preparation and the like.

The pharmaceutical composition can be produced by a method conventionally used in the preparation technical field, such as a method described in the Japanese Pharmacopoeia and the like.

While the content of the compound of the present invention in the pharmaceutical composition varies depending on the dosage form, the dose of the compound of the present invention and the like, it is, for example, about 0.1 to 100 wt %.

The compound of the present invention can be used as an insulin sensitizer, an agent for enhancing insulin sensitivity, a retinoid-related receptor function regulator, a peroxisome proliferator-activated receptor ligand, a retinoid X receptor ligand and the like. The function regulator here means both agonists and antagonists.

The compound of the present invention has a hypoglycemic action, a hypolipidemic action, a blood insulin lowering action, an insulin resistance improving action, an insulin sensitivity enhancing action and a retinoid-related receptor function regulating activity. The function regulator may be a partial agonist or partial antagonist.

Here, the retinoid-related receptors are DNA binding transcription factors included in the nuclear receptors and using a signal molecule such as fat-soluble vitamin and the like as a ligand, which may be monomer receptors, homodimer receptors or heterodimer receptors.

Here, as the monomer receptors, for example, retinoid 0 receptor (hereinafter sometimes to be abbreviated as ROR) a (GenBank Accession No. L14611), RORβ (GenBank Accession No. L14160), RORγ (GenBank Accession No. U16997); Rev-erb α (GenBank Accession No. M24898), Rev-erb β (GenBank Accession No. L31785); ERRα (GenBank Accession No. X51416), ERRβ (GenBank Accession No. X51417); Ftz-FI α (GenBank Accession No. S65876), Ftz-FI β (GenBank Accession No. M81385); TIx (GenBank Accession No. S77482); GCNF (GenBank Accession No. U14666) and the like can be mentioned.

As the homodimer receptors, for example, homodimers formed by retinoid X receptors (hereinafter sometimes to be abbreviated as RXR) a (GenBank Accession No. X52773), RXRβ (GenBank Accession No. M84820), RXRγ (GenBank Accession No. U38480); COUPα (GenBank Accession No. X12795), COUPβ (GenBank Accession No. M64497), COUPγ (GenBank Accession No. X12794); TR2α (GenBank Accession No. M29960), TR2β (GenBank Accession No. L27586); or HNF4α (GenBank Accession No. X76930), HNF4γ (GenBank Accession No. Z49826) and the like can be mentioned.

As the heterodimer receptors, for example, heterodimers formed by the above-mentioned retinoid X receptors (RXRα, RXRβ or RXRγ) and one kind of receptor selected from retinoid A receptor (hereinafter sometimes to be abbreviated as RAR) α (GenBank Accession No. X06614), RARβ (GenBank Accession No. Y00291), RARγ (GenBank Accession No. M24857); thyroid gland hormone receptor (hereinafter sometimes to be abbreviated as TR) α (GenBank Accession No. M24748), TRβ (GenBank Accession No. M26747); vitamin D receptor (VDR) (GenBank Accession No. J03258); peroxisome proliferator-activated receptor (hereinafter sometimes to be abbreviated as PPAR) α (GenBank Accession No. L02932), PPARβ (PPARδ)(GenBank Accession No. U10375), PPAR_{γ} (GenBank Accession No. L40904); LXRα ((GenBank Accession No. U22662), LXRβ (GenBank Accession No. U14534); FXR (GenBank Accession No. U18374); MB67 (GenBank Accession No. L29263); ONR (GenBank Accession No. X75163); and NURα ((GenBank Accession No. L13740), NURβ (GenBank Accession No. X75918) and NURγ (GenBank Accession No. U12767) can be mentioned.

The compound of the present invention has a superior ligand activity (activating action) for retinoid X receptors (RXRα, RXRβ, RXRγ) and peroxisome proliferator-activated receptors (PPARα, PPARβ (PPARδ), PPARγ), from among the above-mentioned retinoid-related receptors, and is useful as an agonist, a partial agonist, an antagonist or a partial antagonist of these receptors.

Moreover, the compound of the present invention has a superior ligand activity (activating action) for peroxisome proliferator-activated receptor of heterodimer receptors formed by retinoid X receptor and peroxisome proliferator-activated receptor (e.g., heterodimer receptor formed by RXRα and PPARδ, heterodimer receptor formed by RXRα and PPARγ).

Therefore, the compound of the present invention is preferably used as a peroxisome proliferator-activated receptor ligand or a retinoid X receptor ligand.

The compound of the present invention is useful as a hypoglycemic agent free of side effects such as body weight gain, adipocyte accumulation, cardiac hypertrophy and the like.

The compound of the present invention can be used, for example, as an agent for the prophylaxis or treatment of diabetes (e.g., type-1 diabetes, type-2 diabetes, gestational diabetes, obesity diabetes); an agent for the prophylaxis or treatment of hyperlipidemia (e.g., hypertriglyceridemia, hypercholesterolemia, hypo-HDL-emia, postprandial hyperlipidemia); insulin sensitizer; an agent for enhancing insulin sensitivity; an agent for the prophylaxis or treatment of impaired glucose tolerance [IGT (Impaired Glucose Tolerance)]; and an agent for preventing progress of impaired glucose tolerance into diabetes.

For diagnostic criteria of diabetes, Japan Diabetes Society reported new diagnostic criteria.

According to this report, diabetes is a condition showing any of a fasting blood glucose level (glucose concentration of intravenous plasma) of not less than 126 mg/dl, a 75 g oral glucose tolerance test (75 g OGTT) 2 h level (glucose concentration of intravenous plasma) of not less than 200 mg/dl, and a non-fasting blood glucose level (glucose concentration of intravenous plasma) of not less than 200 mg/dl. A condition not falling under the above-mentioned diabetes and different from “a condition showing a fasting blood glucose level (glucose concentration of intravenous plasma) of less than 110 mg/dl or a 75 g oral glucose tolerance test (75 g OGTT) 2 h level (glucose concentration of intravenous plasma) of less than 140 mg/dl” (normal type) is called a “borderline type”.

In addition, ADA (American Diabetes Association) and WHO reported new diagnostic criteria of diabetes.

According to these reports, diabetes is a condition showing a fasting blood glucose level (glucose concentration of intravenous plasma) of not less than 126 mg/dl and a 75 g oral glucose tolerance test 2 h level (glucose concentration of intravenous plasma) of not less than 200 mg/dl.

According to the above-mentioned reports of ADA and WHO, impaired glucose tolerance is a condition showing a 75 g oral glucose tolerance test 2 h level (glucose concentration of intravenous plasma) of not less than 140 mg/dl and less than 200 mg/dl. According to the report of ADA, a condition showing a fasting blood glucose level (glucose concentration of intravenous plasma) of not less than 100 mg/dl and less than 126 mg/dl is called IFG (Impaired Fasting Glucose). On the other hand, WHO defines the IFG (Impaired Fasting Glucose) to be a condition showing a fasting blood glucose level (glucose concentration of intravenous plasma) of not less than 110 mg/dl and less than 126 mg/dl, and calls it IFG (Impaired Fasting Glycaemia).

The compound of the present invention can be also used as an agent for the prophylaxis or treatment of diabetes, borderline type, impaired glucose tolerance, IFG (Impaired Fasting Glucose) and IFG (Impaired Fasting Glycaemia), as determined according to the above-mentioned new diagnostic criteria. Moreover, the compound of the present invention can prevent progress of borderline type, impaired glucose tolerance, IFG (Impaired Fasting Glucose) or IFG (Impaired Fasting Glycaemia) into diabetes.

The compound of the present invention can also be used as an agent for the prophylaxis or treatment of, for example, diabetic complications [e.g., neuropathy, nephropathy, retinopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, peripheral blood circulation disorder], obesity, osteoporosis, cachexia (e.g., cancerous cachexia, tuberculous cachexia, diabetic cachexia, blood disease cachexia, endocrine disease cachexia, infectious disease cachexia or cachexia due to acquired immunodeficiency syndrome), fatty liver, hypertension, polycystic ovary syndrome, kidney disease (e.g., diabetic nephropathy, glomerular nephritis, glomerulosclerosis, nephrotic syndrome, hypertensive nephrosclerosis, end stage kidney disease), muscular dystrophy, myocardial infarction, angina pectoris, cerebrovascular accident (e.g., cerebral infarction, cerebral apoplexy), insulin resistance syndrome, Syndrome X, metabolic syndrome (pathology having three or more selected from hypertriglyceridemia (TG), hypoHDL cholesterolemia (HDL-C), hypertension, abdomen overweight and impaired glucose tolerance), hyperinsulinemia, hyperinsulinemia-induced sensory disorder, tumor (e.g., leukemia, breast cancer, prostate cancer, skin cancer), irritable bowel syndrome, acute or chronic diarrhea, inflammatory diseases (e.g., arteriosclerosis (e.g., atherosclerosis), chronic rheumatoid arthritis, spondylitis deformans, osteoarthritis, lumbago, gout, postoperative or traumatic inflammation, swelling, neuralgia, pharyngolaryngitis, cystitis, hepatitis (inclusive of nonalcoholic steatohepatitis), pneumonia, pancreatitis, inflammatory bowel disease, ulcerative colitis, chronic obstructive pulmonary disease (COPD)), visceral obesity syndrome, leg ulcer, sepsis, psoriasis and the like.

In addition, the compound of the present invention can also be used for ameliorating the conditions such as abdominal pain, nausea, vomiting, discomfort in the upper abdomen and the like, which are associated with peptic ulcer, acute or chronic gastritis, biliary dyskinesia, cholecystitis and the like, and the like.

The compound of the present invention can also be used as an agent for the prophylaxis or treatment of inflammatory disease involving TNF-α. Here, the inflammatory disease involving TNF-α is an inflammatory disease developed by the presence of TNF-α, which can be treated via a TNF-(inhibitory effect. As such inflammatory disease, for example, diabetic complications (e.g., retinopathy, nephropathy, neuropathy, macroangiopathy), chronic rheumatoid arthritis, spondylitis deformans, osteoarthritis, lumbago, gout, postoperative or traumatic inflammation, swelling, neuralgia, pharyngolaryngitis, cystitis, hepatitis, pneumonia, stomach mucous membrane injury (including stomach mucous membrane injury caused by aspirin) and the like can be mentioned.

The compound of the present invention has an apoptosis inhibitory action and can also be used as an agent for the prophylaxis or treatment of diseases involving promotion of apoptosis. As the disease involving promotion of apoptosis, for example, viral diseases (e.g., AIDS, fulminant hepatitis), neurodegenerative diseases (e.g., Alzheimer's disease, Parkinson's syndrome, amyotrophic lateral sclerosis, pigmentosa, cerebellar degeneration), myelodysplasia (e.g., aplastic anemia), ischemic diseases (e.g., cardiac infarction, cerebral apoplexy), hepatic diseases (e.g., alcoholic hepatitis, hepatitis B, hepatitis C), joint-diseases (e.g., osteoarthritis), atherosclerosis and the like can be mentioned.

The compound of the present invention can also be used for reduction of visceral fat, inhibition of visceral fat accumulation, glycometabolism improvement, lipometabolism improvement, insulin resistance improvement, oxidized LDL production inhibition, lipoprotein metabolism improvement, coronary metabolism improvement, prophylaxis or treatment of cardiovascular complications, prophylaxis or treatment of heart failure complications, decrease of blood remnant, prophylaxis or treatment of anovulation, prophylaxis or treatment of hirsutism, prophylaxis or treatment of hyperandrogenemia and the like.

The compound of the present invention can also be used as secondary prevention and suppression of progression of the above-mentioned various diseases (e.g., cardiovascular event such as cardiac infarction and the like).

While the dose of the compound of the present invention varies depending on the administration subject, administration route, target disease, condition and the like, for example, it is generally about 0.005 to 50 mg/kg body weight, preferably 0.01 to 2 mg/kg body weight, more preferably 0.025 to 0.5 mg/kg body weight, for oral administration to adult diabetic patients, which is desirably administered in one to three portions a day.

The compound of the present invention can be used in combination with pharmaceutical agents (hereinafter to be abbreviated as combination drug) such as therapeutic agents for diabetes, therapeutic agents for diabetic complications, therapeutic agents for hyperlipidemia, antihypertensive agents, antiobesity agents, diuretics, chemotherapeutic agents, immunotherapeutic agents, antithrombotic agents, therapeutic agents for osteoporosis, antidementia agents, erectile dysfunction ameliorating agents, therapeutic agents for urinary incontinence or pollakiuria, therapeutic agents for dysuria and the like. These combination drugs may be low-molecular-weight compounds, or high-molecular-weight protein, polypeptide, antibody, vaccine and the like.

The administration time of the compound of the present invention and the combination drug is not restricted, and these can be administered to an administration subject simultaneously, or may be administered at staggered times.

As the administration mode of the compound of the present invention and the combination drug, the following methods can be mentioned: (1) The compound of the present invention and the combination drug are simultaneously formulated to give a single preparation which is administered. (2) The compound of the present invention and the combination drug are separately formulated to give two kinds of preparations which are administered simultaneously by the same administration route. (3) The compound of the present invention and the combination drug are separately formulated to give two kinds of preparations which are administered by the same administration route at staggered times. (4) The compound of the present invention and the combination drug are separately formulated to give two kinds of preparations which are administered simultaneously by the different administration routes. (5) The compound of the present invention and the combination drug are separately formulated to give two kinds of preparations which are administered by the different administration routes at staggered times (for example, the compound of the present invention and the combination drug are administered in this order, or in the reverse order), and the like.

The dose of the combination drug can be appropriately determined based on the dose employed clinically.

The mixing ratio of the compound of the present invention and a combination drug can be appropriately determined depending on the administration subject, administration route, target disease, symptom, combination and the like. When the administration subject is human, for example, a combination drug can be used in 0.01 to 100 parts by weight relative to 1 part by weight of the compound of the present invention.

As the therapeutic agents for diabetes, insulin preparations (e.g., animal insulin preparations extracted from pancreas of bovine and swine; human insulin preparations genetically synthesized using Escherichia coli, yeast; zinc insulin; protamine zinc insulin; fragment or derivative of insulin (e.g., INS-1), oral insulin preparation), insulin sensitizers (e.g., pioglitazone or a salt thereof (preferably hydrochloride), rosiglitazone or a salt thereof (preferably maleate), Netoglitazone, Rivoglitazone (CS-011), FK-614, the compound described in WO01/38325, Tesaglitazar (AZ-242), Ragaglitazar (N,N-622), Muraglitazar (BMS-298585), Edaglitazone (BM-13-1258), Metaglidasen (MBX-102), Naveglitazar (LY-519818), MX-6054, LY-510929, AMG-131(T-131), THR-0921), α-glucosidase inhibitors (e.g., voglibose, acarbose, miglitol, emiglitate etc.), biguanides (e.g., phenformin, metformin, buformin or a salt thereof (e.g., hydrochloride, fumarate, succinate)), insulin secretagogues [sulfonylurea (e.g., tolbutamide, glibenclamide, gliclazide, chlorpropamide, tolazamide, acetohexamide, glyclopyramide, glimepiride, glipizide, glybuzole), repaglinide, nateglinide, mitiglinide or calcium salt hydrate thereof], dipeptidyl peptidase IV inhibitors (e.g., Vidagliptin (LAF237), P32/98, Sitagliptin (MK-431), P93/01, PT-100, Saxagliptin (BMS-477118), T-6666, TS-021), β3 agonists (e.g., AJ-9677), GPR40 agonists, GLP-1 receptor agonists [e.g., GLP-1, GLP-1MR agent, N,N-2211, AC-2993 (exendin-4), BIM-51077, Aib(8,35)hGLP-1 (7,37)NH₂, CJC-[131], amylin agonists (e.g., pramlintide), phosphotyrosine phosphatase inhibitors (e.g., sodium vanadate), gluconeogenesis inhibitors (e.g., glycogen phosphorylase inhibitors, glucose-6-phosphatase inhibitors, glucagon antagonists), SGLUT (sodium-glucose cotransporter) inhibitors (e.g., T-1095), 11β-hydroxysteroid dehydrogenase inhibitors (e.g., BVT-3498), adiponectin or agonists thereof, IKK inhibitors (e.g., AS-2868), leptin resistance improving drugs, somatostatin receptor agonists (compounds described in WO01/25228, WO03/42204, WO98/44921, WO98/45285, WO99/2273.5 etc.), glucokinase activators (e.g., R^{o}-28-1675), GIP (Glucose-dependent insulinotropic peptide) and the like can be mentioned.

Examples of the therapeutic agents for diabetic complications include aldose reductase inhibitors (e.g., Tolrestat, Epalrestat, Zenarestat, Zopolrestat, Minalrestat, Fidarestat, CT-112, ranirestat (AS-3201)), neurotrophic factors and increasing drugs thereof (e.g., NGF, NT-3, BDNF, neurotrophin production-secretion promoters described in WO01/14372 (e.g., 4-(4-chlorophenyl)-2-(2-methyl-1-imidazolyl)-5-[3-(2-methylphenoxy)propyl]oxazole), PKC inhibitors (e.g., ruboxistaurin mesylate)), AGE inhibitors (e.g., ALT946, pimagedine, N-phenacylthiazolium bromide (ALT-766), EX0-226, Pyridorin, Pyridoxamine), active oxygen scavengers (e.g., thioctic acid), cerebral vasodilators (e.g., tiapuride, mexiletine), somatostatin receptor agonist (e.g., BIM23190), apoptosis signal regulating kinase-1 (ASK-1) inhibitors and the like.

Examples of the therapeutic agents for hyperlipidemia include HMG-CoA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, such as N—[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid), fibrate compounds (e.g., bezafibrate, clofibrate, simfibrate, clinofibrate), ACAT inhibitors (e.g., Avasimibe), Eflucimibe)), anion exchange resins (e.g., colestyramine), probucol, nicotinic acid drugs (e.g., nicomol, niceritrol)), ethyl icosapentate, plant sterol (e.g., soysterol), γ-oryzanol) and the like.

Examples of the antihypertensive agents include angiotensin converting enzyme inhibitors (e.g., captopril, enalapril, delapril), angiotensin II antagonists (e.g., candesartan cilexetil, losartan, eprosartan, valsartan, telmisartan, irbesartan, olmesartan medoxomil, tasosartan, 1-[[2′-(2,5-dihydro-5-oxo-4H-1,2,4-oxadiazol-3-yl)biphenyl-4-yl]methyl]-2-ethoxy-1H-benzimidazole-7-carboxylic acid), calcium channel blockers (e.g., manidipine, nifedipine, nicardipine, amlodipine; efonidipine), potassium channel openers (e.g., levcromakalim, L-27152, AL0671, NIP-121), clonidine and the like.

Examples of the antiobesity agents include antiobesity agents acting on the central nervous system (e.g., dexfenfluramine, fenfluramine, phentermine, sibutramine, amfepramone, dexamphetamine, mazindol, phenylpropanolamine, clobenzorex; MCH receptor antagonists (e.g., SB-568849; SNAP-7941; compounds described in WO01/82925 and WO01/87834); neuropeptide γ antagonists (e.g., CP-422935); cannabinoid receptor antagonists (e.g., SR-141716, SR-147778); ghrelin antagonists; 11β-hydroxysteroid dehydrogenase inhibitors (e.g., BVT-3498)), pancreatic lipase inhibitors (e.g., orlistat, cetilistat (ATL-962)), β3 agonists (e.g., AJ-9677), peptide anorexiants (e.g., leptin, CNTF (Ciliary Neurotropic Factor)), cholecystokinin agonists (e.g., lintitript, FPL-15849), feeding deterrents (e.g., P-57) and the like.

Examples of the diuretics include xanthine derivatives (e.g., sodium salicylate and theobromine, calcium salicylate and theobromine), thiazide preparations (e.g., ethiazide, cyclopenthiazide, trichloromethiazide, hydrochlorothiazide, hydroflumethiazide, benzylhydrochlorothiazide, penflutizide, polythiazide, methyclothiazide), antialdosterone preparations (e.g., spironolactone, triamterene), carbonate dehydratase inhibitors (e.g., acetazolamide), chlorobenzenesulfonamide preparations (e.g., chlortalidone, mefruside, indapamide), azosemide, isosorbide, etacrynic acid, piretanide, bumetanide, furosemide and the like.

Examples of the chemotherapeutic agents include alkylating agents (e.g., cyclophosphamide, ifosfamide), metabolic antagonists (e.g., methotrexate, 5-fluorouracil and a derivative thereof), antitumor antibiotics (e.g., mitomycin, adriamycin), plant-derived antitumor agent (e.g., vincristine, vindesine, Taxol), cisplatin, carboplatin, etoposide and the like. Of these, Furtulon or NeoFurtulon, which are 5-fluorouracil derivatives, and the like are preferable.

Examples of the immunotherapeutic agents include microorganism or bacteral components (e.g., muramyl dipeptide derivative, Picibanil), polysaccharides having immunity potentiating activity (e.g., lentinan, schizophyllan, krestin), cytokines obtained by genetic engineering techniques (e.g., interferon, interleukin (IL)), colony stimulating factors (e.g., granulocyte colony stimulating factor, erythropoietin) and the like, with preference given to interleukins such as IL-1, IL-2, IL-12 and the like.

Examples of the antithrombotic agents include heparin (e.g., heparin sodium, heparin calcium, dalteparin sodium), warfarin (e.g., warfarin potassium), anti-thrombin drugs (e.g., aragatroban), thrombolytic agents (e.g., urokinase, tisokinase, alteplase, nateplase, monteplase, pamiteplase), platelet aggregation inhibitors (e.g., ticlopidine hydrochloride, cilostazol, ethyl icosapentate, beraprost sodium, sarpogrelate hydrochloride) and the like.

Examples of the therapeutic agents for osteoporosis include alfacalcidol, calcitriol, elcatonin, calcitonin salmon, estriol, ipriflavone, risedronate disodium, pamidronate disodium, alendronate sodium hydrate, incadronate disodium and the like.

Examples of the antidementia agents include tacrine, donepezil, rivastigmine, galanthamine and the like.

Examples of the erectile dysfunction ameliorating agents include apomorphine, sildenafil citrate and the like.

Examples of the therapeutic agents for urinary incontinence or pollakiuria include flavoxate hydrochloride, oxybutynin hydrochloride, propiverine hydrochloride and the like.

Examples of the therapeutic agents for dysuria include acetylcholine esterase inhibitors (e.g., distigmine) and the like.

Examples of the combination drugs include drugs having a cachexia-ameliorating action established in animal models and clinical situations, such as cyclooxygenase inhibitors (e.g., indomethacin), progesterone derivatives (e.g., megestrol acetate), glucosteroids (e.g., dexamethasone), metoclopramide agents, tetrahydrocannabinol agents, fat metabolism improving agents (e.g., eicosapentanoic acid), growth hormones, IGF-1, or antibodies to a cachexia-inducing factor such as TNF-α, LIF, IL-6, oncostatin M and the like.

As the combination drugs, nerve regeneration promoting drugs (e.g., Y-128, VX853, prosaptide), antidepressants (e.g., desipramine, amitriptyline, imipramine), antiepileptics (e.g., lamotrigine), antiarrhythmic agents (e.g., mexiletine), acetylcholine receptor ligands (e.g., ABT-594), endothelin receptor antagonists (e.g., ABT-627), monoamine uptake inhibitors (e.g., tramadol), narcotic analgesics (e.g., morphine), GABA receptor agonists (e.g., gabapentin), (2 receptor agonists (e.g., clonidine), local analgesics (e.g., capsaicin), antianxiety drugs (e.g., benzothiazepines), dopamine receptor agonists (e.g., apomorphine), midazolam, ketoconazole and the like can also be mentioned.

The combination drug is preferably an insulin preparation, an insulin sensitizer, an α-glucosidase inhibitor, biguanide, insulin secretagogue (preferably sulfonylurea) and the like.

The above-mentioned combination drugs may be used in a mixture of two or more kinds thereof at an appropriate ratio.

When the compound of the present invention is used in combination with a combination drug, the dose of each agent can be reduced within a safe range in consideration of the side effects thereof. Particularly, the doses of insulin sensitizers, insulin secretagogues and biguanides can be reduced from generally dose levels. Therefore, the side effects possibly caused by these agents can be safely prevented. In addition, the doses of the therapeutic agents for diabetic complications, the therapeutic agents for hyperlipidemia and the antihypertensive agents can be reduced, and as a result, the side effects possibly caused by these agents can be effectively prevented.

The production method of the compound of the present invention is explained in the following.

Compound (I) can be produced by a method known per se, for example Method A to Method L, Method R shown below or a method analogous thereto. In the following respective production methods, the starting material compound may be used as a salt, and as such salt, those exemplified as the salts of the compound represented by the formula (I) can be used.

Compound (I-1) of the formula (I) wherein Z is —CONR^{a}SO₂— (R^{a} is as defined above) can be produced, for example, by the following Method A. 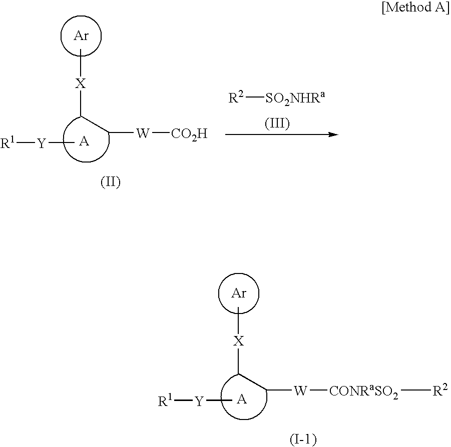
wherein the symbols in the formula are as defined above.

In the present method, compound (I-1) is produced by subjecting compound (II) to an amidation reaction. This reaction is carried out by a method known per se, for example, a method including directly condensing compound (II) with compound (III), or a method including reacting a reactive derivative of compound (II) with compound (III) and the like. Here, as the reactive derivative of compound (II), for example, acid anhydride, acid halide (e.g., acid chloride, acid bromide), imidazolide, mixed acid anhydride (e.g., anhydride with methyl carbonate, ethyl carbonate, isobutyl carbonate, 2,4,6-trichlorobenzoic acid or 2-methyl-6-nitrobenzoic acid) and the like can be mentioned.

The method including directly condensing compound (II) with compound (III) is carried out in the presence of a condensation agent, in a solvent that does not adversely influence the reaction.

As the condensation agent, for example, generally known condensation agents such as carbodiimide condensation reagents (e.g., dicyclohexylcarbodiimide, diisopropylcarbodiimide, 1-ethyl-3-dimethylaminopropylcarbodiimide and hydrochloride thereof); phosphoric acid condensation reagents such as diethyl cyanophosphate, diphenylphosphoryl azide and the like; N,N′-carbonyldiimidazole, 2-chloro-1,3-dimethylimidazolium tetrafluoroborate, chlorodimethoxytriazine and the like can be mentioned.

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; acetonitrile, ethyl acetate, water and the like can be mentioned. These solvents may be used as a mixture thereof at an appropriate ratio.

The amount of compound (III) to be used is generally 0.1 to 10 molar equivalents, preferably 0.3 to 3 molar equivalents, relative to compound (II).

The amount of the condensation agent to be used is generally 0.1 to 10 molar equivalents, preferably 0.3 to 3 molar equivalents, relative to compound (II).

When a carbodiimide condensation reagent is used as the condensation agent, a suitable condensation promoter (e.g., 1-hydroxy-7-azabenzotriazole, 1-hydroxybenzotriazole, N-hydroxysuccinimide, N-hydroxyphthalimide) is used as necessary to improve the reaction efficiency. When a phosphoric acid condensation reagent is used as the condensation agent, an organic amine base such as triethylamine, diisopropylethylamine and the like is generally added to improve the reaction efficiency.

The amount of the above-mentioned condensation promoter and the organic amine base to be used is generally 0.1 to 10 molar equivalents, preferably 0.3 to 3 molar equivalents, relative to compound (II).

The reaction temperature is generally −30° C. to 100° C.

The reaction time is generally 0.5 to 60 hr.

In a method using a reactive derivative of compound (II), when, for example, acid halide is used as the reactive derivative of compound (II), the reaction is carried out in the presence of a base in a solvent that does not adversely influence the reaction.

As the base, for example, amines such as triethylamine, N,N-diisopropylethylamine, N-methylmorpholine, N,N-dimethylaniline, 4-dimethylaminopyridine and the like; alkali metal salts such as sodium hydrogencarbonate, sodium carbonate, potassium carbonate and the like; and the like can be mentioned.

As the solvent that does not adversely influence the reaction, for example, halogenated hydrocarbons such as chloroform, dichloromethane and the like; aromatic hydrocarbons such as benzene, toluene and the like; ethers such as tetrahydrofuran, dioxane, diethyl ether and the like, ethyl acetate, water and the like can be mentioned. These solvents may be used as a mixture thereof at an appropriate ratio.

The amount of compound (III) to be used is generally 0.1 to 10 molar equivalents, preferably 0.3 to 3 molar equivalents, relative to compound (II).

The reaction temperature is generally −30° C. to 100° C.

The reaction time is generally 0.5 to 20 hr.

When mixed acid anhydride is used as the reactive derivative of compound (II), compound (II) is reacted with any of chlorocarbonate (e.g., methyl chlorocarbonate, ethyl chlorocarbonate, isobutyl chlorocarbonate), acid chloride (e.g., 2,4,6-trichlorobenzoyl chloride) and acid anhydride (e.g., 2,4,6-trichlorobenzoic acid anhydride, 2-methyl-6-nitrobenzoic acid anhydride) in the presence of a base (e.g., amines such as triethylamine, N,N-diisopropylethylamine, N-methylmorpholine, N,N-dimethylaniline and the like; alkali metal salts such as sodium hydrogencarbonate, sodium carbonate, potassium carbonate and the like), and then reacted with compound (III).

The amount of compound (III) to be used is generally 0.1 to 10 molar equivalents, preferably 0.3 to 3 molar equivalents, relative to compound (II).

The reaction temperature is generally −30° C. to 100° C.

The reaction time is generally 0.5 to 20 hr.

When imidazolide is used as the reactive derivative of compound (II), compound (II) is reacted with N,N′-carbonyldiimidazole and further reacted with compound (III) in the presence of a base (e.g., amines such as triethylamine, N,N-diisopropylethylamine, N-methylmorpholine, N,N-dimethylaniline, 1,8-diazabicyclo[5.4.0]undeca-7-ene and the like; alkali metal salts such as sodium hydrogencarbonate, sodium carbonate, potassium carbonate and the like).

The amount of compound (III) to be used is generally 0.1 to 110 molar equivalents, preferably 0.3 to 3 molar equivalents, relative to compound (II).

The reaction temperature is generally −30° C. to 100° C.

The reaction time is generally 0.5 to 20 hr.

Compound (III) to be used as a starting material compound in the above-mentioned Method A can be produced according to a method known per se.

Compound (I-2) of the formula (I) wherein Z is —CONR^{a}SO₂NR^{c}— (R^{a} and R^{c} are as defined above) can be produced, for example, by the following Method B. 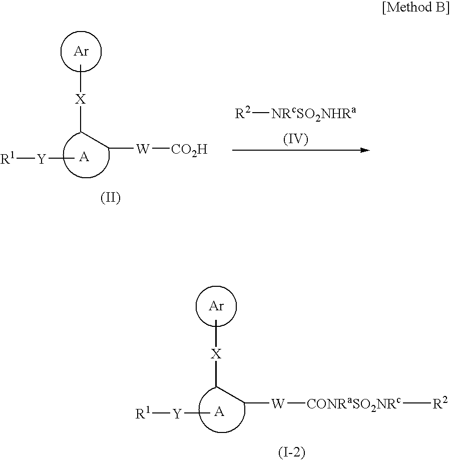
wherein the symbols in the formula are as defined above.

In the present method, compound (II) is reacted with compound (IV) to give compound (I-2). This reaction is carried out in the same manner as in the amidation reaction in the aforementioned Method A.

Compound (IV) can be produced according to a method known per se.

Compound (I-3) of the formula (I) wherein Z is —OCONR^{a}SO₂— (R^{a} is as defined above) can be produced, for example, by the following Method C or Method D. 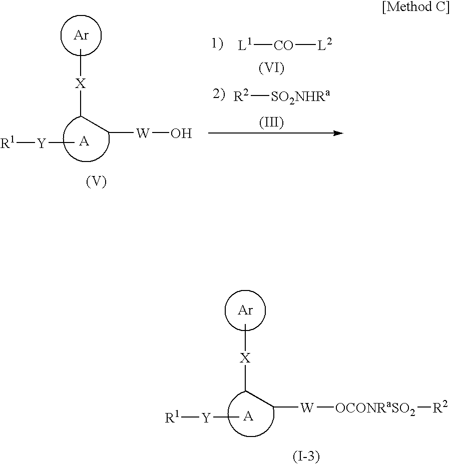
wherein L¹ and L² are the same or different and each is a leaving group, and other symbols are as defined above.

Here, as the leaving group for L¹ or L², for example, a hydroxy group, a halogen atom, imidazolyl group, a succinimidooxy group or —OSO₂R³ (R³ is an alkyl group having 1 to 4 carbon atoms, an aryl group having 6 to 19 carbon atoms optionally substituted by an alkyl group having 1 to 4 carbon atoms) and the like can be mentioned.

As the alkyl group having 1 to 4 carbon atoms of the “alkyl group having 1 to 4 carbon atoms” and the “aryl group having 6 to 10 carbon atoms optionally substituted by an alkyl group having 1 to 4 carbon atoms” for R³, for example, methyl, ethyl, propyl, isopropyl, butyl, isobutyl, sec-butyl and tert-butyl can be mentioned, with preference given to methyl.

As the aryl group having 6 to 10 carbon atoms of the “aryl group having 6 to 10 carbon atoms optionally substituted by an alkyl group having 1 to 4 carbon atoms” for R³, for example, phenyl and naphthyl can be mentioned, with preference given to phenyl.

R³ is particularly preferably methyl, tolyl and the like.

In the present method, compound (I-3) is produced from compound (V). This reaction is carried out by a method known per se, for example, by reacting compound (V) with compound (VI) in a solvent that does not adversely influence the reaction at room temperature for about 0.5 to 5 hr, and reacting the obtained compound with compound (III) in a solvent that does not adversely influence the reaction at room temperature for about 0.5 to 24 hr. Where necessary, this reaction may be carried out in the presence of about 1 to 5 equivalents of a base.

As the base, for example, amines such as triethylamine, N,N-diisopropylethylamine, N-methylmorpholine, N,N-dimethylaniline, pyridine, 4-dimethylaminopyridine and the like; alkali metal salts such as sodium hydrogencarbonate, sodium carbonate, potassium carbonate and the like; and the like can be mentioned.

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; ethyl acetate, water and the like can be mentioned. These solvents may be used as a mixture thereof at an appropriate ratio.

The amount of compound (VI) to be used is generally 0.1 to 10 molar equivalents, preferably 0.3 to 3 molar equivalents, relative to compound (V).

The amount of compound (III) to be used is generally 0.1 to 10 molar equivalents, preferably 0.3 to 3 molar equivalents, relative to compound (V). 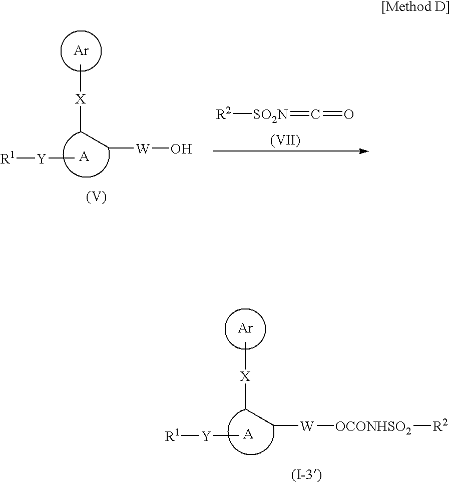
wherein the symbols in the formula are as defined above.

In the present method, compound (V) is reacted with compound (VII) to give, from among compounds (I-3), compound (I-3′) wherein R^{a} is a hydrogen atom. This reaction is carried out by a method known per se, for example, by reacting compound (V) with compound (VII) in a solvent that does not adversely influence the reaction. Where necessary, this reaction may be carried out in the presence of about 1 to 5 equivalents of a base.

As the base, for example, amines such as triethylamine, N,N-diisopropylethylamine, N-methylmorpholine, N,N-dimethylaniline, 4-dimethylaminopyridine and the like; alkali metal salts such as sodium hydrogencarbonate, sodium carbonate, potassium carbonate and the like; and the like can be mentioned.

As the solvent that does not adversely influence the reaction, for example, halogenated hydrocarbons such as chloroform, dichloromethane and the like; aromatic hydrocarbons such as benzene, toluene and the like; ethers such as tetrahydrofuran, dioxane, diethyl ether and the like; acetonitrile, pyridine, ethyl acetate, water and the like can be mentioned. These solvents may be used as a mixture thereof at an appropriate ratio.

The amount of compound (VII) to be used is generally 0.1 to 10 molar equivalents, preferably 0.3 to 3 molar equivalents, relative to compound (V).

The reaction temperature is generally −30° C. to 100° C.

The reaction time is generally 0.5 to 20 hr.

Compound (VII) to be used as a starting material compound in the above-mentioned Method D can be produced according to a method known per se.

Compound (I-4) of the formula (I) wherein Z is —OCONR^{c}— (R^{a} is as defined above) can be produced, for example, by the following Method E. 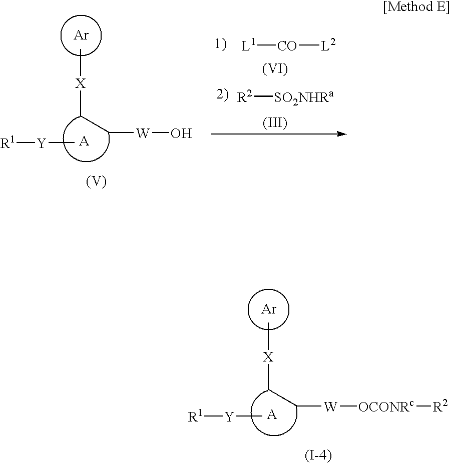
wherein the symbols in the formula are as defined above.

In the present method, compound (I-4) is produced from compound (V). This reaction is carried out by a method known per se, for example, by reacting compound (V) with compound (VI) in a solvent that does not adversely influence the reaction at room temperature for about 0.5 to 5 hr, and reacting the obtained compound with compound (XI) in a solvent that does not adversely influence the reaction at room temperature for about 0.5 to 24 hr. Where necessary, this reaction may be carried out in the presence of about 1 to 5 equivalents of a base.

As the base, for example, amines such as triethylamine, N,N-diisopropylethylamine, N-methylmorpholine, N,N-dimethylaniline, pyridine, 4-dimethylaminopyridine and the like; alkali metal salts such as sodium hydrogencarbonate, sodium carbonate, potassium carbonate and the like; and the like can be mentioned.

As the solvent that does not adversely influence the reaction, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; ethyl acetate, water and the like can be mentioned. These solvents may be used as a mixture thereof at an appropriate ratio.

The amount of compound (VI) to be used is generally 0.1 to 10 molar equivalents, preferably 0.3 to 3 molar equivalents, relative to compound (V).

The amount of compound (XI) to be used is generally 0.1 to 10 molar equivalents, preferably 0.3 to 3 molar equivalents, relative to compound (V).

Compound (XI) to be used as a starting material compound in the above-mentioned Method E can be produced according to a method known per se.

Compound (I-5) of the formula (I) wherein Z is —NR^{a}SO₂— (R^{a} is as defined above) can be produced, for example, by the following Method F or G. 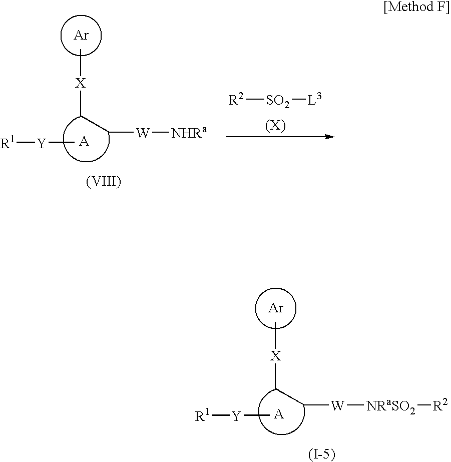
wherein L³ is a leaving group, and other symbols are as defined above.

As the “leaving group” for L³, those exemplified as the aforementioned L¹ and L² can be mentioned. Of those, a halogen atom is preferable and a chlorine atom is particularly preferable.

In the present method, compound (VIII) is reacted with compound (X) to give compound (I-5). This reaction is carried out in the same manner as in the amidation reaction in the aforementioned Method A.

Compound (X) to be used as a starting material compound in the above-mentioned Method F can be produced according to a method known per se. 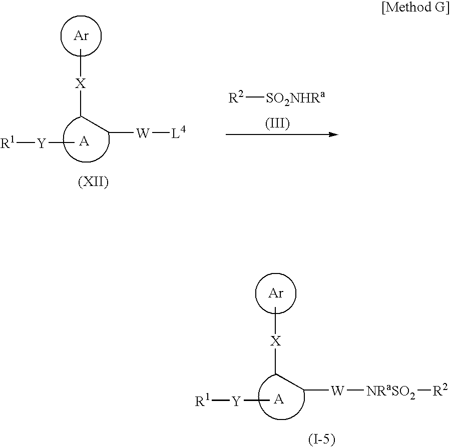
wherein L⁴ is a leaving group, and other symbols are as defined above.

As the “leaving group” for L⁴, those exemplified as the aforementioned L¹ and L² can be mentioned. Of those, a halogen atom and —OSO₂R³ are preferable and a chlorine atom and a methanesulfonyloxy group are particularly preferable.

In the present method, compound (XII) is reacted with compound (III) to give compound (I-5).

When L⁴ is a hydroxy group, this reaction is carried out by a method known per se, for example, the method described in Synthesis, page 1 (1981), or a method analogous thereto. In other words, this reaction is generally carried out in the presence of an organic phosphorus compound and an electrophilic agent in a solvent that does not adversely influence the reaction.

As the organic phosphorus compound, for example, triphenylphosphine, tributylphosphine and the like can be mentioned.

As the electrophilic agent, for example, diethyl azodicarboxylate, diisopropyl azodicarboxylate, 1,1′-azodicarbonyldipiperidine and the like can be mentioned.

The amounts of the organic phosphorus compound and the electrophilic agent to be used are preferably about 1 to about 5 molar equivalents relative to compound (XII).

The amount of compound (III) to be used is preferably about 1 to about 5 molar equivalents relative to compound (XII).

As the solvent that does not adversely influence the reaction, for example, ethers such as diethyl ether, tetrahydrofuran, dioxane and the like; halogenated hydrocarbons such as chloroform, dichloromethane and the like; aromatic hydrocarbons such as benzene, toluene, xylene and the like; amides such as N,N-dimethylformamide and the like; sulfoxides such as dimethyl sulfoxide and the like; and the like can be mentioned. These solvents may be used as a mixture thereof at an appropriate ratio.

The reaction temperature is generally about −50° C. to about 150° C., preferably about −10° C. to about 100° C.

The reaction time is generally about 0.5 to about 20 hr.

When L⁴ is a halogen atom or —OSO₂R³, this reaction is carried out according to a conventional method in the presence of a base in a solvent that does not adversely influence the reaction.

As the base, for example, alkali metal salts such as potassium hydroxide, sodium hydroxide, sodium hydrogencarbonate, potassium carbonate and the like; amines such as pyridine, triethylamine, N,N-diisopropylethylamine, N,N-dimethylaniline, 1,8-diazabicyclo[5.4.0]undec-7-ene and the like; metal hydrides such as potassium hydride, sodium hydride and the like; alkali metal C₁₋₆ alkoxides such as sodium methoxide, sodium ethoxide, potassium tert-butoxide and the like can be mentioned.

The amount of the base to be used is preferably about 1 to about 5 molar equivalents, relative to compound (XII).

The amount of compound (III) to be used is preferably about 1 to about 5 molar equivalents, relative to compound (XII).

As the solvent that does not adversely influence the reaction, for example, aromatic hydrocarbons such as benzene, toluene, xylene and the like; ethers such as tetrahydrofuran, dioxane, diethyl ether and the like; ketones such as acetone, 2-butanone, and the like; halogenated hydrocarbons such as chloroform, dichloromethane and the like; amides such as N,N-dimethylformamide and the like; sulfoxides such as dimethyl sulfoxide and the like and the like can be mentioned. These solvents may be used as a mixture thereof at an appropriate ratio.

The reaction temperature is generally about −50° C. to about 150° C., preferably about −10° C. to about 100° C.

The reaction time is generally about 0.5 to about 20 hr.

Compound (XII) used as the starting material compound in the above-mentioned Method G can be produced by a method known per se from the aforementioned compound (V).

Compound (I-6) of the formula (I) wherein Z is —NR^{a}CONR^{b}SO₂— (R^{a} and R^{b} are as defined above), and R^{b} is a hydrogen atom can be produced, for example, by the following Method H. 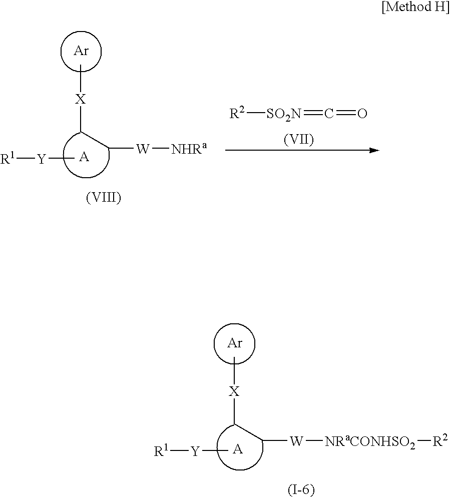
wherein the symbols in the formula are as defined above.

In the present method, compound (VIII) is reacted with compound (VII) to give compound (I-6). This reaction is carried out in the same manner as in the aforementioned Method D.

Compound (I-7) of the formula (I) wherein Z is —OCONR^{a}SO₂NR^{c}— (R^{a} and R^{c}C are as defined above) can be produced, for example, by the following Method I. 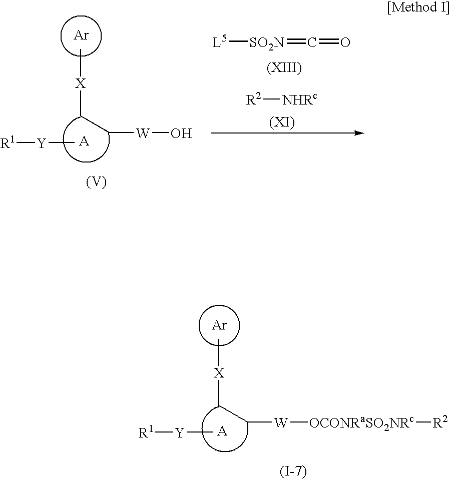
wherein L⁵ is a leaving group, and other symbols are as defined above.

As the “leaving group” for L⁵, those exemplified as the aforementioned L¹ and L² can be mentioned. Of those, a halogen atom is preferable and a chlorine atom is particularly preferable.

In the present method, compound (I-7) is produced from compound (V). This reaction is carried out by a method known per se, for example, by reacting compound (V) with compound (XIII) in a solvent that does not adversely influence the reaction at room temperature for about 0.5 to 5 hr, and reacting the obtained compound with compound (XI) in a solvent that does not adversely influence the reaction at room temperature for about 0.5 to 24 hr. Where necessary, this reaction may be carried out in the presence of about 1 to 5 equivalents of a base.

As the base, for example, amines such as triethylamine, N,N-diisopropylethylamine, N-methylmorpholine, N,N-dimethylaniline, pyridine, 4-dimethylaminopyridine and the like; alkali metal salts such as sodium hydrogencarbonate, sodium carbonate, potassium carbonate and the like, and the like can be mentioned.

As the solvent that does not adversely influence the reaction, for example, halogenated hydrocarbons such as chloroform, dichloromethane and the like; aromatic hydrocarbons such as benzene, toluene and the like; ethers such as tetrahydrofuran, dioxane, diethyl ether and the like, acetonitrile, pyridine, ethyl acetate, water and the like can be mentioned. These solvents may be used as a mixture thereof at an appropriate ratio.

The amount of compound (XIII) to be used is generally 0.1 to 10 molar equivalents, preferably 0.3 to 3 molar equivalents, relative to compound (V)

The amount of compound (XI) to be used is generally 0.1 to 10 molar equivalents, preferably 0.3 to 3 molar equivalents, relative to compound (V).

The reaction temperature is generally −30° C. to 100° C.

The reaction time is generally 0.5 to 20 hr.

Compound (XIII) to be used as a starting material compound in the above-mentioned Method I can be produced according to a method known per se.

Compound (I-8) of the formula (I) wherein Z is —SO₂NR^{a}CO— (R^{a} is as defined above) can be produced, for example, by the following Method J. 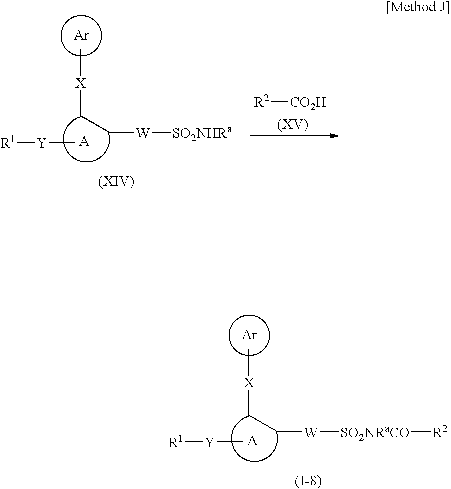
wherein the symbols in the formula are as defined above.

In the present method, compound (I-8) is produced by reacting compound (XIV) with compound (XV). This reaction is carried out in the same manner as in the amidation reaction described in the aforementioned Method A.

Compound (XV) to be used as a starting material compound in the above-mentioned Method J can be produced according to a method known per se. Compound (I-9) of the formula (I) wherein Z is —SO₂NR^{a}COO— (R^{a} is as defined above) can be produced, for example, by the following Method K. 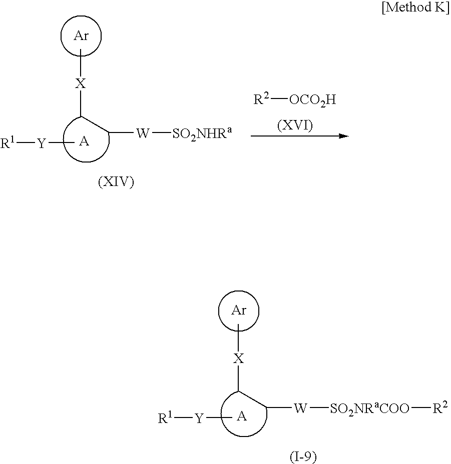
is wherein the symbols in the formula are as defined above.

In the present method, compound (I-9) is produced by reacting compound (XIV) with compound (XVI). This reaction is carried out in the same manner as in the amidation reaction described in the aforementioned Method A.

Compound (XVI) to be used as a starting material compound in the above-mentioned Method K can be produced according to a method known per se.

Compound (I-10) of the formula (I) wherein Z is —NR^{a}SO₂NR^{b}COO— (R^{a} and R^{b} are as defined above) can be produced, for example, by the following Method L. 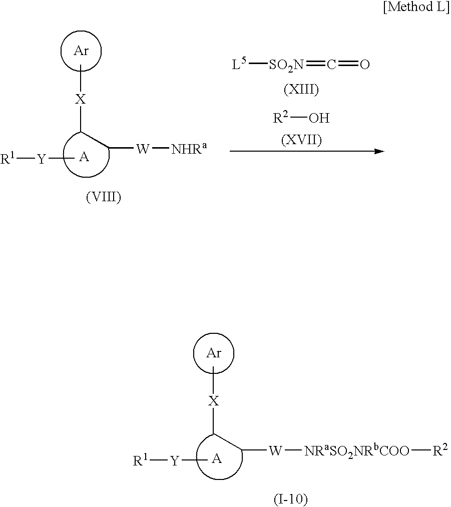
wherein the symbols in the formula are as defined above.

In the present method, compound (I-10) is produced from compound (VIII). This reaction is carried out by a method known per se, for example, by reacting compound (VIII) with compound (XIII) in a solvent that does not adversely influence the reaction at room temperature for about 0.5 to 5 hr, and reacting the obtained compound with compound (XVII) in a solvent that does not adversely influence the reaction at room temperature for about 0.5 to 24 hr. Where necessary, this reaction may be carried out in the presence of about 1 to 5 equivalents of a base.

As the base, for example, amines such as triethylamine, N,N-diisopropylethylamine, N-methylmorpholine, N,N-dimethylaniline, pyridine, 4-dimethylaminopyridine and the like; alkali metal salts such as sodium hydrogencarbonate, sodium carbonate, potassium carbonate and the like, and the like can be mentioned.

As the solvent that does not adversely influence the reaction, for example, halogenated hydrocarbons such as chloroform, dichloromethane and the like; aromatic hydrocarbons such as benzene, toluene and the like; ethers such as tetrahydrofuran, dioxane, diethyl ether and the like, acetonitrile, pyridine, ethyl acetate, water and the like can be mentioned. These solvents may be used as a mixture thereof at an appropriate ratio.

The amount of compound (XIII) to be used is 0.1 to 10 molar equivalents, preferably 0.3 to 3 molar equivalents, relative to compound (VIII).

The amount of compound (XVII) to be used is 0.1 to 10 molar equivalents, preferably 0.3 to 3 molar equivalents, relative to compound (VIII).

The reaction temperature is generally −30° C. to 100° C.

The reaction time is generally 0.5 to 20 hr.

Compound (XVII) to be used as a starting material compound in the above-mentioned Method L can be produced according to a method known per se.

Compound (II) to be used as a starting material compound in the above-mentioned Method A and Method B can be produced, for example, by the following Method M. 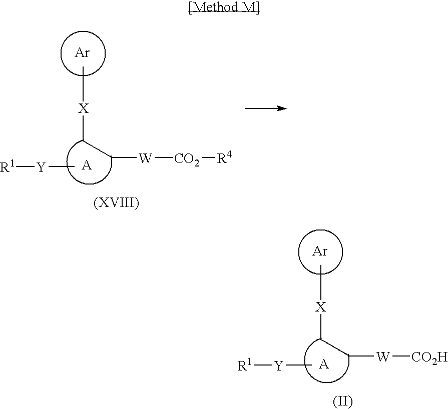
wherein R⁴ is an optionally substituted hydrocarbon group, and other symbols are as defined above.

Here, as the “optionally substituted hydrocarbon group” for the above-mentioned R⁴, those exemplified as the aforementioned R¹ can be mentioned. R⁴ is preferably a C₁₋₆ alkyl group, more preferably methyl, ethyl and the like.

In the present method, compound (II) is produced by subjecting compound (XVIII) to a hydrolysis reaction. This reaction is carried out by a conventional method in the presence of an acid or base in a water-containing solvent.

As the acid, for example, inorganic acids such as hydrochloric acid, sulfuric acid, hydrobromic acid and the like; organic acids such as acetic acid and the like; and the like can be mentioned.

As the base, for example, alkali metal carbonates such as potassium carbonate, sodium carbonate and the like; alkali metal C₁₋₆ alkoxides such as sodium methoxide and the like; alkali metal hydroxides such as potassium hydroxide, sodium hydroxide, lithium hydroxide and the like; and the like can be mentioned.

The amount of the acid or base to be used is generally an excess amount relative to compound (XVIII). The amount of the acid to be used is preferably about 2 to about 50 equivalents relative to compound (XVIII) and the amount of the base to be used is about 1.2 to about 5 equivalents relative to compound (XVIII).

As the water-containing solvent, for example, a mixed solvent of water with one or more kinds of solvents selected from alcohols such as methanol, ethanol and the like; ethers such as tetrahydrofuran, dioxane, diethyl ether and the like; dimethyl sulfoxide, acetone and the like; and the like can be mentioned.

The reaction temperature is generally about −20° C. to about 150° C., preferably about −10° C. to about 100° C.

The reaction time is generally about 0.1 to about 20 hr.

Compound (XVIII) to be used as a starting material compound in the above-mentioned Method M can be produced, for example, by the below-mentioned Method P or a method analogous thereto.

Compound (V) to be used as a starting material compound in the above-mentioned Method C, Method D, Method E and Method I can be produced, for example, by the following Method N. 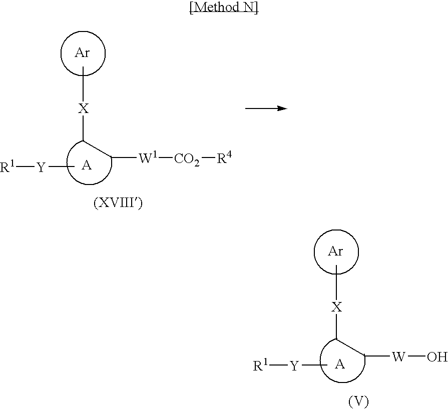
wherein W¹ is optionally substituted divalent hydrocarbon group having 1 to 19 carbon atoms, and other symbols are as defined above.

As the “optionally substituted divalent hydrocarbon group having 1 to 19 carbon atoms” for W¹, the aforementioned “optionally substituted divalent hydrocarbon group having 1 to 20 carbon atoms” for W, which contains 1 to 19 carbon atoms constituting the hydrocarbon group, can be used.

In the present method, compound (V) is produced by subjecting compound (XVIII′) to a reduction reaction. This reaction is generally carried out in the presence of a reducing agent in a solvent that does not adversely influence the reaction.

As the reducing agent, for example, metal hydrogen compounds such as sodium bis(2-methoxyethoxy)aluminum hydride, diisobutylaluminum hydride and the like; metal hydrogen complex compounds such as sodium borohydride, sodium cyanoborohydride, lithium aluminum hydride, sodium aluminum hydride and the like; and the like can be mentioned.

The amount of the reducing agent to be used is generally 1 to 20 molar equivalents relative to compound (XVIII′).

As the solvent that does not adversely influence the reaction, for example, alcohols such as methanol, ethanol, propanol, 2-propanol, butanol, isobutanol, tert-butanol and the like; aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, dimethoxyethane and the like; amides such as N,N-dimethylformamide, N,N-dimethylacetamide, N-methylpyrrolidone and the like; halogenated hydrocarbons such as dichloromethane, chloroform, 1,2-dichloroethane, 1,1,2,2-tetrachloroethane and the like; and the like can be mentioned. These solvents may be used as a mixture thereof at an appropriate ratio.

The reaction temperature is generally −70° C. to 150° C., preferably −20° C. to 100° C.

The reaction time is generally 0.1 to 100 hr, preferably 0.1 to 40 hr.

Compound (XVIII′) to be used as a starting material compound in the above-mentioned Method N can be produced, for example, by the below-mentioned Method P or a method analogous thereto.

Compound (VIII) to be used as a starting material compound in the above-mentioned Method F, Method H and Method L can be produced, for example, by the following Method O. 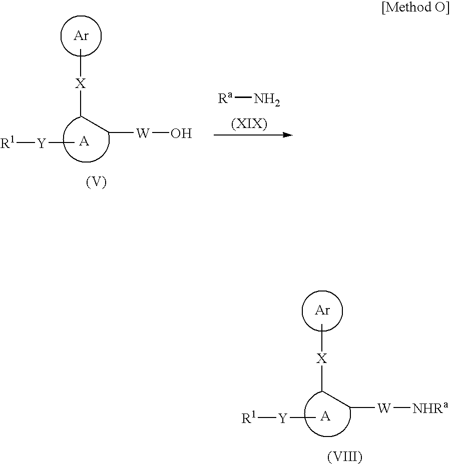
wherein the symbols in the formula are as defined above.

In the present method, compound (VIII) is produced by subjecting compound (V) to an alkylation reaction. This reaction is carried out by a method known per se, by reacting a compound obtained by converting the hydroxy group of compound (V) to a leaving group (halogen atom or —OSO₂R³) with compound (XIX) in the presence of a base in a solvent that does not adversely influence the reaction.

As the base, for example, amines such as triethylamine, N,N-diisopropylethylamine, N-methylmorpholine, N,N-dimethylaniline, pyridine, 4-dimethylaminopyridine and the like; alkali metal salts such as sodium hydrogencarbonate, sodium carbonate, potassium carbonate and the like; metal hydrides such as potassium hydride, sodium hydride and the like; alkali metal C₁₋₆ alkoxides such as sodium methoxide, sodium ethoxide, potassium tert-butoxide and the like; and the like can be mentioned.

As the solvent that does not adversely influence the reaction, for example, halogenated hydrocarbons such as chloroform, dichloromethane and the like; aromatic hydrocarbons such as benzene, toluene and the like; ethers such as tetrahydrofuran, dioxane, diethyl ether and the like, acetonitrile, pyridine, ethyl acetate, water and the like can be mentioned. These solvents may be used as a mixture thereof at an appropriate ratio.

The amount of compound (XIX) to be used is generally 0.1 to 10 molar equivalents, preferably 0.3 to 3 molar equivalents, relative to compound (V).

The reaction temperature is generally −30° C. to 100° C.

The reaction time is generally 0.5 to 20 hr.

Compound (XIX) to be used as a starting material compound in the above-mentioned Method 0 can be produced according to a method known per se.

Of compounds (XVIII) to be used as starting material compounds in the above-mentioned Method M, compound (XVIII-1) and (XVIII-2) wherein W is —CH═CH— or —CH₂CH₂— can be produced, for example, by the following Method P. 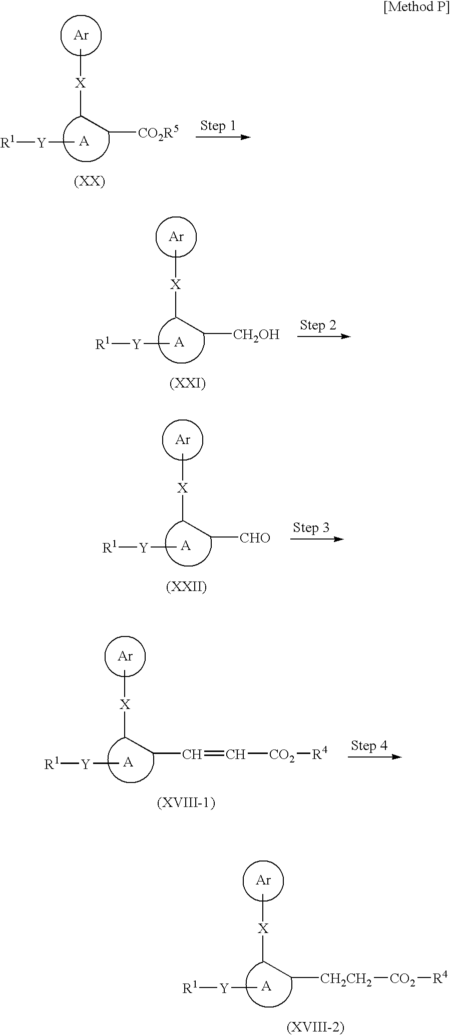
wherein R⁵ is a C₁₋₆ alkyl group, and other symbols are as defined above.
[Step 1]

In this step, compound (XXI) is produced by subjecting compound (XX) to a reduction reaction. This reaction is carried out in the same manner as in the aforementioned Method N.

Compound (XX) can be produced, for example, by the method described in WO 01/38325 and the like, or a method analogous thereto.

[Step 2]

In this step, compound (XXII) is produced by subjecting compound (XXI) to an oxidation reaction. This reaction is generally carried out in the presence of an oxidant in a solvent that does not adversely influence the reaction.

As the oxidant, for example, metal oxidants such as manganese dioxide, pyridinium chlorochlomate, pyridinium dichlorochlomate, ruthenium oxide and the like can be mentioned.

As the solvent that does not adversely influence the reaction, for example, ethers such as diethyl ether, tetrahydrofuran, dioxane and the like; halogenated hydrocarbons such as chloroform, dichloromethane and the like; aromatic hydrocarbons such as benzene, toluene, xylene and the like; and the like can be mentioned. These solvents may be used as a mixture thereof at an appropriate ratio.

The amount of the metal oxidant to be used is generally 1 to 50 molar equivalents, preferably 1 to 10 molar equivalents, relative to compound (XXI).

The reaction temperature is generally about −50° C. to about 150° C., preferably about −10° C. to about 100° C.

The reaction time is generally about 0.5 to about 20 hr.

Compound (XXII) can also be produced by dissolving compound (XXI) in dimethyl sulfoxide or a mixed solvent of dimethyl sulfoxide and halogenated hydrocarbon (e.g., chloroform, dichloromethane) at an appropriate ratio, adding a sulfur trioxide pyridine complex or oxalyl chloride, and reacting with an organic base (e.g., triethylamine, N-methylmorpholine).

The amount of the sulfur trioxide pyridine complex or oxalyl chloride to be used is 1 to 50 molar equivalents, preferably 1 to 10 molar equivalents, relative to compound (XXI).

The amount of the organic base to be used is 1 to 50 molar equivalents, preferably 1 to 10 molar equivalents, relative to compound (XXI).

The reaction temperature is generally about −100° C. to about 150° C., preferably about −70° C. to about 100° C.

The reaction time is generally about 0.5 to about 20 hr.

[Step 3]

In this step, compound (XVIII-1) is produced by subjecting compound (XXII) to a carbon addition reaction. This reaction is generally carried out using an organic phosphorus reagent in a solvent that does not adversely influence the reaction, and in the presence of a base.

As the base, for example, alkali metal salts such as potassium hydroxide, sodium hydroxide, sodium hydrogencarbonate, potassium carbonate and the like; amines such as pyridine, triethylamine, N,N-diisopropylethylamine, N,N-dimethylaniline, 1,8-diazabicyclo[5,4,0]undec-7-ene and the like; metal hydrides such as potassium hydride, sodium hydride and the like; alkali metal C₁₋₆ alkoxides such as sodium methoxide, sodium ethoxide, potassium tert-butoxide and the like can be mentioned.

As the organic phosphorus reagent, for example, trimethyl phosphonoacetate, methyl diethylphosphonoacetate, triethyl phosphonoacetate, tert-butyl diethylphosphonoacetate and the like can be mentioned.

As the solvent that does not adversely influence the reaction, for example, aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, dimethoxyethane and the like; halogenated hydrocarbons such as chloroform, dichloromethane and the like; amides such as N,N-dimethylformamide, N,N-dimethylacetamide, N-methylpyrrolidone and the like; sulfoxides such as dimethyl sulfoxide and the like; alcohols such as methanol, ethanol, isopropanol, tert-butanol and the like; and the like can be mentioned. These solvents may be used as a mixture thereof at an appropriate ratio.

The amount of the base to be used is generally 1 to 50 molar equivalents, preferably 1 to 10 molar equivalents, relative to compound (XXII).

The amount of the organic phosphorus reagent to be used is generally 1 to 50 molar equivalents, preferably 1 to 10 molar equivalents, relative to compound (XXII).

The reaction temperature is generally about −100° C. to about 150° C., preferably about −10° C. to about 100° C.

The reaction time is generally about 0.5 to about 20 hr.

[Step 4]

In this step, compound (XVIII-2) is produced by subjecting compound (XVIII-1) to a hydrogenation reaction.

This reaction can be carried out, for example, in the presence of a metal catalyst such as palladium-carbon, palladium black, palladium chloride, platinum oxide, platinum black, platinum-palladium, Raney-nickel, Raney-cobalt and the like and a hydrogen source, in a solvent that does not adversely influence the reaction.

The amount of the metal catalyst to be used is generally 0.001 to 1000 molar equivalents, preferably 0.01 to 100 molar equivalents, relative to compound (XVIII-1).

As the hydrogen source, for example, hydrogen gas, formic acid, formic acid amine salt, phosphinate, hydrazine and the like can be mentioned.

As the solvent that does not adversely influence the reaction, those exemplified in the aforementioned step 1 can be used.

The reaction temperature and the reaction time are the same as in the aforementioned step 1.

Of the compounds (XIV) to be used as a starting material compound in the above-mentioned Method J and Method K, compound (XIV-1) and (XIV-2) wherein W is —CH═CH— or —CH₂CH₂— and R^{a} is a hydrogen atom can be produced, for example, by the following Method Q. 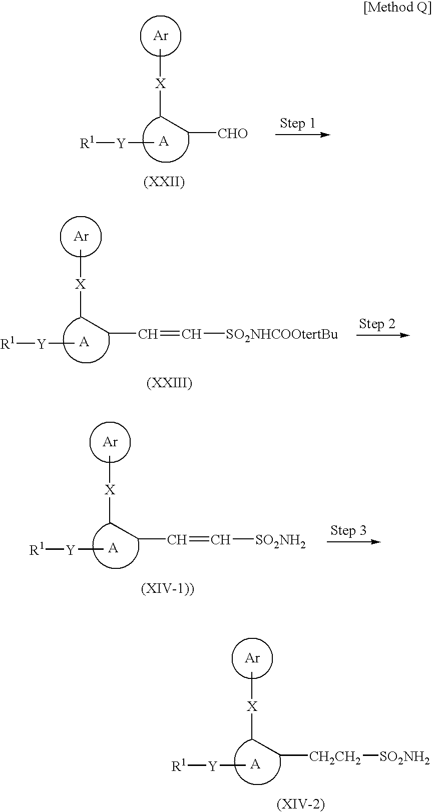
wherein the symbols in the formula are as defined above.
[Step 1]

In this step, compound (XXIII) is produced from compound (XXII). This reaction is carried out by a method known per se, for example, by the method described in Synthesis, page 2321 (2003), or a method analogous thereto.

[Step 2]

In this step, compound (XIV-1) is produced by subjecting compound (XXIII) to a deprotection reaction. This reaction is carried out by a method known per se.

[Step 3]

In this step, compound (XIV-2) is produced by subjecting compound (XIV-1) to a hydrogenation reaction. This reaction is carried out in the same manner as in the reaction described in the aforementioned Method P, step 4.

Compound (I-11) of the formula (I) wherein Y is an oxygen atom or a sulfur atom can be produced, for example, by the following Method R. 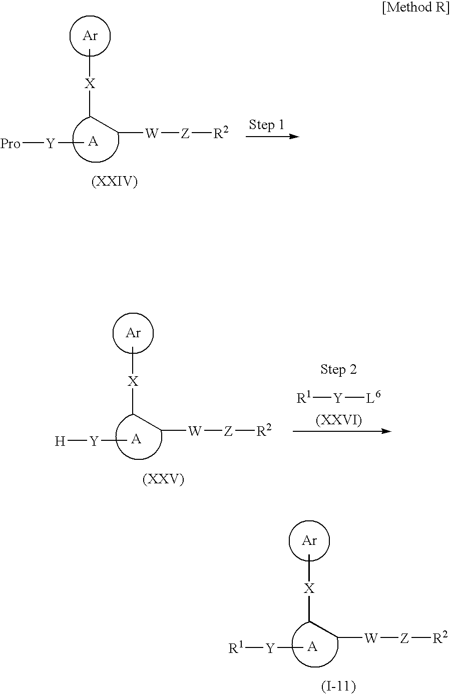
wherein Pro is a hydroxy-protecting group or a mercapto-protecting group, L⁶ is a leaving group, and other symbols are as defined above.

Here, as the hydroxy-protecting group for Pro, for example, a C₁₋₆ alkyl group, a C₇₋₂₀ aralkyl group (e.g., benzyl, trityl), a formyl group, a C₁₋₆ alkyl-carbonyl group, a benzoyl group, a C₇₋₁₀ aralkyl-carbonyl group (e.g., benzylcarbonyl), a 2-tetrahydropyranyl group, a tetrahydrofuranyl group or a substituted silyl group (e.g., a tri-C₁₋₆ alkyl-silyl group such as trimethylsilyl, triethylsilyl, tert-butyldimethylsilyl and tert-butyldiethylsilyl; tert-butyldiphenylsilyl) and the like, each optionally having substituent(s), can be mentioned. As used herein, as the substituent, for example, a halogen atom, a C₁₋₆ alkyl group, a phenyl group, a C₇₋₁₀ aralkyl group (e.g., benzyl), a C₁₋₆ alkoxy group, a nitro group and the like are used. The number of the substituent is 1 to 4.

As the mercapto-protecting group for Pro, for example, a C₁₋₆ alkyl group, a C₇₋₂₀ aralkyl group (e.g., benzyl, trityl) and the like, each optionally having substituent(s), can be mentioned. As used herein, as the substituent, for example, a halogen atom, a C₁₋₆ alkyl group, a phenyl group, a C₇₋₁₀ aralkyl group (e.g., benzyl), a C₁₋₆ alkoxy group, a C₁₋₆ alkyl-carbonyl group, a nitro group and the like are used. The number of the substituent is 1 to 4.

As the “leaving group” for L⁶/those exemplified as the aforementioned L¹ and L² can be mentioned. Of those, a halogen atom or —OSO₂R³ (R³ is an alkyl group having 1 to 4 carbon atoms or an aryl group having 6 to 10 carbon atoms optionally substituted by an alkyl group having 1 to 4 carbon atoms) is preferable, and a chlorine atom and methanesulfonyloxy are particularly preferable.

[Step 1]

In this step, compound (XXV) is produced by subjecting compound (XXIV) to a deprotection reaction. This reaction is carried out by a method known per se.

[Step 2]

In this step, compound (I-11) is produced by reacting compound (XXV) with compound (XXVI). This reaction is carried out in the same manner as in the aforementioned Method G.

Compound (XXIV) to be used as a starting material compound in the above-mentioned Method R can be produced, for example, by any of the aforementioned Method A to Method L.

In addition, compound (XXVI) to be used as a starting material compound in the above-mentioned Method R can be produced according to a method known per se.

In each of the aforementioned reactions, when the starting material compound has an amino group, a carboxyl group, a hydroxy group, a carbonyl group or a mercapto group as a substituent, a protecting group generally used in the peptide chemistry and the like may be introduced into these groups, and the object compound can be obtained by eliminating the protecting group as necessary after the reaction.

The compound of the present invention obtained by each production method mentioned above can be isolated and purified by a known means such as concentration, concentration under reduced pressure, solvent extraction, crystallization, recrystallization, phase transfer, chromatography and the like. Each starting material compound used in each of the above-mentioned production methods can be isolated and purified by a known means similar to those mentioned above. It is also possible to use such starting material compound as it is in a reaction mixture without isolation, as a starting material for the next step.

When compound (I) contains an optical isomer, a stereoisomer, a positional isomer or a rotational isomer, they are also encompassed in compound (I) and can be obtained as single products by synthesis techniques and separation techniques known per se. For example, when compound (I) contains an optical isomer, an optical isomer separated from the compound is also encompassed in compound (I).

The present invention is explained in detail in the following by referring to Experimental Example, Reference Examples, Examples and Formulation Examples, which are not to be construed as limitative.

### EXAMPLES

In the following Reference Examples and Examples, % means wt % unless otherwise specified. In addition, room temperature means a temperature of 1˜30° C. unless otherwise specified.

### Reference Example 1

To a solution of ethyl 3-[2-{[3-chloro-5-(trifluoromethyl)pyridin-2-yl]oxy}-4-(2-methoxyethoxy)phenyl]propionate (5.21 g) in tetrahydrofuran (12 ml) and ethanol (12 ml) was added 1N aqueous sodium hydroxide solution (25 ml), and the mixture was stirred at 50° C. for 30 min. After cooling to room temperature, 1N hydrochloric acid (25 ml) was added to the reaction mixture, and the mixture was extracted with ethyl acetate. The organic layer was washed with saturated brine, dried (MgSO₄), filtrated and concentrated to give a white solid. Recrystallization from ethyl acetate-hexane gave 3-[2-{[3-chloro-5-(trifluoromethyl)pyridin-2-yl]oxy}-4-(2-methoxyethoxy)phenyl]propionic acid (4.15 g, yield: 85%) as white thin needles melting point 116-117° C.

### Reference Example 2

To a solution of ethyl 3-(2-{[3-chloro-5-(trifluoromethyl)pyridin-2-yl]oxy}-4-isopropoxyphenyl)propionate (4.45 g) in tetrahydrofuran (10 ml) and ethanol (10 ml) was added 1N aqueous sodium hydroxide solution (21 ml), and the mixture was stirred at 50° C. for 40 min. After cooling to room temperature, 1N hydrochloric acid (21 ml) was added to the reaction mixture, and the mixture was extracted with ethyl acetate. The organic layer was washed with saturated brine, dried (MgSO₄), filtrated and concentrated to give 3-(2-{[3-chloro-5-(trifluoromethyl)pyridin-2-yl]oxy}-4-isopropoxyphenyl)propionic acid (4.12 g, yield: 99%) as a pale-yellow oil.

¹H-NMR (300 MHz, CDCl₃) δ:1.32 (6H, d, J=6.0 Hz), 2.54-2.69 (2H, m), 2.73-2.83 (2H, m), 4.38-4.59 (1H, m), 6.63 (1H, d, J=2.6 Hz), 6.77 (1H, dd, J=8.6, 2.5 Hz), 7.21 (1H, d, J=8.5 Hz), 7.98 (1H, d, J=2.3 Hz), 8.24-8.28 (1H, m).

### Reference Example 3

To a solution of 2-hydroxy-4-(methoxymethoxy)benzaldehyde (74.68 g) in N,N-dimethylformamide (450 ml) was added sodium hydride (60% in oil, 19.65 g) under ice-cooling, and the mixture was subsequently stirred for 30 min. Then, 2,3-dichloro-5-(trifluoromethyl)pyridine (60.0 ml) was added, and the mixture was stirred at room temperature for 1 hr and at 50° C. for 1 hr. After cooling the reaction mixture to ice temperature, a saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:9-1:2, v/v) to give a pale-yellow solid. Recrystallization from ethyl acetate-hexane gave 2-{[3-chloro-5-(trifluoromethyl)pyridin-2-yl]oxy}-4-(methoxymethoxy)benzaldehyde (79.00 g, yield: 53%) as white crystals. melting point 93.9-94.0° C.

### Reference Example 4

To a solution of ethyl diethylphosphonoacetate (2.76 g) in tetrahydrofuran (10 ml) was added sodium hydride (60% in oil, 482 mg) under ice-cooling, and the mixture was subsequently stirred for 30 min. Then, a solution of 2-{[3-chloro-5-(trifluoromethyl)pyridin-2-yl]oxy}-4-(methoxymethoxy)benzaldehyde (3.90 g) in N,N-dimethylformamide (10 ml) was added dropwise, and the mixture was stirred at room temperature for 30 min. A saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:19-35:65, v/v) to give ethyl (2E)-3-[2-{[3-chloro-5-(trifluoromethyl)pyridin-2-yl]oxy}-4-(methoxymethoxy)phenyl]acrylate (4.66 g, yield: quant.) as a pale-yellow solid. Recrystallization from ethyl acetate-hexane gave white feather crystals. melting point 73.5-74.5° C.

### Reference Example 5

To a solution of ethyl (2E)-3-[2-{[3-chloro-5-(trifluoromethyl)pyridin-2-yl]oxy}-4-(methoxymethoxy)phenyl]acrylate (52.85 g) in acetone (500 ml) was added 1N hydrochloric acid (250 ml), and the mixture was stirred with heating under reflux for 5 hr. After cooling to room temperature, a 1N aqueous sodium hydroxide solution (250 ml) was added to the reaction mixture, and the mixture was diluted with ethyl acetate. The organic layer was washed twice with saturated brine, dried (MgSO₄), filtrated and concentrated. The obtained residue was subjected to silica gel column chromatography, and eluted with ethyl acetate-hexane (1:9-1:4, v/v) to give ethyl (2E)-3-[2-{[3-chloro-5-(trifluoromethyl)pyridin-2-yl]oxy}-4-hydroxyphenyl]acrylate (44.52 g, yield: quant.) as a white solid. Recrystallization from ethyl acetate-hexane gave white crystals. melting point 123.5-124.0° C.

### Reference Example 6

To a solution of ethyl (2E)-3-[2-{[3-chloro-5-(trifluoromethyl)pyridin-2-yl]oxy}-4-hydroxyphenyl]acrylate (41.68 g) in tetrahydrofuran (1000 ml) were added tributylphosphine (49.0 ml), 2-methoxyethanol (13.0 ml) and 1,1′-azodicarbonyldipiperidine (41.01 g), and the mixture was stirred at 50° C. for 20 min. The reaction mixture was concentrated, the obtained solid was washed with diisopropyl ether, and the filtrate was concentrated. The obtained residue was subjected to silica gel column chromatography, and eluted with ethyl acetate-hexane (1:9-1:2, v/v) to give a white solid. Recrystallization from ethyl acetate-hexane gave ethyl (2E)-3-[2-{[3-chloro-5-(trifluoromethyl)pyridin-2-yl]oxy}-4-(2-methoxyethoxy)phenyl]acrylate (28.67 g, yield: 60%) as white feather crystals. melting point 78.2-78.5° C.

### Reference Example 7

To a solution of ethyl (2E)-3-[2-{[3-chloro-5-(trifluoromethyl)pyridin-2-yl]oxy}-4-(2-methoxyethoxy)phenyl]acrylate (2.39 g) in tetrahydrofuran (5.0 ml) and ethanol (5.0 ml) was added a 1N aqueous sodium hydroxide solution (18.0 ml), and the mixture was stirred at 50° C. for 1 hr. After cooling to room temperature, 1N hydrochloric acid (18.0 ml) was added to the reaction mixture, and the mixture was diluted with toluene and concentrated. The residue was dissolved in ethyl acetate, and the organic layer was washed with saturated brine, dried (MgSO₄), filtrated and concentrated to give a white solid. Recrystallization from ethyl acetate-hexane gave. (2E)-3-[2-{[3-chloro-5-(trifluoromethyl)pyridin-2-yl]oxy}-4-(2-methoxyethoxy)phenyl]acrylic acid (1.77 g, yield: 79%) as white feather crystals. melting point 156.5-157.5° C.

### Reference Example 8

To a solution of ethyl (2E)-3-[2-{[3-chloro-5-(trifluoromethyl)pyridin-2-yl]oxy}-4-hydroxyphenyl]acrylate (41.53 g) in N,N-dimethylformamide (10 ml) were added 2-propyl iodide (0.65 ml) and potassium carbonate (712 mg), and the mixture was stirred at 50° C. for 1 hr. After cooling to room temperature, 1N hydrochloric acid was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (hexane alone to 1:3, v/v) to give ethyl (2E)-3-[2-{[3-chloro-5-(trifluoromethyl)pyridin-2-yl]oxy}-4-isopropoxyphenyl]acrylate (1.63 g, yield: 96%) as a white solid. Recrystallization from ethyl acetate-hexane gave white feather crystals. melting point 84.5-85.0° C.

### Reference Example 9

To a solution of ethyl (2E)-3-[2-{[3-chloro-5-(trifluoromethyl)pyridin-2-yl]oxy}-4-isopropoxyphenyl]acrylate (1.53 g) in tetrahydrofuran (10 ml) and ethanol (10 ml) was added a 1N aqueous sodium hydroxide solution (8.0 ml), and the mixture was stirred overnight at room temperature. 1N Hydrochloric acid (8.0 ml) was added to the reaction mixture, and the mixture was diluted with toluene and concentrated. The residue was dissolved in ethyl acetate, and the organic layer was washed with saturated brine, dried (MgSO₄), filtrated and concentrated. The obtained residue was subjected to silica gel column chromatography, and eluted with ethyl acetate-hexane (3:7-3:2, v/v) to give a white solid. Recrystallization from ethyl acetate-hexane gave (2E)-3-[2-{[3-chloro-5-(trifluoromethyl)pyridin-2-yl]oxy}-4-isopropoxyphenyl]acrylic acid (0.91 g, yield: 63%) as a white powder melting point 138.0-139.5° C.

### Reference Example 10

To a solution of 3-[2-{[3-chloro-5-(trifluoromethyl)pyridin-2-yl]oxy}-4-(2-methoxyethoxy)phenyl]propan-1-ol (4.94 g) in ethyl acetate (100 ml) were added triethylamine (3.50 ml) and methanesulfonyl chloride (1.40 ml), and the mixture was stirred at room temperature for 1 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated to give a pale-yellow oil.

To a solution of the obtained oil in N,N-dimethylformamide (50 ml) was added potassium phthalimide (2.46 g), and the mixture was stirred at 80° C. for 3 hr. After cooling to room temperature, water was added to the reaction mixture, and the mixture was extracted with ethyl acetate. The organic layer was washed with water and saturated brine, dried (MgSO₄), filtrated and concentrated to give a pale-yellow solid. The solid was washed with diisopropyl ether to give 2-{3-[2-{[3-chloro-5-(trifluoromethyl)pyridin-2-yl]oxy}-4-(2-methoxyethoxy)phenyl]propyl}-1H-isoindole-1, 3(2H)-dione (6.39 g, yield: 98%). Recrystallization from ethyl acetate-hexane gave white crystals. melting point 101-103° C.

### Reference Example 11

To a solution of 2-{3-[2-{[3-chloro-5-(trifluoromethyl)pyridin-2-yl]oxy}-4-(2-methoxyethoxy)phenyl]propyl}-1H-isoindole-1, 3(2H)-dione (6.12 g) in methanol (100 ml) was added hydrazine monohydrate (4.02 g), and the mixture was stirred at 50° C. for 2 hr. The reaction mixture was concentrated, the residue was washed with diethyl ether, and the filtrate was concentrated. The obtained residue was dissolved in diethyl ether, and the organic layer was washed with saturated aqueous sodium hydrogencarbonate solution and saturated brine, dried (MgSO₄), filtrated and concentrated to give 3-[2-{[3-chloro-5-(trifluoromethyl)pyridin-2-yl]oxy}-4-(2-methoxyethoxy)phenyl]propane-1-amine (2.51 g, yield: 54%) as a dark-brown oil.

¹H-NMR (300 MHz, CDCl₃) δ:1.51-1.95 (4H, m), 2.40-2.55 (2H, m), 2.68 (2H, t, J=7.0 Hz), 3.44 (3H, s), 3.65-3.80 (2H, m), 4.05-4.11 (2H, m), 6.67 (1H, d, J=2.4 Hz), 6.83 (1H, dd, J=8.4, 2.5 Hz), 7.21 (1H, d, J=8.7 Hz), 7.98 (1H, d, J=2.1 Hz), 8.26 (1H, d, J=0.9 Hz).

### Reference Example 12

To a solution of ethyl (2E)-3-[2-{[3-chloro-5-(trifluoromethyl)pyridin-2-yl]oxy}-4-(2-methoxyethoxy)phenyl]acrylate (3.73 g) in tetrahydrofuran (100 ml) was added a 1.5 M solution (19.0 ml) of diisobutylaluminum hydride in toluene under ice-cooling, and the mixture was stirred for 5 min. A saturated aqueous ammonium chloride solution (5.40 ml) was added to the reaction mixture, and the precipitated solid was filtered off and the filtrate was concentrated to give (2E)-3-[2-{[3-chloro-5-(trifluoromethyl)pyridin-2-yl]oxy}-4-(2-methoxyethoxy)phenyl]-2-propen-1-ol (3.26 g, yield: 97%) as a pale-yellow solid. Recrystallization from ethyl acetate-hexane gave white crystals. melting point 119-121° C.

### Reference Example 13

To a solution of 2,4-dihydroxyacetophenone (25.12 g) in tetrahydrofuran (1000 ml) were added triphenylphosphine (70.91 g), 2-methoxyethanol (20.0 ml) and a diethyl azodicarboxylate 40% toluene solution (120 ml) under ice-cooling, and the mixture was stirred at room temperature for 2.5 hr. The reaction mixture was concentrated, and the obtained residue was subjected to silica gel column chromatography, and eluted with ethyl acetate-hexane (1:9-1:4, v/v) to give 1-[2-hydroxy-4-(2-methoxyethoxy)phenyl]ethanone (17.96 g, yield: 52%) as a white solid.

¹H-NMR (300 MHz, CDCl₃) δ:2.56 (3H, s), 3.45 (3H, s), 3.67-3.82 (2H, m), 4.09-4.19 (2H, m), 6.43 (1H, d, J=2.4 Hz), 6.49 (1H, dd, J=8.9, 2.4 Hz), 7.63 (1H, d, J=9.0 Hz), 12.73 (1H, s).

### Reference Example 14

To a solution of 1-[2-hydroxy-4-(2-methoxyethoxy)phenyl]ethanone (5.08 g) in N,N-dimethylformamide (50 ml) were added potassium carbonate (4.71 g) and benzyl bromide (3.50 ml), and the mixture was stirred at room temperature for 3 hr. A saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:19-2:3, v/v) to give 1-[2-(benzyloxy)-4-(2-methoxyethoxy)phenyl]ethanone (7.00 g, yield: 97%) as a colorless oil.

¹H-NMR (300 MHz, CDCl₃) δ:2.55 (3H, s), 3.45 (3H, s), 3.65-3.79 (2H, m), 4.09-4.20 (2H, m), 5.13 (2H, s), 6.45-6.70 (2H, m), 7.32-7.51 (5H, m), 7.84 (1H, d, J=8.7 Hz).

### Reference Example 15

To a solution of ethyl diethylphosphonoacetate (7.34 g) in ethanol (45 ml) was added sodium ethoxide (1.93 g) under ice-cooling, and the mixture was stirred at room temperature for 20 min. To the reaction mixture was added a solution of 1-[2-(benzyloxy)-4-(2-methoxyethoxy)phenyl]ethanone (7.00 g) in ethanol (45 ml), and the mixture was stirred with heating under reflux for 20 hr. After cooling to room temperature, a saturated aqueous ammonium chloride solution was added, and the mixture was diluted with toluene and concentrated. The residue was dissolved in ethyl acetate, and the organic layer was washed with water and saturated brine, dried (MgSO₄), filtrated and concentrated. The obtained residue was subjected to silica gel column chromatography, and eluted with ethyl acetate-hexane (hexane alone to 35:65, v/v) to give ethyl (2E)-3-[2-(benzyloxy)-4-(2-methoxyethoxy)phenyl]-2-butenoate (Reference Example 15a: 6.17 g, yield: 72%) as a colorless oil.

¹H-NMR (300 MHz, CDCl₃) δ:1.29 (3H t, J=7.1 Hz), 2.50 (3H, d, J=1.3 Hz), 3.45 (3H, s), 3.70-3.78 (2H, m), 4.06-4.13 (2H, m), 4.19 (2H, q, J=7.2 Hz), 5.05 (2H, s), 5.93 (1H, d, J=1.3 Hz), 6.48 (1H, dd, J=8.4, 2.4 Hz), 6.59 (1H, d, J=2.3 Hz), 7.11 (1H, d, J=8.3 Hz), 7.28-7.46 (5H, m).

Then, ethyl (2Z)-3-[2-(benzyloxy)-4-(2-methoxyethoxy)phenyl]-2-butenoate (Reference Example 15b: 1.90 g, yield: 22%) was obtained as a colorless oil.

¹H-NMR (300 MHz, CDCl₃) δ:1.06 (3H, t, J=7.1 Hz), 2.15 (3H, d, J=1.3 Hz), 3.44 (3H, s), 3.66-3.79 (2H, m), 3.96 (2H, q, J=7.0. Hz), 4.06-4.14 (2H, m), 5.03 (2H, s), 5.93 (1H, d, J=1.5 Hz), 6.50 (1H, dd, J=8.4, 2.4 Hz), 6.58 (1H, d, J=2.3 Hz), 6.96 (1H, d, J=8.3 Hz), 7.29-7.40 (5H, m).

### Reference Example 16

To a solution of ethyl (2E)-3-[2-(benzyloxy)-4-(2-methoxyethoxy)phenyl]-2-butenoate (6.17 g) in tetrahydrofuran (20 ml) and ethanol (20 ml) was added 10% palladium-carbon (1.91 g), and the mixture was stirred under a hydrogen atmosphere at room temperature for 3 hr. The reaction mixture was filtrated and the filtrate was concentrated to give ethyl 3-[2-hydroxy-4-(2-methoxyethoxy)phenyl]butanoate (4.36 g, yield: 93%) as a colorless oil.

¹H-NMR (300 MHz, CDCl₃) δ:1.19 (3H, t, J=7.2 Hz), 1.30 (3H, d, J=7.2 Hz), 2.49-2.70 (2H, m), 3.44 (3H, s), 3.45-3.56 (1H, m), 3.70-3.74 (2H, m), 4.00-4.16 (4H, m), 6.45-6.49 (1H, m), 6.50-6.59 (1H, m), 6.93 (1H, s), 7.04 (1H, d, J=8.5 Hz)

### Reference Example 17

To a solution of ethyl 3-[2-hydroxy-4-(2-methoxyethoxy)phenyl]butanoate (5.50 g) in N,N-dimethylformamide (40 ml) was added sodium hydride (60% in oil, 932 mg) under ice-cooling, and the mixture was subsequently stirred for 30 min. Then, 2,3-dichloro-5-(trifluoromethyl)pyridine (3.0 ml) was added, and the mixture was stirred at room temperature for 2 hr. A saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (hexane alone to 35:65, v/v) to give ethyl 3-[2-{[3-chloro-5-(trifluoromethyl)pyridin-2-yl]oxy}-4-(2-methoxyethoxy)phenyl]butanoate (7.80 g, yield: 87%) as a yellow oil.

¹H-NMR (300 MHz, CDCl₃) δ:1.16 (3H, t, J=7.2 Hz), 1.22 (3H, d, J=7.0 Hz), 2.39-2.50 (1H, m), 2.56-2.70 (1H, m), 3.23-3.38 (1H, m), 3.43 (3H, s), 3.65-3.79 (2H, m), 3.96-4.19 (4H, m), 6.66 (1H, d, J=2.6 Hz), 6.85 (1H, dd, J=8.7, 2.6 Hz), 7.18-7.29 (1H, m), 7.98 (1H, d, J=2.1 Hz), 8.25 (1H, d, J=1.1 Hz).

### Reference Example 18

To a solution of ethyl 3-[2-{[3-chloro-5-(trifluoromethyl)pyridin-2-yl]oxy}-4-(2-methoxyethoxy)phenyl]butanoate (7.80 g) in tetrahydrofuran (45 ml) was added a 1.0 M solution (80.0 ml) of diisobutylaluminum hydride in hexane under ice-cooling, and the mixture was stirred at room temperature for 1 hr. A saturated aqueous ammonium chloride solution (15.0 ml) was added to the reaction mixture, and the mixture was stirred for 1 hr. The precipitated solid was filtered off and the filtrate was concentrated to give 3-[2-{[3-chloro-5-(trifluoromethyl)pyridin-2-yl]oxy}-4-(2-methoxyethoxy)phenyl]butan-1-ol (7.46 g, yield: quant.) as an orange oil.

¹H-NMR (300 MHz, CDCl₃) δ:1.20 (3H, d, J=7.0 Hz), 1.62 (1H, t, J=6.0 Hz), 1.75-1.87 (2H, m), 2.90-3.05 (1H, m), 3.43 (3H, s), 3.50 (2H, q, J=5.9 Hz), 3.68-3.77 (2H, m), 3.90-4.24 (2H, m), 6.61 (1H, d, J=2.6 Hz), 6.89 (1H, dd, J 8.7, 2.6 Hz), 7.09-7.34 (1H, m), 7.99 (1H, d, J=1.7 Hz), 8.25 (1H, dd, J=2.2, 1.0 Hz).

### Reference Example 19

To a solution of triethyl 2-phosphonopropionate (7.93 g) in tetrahydrofuran (30 ml) was added sodium hydride (60% in oil, 1.31 g) under ice-cooling, and the mixture was subsequently stirred for 30 min. Then, a solution of 2-{[3-chloro-5-(trifluoromethyl)pyridin-2-yl]oxy}-4-(methoxymethoxy)benzaldehyde (10.01 g) in N,N-dimethylformamide (30 ml) was added dropwise, and the mixture was stirred at room temperature for 2 hr. A saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to basic silica gel column chromatography, and eluted with ethyl acetate-hexane (1:4-1:2, v/v) to give ethyl (2E)-3-[2-{[3-chloro-5-(trifluoromethyl)pyridin-2-yl]oxy}-4-(methoxymethoxy)phenyl]-2-methyl acrylate (13.71 g, yield: quant.) as a pale-yellow oil (E/Z=10:1.5).

¹H-NMR (300 MHz, CDCl₃) δ:1.25 (3H, t, J=7.2 Hz), 2.01 (3H, d, J=1.5 Hz), 3.50 (3H, s), 4.17 (2H, q, J=7.2 Hz), 5.20 (2H, s), 6.92 (1H, d, J=2.4 Hz), 7.01 (1H, dd, J=8.7, 2.4 Hz), 7.38 (1H, d, J=8.5 Hz), 7.49 (1H, s), 7.96 (1H, d, J=1.9 Hz), 8.23 (1H, dd, J=2.1, 0.9 Hz).

### Reference Example 20

To a solution of ethyl (2E)-3-[2-{[3-chloro-5-(trifluoromethyl)pyridin-2-yl]oxy}-4-(methoxymethoxy)phenyl]-2-methyl acrylate (13.71 g) in acetone (150 ml) was added 1N hydrochloric acid (60 ml), and the mixture was stirred overnight at 50° C. After cooling to room temperature, a 1N aqueous sodium hydroxide solution (60 ml) was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:9-1:4, v/v) to give ethyl (2E)-3-[2-{[3-chloro-5-(trifluoromethyl)pyridin-2-yl]oxy}-4-hydroxyphenyl]-2-methyl acrylate (10.13 g, yield: 91%) as a white solid. Recrystallization from ethyl acetate-hexane gave white crystals. melting point 101.5-102.0° C.

### Reference Example 21

To a solution of ethyl (2E)-3-[2-{[3-chloro-5-(trifluoromethyl)pyridin-2-yl]oxy}-4-hydroxyphenyl]-2-methyl acrylate (4.96 g) in tetrahydrofuran (100 ml) were added tributylphosphine (6.00 ml), 2-methoxyethanol (1.50 ml) and 1,1′-azodicarbonyldipiperidine (4.51 g), and the mixture was stirred at 50° C. for 30 min. The reaction mixture was concentrated, the obtained solid was washed with diisopropyl ether, and the filtrate was concentrated. The obtained residue was subjected to silica gel column chromatography, and eluted with ethyl acetate-hexane (hexane alone to 2:3, v/v) to give ethyl (2E)-3-[2-{[3-chloro-5-(trifluoromethyl)pyridin-2-yl]oxy}-4-(2-methoxyethoxy)phenyl]-2-methyl acrylate (5.38 g, yield: 95%) as a colorless oil.

¹H-NMR (300 MHz, CDCl₃) δ:1.23-1.29 (3H, m), 2.02 (3H, d, J=1.5 Hz), 3.45 (3H, s), 3.73-3.80 (2H, m), 4.06-4.23 (4H, m), 6.78 (1H, d, J=2.6 Hz), 6.91 (1H, dd, J=8.7, 2.4 Hz), 7.39 (1H, d, J=8.7 Hz), 7.50 (1H, s), 7.96 (1H, d, J=2.1 Hz), 8.22 (1H, d, J=1.1 Hz).

### Reference Example 22

To a solution of ethyl (2E)-3-[2-{[3-chloro-5-(trifluoromethyl)pyridin-2-yl]oxy}-4-(2-methoxyethoxy)phenyl]-2-methyl acrylate (1.76 g) in tetrahydrofuran (40 ml) was added 10% palladium-carbon (1.91 g), and the mixture was stirred under a hydrogen atmosphere at room temperature for 3 hr. The reaction mixture was filtrated. A 1.0 M solution (16.0 ml) of diisobutylaluminum hydride in hexane was added to the filtrate under ice-cooling, and the mixture was stirred at room temperature. After 1 hr, a 1.0 M solution (14.0 ml) of diisobutylaluminum hydride in hexane was added at room temperature, and the mixture was stirred for 1 hr. A saturated aqueous ammonium chloride solution (5.70 ml) was added to the reaction mixture under ice-cooling, and the mixture was stirred for 1 hr. The precipitated solid was filtered off and the filtrate was concentrated to give 3-[2-{[3-chloro-5-(trifluoromethyl)pyridin-2-yl]oxy}-4-(2-methoxyethoxy)phenyl]-2-methylpropan-1-ol (1.41 g, yield: 88%) as a white solid. Recrystallization from ethyl acetate-hexane gave white feather crystals. melting point 84-86° C.

### Reference Example 23

To a solution of ethyl 3-(2-hydroxy-4-isopropoxyphenyl)propionate (10.0 g) in N,N-dimethylformamide (150 ml) was added sodium hydride (60% in oil, 1.92 g) at room temperature, and the mixture was stirred for 30 min. 2,4-Dichlorobenzyl chloride (6.7 ml) was added, and the mixture was stirred at room temperature for 1 hr. Water was added to the reaction mixture, and the mixture was extracted with ethyl acetate. The organic layer was washed with water and saturated brine, dried (MgSO₄), filtrated and concentrated.

The obtained residue was dissolved in tetrahydrofuran (300 ml), lithium aluminum hydride (1.52 g) was added at room temperature, and the mixture was stirred for 30 min. Sodium sulfate 10 hydrate (12.9 g) was added to the reaction mixture, and the resulting solid was filtered off and the filtrate was concentrated. Recrystallization of the residual solid from ethyl acetate-hexane gave 3-{2-[(2,4-dichlorobenzyl)oxy]-4-isopropoxyphenyl}propan-1-ol (11.2 g, yield: 76%) as colorless needles melting point 75.5-76.5° C.

### Reference Example 24

To a solution of 3-{2-[(2,4-dichlorobenzyl)oxy]-4-isopropoxyphenyl}propan-1-ol (2.54 g) in dichloromethane (20 ml) was added Dess-Martin reagent (3.21 g) under ice-cooling, and the mixture was stirred at room temperature for 30 min. A saturated aqueous sodium hydrogencarbonate solution was added to the reaction mixture, and the mixture was diluted with ethyl acetate. The organic layer was washed with saturated brine, dried (MgSO₄), filtrated and concentrated. The obtained residue was subjected to silica gel column chromatography, and eluted with ethyl acetate-hexane (1:2, v/v) to give 3-{2-[(2,4-dichlorobenzyl)oxy]-4-isopropoxyphenyl}propanal (2.88 g, yield: quant.) as a white solid.

¹H-NMR (300 MHz, CDCl₃) δ:1.22-1.39 (6H, m), 2.67-2.83 (2H, m), 2.94 (2H, t, J=7.4 Hz), 4.33-4.60 (1H, m), 5.08 (2H, s), 6.41-6.54 (1H, m), 7.05 (1H, d, J=8.7 Hz), 7.29 (1H, dd, J=8.3, 2.1 Hz), 7.39-7.50 (1H, m), 7.65-7.78 (1H, m), 7.84-8.07 (2H, m), 8.27 (1H, dd, J=7.6, 1.2 Hz), 9.79 (1H, t, J=1.7 Hz).

### Reference Example 25

To a solution of 3-{2-[(2,4-dichlorobenzyl)oxy]-4-isopropoxyphenyl}propanal (2.88 g) in tetrahydrofuran (12 ml), tert-butanol (12 ml) and water (12 ml), sodium dihydrogen phosphate (0.91 g), sodium chlorite (1.90 g) and 2-methyl-2-butene (4.0 ml) were added, and the mixture was stirred overnight at room temperature. 1N Hydrochloric acid was added to the reaction mixture, and the mixture was diluted with ethyl acetate. The organic layer was washed with water and saturated brine, dried (MgSO₄), filtrated and concentrated. Recrystallization of the obtained residue from ethyl acetate-hexane and washing with diisopropyl ether gave 3-{2-[(2,4-dichlorobenzyl)oxy]-4-isopropoxyphenyl}propionic acid (1.03 g, yield: 39%) as pale-yellow crystals.

¹H-NMR (300 MHz, CDCl₃) δ:1.30-1.34 (6H, m), 2.61-2.71 (2H, m), 2.95 (2H, t, J=7.7 Hz), 4.41-4.56 (1H, m), 5.06-5.13 (2H, m), 6.40-6.49 (2H, m), 7.08 (1H, d, J=8.5 Hz), 7.29 (1H, dd, J=8.4, 2.2 Hz), 7.42 (1H, d, J=2.1 Hz), 7.50 (1H, d, J=8.3 Hz).

### Reference Example 26

To a solution of ethyl 3-[2-{[3-chloro-5-(trifluoromethyl)pyridin-2-yl]oxy}-4-(methoxymethoxy)phenyl]propionate (27.52 g) in tetrahydrofuran (320 ml) was added concentrated hydrochloric acid (10.0 ml), and the mixture was stirred at 50° C. for 45 min. After cooling to room temperature, a 1N aqueous sodium hydroxide solution (120 ml) was added to the reaction mixture, and the mixture was diluted with ethyl acetate. The organic layer was washed with water and saturated brine, dried (MgSO₄), filtrated and concentrated. The obtained residue was subjected to silica gel column chromatography, and eluted with ethyl acetate-hexane (1:19-1:4, v/v) to give ethyl 3-[2-{[3-chloro-5-(trifluoromethyl)pyridin-2-yl]oxy}-4-hydroxyphenyl]propionate (20.54 g, yield: 83%) as a pale-yellow oil.

¹H-NMR (300 MHz, CDCl₃) δ:1.09-1.38 (3H, m), 2.44-2.68 (2H, m), 2.68-2.85 (2H, m), 3.97-4.25 (2H, m), 6.61 (1H, d, J=2.2 Hz), 6.65-6.77 (1H, m), 7.18 (1H, d, J=8.1 Hz), 7.98 (1H, d, J=2.2 Hz), 8.26 (1H, s).

### Reference Example 27

To a solution of ethyl 3-[2-{[3-chloro-5-(trifluoromethyl)pyridin-2-yl]oxy}-4-hydroxyphenyl]propionate (0.68 g) in N,N-dimethylformamide (15 ml) were added potassium carbonate (321.5 mg) and 2-chloro-N,N-diethylacetamide (0.31 g), and the mixture was stirred at room temperature for 2 hr, at 50° C. for 30 min, and at 80° C. for 1 hr. 2-Chloro-N,N-diethylacetamide (0.30 g) was added, and the mixture was further stirred for 30 min. After cooling to room temperature, 1N hydrochloric acid was added to the reaction mixture, and the mixture was extracted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:2, v/v) to give ethyl 3-{2-{[3-chloro-5-(trifluoromethyl)pyridin-2-yl]oxy}-4-[2-(diethylamino)-2-oxoethoxy]phenyl}propionate (1.39 g, yield: quant.) as a white solid. Recrystallization from ethyl acetate-hexane gave white cotton crystals. melting point 88.5-89.0° C.

### Reference Example 28

To a solution of 1-bromo-3-methoxypropane (19.50 g) in N,N-dimethylformamide (200 ml) was added potassium thioacetate (15.15 g), and the mixture was stirred overnight at room temperature. Water was added to the reaction mixture, and the mixture was extracted with diethyl ether. The organic layer was washed with water and saturated brine, dried (MgSO₄), filtrated and concentrated to give a yellow oil.

To a solution of the obtained oil in diethyl ether (200 ml) was added 1N aqueous sodium hydroxide solution (250 ml) under ice-cooling, and the mixture was stirred under ice-cooling. After 1 hr, a 12N aqueous sodium hydroxide solution (20 ml) and methanol (50 ml) were added, and the mixture was stirred at room temperature for 20 min. Concentrated hydrochloric acid (45 ml) was added to the reaction mixture, and the mixture was extracted with diethyl ether. The organic layer was washed with water, a saturated aqueous sodium hydrogencarbonate solution and saturated brine, dried (MgSO₄), filtrated and concentrated.

Into a solution of the obtained residue in acetic acid (200 ml) and water (200 ml) was blown gaseous chlorine over 2 hr so that the temperature in the system would not rise to 15° C. or above. Gaseous nitrogen was blown in at room temperature for 1 hr, the reaction mixture was added dropwise to an ice-cooled saturated aqueous sodium hydrogencarbonate solution, and the mixture was extracted with diethyl ether. The organic layer was washed with a saturated aqueous sodium hydrogencarbonate solution, a 10% aqueous sodium thiosulfate solution and saturated brine, dried (MgSO₄), filtrated and concentrated to give a pale-yellow oil.

A solution of the obtained oil in tetrahydrofuran (10 ml) was added dropwise to a solution of 28% aqueous ammonia (50 ml) and tetrahydrofuran (50 ml) under ice-cooling, and the mixture was subsequently stirred for 30 min. The reaction mixture was concentrated and the concentrate was dissolved in ethyl acetate. The organic layer was washed with water and saturated brine, dried (MgSO₄), filtrated and concentrated to give an orange oil. The obtained oil was dissolved in a suspension of activated carbon in ethyl acetate, and the mixture was stirred at room temperature for 1 hr. The mixture was filtrated and concentrated to give 3-methoxypropane-1-sulfonamide (0.88 g, yield: 5%) as an orange solid.

¹H-NMR (300 MHz, CDCl₃) δ:1.86-2.25 (2H, m), 3.19-3.28 (2H, m), 3.35 (3H, s), 3.53 (2H, t, J=5.8 Hz), 4.71 (2H, s).

### Reference Example 29

To a solution of ethyl (2E)-3-[2-(benzyloxy)-4-hydroxyphenyl]acrylate (2.01 g) in tetrahydrofuran (100 ml) were added triphenylphosphine (2.65 g), 1-(2-hydroxyethyl)-2-pyrrolidone (0.80 ml) and a diethyl azodicarboxylate 40% toluene solution (5.50 ml) under ice-cooling, and the mixture was stirred at room temperature for 3 hr. The reaction mixture was concentrated, and the obtained residue was subjected to silica gel column chromatography and eluted with ethyl acetate-hexane (1:3 to ethyl acetate alone, v/v), then ethyl acetate-methanol (ethyl acetate alone to 9:1, v/v) to give ethyl (2E)-3-{2-(benzyloxy)-4-[2-(2-oxopyrrolidin-1-yl)ethoxy]phenyl}acrylate (4.01 g, yield: quant.) as a white solid. Recrystallization from ethyl acetate-hexane gave white crystals. melting point 94-97° C.

### Reference Example 30

To a solution of (2E)-3-{2-(benzyloxy)-4-[2-(2-oxopyrrolidin-1-yl)ethoxy]phenyl}ethyl acrylate (3.81 g) in tetrahydrofuran (25 ml) and ethanol (25 ml) was added 10% palladium-carbon (1.00 g), and the mixture was stirred overnight under a hydrogen atmosphere at room temperature. The reaction mixture was filtrated and the filtrate was concentrated to give ethyl 3-{2-hydroxy-4-[2-(2-oxopyrrolidin1-yl)ethoxy]phenyl}propionate (3.05 g, yield: quant.) as a pale-brown solid. Recrystallization from ethyl acetate-diisopropyl ether gave a white powder melting point 112-115° C.

### Reference Example 31

To a solution of ethyl 3-{2-hydroxy-4-[2-(2-oxopyrrolidin-1-yl)ethoxy]phenyl}propionate (2.90 g) in N,N-dimethylformamide (50 ml) was added sodium hydride (60% in oil, 464 mg) under ice-cooling, and the mixture was subsequently stirred for 30 min. Then, 2,3-dichloro-5-(trifluoromethyl)pyridine (1.50 ml) was added, and the mixture was stirred at room temperature for 1 hr. A saturated aqueous ammonium chloride solution was added to the reaction mixture, and the mixture was extracted with ethyl acetate. The organic layer was washed with saturated brine, dried (MgSO₄), filtrated and concentrated. The obtained residue was subjected to silica gel column chromatography, and eluted with ethyl acetate-hexane (1:1 to ethyl acetate alone, v/v) to give an orange oil.

To a solution of the obtained oil in tetrahydrofuran (15 ml) and ethanol (15 ml) was added a 1N aqueous sodium hydroxide solution (16.0 ml), and the mixture was stirred overnight at room temperature. 1N Aqueous sodium hydroxide solution (15.0 ml) was added and, after 1 hr, a 1N aqueous sodium hydroxide solution (10.0 ml) was further added, and the mixture was stirred for 1 hr. 1N Hydrochloric acid (41.0 ml) was added to the reaction mixture, and the mixture was concentrated. The residue was dissolved in ethyl acetate. The organic layer was washed with saturated brine, dried (MgSO₄), filtrated and concentrated to give a white solid. The obtained residue was subjected to silica gel column chromatography, and eluted with ethyl acetate-hexane (3:7 to ethyl acetate alone, v/v), then ethyl acetate-methanol (ethyl acetate alone to 9:1, v/v), to give 3-{2-{[3-chloro-5-(trifluoromethyl)pyridin-2-yl]oxy}-4-[2-(2-oxopyrrolidin-1-yl)ethoxy]phenyl}propionic acid (0.85 g, 27%) as a white solid. Recrystallization from ethyl acetate-hexane gave a white powder melting point 145.0-145.5° C.

### Reference Example 32

To a solution of ethyl 3-[2-{[3-chloro-5-(trifluoromethyl)pyridin-2-yl]oxy}-4-hydroxyphenyl]propionate (3.26 g) in dichloromethane (40 ml) were added 2,6-lutidine (2.0 ml) and triisopropylsilyl trifluoromethanesulfonate (2.50 ml) under ice-cooling, and the mixture was stirred for 30 min. Under ice-cooling, 2,6-lutidine (2.0 ml) and triisopropylsilyl trifluoromethanesulfonate (2.50 ml) were further added, and the mixture was stirred for 15 min. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-35:65, v/v) to give a yellow oil.

To a solution of the obtained oil in tetrahydrofuran (40 ml) was added a 1.5 M solution (20.0 ml) of diisobutylaluminum hydride in toluene under ice-cooling, and the mixture was stirred for 15 min. A saturated aqueous ammonium chloride solution (5.80 ml) was added to the reaction mixture, and the precipitated solid was filtered off and the filtrate was concentrated. The obtained residue was subjected to silica gel column chromatography, and eluted with ethyl acetate-hexane (1:19-3:7, v/v) to give a yellow oil.

To a solution of the obtained oil in N,N-dimethylformamide (10 ml) was added N,N′-carbonyldiimidazole (345 mg), and the mixture was stirred at 40° C. for 1 hr. Cyclopropylmethylamine (834 mg) was added to the reaction mixture, and the mixture was stirred at 50° C. for 5 hr. After cooling to room temperature, 1N hydrochloric acid was added to the reaction mixture, and the mixture was extracted with ethyl acetate. The organic layer was washed with 1N hydrochloric acid, a saturated aqueous sodium hydrogencarbonate solution and saturated brine, dried (MgSO₄), filtrated and concentrated to give an orange oil.

To a solution of the obtained oil in tetrahydrofuran (4 ml) was added tetrabutylammoniumfluoride 1.0 M tetrahydrofuran solution (4.0 ml), and the mixture was stirred at room temperature for 1 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give 3-(2-{[3-chloro-5-(trifluoromethyl)pyridin-2-yl]oxy}-4-hydroxyphenyl)propyl (cyclopropylmethyl)carbamate (749 mg, yield: 39%) as a pale-orange oil.

¹H-NMR (300 MHz, CDCl₃) δ:0.11-0.25 (2H, m), 0.41-0.56° (2H, m), 0.72-1.19 (1H, m), 1.78-1.95 (2H, m), 2.50 (2H, t, J=7.5 Hz), 2.95-3.07 (2H, m), 4.02 (2H, t, J=6.1 Hz), 4.71 (1H, s), 6.58-6.63 (1H, m), 6.67-6.79 (1H, m), 7.12-7.19 (1H, m), 7.98 (1H, d, J=2.3 Hz), 8.27 (1H, dd, J=2.0, 1.0 Hz).

### Reference Example 33

To a solution of 2,4-dihydroxybenzaldehyde (25.29 g) in tetrahydrofuran (1000 ml) were added triphenylphosphine (70.34 g), 2-methoxyethanol (20.0 ml) and a diethyl azodicarboxylate 40% toluene solution (120 ml) under ice-cooling, and the mixture was stirred overnight at room temperature. The reaction mixture was concentrated, and the obtained residue was subjected to silica gel column chromatography, and eluted with ethyl acetate-hexane (1:9-1:4, v/v) to give a white solid. Recrystallization from ethyl acetate-hexane gave 2-hydroxy-4-(2-methoxyethoxy)benzaldehyde (12.65 g, yield: 35%) as white fine needles melting point 64.5-65.5° C.

### Reference Example 34

To a solution of 2-hydroxy-4-(2-methoxyethoxy)benzaldehyde (12.86 g) in N,N-dimethylformamide (100 ml) was added sodium hydride (60% in oil, 3.24 g) under ice-cooling, and the mixture was subsequently stirred for 30 min. Then, 2,3-dichloro-5-(trifluoromethyl)pyridine (12.0 ml) was added, and the mixture was stirred at room temperature for 3 hr and at 50° C. for 30 min. After cooling the reaction mixture to room temperature, a saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:9-1:4, v/v). Activated carbon was suspended in the eluted fraction, and the suspension was stirred for 1 hr, filtrated and concentrated to give 2-{[3-chloro-5-(trifluoromethyl)pyridin-2-yl]oxy}-4-(2-methoxyethoxy)benzaldehyde (14.66 g, yield: 60%) as a pale-yellow powder. Recrystallization from ethyl acetate-hexane gave a white prism melting point 80.5-81.0° C.

### Reference Example 35

To a solution of 2-{[3-chloro-5-(trifluoromethyl)pyridin-2-yl]oxy}-4-(2-methoxyethoxy)benzaldehyde (13.04 g) in tetrahydrofuran (50 ml) and methanol (30 ml) was added sodium borohydride (3.99 g) under ice-cooling, and the mixture was stirred at room temperature for 1 hr. Sodium borohydride (1.21 g) was further added, and the mixture was stirred for 1 hr. 1N Hydrochloric acid was added to the reaction mixture, and the mixture was diluted with ethyl acetate. The organic layer was washed with water and saturated brine, dried (MgSO₄), filtrated and concentrated. The obtained residue was subjected to silica gel column chromatography, eluted with ethyl acetate-hexane (1:4-1:1, v/v) and then to basic silica gel column chromatography, and eluted with ethyl acetate-hexane (1:1 to ethyl acetate alone, v/v) to give [2-{[3-chloro-5-(trifluoromethyl)pyridin-2-yl]oxy}-4-(2-methoxyethoxy)phenyl]methanol (2.51 g, yield: 19%) as a white solid. Recrystallization from ethyl acetate-hexane gave white feather crystals. melting point 85.0-85.5° C.

### Reference Example 36

To a solution of 2-{[3-chloro-5-(trifluoromethyl)pyridin-2-yl]oxy}-4-(2-methoxymethoxy)benzaldehyde (26.42 g) in tetrahydrofuran (500 ml) was added concentrated hydrochloric acid (50.0 ml), and the mixture was stirred at room temperature for 17 hr. A 1N aqueous sodium hydroxide solution (600 ml) was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:4-1:2, v/v) to give 2-{[3-chloro-5-(trifluoromethyl)pyridin-2-yl]oxy}-4-hydroxybenzaldehyde (22.63 g, yield: 98%) as a pale-pink solid. Recrystallization from ethyl acetate-hexane gave white crystals. melting point 129° C. (dec.)

### Reference Example 37

To a solution of 2-{[3-chloro-5-(trifluoromethyl)pyridin-2-yl]oxy}-4-hydroxybenzaldehyde (3.12 g) in N,N-dimethylformamide (20 ml) was added potassium carbonate (2.25 g) and 1-iodobutane (1.50 ml), and the mixture was stirred at 50° C. for 1 hr. After cooling the reaction mixture to room temperature, the mixture was extracted with a saturated aqueous ammonium chloride solution and ethyl acetate. The organic layer was washed with water and saturated brine, dried (MgSO₄), filtrated and concentrated. The obtained residue was subjected to silica gel column chromatography, and eluted with ethyl acetate-hexane (1:19-2:3, v/v) to give a pale-yellow oil.

To a solution of the obtained oil in tetrahydrofuran (15 ml) and methanol (15 ml) was added sodium borohydride (895 mg) under ice-cooling, and the mixture was stirred for 30 min. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The concentrate was subjected to basic silica gel column chromatography, and eluted with ethyl acetate-hexane (1:9-1:1, v/v) to give (4-butoxy-2-{[3-chloro-5-(trifluoromethyl)pyridin-2-yl]oxy}phenyl)methanol (2.76 g, yield: 75%) as a white solid. Recrystallization from ethyl acetate-hexane gave white fine needles melting point 77.8-79.2° C.

### Reference Example 38

To a solution of 2-{[3-chloro-5-(trifluoromethyl)pyridin-2-yl]oxy}-4-hydroxybenzaldehyde (3.09 g) in N,N-dimethylformamide (20 ml) was added potassium carbonate (2.08 g) and 2-iodopropane (1.50 ml), and the mixture was stirred at 50° C. for 1.5 hr. After cooling the reaction mixture to room temperature, the mixture was extracted with a saturated aqueous ammonium chloride solution and ethyl acetate. The organic layer was washed with water and saturated brine, dried (MgSO₄), filtrated and concentrated. The obtained residue was subjected to silica gel column chromatography, and eluted with ethyl acetate-hexane (1:19-45:55, v/v) to give a pale-yellow oil.

To a solution of the obtained oil in tetrahydrofuran (15 ml) and methanol (15 ml) was added sodium borohydride (912 mg) under ice-cooling, and the mixture was stirred for 30 min. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The concentrate was subjected to basic silica gel column chromatography, and eluted with ethyl acetate-hexane (1:9-1:1, v/v) to give (2-{[3-chloro-5-(trifluoromethyl)pyridin-2-yl]oxy}-4-isopropoxyphenyl)methanol (2.48 g, yield: 71%) as a white solid. Recrystallization from ethyl acetate-hexane gave a white powder melting point 63.0-64.0° C.

### Reference Example 39

To a solution of 2-{[3-chloro-5-(trifluoromethyl)pyridin-2-yl]oxy}-4-hydroxybenzaldehyde (3.09 g) in tetrahydrofuran (50 ml) were added pyridine (5.0 ml) and 1-propanesulfonyl chloride (1.60 ml), and the mixture was stirred at room temperature for 30 min. Then, triethylamine (5.0 ml), 1-propanesulfonyl chloride (1.60 ml) and ethyl acetate (50 ml) were added, and the mixture was stirred at room temperature for 2 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give a yellow oil.

To a solution of the obtained oil in methanol (30 ml) was added sodium borohydride (385.1 mg) under ice-cooling, and the mixture was stirred for 20 min. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The concentrate was subjected to basic silica gel column chromatography, and eluted with ethyl acetate-hexane (1:9-3:2, v/v) to give 3-{[3-chloro-5-(trifluoromethyl)pyridin-2-yl]oxy}-4-(hydroxymethyl)phenyl propane-1-sulfonate (2.44 g, yield: 59%) as a white solid. Recrystallization from ethyl acetate-hexane gave white feather crystals. melting point 68.5-69.5° C.

### Reference Example 40

A solution of sulfamide (811 mg) and N-methylhexylamine (1.00 ml) in 1,2-dimethoxyethane (25 ml) was stirred overnight with heating under reflux. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated to give N-hexyl-N-methylsulfamide (0.67 g, yield: 52%) as a pale-yellow solid.

¹H-NMR (300 MHz, CDCl₃) δ:0.80-0.96 (3H, m), 1.10-1.43 (6H, m), 1.46-1.62 (2H, m), 2.80 (3H, s), 2.98-3.17 (2H, m), 4.56 (2H, s).

### Reference Example 41

To a solution of 2-hydroxy-4-(2-methoxyethoxy)benzaldehyde (9.30 g) in N,N-dimethylformamide (100 ml) were added potassium carbonate (6.91 g) and benzyl bromide (7.0 ml), and the mixture was stirred overnight at room temperature. Water was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:9-1:4, v/v) to give a white solid. Recrystallization from ethyl acetate-hexane gave 2-(benzyloxy)-4-(2-methoxyethoxy)benzaldehyde (10.71 g, yield: 79%) as white crystals. melting point 67.5-68.0° C.

### Reference Example 42

To a solution of [2-(1,3-dioxolan-2-yl)ethyl]triphenylphosphoniumbromide (6.96 g) in N,N-dimethylformamide (50 ml) was added sodium hydride (60% in oil, 628 mg) under ice-cooling, and the mixture was subsequently stirred for 30 min. Then, a solution of 2-(benzyloxy)-4-(2-methoxyethoxy)benzaldehyde (3.12 g) in N,N-dimethylformamide (30 ml) was added dropwise, and the mixture was stirred at room temperature for 1 hr and at 80° C. for 5 hr. A saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:19-2:3, v/v) to give a colorless oil.

To a solution of the obtained oil in ethanol (50 ml) was added 10% palladium-carbon (3.16 g), and the mixture was stirred under a hydrogen atmosphere at room temperature for 3 hr. The reaction mixture was filtrated, and the filtrate was concentrated. The obtained residue was subjected to silica gel column chromatography, and eluted with ethyl acetate-hexane (1:9-1:4, v/v) to give 2-[3-(1,3-dioxolan-2-yl)propyl]-5-(2-methoxyethoxy)phenol (0.93 g, yield: 30%) as a pale-yellow oil.

H-NMR (300 MHz, CDCl₃) δ:1.64-1.87 (4H, m), 2.61 (2H, t, J=6.8 Hz), 3.44 (3H, s), 3.64-3.77 (2H, m), 3.84-3.94 (2 H, m), 3.94-4.26 (4H, m), 4.91 (1H, t, J=4.3 Hz), 6.22 (1H, s), 6.33-6.62 (2H, m), 6.96 (1H, d, J=8.1 Hz).

### Reference Example 43

To a solution of 2-[3-(1,3-dioxolan-2-yl)propyl]-5-(2-methoxyethoxy)phenol (0.93 g) in N,N-dimethylformamide (10 ml) was added sodium hydride (60% in oil, 155 mg) under ice-cooling, and the mixture was subsequently stirred for 30 min. Then, 2,3-dichloro-5-(trifluoromethyl)pyridine (0.50 ml) was added, and the mixture was stirred at room temperature for 30 min. A saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:19-1:1, v/v) to give 3-chloro-2-[2-[3-(1,3-dioxolan-2-yl)propyl]-5-(2-methoxyethoxy)phenoxy]-5-(trifluoromethyl)pyridine (1.33 g, yield: 87%) as a white solid. Recrystallization from ethyl acetate-hexane gave a white powder melting point 92.0-92.5° C.

### Reference Example 44

A solution of 3-chloro-2-[2-[3-(1,3-dioxolan-2-yl)propyl]-5-(2-methoxyethoxy)phenoxy]-5-(trifluoromethyl)pyridine (1.08 g) in a 80% aqueous acetic acid solution (15 ml) was stirred at 50° C. for 4 hr and at 80° C. for 3 hr. The reaction mixture was concentrated, and the obtained residue was subjected to silica gel column chromatography, and eluted with ethyl acetate-hexane (1:19-2:3, v/v) to give a pale-yellow oil.

To a solution of the obtained oil in tert-butanol (10 ml) and water (2.5 ml) were added sodium dihydrogen phosphate (313 mg), sodium chlorite (645 mg) and 2-methyl-2-butene (4.5 ml) at room temperature, and the mixture was stirred at room temperature for 30 min. A 10% aqueous sodium hydrogen sulfite solution and 1N hydrochloric acid were added to the reaction mixture, and the mixture was extracted with ethyl acetate. The organic layer was washed with water and saturated brine, dried (MgSO₄), filtrated and concentrated. Recrystallization of the obtained residue from ethyl acetate-hexane gave 4-[2-{[3-chloro-5-(trifluoromethyl)pyridin-2-yl]oxy}-4-(2-methoxyethoxy)phenyl]butanoic acid (612 mg, yield: 60%) as white fine needles melting point 119.0-119.5° C.

### Reference Example 45

To a solution of 3-[2-{[3-chloro-5-(trifluoromethyl)pyridin-2-yl]oxy}-4-(2-methoxyethoxy)phenyl]butan-1-ol (2.63 g) in dichloromethane (30 ml) was added a Dess-Martin reagent (3.46 g) under ice-cooling, and the mixture was stirred at room temperature for 10 min. A saturated aqueous sodium hydrogencarbonate solution was added to the reaction mixture, and the mixture was diluted with ethyl acetate. The organic layer was washed with saturated brine, dried (MgSO₄), filtrated and concentrated. The obtained residue was subjected to silica gel column chromatography, and eluted with ethyl acetate-hexane (hexane alone to 2:3, v/v) to give a pale-yellow oil.

To a solution of the obtained oil in tetrahydrofuran (30 ml), tert-butanol (24 ml) and water (6 ml) were added sodium dihydrogen phosphate (803 mg), sodium chlorite (601 mg) and 2-methyl-2-butene (6.5 ml) at room temperature, and the mixture was stirred at room temperature for 20 min. A 10% aqueous sodium hydrogen sulfite solution and 1N hydrochloric acid were added to the reaction mixture, and the mixture was diluted with ethyl acetate. The organic layer was washed with saturated brine, dried (MgSO₄), filtrated and concentrated. The obtained residue was subjected to silica gel column chromatography, and eluted with ethyl acetate-hexane (1:9-7:3, v/v) to give 3-[2-{[3-chloro-5-(trifluoromethyl)pyridin-2-yl]oxy}-4-(2-methoxyethoxy)phenyl]butanoic acid (2.51 g, yield: 92%) as a colorless oil.

¹H-NMR (300 MHz, CDCl₃) δ:1.25 (3H, d, J=6.8 Hz), 2.44-2.54 (1H, m), 2.61-2.81 (1H, m), 3.27-3.39 (1H, m), 3.43 (3H, s), 3.65-3.78 (2H, m), 3.99-4.14 (2H, m), 6.65 (1H, d, J=2.6 Hz), 6.85 (1H, dd, J=8.7, 2.4 Hz), 7.12-7.24 (1H, m), 7.98 (1H, d, J=2.3 Hz), 8.22-8.28 (1H, m).

### Reference Example 46

A mixture of ethyl 3-[1-(2,4-difluorobenzyl)-3-isopropoxy-1H-pyrazol-5-yl]propanoate (0.16 g), a 1N aqueous sodium hydroxide solution (4.0 ml), tetrahydrofuran (4.0 ml) and ethanol (4.0 ml) was stirred at 60° C. for 30 min, 1N hydrochloric acid (20 ml) was added, and the mixture was extracted with ethyl acetate. The ethyl acetate layer was washed with saturated brine, dried (MgSO₄) and concentrated to give 3-[1-(2,4-difluorobenzyl)-3-isopropoxy-1H-pyrazol-5-yl]propanoic acid (140 mg, yield: 95%) as a white solid.

¹H-NMR (300 MHz, CDCl₃) δ:1.32 (6H, d, J=6.3 Hz), 2.63 (2H, t, J=7.4 Hz), 2.83 (2H, t, J=7.4 Hz), 4.62-4.74 (1H, m), 5.13 (2H, s), 5.49 (1H, s), 6.74-6.98 (3H, m).

### Reference Example 47

A mixed solution of ethyl 3-[1-(2,4-dichlorobenzyl)-3-isopropoxy-1H-pyrazol-5-yl]propanoate (4.00 g), a 1N aqueous sodium hydroxide solution (18 ml), tetrahydrofuran (20 ml) and methanol (20 ml) was stirred at 50° C. for 2 hr. 1N Hydrochloric acid (19 ml) and brine (30 ml) were added to the reaction mixture, and the mixture was extracted with ethyl acetate (60 ml×2). The organic layer was washed with brine, dried (MgSO₄), filtrated and concentrated. The obtained crude crystals were recrystallized from hexane-ethyl acetate to give 3-[1-(2,4-dichlorobenzyl)-3-isopropoxy-1H-pyrazol-5-yl]propanoic acid (3.54 g, yield: 82%) as colorless crystals. melting point 114-115° C.

### Reference Example 48

A mixture of ethyl 3-[1-(2,4-dichlorobenzyl)-3-isopropyl-1H-pyrazol-5-yl]propanoate (1.00 g), a 1N aqueous sodium hydroxide solution (5.0 ml), tetrahydrofuran (20 ml) and ethanol (20 ml) was stirred at 50° C. for 1 hr. The reaction mixture was concentrated, 1N hydrochloric acid (30 ml) was added, and the mixture was extracted with ethyl acetate. The ethyl acetate layer was washed with saturated brine, dried (MgSO₄), and concentrated. The residue was crystallized from ethyl acetate-hexane to give 3-[1-(2,4-dichlorobenzyl)-3-isopropyl-1H-pyrazol-5-yl]propanoic acid (680 mg, yield: 74%) as colorless crystals. melting point 105-108° C.

### Reference Example 49

A mixture of ethyl (2E)-3-[1-(2,4-dichlorobenzyl)-3-isopropoxy-1H-pyrazol-5-yl]acrylate (950 mg), a 1N aqueous sodium hydroxide solution (8.0 ml), tetrahydrofuran (20 ml) and ethanol (20 ml) was stirred at 50° C. for 1 hr. The reaction mixture was concentrated, 1N hydrochloric acid (30 ml) was added, and the mixture was extracted with ethyl acetate. The ethyl acetate layer was washed with saturated brine, dried (MgSO₄), and concentrated. The residue was crystallized from ethyl acetate-hexane to give (2E)-3-[1-(2,4-dichlorobenzyl)-3-isopropoxy-1H-pyrazol-5-yl]acrylic acid (790 mg, yield: 90%) as colorless crystals. melting point 170-171° C.

### Reference Example 50

A mixture of ethyl (2E)-3-[1-(2,4-dichlorobenzyl)-3-isopropyl-1H-pyrazol-5-yl]acrylate (600 mg), a 1N aqueous sodium hydroxide solution (5.0 ml), tetrahydrofuran (15 ml) and ethanol (15 ml) was stirred at 50° C. for 1 hr. The reaction mixture was concentrated, 1N hydrochloric acid (30 ml) was added, and the mixture was extracted with ethyl acetate. The ethyl acetate layer was washed with saturated brine, dried (MgSO₄), and concentrated. The residue was crystallized from ethyl acetate-hexane to give (2E)-3-[1-(2,4-dichlorobenzyl)-3-isopropyl-1H-pyrazol-5-yl]acrylic acid (420 mg, yield: 75%) as colorless crystals. melting point 174-175° C.

### Reference Example 51

A mixture of ethyl 3-{1-[2-chloro-4-(trifluoromethyl)benzyl]-3-isopropyl-1H-pyrazol-5-yl}propanoate (1.90 g), a 1N aqueous sodium hydroxide solution (10 ml), tetrahydrofuran (30 ml) and ethanol (30 ml) was stirred at 50° C. for 1 hr. The reaction mixture was concentrated, 1N hydrochloric acid (50 ml) was added, and the mixture was extracted with ethyl acetate. The ethyl acetate layer was washed with saturated brine, dried (MgSO₄), and concentrated. The residue was crystallized from ethyl acetate-hexane to give 3-{1-[2-chloro-4-(trifluoromethyl)benzyl]-3-isopropyl-1H-pyrazol-5-yl}propanoic acid (1.20 g, yield: 68%) as colorless crystals. melting point 117-119° C.

### Reference Example 52

A mixture of ethyl 3-[3-butoxy-1-(2,4-dichlorobenzyl)-1H-pyrazol-5-yl]propanoate (2.07 g), a 1N aqueous sodium hydroxide solution (13 ml), tetrahydrofuran (30 ml) and ethanol (30 ml) was stirred at 50° C. for 1 hr. The reaction mixture was concentrated, 1N hydrochloric acid (50 ml) was added, and the mixture was extracted with ethyl acetate. The ethyl acetate layer was washed with saturated brine, dried (MgSO₄), and concentrated. The residue was crystallized from ethyl acetate-hexane to give 3-[3-butoxy-1-(2,4-dichlorobenzyl)-1H-pyrazol-5-yl]propanoic acid (1.70 g, yield: 88%) as colorless crystals. melting point 123-124° C.

### Reference Example 53

A mixture of ethyl 3-[3-butoxy-1-(2,4-dichlorobenzyl)-1H-pyrazol-5-yl]acrylate (4.00 g), a 1N aqueous sodium hydroxide solution (20 ml), tetrahydrofuran (40 ml) and ethanol (40 ml) was stirred at 50° C. for 1 hr. The reaction mixture was concentrated, 1N hydrochloric acid (100 ml) was added, and the mixture was extracted with ethyl acetate. The ethyl acetate layer was washed with saturated brine, dried (MgSO₄), and concentrated. The residue was crystallized from ethyl acetate-hexane to give 3-[3-butoxy-1-(2,4-dichlorobenzyl)-1H-pyrazol-5-yl]acrylic acid (2.82 g, yield: 76%) as colorless crystals melting point 176-179° C.

### Reference Example 54

To a mixture of potassium tert-butoxide (132 g) and tetrahydrofuran (600 ml) was added dropwise a mixture of acetophenone (100 g) and diethyl oxalate (123 g), and the mixture was stirred at room temperature for 15 hr. Acetic acid (119 ml) and hydrazine monohydrate (45.8 g) were added to the reaction mixture, and the mixture was stirred with heating under reflux for 2 hr. After cooling to room temperature, the reaction mixture was concentrated, and the residue was partitioned between water and ethyl acetate. The ethyl acetate layer was washed with saturated aqueous sodium carbonate solution and saturated brine, dried (MgSO₄), and concentrated. The residue was crystallized from diisopropyl ether to give ethyl 3-phenyl-1H-pyrazole-5-carboxylate (145 g, yield: 81%) as a brown solid.

¹H-NMR (300 MHz, CDCl₃) δ:1.36 (3H, t, J=7.2 Hz), 4.36 (2H, q, J=7.2 Hz), 7.10 (1H, s), 7.30-7.46 (3H, m), 7.71-7.80 (2H, m), 11.86 (1H, brs).

### Reference Example 55

A mixture of ethyl 3-phenyl-1H-pyrazole-5-carboxylate (70.0 g), 2,4-dichlorobenzyl chloride (69.6 g), potassium carbonate (53.7 g) and N,N-dimethylformamide (400 ml) was stirred at room temperature for 15 hr, the reaction mixture was concentrated, and the residue was partitioned between water and ethyl acetate. The ethyl acetate layer was washed with water and saturated brine, dried (MgSO₄), and concentrated. The residue was crystallized from ethyl acetate-hexane to give ethyl 1-(2,4-dichlorobenzyl)-3-phenyl-1H-pyrazole-5-carboxylate (61.0 g, yield: 50%) as brown crystals melting point 104-107° C.

### Reference Example 56

To a solution of ethyl 1-(2,4-dichlorobenzyl)-3-phenyl-1H-pyrazole-5-carboxylate (60.0 g) in tetrahydrofuran (400 ml) was added a 1.5 M solution (267 ml) of diisobutylaluminum hydride in toluene at 0° C. The mixture was stirred at room temperature for 2 hr and methanol was added to quench the reaction. The reaction mixture was poured into a 10% aqueous Rochelle salt solution, and the mixture was stirred and extracted with ethyl acetate. The extract was washed with water and saturated brine, dried (MgSO₄), and concentrated. The residue was crystallized from ethyl acetate-hexane to give [1-(2,4-dichlorobenzyl)-3-phenyl-1H-pyrazol-5-yl]methanol (48.9 g, yield: 92%) as pale-brown crystals. melting point 140-141° C.

### Reference Example 57

Under a nitrogen atmosphere, to a solution of oxalyl chloride (15.2 g) in dichloromethane (100 ml) was added dimethyl sulfoxide (28.1 g) at −78° C., and the mixture was stirred for 5 min. A solution of [1-(2,4-dichlorobenzyl)-3-phenyl-1H-pyrazol-5-yl]methanol (20.0 g) in dichloromethane (300 ml) was added. After stirring at −78° C. for 1 hr, triethylamine (30.3 g) was added, and the reaction mixture was warmed to room temperature and further stirred for 1 hr. The reaction mixture was concentrated, and the residue was partitioned between water and ethyl acetate. The ethyl acetate layer was washed with 1N hydrochloric acid and saturated brine, dried (MgSO₄), and concentrated. The residue was crystallized from ethyl acetate-hexane to give 1-(2,4-dichlorobenzyl)-3-phenyl-1H-pyrazole-5-carbaldehyde (13.3 g, yield: 67%) as pale-yellow crystals. melting point 135-137° C.

### Reference Example 58

Under ice-cooling, to a solution of ethyl diethylphosphonoacetate (10.2 g) in N,N-dimethylformamide (50 ml) was added sodium hydride (60% in oil, 1.57 g), and the mixture was stirred at room temperature for 30 min. The reaction mixture was ice-cooled, a solution of 1-(2,4-dichlorobenzyl)-3-phenyl-1H-pyrazole-5-carbaldehyde (10.0 g) in tetrahydrofuran (60 ml) was added, and the mixture was stirred for 1 hr. A saturated aqueous ammonium chloride solution was added to quench the reaction. The reaction mixture was concentrated, and the residue was partitioned between water and ethyl acetate. The ethyl acetate layer was washed with water and saturated brine, dried (MgSO₄), and concentrated. The residue was crystallized from ethyl acetate-hexane to give ethyl (2E)-3-[1-(2,4-dichlorobenzyl)-3]phenyl-1H-pyrazol-5-yl]acrylate (11.5 g, yield: 95%) as colorless crystals. melting point 123-124° C.

### Reference Example 59

A mixture of ethyl (2E)-3-[1-(2,4-dichlorobenzyl)-3-phenyl-1H-pyrazol-5-yl]acrylate (7.00 g), 5% palladium-carbon (860 mg) and tetrahydrofuran (100 ml) was hydrogenated at room temperature under atmospheric pressure. The reaction mixture was filtrated, and the filtrate was concentrated. The residue was crystallized from ethyl acetate-hexane to give ethyl 3-[1-(2,4-dichlorobenzyl)-3-phenyl-1H-pyrazol-5-yl]propanoate (4.18 g, yield: 60%) as colorless crystals. melting point 91-93° C.

### Reference Example 60

A mixture of ethyl 3-[1-(2,4-dichlorobenzyl)-3-phenyl-1H-pyrazol-5-yl]propanoate (5.22 g), a 1N aqueous sodium hydroxide solution (20 ml), tetrahydrofuran. (50 ml) and ethanol (50 ml) was stirred at 50° C. for 1 hr. The reaction mixture was concentrated, 1N hydrochloric acid (100 ml) was added, and the mixture was extracted with ethyl acetate. The ethyl acetate layer was washed with saturated brine, dried (MgSO₄), and concentrated. The residue was crystallized from ethyl acetate-hexane to give 3-[1-(2,4-dichlorobenzyl)-3-phenyl-1H-pyrazol-5-yl]propanoic acid (4.28 g, yield: 88%) as colorless crystals. melting point 160-16:1° C.

### Reference Example 61

To a solution of ethyl 3-[1-(2,4-dichlorobenzyl)-3-hydroxy-1H-pyrazol-5-yl]propanoate (2.00 g), 2-methoxyethanol (887 mg) and tributylphosphine (2.37 g) in tetrahydrofuran (150 ml) was added 1,1′-(azodicarbonyl)dipiperidine (2.95 g), and the mixture was stirred at room temperature for 15 hr and concentrated. Diisopropyl ether was added to the residue, the resulting insoluble material was filtered off, and the filtrate was concentrated. The residue was subjected to silica gel column chromatography, and eluted with ethyl acetate-hexane (1:24-3:1, v/v) to give ethyl 3-[1-(2,4-dichlorobenzyl)-3-(2-methoxyethoxy)-1H-pyrazol-5-yl]propanoate (1.70 g, yield: 73%) as a colorless oil.

A mixture of ethyl 3-[1-(2,4-dichlorobenzyl)-3-(2-methoxyethoxy)-1H-pyrazol-5-yl]propanoate (1.70 g), a 1N aqueous sodium hydroxide solution (10 ml), tetrahydrofuran (25 ml) and ethanol (25 ml) was stirred at 50° C. for 1 hr. The reaction mixture was concentrated, 1N hydrochloric acid (100 ml) was added, and the mixture was extracted with ethyl acetate. The ethyl acetate layer was washed with saturated brine, dried (MgSO₄), and concentrated. The residue was crystallized from ethyl acetate-hexane to give 3-[1-(2,4-dichlorobenzyl)-3-(2-methoxyethoxy)-1H-pyrazol-5-yl]propanoic acid (1.48 g, yield: 94%) as colorless crystals. melting point 111-113° C.

### Reference Example 62

To a solution of ethyl 3-[1-(2,4-dichlorobenzyl)-3-hydroxy-1H-pyrazol-5-yl]propanoate (1.00 g), benzyl alcohol (630 mg) and tributylphosphine (1.18 g) in tetrahydrofuran (70 ml) was added 1,1′-(azodicarbonyl)dipiperidine (1.47 g), and the mixture was stirred at room temperature for 15 hr and concentrated. Diisopropyl ether was added to the residue, the resulting insoluble material was filtered off, and the filtrate was concentrated. The residue was subjected to silica gel column chromatography, and eluted with ethyl acetate-hexane (1:19-1:4, v/v) to give ethyl 3-[3-(benzyloxy)-1-(2,4-dichlorobenzyl)-1H-pyrazol-5-yl]propanoate (510 mg, yield: 40%) as a colorless oil.

A mixture of ethyl 3-[3-(benzyloxy)-1-(2,4-dichlorobenzyl)-1H-pyrazol-5-yl]propanoate (510 mg), a 1N aqueous sodium hydroxide solution (3.0 ml), tetrahydrofuran (8.0 ml) and ethanol (8.0 ml) was stirred at 50° C. for 1 hr. The reaction mixture was concentrated, 1N hydrochloric acid (50 ml) was added, and the mixture was extracted with ethyl acetate. The ethyl acetate layer was washed with saturated brine, dried (MgSO₄), and concentrated. The residue was crystallized from ethyl acetate-hexane to give 3-[3-(benzyloxy)-1-(2,4-dichlorobenzyl)-1H-pyrazol-5-yl]propanoic acid (420 mg, yield: 88%) as colorless crystals. melting point 98-101° C.

### Reference Example 63

A mixture of methyl 3-hydroxypyrazole-5-carboxylate (48.2 g), potassium carbonate (51.6 g), 1-iodobutane (40.5 g) and N,N-dimethylformamide (300 ml) was stirred at room temperature for 12 hr. The reaction mixture was concentrated, and the concentrate was partitioned between water and ethyl acetate. The ethyl acetate layer was washed with water and saturated brine, dried (MgSO₄), and concentrated. The residue was crystallized from hexane to give methyl 3-butoxypyrazole-5-carboxylate (43.6 g, yield: 64%, containing isomer by about 10%) as colorless crystals.

¹H-NMR (300 MHz, CDCl₃) δ:0.96 (3H, t, J=7.2 Hz), 1.41-1.54 (2H, m), 1.71-1.81 (2H, m), 3.91 (3H, s), 4.16 (2H, t, J=6.6 Hz), 6.21 (1H, s), 9.90-10.20 (1H, brs).

### Reference Example 64

A mixture of methyl 3-butoxypyrazole-5-carboxylate (5.00 g), 4-(trifluoromethyl)benzyl bromide (7.22 g), potassium carbonate (5.22 g) and N,N-dimethylformamide (50 ml) was stirred at room temperature for 15 hr. The reaction mixture was poured into water to partition the mixture between water and ethyl acetate. The ethyl acetate layer was washed with water and saturated brine, dried (MgSO₄), and concentrated. The residue was subjected to silica gel column chromatography, and eluted with ethyl acetate-hexane (1:24-1:9, v/v) to give methyl 3-butoxy-1-[4-(trifluoromethyl)benzyl]-1H-pyrazole-5-carboxylate (5.79 g, yield: 64%, containing isomer by about 10%) as a colorless oil.

¹H-NMR (300 MHz, CDCl₃) δ:0.96 (3H, t, J=7.2 Hz), 1.41-1.55 (2H, m), 1.68-1.82 (2H, m), 3.83 (3H, s), 4.12 (2H, t, J=6.6 Hz), 5.65 (2H, s), 6.25 (1H, s), 7.33 (2H, d, J=8.1 Hz), 7.55 (2H, d, J=8.1 Hz).

### Reference Example 65

A mixture of methyl 3-butoxy-1-[4-(trifluoromethyl)benzyl]-1H-pyrazole-5-carboxylate (5.79 g), a 1N aqueous sodium hydroxide solution (30 ml), tetrahydrofuran (60 ml) and ethanol (60 ml) was stirred at 50° C. for 1 hr. The reaction mixture was concentrated, 1N hydrochloric acid (200 ml) was added, and the mixture was extracted with ethyl acetate. The ethyl acetate layer was washed with saturated brine, dried (MgSO₄), and concentrated. The residue was crystallized from ethyl acetate-hexane to give 3-butoxy-1-[4-(trifluoromethyl)benzyl]-1H-pyrazole-5-carboxylic acid (3.33 g, yield: 60%) as colorless crystals. melting point 128-129° C.

### Reference Example 66

A mixture of N,O-dimethylhydroxylamine hydrochloride (1.13 g), triethylamine (1.17 g) and N,N-dimethylformamide (30 ml) was stirred at room temperature for 30 min, 3-butoxy-1-[4-(trifluoromethyl)benzyl]-1H-pyrazole-5-carboxylic acid (3.30 g), 1-ethyl-3-(3-dimethylaminopropyl)carbodiimide hydrochloride (2.22 g) and 1-hydroxybenzotriazole monohydrate (1.78 g) were added, and the mixture was stirred at room temperature for 15 hr. The reaction mixture was poured into water, and the mixture was extracted with ethyl acetate. The extract was washed with 1N hydrochloric acid, saturated aqueous sodium hydrogencarbonate, water and saturated brine, dried (MgSO₄), and concentrated. The residue was subjected to silica gel column chromatography, and eluted with ethyl acetate-hexane (1:19-1:2, v/v) to give 3-butoxy-N-methoxy-N-methyl-1-[4-(trifluoromethyl)benzyl]-1H-pyrazole-5-carboxamide (3.01 g, yield: 81%) as a colorless oil.

¹H-NMR (300 MHz, CDCl₃) δ:0.97 (3H, t, J=7.2 Hz), 1.41-1.55 (2 H, m), 1.70-1.81 (2H, m), 3.27 (3H, s), 3.55 (3H, s), 4.15 (2H, t, J=6.6 Hz), 5.59 (2H, s), 6.22 (1H, s), 7.30 (2H, d, J=8.1 Hz), 7.53 (2H, d, J=8.1 Hz).

### Reference Example 67

To a solution of 3-butoxy-N-methoxy-N-methyl-1-[4-(trifluoromethyl)benzyl]-1H-pyrazole-5-carboxamide (3.00 g) in tetrahydrofuran (100 ml) was added a 1.5 M solution (11.7 ml) of diisobutylaluminum hydride in toluene at 0° C., and the mixture was stirred for 1 hr. Methanol was added to quench the reaction. The reaction mixture was poured into a 10% aqueous Rochelle salt solution, and the mixture was stirred and extracted with diethyl ether. The extract was washed with saturated brine, dried (MgSO₄), and concentrated. The residue was subjected to silica gel column chromatography, and eluted with ethyl acetate-hexane (1:19-3:17, v/v) to give 3-butoxy-1-[4-(trifluoromethyl)benzyl]-1H-pyrazole-5-carbaldehyde (2.31 g, yield: 91%) as a colorless oil.

¹H-NMR (300 MHz, CDCl₃) δ:0.97 (3H, t, J=7.2 Hz), 1.40-1.55 (2H, m), 1.70-1.82 (2H, m), 4.16 (2H, t, J=6.5 Hz), 5.61 (2H, s), 6.29 (1H, s), 7.37 (2H, d, J=8.6 Hz), 7.55 (2H, d, J=8.6 Hz), 9.71 (1H, s).

### Reference Example 68

Under ice-cooling, to a solution of ethyl diethylphosphonoacetate (2.38 g) in N,N-dimethylformamide (20 ml) was added sodium hydride (60% in oil, 368 mg), and the mixture was stirred at room temperature for 30 min. The reaction mixture was ice-cooled, a solution of 3-butoxy-1-[4-(trifluoromethyl)benzyl]-1H-pyrazole-5-carbaldehyde (2.31 g) in tetrahydrofuran (15 ml) was added, and the mixture was stirred for 1 hr. The mixture was warmed to room temperature and further stirred for 1 hr. A saturated aqueous ammonium chloride solution was added to quench the reaction. The reaction mixture was concentrated, and the residue was partitioned between water and ethyl acetate. The ethyl acetate layer was washed with water and saturated brine, dried (MgSO₄), and concentrated. The residue was subjected to silica gel column chromatography, and eluted with ethyl acetate-hexane (1:19-3:17, v/v) to give ethyl (2E)-3-{3-butoxy-1-[4-(trifluoromethyl)benzyl]-1H-pyrazol-5-yl}acrylate (2.02 g, yield: 72%) as a colorless oil.

¹H-NMR (300 MHz, CDCl₃) δ:0.96 (3H, t, J=7.4 Hz), 1.30 (3H, t, J=7.2 Hz), 1.40-1.55 (2H, m), 1.69-1.81 (2H, m), 4.12 (2H, t, J=6.6 Hz), 4.23 (2H, q, J=7.2 Hz), 5.33 (2H, s), 6.01 (1H, s), 6.28 (1H, d, J=15.8 Hz), 7.22 (2H, d, J=8.1 Hz), 7.41 (1H, d, J=15.8 Hz), 7.57 (2H, d, J=8.1 Hz).

### Reference Example 69

A mixture of ethyl (2E)-3-{3-butoxy-1-[4-(trifluoromethyl)benzyl]-1H-pyrazol-5-yl}acrylate (1.00 g), a 1N aqueous sodium hydroxide solution (5.0 ml), tetrahydrofuran (10 ml) and ethanol (10 ml) was stirred at 50° C. for 1 hr. The reaction mixture was concentrated, 1N hydrochloric acid (50 ml) was added, and the mixture was extracted with ethyl acetate. The ethyl acetate layer was washed with saturated brine, dried (MgSO₄), and concentrated. The residue was crystallized from ethyl acetate-hexane to give (2E)-3-{3-butoxy-1-[4-(trifluoromethyl)benzyl]-1H-pyrazol-5-yl}acrylic acid (760 mg, yield: 82%) as colorless crystals. melting point 153-155° C.

### Reference Example 70

A mixture of ethyl (2E)-3-{3-butoxy-1-[4-(trifluoromethyl)benzyl]-1H-pyrazol-5-yl}acrylate (1.01 g), 5% palladium-carbon (250 mg) and tetrahydrofuran (20 ml) was hydrogenated at room temperature under atmospheric pressure. The reaction mixture was filtrated, and the filtrate was concentrated to give ethyl 3-{3-butoxy-1-[4-(trifluoromethyl)benzyl]-1H-pyrazol-5-yl}propanoate (950 mg, yield: 94%) as a colorless oil.

A mixture of, ethyl 3-{3-butoxy-1-[4-(trifluoromethyl)benzyl]-1H-pyrazol-5-yl}propanoate (950 mg), a 1N aqueous sodium hydroxide solution (5.0 ml), tetrahydrofuran (10 ml) and ethanol (10 ml) was stirred at 50° C. for 1 hr. The reaction mixture was concentrated, 1N hydrochloric acid (100 ml) was added, and the mixture was extracted with ethyl acetate. The ethyl acetate layer was washed with saturated brine, dried (MgSO₄), and concentrated. The residue was crystallized from ethyl acetate-hexane to give 3-{3-butoxy-1-[4-(trifluoromethyl)benzyl]-1H-pyrazol-5-yl}propanoic acid (720 mg, yield: 66%) as colorless crystals. melting point 116-118° C.

### Reference Example 71

A mixture of ethyl 3-[1-(2,4-dichlorobenzyl)-3-hydroxy-1H-pyrazol-5-yl]propanoate (2.00 g), tert-butyl bromoacetate (1.37 g), potassium carbonate (1.21 g) and N,N-dimethylformamide (30 ml) was stirred at room temperature for 15 hr. The reaction mixture was concentrated, and the concentrate was partitioned between water and ethyl acetate. The ethyl acetate layer was washed with saturated brine, dried (MgSO₄), and concentrated. The residue was subjected to silica gel column chromatography, and eluted with ethyl acetate-hexane (1:19-3:7, v/v) to give ethyl 3-[3-(2-tert-butoxy-2-oxoethoxy)-1-(2,4-dichlorobenzyl)-1H-pyrazol-5-yl]propanoate (2.03 g, yield: 76%) as a colorless solid.

A mixture of ethyl 3-[3-(2-tert-butoxy-2-oxoethoxy)-1-(2,4-dichlorobenzyl)-1H-pyrazol-5-yl]propanoate and trifluoroacetic acid (20 ml) was stirred at room temperature for 1 hr. The reaction mixture was concentrated. The residue was crystallized from ethyl acetate-hexane to give {[1-(2,4-dichlorobenzyl)-5-(3-ethoxy-3-oxopropyl)-1H-pyrazol-3-yl]oxy}acetic acid (1.65 g, yield: 93%) as colorless crystals melting point 129-131° C.

### Reference Example 72

To a solution of {[1-(2,4-dichlorobenzyl)-5-(3-ethoxy-3-oxopropyl)-1H-pyrazol-3-yl]oxy}acetic acid (400 mg) in tetrahydrofuran (7.0 ml) was added N,N′-carbonyldiimidazole (178 mg), and the mixture was stirred at room temperature for 2 hr. Acetylhydrazine (296 mg) was added, and the mixture was further stirred at room temperature for 3 hr. The reaction mixture was concentrated, and the residue was partitioned between ethyl acetate and water. The ethyl acetate layer was washed with saturated brine, dried (MgSO₄), and concentrated. The residue was crystallized from ethyl acetate-hexane to give ethyl 3-{3-[2-(2-acetylhydrazino)-2-oxoethoxy]-1-(2,4-dichlorobenzyl)-1H-pyrazol-5-yl}propanoate (410 mg, yield: 90%) as colorless crystals. melting point 167-170° C.

### Reference Example 73

Ethyl 3-{3-[2-(2-acetylhydrazino)-2-oxoethoxy]-1-(2,4-dichlbrobenzyl)-1H-pyrazol-5-yl}propanoate (410 mg), diphosphorus pentaoxide (510 mg), hexamethyldisiloxane (1.17 g) and toluene (20 ml) was stirred with heating under reflux for 3 hr. The reaction mixture was poured into water, and the mixture was extracted with ethyl acetate. The extract was washed with saturated aqueous sodium hydrogencarbonate and saturated brine, dried (MgSO₄), and concentrated. The residue was subjected to silica gel column chromatography, and eluted with ethyl acetate-hexane (1:19-2:3, v/v) to give ethyl 3-{1-(2,4-dichlorobenzyl)-3-[(5-methyl-1,3,4-oxadiazol-2-yl)methoxy]-1H-pyrazol-5-yl}propanoate (270 mg, yield: 69%) as a colorless oil.

¹H-NMR (300 MHz, CDCl₃) δ:1.25 (3H, t, J=7.2 Hz), 2.54 (3H, s), 2.55-2.62 (2H, m), 2.74-2.83 (2H, m), 4.12 (2H, q, J=7.2 Hz), 5.20 (2H, s), 5.33 (2H, s), 5.62 (1H, s), 6.56 (1H, d, J=8.4 Hz), 7.16 (1H, dd, J=2.1, 8.4 Hz), 7.39 (1H, d, J=2.1 Hz)

### Reference Example 74

Under ice-cooling, to a solution of ethyl 3-{1-(2,4-dichlorobenzyl)-3-[(5-methyl-1,3,4-oxadiazol-2-yl)methoxy]-1H-pyrazol-5-yl}propanoate (270 mg) in tetrahydrofuran (4.0 ml) was added a solution of lithium hydroxide monohydrate (28 mg) in water (1.0 ml). The mixture was stirred for 20 min, warmed to room temperature, and further stirred for 20 min. A solution of lithium hydroxide monohydrate (28 mg) in water (1.0 ml) was added to the reaction mixture, and the mixture was stirred at room temperature for 20 min. A 10% citric acid aqueous solution (50 ml) was added to quench the reaction. The reaction mixture was concentrated under reduced pressure to evaporate tetrahydrofuran, and the mixture was extracted with ethyl acetate. The extract was washed with saturated brine, dried (MgSO₄) and concentrated to give 3-{1-(2,4-dichlorobenzyl)-3-[(5-methyl-1,3,4-oxadiazol-2-yl)methoxy]-1H-pyrazol-5-yl}propanoic acid (250 mg) as a white solid.

¹H-NMR (300 MHz, CDCl₃) δ:2.55 (3H, s), 2.59-2.67 (2H, m), 2.74-2.82 (2H, m), 5.19 (2H, s), 5.34 (2H, s), 6.55 (1H, d, J=8.1 Hz), 7.15 (1H, dd, J=2.1, 8.1 Hz), 7.38 (1H, d, J=2.1 Hz), 11.02 (1H, brs).

### Reference Example 75

A mixture of methyl 3-butoxypyrazole-5-carboxylate (7.28 g), 2-chloro-1-(chloromethyl)-4-(trifluoromethyl)benzene (38.9 g), potassium carbonate (7.61 g) and N,N-dimethylformamide (60 ml) was stirred at room temperature for 15 hr. The reaction mixture was poured into water, and the mixture was partitioned between water and ethyl acetate. The ethyl acetate layer was washed with water and saturated brine, dried (MgSO₄), and concentrated. The residue was subjected to silica gel column chromatography, and eluted with ethyl acetate-hexane (1:24-1:10, v/v) to give methyl 3-butoxy-1-[2-chloro-4-(trifluoromethyl)benzyl]-1H-pyrazole-5-carboxylate (9.79 g, yield: 68%) as a colorless oil.

¹H-NMR (300 MHz, CDCl₃) δ:0.96 (3H, t, J=7.2 Hz), 1.39-1.54 (2H, m), 1.67-1.82 (2H, m), 3.82 (3H, s), 4.13 (2H, t, J=6.6 Hz), 5.76 (2H, s), 6.32 (1H, s), 6.66 (1H, d, J=8.1 Hz), 7.39 (1H, dd, J=1.2, 8.1 Hz), 7.64 (1H, d, J=1.2 Hz).

### Reference Example 76

A mixture of methyl 3-butoxy-1-[2-chloro-4-(trifluoromethyl)benzyl]-1H-pyrazole-5-carboxylate (9.79 g), a 1N aqueous sodium hydroxide solution (45 ml), tetrahydrofuran (90 ml) and ethanol (90 ml) was stirred at 50° C. for 1 hr. The reaction mixture was concentrated, 1N hydrochloric acid (200 ml) was added, and the mixture was extracted with ethyl acetate. The ethyl acetate layer was washed with saturated brine, dried (MgSO₄), and concentrated. The residue was crystallized from ethyl acetate-hexane to give 3-butoxy-1-[2-chloro-4-(trifluoromethyl)benzyl]-1H-pyrazole-5-carboxylic acid. (7.88 g, yield: 83%) as colorless crystals. melting point 158-160° C.

### Reference Example 77

A mixture of N,O-dimethylhydroxylamine hydrochloride (2.42 g), triethylamine (2.51 g) and N,N-dimethylformamide (100 ml) was stirred at room temperature for 30 min, 3-butoxy-1-[2-chloro-4-(trifluoromethyl)benzyl]-1H-pyrazole-5-carboxylic acid (7.79 g), 1-ethyl-3-(3-dimethylaminopropyl)carbodiimide hydrochloride (4.75 g) and 1-hydroxybenzotriazole monohydrate (3.80 g) were added, and the mixture was stirred at room temperature for 15 hr. The reaction mixture was poured into water, and the mixture was extracted with ethyl acetate. The extract was washed with 1N hydrochloric acid, saturated aqueous sodium hydrogencarbonate, water and saturated brine, dried (MgSO₄), and concentrated. The residue was subjected to silica gel column chromatography, and eluted with ethyl acetate-hexane (1:19-1:2, v/v) to give 3-butoxy-1-[2-chloro-4-(trifluoromethyl)benzyl]-N-methoxy-N-methyl-1H-pyrazole-5-carboxamide (8.69 g, yield: 100%) as a colorless oil.

¹H-NMR (300 MHz, CDCl₃) δ:0.96 (3H, t, J=7.4 Hz), 1.40-1.55 (2H, m), 1.70-1.81 (2H, m), 3.28 (3H, s), 3.66 (3H, s), 4.15 (2H, t, J=6.6 Hz), 5.68 (2H, s), 6.29 (1H, s), 6.70 (1H, d, J=8.1 Hz), 7.38 (1H, d, J=8.1 Hz), 7.61 (1H, S)

### Reference Example 78

To a solution of 3-butoxy-1-[2-chloro-4-(trifluoromethyl)benzyl]-N-methoxy-N-methyl-1H-pyrazole-5-carboxamide (8.69 g) in tetrahydrofuran (250 ml) was added a 1.5 M solution (21 ml) of diisobutylaluminum hydride in toluene at 0° C., and the mixture was stirred for 1 hr. Methanol was added to quench the reaction. The reaction mixture was poured into a 10% aqueous Rochelle salt solution, and the mixture was stirred and extracted with diethyl ether. The extract was washed with saturated brine, dried (MgSO₄), and concentrated. The residue was subjected to silica gel column chromatography, and eluted with ethyl acetate-hexane (1:19-1:4, v/v) to give 3-butoxy-1-[2-chloro-4-(trifluoromethyl)benzyl]-1H-pyrazole-5-carbaldehyde (6.31 g, yield: 84%) as a pale-yellow oil.

¹H-NMR (300 MHz, CDCl₃) δ:0.96 (3H, t, J=7.2 Hz), 1.40-1.54 (2H, m), 1.69-1.81 (2H, m), 4.16 (2H, t, J=6.6 Hz), 5.73 (2H, s), 6.36 (1H, s), 6.70 (1H, d, J=7.8 Hz), 7.39 (1H, dd, J=1.2, 7.8 Hz), 7.65 (1H, d, J=7.8 Hz), 9.75 (1H, s).

### Reference Example 79

Under ice-cooling, to a solution of ethyl diethylphosphonoacetate (3.26 g) in N,N-dimethylformamide (25 ml) was added sodium hydride (60% in oil, 504 mg), and the mixture was stirred at room temperature for 30 min. The reaction mixture was ice-cooled, a solution of 3-butoxy-1-[2-chloro-4-(trifluoromethyl)benzyl]-1H-pyrazole-5-carbaldehyde (3.50 g) in tetrahydrofuran (22 ml) was added, and the mixture was stirred for 1 hr. The mixture was warmed to room temperature and further stirred for 1 hr. A saturated aqueous ammonium chloride solution was added to quench the reaction. The reaction mixture was concentrated, and the residue was partitioned between water and ethyl acetate. The ethyl acetate layer was washed with water and saturated brine, dried (MgSO₄), and concentrated. The residue was subjected to silica gel column chromatography, and eluted with ethyl acetate-hexane (1:49-1:9, v/v) to give ethyl (2E)-3-{3-butoxy-1-[2-chloro-4-(trifluoromethyl)benzyl]-1H-pyrazol-5-yl}acrylate (1.74 g, yield: 42%) as a colorless oil.

¹H-NMR (300 MHz, CDCl₃) δ:0.96 (3H, t, J=7.4 Hz), 1.30 (3H, t, J=7.2 Hz), 1.40-1.55 (2H, m), 1.69-1.81 (2H, m), 4.12 (2H, t, J=6.6 Hz), 4.23 (2H, q, J=7.2 Hz), 5.33 (2H, s), 6.01 (1H, s), 6.28 (1H, d, J=15.8 Hz), 7.22 (2H, d, J=8.1 Hz), 7.41 (1H, d, J=15.8 Hz), 7.57 (2H, d, J=8.1 Hz).

### Reference Example 80

A mixture of ethyl (2E)-3-{3-butoxy-1-[2-chloro-4-(trifluoromethyl)benzyl]-1H-pyrazol-5-yl}acrylate (1.86 g), a 1N aqueous sodium hydroxide solution (10 ml), tetrahydrofuran (20 ml) and ethanol (20 ml) was stirred at 50° C. for 1 hr. The reaction mixture was concentrated, 1N hydrochloric acid (50 ml) was added, and the mixture was extracted with ethyl acetate. The ethyl acetate layer was washed with saturated brine, dried (MgSO₄), and concentrated. The residue was crystallized from ethyl acetate-hexane to give (2E)-3-{3-butoxy-1-[2-chloro-4-(trifluoromethyl)benzyl]-1H-pyrazol-5-yl}acrylic acid (730 mg, yield: 42%) as colorless crystals melting point 172-174° C.

### Reference Example 81

A mixture of ethyl (2E)-3-{3-butoxy-1-[2-chloro-4-(trifluoromethyl)benzyl]-1H-pyrazol-5-yl}acrylate (1.70 g), 5% palladium-carbon (360 mg) and tetrahydrofuran (30 ml) was hydrogenated at room temperature under atmospheric pressure. The reaction mixture was filtrated, and the filtrate was concentrated to give ethyl 3-{3-butoxy-1-[2-chloro-4-(trifluoromethyl)benzyl]-1H-pyrazol-5-yl}propanoate (1.67 g, yield: 98%) as a pale-yellow oil.

A mixture of ethyl 3-{3-butoxy-1-[2-chloro-4-(trifluoromethyl)benzyl]-1H-pyrazol-5-yl}propanoate (1.67 g), a 1N aqueous sodium hydroxide solution (5.0 ml), tetrahydrofuran (10 ml) and ethanol (10 ml) was stirred at 50° C. for 1 hr. The reaction mixture was concentrated, 1N hydrochloric acid (50 ml) was added, and the mixture was extracted with ethyl acetate. The ethyl acetate layer was washed with saturated brine, dried (MgSO₄), and concentrated. The residue was crystallized from ethyl acetate-hexane to give 3-{3-butoxy-1-[2-chloro-4-(trifluoromethyl)benzyl]-1H-pyrazol-5-yl}propanoic acid (1.25 g, yield: 80%) as colorless crystals. melting point 125-126° C.

### Reference Example 82

A mixture of ethyl 3-[1-(2,4-dichlorobenzyl)-3-hydroxy-1H-pyrazol-5-yl]propanoate (2.00 g), 2-(chloromethyl)pyridine (573 mg), potassium carbonate (1.01 g) and N,N-dimethylformamide (20 ml) was stirred at room temperature for 15 hr. The reaction mixture was poured into water, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:19-1:2, v/v) to give ethyl 3-[1-(2,4-dichlorobenzyl)-3-(pyridin-2-ylmethoxy)-1H-pyrazol-5-yl]propanoate (1.00 g, yield: 79%) as a colorless solid.

A mixture of ethyl 3-[1-(2,4-dichlorobenzyl)-3-(pyridin-2-ylmethoxy)-1H-pyrazol-5-yl]propanoate (1.00 g), a 1N aqueous sodium hydroxide solution (5.0 ml), tetrahydrofuran (10 ml) and ethanol (10 ml) was stirred at 50° C. for 1 hr. The reaction mixture was concentrated, water (200 ml) was added, and the precipitated crystals were collected by filtration to give 3-[1-(2,4-dichlorobenzyl)-3-(pyridin-2-ylmethoxy)-1H-pyrazol-5-yl]propanoic acid (0.76 g, yield: 75%) as a white solid.

¹H-NMR (300 MHz, CDCl₃) δ:2.46-2.56 (2H, m), 2.67-2.77 (2H, m), 5.15 (2H, s), 5.20 (2H, s), 5.72 (1H, s), 6.62 (1H, d, J=8.4 Hz), 7.27-7.34 (1H, m), 7.35 (1H, dd, J=2.1, 8.4 Hz), 7.43 (1H, d, J=7.8 Hz), 7.65 (1H, d, J=2.1 Hz), 7.77-7.84 (1H, m), 8.51-8.56 (1H, m), 12.30 (1H, brs).

### Reference Example 83

A mixture of ethyl (2E)-3-{1-[2-chloro-4-(trifluoromethyl)benzyl]-3-hydroxy-1H-pyrazol-5-yl}acrylate (275 mg), bromomethylcyclopropane (560 mg), potassium carbonate (760 mg) and N,N-dimethylformamide (20 ml) was stirred at room temperature for 15 hr. The reaction mixture was concentrated, and the concentrate was partitioned between water and ethyl acetate. The ethyl acetate layer was washed with saturated brine, dried (MgSO₄), and concentrated. The residue was subjected to silica gel column chromatography, eluted with ethyl acetate-hexane (1:19-1:4, v/v), concentrated and crystallized from ethyl acetate-hexane to give ethyl (2E)-3-[1-[2-chloro-4-(trifluoromethyl)benzyl]-3-(cyclopropylmethoxy)-1H-pyrazol-5-yl]acrylate (690 mg, yield: 59%) as colorless crystals.

A mixture of ethyl (2E)-3-[1-[2-chloro-4-(trifluoromethyl)benzyl]-3-(cyclopropylmethoxy)-1H-pyrazol-5-yl]acrylate (400 mg), a 1N aqueous sodium hydroxide solution (3.0 ml), tetrahydrofuran (6.0 ml) and ethanol (6.0 ml) was stirred at 50° C. for 1 hr. The reaction mixture was concentrated, 1N hydrochloric acid (50 ml) was added, and the mixture was extracted with ethyl acetate. The ethyl acetate layer was washed with saturated brine, dried (MgSO₄), and concentrated. The residue was crystallized from ethyl acetate-hexane to give (2E)-3-[1-[2-chloro-4-(trifluoromethyl)benzyl]-3-(cyclopropylmethoxy)-1H-pyrazol-5-yl]acrylic acid (300 mg, yield: 80%) as colorless crystals. melting point 195-196° C.

### Reference Example 84

Ethyl (2E)-3-[1-(2,4-Dichlorobenzyl)-3-(methoxymethoxy)-1H-pyrazol-5-yl]acrylate (2.49 g), concentrated hydrochloric acid (0.15 ml) and methanol (30 ml) were stirred with heating under reflux for 5 hr. The reaction mixture was concentrated, and the residue was crystallized from diisopropyl ether-hexane-ethyl acetate to give ethyl (2E)-3-[1-(2,4-dichlorobenzyl)-3-hydroxy-1H-pyrazol-5-yl]acrylate (2.41 g) as a white solid.

¹H-NMR (300 MHz, CDCl₃) δ:1.31 (3H, t, J=7.2 Hz), 4.23 (2H, q, J=7.2 Hz), 5.28 (2H, s), 5.95 (1H, s), 6.32 (1H, d; J=15.6 Hz), 6.72 (1H, d, J=8.4 Hz), 7.17 (1H, dd, J=2.1, 8.4 Hz), 7.38 (1H, d, J=15.6 Hz), 7.41 (1H, d, J=2.1 Hz).

### Reference Example 85

To a solution of ethyl (2E)-3-[1-(2,4-dichlorobenzyl)-3-hydroxy-1H-pyrazol-5-yl]acrylate (1.20 g), 2-methoxyethanol (533 mg) and tributylphosphine (1.42 g) in tetrahydrofuran (70 ml) was added 1,1′-(azodicarbonyl)dipiperidine (1.77 g), and the mixture was stirred at room temperature for 15 hr and concentrated. Diisopropyl ether was added to the residue, the resulting insoluble material was filtered off, and the filtrate was concentrated. The residue was subjected to silica gel column chromatography, and eluted with ethyl acetate-hexane (1:24-3:1, v/v) to give ethyl (2E)-3-[1-(2,4-dichlorobenzyl)-3-(2-methoxyethoxy)-1H-pyrazol-5-yl]acrylate (850 mg, yield: 61%) as colorless crystals.

A mixture of ethyl (2E)-3-[1-(2,4-dichlorobenzyl)-3-(2-methoxyethoxy)-1H-pyrazol-5-yl]acrylate (850 mg), a 1N aqueous sodium hydroxide solution (5.0 ml), tetrahydrofuran (10 ml) and ethanol (10 ml) was stirred at 50° C. for 1 hr. The reaction mixture was concentrated, 1N hydrochloric acid (50 ml) was added, and the mixture was extracted with ethyl acetate. The ethyl acetate layer was washed with saturated brine, dried (MgSO₄), and concentrated. The residue was crystallized from ethyl acetate-hexane to give (2E)-3-[1-(2,4-dichlorobenzyl)-3-(2-methoxyethoxy)-1H-pyrazol-5-yl]acrylic acid (300 mg, yield: 91%) as colorless crystals. melting point 171-172° C.

### Reference Example 86

A mixture of methyl 3-hydroxy-1H-pyrazole-5-carboxylate (68.6 g), chloromethylmethyl ether (46.7 g), potassium carbonate (100 g) and N,N-dimethylformamide (350 ml) was stirred at room temperature for 15 hr, poured into water, and the mixture was extracted with ethyl acetate. The aqueous layer was extracted with chloroform, the extracts were combined, and the mixture was concentrated. The residue was dissolved in chloroform, and the solution was washed with water and saturated brine, dried (MgSO₄), and concentrated. The residue was subjected to silica gel column chromatography, eluted with ethyl acetate-hexane (1:12-1:2, v/v), concentrated, and crystallized from ethyl acetate-hexane to give methyl 3-(methoxymethoxy)-1H-pyrazole-5-carboxylate (23.4 g, yield: 26%) as colorless crystals. melting point 54-55° C.

### Reference Example 87

A mixture of methyl 3-(methoxymethoxy)-1H-pyrazole-5-carboxylate (23.4 g), 2-chloro-4-trifluoromethylbenzyl chloride (30.0 g), potassium carbonate (26.1 g) and N,N-dimethylformamide (120 ml) was stirred at room temperature for 15 hr, poured into water, and the mixture was extracted with ethyl acetate. The extract was washed with water and saturated brine, dried (MgSO₄), and concentrated. The residue was subjected to silica gel column chromatography, and eluted with ethyl acetate-hexane (1:24-1:10, v/v) to give methyl 1-[2-chloro-4-(trifluoromethyl)benzyl]-3-(methoxymethoxy)-1H-pyrazole-5-carboxylate (34.1 g, yield: 71%) as a colorless oil.

¹H-NMR (300 MHz, CDCl₃) δ:3.53 (3H, s), 3.83 (3H, s), 5.24 (2H, s), 5.79 (2H, s), 6.46 (1H, s), 6.70 (1H, d, J=8.1 Hz), 7.43 (1H, dd, J=0.9, 8.1 Hz), 7.65 (1H, d, J=0.9 Hz).

### Reference Example 88

A mixture of methyl 1-[2-chloro-4-(trifluoromethyl)benzyl]-3-(methoxymethoxy)-1H-pyrazole-5-carboxylate (34.1 g), a 1N aqueous sodium hydroxide solution (150 ml), tetrahydrofuran (250 ml) and ethanol (250 ml) was stirred at 50° C. for 1 hr. The reaction mixture was concentrated, 1N hydrochloric acid (200 ml) was added, and the mixture was extracted with ethyl acetate. The ethyl acetate layer was washed with saturated brine, dried (MgSO₄), and concentrated. The residue was crystallized from ethyl acetate-hexane to give 1-[2-chloro-4-(trifluoromethyl)benzyl]-3-(methoxymethoxy)-1H-pyrazole-5-carboxylic acid (28.1 g, yield: 86%) as colorless crystals. melting point 133-134° C.

### Reference Example 89

A mixture of N,O-dimethylhydroxylamine hydrochloride (9.01 g), triethylamine (9.35 g) and N,N-dimethylformamide (300 ml) was stirred at room temperature for 30 min, 1-[2-chloro-4-(trifluoromethyl)benzyl]-3-(methoxymethoxy)-1H-pyrazole-5-carboxylic acid (28.1 g), 1-ethyl-3-(3-dimethylaminopropyl)carbodiimide hydrochloride (17.7 g) and 1-hydroxybenzotriazole monohydrate (14.2 g) were added, and the mixture was stirred at room temperature for 15 hr. The reaction mixture was concentrated, and the residue was partitioned between water and ethyl acetate. The ethyl acetate layer was washed with 1N hydrochloric acid, saturated aqueous sodium hydrogencarbonate solution, water and saturated brine, dried (MgSO₄), and concentrated. The residue was subjected to silica gel column chromatography, and eluted with ethyl acetate-hexane (1:19-1:1, v/v) to give 1-[2-chloro-4-(trifluoromethyl)benzyl]-N-methoxy-3-(methoxymethoxy)-N-methyl-1H-pyrazole-5-carboxamide (29.1 g, yield: 93%) as a colorless oil.

¹H-NMR (300 MHz, CDCl₃) δ:3.29 (3H, s), 3.53 (3H, s), 3.67 (3H, s), 5.25 (2H, s), 5.70 (2H, s), 6.41 (1H, s), 6.75 (1H, d, J=8.1 Hz), 7.40 (1H, d, J=8.1 Hz), 7.62 (1H, s).

### Reference Example 90

To a solution of 1-[2-chloro-4-(trifluoromethyl)benzyl]-N-methoxy-3-(methoxymethoxy)-N-methyl-1H-pyrazole-5-carboxamide (29.1 g) in tetrahydrofuran (500 ml) was added a 1.5 M solution (71.0 ml) of diisobutylaluminum hydride in toluene at 0° C., and the mixture was stirred for 1 hr. Methanol was added to quench the reaction. The reaction mixture was poured into a 10% aqueous Rochelle salt solution, and the mixture was stirred and extracted with diethyl ether. The extract was washed with saturated brine, dried (MgSO₄), and concentrated. The residue was subjected to silica gel column chromatography, and eluted with ethyl acetate-hexane (1:19-1:2, v/v) to give 1-[2-chloro-4-(trifluoromethyl)benzyl]-3-(methoxymethoxy)-1H-pyrazole-5-carbaldehyde (21.2 g, yield: 85%) as a colorless oil.

¹H-NMR (300 MHz, CDCl₃) δ:3.53 (3H, s), 5.75 (2H, s), 6.51 (1H, s), 6.75 (1H, d, J=8.1 Hz), 7.40 (1H, d, J=8.1 Hz), 7.65 (1H, s); 9.76 (1H, s)

### Reference Example 91

Under ice-cooling, to a solution of ethyl diethylphosphonoactate (16.6 g) in N,N-dimethylformamide (100 ml) was added sodium hydride (60% in oil, 2.56 g), and the mixture was stirred at room temperature for 30 min. The reaction mixture was ice-cooled, a solution of 1-[2-chloro-4-(trifluoromethyl)benzyl]-3-(methoxymethoxy)-1H-pyrazole-5-carbaldehyde (17.2 g) in tetrahydrofuran (110 ml) was added, and the mixture was stirred for 1 hr. The mixture was warmed to room temperature and further stirred for 1 hr. A saturated aqueous ammonium chloride solution was added to quench the reaction. The reaction mixture was concentrated, and the residue was partitioned between water and ethyl acetate. The ethyl acetate layer was washed with water and saturated brine, dried (MgSO₄), and concentrated. The residue was subjected to silica gel column chromatography, and eluted with ethyl acetate-hexane (1:19-1:3, v/v) to give ethyl (2E)-3-{1-[2-chloro-4-(trifluoromethyl)benzyl]-3-(methoxymethoxy)-1H-pyrazol-5-yl}acrylate (12.4 g, yield: 60%) as a colorless oil.

¹H-NMR (300 MHz, CDCl₃) δ:1.30 (3H, t, J=7.2 Hz), 3.53 (3H, s), 4.23 (2H, t, J=7.2 Hz), 5.23 (2H, s), 5.43 (2H, s), 6.18 (1H, s), 6.32 (1H, d, J=15.5 Hz), 6.83 (1H, d, J=8.1 Hz), 7.39 (1H, d, J=15.5 Hz), 7.43 (1H, d, J=8.1 Hz), 7.66 (1H, s).

### Reference Example 92

Ethyl (2E)-3-{1-[2-Chloro-4-(trifluoromethyl)benzyl]-3-(methoxymethoxy)-1H-pyrazol-5-yl}acrylate (8.40 g), concentrated hydrochloric acid (0.30 ml) and methanol (70 ml) were stirred with heating under reflux for 5 hr. The reaction mixture was concentrated, and the residue was crystallized from diisopropyl ether-hexane to give ethyl (2E)-3-{1-[2-chloro-4-(trifluoromethyl)benzyl]-3-hydroxy-1H-pyrazol-5-yl}acrylate (6.53 g) as a white solid.

¹H-NMR (300 MHz, CDCl₃) δ:1.30 (3H, t, J=7.2 Hz), 4.23 (2H, q, J=7.2 Hz), 5.36 (2H, s), 5.98 (1H, s), 6.34 (1H, d, J=15.8 Hz), 6.84 (1H, d, J=8.1 Hz), 7.36 (1H, d, 8.1 Hz), 7.67 (1H, s).

### Reference Example 93

Ethyl (2E)-3-{1-[2-Chloro-4-(trifluoromethyl)benzyl]-3-hydroxy-1H-pyrazol-5-yl}acrylate (1.20 g), 2-iodopropane (653 mg), potassium carbonate (663 mg) and N,N-dimethylformamide (20 ml) were stirred at room temperature for 15 hr, the reaction mixture was poured into water, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:19-7:3, v/v) to give (2E)-,3-{1-[2-chloro-4-(trifluoromethyl)benzyl]-3-isopropoxy-1H-pyrazol-5-yl}ethyl acrylate (1.08 g, 81%) as white crystals.

A mixture of (2E)-3-{1-[2-chloro-4-(trifluoromethyl)benzyl]-3-isopropoxy-1H-pyrazol-5-yl}ethyl acrylate (1.38 g), a 1N aqueous sodium hydroxide solution (5.0 ml), tetrahydrofuran (10 ml) and ethanol (10 ml) was stirred at 50° C. for 1 hr. The reaction mixture was concentrated, 1N hydrochloric acid (50 ml) was added, and the mixture was extracted with ethyl acetate. The ethyl acetate layer was washed with saturated brine, dried (MgSO₄), and concentrated. The residue was crystallized from ethyl acetate-hexane to give (2E)-3-{1-[2-chloro-4-(trifluoromethyl)benzyl]-3-isopropoxy-1H-pyrazol-5-yl}acrylic acid (1.22 g, yield: 95%) as colorless crystals. melting point 170-171° C.

### Reference Example 94

To a solution of ethyl (2E)-3-{1-[2-chloro-4-(trifluoromethyl)benzyl]-3-hydroxy-1H-pyrazol-5-yl}acrylate (500 mg), (3-methyloxetan-3-yl)methanol (160 mg) and tributylphosphine (540 mg) in tetrahydrofuran (15 ml) was added 1,1′-(azodicarbonyl)dipiperidine (620 mg), and the mixture was stirred at room temperature for 2 hr and concentrated. Diisopropyl ether was added to the residue, the resulting insoluble material was filtered off, and the filtrate was concentrated. The residue was subjected to silica gel column chromatography, eluted with ethyl acetate-hexane (3:17-3:7, v/v), concentrated, and crystallized from ethyl acetate-hexane to give ethyl (2E)-3-{1-[2-chloro-4-(trifluoromethyl)benzyl]-3-[(3-methyloxetan-3-yl)methoxy]-1H-pyrazol-5-yl}acrylate (560 mg, yield: 91%) as a colorless oil.

A mixture of ethyl (2E)-3-{1-[2-chloro-4-(trifluoromethyl)benzyl]-3-[(3-methyloxetan-3-yl)methoxy]-1H-pyrazol-5-yl}acrylate (560 mg), a 1N aqueous sodium hydroxide solution (2.4 ml), tetrahydrofuran (7 ml) and ethanol (7 ml) was stirred at 50° C. for 1 hr. The reaction mixture was concentrated, 1N hydrochloric acid (20 ml) was added, and the mixture was extracted with ethyl acetate. The ethyl acetate layer was washed with saturated brine, dried (MgSO₄), and concentrated. The residue was crystallized from ethyl acetate-hexane to give (2E)-3-{1-[2-chloro-4-(trifluoromethyl)benzyl]-3-[(3-methyloxetan-3-yl)methoxy]-1H-pyrazol-5-yl}acrylic acid (425 mg, yield: 82%) as colorless crystals. melting point 148-149° C.

### Reference Example 95

To a solution (heated to 60° C.) of ethyl (2E)-3-[1-(2,4-dichlorobenzyl)-3-hydroxy-1H-pyrazol-5-yl]acrylate (1.50 g), tetrahydro-2H-pyran-4-ol (893 mg) and tributylphosphine (1.77 g) in tetrahydrofuran (60 ml) was added 1,1′-(azodicarbonyl)dipiperidine (1.77 g), and the mixture was stirred for 15 hr. After cooling to room temperature, the reaction mixture was concentrated. Diisopropyl ether was added to the residue, the resulting insoluble material was filtered off, and the filtrate was concentrated. The residue was subjected to silica gel column chromatography, eluted with ethyl acetate-hexane (1:19-1:3, v/v), concentrated, and crystallized from ethyl acetate-hexane to give ethyl (2E)-3-[1-(2,4-dichlorobenzyl)-3-(tetrahydro-2H-pyran-4-yloxy)-1H-pyrazol-5-yl]acrylate (1.47 g, yield: 79%) as colorless crystals.

A mixture of ethyl (2E)-3-[1-(2,4-dichlorobenzyl)-3-(tetrahydro-2H-pyran-4-yloxy)-1H-pyrazol-5-yl]acrylate (1.50 g), a 1N aqueous sodium hydroxide solution (8.0 ml), tetrahydrofuran (15 ml) and ethanol (15 ml) was stirred at 50° C. for 1 hr. The reaction mixture was concentrated, 1N hydrochloric acid (100 ml) was added, and the mixture was extracted with ethyl acetate. The ethyl acetate layer was washed with saturated brine, dried (MgSO₄), and concentrated. The residue was crystallized from ethyl acetate-hexane to give (2E)-3-[1-(2,4-dichlorobenzyl)-3-(tetrahydro-2H-pyran-4-yloxy)-1H-pyrazol-5-yl]acrylic acid (1.35 g, yield: 96%) as colorless crystals. melting point 191-192° C.

### Reference Example 96

To a solution (heated to 60° C.) of ethyl (2E)-3-{1-[2-chloro-4-(trifluoromethyl)benzyl]-3-hydroxy-1H-pyrazol-5-yl}acrylate (1.26 g), tetrahydro-2H-pyran-4-ol (687 mg) and tributylphosphine (1.36 g) in tetrahydrofuran (50 ml) was added 1,1′-(azodicarbonyl)dipiperidine (1.70 g), and the mixture was stirred for 15 hr. After cooling to room temperature, the reaction mixture was concentrated. Diisopropyl ether was added to the residue, the resulting insoluble material was filtered off, and the filtrate was concentrated. The residue was subjected to silica gel column chromatography, eluted with ethyl acetate-hexane (1:19-1:3, v/v) to give ethyl (2E)-3-[1-[2-chloro-4-(trifluoromethyl)benzyl]-3-(tetrahydro-2H-pyran-4-yloxy)-1H-pyrazol-5-yl]acrylate (1.16 g, yield: 75%) as colorless crystals.

A mixture of ethyl (2E)-3-[1-[2-chloro-4-(trifluoromethyl)benzyl]-3-(tetrahydro-2H-pyran-4-yloxy)-1H-pyrazol-5-yl]acrylate (1.16 g), a 1N aqueous sodium hydroxide solution (5.0 ml), tetrahydrofuran (10 ml) and ethanol (10 ml) was stirred at 50° C. for 1 hr. The reaction mixture was concentrated, 1N hydrochloric acid (100 ml) was added, and the mixture was extracted with ethyl acetate. The ethyl acetate layer was washed with saturated brine, dried (MgSO₄) and concentrated to give (2E)-3-[1-[2-chloro-4-(trifluoromethyl)benzyl]-3-(tetrahydro-2H-pyran-4-yloxy)-1H-pyrazol-5-yl]acrylic acid (1.07 g, yield: 98%) as a white solid.

¹H-NMR (300 MHz, CDCl₃) δ:1.73-1.87 (2H, m), 2.00-2.13 (2H, m), 3.48-3.63 (2H, m), 3.92-4.04 (2H, m), 4.64-4.76 (1H, m), 5.41 (2H, s), 6.09 (1H, s), 6.29 (1H, d, J=15.6 Hz), 6.77 (1H, d, J=8.1 Hz), 7.44 (1H, d, J=8.1 Hz), 7.45 (1H, d, J=15.6 Hz), 7.67 (1H, s).

### Reference Example 97

To a mixture of potassium tert-butoxide (94.1 g) and tetrahydrofuran (500 ml) was added dropwise a mixture of acetylcyclopropane (50.0 g) and diethyl oxalate (87.8 g), and the mixture was stirred at room temperature for 15 hr. Acetic acid (81.3 ml) and hydrazine monohydrate (32.7 g) were added to the reaction mixture, and the mixture was stirred with heating under reflux for 2 hr. After cooling to room temperature, the reaction mixture was concentrated, and the residue was partitioned between water and ethyl acetate. The ethyl acetate layer was washed with saturated aqueous sodium carbonate solution and saturated brine, dried (MgSO₄), and concentrated. The residue was crystallized from diisopropyl ether to give ethyl 3-cyclopropyl-1H-pyrazole-5-carboxylate (64.5 g, yield: 60%) as brown crystals.

¹H-NMR (300 MHz, CDCl₃) δ:0.68-0.82 (2H, m), 0.90-1.06 (2H, m), 1.34 (3H, t, J=7.2 Hz), 1.88-2.04 (1H, m), 4.36 (2H, q, J=7.2 Hz), 6.45 (1H, s), 11.30 (1H, brs).

### Reference Example 98

A mixture of ethyl 3-cyclopropyl-1H-pyrazole-5-carboxylate (20.0 g), 2-chloro-4-trifluoromethylbenzyl chloride (28.0 g), potassium carbonate (23.0 g) and N,N-dimethylformamide (200 ml) was stirred at room temperature for 15 hr, concentrated, and the residue was partitioned between water and ethyl acetate. The ethyl acetate layer was washed with water and saturated brine, dried (MgSO₄), and concentrated. The residue was subjected to silica gel column chromatography, and eluted with ethyl acetate-hexane (1:19-3:7, v/v) to give ethyl 1-[2-chloro-4-(trifluoromethyl)benzyl]-3-cyclopropyl-1H-pyrazole-5-carboxylate (23.2 g, yield: 56%) as a pale-yellow oil.

¹H-NMR (300 MHz, CDCl₃) δ:0.72-0.80 (2H, m), 0.92-1.02 (2H, m), 1.29 (3H, t, J=7.2 Hz), 1.90-2.02 (1H, m), 4.25 (2H, q, J=7.2 Hz), 5.82 (2H, s), 6.53 (1H, d, J=8.1 Hz), 6.62 (1H, s), 7.38 (1H, d, J=8.1 Hz), 7.64 (1H, s)

### Reference Example 99

To a solution of ethyl 1-[2-chloro-4-(trifluoromethyl)benzyl]-3-cyclopropyl-1H-pyrazole-5-carboxylate (23.2 g) in tetrahydrofuran (300 ml) was added a 1.5 M solution (104 ml) of diisobutylaluminum hydride in toluene at 0° C. The mixture was stirred at room temperature for 1 hr, and sodium sulfate 10 hydrate was added to quench the reaction. The insoluble material was filtered off, and the filtrate was concentrated. The residue was dissolved in ethyl acetate, dried (MgSO₄), and concentrated. The residue was crystallized from ethyl acetate-hexane to give {1-[2-chloro-4-(trifluoromethyl)benzyl]-3-cyclopropyl-1H-pyrazol-5-yl}methanol (17.2 g, yield: 84%) as colorless crystals.

¹H-NMR (300 MHz, CDCl₃) δ:0.68-0.75 (2H, m), 0.88-0.98 (2H, m), 1.63 (1H, t, J=5.7 Hz), 1.88-1.99 (1H, m), 4.55 (2H, d, J=5.7 Hz), 5.45 (2H, s), 5.96 (1H, s), 6.66 (1H, d, J=8.1 Hz), 7.41 (1H, d, J=8.1 Hz), 7.63 (1H, s).

### Reference Example 100

Under a nitrogen atmosphere, to a solution of oxalyl chloride (13.1 g) in dichloromethane (150 ml) was added dimethylsulfoxide (12.1 g) at −78° C., and the mixture was stirred for 5 min. A solution of {1-[2-chloro-4-(trifluoromethyl)benzyl]-3-cyclopropyl-1H-pyrazol-5-yl}methanol (17.1 g) in dichloromethane (200 ml) was added. After stirring at −78° C. for 1 hr, triethylamine (26.2 g) was added, and the reaction mixture was warmed to room temperature and further stirred for 1 hr. The reaction mixture was concentrated, and the residue was partitioned between water and ethyl acetate. The ethyl acetate layer was washed with 1N hydrochloric acid and saturated brine, dried (MgSO₄), and concentrated. The residue was crystallized from ethyl acetate-hexane to give 1-[2-chloro-4-(trifluoromethyl)benzyl]-3-cyclopropyl-1H-pyrazole-5-carbaldehyde (14.3 g, yield: 84%) as pale-yellow crystals. melting point 94-96° C.

### Reference Example 101

Under ice-cooling, to a solution of ethyl diethylphosphonoacetate (14.6 g) in N,N-dimethylformamide (100 ml) was added sodium hydride (60% in oil, 2.26 g), and the mixture was stirred at room temperature for 30 min. The reaction mixture was ice-cooled, a solution of 1-[2-chloro-4-(trifluoromethyl)benzyl]-3-cyclopropyl-1H-pyrazole-5-carbaldehyde (14.3 g) in tetrahydrofuran (100 ml) was added, and the mixture was stirred for 1 hr. The reaction mixture was warmed to room temperature and further stirred for 1 hr. A saturated aqueous ammonium chloride solution was added to quench the reaction. The reaction mixture was concentrated, and the residue was partitioned between water and ethyl acetate. The ethyl acetate layer was washed with water and saturated brine, dried (MgSO₄), and concentrated. The residue was subjected to silica gel column chromatography, and eluted with ethyl acetate-hexane (1:19-1:3, v/v) to give ethyl (2E)-3-{1-[2-chloro-4-(trifluoromethyl)benzyl]-3-cyclopropyl-1H-pyrazol-5-yl}acrylate (16.9 g, yield: 97%) as a colorless oil.

¹H-NMR (300 MHz, CDCl₃) δ:0.72-0.80 (2H, m), 0.92-1.01 (2H, m), 1.30 (3H, t, J=7.2 Hz), 1.88-2.00 (1H, m), 4.22 (2H, q, J=7.2 Hz), 5.49 (2H, s), 6.29 (1H, d, J=15.8 Hz), 6.66 (1H, d, J=8.1 Hz), 7.38 (1H, d, J=15.8 Hz), 7.42 (1H, d, J=8.1 Hz), 7.66 (1H, s)

### Reference Example 102

A mixture of ethyl (2E)-3-{1-[2-chloro-4-(trifluoromethyl)benzyl]-3-cyclopropyl-1H-pyrazol-5-yl}acrylate (8.90 g), 5% palladium-carbon (1.91 g) and tetrahydrofuran (15 ml) was hydrogenated at room temperature under atmospheric pressure. The reaction mixture was filtrated, and the filtrate was concentrated. The residue was dissolved in tetrahydrofuran (80 ml), ethanol (80 ml) and 1N aqueous sodium hydroxide solution (40 ml) were added, and the mixture was stirred at 50° C. for 1 hr. The reaction mixture was concentrated, 1N hydrochloric acid (200 ml) was added, and the mixture was extracted with ethyl acetate. The ethyl acetate layer was washed with saturated brine, dried (MgSO₄), and concentrated. The residue was crystallized from ethyl acetate-diisopropyl ether to give 3-{1-[2-chloro-4-(trifluoromethyl)benzyl]-3-cyclopropyl-1H-pyrazol-5-yl}propanoic acid (1.80 g, yield: 86%, containing 3-{1-[2-chloro-4-(trifluoromethyl)benzyl]-3-propyl-1H-pyrazol-5-yl}propanoic acid by 9%) as a white solid.

¹H-NMR (300 MHz, CDCl₃) δ:0.66-0.74 (2H, m), 0.87-0.97 (2H, m), 1.85-1.98 (1H, m), 2.60-2.67 (2H, m), 2.72-2.79 (2H, m), 5.36 (2H, s), 5.80 (1H, s), 6.59 (1H, d, J=8.1 Hz), 7.40 (1H, dd, J=0.9, 8.1 Hz), 7.66 (1H, d, J=0.9 Hz).

### Reference Example 103

A mixture of ethyl (2E)-3-{1-[2-chloro-4-(trifluoromethyl)benzyl]-3-cyclopropyl-1H-pyrazol-5-yl}acrylate (8.00 g), a 1N aqueous sodium hydroxide solution (40 ml), tetrahydrofuran (80 ml) and ethanol (80 ml) was stirred at 50° C. for 1 hr. The reaction mixture was concentrated, 1N hydrochloric acid (200 ml) was added, and the mixture was extracted with ethyl acetate. The ethyl acetate layer was washed with saturated brine, dried (MgSO₄) and concentrated to give (2E)-3-{1-[2-chloro-4-(trifluoromethyl)benzyl]-3-cyclopropyl-1H-pyrazol-5-yl}acrylic acid. (6.40 g, yield: 86%) as a white solid melting point 191-192° C.

### Reference Example 104

A mixture of ethyl (2E)-3-[1-[2-chloro-4-(trifluoromethyl)benzyl]-3-(cyclopropylmethoxy)-1H-pyrazol-5-yl]acrylate (690 mg), 5% palladium-carbon (130 mg) and tetrahydrofuran (15 ml) was hydrogenated at room temperature under atmospheric pressure. The reaction mixture was filtrated, and the filtrate was concentrated to give ethyl 3-[1-[2-chloro-4-(trifluoromethyl)benzyl]-3-(cyclopropylmethoxy)-1H-pyrazol-5-yl]propanoate (650 mg, yield: 94%) as a pale-yellow oil.

¹H-NMR (300 MHz, CDCl₃) δ:0.28-0.37 (2H, m), 0.54-0.64 (2H, m), 1.18-1.32 (4H, m), 2.53-2.64 (2H, m), 4.12 (2H, q, J=7.2 Hz), 5.26 (2H, s), 5.59 (1H, s), 6.68 (1H, d, J=8.1 Hz), 7.42 (1H, d, J=8.1 Hz), 7.64 (1H, s).

### Reference Example 105

A mixture of ethyl 3-[1-[2-chloro-4-(trifluoromethyl)benzyl]-3-(cyclopropylmethoxy)-1H-pyrazol-5-yl]propanoate (650 mg), a 1N aqueous sodium hydroxide solution (4.0 ml), tetrahydrofuran (8.0 ml) and ethanol (8.0 ml) was stirred at 50° C. for 1 hr. The reaction mixture was concentrated, 1N hydrochloric acid (50 ml) was added, and the mixture was extracted with ethyl acetate. The ethyl acetate layer was washed with saturated brine, dried (MgSO₄), and concentrated. The residue was crystallized from ethyl acetate-hexane to give 3-[1-[2-chloro-4-(trifluoromethyl)benzyl]-3-(cyclopropylmethoxy)-1H-pyrazol-5-yl]propanoic acid (510 mg, yield: 84%) as colorless crystals. melting point 115-116° C.

### Reference Example 106

To a mixture of potassium tert-butoxide (64.8 g) and tetrahydrofuran (500 ml) was added dropwise a mixture of 3,3-dimethyl-2-butanone (41.0 g) and diethyl oxalate (60.4 g), and the mixture was stirred at room temperature for 15 hr. Acetic acid (56.0 ml) and hydrazine monohydrate (22.5 g) were added to the reaction mixture, and the mixture was stirred with heating under reflux for 2 hr. After cooling to room temperature, the reaction mixture was concentrated, and the residue was poured into water. The resulting precipitate was collected by filtration to give ethyl 3-tert-butyl-1H-pyrazole-5-carboxylate (65.6 g, yield: 82%) as an orange solid.

¹H-NMR (300 MHz, CDCl₃) δ:1.278 (9H, s), 1.282 (3H, t, J=7.2 Hz), 4.25 (2H, q, J=7.2 Hz), 6.49 (1H, brs), 13.22 (1H, brs).

### Reference Example 107

A mixture of ethyl 3-tert-butyl-1H-pyrazole-5-carboxylate (20.0 g), 2-chloro-4-trifluoromethylbenzyl chloride (24.1 g), potassium carbonate (21.1 g) and N,N-dimethylformamide (200 ml) was stirred at room temperature for 15 hr, and the reaction mixture was concentrated. The residue was partitioned between water and ethyl acetate. The ethyl acetate layer was washed with water and saturated brine, dried (MgSO₄), and concentrated. The residue was subjected to silica gel column chromatography, and eluted with ethyl acetate-hexane (1:19-1:6, v/v) to give ethyl 3-tert-butyl-1-[2-chloro-4-(trifluoromethyl)benzyl]-1H-pyrazole-5-carboxylate (36.8 g, yield: 93%) as a pale-yellow oil.

¹H-NMR (300 MHz, CDCl₃) δ:1.29 (3H, t, J=7.2 Hz), 1.34 (9H, s), 4.25 (2H, q, J=7.2 Hz), 5.85 (2H, s), 6.43 (1H, d, J=8.1 Hz), 6.83 (1H, s), 7.37 (1H, d, J=8.1 Hz), 7.64 (1H, s).

### Reference Example 108

To a solution of ethyl-3-tert-butyl-1-[2-chloro-4-(trifluoromethyl)benzyl]-1H-pyrazole-5-carboxylate (23.2 g) in tetrahydrofuran (400 ml) was added a 1.5 M solution (158 ml) of diisobutylaluminum hydride in toluene at 0° C., and the mixture was stirred at room temperature for 1 hr. Sodium sulfate 10 hydrate was added to quench the reaction. The insoluble material was filtered off, and the filtrate was dried (MgSO₄) and concentrated. The residue was crystallized from ethyl acetate-hexane to give {3-tert-butyl-1-[2-chloro-4-(trifluoromethyl)benzyl]-1H-pyrazol-5-yl}methanol (7.69 g, yield: 23%) as colorless crystals. melting point 115-117° C.

### Reference Example 109

Under a nitrogen atmosphere, to a solution of oxalyl chloride (7.47 g) in dichloromethane (50 ml) was added dimethylsulfoxide (6.89 g) at −78° C., and the mixture was stirred for 5 min. A solution of {3-tert-butyl-1-[2-chloro-4-(trifluoromethyl)benzyl]-1H-pyrazol-5-yl}methanol (10.2 g) in dichloromethane (80 ml) was added. After stirring at −78° C. for 1 hr, triethylamine (14.9 g) was added, and the reaction mixture was warmed to room temperature and further stirred for 1 hr. The reaction mixture was concentrated, and the residue was partitioned between water and ethyl acetate. The ethyl acetate layer was washed with 1N hydrochloric acid and saturated brine, dried (MgSO₄), and concentrated. The residue was crystallized from ethyl acetate-hexane to give 3-tert-butyl-1-[2-chloro-4-(trifluoromethyl)benzyl]-1H-pyrazole-5-carbaldehyde (8.16 g, yield: 81%) as pale-yellow crystals melting point 147-149° C.

### Reference Example 110

Under ice-cooling, to a solution of ethyl diethylphosphonoacetate (7.50 g) in N,N-dimethylformamide (50 ml) was added sodium hydride (60% in oil, 1.16 g), and the mixture was stirred at room temperature for 30 min. The reaction mixture was ice-cooled, a solution of 3-tert-butyl-1-[2-chloro-4-(trifluoromethyl)benzyl]-1H-pyrazole-5-carbaldehyde (8.16 g) in tetrahydrofuran (50 ml) was added, and the mixture was stirred for 1 hr. The reaction mixture was warmed to room temperature and further stirred for 1 hr. A saturated aqueous ammonium chloride solution was added to quench the reaction. The reaction mixture was concentrated, and the residue was partitioned between water and ethyl acetate. The ethyl acetate layer was washed with water and saturated brine, dried (MgSO₄), and concentrated. The residue was subjected to silica gel column chromatography, and eluted with ethyl acetate-hexane (1:19-1:4, v/v) to give ethyl (2E)-3-{3-tert-butyl-1-[2-chloro-4-(trifluoromethyl)benzyl]-1H-pyrazol-5-yl}acrylate (9.82 g, yield: 100%) as a colorless oil.

¹H-NMR (300 MHz, CDCl₃) δ:1.29 (3H, t, J=7.2 Hz), 1.33 (9H, s), 4.21 (2H, q, J=7.2 Hz), 5.53 (2H, s), 6.32 (1H, d, J=15.8 Hz), 6.56 (1H, d, J=8.4 Hz), 7.37 (1H, d, J=15.8 Hz), 7.41 (1H, d, J=8.4 Hz), 7.66 (1H, s).

### Reference Example 111

A mixture of ethyl (2E)-3-{3-tert-butyl-1-[2-chloro-4-(trifluoromethyl)benzyl]-1H-pyrazol-5-yl}acrylate (3.00 g), a 1N aqueous sodium hydroxide solution (20 ml), tetrahydrofuran (40 ml) and ethanol (40 ml) was stirred at 50° C. for 1 hr. The reaction mixture was concentrated, 1N hydrochloric acid (200 ml) was added, and the mixture was extracted with ethyl acetate. The ethyl acetate layer was washed with saturated brine, dried (MgSO₄), and concentrated. The residue was crystallized from ethyl acetate-hexane to give (2E)-3-{3-tert-butyl-1-[2-chloro-4-(trifluoromethyl)benzyl]-1H-pyrazol-5-yl}acrylic acid (2.24 g, yield: 80%) as colorless crystals melting point 175-176° C.

### Reference Example 112

A mixture of ethyl (2E)-3-{3-tert-butyl-1-[2-chloro-4-(trifluoromethyl)benzyl]-1H-pyrazol-5-yl}acrylate (3.00 g), 5% palladium-carbon (500 mg) and tetrahydrofuran (50 ml) was hydrogenated at room temperature under atmospheric pressure. The reaction mixture was filtrated, and the filtrate was concentrated to give ethyl 3-{3-tert-butyl-1-[2-chloro-4-(trifluoromethyl)benzyl]-1H-pyrazol-5-yl}propanoate (2.53 g, yield: 84%) as a colorless oil.

¹H-NMR (300 MHz, CDCl₃) δ:1.23 (3H, t, J=7.2 Hz), 1.31 (9H, s), 2.55-2.65 (2H, m), 2.72-2.82 (2H, m), 4.12 (2H, q, J=7.2 Hz), 5.39 (2H, s), 6.00 (1H, s), 6.47 (1H, d, J=8.1 Hz), 7.41 (1H, d, J=8.1 Hz), 7.63 (1H, s).

### Reference Example 113

A mixture of ethyl 3-{3-tert-butyl-1-[2-chloro-4-(trifluoromethyl)benzyl]-1H-pyrazol-5-yl}propanoate (2.53 g), a 1N aqueous sodium hydroxide solution (15 ml), tetrahydrofuran (30 ml) and ethanol (30 ml) was stirred at 50° C. for 1 hr. The reaction mixture was concentrated, 1N hydrochloric acid (200 ml) was added, and the mixture was extracted with ethyl acetate. The ethyl acetate layer was washed with saturated brine, dried (MgSO₄), and concentrated. The residue was crystallized from ethyl acetate-hexane to give 3-{3-tert-butyl-1-[2-chloro-4-(trifluoromethyl)benzyl]-1H-pyrazol-5-yl}propanoic acid (2.08 g, yield: 88%) as colorless crystals. melting point 128-129° C.

### Reference Example 114

A mixture of tert-butyl ({(E)-2-[1-(2,4-dichlorobenzyl)-3-isopropoxy-1H-pyrazol-5-yl]vinyl}sulfonyl)carbamate (1.03 g) and trifluoroacetic acid (20 ml) was stirred at room temperature for 1 hr and the reaction mixture was concentrated. A saturated aqueous sodium hydrogencarbonate solution was added to the residue, and the mixture was extracted with ethyl acetate. The extract was washed with saturated brine, dried (MgSO₄), and concentrated. The residue was crystallized from ethyl acetate-hexane to give (E)-2-[1-(2,4-dichlorobenzyl)-3-isopropoxy-1H-pyrazol-5-yl]ethylenesulfonamide (710 mg, yield: 87%) as colorless crystals. melting point 158-160° C.

### Reference Example 115

A mixture of (E)-2-[1-(2,4-dichlorobenzyl)-3-isopropoxy-1H-pyrazol-5-yl]ethylenesulfonamide (350 mg), 5% palladium carbon (140 mg) and tetrahydrofuran (15 ml) was hydrogenated at room temperature under atmospheric pressure for 15 hr. The reaction mixture was filtrated, and the filtrate was concentrated. A mixture of the residue, 5% palladium-carbon (200 mg) and tetrahydrofuran (15 ml) was hydrogenated again at room temperature under atmospheric pressure for 15 hr. The reaction mixture was filtrated, and the filtrate was concentrated. The residue was crystallized from ethyl acetate-hexane to give 2-[1-(2,4-dichlorobenzyl)-3-isopropoxy-1H-pyrazol-5-yl]ethanesulfonamide (260 mg, yield: 74%) as colorless crystals. melting point 142-143° C.

### Reference Example 116

A mixture of tert-butyl ({(E)-2-[3-butoxy-1-(2,4-dichlorobenzyl)-1H-pyrazol-5-yl]vinyl}sulfonyl)carbamate (1.55 g) and trifluoroacetic acid (15 ml) was stirred at room temperature for 1 hr, and the reaction mixture was concentrated. A saturated aqueous sodium hydrogencarbonate solution was added to the residue, and the mixture was extracted with ethyl acetate. The extract was washed with saturated brine, dried (MgSO₄), and concentrated. The residue was crystallized from ethyl acetate-hexane to give (E)-2-[1-(2,4-dichlorobenzyl)-3-butoxy-1H-pyrazol-5-yl]ethylenesulfonamide (1.10 g, yield: 89%) as colorless crystals. melting point 105-108° C.

### Reference Example 117

A mixture of (E)-2-[1-(2,4-dichlorobenzyl)-3-butoxy-1H-pyrazol-5-yl]ethylenesulfonamide (1.35 g), 5% palladium-carbon (920 mg) and tetrahydrofuran (50 ml) was hydrogenated at room temperature under atmospheric pressure for 24 hr. The reaction mixture was filtrated, and the filtrate was concentrated. The residue was crystallized from ethyl acetate-hexane to give 2-[3-butoxy-1-(2,4-dichlorobenzyl))-1H-pyrazol-5-yl]ethanesulfonamide (1.10 g, yield: 81%) as colorless crystals. melting point 134-136° C.

### Reference Example 118

To a solution of ethyl 3-{1-[2-chloro-4-(trifluoromethyl)benzyl]-3-isopropoxy-1H-pyrazol-5-yl}propionate (5.70 g) in tetrahydrofuran (15 ml) and ethanol (15 ml) was added a 1N aqueous sodium hydroxide solution (29.0 ml), and the mixture was stirred at 50° C. for 30 min. 1N Hydrochloric acid (29.0 ml) was added to the reaction mixture, and the mixture was extracted with ethyl acetate. The organic layer was washed with saturated brine, dried (MgSO₄), filtrated and concentrated to give a white solid. Recrystallization from ethyl acetate-hexane gave 3-{1-[2-chloro-4-(trifluoromethyl)benzyl]-3-isopropoxy-1H-pyrazol-5-yl}propionic acid (4.09 g, yield: 77%) as white fine needles melting point 121.5-122.0° C.

### Reference Example 119

To a solution of methyl 3-butoxy-1-(2,4-dichlorobenzyl)-1H-pyrazole-5-carboxylate (41.63 g) in tetrahydrofuran (200 ml) was added a 1.5 M solution (300 ml), of diisobutylaluminum hydride in toluene under ice-cooling, and the mixture was stirred for 10 min. A saturated aqueous ammonium chloride solution (85.0 ml) was added to the reaction mixture, and the precipitated solid was filtered off and the filtrate was concentrated. The obtained residue was subjected to silica gel column chromatography, and eluted with ethyl acetate-hexane (1:9-1:2, v/v) to give [3-butoxy-1-(2,4-dichlorobenzyl)-1H-pyrazol-5-yl]methanol (24.51 g, yield: 64%) as a white solid.

¹H-NMR (300 MHz, CDCl₃) δ: 0.95 (3H, t, J=7.3 Hz), 1.20-1.53 (2H, m), 1.66-1.89 (2H, m), 4.05-4.21 (2H, m), 4.47-4.63 (2H, m), 5.28 (2H, d, J=1.5 Hz), 5.72 (1H, d, J=2.1 Hz), 6.59-6.68 (1H, m), 7.14 (1H, d, J=8.5 Hz), 7.34-7.41 (1H, m)

### Reference Example 120

To a solution of [3-butoxy-17(2,4-dichlorobenzyl)-1H-pyrazol-5-yl]methanol (1.71 g) in tetrahydrofuran (40 ml) were added tributylphosphine (2.40 ml), acetone cyanhydrin (0.70 ml) and 1,1′-azodicarbonyldipiperidine (2.40 g), and the mixture was stirred at room temperature for 30 min. The reaction mixture was concentrated, the obtained solid was washed with diisopropyl ether, and the filtrate was concentrated. The obtained residue was subjected to silica gel column chromatography, and eluted with ethyl acetate-hexane (hexane alone to 1:3, v/v) to give a yellow oil.

To a solution of the obtained oil in ethanol (25 ml) was added 4N aqueous sodium hydroxide solution (13.0 ml), and the mixture was stirred with heating under reflux for 1.5 hr. After cooling to room temperature, 6N hydrochloric acid (9.0 ml) was added to the reaction mixture, and the mixture was diluted with toluene, concentrated, and dissolved in ethyl acetate. The organic layer was washed with saturated brine, dried (MgSO₄), filtrated and concentrated to give a white solid. Recrystallization from ethyl acetate-hexane gave [3-butoxy-1-(2,4-dichlorobenzyl)-1H-pyrazol-5-yl]acetic acid (890 mg, yield: 48%) as white crystals. melting point 105-108° C.

### Reference Example 121

To a solution of ethyl[3-butoxy-1-(2,4-dichlorobenzyl)-1H-pyrazol-5-yl]acrylate (6.31 g) in tetrahydrofuran (100 ml) was added 5% palladium-carbon (4.30 g), and the mixture was stirred under a hydrogen atmosphere at room temperature for 3 hr. The reaction mixture was filtrated and the filtrate was concentrated to give pale yellow oil.

To a solution of the obtained oil in tetrahydrofuran (100 ml) was added a 1.5 M solution (40 ml) of diisobutylaluminum hydride in toluene under ice-cooling, and the mixture was stirred for 10 min. A saturated aqueous ammonium chloride solution (12.0 ml) was added to the reaction mixture, and the precipitated solid was filtered off and the filtrate was concentrated. The obtained residue was subjected to silica gel column chromatography, and eluted With ethyl acetate-hexane (1:9-1:4, v/v) to give a pale-yellow oil.

To a solution of the obtained oil in tetrahydrofuran (100 ml) were added tributylphosphine (5.90 ml), acetone cyanhydrin (2.0 ml) and 1,1′-azodicarbonyldipiperidine (5.31 g), and the mixture was stirred at room temperature for 1 hr, and at 50° C. for 30 min. The reaction mixture was concentrated, the obtained solid was washed with diisopropyl ether, and the filtrate was concentrated. The obtained residue was subjected to silica gel column chromatography, and eluted with ethyl acetate-hexane (hexane alone to 1:4, v/v) to give a pale-yellow oil.

To a solution of the obtained oil in ethanol (30 ml) was added 4N aqueous sodium hydroxide solution (5.0 ml), and the mixture was stirred with heating under reflux for 1 hr and 20 min. Then a 4N aqueous sodium hydroxide solution (2.5 ml) was added, and the mixture was stirred overnight with heating under reflux. After cooling to room temperature, 6N hydrochloric acid (5.0 ml) was added to the reaction mixture, and the mixture was diluted with toluene, concentrated, and dissolved in ethyl acetate. The organic layer was washed with saturated brine, dried (MgSO₄), filtrated and concentrated to give 4-[3-butoxy-1-(2,4-dichlorobenzyl)-1H-pyrazol-5-yl]butanoic acid (2.86 g, yield: 47%) as an orange oil. A part thereof was subjected to silica gel column chromatography, and eluted with ethyl acetate-hexane (15:85-3:2, v/v) to give a white solid. Recrystallization from ethyl acetate-hexane gave white fine needles melting point 60.5-61.0° C.

### Reference Example 122

4-[3-Butoxy-1-(2,4-dichlorobenzyl)-1H-pyrazol-5-yl]butanoic acid (630 mg) was added to a borane-tetrahydrofuran complex 1.0 M tetrahydrofuran solution (6.0 ml), and the mixture was stirred at room temperature for 30 min. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:3, v/v) to give a colorless oil.

To a solution of the obtained oil in tetrahydrofuran (20 ml) were added tributylphosphine (0.80 ml), acetone cyanhydrin (0.25 ml) and 1,1′-azodicarbonyldipiperidine (0.80 g), and the mixture was stirred at room temperature for 1 hr. The reaction mixture was concentrated, the obtained solid was washed with diisopropyl ether, and the filtrate was concentrated. The obtained residue was subjected to silica gel column chromatography, and eluted with ethyl acetate-hexane (hexane alone to 3:7, v/v) to give a colorless oil.

To a solution of the obtained oil in ethanol (5 ml) was added a 4N aqueous sodium hydroxide solution (4.0 ml), and the mixture was stirred with heating under reflux for 3 hr. Then, a 8N aqueous sodium hydroxide solution (3.0 ml) was added, and the mixture was further stirred with heating under reflux for 4 hr. After cooling to room temperature, 1N hydrochloric acid was added to the reaction mixture, and the mixture was diluted with toluene, concentrated, and dissolved in ethyl acetate. The organic layer was washed with saturated brine, dried (MgSO₄), filtrated and concentrated to give 5-[3-butoxy-1-(2,4-dichlorobenzyl)-1H-pyrazol-5-yl]pentanoic acid (433 mg, yield: 63%) as a colorless oil.

¹H-NMR (300 MHz, CDCl₃) δ:0.95 (3H, t, J=7.4 Hz), 1.35-1.85 (8H, m), 2.32 (2H, t, J=6.9 Hz), 2.44 (2H, t, J=7.1 Hz), 4.10 (2H, q, J=6.7 Hz), 5.15 (2H, s), 5.55 (1H, s), 6.56 (1H, d, J=8.5 Hz), 7.14 (1H, dd, J=8.5, 2.1 Hz), 7.37 (1H, d, J=2.1 Hz)

### Reference Example 123

To a mixture of ethyl (2E)-3-{1-[2-chloro-4-(trifluoromethyl)benzyl]-3-hydroxy-1H-pyrazol-5-yl}acrylate (1.49 g), 2-methoxyethanol (0.347 ml), tributylphosphine (2.49 ml) and tetrahydrofuran (30 ml) was added 1,1′-azodicarbonyldipiperidine (2.02 g) at 50° C., and the mixture was stirred for 1 hr. The reaction solution was concentrated, diisopropyl ether was added to the residue, and the insoluble material was filtered off. The mother liquor was concentrated, and the obtained residue was subjected to silica gel column chromatography and eluted with ethyl acetate-hexane (1:100-1:4, v/v) to give a colorless oil (1.31 g).

A mixture of the obtained oil, a 1N aqueous sodium hydroxide solution (20 ml), tetrahydrofuran (20 ml) and ethanol (20 ml) was stirred at room temperature for 8 hr, 1N hydrochloric acid (20 ml) was added, and the mixture was extracted with ethyl acetate. The ethyl acetate layer was washed with saturated brine, dried (MgSO₄), and concentrated. The obtained crude crystals were recrystallized from tetrahydrofuran-hexane to give (2E)-3-[1-[2-chloro-4-(trifluoromethyl)benzyl]-3-(2-methoxyethoxy)-1H-pyrazol-5-yl]acrylic acid (1.14 g, yield: 70%) as colorless crystals.

¹H-NMR (300 MHz, CDCl₃) δ:3.44 (3H, s), 3.66-3.81 (2H, m), 4.22-4.40 (2H, m), 5.41 (2H, s), 6.13 (1H, s), 6.29 (1H, d, J=15.8 Hz), 6.77 (1H, d, J=8.1 Hz), 7.36-7.52 (2H, m), 7.66 (1H, d, J=0.9 Hz).

### Reference Example 124

Under ice-cooling, to a mixture of methyl 3-butoxypyrazole-5-carboxylate (43.1 g), potassium carbonate (36.0 g) and N,N-dimethylformamide (300 ml) was added 2,4-dichlorobenzyl chloride (33.2 ml), and the mixture was stirred at room temperature for 12 hr. The reaction mixture was concentrated, and the residue was partitioned between water and ethyl acetate. The ethyl acetate layer was washed with water and saturated brine, dried (MgSO₄), and concentrated. The residue was subjected to silica gel column chromatography, and eluted with ethyl acetate-hexane (1:20-1:15, v/v) to give methyl 3-butoxy-1-(2,4-dichlorobenzyl)-1H-pyrazole-5-carboxylate (56.0 g, yield: 72%, containing isomer by about 10%) as a colorless oil.

¹H-NMR (300 MHz, CDCl₃) δ:0.96 (3H, t, J=7.2 Hz), 1.40-1.53 (2H, m), 1.69-1.79 (2H, m), 3.81 (3H, s), 4.12 (2H, t, J=6.6 Hz), 5.67 (2H, s), 6.29 (1H, s), 6.53 (1H, d, J=8.7 Hz), 7.11 (1H, dd, J=2.1, 8.1 Hz), 7.38 (1H, d, J=2.1 Hz).

### Reference Example 125

To a mixture of methyl 3-butoxy-1-(2,4-dichlorobenzyl)-1H-pyrazole-5-carboxylate (55.6 g), tetrahydrofuran (200 ml) and methanol (100 ml) was added a solution of sodium hydroxide (12.5 g) in water (100 ml), and the mixture was stirred at room temperature for 2 hr. The reaction mixture was concentrated, 1N hydrochloric acid (320 ml) was added, and the mixture was extracted with ethyl acetate. The ethyl acetate layer was washed with saturated brine, dried (MgSO₄), and concentrated. The residue was crystallized from ethyl acetate-hexane to give 3-butoxy-1-(2,4-dichlorobenzyl)-1H-pyrazole-5-carboxylic acid (33.6 g, yield: 62%) as colorless crystals melting point 154-155° C.

### Reference Example 126

A mixture of N,O-dimethylhydroxylamine hydrochloride (11.4 g), triethylamine (11.9 g) and N,N-dimethylformamide (300 ml) was stirred at room temperature for 30 min, 3-butoxy-1-(2,4-dichlorobenzyl)-1H-pyrazole-5-carboxylic acid (33.6 g), 1-ethyl-3-(3-dimethylaminopropyl)carbodiimide hydrochloride (22.5 g) and 1-hydroxybenzotriazole monohydrate (18.0 g) were added, and the mixture was stirred at room temperature for 15 hr. The reaction mixture was concentrated. The residue was dissolved in ethyl acetate, and the mixture was washed with water, 1N hydrochloric acid, an aqueous potassium carbonate solution and saturated brine, dried (MgSO₄), and concentrated. The residue was subjected to silica gel column chromatography, and eluted with ethyl acetate-hexane (1:8-1:2, v/v) to give 3-butoxy-1-(2,4-dichlorobenzyl)-N-methoxy-N-methyl-1H-pyrazole-5-carboxamide (37.4 g, yield: 99%) as a colorless oil.

¹H-NMR (300 MHz, CDCl₃) δ:0.96 (3H, t, J=7.2 Hz), 1.40-1.53 (2H; m), 1.69-1.79 (2H, m), 3.28 (3H, s), 3.63 (3H, s), 4.14 (2H, t, J=6.6 Hz), 5.59 (2H, s), 6.24 (1H, s), 6.60 (1H, d, J=8.1 Hz), 7.11 (1H, dd, J=2.1, 8.4 Hz), 7.35 (1H, d, J=2.1 Hz).

### Reference Example 127

To a solution of 3-butoxy-1-(2,4-dichlorobenzyl)-N-methoxy-N-methyl-1H-pyrazole-5-carboxamide (37.4 g) in tetrahydrofuran (500 ml) was added a 1.5 M solution (97 ml) of diisobutylaluminum hydride in toluene at 0° C., and the mixture was stirred for 1 hr. Sodium sulfate 10 hydrate (50.0 g) was added to quench the reaction. The reaction mixture was stirred at room temperature for 5 hr, the insoluble material was filtrated, and the filtrate was concentrated. The residue was subjected to silica gel column chromatography, and eluted with ethyl acetate-hexane (1:20-1:10, v/v) to give 3-butoxy-1-(2,4-dichlorobenzyl)-1H-pyrazole-5-carbaldehyde (29.9 g, yield: 94%) as a colorless oil.

¹H-NMR (300 MHz, CDCl₃) δ:0.97 (3H, t, J=7.2 Hz), 1.40-1.53 (2H, m), 1.70-1.80 (2H, m), 4.15 (2, H t, J=6.6 Hz), 5.64 (2H, s), 6.33 (1H, s), 6.59 (1H, d, J=8.1 Hz), 7.12 (1H, d, J=2.1, 8.1 Hz), 7.39 (1H, d, J=8.1 Hz), 9.74 (1H, s).

### Reference Example 128

Under ice-cooling, to a solution of ethyl diethylphosphonoacetate (10.3° g) in N,N-dimethylformamide (50 ml) was added sodium hydride (60% in oil, 1.59 g), and the mixture was stirred at room temperature for 30 min. The reaction mixture was ice-cooled, a solution of 3-butoxy-1-(2,4-dichlorobenzyl)-1H-pyrazole-5-carbaldehyde (10.0 g) in tetrahydrofuran (30 ml) was added, and the mixture was stirred for 1 hr. The mixture was warmed to room temperature and further stirred for 30 min. The reaction mixture was concentrated, a saturated aqueous ammonium chloride solution was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:100-1:4, v/v) to give ethyl (2E)-3-[3-butoxy-1-(2,4-dichlorobenzyl)-1H-pyrazol-5-yl]acrylate (12.1 g, yield: 100%) as a yellow, oil.

¹H-NMR (300 MHz, CDCl₃) δ:0.96 (3H, t, J=7.4 Hz), 1.30 (3H, t, J=7.2 Hz), 1.40-1.55 (2H, m), 1.69-1.80 (2H, m), 4.12 (2H, t, J=6.6 Hz), 4.22 (2H, q, J=7.2 Hz), 5.33 (2H, s), 6.02 (1H, s), 6.28 (1H, d, J=15.8 Hz), 6.65 (1H, d, J=8.4 Hz), 7.15 (1H, dd, J=2.1, 8.4 Hz), 7.39 (1H, d, J=15.8 Hz), 7.40 (1H, d, J=2.1 Hz).

### Reference Example 129

A mixture of ethyl (2E)-3-[3-butoxy-1-(2,4-dichlorobenzyl)-1H-pyrazol-5-yl]acrylate (8.00 g), 5% palladium-carbon (1.00 g) and tetrahydrofuran (100 ml) was hydrogenated at room temperature under atmospheric pressure. The reaction mixture was filtrated, and the filtrate was concentrated to give ethyl 3-[3-butoxy-1-(2,4-dichlorobenzyl)-1H-pyrazol-5-yl]propanoate (8.01 g, yield: 60%) as a pale-yellow oil.

¹H-NMR (300 MHz, CDCl₃) δ:0.95 (3H, t, J=7.3 Hz), 1.24 (3H, t, J=7.2 Hz), 1.38-1.54 (2H, m), 1.66-1.78 (2H, m), 2.52-2.64 (2H, m), 2.72-2.82 (2H, m), 4.09 (2H, t, J=6.7 Hz), 4.12 (2H, q, J=7.2 Hz), 5.19 (2H, s), 5.54 (1H, s), 6.54 (1H, d, J=8.3 Hz), 7.15 (1H, dd, J=2.1, 8.3 Hz), 7.38 (1H, d, J=2.1 Hz).

### Reference Example 130

To a solution (100 ml) of 2-hydroxy-4-(methoxymethoxy)benzaldehyde (11.9 g) in N,N-dimethylformamide were added benzyl bromide (8.5 ml) and potassium carbonate (10.8 g) at room temperature, and the mixture was stirred at 80° C. for 1 hr. Water was poured into the reaction mixture, and the mixture was extracted with ethyl acetate. The ethyl acetate layer was washed with water and saturated brine, dried (MgSO₄), and concentrated to give an oil. To a solution (150 ml) of ethyl diethylphosphonoacetate (15.0 ml) in tetrahydrofuran was added sodium hydride (60% in oil, 3.3 g) at room temperature, and the mixture was stirred for 30 min. A solution (50 ml) of the oil in tetrahydrofuran obtained earlier was added dropwise to this reaction mixture, and the mixture was stirred at room temperature for 1 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:5, v/v) to give ethyl (2E)-3-[2-(benzyloxy)-4-(methoxymethoxy)phenyl]acrylate as a yellow oil (21.6 g, yield: 96%).

¹H-NMR (300 MHz, CDCl₃) δ:1.32 (3H, t, J=7.2 Hz), 3.46 (3H, s), 4.23 (2H, q, J=7.2 Hz), 5.14 (2H, s), 5.16 (2H, s), 6.44 (1H, d, J=16.2 Hz), 6.65-6.69 (2H, m), 7.28-7.49 (6H, m), 8.00 (1H, d, J=16.2 Hz).

### Reference Example 131

To an ethyl (2E)-3-[2-(benzyloxy)-4-(methoxymethoxy)phenyl]acrylate (4.0 g) in tetrahydrofuran-ethanol mixed solution (1:1, v/v, 100 ml) was added 10% palladium-carbon (0.5 g), and the mixture was stirred under a hydrogen atmosphere for 3 hr. The reaction mixture was filtrated, and the filtrate was concentrated. The residue was subjected to silica gel column chromatography, eluted with ethyl acetate-hexane (1:5, v/v) to give ethyl 3-[2-hydroxy-4-(methoxymethoxy)phenyl]propanoate as a colorless oil (2.8 g, yield: 95%).

¹H-NMR (300 MHz, CDCl₃) δ:1.24 (3H, t, J=7.2 Hz), 2.62-2.71 (2H, m), 2.78-2.89 (2H, m), 3.46 (3H, s), 4.15 (2H, q, J=7.2 Hz), 5.12 (2H, s), 6.52-6.64 (2H, m), 6.97 (1H, d, J=8.3 Hz), 7.42 (1H, s)

### Reference Example 132

To a solution (30 ml) of ethyl 3-[2-hydroxy-4-(methoxymethoxy)phenyl]propanoate (1.27 g) in N,N-dimethylformamide was added sodium hydride (60% in oil, 240 mg) at room temperature, and the mixture was stirred for 30 min. 2,3-Dichloro-5-(trifluoromethyl)pyridine (0.80 ml) was added to the reaction mixture, and the mixture was stirred at room temperature for 3 hr. Water was poured into the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (1:10, v/v) to give ethyl 3-[2-{[3-chloro-5-(trifluoromethyl)pyridin-2-yl]oxy}-4-(methoxymethoxy)phenyl]propanoate as a colorless oil (1.60 g, yield: 74%).

¹H-NMR (300 MHz, CDCl₃) δ:1.21 (3H, t, J=7.2 Hz), 2.52-2.64 (2H, m), 2.71-2.83 (2H, m), 3.47 (3H, s), 4.09 (2H, q, J=7.2 Hz), 5.15 (2H, s), 6.82 (1H, d, J=2.6 Hz), 6.93 (1H, dd, J=8.5, 2.5 Hz), 7.24 (1H, d, J=8.5 Hz), 7.97-8.00 (1H, m), 8.22-8.30 (1H, m).

### Reference Example 133

To a solution of 2-chloro-4-trifluoromethylbenzyl alcohol (38.8 g) and pyridine (3.0 ml) in diethyl ether (320 ml)-tetrahydrofuran (80 ml) was added thionyl chloride (32.8 g), and the mixture was stirred at room temperature for 15 hr. The reaction solution was concentrated, water was poured into the residue and the mixture was extracted with ethyl acetate. The ethyl acetate layer was washed with 1N hydrochloric acid, a saturated aqueous sodium hydrogencarbonate solution and saturated brine, dried (MgSO₄), and concentrated. The residue was subjected to silica gel column chromatography, and eluted with ethyl acetate-hexane (1:25-1:12, v/v) to give 2-chloro-4-trifluoromethylbenzyl chloride (38.9 g, yield: 92%) as a colorless oil.

¹H-NMR (300 MHz, CDCl₃) δ:4.72 (2H, s), 7.51-7.57 (1H, m), 7.60-7.70 (2H, m).

### Reference Example 134

A solution (50 ml) of ethyl (2E)-3-[2-(benzyloxy)-4-hydroxyphenyl]acrylate (5.1 g) in N,N-dimethylformamide was added 2-iodopropane (2.0 ml) and potassium carbonate (3.1 g), and the mixture was stirred at 60° C. for 1.5 hr. Water was poured into the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:5, v/v) to give ethyl (2E)-3-[2-(benzyloxy)-4-isopropoxyphenyl]acrylate as a colorless oil (5.0 g, yield: 86%).

¹H-NMR (300 MHz, CDCl₃) δ:1.27-1.35 (9H, m), 4.23 (2H, q, J=7.0 Hz), 4.46-4.61 (1H, m), 5.13 (2H, s), 6.38-6.54 (3 H, m), 7.28-7.51 (6H, m), 8.00 (1H, d, J=16.2 Hz).

### Reference Example 135

To ethyl (2E)-3-[2-(benzyloxy)-4-isopropoxyphenyl]acrylate (5.0 g) in a tetrahydrofuran-ethanol mixed solution (1:1, v/v, 100 ml) was added 10% palladium-carbon (0.5 g), and the mixture was stirred under a hydrogen atmosphere for 2 hr. The reaction mixture was filtrated, and the filtrate was concentrated. The residue was subjected to silica gel column chromatography, and eluted with ethyl acetate-hexane (1:4, v/v) to give ethyl 3-(2-hydroxy-4-isopropoxyphenyl)propanoate as a pale yellow oil (3.3 g, yield: 89%).

¹H-NMR (300 MHz, CDCl₃) δ:1.24 (3H, t, J t=7.2 Hz), 1.31 (6H, d, J=6.0 Hz), 2.63-2.72 (2H, m), 2.76-2.86 (2H, m), 4.15 (2H, q, J=7.2 Hz), 4.40-4.55 (1H, m), 6.38-6.48 (2H, m), 6.94 (1H, d, J=8.3 Hz), 7.43 (1H, s).

### Reference Example 136

To a solution (60 ml) of ethyl 3-(2-hydroxy-4-isopropoxyphenyl)propanoate (1.51 g) in N,N-dimethylformamide was added sodium hydride (60% in oil, 312 mg) at room temperature, and the mixture was stirred for 30 min. 2,3-Dichloro-5-(trifluoromethyl)pyridine (1.56 g) was added to this reaction mixture, and the mixture was stirred at 60° C. for 15 min. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:15, v/v) to give ethyl 3-(2-{[3-chloro-5-(trifluoromethyl)pyridin-2-yl]oxy}-4-isopropoxyphenyl)propanoate as a colorless oil (2.02 g, yield: 78%).

¹H-NMR (300 MHz, CDCl₃) δ:1.21 (3H, t, J=7.2 Hz), 1.32 (6H, d, J=6.0 Hz), 2.52-2.62 (2H, m), 2.70-2.81 (2H, m), 4.09 (2H, q, J=7.2 Hz), 4.41-4.56 (1H, m), 6.63 (1H, d, J=2.6 Hz), 6.76 (1H, dd, J=8.5, 2.6 Hz), 7.21 (1H, d, J=8.5 Hz), 7.98 (1H, d, J=2.1 Hz), 8.27 (1H, dd, J=2.1, 0.9 Hz).

### Reference Example 137

To a mixture of ethyl (2E)-3-[2-(benzyloxy)-4-hydroxyphenyl]acrylate (2.5 g), 2-methoxyethanol (1.0 ml), tributylphosphine (3.5 ml) and tetrahydrofuran (100 ml) was added 1,1′-azodicarbonyldipiperidine (4.2 g) at room temperature, and the mixture was stirred for 2 hr. The reaction mixture was concentrated, diisopropyl ether was added, and the precipitated crystals were filtered off. The filtrate was concentrated, and the obtained residue was subjected to silica gel column chromatography and eluted with ethyl acetate-hexane (1:5, v/v) to give ethyl (2E)-3-[2-(benzyloxy)-4-(2-methoxyethoxy)phenyl]acrylate as a colorless oil (2.5 g, yield: 83%).

¹H-NMR (300 MHz, CDCl₃) δ:1.31 (3H, t, J=7.2 Hz), 3.44 (3H, s), 3.69-3.78 (2H, m), 4.06-4.15 (2H, m), 4.23 (2H, q, T=7.2 Hz), 5.13 (2H, s), 6.37-6.58 (3H, m), 7.28-7.51 (6H, m), 7.99 (1H, d, J=16.2 Hz).

### Reference Example 138

To ethyl (2E)-3-[2-(benzyloxy)-4-(2-methoxyethoxy)phenyl]acrylate (2.5 g) in a tetrahydrofuran-ethanol mixed solution (1:1, v/v, 100 ml) was added 10% palladium-carbon (0.25 g), and the mixture was stirred under a hydrogen atmosphere for 3 hr. The reaction mixture was filtrated, and the filtrate was concentrated. The residue was subjected to silica gel column chromatography, and eluted with ethyl acetate-hexane (1:5-1:3, v/v) to give ethyl 3-[2-hydroxy-4-(2-methoxyethoxy)phenyl]propanoate as a colorless oil (2.8 g, yield: 95%).

¹H-NMR (300 MHz, CDCl₃) δ:1.23 (3H, t, J=7.2 Hz), 2.62-2.70 (2H, m), 2.78-2.86 (2H, m), 3.44 (3H, s), 3.70-3.75 (2H, m), 4.04-4.09 (2H, m), 4.14 (2H, q, J=7.2 Hz), 6.42-6.52 (2H, m), 6.95 (1H, d, J=8.1 Hz), 7.42-7.50 (1H, m).

### Reference Example 139

To a solution (40 ml) of ethyl 3-[2-hydroxy-4-(2-methoxyethoxy)phenyl]propanoate (1.6 g) in N,N-dimethylformamide was added sodium hydride (60% in oil, 285 mg) at room temperature, and the mixture was stirred for 30 min. 2,3-Dichloro-5-(trifluoromethyl)pyridine (0.9 ml) was added to this reaction mixture, and the mixture was stirred at room temperature for 30 min. Water was poured into the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:5, v/v) to give ethyl 3-[2-{[3-chloro-5-(trifluoromethyl)pyridin-2-yl]oxy}-4-(2-methoxyethoxy)phenyl]propanoate as a colorless oil (2.2 g, yield: 84%).

¹H-NMR (300 MHz, CDCl₃) δ:1.21 (3H, t, J=7.2 Hz), 2.50-2.63 (2H, m), 2.72-2.84 (2H, m), 3.43 (3H, s), 3.65-3.79 (2H, m), 4.02-4.16 (4H, m), 6.68 (1H, d, J=2.6 Hz), 6.82 (1H, dd, J=8.5, 2.6 Hz), 7.22 (1H, d, J=8.5 Hz), 7.98° (1H d, J=1.9 Hz), 8.21-8.29 (1H, m).

### Reference Example 140

To a solution (100 ml) of ethyl 3-[2-{[3-chloro-5-(trifluoromethyl)pyridin-2-yl]oxy}-4-(2-methoxyethoxy)phenyl]propanoate (2.2 g) in tetrahydrofuran was added lithium aluminum hydride (186 mg) at room temperature, and the mixture was stirred for 15 min. Sodium sulfate 10 hydrate (1.6 g) was added to this reaction mixture, and the mixture was filtered through celite and concentrated. The residue was subjected to silica gel column chromatography, and eluted with ethyl acetate-hexane (1:2, v/v) to give 3-[2-{[3-chloro-5-(trifluoromethyl)pyridin-2-yl]oxy}-4-(2-methoxyethoxy)phenyl]propan-1-ol as a colorless oil (1.7 g, yield: 83%). Recrystallization from ethyl acetate-hexane gave colorless prism crystals. melting point 70° C.-71° C.

### Reference Example 141

To a solution of ethyl 3-(3-isopropoxy-1H-pyrazol-5-yl)propionate (1.50 g) in N,N-dimethylformamide (20 ml) was added sodium hydride (60% in oil, 290 mg) at room temperature, and the mixture was stirred for 10 min. 2,4-Difluorobenzylbromide (1.65 g) was added to the reaction mixture, and the mixture was stirred at room temperature for 4 hr. 1N Hydrochloric acid was added, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:49-1:3, v/v) to give ethyl 3-[1-(2,4-difluorobenzyl)-3-isopropoxy-1H-pyrazol-5-yl]propionate (660 mg, yield: 28%) as a colorless oil.

¹H-NMR (300 MHz, CDCl₃) δ:1.24 (3H, t, J=7.2 Hz), 1.32 (6H, d, J=6.3 Hz), 2.52-2.62 (2H, m), 2.77-2.87 (2H, m), 4.12 (2H, q, J=7.2 Hz), 4.60-4.76 (1H, m), 5.13 (2H, s), 5.47 (1H, s), 6.66-6.97 (3H, m).

### Reference Example 142

A mixture of methyl 3-isopropoxy-1H-pyrazole-5-carboxylate (11.08 g), 2,4-dichlorobenzyl chloride (13.0 g), potassium carbonate (10.00 g) and N,N-dimethylformamide (80 ml) was stirred overnight at room temperature. Water (100 ml) was added to the reaction mixture, and the mixture was extracted with ethyl acetate (100 ml×²). The organic layer was washed with brine, dried (MgSO₄), filtrated and concentrated. The residue was subjected to silica gel column chromatography, and eluted with ethyl acetate-hexane (1:8-1:2, v/v) to give methyl 1-(2,4-dichlorobenzyl)-3-isopropoxy-1H-pyrazole-5-carboxylate (Reference Example 142a) (13.68 g, yield: 66%) as a colorless oil.

¹H-NMR (300 MHz, CDCl₃) δ:1.34 (6H, d, J=5.8 Hz), 3.81 (3H, s), 4.72 (1H, septet, J=6.2 Hz), 5.67 (2H, s), 6.28 (1H, s), 6.54 (1H, d, J=8.4 Hz), 7.12 (1H, dd, J=8.4, 1.8 Hz), 7.38 (1H, d, J=2.2 Hz).

Then, methyl 1-(2,4-dichlorobenzyl)-5-isopropoxy-1H-pyrazole-3-carboxylate (Reference Example 142b) (6.00 g, yield: 29%) was obtained as colorless crystals. melting point 89-90° C.

### Reference Example 143

A solution of methyl 1-(2,4-dichlorobenzyl)-3-isopropoxy-1H-pyrazole-5-carboxylate (13.68 g) in tetrahydrofuran (40 ml) was added to a suspension of lithium aluminum hydride (1.90 g) in tetrahydrofuran (70 ml) under ice-cooling over 45 min, and the mixture was stirred for 30 min. Ethanol (20 ml) and then a saturated aqueous ammonium chloride solution (7.0 ml) were added to the reaction mixture, the precipitated inorganic product was filtered off, and washed with acetone. Ethyl acetate (100 ml) was added to the residue, and the mixture was dried (MgSO₄), filtrated and concentrated to give [1-(2, 4-dichlorobenzyl)-3-isopropoxy-1H-pyrazol-5-yl]methanol as colorless crystals (12.05 g, yield: 96%) melting point 89-90° C.

### Reference Example 144

A solution of dimethyl sulfoxide (10.8 ml) in dichloromethane (100 ml) was cooled to −70° C., oxalyl chloride (6.70 ml) was added over 20 min, and the mixture was stirred for 20 min. Then a solution of [1-(2,4-dichlorobenzyl)-3-isopropoxy-1H-pyrazol-5-yl]methanol (12.05 g) in dichloromethane (15 ml) was added over 20 min, and the mixture was stirred for 30 min. Triethylamine (29 ml) was added to the reaction mixture over 10 min, and the mixture was gradually warmed to room temperature. 1N Hydrochloric acid (100 ml) was added to the reaction mixture, the dichloromethane layer was separated and concentrated to give a residue. Separately, the aqueous layer was extracted with ethyl acetate (100 ml) and combined with the residue obtained earlier. The organic layer was washed with brine, dried (MgSO₄), filtrated and concentrated. The residue was subjected to silica gel column chromatography, and eluted with ethyl acetate-hexane (1:6, v/v) to give 1-(2,4-dichlorobenzyl)-3-isopropoxy-1H-pyrazole-5-carbaldehyde as a yellow oil (11.37 g, yield: 95%).

¹H-NMR (300 MHz, CDCl₃) δ:1.35 (6H, d, J=6.2 Hz), 4.76 (1H, septet, J=6.1 Hz), 5.64 (2H, s), 6.31 (1H, s), 6.60 (1H, d, J=8.4 Hz), 7.13 (1H, dd, J=8.4, 2.2 Hz), 7.39 (1H, d, J=2.2 Hz), 9.74 (1H, s).

### Reference Example 145

A solution of 1-(2,4-dichlorobenzyl)-3-isopropoxy-1H-pyrazole-5-carbaldehyde (11.37 g) and ethyl diethylphosphonoacetate (8.95 g) in tetrahydrofuran (40 ml) was added to a suspension of sodium hydride (60% in oil, 1.75 g) in N,N-dimethylformamide (140 ml) under ice-cooling over 40 min, and the mixture was stirred at room temperature for 30 min. Water (200 ml) was added to the reaction mixture, and the mixture was extracted with ethyl acetate (100 ml×²). The organic layer was washed with brine, dried (MgSO₄), filtrated and concentrated. The residue was subjected to silica gel column chromatography, and eluted with ethyl acetate-hexane (1:6, v/v) to give ethyl (2E)-3-[1-(2,4-dichlorobenzyl)-3-isopropoxy-1H-pyrazol-5-yl]propenoate as a yellow oil (12.78 g, yield: 92%).

¹H-NMR (300 MHz, CDCl₃) δ:1.30 (3H, t, J=6.9 Hz), 1.34 (6H, d, J=6.2 Hz), 4.23 (2H, q, J=7.2 Hz), 4.72 (1H, septet, J=6.4 Hz), 5.33 (2H, s), 6.00 (1H, s), 6.27 (1H, d, J=15.9 Hz), 6.66 (1H, d, J=8.4 Hz), 7.15 (1H, dd, J=8.2, 1.8 Hz), 7.35-7.45 (2H, m).

### Reference Example 146

A mixture of ethyl (2E)-3-[1-(2,4-dichlorobenzyl)-3-isopropoxy-1H-pyrazol-5-yl]propenoate (12.78 g), 5% palladium-carbon (1.0 g) and tetrahydrofuran (80 ml) was stirred at room temperature under a hydrogen atmosphere for 6 hr. The catalyst was filtered off, and the filtrate was concentrated. The residue was subjected to silica gel column chromatography, and eluted with ethyl acetate-hexane (1:6, v/v) to give ethyl 3-[1-(2,4-dichlorobenzyl)-3-isopropoxy-1H-pyrazol-5-yl]propionate as a pale-yellow oil (11.37 g, yield: 89%).

¹H-NMR (300 MHz, CDCl₃) δ:1.24 (3H, t, J=7.2 Hz), 1.33 (6H, d, J=6.2 Hz), 2.47-2.64 (2H, m), 2.69-2.85 (2H, m), 4.12 (2H, q, J=7.2 Hz), 4.63-4.80 (1H, m), 5.18 (2H, s), 5.52 (1H, s), 6.56 (1H, d, J=8.5 Hz), 7.15 (1H, dd, J=8.4, 2.2 Hz), 7.38 (1H, d, J=2.1 Hz).

### Reference Example 147

A mixture of ethyl 3-(3-isopropyl-1H-pyrazol-5-yl)propionate (1.22 g), 2,4-dichlorobenzyl chloride (0.82 ml), potassium carbonate (0.75 g) and N,N-dimethylformamide (10 ml) was stirred overnight at 80° C. Water (15 ml) was added to the reaction mixture, and the mixture was extracted with ethyl acetate (20 ml×2). The organic layer was washed with brine, dried (MgSO₄), filtrated and concentrated. The residue was subjected to silica gel column chromatography, and eluted with ethyl acetate-hexane (1:7, v/v) to give ethyl 3-[1-(2,4-dichlorobenzyl)-3-isopropoxy-1H-pyrazol-5-yl]propionate as a yellow oil (1.12 g, yield: 54%).

¹H-NMR (300 MHz, CDCl₃) δ:1.24 (3H, t, J=7.1 Hz), 1.33 (6H, d, J=5.8 Hz), 2.50-2.63 (2H, m), 2.70-2.82 (2H, m), 4.12 (2H, q, J=7.2 Hz), 4.70 (1H, septet, J=6.2 Hz), 5.18 (2H, s), 5.52 (1H, s), 6.56 (1H, d, J=8.0 Hz), 7.15 (1H, dd, J=8.4, 2.2 Hz), 7.38 (1H, d, J=2.2 Hz).

### Reference Example 148

To a 1N solution of borane in tetrahydrofuran (30 ml) was added 2-chloro-4-trifluoromethylbenzoic acid (2.50 g), and the mixture was stirred at room temperature for 1 hr. The reaction mixture was poured into 1N hydrochloric acid, and the mixture was extracted with ethyl acetate. The extract was washed with an aqueous sodium hydrogencarbonate solution and saturated brine, dried (MgSO₄), and concentrated. The residue was subjected to silica gel column chromatography, and eluted with ethyl acetate-hexane (1:24-1:4, v/v) to give 2-chloro-4-trifluoromethylbenzyl alcohol (2.23 g, yield: 96%) as a colorless oil.

¹H-NMR (300 MHz, CDCl₃) δ:4.85 (2H, d, J=9.0 Hz), 7.52-7.80 (3H, m).

### Reference Example 149

To a solution of 2-chloro-4-trifluoromethylbenzyl alcohol (2.23 g) and triphenylphosphine (4.17 g) in tetrahydrofuran (25 ml) was added carbon tetrabromide (5.27 g), and the mixture was stirred at room temperature for 2 hr. The reaction solution was concentrated, hexane and diethyl ether was added to the residue, and the insoluble material was filtered off. The mother liquor was concentrated, and the obtained residue was subjected to silica gel column chromatography and eluted with ethyl acetate-hexane (0:1-1:4, v/v) to give 2-chloro-4-trifluoromethylbenzylbromide (2.90 g, yield: 99%) as a colorless oil.

¹H-NMR (300 MHz, CDCl₃) δ:4.59 (2H, s), 7.46-7.60 (2H, m), 7.65 (1H, s).

### Reference Example 150

To a solution of ethyl 3-(3-isopropoxy-1H-pyrazol-5-yl)propionate (1.50 g) in N,N-dimethylformamide (20 ml) was added sodium hydride (60% in oil, 290 mg) at room temperature, and the mixture was stirred for 10 min. 2-Chloro-4-trifluoromethylbenzylbromide (2.17 g) was added to the reaction mixture, and the mixture was stirred at room temperature for 4 hr. 1N Hydrochloric acid was added, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:49-1:4, v/v) to give ethyl 3-{1-[2-chloro-4-(trifluoromethyl)benzyl]-3-isopropoxy-1H-pyrazol-5-yl}propionate (610 mg, yield: 22%) as a yellow oil.

¹H-NMR (300 MHz, CDCl₃) δ:1.23 (3H, t, J=7.2 Hz), 1.32 (6H, d, J=6.3 Hz), 2.53-2.62 (2H, m), 2.73-2.80 (2H, m), 4.11 (2H, q, J=7.2 Hz), 4.64-4.76 (1H, m), 5.25 (2H, s), 5.54 (1H, s), 6.69 (1H, d, J=7.8 Hz), 7.41 (1H, d, J=7.8 Hz), 7.62 (1H, s).

### Reference Example 151

To a mixture of potassium tert-butoxide (55.1 g) in tetrahydrofuran (300 ml) was added a mixture of 3-methylbutan-2-one (30.0 g) and diethyl oxalate (51.0 g) at room temperature over 1 hr. The mixture was stirred overnight at room temperature, acetic acid (47.7 ml) and hydrazine monohydrate (19.0 g) were added, and the mixture was stirred with heating under reflux for 2 hr. The reaction mixture was concentrated, water (200 ml) was added, and the mixture was extracted with ethyl acetate (150 ml×²). The organic layer was washed with brine, dried (MgSO₄), filtrated and concentrated. The residue was subjected to silica gel column chromatography, and eluted with ethyl acetate-hexane (1:1, v/v) to give ethyl 3-isopropyl-1H-pyrazole-5-carboxylate as a brown oil (31.82 g, yield: 50%).

¹H-NMR (300 MHz, CDCl₃) δ:1.30 (6H, d, J=7.0 Hz), 1.38 (3H, t, J=7.2 Hz), 3.04 (1H, septet, J=6.8 Hz), 4.37 (2H, q, J=6.8 Hz), 4.37 (2H, q, J=7.1 Hz), 6.63 (1H, d, J=0.6 Hz).

### Reference Example 152

Ethyl 3-isopropyl-1H-pyrazole-5-carboxylate (10.00 g), 2,4-dichlorobenzyl chloride (11.8 g), potassium carbonate (9.0 g) and N,N-dimethylformamide (50 ml) were stirred overnight at room temperature. Water (80 ml) was added to the reaction mixture, and the mixture was extracted with ethyl acetate (50 ml×2.). The organic layer was washed with brine, dried (MgSO₄), filtrated and concentrated. The residue was subjected to silica gel column chromatography, and eluted with ethyl acetate-hexane (1:15-1:4, v/v) to give ethyl 1-(2,4-dichlorobenzyl)-3-isopropyl-1H-pyrazole-5-carboxylate (Reference Example 152a) as a brown oil (10.52 g, yield: 56%).

¹H-NMR (300 MHz, CDCl₃) δ:1.29 (6H, d, J=6.8 Hz), 1.29 (3H, t, J=7.1 Hz), 3.02 (1H, septet, J=6.9 Hz), 4.26 (2H, q,

J=7.1 Hz), 5.77 (2H, s), 6.34 (1H, d, J=8.6 Hz), 6.78 (1H, s), 7.09 (1H, dd, J=8.4, 2.2 Hz), 7.38 (1H, d, J=2.2 Hz).

Then, ethyl 1-(2,4-dichlorobenzyl)-5-isopropyl-1H-pyrazole-3-carboxylate (Reference Example 152b) (8.73 g, yield: 47%) was obtained.

¹H-NMR (300 MHz, CDCl₃) δ:1.18 (6H, d, J=7.0 Hz), 1.40 (3H, t, J=7.1 Hz), 2.79 (1H, septet, J=6.8 Hz), 4.42 (2H, q, J=7.2 Hz), 5.46 (2H, s), 6.49 (1H, d, J=8.0 Hz), 6.70 (1H, s), 7.13 (1H, dd, J=8.4, 2.2 Hz), 7.40 (1H, d, J=1.8 Hz).

### Reference Example 153

To a solution of ethyl 1-(2,4-dichlorobenzyl)-3-isopropyl-1H-pyrazole-5-carboxylate (10.52 g) in tetrahydrofuran (100 ml) was added a 1.5 M solution (51.5 ml) of diisobutylaluminum hydride in toluene over 30 min under ice-cooling. The mixture was stirred at room temperature for 30 min, and ethanol (30 ml) and then a saturated aqueous ammonium chloride solution (13 ml) were added to the reaction solution. The precipitated inorganic substance was filtered off, and washed with acetone. The filtrate was concentrated, and the obtained residue was subjected to silica gel column chromatography, and eluted with ethyl acetate-hexane (1:2, v/v) to give [1-(2,4-dichlorobenzyl)-3-isopropyl-1H-pyrazol-5-yl]methanol as colorless crystals (7.90 g, yield: 86%) melting point 89-90° C.

### Reference Example 154

A solution of dimethyl sulfoxide (7.50 ml) in dichloromethane (100 ml) was cooled to −70° C., oxalyl chloride (4.60 ml) was added over 15 min, and the mixture was stirred for 15 min. Then, a solution of [1-(2,4-dichlorobenzyl)-3-isopropyl-1H-pyrazol-5-yl]methanol (7.90 g) in dichloromethane (25 ml) was added over 20 min, and the mixture was stirred for 20 min. Triethylamine (20.0 ml) was added to the reaction mixture over 10 min, and the mixture was slowly warmed to room temperature. The reaction mixture was concentrated, and the precipitated salt was filtered off, and washed with diisopropyl ether. The filtrate was concentrated, and the obtained residue was subjected to silica gel column chromatography and eluted with ethyl acetate-hexane (1:15-1:6, v/v) to give 1-(2,4-dichlorobenzyl)-3-isopropyl-1H-pyrazole-5-carbaldehyde as a yellow oil (7.08 g, yield: 90%).

¹H-NMR (300 MHz, CDCl₃) δ:1.30 (6H, d, J=7.0 Hz), 3.05 (1H, septet, J=6.9 Hz), 5.75 (2H, s), 6.42 (1H, d, J=8.6 Hz), 6.81 (1H, s), 7.10 (1H, dd, J=8.2, 2.0 Hz), 7.39 (1H, d, J=2.2 Hz), 9.79 (1H, s).

### Reference Example 155

A solution of 1-(2,4-dichlorobenzyl)-3-isopropyl-1H-pyrazole-5-carbaldehyde (7.08 g) and ethyl diethylphosphonoacetate (5.87 g) in tetrahydrofuran (15 ml) was added to a suspension of sodium hydride (60% in oil, 1.15 g) in N,N-dimethylformamide (40 ml) under ice-cooling over 20 min, and the mixture was stirred at room temperature for 1 hr. Water (80 ml) was added to the reaction mixture, and the mixture was extracted with ethyl acetate (40 ml×2). The organic layer was washed with brine, dried (MgSO₄), filtrated and concentrated. The residue was subjected to silica gel column chromatography, and eluted with ethyl acetate-hexane (1:4, v/v) to give ethyl (2E)-3-[1-(2,4-dichlorobenzyl)-3-isopropyl-1H-pyrazol-5-yl]propenoate as a yellow oil (7.07 g, yield: 81%).

¹H-NMR (300 MHz, CDCl₃) δ:1.22-1.37 (9H, m), 2.99 (1H, septet, J=6.9 Hz), 4.22 (2H, q, J=7.2 Hz), 5.44 (2H, s), 6.30 (1H, d, J=15.8 Hz), 6.44-6.53 (2H, n), 7.13 (1H, dd, J=8.2, 2.0 Hz), 7.40 (1H, d, J=2.2 Hz), 7.41 (1H, d, J=15.8 Hz).

### Reference Example 156

A mixture of ethyl (2E)-3-[1-(2,4-dichlorobenzyl)-3-isopropyl-1H-pyrazol-5-yl]propenoate (7.07 g), 5% palladium-carbon (1.4 g) and tetrahydrofuran (45 ml) was stirred at room temperature under a hydrogen atmosphere for 4 hr. The catalyst was filtered off, and the filtrate was concentrated. The residue was subjected to silica gel column chromatography, and eluted with ethyl acetate-hexane (1:6, v/v) to give ethyl 3-[1-(2,4-dichlorobenzyl)-3-isopropyl-1H-pyrazol-5-yl]propionate as a colorless oil (5.36 g, yield: 76%).

¹H-NMR (300 MHz, CDCl₃): 1.20-1.32 (9H, m), 2.53-2.65 (2H, m), 2.72-2.85 (2H, m), 2.96 (1H, septet, J=7.0 Hz), 4.13 (2H, q, J=7.2 Hz), 5.30 (2H, s), 5.94 (1H, s), 6.38 (1H, d, J=8.4 Hz), 7.13 (1H, dd, J=8.4, 2.2 Hz), 7.38 (1H, d, J=2.2 Hz).

### Reference Example 157

To a solution of ethyl 3-isopropyl-1H-pyrazole-5-carboxylate (4.39 g) and 2-chloro-4-trifluorobenzyl chloride (6.07 g) in N,N-dimethylformamide (70 ml) was added potassium carbonate (3.99 g), and the mixture was stirred at room temperature for 15 hr. The reaction mixture was poured into water, and the mixture was extracted with ethyl acetate. The extract was washed with water and saturated brine, dried (MgSO₄), and concentrated. The residue was subjected to silica gel column chromatography, and eluted with ethyl acetate-hexane (1:49-1:4, v/v) to give ethyl 1-[2-chloro-4-(trifluoromethyl)benzyl]-3-isopropyl-1H-pyrazole-5-carboxylate (3.80 g, yield: 42%) as a colorless oil.

¹H-NMR (300 MHz, CDCl₃) δ:1.29 (3H, t, J=7.1 Hz), 1.30 (6H, d, J=7.0 Hz), 2.91-3.10 (1H, m), 4.26 (2H, q, J=7.1 Hz), 5.85 (2H, s), 6.48 (1H, d, J=8.1 Hz), 6.80 (1H, s), 7.38 (1H, d, J=8.1 Hz), 7.64 (1H, s).

### Reference Example 158

To a solution of ethyl 1-[2-chloro-4-(trifluoromethyl)benzyl]-3-isopropyl-1H-pyrazole-5-carboxylate (3.80 g) in tetrahydrofuran (50 ml) was added a 1.5 M solution (17 ml) of diisobutylaluminum hydride in toluene at DOC. After stirring at room temperature for 1 hr, methanol was added to quench the reaction. A 10% Rochelle salt aqueous solution (200 ml) was added, and the mixture was stirred for 4 hr and extracted with diethyl ether. The extract was washed with saturated brine, dried (MgSO₄), and concentrated. The residue was subjected to silica gel column chromatography, and eluted with ethyl acetate-hexane (1:19-2:3, v/v) to give {1-[2-chloro-4-(trifluoromethyl)benzyl]-3-isopropyl-1H-pyrazol-5-yl}methanol (3.37 g, yield: 99%) as a pale-yellow oil.

¹H-NMR (300 MHz, CDCl₃) δ:1.29 (6H, d, J=6.9 Hz), 1.63 (1H, t, J=5.6 Hz), 2.92-3.06 (1H, m), 4.57 (1H, d, J=5.6 Hz), 5.49 (2H, s), 6.14 (1H, s), 6.59 (1H, d, J=8.0 Hz), 7.39 (1H, dd, J=1.2, 8.0 Hz), 7.62 (1H, d, J=1.2 Hz).

### Reference Example 159

Under a nitrogen atmosphere, to a solution of oxalyl chloride (2.56 g) in methylene chloride (50 ml) was added dimethyl sulfoxide (2.15 ml) at −78° C. After stirring at −78° C. for 5 min, a solution of {1-[2-chloro-4-(trifluoromethyl)benzyl]-3-isopropyl-1H-pyrazol-5-yl}methanol (3.37 g) in methylene chloride (35 ml) was added. After stirring at −78° C. for 1 hr, triethylamine (5.11 g) was added. The reaction mixture was stirred at room temperature for 1 hr and poured into water, and the mixture was extracted with ethyl acetate. The extract was washed with 1N hydrochloric acid, a saturated aqueous sodium hydrogencarbonate solution and saturated brine, dried (MgSO₄), and concentrated. The residue was subjected to silica gel column chromatography, and eluted with ethyl acetate-hexane (1:19-1:4, v/v) to give 1-[2-chloro-4-(trifluoromethyl)benzyl]-3-isopropyl-1H-pyrazole-5-carbaldehyde (3.32 g, yield: 99%) as a pale-yellow oil.

¹H-NMR (300 MHz, CDCl₃): 1.31 (6H, d, J=7.2 Hz), 2.98-3.13 (1H, m), 5.82 (2H, s), 6.52 (1H, d, J=8.1 Hz), 6.83 (1H, s), 7.36 (1H, dd, J=1.2, 8.1 Hz), 7.65 (1H, d, J=1.2 Hz), 9.79 (1H, s).

### Reference Example 160

Under ice-cooling, to a suspension of sodium hydride (60% in oil, 560 mg) in N,N-dimethylformamide (30 ml) was added a mixed solution of 1-[2-chloro-4-(trifluoromethyl)benzyl]-3-isopropyl-1H-pyrazole-5-carbaldehyde (3.32 g) and ethyl diethylphosphonoacetate (2.69 g) in tetrahydrofuran (15 ml). After stirring at room temperature for 1.5 hr. the reaction mixture was poured into water, and the mixture was extracted with ethyl acetate. The extract was washed with water and saturated brine, dried (MgSO₄), and concentrated. The residue was subjected to silica gel column chromatography, and eluted with ethyl acetate-hexane (1:49-3:17, v/v) to give ethyl (2E)-3-{1-[2-chloro-4-(trifluoromethyl)benzyl]-3-isopropyl-1H-pyrazol-5-yl}acrylate (1.18 g, yield: 29%) as a pale-yellow oil.

¹H-NMR (300 MHz, CDCl₃) δ:1.28 (6H, d, J=6.9 Hz), 1.30 (3H, t, J=7.2 Hz), 2.93-3.07 (1H, m), 4.21 (2H, q, J=7.2 Hz), 5.51 (2H, s), 6.31 (1H, d, J=15.8 Hz), 6.50 (1H, s), 6.60 (1H, d, J=7.8 Hz), 7.38 (1H, d, J=15.8 Hz), 7.40 (1H, d, J=7.8 Hz), 7.65 (1H, s)

### Reference Example 161

A mixture of ethyl (2E)-3-{1-[2-chloro-4-(trifluoromethyl)benzyl]-3-isopropyl-1H-pyrazol-5-yl}acrylate (1.18 g), 5% palladium-carbon (290 mg) and tetrahydrofuran (15 ml) was hydrogenated at room temperature under atmospheric pressure. The reaction mixture was filtrated, and the filtrate was concentrated to give ethyl 3-{1-[2-chloro-4-(trifluoromethyl)benzyl]-3-isopropyl-1H-pyrazol-5-yl}propionate (1.00 g, yield: 84%) as a pale-yellow oil.

¹H-NMR (300 MHz, CDCl₃) δ:1.23 (3H, t, J=7.2 Hz), 1.26 (6H, d, J=7.2 Hz), 2.57-2.64 (2H, m), 2.74-2.81 (2H, m), 2.89-3.02 (1H, m), 4.11 (2H, q, J=7.2 Hz), 5.38 (2H, s), 5.96 (1H, s), 6.51 (1H, d, J=8.1 Hz), 7.40 (1H, dd, J=1.2, 8.1 Hz), 7.63 (1H, d, J=1.2 Hz).

### Reference Example 162

To a mixture of ethyl (2E)-3-[2-hydroxy-4-(2-methoxyethoxy)phenyl]acrylate (0.98 g), cyclohexanol (0.48 g), tributylphosphine (0.89 g) and tetrahydrofuran (10 ml) was added 1,1′-(azodicarbonyl)dipiperidine (1.03 g) at room temperature, and the mixture was stirred for 6 hr. Tributylphosphine (0.89 g) and 1,1′-(azodicarbonyl)dipiperidine (1.03 g) were added to the reaction mixture at room temperature, and the mixture was stirred at 50° C. for 24 hr. The reaction mixture was concentrated, diisopropyl ether was added, and the precipitated crystals were filtered off. The filtrate was concentrated, and the obtained residue was subjected to silica gel column chromatography and eluted with ethyl acetate-hexane (3:17-3:7, v/v) to give ethyl (2E)-3-[2-(cyclohexyloxy)-4-(2-methoxyethoxy)phenyl]acrylate as a colorless oil (0.80 g, yield: 83%).

¹H-NMR (300 MHz, CDCl₃) δ:1.26-1.44 (7H, m), 1.46-1.70 (4H, m), 1.70-2.04 (4H, m), 3.45 (3H, s), 3.73-3.76 (2H, m), 4.11-4.14 (2H, m), 4.20-4.32 (3H, m), 6.38-6.52 (3H, m), 7.42 (1H, d, J=8.7 Hz), 7.93 (1H, d, J=16.2 Hz).

### Reference Example 163

A mixture of ethyl (2E)-3-[2-(cyclohexyloxy)-4-(2-methoxyethoxy)phenyl]acrylate (0.80 g), 10% palladium-carbon (0.15 g) and tetrahydrofuran (35 ml) was stirred under a hydrogen atmosphere at room temperature for 3 hr. The catalyst was filtered off, and the filtrate was concentrated to give ethyl 3-[2-(cyclohexyloxy)-4-(2-methoxyethoxy)phenyl]propanoate as a pale-black oil (0.80 g, yield: 99%).

¹H-NMR (300 MHz, CDCl₃) δ:1.23 (3H, t, J=7.2 Hz), 1.30-1.62 (6H, m), 1.70-1.98 (4H, m), 2.54-2.59 (2H, m), 2.83-2.88 (2H, m), 3.44 (3H, s), 3.71-3.74 (2H, m), 4.06-4.14 (4H, m), 4.20-4.28 (1H, m), 6.36 (1H, dd, J=8.4, 2.4 Hz), 6.48 (1H, d, J=2.4 Hz), 7.01 (1H, d, J=8.1 Hz).

### Reference Example 164

Ethyl 3-[2-(cyclohexyloxy)-4-(2-methoxyethoxy)phenyl]propanoate (0.80 g) was dissolved in tetrahydrofuran (20 ml), a 1.5 M diisobutylaluminum hydride solution in toluene (6.1 ml) was added at 0° C., and the mixture was stirred at room temperature for 1 hr. Sodium sulfate decahydrate (2.95 g) and diethyl ether (50 ml) were added to the reaction mixture, and the mixture was stirred overnight. The resulting solid was filtered off and the filtrate was concentrated. The obtained residue was subjected to silica gel column chromatography, and eluted with ethyl acetate-hexane (1:4-2:3, v/v) to give 3-[2-(cyclohexyloxy)-4-(2-methoxyethoxy)phenyl]propan-1-ol as a colorless oil (0.63 g, yield: 90%).

¹H-NMR (300 MHz, CDCl₃) δ:1.24-1.42 (2H, m), 1.44-1.60 (4H, m), 1.72-1.84 (4H, m), 1.96-2.00 (3H, m), 2.66 (2H, t, J=6.9 Hz), 3.45 (3H, s), 3.55 (2H, q, J=6.0 Hz), 3.72-3.75 (2H, m), 4.07-4.10 (2H, m), 4.20-4.28 (1H, m), 6.42 (1H, dd, J=8.1, 2.4 Hz), 6.52 (1H, d, J=2.4 Hz), 7.01 (1H, d, J=8.1 Hz).

### Reference Example 165

A mixture of ethyl (2E)-3-[2-(cyclopropylmethoxy)-4-(2-methoxyethoxy)phenyl]acrylate (1.18 g), 10% palladium-carbon (0.25 g) and tetrahydrofuran (50 ml) was stirred under a hydrogen atmosphere at room temperature for 1 hr. The catalyst was filtered off, and the filtrate was concentrated to give ethyl 3-[2-(cyclopropylmethoxy)-4-(2-methoxyethoxy)phenyl]propanoate as a colorless oil (1.18 g, yield: 98%).

¹H-NMR (300 MHz, CDCl₃) δ:0.32-0.34 (2H, m), 0.59-0.62 (2H, m), 1.21-1.30 (4H, m), 2.56-2.62 (2H, m), 2.89 (2H, t, J=7.8 Hz), 3.44 (3H, s), 3.71-3.74 (2H, m), 3.78 (2H, d, J=6.6 Hz), 4.07-4.15 (4H, m), 6.39 (1H, dd, J=8.1, 2.4 Hz), 6.45 (1H, d, J=2.4 Hz), 7.02 (1H, d, J=8.1 Hz).

### Reference Example 166

Ethyl 3-[2-(cyclopropylmethoxy)-4-(2-methoxyethoxy)phenyl]propanoate (1.18 g) was dissolved in tetrahydrofuran (30 ml), a 1.5 M diisobutylaluminum hydride solution in toluene (10 ml) was added at 0° C., and the mixture was stirred at room temperature for 40 min. Sodium sulfate decahydrate (4.83 g) and diethyl ether (60 ml) were added to the reaction mixture, and the mixture was stirred overnight. The resulting solid was filtered off and the filtrate was concentrated. The obtained residue was subjected to silica gel column chromatography, and eluted with ethyl acetate-hexane (3:7-9:11, v/v) to give 3-[2-(cyclopropylmethoxy)-4-(2-methoxyethoxy)phenyl]propan-1-ol as a colorless oil (0.99 g, yield: 94%).

¹H-NMR (300 MHz, CDCl₃) δ:0.32-0.38 (2H, m), 0.60-0.66 (2H, m), 1.22-1.32 (1H, m), 1.76-1.84 (2H, m), 2.14 (1H, t, J=6.3 Hz), 2.70 (2H, t, J=6.6 Hz), 3.44 (3H, s), 3.55 (2H, q, J=6.0 Hz), 3.71-3.75 (2H, m), 3.78 (2H, d, J=6.9 Hz), 4.07-4.10 (2H, m), 6.42-6.46 (2H, m), 7.01 (1H, d, J=8.1 Hz).

### Reference Example 167

A mixture of ethyl (2E)-3-[4-(2-methoxyethoxy)-2-(tetrahydrofuran-2-ylmethoxy)phenyl]acrylate (1.05 g), 10% palladium-carbon (0.20 g) and tetrahydrofuran (40 ml) was stirred under a hydrogen atmosphere at room temperature for 1 hr. The catalyst was filtered off, and the filtrate was concentrated to give ethyl 3-[4-(2-methoxyethoxy)-2-(tetrahydrofuran-2-ylmethoxy)phenyl]propanoate as a colorless oil (1.18 g, yield: 98%).

¹H-NMR (300 MHz, CDCl₃) δ:1.20-1.25 (3H, m), 1.80-2.12 (4H, m), 2.53-2.59 (2H, m), 2.84-2.90 (2H, m), 3.39 (3H, s), 3.71-3.74 (2H, m), 3.79-3.85 (1H, m), 3.86-4.00 (3H, m), 4.06-4.14 (4H, m), 4.23-4.30 (1H, m), 6.40 (1H, dd, J=8.1, 2.4 Hz), 6.48 (1H, d, J=2.4 Hz), 7.02 (1H, d, J=8.4 Hz).

### Reference Example 168

3-[4-(2-Methoxyethoxy)-2-(tetrahydrofuran-2-ylmethoxy)phenyl]propanoate (1.04 g) was dissolved in tetrahydrofuran (24 ml), a 1.5 M diisobutylaluminum hydride solution in toluene (8.0 ml) was added at 0° C., and the mixture was stirred at room temperature for 20 min. Sodium sulfate decahydrate (3.87 g) and diethyl ether (70 ml) were added to the reaction mixture, and the mixture was stirred overnight. The resulting solid was filtered off and the filtrate was concentrated. The obtained residue was subjected to silica gel column chromatography, and eluted with ethyl acetate-hexane (3:7-1:1, v/v) to give 3-[4-(2-methoxyethoxy)-2-(tetrahydrofuran-2-ylmethoxy)phenyl]propan-1-ol as a colorless oil (0.50 g, yield: 54%).

¹H-NMR (300 MHz, CDCl₃) δ:1.66-2.12 (6H, m), 2.58-2.67 (2H, m), 2.72-2.82 (1H, m), 3.44 (3H, s), 3.51 (2H, q, J=5.7 Hz), 3.72-3.75 (2H, m), 3.80-4.00 (4H, m), 4.07-4.11 (2H, m), 4.24-4.32 (1H, m), 6.43-6.48 (2H, m), 7.02 (1H, d, J=8.4 Hz)

### Reference Example 169

To a solution (400 ml) of 2,4-dihydroxybenzaldehyde (30.0 g), 2-methoxyethanol (21.4 g) and triphenylphosphine (74.0 g) in toluene was added dropwise a 40% diethyl azodicarboxylate toluene solution (128 ml) at 0° C. over about 25 min, and the mixture was warmed to room temperature and stirred for 90 min. The reaction mixture was concentrated, and the residue was mixed with ethyl acetate, the insoluble material was filtrated. The filtrate was concentrated, and the residue was subjected to silica gel column chromatography and eluted with ethyl acetate-hexane (1:5-1:2, v/v) to give 2-hydroxy-4-(2-methoxyethoxy)benzaldehyde (14.7 g, yield: 34%) as colorless crystals.

¹H-NMR (300 MHz, CDCl₃) δ:3.45 (3H, s), 3.74-3.78 (2H, m), 4.15-4.18 (2H, m), 6.44 (1H, d, J=2.4 Hz), 6.57 (1H, dd, J=8.7, 2.4 Hz), 7.42 (1H, d, J=8.7 Hz), 9.71 (1H, s), 11.46 (1H, s).

### Reference Example 170

To a solution (200 ml) of 2-hydroxy-4-(2-methoxyethoxy)benzaldehyde (11.4 g) in tetrahydrofuran was added [(ethoxycarbonyl)methylene]triphenyl phosphorane (22.2 g) at 0° C., and the mixture was warmed to room temperature and stirred for 4 hr. The reaction mixture was concentrated, and the residue was subjected to silica gel column chromatography and eluted with ethyl acetate-hexane (2:3, v/v). The obtained crude crystals were recrystallized from ethyl acetate-hexane to give ethyl (2E)-3-[2-hydroxy-4-(2-methoxyethoxy)phenyl]acrylate (15.7 g, yield: 82%) as colorless crystals.

¹H-NMR (300 MHz, CDCl₃) δ:1.33 (3H, t, J=7.2 Hz), 3.47 (3H, s), 3.76-3.78 (2H, m), 4.10-4.13 (2H, m), 4.26 (2H, q, J=7.2 Hz), 6.44-6.52 (3H, m), 7.09 (1H, s), 7.37 (1H, d, J=9.3 Hz), 7.92 (1H, d, J=16.2 Hz).

### Reference Example 171

To a mixed solution of ethyl (2E)-3-[2-hydroxy-4-(2-methoxyethoxy)phenyl]acrylate (2.00 g) in acetonitrile (20 ml) and N,N-dimethylformamide (10 ml) were added bromomethylcyclohexane (4.04 g), potassium carbonate (3.10 g) and sodium iodide (2.37 g), and the mixture was heated under reflux for 40 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:4, v/v) to give ethyl (2E)-3-[2-(cyclohexylmethoxy)-4-(2-methoxyethoxy)phenyl]acrylate (2.22 g, yield: 81%) as colorless crystals.

¹H-NMR (300 MHz, CDCl₃) δ:1.07-1.38 (8H, m), 1.75-1.90 (6H, m), 3.45 (3H, s), 3.75-3.79 (4H, m), 4.12-4.15 (2H, m), 4.24 (2H, q, J=7.2 Hz), 6.40-6.49 (3H, m), 7.43 (1H, d, J=9.3 Hz), 7.93 (1H, d, J=15.9 Hz).

### Reference Example 172

To a mixed solution of ethyl (2E)-3-[2-(cyclohexylmethoxy)-4-(2-methoxyethoxy)phenyl]acrylate (2.21 g) in tetrahydrofuran (10 ml) and ethanol (10 ml) was added a 1N aqueous sodium hydroxide solution (12 ml), and the mixture was stirred at 60° C. for 3 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethyl acetate-hexane to give (2E)-3-[2-(cyclohexylmethoxy)-4-(2-methoxyethoxy)phenyl]acrylic acid (1.91 g, yield: 93%) as colorless crystals. melting point 111.5-112.5° C.

### Reference Example 173

To a solution of ethyl (2E)-3-[2-hydroxy-4-(2-methoxyethoxy)phenyl]acrylate (2.00 g) in N,N-dimethylformamide (10 ml) were added (bromomethyl)cyclopropane (1.57 g) and potassium carbonate (2.07 g), and the mixture was stirred at room temperature for 20 hr. Water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:4, v/v) to give ethyl (2E)-3-[2-(cyclopropylmethoxy)-4-(2-methoxyethoxy)phenyl]acrylate (2.03 g, yield: 84%) as colorless crystals.

¹H-NMR (300 MHz, CDCl₃) δ:0.33-0.38 (2H, m), 0.63-0.69 (2H, m), 1.29-1.36 (4H, m), 3.45 (3H, s), 3.72-3.76 (2H, m), 3.83 (2H, d, J=6.9 Hz), 4.09-4.13 (2H, m), 4.24 (2H, q, J=7.2 Hz), 6.45-6.51 (3H, m), 7.43 (1H, d, J=8.4 Hz), 7.94 (1H, d, J=16.2 Hz).

### Reference Example 174

To a mixed solution of ethyl (2E)-3-[2-(cyclopropylmethoxy)-4-(2-methoxyethoxy)phenyl]acrylate (2.02 g) in tetrahydrofuran (10 ml) and ethanol (10 ml) was added a 1N aqueous sodium hydroxide solution (13 ml), and the mixture was stirred at 60° C. for 2 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethyl acetate-hexane to give (2E)-3-[2-(cyclopropylmethoxy)-4-(2-methoxyethoxy)phenyl]acrylic acid (1.85 g, yield: 99%) as colorless crystals. melting point 117.8-118.8° C.

### Reference Example 175

To a mixed solution of ethyl (2E)-3-[2-hydroxy-4-(2-methoxyethoxy)phenyl]acrylate (2.00 g), 1-Boc-4-hydroxypiperidine (2.30 g) and tributylphosphine (4.58 g) in toluene (260 ml) and tetrahydrofuran (10 ml) was slowly added 1,1′-(azodicarbonyl)dipiperidine (5.28 g) at room temperature, and the mixture was stirred at room temperature for 30 hr. The reaction mixture was concentrated, 1N hydrochloric acid was added to the residue, water was further poured thereinto, and the mixture was extracted with ethyl acetate. The ethyl acetate layer was washed with saturated brine, dried (MgSO₄), and concentrated. An ethyl acetate-hexane (1:1, v/v) mixed solvent was added to the obtained residue, and the insoluble material was filtrated. The filtrate was concentrated, and the residue was subjected to silica gel column chromatography and eluted with ethyl acetate-hexane (2:3, v/v) to give tert-butyl 4-[2-[(1E)-3-ethoxy-3-oxoprop-1-en-1-yl]-5-(2-methoxyethoxy)phenoxy]piperidine-1-carboxylate (2.54 g, yield: 75%) as a colorless oil.

¹H-NMR (300 MHz, CDCl₃) δ:1.32 (3H, t, J=7.2 Hz), 1.47 (9H, s), 1.76-1.86 (2H, m), 1.87-1.99 (2H, m), 3.37-3.50 (5H, m), 3.64-3.76 (4H, m), 4.12-4.14 (2H, m), 4.24 (2H, q, J=7.2 Hz), 4.48-4.52 (1H, m), 6.39 (1H, d, J=16.2 Hz), 6.50-6.52 (2H, m), 7.44-7.47 (1H, m), 7.91 (1H, d, J=16.2 Hz).

### Reference Example 176

To a mixed solution of tert-butyl 4-[2-[(1E)-3-ethoxy-3-oxoprop-x-en-1-yl]-y-(2-methoxyethoxy)phenoxy]piperidine-1-carboxylate (2.54 g) in tetrahydrofuran (10 ml) and ethanol (10 ml) was added a 1N aqueous sodium hydroxide solution (11.3 ml), and the mixture was stirred at 60° C. for 90 min. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated to give (2E)-3-[2-{[1-(tert-butoxycarbonyl)piperidin-4-yl]oxy}-4-(2-methoxyethoxy)phenyl]acrylic acid (2.28 g, yield: 95%) as a pale-yellow amorphous solid.

¹H-NMR (300 MHz, DMSO-d₆) δ:1.40 (9H, s), 1.55-1.61 (2H, m), 1.85-1.95 (2H, m), 3.20-3.31 (5H, m), 3.57-3.65 (4H, m), 4.12-4.15 (2H, m), 4.69-4.76 (1H, m), 6.38 (1H, d, J=16.2 Hz), 6.57 (1H, dd, J=8.4, 2.1 Hz), 6.71 (1H, d, J=2.1 Hz), 7.61 (1H, d, J=8.4 Hz), 7.75 (1H, d, J=16.2 Hz), 12.09 (1H, brs).

### Reference Example 177

To a mixed solution of ethyl (2E)-3-[2-(cyclohexyloxy)-4-(2-methoxyethoxy)phenyl]acrylate (2.07 g) in tetrahydrofuran (10 ml) and ethanol (10 ml) was added a 1N aqueous sodium hydroxide solution (12 ml), and the mixture was stirred at 60° C. for 90 min. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethyl acetate-hexane to give (2E)-3-[2-(cyclohexyloxy)-4-(2-methoxyethoxy)phenyl]acrylic acid (1.59 g, yield: 83%) as colorless crystals. melting point 102.0-113.6° C.

### Reference Example 178

To a solution of ethyl (2E)-3-[2-hydroxy-4-(2-methoxyethoxy)phenyl]acrylate (2.70 g) in N,N-dimethylformamide (22 ml) were added tetrahydrofurfuryl bromide (8.58 g), sodium iodide (7.56 g) and potassium carbonate (6.97 g), and the mixture was stirred at 80° C. for 170 hr. Water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:4, v/v) to give ethyl (2E)-3-[4-(2-methoxyethoxy)-2-(tetrahydrofuran-2-ylmethoxy)phenyl]acrylate (2.65 g, yield: 75%) as a colorless oil.

¹H-NMR (300 MHz, CDCl₃) δ:1.31 (3H, t, J=7.2 Hz), 1.80-2.14 (4H, m), 3.45 (3H, s), 3.73-3.76 (2H, m), 3.82-3.88 (1H, m), 3.90-4.08 (3H, m), 4.12-4.14 (2H, m), 4.23 (2H, q, J=7.2 Hz), 4.30-4.35 (1H, m), 6.42 (1H, d, J=16.2 Hz), 6.50-6.52 (2H, m), 7.42 (1H, d, J=9.0 Hz), 7.91 (1H, d, J=16.2 Hz).

### Reference Example 179

To a mixed solution of ethyl (2E)-3-[4-(2-methoxyethoxy)-2-(tetrahydrofuran-2-ylmethoxy)phenyl]acrylate (2.65 g) in tetrahydrofuran (10 ml) and ethanol (10 ml) was added a 1N aqueous sodium hydroxide solution (15 ml), and the mixture was stirred at room temperature for 1 hr. The reaction mixture was concentrated and neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethyl acetate-hexane to give (2E)-3-[4-(2-methoxyethoxy)-2-(tetrahydrofuran-2-ylmethoxy)phenyl]acrylic acid as colorless crystals (1.99 g, yield: 81%) melting point 106.1-107.7° C.

### Reference Example 180

To a solution of ethyl (2E)-3-[2-hydroxy-4-(2-methoxyethoxy)phenyl]acrylate (1.40 g) in N,N-dimethylformamide (15 ml) were added 2,4-dichlorobenzyl chloride (1.59 g) and potassium carbonate (1.45 g), and the mixture was stirred at room temperature for 60 hr. Water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:4, v/v) to give ethyl (2E)-3-[2-[(2,4-dichlorobenzyl)oxy]-4-(2-methoxyethoxy)phenyl]acrylate (2.07 g, yield: 92%) as colorless crystals. melting point 100.8-101.1° C.

### Reference Example 181

To a mixed solution of ethyl (2E)-3-[2-[(2,4-dichlorobenzyl)oxy]-4-(2-methoxyethoxy)phenyl]acrylate (1.95 g) in tetrahydrofuran (10 ml) and ethanol (10 ml) was added a 1N aqueous sodium hydroxide solution (9 ml), and the mixture was stirred at 60° C. for 90 min. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from diisopropyl ether-methanol to give (2E)-3-[2-[(2,4-dichlorobenzyl)oxy]-4-(2-methoxyethoxy)phenyl]acrylic acid (1.80 g, yield: 99%) as colorless crystals. melting point 175.6-177.5° C.

### Reference Example 182

To a solution of ethyl (2E)-3-[2-hydroxy-4-(2-methoxyethoxy)phenyl]acrylate (900 mg) in N,N-dimethylformamide (15 ml) was added sodium hydride (60% in oil, 188 mg), and the mixture was stirred at room temperature for 1 hr. 2,3,5-Trichloropyridine (925 mg) was added to the reaction mixture, and the mixture was stirred at 80° C. for 36 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:4, v/v). The obtained crude crystals were recrystallized from ethyl acetate-hexane to give ethyl (2E)-3-[2-[(3,5-dichloropyridin-2-yl)oxy]-4-(2-methoxyethoxy)phenyl]acrylate (1.01 g, yield: 72%) as colorless crystals. melting point 70.7-71.3° C.

### Reference Example 183

To a mixed solution of ethyl (2E)-3-[2-[(3,5-dichloropyridin-2-yl)oxy]-4-(2-methoxyethoxy)phenyl]acrylate (1.45 g) in tetrahydrofuran (15 ml) and ethanol (15 ml) was added a 1N aqueous sodium hydroxide solution (7 ml), and the mixture was stirred at 60° C. for 1 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from tetrahydrofuran-hexane to give (2E)-3-[2-[(3,5-dichloropyridin-2-yl)oxy]-4-(2-methoxyethoxy)phenyl]acrylic acid (1.24 g, yield: 92%) as colorless crystals. melting point 182.3-184.0° C.

### Reference Example 184

To a solution of ethyl (2E)-3-[2-hydroxy-4-(2-methoxyethoxy)phenyl]acrylate (1.20 g) in N,N-dimethylformamide (15 ml) were added 4-(chloromethyl)-5-methyl-2-phenyl-1,3-oxazole (1.03 g) and potassium carbonate (1.24 g), and the mixture was stirred at room temperature for 40 hr. Water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (2:3, v/v). The obtained crude crystals were recrystallized from ethyl acetate-hexane to give ethyl (2E)-3-{4-(2-methoxyethoxy)-2-[(5-methyl-2-phenyl-1,3-oxazol-4-yl)methoxy]phenyl}acrylate (1.85 g, yield: 94%) as colorless crystals. melting point 109.4-110.0° C.

### Reference Example 185

To a mixed solution of ethyl (2E)-3-{4-(2-methoxyethoxy)-2-[(5-methyl-2-phenyl-1,3-oxazol-4-yl)methoxy]phenyl}acrylate (1.73 g) in tetrahydrofuran (15 ml) and ethanol (15 ml) was added a 1N aqueous sodium hydroxide solution (12 ml), and the mixture was stirred at 60° C. for 6 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from tetrahydrofuran-hexane to give (2E)-3-{4-(2-methoxyethoxy)-2-[(5-methyl-2-phenyl-1,3-oxazol-4-yl)methoxy]phenyl}acrylic acid (1.56 g, yield: 96%) as colorless crystals. melting point 194.3-195.8° C.

### Reference Example 186

To a mixed solution of ethyl (2E)-3-{4-(2-methoxyethoxy)-2-[2-nitro-5-(trifluoromethyl)phenoxy]phenyl}acrylate (1.01 g) in tetrahydrofuran (5 ml) and ethanol (5 ml) was added a 1N aqueous sodium hydroxide solution (5 ml), and the mixture was stirred at 70° C. for 2 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethanol-hexane to give (2E)-3-{4-(2-methoxyethoxy)-2-[2-nitro-5-(trifluoromethyl)phenoxy]phenyl}acrylic acid (0.58 g, yield: 61%) as colorless crystals.

¹H-NMR (300 MHz, DMSO-d₆) δ:3.25 (3H, s), 3.57-3.62 (2H, m), 4.04-4.11 (2H, m), 6.38-6.92 (3H, m), 7.46-7.90 (4H, m), 8.32-8.35 (1H, m), 12.09 (1H, brs).

### Reference Example 187

To a solution of ethyl (2E)-3-[2-hydroxy-4-(2-methoxyethoxy)phenyl]acrylate (1.19 g) in N,N-dimethylformamide (8 ml) were added 3-chloro-4-fluorobenzotrifluoride (1.06 g) and potassium carbonate (1.23 g), and the mixture was stirred at 80° C. for 55 hr. After cooling, water was poured into the reaction mixture, and the mixture was extracted with ethyl acetate. The ethyl acetate layer was washed with saturated brine, dried (MgSO₄) and concentrated. The obtained residue was subjected to silica gel column chromatography, and eluted with ethyl acetate-hexane (1:3, v/v) to give ethyl (2E)-3-[2-[2-chloro-4-(trifluoromethyl)phenoxy]-4-(2-methoxyethoxy)phenyl]acrylate (1.76 g, yield: 88%) as colorless crystals.

¹H-NMR (300 MHz, CDCl₃) δ:1.30 (3H, t, J=7.2 Hz), 3.41 (3H, s), 3.69-3.72 (2H, m), 4.05-4.09 (2H, m), 4.22 (2H, q, J=7.2 Hz), 6.36 (1H, d, J=2.4 Hz), 6.43 (1H, d, J=16.2 Hz), 6.77 (1H, dd, J 8.7, 2.4 Hz), 6.96 (1H, d, J=8.7 Hz), 7.45 (1H, d, J=8.7 Hz), 7.59 (1H, d, J=8.7 Hz), 7.75 (1H, s), 7.85 (1H, d, J=16.2 Hz).

### Reference Example 188

To a mixed solution of ethyl (2E)-3-[2-[2-chloro-4-(trifluoromethyl)phenoxy]-4-(2-methoxyethoxy)phenyl]acrylate (1.76 g) in tetrahydrofuran (8 ml) and ethanol (8 ml) was added a 1N aqueous sodium hydroxide solution (8 ml), and the mixture was stirred at 50° C. for 90 min. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethyl acetate-hexane to give (2E)-3-[2-[2-chloro-4-(trifluoromethyl)phenoxy]-4-(2-methoxyethoxy)phenyl]acrylic acid (1.60 g, yield: 97%) as colorless crystals. melting point 165.6-166.0° C.

### Reference Example 189

To a solution of ethyl (2E)-3-(4-(2-methoxyethoxy)-2-{[(trifluoromethyl)sulfonyl]oxy}phenyl)acrylate (1.50 g) in tetrahydrofuran (20 ml) were added 4-aminobenzotrifluoride (948 mg), racemic-2,2′-bis(diphenylphosphino)-1,1′-binaphthyl (282 mg), palladium acetate (67 mg) and cesium carbonate (1.83 g), and the mixture was heated under reflux for 16 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:4, v/v) to give ethyl (2E)-3-(4-(2-methoxyethoxy)-2-{[4-(trifluoromethyl)phenyl]amino}phenyl)acrylate (1.00 g, yield: 65%) as pale-yellow crystals.

¹H-NMR (300 MHz, CDCl₃) δ:1.30 (3H, t, J=7.1 Hz), 3.43 (3H, s), 3.73-3.77 (2H, m), 4.07-4.10 (2H, m), 4.23 (2H, q, J=7.1 Hz), 5.85 (1H, s), 6.31 (1H, d, J=15.7 Hz), 6.69-6.73 (1H, m), 6.88 (1H, d, J=2.4 Hz), 6.98 (2H, d, J=8.7 Hz), 7.47-7.56 (3H, m), 7.82 (1H, d, J=15.7 Hz).

### Reference Example 190

To a mixed solution of ethyl (2E)-3-(4-(2-methoxyethoxy)-2-{[4-(trifluoromethyl)phenyl]amino}phenyl)acrylate (1.42 g) in tetrahydrofuran (8 ml) and ethanol (8 ml) was added a 1N aqueous sodium hydroxide solution (7 ml), and the mixture was stirred at 50° C. for 2 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethanol-hexane to give (2E)-3-(4-(2-methoxyethoxy)-2-{[4-(trifluoromethyl)phenyl]amino}phenyl)acrylic acid (0.96 g, yield: 72%) as yellow crystals.

¹H-NMR (300 MHz, DMSO-d₆) δ:3.29 (3H, s), 3.62-3.65 (2H, m), 4.05-4.16 (2H, m), 6.33-6.43 (2H, m), 6.77-6.98 (3 H, m), 7.48-7.80 (4H, m), 8.60 (1H, s), 12.12 (1H, s)

### Reference Example 191

To a solution of ethyl (2E)-3-[2-hydroxy-4-(2-methoxyethoxy)phenyl]acrylate (1.48 g) in N,N-dimethylformamide (10 ml) were added 2,5-dibromothiazole (1.61 g) and potassium carbonate (1.53 g), and the mixture was stirred at 80° C. for 22 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:2, v/v) to give ethyl (2E)-3-[2-[(5-bromo-1,3-thiazol-2-yl)oxy]-4-(2-methoxyethoxy)phenyl]acrylate (1.69 g, yield: 71%) as a colorless oil.

¹H-NMR (300 MHz, CDCl₃) δ:1.31 (3H, t, J=7.2 Hz), 3.43 (3H, s), 3.73-3.75 (2H, m), 4.10°-4.14 (2H, m), 4.23 (2H, q, J=7.2 Hz), 6.37 (1H, d, J=16.1 Hz), 6.83 (1H, s), 6.87 (1H, d, J=8.7 Hz), 7.16 (1H, s), 7.60 (1H, d, J=8.7 Hz), 7.79 (1H, d, J=16.1 Hz).

### Reference Example 192

To a mixed solution of ethyl (2E)-3-[2-[(5-bromo-1,3-thiazol-2-yl)oxy]-4-(2-methoxyethoxy)phenyl]acrylate (1.67 g) in tetrahydrofuran (10 ml) and ethanol (10 ml) was added a 1N aqueous sodium hydroxide solution (8 ml), and the mixture was stirred at 50° C. for 30 min. After cooling, the reaction mixture was concentrated, and the concentrate was neutralized with 1N hydrochloric acid. Water was poured into the obtained mixture, and the mixture was extracted with ethyl acetate. The ethyl acetate layer, was washed with saturated brine, dried (MgSO₄), and concentrated. The obtained residue was recrystallized from ethanol-hexane to give (2E)-3-[2-[(5-bromo-1,3-thiazol-2-yl)oxy]-4-(2-methoxyethoxy)phenyl]acrylic acid (1.46 g, yield: 93%) as colorless crystals. melting point 144.0-145.1° C.

### Reference Example 193

To a solution of ethyl (2E)-3-(4-7(2-methoxyethoxy)-2-{[(trifluoromethyl)sulfonyl]oxy}phenyl)acrylate (1.66 g) in tetrahydrofuran (20 ml) were added 2-chloro-4-(trifluoromethyl)aniline (1.89 g), racemic-2,2′-bis(diphenylphosphino)-1,1′-binaphthyl (519 mg), palladium acetate (140 mg) and cesium carbonate (2.06 g), and the mixture was heated under reflux for 8 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:3, v/v) to give ethyl (2E)-3-[2-{[2-chloro-4-(trifluoromethyl)phenyl]amino}-4-(2-methoxyethoxy)phenyl]acrylate (1.00 g, yield: 54%) as yellow crystals.

¹H-NMR (300 MHz, CDCl₃) δ:1.29 (3H, t, J=7.2 Hz), 3.44 (3H, s), 3.73-3.79 (2H, m), 4.16-4.27 (4H, m), 6.23 (1H, s), 6.34 (1H, d, J=15.9 z), 6.55-6.59 (1H, m), 6.85-6.91 (2H, m), 7.32-7.62 (3H, m), 7.77 (1H, d, J=15.9 Hz).

### Reference Example 194

To a mixed solution of ethyl (2E)-3-[2-{[2-chloro-4-(trifluoromethyl)phenyl]amino}-4-(2-methoxyethoxy)phenyl]acrylate (1.00 g) in tetrahydrofuran (5 ml) and ethanol (5 ml) was added a 1N aqueous sodium hydroxide solution (4.5 ml), and the mixture was stirred at 60° C. for 30 min. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethanol-hexane to give (2E)-3-[2-{[2-chloro-4-(trifluoromethyl)phenyl]amino}-4-(2-methoxyethoxy)phenyl]acrylic acid (515 mg, yield: 55%) as colorless crystals.

¹H-NMR (300 MHz, DMSO-d₆) δ:3.29 (3H, s), 3.62-3.65 (2H, m), 4.10-4.13 (2H, m), 6.39 (1H, t, J=16.1 Hz), 6.53 (1H, d, J=9.0 Hz), 6.80 (1H, d, J=2.4 Hz), 6.89-6.93 (1 H, m), 7.38-7.42 (1H, m), 7.54 (1H, t, J=16.1 Hz), 7.74 (1H, d, J=2.1 Hz), 7.86 (1H, d, J=9.0 Hz), 8.18 (1H, s), 12.19 (1H, s).

### Reference Example 195

To a solution of ethyl (2E)-3-[2-hydroxy-4-(2-methoxyethoxy)phenyl]acrylate (1.10 g) in N,N-dimethylformamide (10 ml) were added 2-chloro-5-nitro-3-picoline (1.03 g) and potassium carbonate (1.14 g), and the mixture was stirred at 80° C. for 2 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:1, v/v) to give ethyl (2E)-3-{4-(2-methoxyethoxy)-2-[(3-methyl-5-nitropyridin-2-yl)oxy]phenyl}acrylate (1.63 g, yield: 98%) as pale-yellow crystals.

¹H-NMR (300 MHz, CDCl₃) δ:1.27 (3H, t, J=7.1 Hz), 2.52 (3H, s), 3.44 (3H, s), 3.73-3.76 (2H, m), 4.12-4.15 (2H, m), 4.18 (2H, q, J=7.1 Hz), 6.32 (1H, d, J=16.2 Hz), 6.68 (1 H, d, J=2.4 Hz), 6.90 (1H, dd, J=8.7, 2.4 Hz), 7.63 (1H, d, J=8.7 Hz), 7.64 (1H, d, J=16.2 Hz), 8.35 (1H, d, J=1.8 Hz), 8.81 (1H, d, J=1.8 Hz).

### Reference Example 196

To a solution of ethyl (2E)-3-[2-hydroxy-4-(2-methoxyethoxy)phenyl]acrylate (1.05 g) in N,N-dimethylformamide (10 ml) were added 3-chloro-6-(trifluoromethyl)pyridazine (1.48 g) and potassium carbonate (1.53 g), and the mixture was stirred at 80° C. for 3 hr. After cooling, water was poured into the reaction mixture, and the mixture was extracted with ethyl acetate. The ethyl acetate layer was washed with saturated brine, dried (MgSO₄), and concentrated. The obtained residue was, subjected to silica gel column chromatography, and eluted with ethyl acetate-methanol (49:1, v/v). The obtained crude crystals were recrystallized from ethyl acetate-hexane to give ethyl (2E)-3-(4-(2-methoxyethoxy)-2-{[6-(trifluoromethyl)pyridazin-3-yl]oxy}phenyl)acrylate (2.14 g, yield: 90%) as colorless crystals.

¹H-NMR (300 MHz, CDCl₃) δ:1.27 (3H, t, J=7.1 Hz), 3.43 (3H, s), 3.73-3.75 (2H, m), 4.11-4.14 (2H, m), 4.19 (2H, q, J=7.1 Hz), 6.35 (1H, d, J=16.2 Hz), 6.75 (1H, d, J=2.7 Hz), 6.91 (1H, dd, J 8.7, 2.7 Hz), 7.40 (1H, d, J=9.0 Hz), 7.64 (1H, d, J=8.7 Hz), 7.70 (1H, d, J=16.2 Hz), 7.84 (1H, d, J=9.0 Hz).

### Reference Example 197

To a solution of 2-[(3,5-dichloropyridin-2-yl)oxy]-4-(2-methoxyethoxy)benzaldehyde (500 mg), in acetic acid (8 ml) were added methylmalonic acid (859 mg) and pyrrolidine (827 mg), and the mixture was stirred at 100° C. for 24 hr. 1N Hydrochloric acid (1 ml) and water (10 ml) were added to the reaction mixture, and the mixture was stirred at room temperature for 20 min. Water was poured into the obtained mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:3, v/v). The obtained crude crystals were recrystallized from ethyl acetate-hexane to give (2E)-3-[2-[(3,5-dichloropyridin-2-yl)oxy]-4-(2-methoxyethoxy)phenyl]-2-methylacrylic acid (455 mg, yield: 78%) as colorless crystals melting point 126.0-127.0° C.

### Reference Example 198

To a solution of triethyl 2-phosphonobutyrate (0.83 g) in tetrahydrofuran (10 ml) was added sodium hydride (60% in oil, 145 mg) at 0° C., and the mixture was stirred at 0° C. for 15 min. To this reaction mixture was added a solution of 2-[(3,5-dichloropyridin-2-yl)oxy]-4-(2-methoxyethoxy)benzaldehyde (1.02 g) in tetrahydrofuran (10 ml) at 0° C., and the mixture was warmed to room temperature and stirred for 3 hr. Water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:4, v/v) to give ethyl (2E)-2-[2-[(3,5-dichloropyridin-2-yl)oxy]-4-(2-methoxyethoxy)benzylidene]butanoate (922 mg, yield: 70%) as a colorless amorphous solid.

¹H-NMR (300 MHz, CDCl₃) δ:1.08 (3H, t, J=7.2 Hz), 1.26 (3H, t, J=7.2 Hz), 2.45 (2H, q, J=7.2 Hz), 3.44 (3H, s), 3.73-3.76 (2H, m), 4.11-4.14 (2H, m), 4.18 (2H, q, J=7.2 Hz), 6.74 (1H, d, J=3.0 Hz), 6.86 (1H, dd, J=9.0, 3.0 Hz), 7.34 (1H, d, J=9.0 Hz), 7.48 (1H, s), 7.75 (1H, d, J=2.1 Hz), 7.91 (1H, d, J=2.1 Hz).

### Reference Example 199

To a mixed solution of ethyl (2E)-2-[2-[(3,5-dichloropyridin-2-yl)oxy]-4-(2-methoxyethoxy)benzylidene]butanoate (908 mg) in tetrahydrofuran (5 ml) and ethanol (5 ml) was added a 1N aqueous sodium hydroxide solution (6 ml), and the mixture was stirred at 60° C. for 6 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethanol-hexane to give (2E)-2-[2-[(3,5-dichloropyridin-2-yl)oxy]-4-(2-methoxyethoxy)benzylidene]butanoic acid (772 mg, yield: 91%) as colorless crystals. melting point 139.0-139.9° C.

### Reference Example 200

To a solution of 4-iodo-3-nitrophenol (5.33 g) in acetone (60 ml) were added 2-bromoethyl methyl ether (11.1 g), sodium iodide (3.65 g) and potassium carbonate (6.98 g), and the mixture was heated under reflux for 20 hr. The reaction mixture was concentrated, water was poured into the obtained residue, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:3, v/v) to give 1-iodo-4-(2-methoxyethoxy)-2-nitrobenzene (5.59 g, yield: 86%) as pale-yellow crystals.

¹H-NMR (300 MHz, CDCl₃) δ:3.44 (3H, s), 3.76 (2H, t, J=4.5 Hz), 4.15 (2H, t, J=4.5 Hz), 6.90 (1H, dd, J=9.0, 2.7 Hz), 7.46 (1H, d, J=2.7 Hz), 7.87 (1H, d, J=9.0 Hz).

### Reference Example 201

To a solution of 1-iodo-4-(2-methoxyethoxy)-2-nitrobenzene (5.59 g) in acetonitrile (50 ml) were added ethyl acrylate (2.62 g), triethylamine (3.62 g) and palladium acetate (116 mg), and the mixture was heated under reflux for 5 hr. After cooling, the reaction mixture was concentrated, water was poured into the obtained residue, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:1, v/v) to give ethyl (2E)-3-[4-(2-methoxyethoxy)-2-nitrophenyl]acrylate (4.94 gi yield: 96%) as pale-yellow crystals.

¹H-NMR (300 MHz, CDCl₃) δ:1.33 (3H, t, J=7.2 Hz), 3.46 (3H, s), 3.78 (2H, t, J=4.5 Hz), 4.21 (2H, t, J=4.5 Hz), 4.27 (2H, q, J=7.2 Hz), 6.30 (1H, d, J=15.9 Hz), 7.21 (1H, dd, J=8.7, 2.7 Hz), 7.53 (1H, d, J=2.7 Hz), 7.57 (1H, d, J=8.7 Hz), 8.03 (1H, d, J=15.9 Hz).

### Reference Example 202

To a mixture of water (60 ml) and zinc (7.56 g) was added a solution of (2E)-3-[4-(2-methoxyethoxy)-2-nitrophenyl]acrylate (6.83 g) in acetic acid (60 ml), and the mixture was stirred at room temperature for 30 min. The reaction mixture was concentrated, the obtained residue was basified with a 1N aqueous sodium hydroxide solution, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:1, v/v) to give ethyl (2E)-3-[2-amino-4-(2-methoxyethoxy)phenyl]acrylate (5.03 g, yield: 82%) as pale-yellow crystals.

¹H-NMR (300 MHz, CDCl₃) δ:1.32 (3H, t, J=7.2 Hz), 3.44 (3H, s), 3.73 (2H, t, J=4.8 Hz), 3.95 (2H, s), 4.09 (2H, t, J=4.8 Hz), 4.24 (2H, q, J=7.2 Hz), 6.20-6.25 (2H, m), 6.36 (1H, m), 7.33 (1H, d, J=8.7 Hz), 7.74 (1H, d, J=15.6 Hz).

### Reference Example 203

To a solution of ethyl (2E)-3-[2-amino-4-(2-methoxyethoxy)phenyl]acrylate (640 mg) in tetrahydrofuran (10 ml) were added 2,4-dichlorobenzoyl chloride (685 mg) and triethylamine (490 mg), and the mixture was stirred at room temperature for 1 hr. The reaction mixture was concentrated, water was added to the obtained residue, and the mixture was extracted with ethyl acetate. The ethyl acetate layer was washed with an aqueous sodium hydrogencarbonate solution and saturated brine, dried (MgSO₄), and concentrated. The obtained crude crystals were recrystallized from ethanol-hexane to give ethyl (2E)-3-[2-[(2,4-dichlorobenzoyl)amino]-4-(2-methoxyethoxy)phenyl]acrylate (812 mg, yield: 77%) as colorless crystals. melting point 170.1-170.5° C.

### Reference Example 204

To a mixed solution of ethyl (2E)-3-[2-[(2,4-dichlorobenzoyl)amino]-4-(2-methoxyethoxy)phenyl]acrylate (472 mg) in tetrahydrofuran (8 ml) and ethanol (8 ml) was added a 1N aqueous sodium hydroxide solution (2.5 ml), and the mixture was stirred at 60° C. for 2 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained crude crystals were recrystallized from methanol-diisopropyl ether to give (2E)-3-[2-[(2,4-dichlorobenzoyl)amino]-4-(2-methoxyethoxy)phenyl]acrylic acid (422 mg, yield: 96%) as colorless crystals. melting point 242.8-244.8° C.

### Reference Example 205

To a solution of 2-hydroxy-4-(methoxymethoxy)benzaldehyde (7.04 g) in N,N-dimethylformamide (50 ml) was added sodium hydride (60% in oil, 1.88 g), and the mixture was stirred at room temperature for 30 min. 2,3,5-Trichloropyridine (7.40 g) were added to the reaction mixture, and the mixture was stirred at 110° C. for 14 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:5, v/v) to give 2-[(3,5-dichloropyridin-2-yl)oxy]-4-(methoxymethoxy)benzaldehyde (5.33 g, yield: 42%) as colorless crystals.

¹H-NMR (300 MHz, CDCl₃) δ:3.48 (3H, s), 5.23 (2H, s), 6.85 (1H, d, J=2.4 Hz), 7.03 (1H, dd, J=9.0, 2.4 Hz), 7.82 (1H, d, J=2.4 Hz), 7.92 (1H, d, J=9.0 Hz), 7.95 (1H, d, J=2.4 Hz), 10.05 (1H, s).

### Reference Example 206

To a solution of 2-[(3,5-dichloropyridin-2-yl)oxy]-4-(methoxymethoxy)benzaldehyde (4.07 g) in acetone (25 ml) was added 1N hydrochloric acid (25 ml), and the mixture was heated under reflux for 2 hr. After cooling, the reaction mixture was concentrated, and the concentrate was neutralized with a 1N aqueous sodium hydroxide solution. Water was poured into the obtained mixture, and the mixture was extracted with ethyl acetate. After cooling, water was poured into the reaction mixture, and the mixture was extracted with ethyl acetate. The ethyl acetate layer was washed with saturated brine, dried (MgSO₄), and concentrated to give 2-[(3,5-dichloropyridin-2-yl)oxy]-4-hydroxybenzaldehyde as colorless crystals (3.60 g, yield: 99%).

¹H-NMR (300 MHz, DMSO-d₆) δ:6.63 (1H, d, J=2.4 Hz), 6.85 (1H, dd, J=8.4, 2.1 Hz), 7.78 (1H, d, J=8.4 Hz), 8.13 (1H, d, J=2.4 Hz), 8.37 (1H, d, J=2.4 Hz), 9.81 (1H, s), 10.95 (1H, s).

### Reference Example 207

To a solution of 2-[(3,5-dichloropyridin-2-yl)oxy]-4-hydroxybenzaldehyde (3.60 g) in N,N-dimethylformamide (25 ml) were added 1-bromo-3-methoxypropane (2.89 g), sodium iodide (2.85 g) and potassium carbonate (3.48 g), and the mixture was stirred at 80° C. for 2 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:4, v/v) to give 2-[(3,5-dichloropyridin-2-yl)oxy]-4-(3-methoxypropoxy)benzaldehyde (3.94 g, yield: 87%) as colorless crystals.

¹H-NMR (300 MHz, CDCl₃) δ:2.01-2.10 (2H, m), 3.34 (3H, s), 3.53 (2H, t, J=6.0 Hz), 4.11 (2H, t, J=6.0 Hz), 6.68 (1H, d, J=2.1 Hz), 6.89 (1H, dd, J=8.7, 2.1 Hz), 7.82 (1H, d, J=2.4 Hz), 7.90 (1H, d, J=8.7 Hz), 7.95 (1H, d, J=2.4 Hz), 10.03 (1H, s).

### Reference Example 208

To a solution of 2-[(3,5-dichloropyridin-2-yl)oxy]-4-(3-methoxypropoxy)benzaldehyde (745 mg) in acetic acid (12 ml) were added methylmalonic acid (1.24 g) and pyrrolidine (1.18 g), and the mixture was stirred at 100° C. for 66 hr. After cooling, 1N hydrochloric acid (2 ml) and water (10 ml) were added to the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:4, v/v). The obtained crude crystals were recrystallized from ethyl acetate-hexane to give (2E)-3-[2-[(3,5-dichloropyridin-2-yl)oxy]-4-(3-methoxypropoxy)phenyl]-2-methylacrylic acid (658 mg, yield: 76%) as colorless crystals. melting point 128.4-129.5° C.

### Reference Example 209

To a solution of (2E)-3-[2-amino-4-(2-methoxyethoxy)phenyl]acrylate (615 mg) in tetrahydrofuran (15 ml) were added 2,4-difluorobenzoyl chloride (490 mg) and triethylamine (480 mg), and the mixture was stirred at room temperature for 2 hr. The reaction mixture was concentrated, an aqueous sodium hydrogencarbonate solution was added to the obtained residue, and the mixture was extracted with ethyl acetate. The ethyl acetate layer was washed with an aqueous sodium hydrogencarbonate solution and saturated brine, dried (MgSO₄), and concentrated. The obtained crude crystals were recrystallized from tetrahydrofuran-hexane to give ethyl (2E)-3-[2-[(2,4-difluorobenzoyl)amino]-4-(2-methoxyethoxy)phenyl]acrylate (718 mg, yield: 76%) as colorless crystals.

¹H-NMR (300 MHz, CDCl₃) δ:1.33 (3H, t, J=7.2 Hz), 3.45 (3H, s), 3.75-3.78 (2H, m), 4.17-4.21 (2H, m), 4.26 (2H, q, J=7.2 Hz), 6.34 (1H, d, J=15.6 Hz), 6.84 (1H, dd, J 8.7, 2.7 Hz), 6.93-7.11 (2H, m), 7.54 (1H, d, J=8.7 Hz), 7.72 (1H, d, J=2.7 Hz), 7.85 (1H, d, J=15.6 Hz), 8.20-8.25 (1H, m), 8.44-8.49 (1H, m).

### Reference Example 210

To a mixed solution of ethyl (2E)-3-[2-[(2,4-difluorobenzoyl)amino]-4-(2-methoxyethoxy)phenyl]acrylate (572 mg) in tetrahydrofuran (6 ml) and ethanol (6 ml) was added a 1N aqueous sodium hydroxide solution (3 ml), and the mixture was stirred at 60° C. for 2 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from tetrahydrofuran-hexane to give (2E)-3-[2-[(2,4-difluorobenzoyl)amino]-4-(2-methoxyethoxy)phenyl]acrylic acid (460 mg, yield: 86%) as colorless crystals.

¹H-NMR (300 MHz, DMSO-d₆) δ:3.31 (3H, s), 3.65-3.68 (2H, m), 4.12-4.15 (2H, m), 6.37 (1H, d, J=15.6 Hz), 6.91 (1H, dd, J=8.7, 2.4 Hz), 7.08 (1H, s), 7.22-7.30 (1H, m), 7.41-7.49 (1H, m), 7.72 (1H, d, J=15.6 Hz), 7.79-7.82 (2H, m), 10.27 (1H, s), 12.22 (1H, s).

### Reference Example 211

To a mixture of water (5 ml) and zinc (379 mg) was added a solution of ethyl (2E)-3-{4-(2-methoxyethoxy)-2-[(3-methyl-5-nitropyridin-2-yl)oxy]phenyl}acrylate (455 mg) in acetic acid (5 ml), and the mixture was stirred at room temperature for 2 hr. The reaction mixture was concentrated, the obtained residue was basified with a 8N aqueous sodium hydroxide solution, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:1, v/v) to give ethyl (2E)-3-[2-[(5-amino-3-methylpyridin-2-yl)oxy]-4-(2-methoxyethoxy)phenyl]acrylate (307 mg, yield: 73%) as brown crystals.

¹H-NMR (300 MHz, CDCl₃) δ:1.30 (3H, t, J=7.2 Hz), 2.23 (3H, s), 3.41 (3H, s), 3.51 (2H, s), 3.67-3.71 (2H, m), 4.03-4.07 (2H, m), 4.21 (2H, q, J=7.2 Hz), 6.38 (1H, s), 6.41 (1H, d, J=15.9 Hz), 6.67 (1H, dd, J=8.7, 2.4 Hz), 6.96 (1H, d, J=2.4 Hz), 7.52-7.55 (2H, m), 7.93 (1H, d, J=15.9 Hz).

### Reference Example 212

To a solution of ethyl (2E)-3-[2-[(5-amino-3-methylpyridin-2-yl)oxy]-4-(2-methoxyethoxy)phenyl]acrylate (300 mg) in tetrahydrofuran (10 ml) was added di-tert-butyl dicarbonate (880 mg), and the mixture was stirred at 60° C. for 11 hr. The reaction mixture was concentrated, the obtained residue was subjected to silica gel column chromatography, and eluted with ethyl acetate-hexane (1:2, v/v) to give ethyl (2E)-3-[2-({5-[(tert-butoxycarbonyl)amino]-3-methylpyridin-2-yl}oxy)-4-(2-methoxyethoxy)phenyl]acrylate (351 mg, yield: 92%) as colorless crystals.

¹H-NMR (300 MHz, CDCl₃) δ:1.29 (3H, t, J=7.2 Hz), 1.52 (9H, s), 2.35 (3H, s), 3.41 (3H, s), 3.69-3.72 (2H, m), 4.05-4.08 (2H, m), 4.20 (2H, q, J=7.2 Hz), 6.36 (1H, d, J=15.9 Hz), 6.34-6.40 (1H, m), 6.49 (1H, d, J=2.7 Hz), 6.74 (1H, dd, J=8.7, 2.7 Hz), 7.57 (1H, d, J=8.7 Hz), 7.78-7.79 (1H, m), 7.85 (1H, d, J=15.9 Hz), 7.93 (1H, s).

### Reference Example 213

To a mixed solution of ethyl (2E)-3-[2-({5-[(tert-butoxycarbonyl)amino]-3-methylpyridin-2-yl}oxy)-4-(2-methoxyethoxy)phenyl]acrylate (351 mg) in tetrahydrofuran (3 ml) and ethanol (3 ml) was added a 1N aqueous sodium hydroxide solution (1.5 ml), and the mixture was stirred at 60° C. for 1 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethanol-hexane to give (2E)-3-[2-({5-[(tert-butoxycarbonyl)amino]-3-methylpyridin-2-yl}oxy)-4-(2-methoxyethoxy)phenyl]acrylic acid (327 mg, yield: 99%) as colorless crystals. melting point 159.0-160.0° C.

### Reference Example 214

To a mixture of ethylene glycol (34 ml) and pyridine (34 ml) was added dropwise chloro(triisopropyl)silane (10.7 ml) at room temperature over 20 min, and the mixture was stirred for 12 hr. Water was poured into the reaction mixture, and the mixture was extracted with ethyl acetate. The aqueous layer was washed with ethyl acetate, and the obtained organic layer was washed with water and saturated brine, dried, and concentrated. The obtained residue was subjected to column chromatography, and eluted with ethyl acetate-hexane (1:20-1:5, v/v) to give 2-[(triisopropylsilyl)oxy]ethanol (10.48 g, yield: 96%) as a colorless oil.

¹H-NMR (300 MHz, CDCl₃) δ:0.99-1.17 (21H, m), 2.17 (1H, t, J=6.2 Hz), 3.58-3.71 (2H, m), 3.74-3.85 (2H, m).

### Reference Example 215

To a mixture of ethyl (2E)-3-(2-{[3-chloro-5-(trifluoromethyl)pyridin-2-yl]oxy}-4-hydroxyphenyl)acrylate (1.50 g), 2-[(triisopropylsilyl)oxy]ethanol (1.01 g), tributylphosphine (2.41 ml) and tetrahydrofuran (50 ml) was added 1,1′-(azodicarbonyl)dipiperidine (1.95 g) at room temperature, and the mixture was stirred for 16 hr. The reaction mixture was concentrated, diisopropyl ether was added, and the precipitated crystals were filtered off. The filtrate was concentrated, and the obtained residue was subjected to silica gel column chromatography and eluted with ethyl acetate-hexane (1:20-1:3, v/v) to give ethyl (2E)-3-(2-{[3-chloro-5-(trifluoromethyl)pyridin-2-yl]oxy}-4-{2-[(triisopropylsilyl)oxy]ethoxy}phenyl)acrylate as a pale-yellow oil (2.02 g, yield: 89%).

¹H-NMR (300 MHz, CDCl₃) δ:0.97-1.15 (21H, m), 1.28 (3H, t, J=7.1 Hz), 3.98-4.12 (4H, m), 4.20 (2H, q, J=7.2 Hz), 6.36 (1H, d, J=16.2 Hz), 6.70 (1H, d, J=2.5 Hz), 6.89 (1H, dd, J=8.8, 2.5 Hz), 7.62 (1H, d, J=8.7 Hz), 7.68 (1H, d, J=16.2 Hz), 8.01 (1H, d, J=2.1 Hz), 8.25 (1H, dd, J=2.1, 0.9 Hz).

### Reference Example 216

A mixture of ethyl (2E)-3-(2-{[3-chloro-5-(trifluoromethyl)pyridin-2-yl]oxy}-4-{2-[(triisopropylsilyl)oxy]ethoxy}phenyl)acrylate (1.22 g), a 1N aqueous sodium hydroxide solution (4.14 ml), tetrahydrofuran (3.2 ml) and ethanol (3.2 ml) was stirred at 50° C. for 2 hr. The reaction mixture was concentrated, 1N hydrochloric acid (4.14 ml) was added, and the mixture was extracted with ethyl acetate. The ethyl acetate layer was washed with saturated brine, dried (MgSO₄) and concentrated. The residue was washed with diethyl ether-hexane, and the insoluble material was filtered off. The filtrate was concentrated. The residue was subjected to silica gel column chromatography, and eluted with ethyl acetate-hexane (1:20-2:1, v/v) to give (2E)-3-(2-{[3-chloro-5-(trifluoromethyl)pyridin-2-yl]oxy}-4-{2-[(triisopropylsilyl)oxy]ethoxy}phenyl)acrylic acid (0.99 g, yield: 85%) as a white solid.

¹H-NMR (300 MHz, CDCl₃) δ:0.95-1.17 (21H, m), 3.88-4.20 (4H, m), 6.36 (1H, d, J=16.0 Hz), 6.70 (1H, d, J=2.5 Hz), 6.90 (1H, dd, J=8.7, 2.5 Hz), 7.64 (1H, d, J=8.9 Hz), 7.76 (1H, d, J=16.0 Hz), 8.02 (1H, dd, J=2.3, 0.4 Hz), 8.25 (1H, dd, J=2.3, 0.9 Hz).

### Reference Example 217

To a mixture of ethyl (2E)-3-(2-{[3-chloro-5-(trifluoromethyl)pyridin-2-yl]oxy}-4-hydroxyphenyl)acrylate (3.00 g), 1,3-diethoxypropan-2-ol (1.38 g), tributylphosphine (4.81 ml) and tetrahydrofuran (100 ml) was added 1,1′-(azodicarbonyl)dipiperidine (3.91 g) at room temperature, and the mixture was stirred for 16 hr. The reaction mixture was concentrated, diisopropyl ether was added, and the precipitated crystals were filtered off. The filtrate was concentrated, and the obtained residue was subjected to silica gel column chromatography and eluted with ethyl acetate-hexane (1:20-1:3, v/v) to give ethyl (2E)-3-{2-{[3-chloro-5-(trifluoromethyl)pyridin-2-yl]oxy}-4-[2-ethoxy-1-(ethoxymethyl)ethoxy]phenyl}acrylate as a colorless oil (1.97 g, yield: 49%). Crystallization from diethyl ether-hexane gave a white powder melting point 66-67.5° C.

¹H-NMR (300 MHz, CDCl₃) δ:1.17 (6H, t, J=7.0 Hz), 1.28 (3H, t, J=7.2 Hz), 3.52 (4H, qd, J=7.0, 0.9 Hz), 3.59-3.74 (4H, m), 4.20 (2H, q, J=7.0 Hz), 4.41-4.59 (1H, m), 6.37 (1H, d, J=16.0 Hz), 6.79 (1H, d, J=2.5 Hz), 6.95 (1H, dd, J=8.8, 2.5 Hz), 7.61 (1H, d, J=8.7 Hz), 7.70 (1H, d, J=16.2 Hz), 8.01 (1H, d, J=2.3 Hz), 8.24 (1H, dd, J=2.2, 1.0 Hz).

### Reference Example 218

A mixture of ethyl (2E)-3-{2-{[3-chloro-5-(trifluoromethyl)pyridin-2-yl]oxy}-4-[2-ethoxy-1-(ethoxymethyl)ethoxy]phenyl}acrylate (1.96 g), a 1N aqueous sodium hydroxide solution (7.4 ml), tetrahydrofuran (3.5 ml) and ethanol (3.5 ml) was stirred at 50° C. for 2 hr. The reaction mixture was concentrated, 1N hydrochloric acid (7.4 ml) was added, and the mixture was extracted with ethyl acetate. The ethyl acetate layer was washed with saturated brine, dried (MgSO₄), and concentrated. The residue was recrystallized from ethyl acetate-hexane. The obtained residue was subjected to silica gel column chromatography, and eluted with ethyl acetate-hexane (1:25-1:5-1:1, v/v). The obtained crude crystals were recrystallized from ethyl acetate-hexane to give (2E)-3-{2-{[3-chloro-5-(trifluoromethyl)pyridin-2-yl]oxy}-4-[2-ethoxy-1-(ethoxymethyl)ethoxy]phenyl}acrylic acid (407 mg, yield: 23%) as colorless crystals. melting point 62-63° C.

### Reference Example 219

To a solution of ethyl (2E)-3-(2-{[3-chloro-5-(trifluoromethyl)pyridin-2-yl]oxy}-4-hydroxyphenyl)acrylate (3.00 g) in N,N-dimethylformamide (10 ml) were added potassium carbonate (2.14 g), sodium iodide (1.74 g) and 2-(2-bromoethyl)-1,3-dioxolane (1.36 ml) at room temperature, and the mixture was stirred at 80° C. for 12 hr. After cooling, water was added to the reaction mixture, and the mixture was extracted with ethyl acetate. The organic layer was washed with water and saturated brine, dried (MgSO₄), and concentrated. The obtained residue was subjected to silica gel column chromatography, and eluted with ethyl acetate-hexane (1:20-1:1, v/v). The obtained crude crystals were recrystallized from diethyl ether-hexane to give ethyl (2E)-3-{2-{[3-chloro-5-(trifluoromethyl)pyridin-2-yl]oxy}-4-[2-(1,3-dioxolan-2-yl)ethoxy]phenyl}acrylate (2.75 g, yield: 73%) as a white powder melting point 102-103° C.

### Reference Example 220

A mixture of ethyl (2E)-3-{2-{[3-chloro-5-(trifluoromethyl)pyridin-2-yl]oxy}-4-[2-(1,3-dioxolan-2-yl)ethoxy]phenyl}acrylate (2.71 g), a 1N aqueous sodium hydroxide solution (11.1 ml), tetrahydrofuran (11 ml) and ethanol (11 ml) was stirred at 50° C. for 2 hr. The reaction mixture was concentrated, 1N hydrochloric acid (11.1 ml) was added, and the mixture was extracted with ethyl acetate. The ethyl acetate layer was washed with saturated brine, dried (MgSO₄), and concentrated. The residue was recrystallized from ethyl acetate-hexane to give (2E)-3-{2-{[3-chloro-5-(trifluoromethyl)pyridin-2-yl]oxy}-4-[2-(1,3-dioxolan-2-yl)ethoxy]phenyl}acrylic acid (2.11 g, yield: 83%) as colorless crystals. The residue was subjected to silica gel column chromatography, and eluted with ethyl acetate-hexane (1:10-2:1, v/v). The obtained crude crystals were recrystallized from ethyl acetate-hexane to give colorless crystals. melting point 157-158.5° C.

### Reference Example 221

To a solution of ethyl (2E)-3-(2-{[3-chloro-5-(trifluoromethyl)pyridin-2-yl]oxy}-4-hydroxyphenyl)acrylate (4.02 g) in N,N-dimethylformamide (25 ml) were added potassium carbonate (2.87 g), sodium iodide (3.12 g) and 2-(bromomethyl)tetrahydrofuran (2.34 ml) at room temperature, and the mixture was stirred at 80° C. for 16 hr. After cooling, water was added to the reaction mixture, and extracted with ethyl acetate-hexane (1:1, v/v). The organic layer was washed with water and saturated brine, dried (MgSO₄), and concentrated. The obtained residue was subjected to silica gel column chromatography, and eluted with ethyl acetate-hexane (1:20-1:3, v/v). The obtained crude crystals were recrystallized from diisopropyl ether-hexane to give ethyl (2E)-3-[2-{[3-chloro-5-(trifluoromethyl)pyridin-2-yl]oxy}-4-(tetrahydrofuran-2-ylmethoxy)phenyl]acrylate (1.85 g, yield: 38%) as a white powder melting point 84-86° C.

### Reference Example 222

A mixture of ethyl (2E)-3-[2-{[3-chloro-5-(trifluoromethyl)pyridin-2-yl]oxy}-4-(tetrahydrofuran-2-ylmethoxy)phenyl]acrylate (1.75 g), a 1N aqueous sodium hydroxide solution (7.4 ml), tetrahydrofuran (3.5 ml) and ethanol (3.5 ml) was stirred at 50° C. for 2 hr. 1N Hydrochloric acid (7.4 ml) was added to the reaction mixture, and the mixture was extracted with ethyl acetate. The ethyl acetate layer was washed with saturated brine, dried (MgSO₄), and concentrated. The obtained residue was recrystallized from diethyl ether-hexane to give (2E)-3-[2-{[3-chloro-5-(trifluoromethyl)pyridin-2-yl]oxy}-4-(tetrahydrofuran-2-ylmethoxy)phenyl]acrylic acid (0.79 g, yield: 48%) as white crystals. melting point 106-109° C.

### Reference Example 223

To a solution of ethyl (2E)-3-(2-{[3-chloro-5-(trifluoromethyl)pyridin-2-yl]oxy}-4-hydroxyphenyl)acrylate (4.02 g) in N,N-dimethylformamide (25 ml) were added potassium carbonate (2.87 g), sodium iodide (3.12 g) and 2-chloropyrimidine (2.38 g) at room temperature, and the mixture was stirred at 80° C. for 12 hr. After cooling, water was added to the reaction mixture, and the mixture was extracted with ethyl acetate. The organic layer was washed with water and saturated brine, dried (MgSO₄), filtrated and concentrated. The obtained residue was subjected to NH-silica gel column chromatography, and eluted with ethyl acetate-hexane (1:20-1:2, v/v) to give ethyl (2E)-3-[2-{[3-chloro-5-(trifluoromethyl)pyridin-2-yl]oxy}-4-(pyrimidin-2-yloxy)phenyl]acrylate (2.50 g, yield: 54%) as a pale-yellow oil.

¹H-NMR (300 MHz, CDCl₃) δ:1.30 (3H, t, J=7.2 Hz), 4.22 (2H, q, J=7.2 Hz), 6.47 (1H, d, J=16.2 Hz), 7.04-7.13 (2H, m), 7.20 (1H, dd, J=8.7, 2.5 Hz), 7.75 (1H, d, J=3.6 Hz), 7.79 (1H, d, J=11.1 Hz), 8.01 (1H, d, J=2.1 Hz), 8.26 (1H, dd, J=2.1, 0.9 Hz), 8.59 (2H, d, J=4.9 Hz).

### Reference Example 224

A mixture of ethyl (2E)-3-[2-{[3-chloro-5-(trifluoromethyl)pyridin-2-yl]oxy}-4-(pyrimidin-2-yloxy)phenyl]acrylate (2.38 g), a 1N aqueous sodium hydroxide solution (11 ml), tetrahydrofuran (12 ml) and ethanol (12 ml) was stirred at 50° C. for 2 hr. The reaction mixture was concentrated, 1N hydrochloric acid (11 ml) was added, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:25-1:5-1:1, v/v) to give (2E)-3-[2-{[3-chloro-5-(trifluoromethyl)pyridin-2-yl]oxy}-4-(pyrimidin-2-yloxy)phenyl]acrylic acid (1.26 g, yield: 54%) as a white amorphous powder.

¹H-NMR (300 MHz, CDCl₃) δ:6.49 (1H, d, J=16.0 Hz), 7.03-7.14 (2H, m), 7.22 (1H, dd, J=8.6, 2.4 Hz), 7.78 (1H, d, J=8.7 Hz), 7.86 (1H, d, J=16.2 Hz), 8.02 (1H, d, J=2.3 Hz), 8.26 (1H, dd, J=2.1, 0.9 Hz), 8.60 (2H, d, J=4.9 Hz).

### Reference Example 225

To a mixture of methyl 3-isopropoxy-1H-pyrazole-5-carboxylate (1.55 g), potassium carbonate (1.75 g) and N,N-dimethylformamide (15 ml) was added 3-chloro-2-(chloromethyl)-5-(trifluoromethyl)pyridine (1.94 g) at 0° C., and the mixture was stirred at room temperature for 15 hr. Water was poured between water and ethyl acetate. The ethyl acetate layer was washed with water and saturated brine, dried (MgSO₄), and concentrated. The residue was subjected to silica gel column chromatography, and eluted with ethyl acetate-hexane (1:49-1:9, v/v) to give methyl 1-{[3-chloro-5-(trifluoromethyl)pyridin-2-yl]methyl}-3-isopropoxy-1H-pyrazole-5-carboxylate (1.63 g, yield: 51%) as a yellow oil.

¹H-NMR (300 MHz, CDCl₃) δ:1.32 (6H, d, J=6.3 Hz), 3.80 (3H, s), 4.63-4.76 (1H, m), 5.92 (2H, s), 6.30 (1H, s), 7.87-7.92 (1H, m), 8.59-8.63 (1H, m).

### Reference Example 226

A mixture of methyl 1-{[3-chloro-5-(trifluoromethyl)pyridin-2-yl]methyl}-3-isopropoxy-1H-pyrazole-5-carboxylate (1.92 g), a 1N aqueous sodium hydroxide solution (20 ml), tetrahydrofuran (20 ml) and methanol (10 ml) was stirred at 50° C. for 1 hr. The reaction mixture was concentrated, 1N hydrochloric acid (100 ml) was added, and the mixture was extracted with ethyl acetate. The ethyl acetate layer was washed with saturated brine, dried (MgSO₄), and concentrated. The residue was crystallized from ethyl acetate-hexane to give 1-{[3-chloro-5-(trifluoromethyl)pyridin-2-yl]methyl}-3-isopropoxy-1H-pyrazole-5-carboxylic acid (1.63 g, yield: 88%) as colorless crystals. melting point 124-125° C.

### Reference Example 227

A mixture of N,O-dimethylhydroxylamine hydrochloride (515 mg), triethylamine (534 mg) and N,N-dimethylformamide (30 ml) was stirred at room temperature for 30 min, 1-{[3-chloro-5-(trifluoromethyl)pyridin-2-yl]methyl}-3-isopropoxy-1H-pyrazole-5-carboxylic acid (1.60 g), 1-ethyl-3-(3′-dimethylaminopropyl)carbodiimide hydrochloride (1.01 g) and 1-hydroxybenzotriazole monohydrate (809 mg) were added, and the mixture was stirred at room temperature for 15 hr. The reaction mixture was poured into water, and the mixture was extracted with ethyl acetate. The extract was washed with 1N hydrochloric acid and saturated brine, dried (MgSO₄), and concentrated. The residue was subjected to silica gel column chromatography, and eluted with ethyl acetate-hexane (1:19-1:4, v/v) to give 1-{[3-chloro-5-(trifluoromethyl)pyridin-2-yl]methyl}-3-isopropoxy-N-methoxy-N-methyl-1H-pyrazole-5-carboxamide (1.37 g, yield: 77%) as a colorless oil.

¹H-NMR (300 MHz, CDCl₃) δ:1.33 (6H, d, J=6.0 Hz), 3.28 (3H, s), 3.72 (3H, s), 4.67-4.80 (1H, m), 5.92 (2H, s), 6.30 (1H, s), 7.86-7.90 (1H, m), 8.53-8.57 (1H, m).

### Reference Example 228

To a solution of 1-{[3-chloro-5-(trifluoromethyl)pyridin-2-yl]methyl}-3-isopropoxy-N-methoxy-N-methyl-1H-pyrazole-5-carboxamide (1.36 g) in tetrahydrofuran (25 ml) was added a 1.5 M diisobutylaluminum hydride solution in toluene (3.4 ml) at 0° C., and the mixture was stirred for 2 hr. Furthermore, to the reaction mixture was added a 1.5 M diisobutylaluminum hydride solution in toluene (1.5 ml) at 0° C., and the mixture was stirred for 30 min. Methanol was added to quench the reaction. The reaction mixture was poured into a 10% aqueous Rochelle salt solution, and the mixture was stirred and extracted with ethyl acetate. The extract was washed with 1N hydrochloric acid and saturated brine, dried (MgSO₄), and concentrated. The residue was subjected to silica gel column chromatography, and eluted with ethyl acetate-hexane (1:24-1:4, v/v) to give 1-{[3-chloro-5-(trifluoromethyl)pyridin-2-yl]methyl}-3-isopropoxy-1H-pyrazole-5-carbaldehyde (640 mg, yield: 55%) as a brown oil.

¹H-NMR (300 MHz, CDCl₃): 1.34 (6H, d, J=6.0 Hz), 4.68-4.81 (1H, m), 5.87 (2H, s), 6.35 (1H, s), 7.88-7.92 (1H, m), 8.56-8.61 (1H, m), 9.74 (1H, s).

### Reference Example 229

Under ice-cooling, to a solution of ethyl diethylphosphonoacetate (619 mg) in N,N-dimethylformamide (6.0 ml) was added sodium hydride (60% in oil, 96 mg), and the mixture was stirred at room temperature for 30 min. The reaction mixture was ice-cooled again, a solution of 1-{[3-chloro-5-(trifluoromethyl)pyridin-2-yl]methyl}-3-isopropoxy-1H-pyrazole-5-carbaldehyde (640 mg) in tetrahydrofuran (6.0 ml) was added, and the mixture was stirred at DOC for 2 hr. A saturated aqueous ammonium chloride solution was added to the reaction mixture, and the mixture was extracted with ethyl acetate. The ethyl acetate layer was washed with water and saturated brine, dried (MgSO₄), and concentrated. The residue was crystallized from ethyl acetate-hexane to give ethyl (2E)-3-(1-{[3-chloro-5-(trifluoromethyl)pyridin-2-yl]methyl}-3-isopropoxy-1H-pyrazol-5-yl)acrylate (640 mg, yield: 83%) as a pale-yellow oil.

¹H-NMR (300 MHz, CDCl₃) δ:1.31 (3H, t, J=7.2 Hz), 1.31 (6H, d, J=6.0 Hz); 4.23 (2H, d, J=7.2 Hz), 4.61-4.74 (1H, m), 5.56 (2H, s), 6.02 (1H, s), 6.32 (1H, d, J=15.6 Hz), 7.48 (1H, d, J=15.6 Hz), 7.91-7.95 (1H, m), 8.65-8.70 (1H, m).

### Reference Example 230

A mixture of methyl (2E)-3-(1-{[3-chloro-5-(trifluoromethyl)pyridin-2-yl]methyl}-3-isopropoxy-1H-pyrazol-5-yl)acrylate (640 mg), a 1N aqueous sodium hydroxide solution (3.0 ml), tetrahydrofuran (6.0 ml) and ethanol (6.0 ml) was stirred at 50° C. for 1 hr, 1N hydrochloric acid (20 ml) was added, and the mixture was extracted with ethyl acetate. The ethyl acetate layer was washed with saturated brine, dried (MgSO₄), and concentrated. The obtained crude crystals were recrystallized from hexane-ethyl acetate to give (2E)-3-(1-{[3-chloro-5-(trifluoromethyl)pyridin-2-yl]methyl}-3-isopropoxy-1H-pyrazol-5-yl)acrylic acid (460 mg, yield: 77%) as colorless crystals. melting point 130-132° C.

### Reference Example 231

To a solution of benzyl alcohol (3.06 g) in dichloromethane (150 ml) was added chlorosulfonylisocyanate (2.55 ml) under ice-cooling, and the mixture was stirred for 30 min. Pyridine (8.0 ml) was added to the reaction mixture, and the mixture was stirred for 1 hr. 4-Methylcyclohexylamine (18.0 ml) was added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated to give a pale-yellow solid. Recrystallization from ethyl acetate-hexane gave benzyl {[(4-methylcyclohexyl)amino]sulfonyl}carbamate (6.64 g, yield: 72%) as white crystals. Recrystallization from ethyl acetate-diisopropyl ether gave white feather crystals. melting point 153-155° C.

### Reference Example 232

To a solution of benzyl {[(4-methylcyclohexyl)amino]sulfonyl}carbamate (6.34 g) in tetrahydrofuran (40 ml) and ethanol (40 ml) was added 10% palladium-carbon (3.41 g), and the mixture was stirred under a hydrogen atmosphere at room temperature for 3 hr. The reaction mixture was filtrated, and the filtrate was concentrated. Recrystallization of the obtained residue from ethyl acetate-hexane gave N-(4-methylcyclohexyl)sulfamide (3.19 g, yield: 86%) as white mica crystals. melting point 109-110° C.

### Reference Example 233

To a solution of benzyl alcohol (3.02 g) in dichloromethane (150 ml) was added chlorosulfonylisocyanate (2.55 ml) under ice-cooling, and the mixture was stirred for 30 min. Pyridine (8.0 ml) was added to the reaction mixture, and the mixture was stirred for 1 hr. 2-(2-Thienyl)ethylamine (5.0 g) was added, and the mixture was stirred overnight at room temperature. 1N Hydrochloric acid was added to the reaction mixture, and the mixture was extracted with ethyl acetate. The organic layer was washed with 1N hydrochloric acid, a saturated aqueous sodium hydrogencarbonate solution and saturated brine, dried (MgSO₄), filtrated and concentrated. The obtained residue was washed with diisopropyl ether. Recrystallization from ethyl acetate-hexane gave benzyl ({[2-(2-thienyl)ethyl]amino}sulfonyl)carbamate (8.29 g, yield: 87%) as white crystals. Recrystallization from ethyl acetate-diisopropyl ether gave white crystals. melting point 129-131° C.

### Reference Example 234

A solution of benzyl ({[2-(2-thienyl)ethyl]amino}sulfonyl)carbamate (6.34 g) in 25% w/w hydrobromic acid-acetic acid (25 ml) and acetic acid (10 ml) was stirred at room temperature for 1 hr, and then at 45° C. for 30 min. The reaction mixture was poured into an ice-cooled saturated aqueous sodium hydrogencarbonate solution, and diluted with ethyl acetate. The organic layer was washed with water and saturated brine, filtrated, dried (MgSO₄), filtrated and concentrated, and the filtrate was concentrated. The obtained residue was subjected to basic silica gel column chromatography, and eluted with ethyl acetate-hexane (15:85-1:1, v/v) to give a pale brown solid. Recrystallization from ethyl acetate-hexane gave N-[2-(2-thienyl)ethyl]sulfamide (3.21 g, yield: 66%) as a white solid. Recrystallization from ethyl acetate-diisopropyl ether gave white crystals.

¹H-NMR (300 MHz, CDCl₃) δ:3.13 (2H, t, J=6.6 Hz), 3.43 (2H, q, J=6.5 Hz), 4.36-4.60 (3H, m), 6.89 (1H, dd, J=3.5, 1.0 Hz), 6.96 (1H, dd, J=5.2, 3.5 Hz), 7.19 (1H, dd, J=5.2, 1.2 Hz)

### Reference Example 235

To a solution of ethyl (2E)-3-[2-{[3-chloro-5-(trifluoromethyl)pyridin-2-yl]oxy}-4-(2-methoxyethoxy)phenyl]-2-methylacrylate (3.00 g) in tetrahydrofuran (30 ml) was added 5% palladium-carbon (1.51 g), and the mixture was stirred under a hydrogen atmosphere at room temperature for 3 hr. The reaction mixture was filtrated, and the filtrate was concentrated. The obtained residue was subjected to basic silica gel column chromatography, and eluted with ethyl acetate-hexane (hexane alone to 2:3, v/v) to give a colorless oil.

To a solution of the obtained oil in tetrahydrofuran (5 ml) and ethanol (5 ml) was added a 1N aqueous sodium hydroxide solution (15 ml), and the mixture was stirred at 50° C. for 30 min. After allowing to cool to room temperature, 1N hydrochloric acid (15 ml) and a small amount of toluene were added to the reaction mixture; and the mixture was concentrated. The obtained residue was dissolved in ethyl acetate, and the solution was washed with saturated brine, dried (MgSO₄), filtrated and concentrated. The obtained residue was recrystallized from ethyl acetate-hexane to give 3-[2-{[3-chloro-5-(trifluoromethyl)pyridin-2-yl]oxy}-4-(2-methoxyethoxy)phenyl]-2-methylpropanoic acid (1.25 g, yield: 52%) as white crystals. melting point 129.5-131.0° C.

### Reference Example 236

To a solution of 2-(benzyloxy)-4-(2-methoxyethoxy)benzaldehyde (11.92 g) in tetrahydrofuran (80 ml) were added methyl methoxyacetate (6.16 g) and potassium t-butoxide (6.81 g), and the mixture was stirred at room temperature for 5 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (hexane alone to 3:2, v/v) to give a yellow oil.

To a solution of the obtained oil in methanol (100 ml) was added 10% palladium-carbon (5.01 g), and the mixture was stirred under a hydrogen atmosphere at room temperature for 5 hr. The reaction mixture was filtrated, and the filtrate was concentrated. The obtained residue was subjected to silica gel column chromatography, and eluted with ethyl acetate-hexane (1:19-45:55, v/v) to give methyl 3-[2-hydroxy-4-(2-methoxyethoxy)phenyl]-2-methoxypropanoate (3.45 g, yield: 29%) as a yellow oil.

¹H-NMR (300 MHz, CDCl₃) δ:2.91-3.11 (2H, m)), 3.44 (3H, s), 3.49 (3H, s), 3.70-3.77 (5H, m), 4.01-4.12 (3H, m), 6.43 (1H, dd, J=8.3, 2.6 Hz), 6.51 (1H, d, J=2.6 Hz), 6.91 (1H, d, J=8.3 Hz), 7.69 (1H, s).

### Reference Example 237

To a solution of methyl 3-[2-hydroxy-4-(2-methoxyethoxy)phenyl]-2-methoxypropanoate (1.84 g) in N,N-dimethylformamide (30 ml) was added under ice-cooling sodium hydride (60% in oil, 311 mg), and the mixture was subsequently stirred for 30 min. Then, 2,3-dichloro-5-(trifluoromethyl)pyridine (1.0 ml) was added, and the mixture was stirred at room temperature for 30 min. A saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (hexane alone to 2:3, v/v) to give methyl 3-[2-{[3-chloro-5-(trifluoromethyl)pyridin-2-yl]oxy}-4-(2-methoxyethoxy)phenyl]-2-methoxypropanoate (2.26 g, yield: 75%) as a yellow oil.

¹H-NMR (300 MHz, CDCl₃) δ:2.76-2.94 (2H, m), 3.28 (3H, s), 3.44 (3H, s), 3.67 (3H, s), 3.71-3.76 (2H, m), 3.95 (1H, dd, J=7.9, 5.5 Hz), 4.07-4.12 (2H, m), 6.69-6.72 (1H, m), 6.82 (1H, dd, J=8.6, 2.5 Hz), 7.22-7.30 (1H, m), 7.99 (1H, d, J=1.9 Hz), 8.26 (1H, dd, J=2.3, 0.9 Hz).

### Reference Example 238

To a solution of methyl 3-[2-{[3-chloro-5-(trifluoromethyl)pyridin-2-yl]oxy}-4-(2-methoxyethoxy)phenyl]-2-methoxypropanoate (2.26 g) in tetrahydrofuran (5 ml) and methanol (5 ml) was added a 1N aqueous sodium hydroxide solution (15 ml), and the mixture was stirred at 50° C. for 30 min. After allowing to cool to room temperature, 1N hydrochloric acid (15 ml) and a small amount of toluene were added to the reaction mixture, and the mixture was concentrated. The obtained residue was dissolved in ethyl acetate, and the mixture was washed with saturated brine, dried (MgSO₄), filtrated and concentrated. The obtained residue was subjected to silica gel column chromatography, and eluted with ethyl acetate-hexane (1:4 alone to 65:35, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]-2-methoxypropanoic acid (1.19 g, yield: 54%) as white crystals. melting point 93-96° C.

### Reference Example 239

To a solution of ethyl (2E)-3-[2-{[3-chloro-5-(trifluoromethyl)pyridin-2-yl]oxy}-4-(2-methoxyethoxy)phenyl]-2-methylacrylate (2.37 g) in tetrahydrofuran (10 ml) and ethanol (10 ml) was added a 1N aqueous sodium hydroxide solution (15 ml), and the mixture was stirred at 50° C. for 4 hr. A 1N aqueous sodium hydroxide solution (15 ml) was added to the reaction mixture, and the mixture was further stirred for 3 hr. After allowing to cool to room temperature, 1N hydrochloric acid (30 ml) was added to the reaction mixture, and the mixture was diluted with toluene and concentrated. The residue was dissolved in ethyl acetate, and the organic layer was washed with saturated brine, dried (MgSO₄), filtrated and concentrated. The obtained residue was recrystallized from ethyl acetate-hexane to give a white solid. The obtained solid was subjected to silica gel column chromatography, and eluted with ethyl acetate-hexane (35:65-7:3, v/v) to give a white solid. Recrystallization from ethyl acetate-hexane gave (2E)-3-[2-{[3-chloro-5-(trifluoromethyl)pyridin-2-yl]oxy}-4-(2-methoxyethoxy)phenyl]-2-methylacrylic acid (741 mg, yield: 33%) as white needles melting point 122.0-122.5° C.

### Reference Example 240

To a solution of [2-{[3-chloro-5-(trifluoromethyl)pyridin-2-yl]oxy}-4-(2-methoxyethoxy)phenyl]methanol (9.92 g) in tetrahydrofuran (60 ml) and diethyl ether (240 ml) were added pyridine (0.50 ml) and thionyl chloride (4.5 ml) under ice-cooling, and the mixture was stirred at room temperature for 3 hr. Water was added to the reaction mixture and the mixture was extracted with ethyl acetate. The organic layer was washed with 1N hydrochloric acid, a saturated aqueous sodium hydrogencarbonate solution, and saturated brine, dried (MgSO₄), filtrated and concentrated. The obtained residue was subjected to silica gel column chromatography, and eluted with ethyl acetate-hexane (hexane alone to 1:9, v/v) to give 3-chloro-2-[2-(chloromethyl)-5-(2-methoxyethoxy)phenoxy]-5-(trifluoromethyl)pyridine (8.06 g, yield: 77%) as a white solid. Recrystallization from ethyl acetate-hexane gave white crystals. melting point 108-111° C.

### Reference Example 241

To a solution of benzyl alcohol (3.06 g) in dichloromethane (150 ml) was added chlorosulfonyl isocyanate (2.55 ml) under ice-cooling, and the mixture was stirred for 30 min. Pyridine (8.0 ml) was added to the reaction mixture, and the mixture was stirred for 1 hr. 1-Pentylamine (16.0 ml) was added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated to give a pale-yellow solid. Recrystallization from ethyl acetate-hexane gave benzyl [(pentylamino)sulfonyl]carbamate (8.18 g, yield: 96%) as white crystals. melting point 142.5-143.0° C.

### Reference Example 242

To a solution of benzyl [(pentylamino)sulfonyl]carbamate (5.83 g) in tetrahydrofuran (50 ml) and ethanol (50 ml) was added 10% palladium-carbon (3.11 g), and the mixture was stirred under a hydrogen atmosphere at room temperature for 4 hr. The reaction mixture was filtrated, and the filtrate was concentrated. The obtained residue was recrystallized from ethyl acetate-diisopropyl ether to give N-pentylsulfamide (3.15 g, yield: 98%) as white mica crystals. melting point 60-63° C.

### Reference Example 243

To a solution of ethyl diphenylphosphonoacetate (1.08 g) in tetrahydrofuran (50 ml) was added a Triton B (trade name) 40%-methanol solution (1.60 ml) at −78° C., 15 min later, a solution of 2-{[3-chloro-5-(trifluoromethyl)pyridin-2-yl]oxy}-4-(2-methoxyethoxy)benzaldehyde (1.15 g) fin tetrahydrofuran (15 ml) was added dropwise, and the mixture was stirred for 30 min. A saturated aqueous ammonium chloride solution was added to the reaction mixture, and the mixture was diluted with ethyl acetate. The organic layer was washed with water and saturated brine, dried (MgSO₄), filtrated and concentrated. The obtained residue was subjected to silica gel column chromatography, and eluted with ethyl acetate-hexane (hexane alone to 35:65, v/v) to give ethyl (2Z)-3-[2-{[3-chloro-5-(trifluoromethyl)pyridin-2-yl]oxy}-4-(2-methoxyethoxy)phenyl]acrylate (1.12 g, yield: 82%) as a pale-yellow oil.

¹H-NMR (300 MHz, CDCl₃) δ:1.23 (3H, t, J=7.1 Hz), 3.43 (3H, s), 3.72-3.77 (2H, m), 4.08-4.19 (4H, m), 5.83 (1H, d, J=12.4 Hz), 6.70 (1H, d, J=2.2 Hz), 6.83 (1H, d, J=12.4 Hz), 6.83°-6.89 (1H, m), 7.78 (1H, d, J=8.8 Hz), 7.96 (1H, d, J=1.9 Hz), 8.24 (1H, dd, J=2.1, 1.0 Hz).

### Reference Example 244

To a solution of ethyl (2Z)-3-[2-{[3-chloro-5-(trifluoromethyl)pyridin-2-yl]oxy}-4-(2-methoxyethoxy)phenyl]acrylate (1.12 g) in tetrahydrofuran (4 ml), ethanol (4 ml) and water (4 ml) was added lithium hydroxide monohydrate (256 mg) under ice-cooling, and the mixture was stirred for 1 hr. Under ice-cooling, lithium hydroxide monohydrate (412 mg) was further added, and the mixture was stirred for 30 min. 1N Hydrochloric acid (16 ml) was added to the reaction mixture, and the mixture was diluted with ethyl acetate. The organic layer was washed with saturated brine, dried (MgSO₄), filtrated and concentrated. The obtained solid was recrystallized from ethyl acetate-hexane to give (2Z)-3-[2-{[3-chloro-5-(trifluoromethyl)pyridin-2-yl]oxy}-4-(2-methoxyethoxy)phenyl]acrylic acid (780 g, yield: 74%) as white crystals. melting point 131.8-132.0° C.

### Reference Example 245

To a solution of ethyl (2E)-3-(2-{[3-chloro-5-(trifluoromethyl)pyridin-2-yl]oxy}-4-hydroxyphenyl)acrylate (5.00 g) in tetrahydrofuran (100 ml) were added tributylphosphine (5.40 ml), 2-{[tert-butyl(diphenyl)silyl]oxy}ethanol (5.82 g) and 1,1′-(azodicarbonyl)dipiperidine (4.97 g), and the mixture was stirred overnight at 50° C. The reaction mixture was concentrated, the obtained solid was washed with diisopropyl ether, and the filtrate was concentrated. The obtained residue was subjected to silica gel column chromatography, and eluted with ethyl acetate-hexane (hexane alone to 3:7, v/v) to give ethyl (2E)-3-(4-(2-{[tert-butyl(diphenyl)silyl]oxy}ethoxy)-2-{[3-chloro-5-(trifluoromethyl)pyridin-2-yl]oxy}phenyl)acrylate (7.51 g, yield: 87%) as a white oil.

¹H-NMR (300 MHz, CDCl₃) δ:1.04 (9H, s), 1.22-1.29 (3H, m), 3.98 (2H, t, J=4.9 Hz), 4.06-4.15 (2H, m), 4.15-4.27 (2H, m), 6.36 (1H, d, J=16.0 Hz), 6.65 (1H, d, J=2.4 Hz), 6.83 (1H, dd, J=8.7, 2.4 Hz), 7.32-7.49 (6H, m), 7.61 (1H, d, J=8.9 Hz), 7.65-7.78 (5H, m), 8.01 (1H, d, J=2.3 Hz), 8.23 (1H, dd, J=2.1, 0.9 Hz).

### Reference Examples 246, 247

To a solution of ethyl (2E)-3-(4-(2-{[tert-butyl (diphenyl)silyl]oxy}ethoxy)-2-{[3-chloro-5-(trifluoromethyl)pyridin-2-yl]oxy}phenyl)acrylate (7.02 g) in tetrahydrofuran (10 ml) and ethanol (10 ml) was added a 1N aqueous sodium hydroxide solution (25 ml) at room temperature, and the mixture was stirred for 4 hr. 1N Hydrochloric acid was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (hexane alone to 3:7, v/v) to give (2E)-3-(4-(2-{[tert-butyl(diphenyl)silyl]oxy}ethoxy)-2-{[3-chloro-5-(trifluoromethyl)pyridin-2-yl]oxy}phenyl)acrylic acid (789 mg yield: 12%) (Reference Example 246) as a colorless oil.

¹H-NMR (300 MHz, CDCl₃) δ:1.04 (9H, s), 3.98 (2H, t, J=5.2 Hz), 4.10 (2H, t, J=4.7 Hz), 6.37 (1H, d, J=16.0 Hz), 6.64 (1H, d, J=2.4 Hz), 6.84 (1H, dd, J=8.7, 1.7 Hz), 7.30-7.47 (6H, m), 7.62 (1H, d, J=8.7 Hz), 7.68 (4H, dd, J=7.8, 1.6 Hz), 7.75 (1H, d, J=16.0 Hz), 8.01 (1H, d, J=2.1 Hz), 8.06-8.30 (1H, m).

Then, a white solid was obtained. Recrystallization from ethyl acetate-hexane gave (2E)-3-[2-{[3-chloro-5-(trifluoromethyl)pyridin-2-yl]oxy}-4-(2-hydroxyethoxy)phenyl]acrylic acid monohydrate (132 mg, yield: 3%) (Reference Example 247) as white crystals. melting point 153.0-153.2° C.

### Reference Example 248

To a solution of ethyl (2E)-3-[2-hydroxy-4-(2-methoxyethoxy)phenyl]acrylate (2.60 g) in N,N-dimethylformamide (20 ml) were added potassium carbonate (2.20 g) and 4-fluoronitrobenzene (1.50 ml), and the mixture was stirred overnight at room temperature, and then at 50° C. for 30 min. After allowing to cool to room temperature, 1N hydrochloric acid was added to the reaction mixture, and the mixture was extracted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give ethyl (2E)-3-[4-(2-methoxyethoxy)-2-(4-nitrophenoxy)phenyl]acrylate (3.97 g, quant.) as a yellow solid. Recrystallization from ethyl acetate-hexane gave pale-yellow crystals as 0.3 hydrate melting point 61-64° C.

### Reference Example 249

To a solution of ethyl (2E)-3-[4-(2-methoxyethoxy)-2-(4-nitrophenoxy)phenyl]acrylate (3.89 g) in ethanol (50 ml) and tetrahydrofuran (10 ml) was added 10% palladium-carbon (0.91 g), and the mixture was stirred under a hydrogen atmosphere at room temperature for 1 hr. The reaction mixture was filtrated and the filtrate was concentrated to give ethyl (2E)-3-[2-(4-aminophenoxy)-4-(2-methoxyethoxy)phenyl]acrylate (2.97 g, yield: 84%) as a pale-brown oil.

¹H-NMR (300 MHz, CDCl₃) δ:1.22 (3H, t, J=7.1 Hz), 2.56-2.72 (2H, m), 2.94 (2H, t, J=7.7 Hz), 3.40 (3H, s), 3.57 (2H, s), 3.63-3.71 (2H, m), 3.93-3.99 (2H, m), 4.10 (2H, q, J=7.1 Hz), 6.31 (1H, d, J=2.5 Hz), 6.51 (1H, dd, J=8.5, 2.5 Hz), 6.61-6.71 (2H, m), 6.74-6.88 (2H, m), 7.09 (1H, d, J=8.2 Hz).

### Reference Example 250

To a solution of ethyl (2E)-3-(2-{[3-chloro-5-(trifluoromethyl)pyridin-2-yl]oxy}-4-hydroxyphenyl)-2-methylacrylate (4.61 g) in N,N-dimethylformamide (20 ml) were added potassium carbonate (3.12 g) and isopropyl iodide (2.0 ml), and the mixture was stirred at 50° C. for 1 hr. After allowing to, cool to room temperature, water was added to the reaction mixture and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (hexane alone to 2:3, v/v) to give ethyl (2E)-3-(2-{[3-chloro-5-(trifluoromethyl)pyridin-2-yl]oxy}-4-isopropoxyphenyl)-2-methylacrylate (3.46 g, yield: 68%) as white crystals. Recrystallization from ethyl acetate-hexane gave white crystals. melting point 61.0-62.0° C.

### Reference Example 251

To a solution of ethyl (2E)-3-(2-{[3-chloro-5-(trifluoromethyl)pyridin-2-yl]oxy}-4-isopropoxyphenyl)-2-methylacrylate (3.46 g) in tetrahydrofuran (10 ml) and ethanol (10 ml) was added a 1N aqueous sodium hydroxide solution (20 ml), and the mixture was stirred at 50° C. for 2 hr. After allowing to cool to room temperature, 1N hydrochloric acid (20 ml) was added to the reaction mixture, and the mixture was concentrated and diluted with ethyl acetate. The organic layer was washed with water and saturated brine, dried (MgSO₄), filtrated and concentrated. The obtained residue was subjected to silica gel column chromatography, and eluted with ethyl acetate-hexane (3:7-7:3, v/v) to give a white solid. Recrystallization from ethyl acetate-hexane gave (2E)-3-(2-{[3-chloro-5-(trifluoromethyl)pyridin-2-yl]oxy}-4-isopropoxyphenyl)-2-methylacrylic acid (2.02 g, yield: 62%) as white crystals. melting point 114.4-114.5° C.

### Reference Example 252

To a solution of benzyl alcohol (3.05 g) in dichloromethane (60 ml) was added chlorosulfonyl isocyanate (2.50 ml) under ice-cooling, and the mixture was stirred for 30 min. Pyridine (8.0 ml) was added to the reaction mixture, and the mixture was stirred for 1 hr. 2-Phenylethylamine (8.0 ml) was added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated to give a pale-yellow solid. Recrystallization from ethyl acetate-hexane gave benzyl {[(2-phenylethyl)amino]sulfonyl}carbamate (8.27 g, yield: 88%) as white crystals. Recrystallization from ethyl acetate-hexane gave white crystals. melting point 120.0-121.0° C.

### Reference Example 253

To a solution of benzyl {[(2-phenylethyl)amino]sulfonyl}carbamate (7.97 g) in ethanol (100 ml) was added 10% palladium-carbon (7.51 g), and the mixture was stirred overnight under a hydrogen atmosphere at room temperature. The reaction mixture was filtrated, and the filtrate was concentrated. Recrystallization of the obtained residue from ethyl acetate-hexane gave N-(2-phenylethyl)sulfamide (4.45 g, yield: 93%) as white crystals melting point 61.8-62.0° C.

### Reference Example 254

To a solution of benzyl alcohol (3.05 g) in dichloromethane (60 ml) was added chlorosulfonyl isocyanate (2.50 ml) under ice-cooling, and the mixture was stirred for 30 min. Pyridine (8.0 ml) was added to the reaction mixture, and the mixture was stirred for 1 hr. 3-Methoxypropylamine (8.0 ml) was added, and the mixture was stirred overnight at room temperature. 1N Hydrochloric acid was added to the reaction mixture, and, the mixture was diluted with ethyl acetate. The organic layer was washed with 1N hydrochloric acid, a saturated aqueous sodium hydrogencarbonate solution and saturated brine, dried (MgSO₄), filtrated and concentrated to give a white solid. Recrystallization from ethyl acetate-hexane gave benzyl {[(3-methoxypropyl)amino]sulfonyl}carbamate (1.14 g, yield: 13%) as white crystals. melting point 91-93° C.

### Reference Example 255

To a solution of benzyl {[(3-methoxypropyl)amino]sulfonyl}carbamate (1.11 g) in ethanol (5 ml) was added 10% palladium-carbon (1.22 g), and the mixture was stirred overnight under a hydrogen atmosphere at room temperature. The reaction mixture was filtrated and the filtrate was concentrated to give N-(3-methoxypropyl)sulfamide (631 mg, quant.) as a pale-yellow oil.

¹H-NMR (300 MHz, CDCl₃) δ:1.86 (2H, tt, J=5.9, 5.9 Hz), 3.26 (2H, t, J=6.2 Hz), 3.34 (3H, s), 3.52 (2H, t, J=5.7 Hz), 4.69 (2H, br. s.).

### Reference Example 256

To a solution of benzyl alcohol (3.05 g) in dichloromethane (60 ml) was added chlorosulfonyl isocyanate (2.50 ml) under ice-cooling, and the mixture was stirred for 30 min. Pyridine (8.0 ml) was added to the reaction mixture, and the mixture was stirred for 1 hr. 2-Isopropoxyethylamine (8.0 ml) was added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated to give a white solid. Recrystallization from ethyl acetate-hexane gave benzyl {[(2-isopropoxyethyl)amino]sulfonyl}carbamate (7.91 g, yield: 89%) as white crystals. melting point 88-89° C.

### Reference Example 257

To a solution of benzyl {[(2-isopropoxyethyl)amino]sulfonyl}carbamate (7.76 g) in ethanol (50 ml) was added 10% palladium-carbon (7.01 g), and the mixture was stirred overnight under a hydrogen atmosphere at room temperature. The reaction mixture was filtrated and the filtrate was concentrated to give N-(2-isopropoxyethyl)sulfamide (4.44 g, yield: 99%) as a colorless oil.

¹H-NMR (300 MHz, CDCl₃) δ:1.17 (6H, d, J=6.0 Hz), 3.24-3.37 (2H, m), 3.49-3.72 (3H, m), 4.30-5.00 (3H, m).

### Reference Example 258

To a solution of benzyl alcohol (5.41 g) in acetonitrile (500 ml) was added chlorosulfonyl isocyanate (4.40 ml) under ice-cooling, and the mixture was stirred for 30 min. Pyridine (12.0 ml) was added to the reaction mixture, and the mixture was stirred for 1 hr. 1-(Tetrahydrofuran-2-yl)methanamine (10.5 ml) was added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated to give a white solid. Recrystallization from ethyl acetate-hexane gave benzyl {[(tetrahydrofuran-2-ylmethyl)amino]sulfonyl}carbamate (14.7 g, yield: 94%) as white crystals. melting point 96-97° C.

### Reference Example 259

To a solution of benzyl {[(tetrahydrofuran-2-ylmethyl)amino]sulfonyl}carbamate (14.2 g) in ethanol (200 ml) was added 10% palladium-carbon (5.21 g), and the mixture was stirred under a hydrogen atmosphere at room temperature for 5 hr. The reaction mixture was filtrated and the filtrate was concentrated to give N-(tetrahydrofuran-2-ylmethyl)sulfamide (8.35 g, quant.) as a colorless oil.

¹H-NMR (300 MHz, CDCl₃) δ:1.52-1.70 (1H, m), 1.85-2.08 (3H, m), 3.07-3.20 (1H, m), 3.24-3.34 (1H, m), 3.72-3.83 (1H, m), 3.88 (1H, ddd, J 8.3, 6.7 Hz), 4.05-4.13 (1H, m), 4.77 (3H, br. s.).

### Reference Example 260

A solution of 3-(methylthio)propan-1-ol (5.30 g), triethylamine (10.5 ml) and N,N,N′,N′-tetramethyl-1,6-hexanediamine (0.86 g) in toluene (50 ml) was ice-cooled, and p-toluenesulfonyl chloride (14.3 g) in toluene (50 ml) was added dropwise to the solution under a nitrogen atmosphere. After completion of the dropwise addition, and the mixture was allowed to warm to room temperature and stirred for 3 hr. Water was added to the reaction mixture, and the mixture was extracted with ethyl acetate. The extract was washed with saturated brine, dried (Na₂SO₄), and concentrated under reduced pressure. The obtained residue was subjected to silica gel column chromatography, and eluted with ethyl acetate-hexane (1:9-2:3, v/v) to give 3-(methylthio)propyl 4-methylbenzenesulfonate (12.2 g, yield: 94%) as a colorless oil. MS m/z 261 (MH⁺)

### Reference Example 261

To a solution of 3-(methylthio)propyl 4-methylbenzenesulfonate (12.2 g) in methanol (250 ml) was added dropwise a solution of Oxone (trade name) (57.7 g) in water (250 ml) under ice-cooling. After completion of the dropwise addition, and the mixture was stirred for 20 hr while allowing to warm to room temperature. Methanol was evaporated under reduced pressure, and the mixture was diluted with water. The organic product was extracted with ethyl acetate. The extract was washed with saturated brine, dried (Na₂SO₄), and concentrated under reduced pressure. The precipitated crystals were washed with ethyl acetate-heptane to give 3-(methylsulfonyl)propyl 4-methylbenzenesulfonate (13.1 g, yield: 96%) as colorless crystals. MS m/z 293 (MH⁺).

### Reference Example 262

To a solution of ethyl (2E)-3-(2-{[3-chloro-5-(trifluoromethyl)pyridin-2-yl]oxy}-4-hydroxyphenyl)acrylate (5.00 g) in N,N-dimethylformamide (50 ml) were added potassium carbonate (2.67 g) and 3-(methylsulfonyl)propyl 4-methylbenzenesulfonate (4.46 g), and the mixture was stirred at 50° C. for 3 hr. After allowing to cool to room temperature, 1N hydrochloric acid was added to the reaction mixture, the resulting solid was collected by filtration and washed with water to give ethyl (2E)-3-{2-{[3-chloro-5-(trifluoromethyl)pyridin-2-yl]oxy}-4-[3-(methylsulfonyl)propoxy]phenyl}acrylate (6.34 g, yield: 97%) as a white solid. Recrystallization from ethyl acetate-hexane gave white crystals. melting point 190.6-191.0° C.

### Reference Example 263

To a solution of ethyl (2E)-3-{2-{[3-chloro-5-(trifluoromethyl)pyridin-2-yl]oxy}-4-[3-(methylsulfonyl)propoxy]phenyl}acrylate (6.14 g) in tetrahydrofuran (30 ml) and ethanol (30 ml) was added a 1N aqueous sodium hydroxide solution (30 ml), and the mixture was stirred at 50° C. for 3 hr. After allowing to cool to room temperature, 1N hydrochloric acid (30 ml) was added to the reaction mixture, and the resulting solid was collected by filtration and washed with water to give a white solid. The solid was recrystallized from aqueous ethanol to give (2E)-3-{2-{[3-chloro-5-(trifluoromethyl)pyridin-2-yl]oxy}-4-[3-(methylsulfonyl)propoxy]phenyl}acrylic acid (4.83 g, yield: 83%) as white crystals. melting point 202-203° C.

### Reference Examples 264, 265

To a solution of ethyl (2E)-3-(2-{[3-chloro-5-(trifluoromethyl)pyridin-2-yl]oxy}-4-hydroxyphenyl)acrylate (10.0 g) in tetrahydrofuran (300 ml) were added tributylphosphine (10.5 ml), 3-methylbutane-1,3-diol (4.0 ml) and 1,1′-(azodicarbonyl)dipiperidine (9.76 g), and the mixture was stirred overnight at 50° C. Then, tributylphosphine (10.5 ml), 3-methylbutane-1,3-diol (4.0 ml) and 1,1′-(azodicarbonyl)dipiperidine (9.76 g) were added, and the mixture was stirred at 50° C. for 1 hr. The reaction mixture was concentrated, the obtained solid was washed with diisopropyl ether, and the filtrate was concentrated. The obtained residue was subjected to basic silica gel column chromatography, and eluted with ethyl acetate-hexane (hexane alone to 45:55, v/v). The obtained residue was subjected to silica gel column chromatography, and eluted with ethyl acetate-hexane (hexane alone to 3:7, v/v) to give ethyl (2E)-3-[2-{[3-chloro-5-(trifluoromethyl)pyridin-2-yl]oxy}-4-(3-hydroxy-3-methylbutoxy)phenyl]acrylate (Reference Example 264) (1.46 g, yield: 12%) as a pale-yellow solid. Recrystallization from ethyl acetate-hexane gave white crystals as 0.5 hydrate melting point 63.5-66.0° C.

Then, ethyl (2E)-3-[2-{[3-chloro-5-(trifluoromethyl)pyridin-2-yl]oxy}-4-(3-hydroxy-1,1-dimethylpropoxy)phenyl]acrylate (Reference Example 265) (0.71 g, yield: 6%) was obtained as an orange oil.

¹H-NMR (300 MHz, DMSO-d₆) δ:1.20 (3H, t, J=7.2 Hz), 1.32 (6H, s), 1.87 (2H, t, J=7.5 Hz), 3.55-3.62 (2H, m), 4.12 (2H, q, J=7.2 Hz), 4.40 (1H, t, J=5.4 Hz), 6.57 (1H, d, J=16.2 Hz), 6.96 (1H, s), 6.94-6.99 (1H, m), 7.55 (1H, d, J=16.2 Hz), 8.50-8.50 (1H, m), 8.63 (1H, d, J=2.0 Hz).

### Reference Example 266

To a solution of ethyl (2E)-3-[2-{[3-chloro-5-(trifluoromethyl)pyridin-2-yl]oxy}-4-(3-hydroxy-3-methylbutoxy)phenyl]acrylate (1.20 g) in tetrahydrofuran (3 ml) and ethanol (3 ml) was added a 1N aqueous sodium hydroxide solution (6.0 ml), and the mixture was stirred at 80° C. for 30 min. A 1N aqueous sodium hydroxide solution (1.0 ml) was added to the reaction mixture, and the mixture was further stirred for 30 min. After allowing to cool to room temperature, 1N hydrochloric acid (7.0 ml) was added to the reaction mixture, and diluted with ethyl acetate. The organic layer was washed with water and saturated brine, dried (MgSO₄), filtrated and concentrated. The obtained residue was recrystallized from ethyl acetate-hexane to give a white solid. Recrystallization from ethyl acetate-hexane gave (2E)-3-[2-{[3-chloro-5-(trifluoromethyl)pyridin-2-yl]oxy}-4-(3-hydroxy-3-methylbutoxy)phenyl]acrylic acid (957 mg, yield: 85%) as white crystals. melting point 192-194° C.

### Reference Example 267

To a solution of (2E)-3-[2-{[3-chloro-5-(trifluoromethyl)pyridin-2-yl]oxy}-4-(3-hydroxy-1,1-dimethylpropoxy)phenyl]acrylate (0.71 g) in acetonitrile (6 ml) were added diisopropylethylamine (384 μl) and chloromethyl methyl ether (1701) at room temperature. Then, diisopropylethylamine (384 μl) and chloromethyl methyl ether (170 μl) were added 4 times in total every one hour, and the mixture was further stirred at room temperature for 1 hr. Water was added to the reaction mixture and diluted with ethyl acetate. The organic layer was washed with water, 1N hydrochloric acid and saturated brine, dried (MgSO₄), filtrated and concentrated. The obtained residue was subjected to silica gel column chromatography, and eluted with ethyl acetate-hexane (hexane alone to 2:3, v/v) to give ethyl (2E)-3-{2-{[3-chloro-5-(trifluoromethyl)pyridin-2-yl]oxy}-4-[3-(methoxymethoxy)-1,1-dimethylpropoxy]phenyl}acrylate (977 mg, quant.) as a colorless oil.

¹H-NMR (300 MHz, CDCl₃) δ:1.29 (3H, t, J=7.8 Hz), 1.38 (6H, s), 1.96-2.07 (2H, m), 3.35 (3H, s), 3.74 (2H, t, J=7.2 Hz), 4.20 (2H, q, J=7.1 Hz), 4.62 (2H, s), 6.40 (1H, d, J=16.0 Hz), 6.78 (1H, d, J=2.3 Hz), 6.93 (1H, dd, J=8.7, 2.3 Hz), 7.60 (1H, d, J=8.7 Hz), 7.72 (1H, d, J=16.0 Hz), 8.01 (1H, d, J=2.1 Hz), 8.23 (1H, d, J=0.8 Hz).

### Reference Example 268

To a solution of ethyl (2E)-3-(2-{[3-chloro-5-(trifluoromethyl)pyridin-2-yl]oxy}-4-hydroxyphenyl)acrylate (5.06 g) in N,N-dimethylformamide (26 ml) were added potassium carbonate (2.71 g), sodium iodide (5.87 g) and isobutylene oxide (50 ml), and the mixture was stirred overnight at 80° C. After allowing to cool to room temperature, 1N hydrochloric acid was added to the reaction mixture, and the mixture was extracted with ethyl acetate. The organic layer was washed with water and saturated brine, dried (MgSO₄), filtrated and concentrated. The obtained residue was subjected to basic silica gel column chromatography, and eluted with ethyl acetate-hexane (hexane alone to 1:1, v/v) to give ethyl (2E)-3-[2-{[3-chloro-5-(trifluoromethyl)pyridin-2-yl]oxy}-4-(2-hydroxy-2-methylpropoxy)phenyl]acrylate (4.67 g, yield: 78%) as a white solid. Recrystallization from ethyl acetate-hexane gave white crystals. melting point 79-83° C.

### Reference Example 269

To a solution of ethyl (2E)-3-[2-{[3-chloro-5-(trifluoromethyl)pyridin-2-yl]oxy}-4-(2-hydroxy-2-methylpropoxy)phenyl]acrylate (1.77 g) in tetrahydrofuran (6 ml) and ethanol (6 ml) was added a 1N aqueous sodium hydroxide solution (9 ml), and the mixture was stirred at 50° C. for 1 hr. After allowing to cool to room temperature, 1N hydrochloric acid (9 ml) was added to the reaction mixture, and the mixture was extracted with ethyl acetate. The organic layer was washed with water and saturated brine, dried (MgSO₄), filtrated and concentrated. The obtained residue was recrystallized from ethyl acetate-hexane to give a white solid. Recrystallization from ethyl acetate-hexane gave (2E)-3-[2-{[3-chloro-5-(trifluoromethyl)pyridin-2-yl]oxy}-4-(2-hydroxy-2-methylpropoxy)phenyl]acrylic acid (1.23 g, yield: 74%) as white crystals. melting point 126.5-128.0° C.

### Reference Example 270

To a solution of ethyl (2E)-3-(2-{[3-chloro-5-(trifluoromethyl)pyridin-2-yl]oxy}-4-hydroxyphenyl)acrylate (5.10 g) in N,N-dimethylformamide (26 ml) were added potassium carbonate (2.75 g), sodium iodide (5.90 g) and 1-bromo-3-methoxypropane (2.61 g), and the mixture was stirred at 50° C. for 1 hr. After allowing to cool to room temperature, 1N hydrochloric acid was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:19-2:3, v/v) to give a white solid. Recrystallization from ethyl acetate-hexane gave ethyl (2E)-3-[2-{[3-chloro-5-(trifluoromethyl)pyridin-2-yl]oxy}-4-(3-methoxypropoxy)phenyl]acrylate (4.22 g, yield: 70%) as white crystals. melting point 86.4-86.5° C.

### Reference Example 271

To a solution of ethyl (2E)-3-[2-{[3-chloro-5-(trifluoromethyl)pyridin-2-yl]oxy}-4-(3-methoxypropoxy)phenyl]acrylate (2.38 g) in tetrahydrofuran (6 ml) and ethanol (6 ml) was added a 1N aqueous sodium hydroxide solution (12 ml), and the mixture was stirred at 50° C. for 30 min. After allowing to cool to room temperature, 1N hydrochloric acid (12 ml) was added to the reaction mixture, and the mixture was extracted with ethyl acetate. The organic layer was washed with water and saturated brine, dried (MgSO₄), filtrated and concentrated. The obtained residue was recrystallized from ethyl acetate-hexane to give a white solid. Recrystallization from ethyl acetate-hexane gave (2E)-3-[2-{[3-chloro-5-(trifluoromethyl)pyridin-2-yl]oxy}-4-(3-methoxypropoxy)phenyl]acrylic acid (1.47 g, yield: 76%) as white crystals. melting point 131-133° C.

### Reference Example 272

To a solution of [2-{[3-chloro-5-(trifluoromethyl)pyridin-2-yl]oxy}-4-(2-methoxyethoxy)phenyl]methanol (11.5 g) in pyridine (25 ml) was added acetic anhydride (25 ml), and the mixture was stirred at room temperature for 1 hr. A saturated aqueous sodium hydrogencarbonate solution was added to the reaction mixture, and the mixture was extracted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (hexane alone to 2:3, v/v) to give 2-{[3-chloro-5-(trifluoromethyl)pyridin-2-yl]oxy}-4-(2-methoxyethoxy)benzyl acetate (8.80 g, yield: 76%) as a white solid. Recrystallization from ethyl acetate-hexane gave white crystals. melting point 52.5-53.0° C.

### Reference Example 273

To a solution of 2-{[3-chloro-5-(trifluoromethyl)pyridin-2-yl]oxy}-4-(2-methoxyethoxy)benzyl acetate (1.80 g) in toluene (20 ml) were added dimethylketene methyl trimethylsilyl acetal (3.50 ml) and magnesium perchlorate (1.44 g), and the mixture was stirred at 50° C. for 3 hr. Water was added to the reaction mixture, and the mixture was diluted with ethyl acetate. The organic layer was washed with saturated brine, dried (MgSO₄), filtrated and concentrated. The obtained residue was subjected to silica gel column chromatography, and eluted with ethyl acetate-hexane (hexane alone to 3:7, v/v) to give methyl 3-[2-{[3-chloro-5-(trifluoromethyl)pyridin-2-yl]oxy}-4-(2-methoxyethoxy)phenyl]-2,2-dimethylpropanoate (1.78 g, yield: 90%) as a colorless oil.

¹H-NMR (300 MHz, CDCl₃) δ:1.17 (6H, s), 2.73 (2H, s), 3.43 (3H, s), 3.66 (3H, s), 3.73 (2H, dd, J=5.5, 4.0 Hz), 4.08 (2H, dd, J=5.5, 4.0 Hz), 6.66 (1H, d, J=2.4 Hz), 6.80 (1H, dd, J 8.6, 2.5 Hz), 7.15-7.24 (1H, m), 7.98 (1H, d, J=2.1 Hz), 8.25 (1H, dd, J 2.3, 0.9 Hz).

### Reference Example 274

To a solution of methyl 3-[2-{[3-chloro-5-(trifluoromethyl)pyridin-2-yl]oxy}-4-(2-methoxyethoxy)phenyl]-2,2-dimethylpropanoate (1.75 g) in tetrahydrofuran (4 ml) was added concentrated sulfuric acid-acetic acid-water (1:6:6, v/v, 8 ml), and the mixture was stirred at 80° C. for 24 hr. After allowing to cool to room temperature, the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:9-1:1, v/v) to give 3-[2-{[3-chloro-5-(trifluoromethyl)pyridin-2-yl]oxy}-4-(2-methoxyethoxy)phenyl]-2,2-dimethylpropanoic acid (1.36 g, yield: 80%) as a colorless oil.

¹H-NMR (300 MHz, CDCl₃) δ: 1.20 (6H, s), 2.77 (2H, s), 3.43 (3H, s), 3.73 (2H, dd, J=5.0, 3.9 Hz), 4.04-4.12 (2H, m), 6.67 (1H, d, J=1.9 Hz), 6.81 (1H, dd, J=8.6, 1.6 Hz), 7.21 (1H, d, J=8.5 Hz), 7.98 (1H, s), 8.25 (1H, d, J=0.9 Hz).

### Reference Example 275

To a solution of ethyl (2E)-3-(2-{[3-chloro-5-(trifluoromethyl)pyridin-2-yl]oxy}-4-hydroxyphenyl)acrylate (5.17 g) in N,N-dimethylformamide (30 ml) were added potassium carbonate (3.64 g), sodium iodide (3.99 g) and 3-(2-methoxyethoxy)propyl bromide (5.25 g), and the mixture was stirred overnight at 50° C. After allowing to cool to room temperature, a saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:19-3:2, v/v) to give a white solid. Recrystallization from ethyl acetate-hexane gave ethyl (2E)-3-{2-{[3-chloro-5-(trifluoromethyl)pyridin-2-yl]oxy}-4-[3-(2-methoxyethoxy)propoxy]phenyl}acrylate (5.34 g, yield: 80%) as white crystals. melting point 65-67° C.

### Reference Example 276

To a solution of ethyl (2E)-3-{2-{[3-chloro-5-(trifluoromethyl)pyridin-2-yl]oxy}-4-[3-(2-methoxyethoxy)propoxy]phenyl}acrylate (2.92 g) in tetrahydrofuran (10 ml) and ethanol (10 ml) was added a 1N aqueous sodium hydroxide solution (13 ml), and the mixture was stirred at 50° C. for 20 min. After allowing to cool to room temperature, 1N hydrochloric acid (13 ml) was added to the reaction mixture, and the mixture was extracted with ethyl acetate. The organic layer was washed with water and saturated brine, dried (MgSO₄), filtrated and concentrated to give a white solid. Recrystallization from ethyl acetate-hexane gave (2E)-3-{2-{[3-chloro-5-(trifluoromethyl)pyridin-2-yl]oxy}-4-[3-(2-methoxyethoxy)propoxy]phenyl}acrylic acid (2.56 g, yield: 93%) as white crystals. melting point 103.0-104.0° C.

### Reference Example 277

To a solution of ethyl (2E)-3-(2-{[3-chloro-5-(trifluoromethyl)pyridin-2-yl]oxy}-4-hydroxyphenyl)acrylate (5.12 g) in N,N-dimethylformamide (30 ml) were added potassium carbonate (3.66 g), sodium iodide (3.89 g) and 1-bromo-2-(2-methoxyethoxy)ethane (3.60 ml), and the mixture was stirred overnight at 50° C. Then, potassium carbonate (7.21 g) and 1-bromo-2-(2-methoxyethoxy)ethane (7.20 ml) were added, and the mixture was further stirred for 4 hr. After allowing to cool to room temperature, a saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:19-3:2, v/v) to give a white solid. Recrystallization from ethyl acetate-hexane gave ethyl (2E)-3-{2-{[3-chloro-5-(trifluoromethyl)pyridin-2-yl]oxy}-4-[2-(2-methoxyethoxy)ethoxy]phenyl}acrylate (5.57 g, yield: 86%) as white crystals. melting point 76.6-76.8° C.

### Reference Example 278

To a solution of ethyl (2E)-3-{2-{[3-chloro-5-(trifluoromethyl)pyridin-2-yl]oxy}-4-[2-(2-methoxyethoxy)ethoxy]phenyl}acrylate (3.32 g) in tetrahydrofuran (10 ml) and ethanol (10 ml) was added a 1N aqueous sodium hydroxide solution (15 ml), and the mixture was stirred at 50° C. for 20 min. After allowing to cool to room temperature, 1N hydrochloric acid (15 ml) was added to the reaction mixture, and the mixture was extracted with ethyl acetate. The organic layer was washed with water and saturated brine, dried (MgSO₄), filtrated and concentrated to give a white solid. Recrystallization from ethyl acetate-hexane gave (2E)-3-{2-{[3-chloro-5-(trifluoromethyl)pyridin-2-yl]oxy}-4-[2-(2-methoxyethoxy)ethoxy]phenyl}acrylic acid (2.01 g, yield: 64%) as white crystals. melting point 133.9-134.0° C.

### Reference Example 279

To a solution of (2E)-3-[2-{[3-chloro-5-(trifluoromethyl)pyridin-2-yl]oxy}-4-(3-methoxypropoxy)phenyl]acrylic acid (1.33 g) in tetrahydrofuran (40 ml) was added a palladium-activated carbon ethylenediamine complex (0.20 g), and the mixture was stirred under a hydrogen atmosphere at room temperature for 4 hr. The reaction mixture was filtrated, a palladium-activated carbon ethylenediamine complex (0.41 g) was added to the filtrate, and the mixture was stirred under a hydrogen atmosphere at room temperature for 2 hr. Methanol (40 ml) was added to the reaction mixture, and the mixture was stirred under a hydrogen atmosphere at room temperature for 1 hr. The reaction mixture was filtrated, and the filtrate was concentrated. The obtained residue was subjected to silica gel column chromatography, and eluted with ethyl acetate-hexane (1:9-1:1, v/v) to give a white solid. Recrystallization from ethyl acetate-hexane gave 3-[2-{[3-chloro-5-(trifluoromethyl)pyridin-2-yl]oxy}-4-(3-methoxypropoxy)phenyl]propanoic acid (571 mg, yield: 43%) as white crystals. melting point 99.1-99.2° C.

### Reference Example 280

To a solution of 2,4-dihydroxybenzaldehyde (24.5 g) in acetonitrile (200 ml) were added potassium bicarbonate (35.5 g) and 3-bromo-1-propanol (49.3 g), and the mixture was stirred at 80° C. for 3 days. After allowing to cool to room temperature, water was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:9-1:1, v/v) to give 2-hydroxy-4-(3-hydroxypropoxy)benzaldehyde (14.2 g, yield: 41%) as a yellow oil.

¹H-NMR (300 MHz, CDCl₃) δ:2.01-2.12 (2H, m), 3.87 (2H, q, J=5.7 Hz), 4.18 (2H, t, J=6.1 Hz), 6.44 (1H, d, J=2.4 Hz), 6.54 (1H, dd, J=8.7, 2.3 Hz), 7.43 (1H, d, J=8.7 Hz), 9.72 (1H, s), 11.47 (1H, s).

### Reference Example 281

To a solution of 2-hydroxy-4-(3-hydroxypropoxy)benzaldehyde (6.90 g) in acetone (170 ml) was added potassium carbonate (5.83 g) and chloromethyl methyl ether (3.20 ml) under ice-cooling, and the mixture was stirred overnight while allowing to warm to room temperature. Then, potassium carbonate (5.83 g) and chloromethyl methyl ether (3.20 ml) were added, and the mixture was stirred at room temperature for 2 hr. Water was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:9-3:2, v/v) to give 4-(3-hydroxypropoxy)-2-(methoxymethoxy)benzaldehyde (4.67 g, yield: 55%) as a yellow oil.

¹H-NMR (300 MHz, CDCl₃) δ:1.53-1.62 (1H, m), 1.97-2.12 (2H, m), 3.53 (3H, s), 3.87 (2H, q, J=5.7 Hz), 4.19 (2H, t, J=6.0 Hz), 5.29 (2H, s), 6.62 (1H, dd, J=8.4, 2.0 Hz), 6.72 (1H, d, J=2.3 Hz), 7.81 (1H, d, J=8.9 Hz), 10.33 (1H, s).

### Reference Example 282

To a solution of 4-(3-hydroxypropoxy)-2-(methoxymethoxy)benzaldehyde (2.02 g) in N,N-dimethylformamide (40 ml) was added sodium hydride (60% in oil, 404 mg) with stirring under ice-cooling, and bromomethylcyclopropane (1.70 ml) was added 30 min later, and the mixture was stirred overnight at room temperature. Then, sodium hydride (60% in oil, 404 mg) and bromomethylcyclopropane (1.70 ml) was added at room temperature, and the mixture was stirred at room temperature for 3 hr and at 50° C. for 2 hr. After allowing to cool to room temperature, water was added to the reaction mixture, and the mixture was diluted with ethyl acetate. The organic layer was washed with water and saturated brine, dried (MgSO₄), filtrated and concentrated. The obtained residue was subjected to silica gel column chromatography, and eluted with ethyl acetate-hexane (hexane alone to 2:3, v/v) to give 4-[3-(cyclopropylmethoxy)propoxy]-2-(methoxymethoxy)benzaldehyde (1.16 g, yield: 47%) as a pale-yellow oil.

¹H-NMR (300 MHz, CDCl₃) δ:0.15-0.23 (2H, m), 0.46-0.63 (2H, m), 0.87-1.14 (1H, m), 2.01-2.16 (2H, m), 3.28 (2H, d, J=6.8 Hz), 3.53 (3H, s), 3.62 (2H, t, J=6.2 Hz), 4.10-4.16 (2H, m), 5.28 (2H, s), 6.53-6.65 (1H, m), 6.70 (1H, d, J=2.3 Hz), 7.81 (1H, d, J=8.7 Hz), 10.32 (1H, s).

### Reference Example 283

To a solution of triethyl phosphonoacetate (1.06 g) in tetrahydrofuran (5 ml) was added sodium hydride (60% in oil, 189 mg) under ice-cooling, a solution of 4-[3-(cyclopropylmethoxy)propoxy]-2-(methoxymethoxy)benzaldehyde (1.16 g) in N,N-dimethylformamide (5 ml) was added dropwise 30 min later, and the mixture was stirred for 20 min while allowing to warm to room temperature. Then, triethyl phosphonoacetate (1.06 g) and sodium hydride (60% in oil, 189 mg) were added at room temperature, and the mixture was further stirred for 20 min. A saturated aqueous ammonium chloride solution was added to the reaction mixture, and the mixture was extracted with ethyl acetate. The organic layer was washed with 1N hydrochloric acid and saturated brine, dried (MgSO₄), filtrated and concentrated to give a yellow oil.

To a solution of the obtained oil in acetone (8 ml) was added 1N hydrochloric acid (4 ml), and the mixture was stirred with heating under reflux for 12 hr. Then, 1N hydrochloric acid (4 ml) was added, and the mixture was further stirred for 3 hr. After allowing to cool to room temperature, a 1N aqueous sodium hydroxide solution (8 ml) was added to the reaction mixture, and the mixture was extracted with ethyl acetate. The organic layer was washed with water and saturated brine, dried (MgSO₄), filtrated and concentrated to give a yellow oil.

To a solution of the obtained oil in N,N-dimethylformamide (15 ml) were added potassium carbonate (1.10 g) and 2,3-dichloro-5-(trifluoromethyl)pyridine (1.10 ml) at room temperature, and the mixture was stirred overnight at 50° C. After allowing to cool to room temperature, a saturated aqueous ammonium chloride solution was added to the reaction mixture, and the mixture was extracted with ethyl acetate. The organic layer was washed with 1N hydrochloric acid and saturated brine, dried (MgSO₄), filtrated and concentrated. The obtained residue was subjected to silica gel column chromatography, and eluted with ethyl acetate-hexane (hexane alone to 2:3, v/v) to give ethyl (2E)-3-{2-{[3-chloro-5-(trifluoromethyl)pyridin-2-yl]oxy}-4-[3-(cyclopropylmethoxy)propoxy]phenyl}acrylate (0.66 g, yield: 34%) as a colorless oil.

¹H-NMR (300 MHz, CDCl₃) δ:0.14-0.23 (2H, m), 0, 45-0.57 (2H, m), 0.88-1.11 (1H, m), 1.28 (3H, t, J=7.1 Hz), 2.06 (2H, tt, J=6.2, 6.2 Hz), 3.26 (2H, d, J=6.8 Hz), 3.60 (2H, t, J=6.1 Hz), 4.09 (2H, t, J=6.0 Hz), 4.20 (2H, q, J=7.2 Hz), 6.36 (1H, d, J=16.0 Hz), 6.68 (1H, d, J=2.4 Hz), 6.87 (1H, dd, J=8.6, 2.4 Hz), 7.63 (1H, d, J=8.9 Hz), 7.69 (1H, d, J=16.0 Hz), 8.01 (1H, d, J=1.7 Hz), 8.25 (1H, dd, J=2.3, 0.9 Hz).

### Reference Example 284

To a solution of methyl 2,4-dihydroxy-3-methylbenzoate (14.8 g) in acetone (200 ml) were added potassium carbonate (22.4 g) and chloromethyl methyl ether (9.0 ml), and the mixture was stirred under ice-cooling for 2 hr. The reaction mixture was filtrated, and the filtrate was concentrated. The obtained residue was subjected to silica gel column chromatography, and eluted with ethyl acetate-hexane (hexane alone to 1:1, v/v) to give a white solid. Recrystallization from ethyl acetate-hexane gave methyl 2-hydroxy-4-(methoxymethoxy)-3-methylbenzoate (11.9 g, yield: 65%) as white crystals. melting point 73.8-74.0° C.

### Reference Example 285

To a solution of methyl 2-hydroxy-4-(methoxymethoxy)-3-methylbenzoate (1.97 g) in N,N-dimethylformamide (16 ml) were added potassium carbonate (2.41 g) and 2,4-dichlorobenzyl chloride (1.50 ml), and the mixture was stirred overnight at 50° C. Water was added to the reaction mixture, and the mixture was diluted with ethyl acetate. The organic layer was washed with water and saturated brine, dried (MgSO₄), filtrated and concentrated to give a white solid. Recrystallization from hexane gave methyl 2-[(2,4-dichlorobenzyl)oxy]-4-(methoxymethoxy)-3-methylbenzoate (3.01 g, yield: 90%) as white crystals. melting point 98.8-99.0° C. A crude product was obtained from the mother liquor (0.73 g).

### Reference Example 286

To a solution of methyl 2-[(2,4-dichlorobenzyl)oxy]-4-(methoxymethoxy)-3-methylbenzoate (2.91 g) in tetrahydrofuran (8 ml) and methanol (8 ml) was added a 1N aqueous sodium hydroxide solution (16 ml), and the mixture was stirred overnight at 50° C. After allowing to cool to room temperature, water was added to the reaction mixture, and the mixture was extracted with ethyl acetate. The organic layer was washed with water and saturated brine, dried (MgSO₄), filtrated and concentrated to give a white solid. Recrystallization from ethyl acetate-hexane gave 2-[(2,4-dichlorobenzyl)oxy]-4-(methoxymethoxy)-3-methylbenzoic acid (2.67 g, yield: 95%) as white crystals. melting point 173.5-174.0° C.

To a solution of a crude product of methyl 2-[(2,4-dichlorobenzyl)oxy]-4-(methoxymethoxy)-3-methylbenzoate

(Reference Example 285, 0.73 g) in tetrahydrofuran (2 ml) and methanol (2 ml) was added a 1N aqueous sodium hydroxide solution (4 ml), and the mixture was stirred overnight at 50° C. After allowing to cool to room temperature, water was added to the reaction mixture, and the mixture was extracted with ethyl acetate. The organic layer was washed with water and saturated brine, dried (MgSO₄), filtrated and concentrated to give a pale-yellow solid. Recrystallization from ethyl acetate-hexane gave a crude product (221 mg, yield: 31%) of 2-[(2,4-dichlorobenzyl)oxy]-4-(methoxymethoxy)-3-methylbenzoic acid as white crystals.

### Reference Example 287

To a solution of 2-[(2,4-dichlorobenzyl)oxy]-4-(methoxymethoxy)-3-methylbenzoic acid (2.59 g) in N,N-dimethylformamide (60 ml) were added 1-ethyl-3-(3-dimethylaminopropyl)carbodiimide hydrochloride (2.01 g), triethylamine (2.90 ml), N,O-dimethylhydroxylamine hydrochloride (1.11 g), and 1-hydroxybenzotriazole hydrate (612 mg), and the mixture was stirred overnight at room temperature. 1N Hydrochloric acid was added to the reaction mixture, and the mixture was extracted with ethyl acetate. The organic layer was washed with 1N hydrochloric acid, a saturated aqueous sodium hydrogencarbonate solution and saturated brine, dried (MgSO₄), filtrated and concentrated to give a crude product as a yellow oil.

To a solution of a crude product of 2-[(2,4-dichlorobenzyl)oxy]-4-(methoxymethoxy)-3-methylbenzoic acid (Reference Example 57, 221 mg) in N,N-dimethylformamide (5 ml) were added 1-ethyl-3-(3-dimethylaminopropyl)carbodiimide hydrochloride (170 mg), triethylamine (0.25 ml), N,O-dimethylhydroxylamine hydrochloride (112 mg), and 1-hydroxybenzotriazole hydrate (137 mg), and the mixture was stirred overnight at room temperature. 1N Hydrochloric acid was added to the reaction mixture, and the mixture was extracted with ethyl acetate. The organic layer was washed with 1N hydrochloric acid, a saturated aqueous sodium hydrogencarbonate solution and saturated brine, dried (MgSO₄), filtrated and concentrated to give a crude product as a yellow oil.

The obtained crude products were combined, subjected to silica gel column chromatography, and eluted with ethyl acetate-hexane (15:85-1:1, v/v) to give 2-[(2,4-dichlorobenzyl)oxy]-N-methoxy-4-(methoxymethoxy)-N,3-dimethylbenzamide (3.04 g, yield: 97%) as a colorless oil.

¹H-NMR (300 MHz, CDCl₃): 2.18 (3H, s), 3.23 (3H, s), 3.50 (3H, s), 3.57 (3H, br. s.), 5.00 (2H, s), 5.23 (2H, s), 6.92 (1H, d, J=8.5 Hz), 7.15 (1H, d, J=8.5 Hz), 7.25-7.31 (1H, m), 7.40 (1H, d, J=2.1 Hz), 7.58 (1H, d, J=8.3 Hz).

### Reference Example 288

To a solution of 2-[(2,4-dichlorobenzyl)oxy]-N-methoxy-4-(methoxymethoxy)-N,3-dimethylbenzamide (3.04 g) in tetrahydrofuran (35 ml) was added a 1.5 M diisobutylaluminum hydride solution in toluene (7.5 ml) under ice-cooling, and the mixture was stirred for 1 hr. Then, under ice-cooling, a 1.5 M diisobutylaluminum hydride solution in toluene (7.5 ml) was added, and the mixture was stirred overnight while allowing to warm to room temperature. Under ice-cooling, a saturated aqueous ammonium chloride solution (4.3 ml) was added dropwise to the reaction mixture, and the mixture was stirred for 1 hr. The mixture was filtered through celite, and the filtrate was concentrated. The obtained residue was subjected to silica gel column chromatography, and eluted with ethyl acetate-hexane (hexane alone to 3:7, v/v) to give 2-[(2,4-dichlorobenzyl)oxy]-4-(methoxymethoxy)-3-methylbenzaldehyde (1.61 g, yield: 62%) as a white solid. Recrystallization from ethyl acetate-hexane gave white crystals. melting point 128.4-128.5° C.

### Reference Example 289

To a solution of triethyl phosphonoacetate (1.06 g) in tetrahydrofuran (5 ml) was added sodium hydride (60% in oil, 189 mg) under ice-cooling, and 30 min later, a solution of 2-[(2,4-dichlorobenzyl)oxy]-4-(methoxymethoxy)-3-methylbenzaldehyde (1.40 g) in N,N-dimethylformamide (5 ml) was added dropwise, and the mixture was stirred for 10 min while allowing to warm to room temperature. Water was added to the reaction mixture, and the mixture was extracted with ethyl acetate. The organic layer was washed with water and saturated brine, dried (MgSO₄), filtrated and concentrated to give ethyl (2E)-3-[2-[(2,4-dichlorobenzyl)oxy]-4-(methoxymethoxy)-3-methylphenyl]acrylate (1.79 g, quant.) as a white solid. Recrystallization from ethyl acetate-hexane gave white crystals. melting point 110-112° C.

### Reference Example 290

To a solution of ethyl (2E)-3-[2-[(2,4-dichlorobenzyl)oxy]-4-(methoxymethoxy)-3-methylphenyl]acrylate (1.79 g) in acetone (16 ml) was added 1N hydrochloric acid (8 ml), and the mixture was stirred overnight with heating under reflux. After allowing to cool to room temperature, a 1N aqueous sodium hydroxide solution (8 ml) was added to the reaction mixture. The resulting solid was collected by filtration and washed with cold water to give ethyl (2E)-3-{2-[(2,4-dichlorobenzyl)oxy]-4-hydroxy-3-methylphenyl}acrylate (0.43 g, yield: 29%) as a white solid.

¹H-NMR (300 MHz, CDCl₃) δ:1.31 (3H, t, J=7.2 Hz), 2.19 (3H, s), 4.23 (2H, q, J=7.2 Hz), 4.88 (2H, s), 5.16 (1H, br. s.), 6.31 (1H, d, J=16.0 Hz), 6.66 (1H, d, J=8.5 Hz), 7.32 (1H, dd, J=8.3, 2.1 Hz), 7.36 (1H, d, J=8.7 Hz), 7.44 (1H, d, J=2.1 Hz), 7.59 (1H, d, J=8.3 Hz), 7.91 (1H, d, J=16.2 Hz).

### Reference Example 291

To a solution of ethyl (2E)-3-{2-[(2,4-dichlorobenzyl)oxy]-4-hydroxy-3-methylphenyl}acrylate (0.43 g) in N,N-dimethylformamide (10 ml) were added potassium carbonate (0.47 g), sodium iodide (0.51 g) and 2-bromoethyl methyl ether (0.47 g), and the mixture was stirred overnight at 50° C. and at 80° C. for 2 hr. Then, potassium carbonate (0.47 g), sodium iodide (0.51 g) and 2-bromoethyl methyl ether (0.47 g) were added, and the mixture was further stirred for 2 hr. After allowing to cool to room temperature, water was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (hexane alone to 3:7, v/v) to give ethyl (2E)-3-[2-[(2,4-dichlorobenzyl)oxy]-4-(2-methoxyethoxy)-3-methylphenyl]acrylate (180 mg, yield: 36%) as a white solid. Recrystallization from ethyl acetate-hexane gave white crystals. melting point 92.0-92.5° C.

### Reference Example 292

To a solution of ethyl (2E)-3-[2-[(2,4-dichlorobenzyl)oxy]-4-(2-methoxyethoxy)-3-methylphenyl]acrylate (170 mg) in tetrahydrofuran (2 ml) and ethanol (2 ml) was added a 1N aqueous sodium hydroxide solution (6.0 ml), and the mixture was stirred overnight at 80° C. After allowing to cool to room temperature, 1N hydrochloric acid (6.0 ml) was added to the reaction mixture, and the mixture was extracted with ethyl acetate. The organic layer was washed with saturated brine, dried (MgSO₄), filtrated and concentrated to give (2E)-3-[2-[(2,4-dichlorobenzyl)oxy]-4-(2-methoxyethoxy)-3-methylphenyl]acrylic acid (99 mg, yield: 24%) as a white solid. Recrystallization from ethyl acetate-hexane gave white crystals.

¹H-NMR (300 MHz, DMSO-d₆) δ:2.1Q (3H, s), 3.30 (3H, s), 3.58-3.75 (2H, m), 4.12-4.21 (2H, m), 4.84 (2H, s), 6.36 (1H, d, J=16.0 Hz), 6.88 (1H, d, J=8.7 Hz), 7.46-7.53 (1H, m), 7.62 (2H, d, J=8.3 Hz), 7.69 (1H, d, J=2.1 Hz), 7.74 (1H, d, J=16.0 Hz).

### Reference Example 293

To a solution of ethyl (2E)-3-(2-{[3-chloro-5-(trifluoromethyl)pyridin-2-yl]oxy}-4-hydroxyphenyl)acrylate (2.53 g) in tetrahydrofuran (100 ml) were added tributylphosphine (3.30 ml), 2-(cyclopropyloxy)ethanol (0.80 g) and 1,1′-(azodicarbonyl)dipiperidine (2.47 g), and the mixture was stirred at 50° C. for 30 min. The reaction mixture was concentrated, the obtained solid was washed with diisopropyl ether, and the filtrate was concentrated. The obtained residue was subjected to silica gel column chromatography, and eluted with ethyl acetate-hexane (hexane alone to 35:65, v/v) to give ethyl (2E)-3-{2-{[3-chloro-5-(trifluoromethyl)pyridin-2-yl]oxy}-4-[2-(cyclopropyloxy)ethoxy]phenyl}acrylate (2.21 g, yield: 72%) as a white solid. Recrystallization from ethyl acetate-hexane gave white crystals. melting point 75.4-75.9° C.

### Reference Example 294

To a solution of ethyl (2E)-3-{2-{[3-chloro-5-(trifluoromethyl)pyridin-2-yl]oxy}-4-[2-(cyclopropyloxy)ethoxy]phenyl}acrylate (2.01 g) in tetrahydrofuran (10 ml) and ethanol (10 ml) was added a 1N aqueous sodium hydroxide solution (10 ml), and the mixture was stirred at room temperature for 4 hr. 1N Hydrochloric acid (10 ml) was added to the reaction mixture, and diluted with ethyl acetate. The organic layer was washed with water and saturated brine, dried (MgSO₄), filtrated and concentrated to give (2E)-3-{2-{[3-chloro-5-(trifluoromethyl)pyridin-2-yl]oxy}-4-[2-(cyclopropyloxy)ethoxy]phenyl}acrylic acid (1.83 g, yield: 97%) as a white solid. Recrystallization from ethyl acetate-hexane gave white crystals. melting point 147-150° C.

### Reference Example 295

To a solution of ethyl (2E)-3-(2-{[3-chloro-5-(trifluoromethyl)pyridin-2-yl]oxy}-4-hydroxyphenyl)acrylate (5.12 g) in tetrahydrofuran (40 ml) was added a palladium-activated carbon ethylenediamine complex (0.23 g), and the mixture was stirred under a hydrogen atmosphere at room temperature for 4 hr. The reaction mixture was filtrated, a palladium-activated carbon ethylenediamine complex (0.50 g) was added to the filtrate, and the mixture was stirred under a hydrogen atmosphere at room temperature for 3 hr. Then, methanol (40 ml) was added, and the mixture was further stirred under a hydrogen atmosphere at room temperature for 15 hr. The reaction mixture was filtrated, and the filtrate was concentrated. The obtained residue was subjected to silica gel column chromatography, and eluted with ethyl acetate-hexane (1:19-1:1, v/v) to give an orange oil.

To a solution of the obtained oil in N,N-dimethylformamide

(30 ml) were added potassium carbonate (1.79 g), sodium iodide (1.95 g) and 2-(2-bromoethyl)-2-methyl-1,3-dioxolane (1.78 ml), and the mixture was stirred overnight at 60° C. After allowing to cool to room temperature, water was added to the reaction mixture, and the mixture was extracted with ethyl acetate. The organic layer was washed with water and saturated brine, dried (MgSO₄), filtrated and concentrated. The obtained residue was subjected to basic silica gel column chromatography, and eluted with ethyl acetate-hexane (hexane alone to 2:3, v/v) to give ethyl 3-{2-{[3-chloro-5-(trifluoromethyl)pyridin-2-yl]oxy}-4-[2-(2-methyl-1,3-dioxolan-2-yl)ethoxy]phenyl}propanoate (0.77 g, yield: 19%) as a colorless oil.

¹H-NMR (300 MHz, CDCl₃) δ:1.21 (3H, t, J=7.1 Hz), 1.38 (3H, s), 2.15 (2H, t, J=7.0 Hz), 2.56 (2H, t, J=7.5 Hz), 2.76 (2H, t, J=7.7 Hz), 3.85-4.00 (4H, m), 4.01-4.15 (4H, m), 6.65 (1H, d, J=2.6 Hz), 6.79 (1H, dd, J=8.5, 2.4 Hz), 7.22 (1H, d, J=8.5 Hz), 7.98 (1H, d, J=2.3 Hz), 8.26 (1H, dd, J=2.1, 0.9 Hz).

### Reference Example 296

To a solution of ethyl (2E)-3-[2-hydroxy-4-(2-methoxyethoxy)phenyl]acrylate (875 mg) in N,N-dimethylformamide (8 ml) were added potassium carbonate (695 mg) and 3-chloro-4-nitrobenzotrifluoride (894 mg), and the mixture was stirred at 50° C. for 3 hr. After allowing to cool to room temperature, 1N hydrochloric acid was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (hexane alone to 2:3, v/v) to give a brown solid. Recrystallization from ethyl acetate-hexane gave ethyl (2E)-3-{4-(2-methoxyethoxy)-2-[2-nitro-5-(trifluoromethyl)phenoxy]phenyl}acrylate (0.97 g, yield: 65%) as red-brown crystals. melting point 108-110° C.

### Reference Example 297

To a solution of ethyl 3-[2-hydroxy-4-(2-methoxyethoxy)phenyl]propanoate (8.98 g) in N,N-dimethylformamide (60 ml) were added potassium carbonate (9.25 g) and benzyl bromide (5.0 ml), and the mixture was stirred at room temperature for 3 hr. A saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to basic silica gel column chromatography, and eluted with ethyl acetate-hexane (hexane alone to 35:65, v/v) to give a colorless oil. The oil, was dissolved in ethyl acetate, and the solution was washed with water and saturated brine, dried (MgSO₄), filtrated and concentrated to give ethyl 3-[2-(benzyloxy)-4-(2-methoxyethoxy)phenyl]propanoate (11.5 g, yield: 96%) as a white solid. Recrystallization from ethyl acetate-hexane gave white crystals. melting point 38-39° C.

### Reference Example 298

To a solution of ethyl (2E)-3-[2-{[3-chloro-5-(trifluoromethyl)pyridin-2-yl]oxy}-4-(2-hydroxy-2-methylpropoxy)phenyl]acrylate (2.44 g) in tetrahydrofuran (50 ml) was added 10% palladium-activated carbon (1.06 g), and the mixture was stirred under a hydrogen atmosphere at room temperature for 1 hr. The reaction mixture was filtrated, and the filtrate was concentrated. The obtained residue was subjected to silica gel column chromatography, and eluted with ethyl acetate-hexane (hexane alone to 2:3, v/v) to give a yellow oil.

To a solution of the obtained oil in tetrahydrofuran (30 ml) was added a 1.5 M diisobutylaluminum hydride solution in toluene (12 ml) under ice-cooling, and the mixture was stirred for 20 min. Under ice-cooling, a saturated aqueous ammonium chloride solution was added dropwise to the reaction mixture, and the mixture was stirred for 1 hr. The mixture was filtered through celite, and the filtrate was concentrated. The obtained residue was subjected to silica gel column chromatography, and eluted with ethyl acetate-hexane (hexane alone to 1:1, v/v) to give 3-[2-{[3-chloro-5-(trifluoromethyl)pyridin-2-yl]oxy}-4-(2-hydroxy-2-methylpropoxy)phenyl]propan-1-ol (1.10 g, yield: 45%) as a white solid. Recrystallization from ethyl acetate-hexane gave white crystals. melting point 81.2-82.0° C.

### Reference Example 299

To a solution of ethyl (2E)-3-[2-hydroxy-4-(2-methoxyethoxy)phenyl]acrylate in dichloromethane (100 ml) were added pyridine (25.0 ml) and triflic anhydride (6.0 ml) under ice-cooling, and the mixture was stirred for 5 min. Water was added to the reaction mixture, and the mixture was diluted with ethyl acetate. The organic layer was washed with 1N hydrochloric acid and saturated brine, dried (MgSO₄), filtrated and concentrated. The obtained residue was subjected to basic silica gel column chromatography, and eluted with ethyl acetate-hexane (hexane alone to 35:65, v/v) to give ethyl (2E)-3-(4-(2-methoxyethoxy)-2-{[(trifluoromethyl)sulfonyl]oxy}phenyl)acrylate (12.56 g, quant.) as a white solid. Recrystallization from ethyl acetate-hexane gave white crystals. melting point 37.1-37.3° C.

### Reference Example 300

A mixture of ethyl (2E)-3-[2-[(3,5-dichloropyridin-2-yl)oxy]-4-(2-methoxyethoxy)phenyl]acrylate (2.96 g), a palladium-activated carbon ethylenediamine complex (0.30 g) and methanol (200 ml) was stirred under a hydrogen atmosphere at room temperature for 3 hr. The catalyst was filtered off, and the filtrate was concentrated. The obtained residue was subjected to silica gel column chromatography, and eluted with ethyl acetate-hexane (1:19-2:3, v/v) to give ethyl 3-[2-[(3,5-dichloropyridin-2-yl)oxy]-4-(2-methoxyethoxy)phenyl]propanoate (2.04 g, yield: 69%) as a colorless oil.

¹H-NMR (300 MHz, CDCl₃) δ:1.21 (3H, t, J=7.2 Hz), 2.56 (2H, t, J=7.6 Hz), 2.78 (2H, t, J=7.8 Hz), 3.45 (3H, s), 3.65-3.79 (2H, m), 4.03-4.15 (4H, m), 6.64 (1H, d, J=2.7 Hz), 6.77 (1H, dd, J=8.7, 2.7 Hz), 7.20 (1H, d, J=8.3 Hz), 7.77 (1H, d, J=2.7 Hz), 7.95 (1H, d, J=2.3 Hz).

### Reference Example 301

Ethyl 3-[2-[(3,5-dichloropyridin-2-yl)oxy]-4-(2-methoxyethoxy)phenyl]propanoate (2.04 g) was dissolved in diethyl ether (9.8 ml), a 1.5 M diisobutylaluminum hydride solution in toluene (8.2 ml) was added at 0° C., and the mixture was stirred at room temperature for 2.5 hr. Methanol and water were added to the reaction mixture, the mixture was stirred for a while, filtered through celite, and concentrated. The obtained residue was subjected to silica gel column chromatography, and eluted with ethyl acetate-hexane (3:7-3:2, v/v) to give colorless crystals. Recrystallization from ethyl acetate-hexane gave 3-[2-[(3,5-dichloropyridin-2-yl)oxy]-4-(2-methoxyethoxy)phenyl]propan-1-ol (1.48 g, yield: 81%) as colorless crystals. melting point 83.9-84.5° C.

### Reference Example 302

To a solution of ethyl (2E)-3-{2-{[3-chloro-5-(trifluoromethyl)pyridin-2-yl]oxy}-4-[2-ethoxy-1-(ethoxymethyl)ethoxy]phenyl}acrylate (1.00 g) in tetrahydrofuran (10 ml) was added a 1.5 M diisobutylaluminum hydride solution in toluene (4.97 ml) under ice-cooling over 15 min, and the mixture was stirred for 1 hr. The reaction mixture was stirred at room temperature for 2 hr, sodium sulfate decahydrate (2.40 g) was added under ice-cooling, and the mixture was stirred for 12 hr. The insoluble material was filtered off and the filtrate was concentrated. The residue was subjected to silica gel column chromatography, and eluted with ethyl acetate-hexane (1:20-1:3, v/v) to give a white solid. Recrystallization from diethyl ether-hexane gave (2E)-3-{2-{[3-chloro-5-(trifluoromethyl)pyridin-2-yl]oxy}-4-[2-ethoxy-1-(ethoxymethyl)ethoxy]phenyl}prop-2-en-1-ol (40 mg, yield: 4%) as a white powder. melting point 52.5-54.5° C.

### Reference Example 303

A mixture of (2E)-3-{2-{[3-chloro-5-(trifluoromethyl)pyridin-2-yl]oxy}-4-[2-ethoxy-1-(ethoxymethyl)ethoxy]phenyl}prop-2-en-1-ol (135 mg), 5% palladium-carbon (16 mg) and ethyl acetate (5 ml) was stirred under a hydrogen atmosphere at room temperature for 2.5 hr. Palladium was filtered off, and the filtrate was concentrated. The residue was subjected to silica gel column chromatography, and eluted with ethyl acetate-hexane (1:20-1:3, v/v) to give 3-{2-{[3-chloro-5-(trifluoromethyl)pyridin-2-yl]oxy}-4-[2-ethoxy-1-(ethoxymethyl)ethoxy]phenyl}propan-1-ol (120 mg, yield: 88%) as a pale-yellow oil.

¹H-NMR (300 MHz, CDCl₃) δ:1.17 (6H, t, J=7.0 Hz), 1.75-1.91 (2H, m), 2.46-2.63 (2H, m), 3.45-3.57 (4H, m), 3.57-3.71 (6H, m), 4.38-4.51 (1H, m), 6.74 (1H, d, J=2.5 Hz), 6.89 (1H, dd, J=8.5, 2.5 Hz), 7.21 (1H, d, J=8.5 Hz), 7.98 (1H, dd, J=2.2, 0.5 Hz), 8.25 (1H, dd, J=2.1, 0.9 Hz).

### Reference Example 304

A suspension of ethyl (2E)-3-(4-(2-{[tert-butyl(diphenyl)silyl]oxy}ethoxy)-2-{[3-chloro-5-(trifluoromethyl)pyridin-2-yl]oxy}phenyl)acrylate (2.52 g) and 10% palladium-carbon in ethanol was stirred under a hydrogen atmosphere at room temperature for 4 hr. The reaction mixture was filtrated, and the solvent was evaporated under reduced pressure. The residue was subjected to silica gel column chromatography, and eluted with ethyl acetate-hexane (0:1-2:8, v/v) to give ethyl 3-(4-(2-{[tert-butyl(diphenyl)silyl]oxy}ethoxy)-2-{[3-chloro-5-(trifluoromethyl)pyridin-2-yl]oxy}phenyl)propanoate (1.88 g, yield: 74%) as a colorless oil.

¹H-NMR (300 MHz, CDCl₃) δ:1.04 (9H, s), 1.14-1.32 (4H, m), 2.57 (2H, t, J=7.8 Hz), 2.76 (2H, t, J=7.8 Hz), 3.89-4.19 (6H, m), 6.63 (1H, d, J=2.7 Hz), 6.76 (1H, dd, J 8.5, 2.5 Hz), 7.21 (1H, d, J=8.7 Hz), 7.31-7.47 (6H, m), 7.63-7.77 (4H, m), 7.99 (1H, d, J=2.3 Hz), 8.25 (1H, s).

### Reference Example 305

Under ice-cooling, to a solution of ethyl 3-(4-(2-{[tert-butyl(diphenyl)silyl]oxy}ethoxy)-2-{[3-chloro-5-(trifluoromethyl)pyridin-2-yl]oxy}phenyl)propanoate (1.88 g) in diethyl ether (10 ml) was added dropwise a 1.5 M diisobutylaluminum hydride solution in toluene (5.6 ml), and the mixture was stirred while warming to room temperature over 4 hr. Saturated brine was added to the reaction mixture, the mixture was filtrated, and the filtrate was extracted with ethyl acetate. The ethyl acetate layer was washed with saturated brine, dried (MgSO₄), and concentrated. The residue was subjected to silica gel column chromatography, and eluted with ethyl acetate-hexane (0:1-6:4, v/v) to give 3-(4-(2-{[tert-butyl(diphenyl)silyl]oxy}ethoxy)-2-{[3-chloro-5-(trifluoromethyl)pyridin-2-yl]oxy}phenyl)propan-1-ol (1.06 g, yield: 60%) as a colorless oil.

¹H-NMR (300 MHz, CDCl₃) δ:1.04 (9H, s), 1.36 (1H, t, J=5.7 Hz), 1.75-1.89 (2H, m), 2.53 (2H, t, J=7.5 Hz), 3.61 (2H, q, J=6.1 Hz), 3.93-4.08 (4H, m), 6.62 (1H, d, J=2.6 Hz), 6.78 (1H, dd, J=8.5, 2.6 Hz), 7.21 (1H, d, J=8.5 Hz), 7.31-7.46 (6H, m), 7.64-7.75 (4H, m), 7.99 (1H, d, J=2.3 Hz), 8.25 (1H, d, J=1.1 Hz).

### Reference Example 306

Under ice-cooling, to a solution of 3-(2-{[3-chloro-5-(trifluoromethyl)pyridin-2-yl]oxy}-4-isopropoxyphenyl)propanoic acid (6.00 g) and triethylamine (2.26 g) in tetrahydrofuran (100 ml) was added isobutylchloroformate (2.24 g), and the mixture was stirred for 10 min. At the same temperature, methanol (50 ml), water (50 ml) and sodium borohydride (1.41 g) were added, and the mixture was stirred while warming to room temperature over 1 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-1:1, v/v) to give 3-(2-{[3-chloro-5-(trifluoromethyl)pyridin-2-yl]oxy}-4-isopropoxyphenyl)propan-1-ol (3.86 g, yield: 66%) as a white solid.

¹H-NMR (300 MHz, CDCl₃) δ:1.32 (6H, d, J=6.2 Hz), 1.75-1.90 (2H, m), 2.54 (2H, t, J=7.5 Hz), 3.61 (2H, t, J=6.3 Hz), 4.40-4.56 (1H, m), 6.61 (1H, d, J=2.4 Hz), 6.78 (1H, dd, J=8.5, 2.4 Hz), 7.21 (1H, d, J=8.5 Hz), 7.98 (1H, d, J=1.7 Hz), 8.23-8.30 (1H, m).

### Reference Example 307

Under ice-cooling, to a mixed solution of 2-(benzyloxy)-4-(2-methoxyethoxy)benzaldehyde (5.00 g) and ethyl chloroacetate (1.85 ml) in tetrahydrofuran (60 ml)/tert-butanol (20 ml) was added potassium tert-butoxide (2.14 g), and the mixture was stirred at the same temperature for 1 hr. A 1N aqueous sodium hydroxide solution (20 ml) was added, and the mixture was stirred while warming to room temperature over 2 hr. Acetic acid (10 ml) was added, and the mixture was stirred at 50° C. for 4 hr. The reaction solution was allowed to cool to room temperature, a 1N aqueous sodium hydroxide solution and ethyl acetate were added, and the mixture was washed successively with water and a saturated aqueous sodium hydrogencarbonate solution. The organic layer was dried (MgSO₄), and the solvent was evaporated under reduced pressure. The residue was subjected to silica gel column chromatography, and eluted with ethyl acetate-hexane (0:1-1:1, v/v) to give [2-(benzyloxy)-4-(2-methoxyethoxy)phenyl]acetaldehyde (1.80 g, yield: 34%) as a colorless oil.

¹H-NMR (300 MHz, CDCl₃) δ:3.45 (3H, s), 3.62 (2H, d, J=2.1 Hz), 3.70-3.78 (2H, m), 4.07-4.15 (2H, m), 5.05 (2H, s), 6.50 (1H, dd, J=8.3, 2.4 Hz), 6.62 (1H, d, J=2.3 Hz), 7.05 (1H, d, J=8.3 Hz), 7.28-7.43 (5H, m), 9.68 (1H, t, J=2.1 Hz).

### Reference Example 308

Under ice-cooling, to a mixture of [2-(benzyloxy)-4-(2-methoxyethoxy)phenyl]acetaldehyde (1.80 g), methanol (25 ml) and water (25 ml) was added sodium borohydride (0.34 g) by small portions, and the mixture was stirred at the same temperature for 2 hr. 1N Hydrochloric acid was added to the reaction mixture, methanol was evaporated under reduced pressure, and the residue was extracted with ethyl acetate. The ethyl acetate layer was washed with saturated brine, dried (MgSO₄), and concentrated. The residue was subjected to silica gel column chromatography, and eluted with ethyl acetate-hexane (0:1-7:3, v/v) to give 2-[2-(benzyloxy)-4-(2-methoxyethoxy)phenyl]ethanol (1.50 g, yield: 83%) as a colorless oil.

¹H-NMR (300 MHz, CDCl₃) δ:2.88 (2H, t, J=6.4 Hz), 3.45 (3H, s), 3.70-3.76 (2H, m), 3.81 (2H, t, J=6.3 Hz), 4.06-4.13 (2H, m), 5.04 (2H, s), 6.47 (1H, dd, J=8.3, 2.4 Hz), 6.60 (1H, d, J=2.4 Hz), 7.08 (1H, d, J=8.3 Hz), 7.28-7.48 (5H, m).

### Reference Example 309

Under ice-cooling, to a mixed solution of 2-{[3-chloro-5-(trifluoromethyl)pyridin-2-yl]oxy}-4-(methoxymethoxy)benzaldehyde (10.0 g) and ethyl bromoacetate (11.6 g) in tetrahydrofuran (100 ml)/tert-butanol (100 ml) was added potassium tert-butoxide (7.78 g), and the mixture was stirred at the same temperature for 12 hr. The solvent was evaporated under reduced pressure, 8N aqueous sodium hydroxide solution (17 ml), water (34 ml) and tetrahydrofuran (50 ml) were added to the residue, and the mixture was stirred at room temperature for 2 hr. Acetic acid (200 ml) was added, and the mixture was stirred at 60° C. for 4 hr. The reaction solution was allowed to cool to room temperature, and neutralized with a 8N aqueous sodium hydroxide solution and ethyl acetate was added. The mixture was washed successively with water and a saturated aqueous sodium hydrogencarbonate solution. The organic layer was dried (MgSO₄), and the solvent was evaporated under reduced pressure. The residue was subjected to silica gel column chromatography, and eluted with ethyl acetate-hexane (0:1-4:6, v/v). The obtained solid was recrystallized from ethyl acetate-hexane to give [2-{[3-chloro-5-(trifluoromethyl)pyridin-2-yl]oxy}-4-(methoxymethoxy)phenyl]acetaldehyde (2.07 g, yield: 20%) as colorless crystals.

¹H-NMR (300 MHz, CDCl₃) δ:3.48 (3H, s), 3.54 (2H, d, J=1.9 Hz), 5.18 (2H, s), 6.91 (1H, d, J=2.7 Hz), 7.00 (1H, dd, J=8.5, 2.5 Hz), 7.22-7.28 (1H, m), 7.99 (1H, d, J=1.9 Hz), 8.25 (1H, s), 9.67 (1H, t, J=2.1 Hz).

### Reference Example 310

Under ice-cooling, to a mixture of [2-{[3-chloro-5-(trifluoromethyl)pyridin-2-yl]oxy}-4-(methoxymethoxy)phenyl]acetaldehyde (1.54 g), methanol (60 ml) and water (25 ml) was added sodium borohydride (0.23 g) by small portions, and the mixture was stirred at the same temperature for 2 hr. 1N Hydrochloric acid was added to the reaction mixture, methanol was evaporated under reduced pressure, and the residue was extracted with ethyl acetate. The ethyl acetate layer was washed with saturated brine, dried (MgSO₄), and concentrated. The residue was subjected to silica gel column chromatography, and eluted with ethyl acetate-hexane (0:1-71:1, v/v) to give 2-[2-{[3-chloro-5-(trifluoromethyl)pyridin-2-yl]oxy}-4-(methoxymethoxy)phenyl]ethanol (1.32 g, yield: 85%) as a white solid.

¹H-NMR (300 MHz, CDCl₃) δ:1.49-1.58 (1H, m), 2.75 (2H, d), 3.47 (3H, d), 3.80 (2H, d), 5.15 (2H, d), 6.81 (1H, d, J=2.4 Hz), 6.96 (1H, dd, J=8.5, 2.4 Hz), 7.22-7.32 (1H, m), 7.99 (1H, d, J=2.3 Hz), 8.26 (1H, dd, J=2.1, 0.9 Hz).

### Reference Example 311

To 2-[2-{[3-chloro-5-(trifluoromethyl)pyridin-2-yl]oxy}-4-(methoxymethoxy)phenyl]ethyl {[(2-isopropoxyethyl)amino]sulfonyl}carbamate (0.19 g) was added a 10% hydrochloric acid-methanol solution (5 ml), and the mixture was stirred at 40° C. for 3 hr. A saturated aqueous sodium hydrogencarbonate solution was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-1:1, v/v), concentrated, and crystallized from ethyl acetate-hexane to give 2-(2-{[3-chloro-5-(trifluoromethyl)pyridin-2-yl]oxy}-4-hydroxyphenyl)ethyl {[(2-isopropoxyethyl)amino]sulfonyl}carbamate (0.085 g, yield: 48%) as colorless crystals.

¹H-NMR (300 MHz, CDCl₃) δ:1.15 (6H, d, J=6.0 Hz), 2.87 (2H, t, J=6.7 Hz), 3.13 (2H, q, J=5.7 Hz), 3.44-3.65 (3H, m), 4.36 (2H, t, J=6.6 Hz), 5.21 (1H, s), 5.28-5.41 (1H, m), 6.59 (1H, d, J=2.6 Hz), 6.73 (1H, dd, J=8.4, 2.5 Hz), 7.19 (1H, d, J=8.5 Hz), 7.61 (1H, s), 8.03 (1H, d, J=2.1 Hz), 8.32 (1H, d, J=1.1 Hz).

### Reference Example 312

A suspension of ethyl (2E)-3-[2-{[3-chloro-5-(trifluoromethyl)pyridin-2-yl]oxy}-4-(methoxymethoxy)phenyl]acrylate (9.5 g) and 10% palladium-carbon in ethanol (300 ml) was stirred under a hydrogen atmosphere at room temperature for 4 hr. The reaction mixture was filtrated, and the solvent was evaporated under reduced pressure. The residue was subjected to silica gel column chromatography, and eluted with ethyl acetate-hexane (0:1-2:8, v/v) to give ethyl 3-[2-{[3-chloro-5-(trifluoromethyl)pyridin-2-yl]oxy}-4-(methoxymethoxy)phenyl]propanoate (8.1 g, yield: 85%) as a colorless oil.

¹H-NMR (300 MHz, CDCl₃) δ:1.17-1.30 (3H, t, J=7.2 Hz), 2.52-2.62 (2H, m), 2.77 (2H, t, J=7.6 Hz), 3.47 (3H, s), 4.09 (2H, q, J=7.2 Hz), 5.15 (2H, s), 6.81 (1H, d, J=2.3 Hz), 6.88-6.99 (1H, m), 7.24 (1H, d, J=8.3 Hz), 7.98 (1H, d, J=1.9 Hz), 8.26 (1H, s).

### Reference Example 313

Under ice-cooling, to a solution of ethyl 3-[2-{[3-chloro-5-(trifluoromethyl)pyridin-2-yl]oxy}-4-(methoxymethoxy)phenyl]propanoate (8.0 g) in diethyl ether (30 ml) was added dropwise a 1.5 M diisobutylaluminum hydride solution in toluene (36.8 ml), and the mixture was stirred while warming to room temperature over 4 hr. Saturated brine was added to the reaction mixture, the mixture was filtrated, and the filtrate was extracted with ethyl acetate. The ethyl acetate layer was washed with saturated brine, dried (MgSO₄), and concentrated. The residue was subjected to silica gel column chromatography, and eluted with ethyl acetate-hexane (0:1-1:1, v/v) to give 3-[2-{[3-chloro-5-(trifluoromethyl)pyridin-2-yl]oxy}-4-(methoxymethoxy)phenyl]propan-1-ol (5.18 g, yield: 72%) as a white solid.

¹H-NMR (300 MHz, CDCl₃) δ:1.36 (1H, t, J=5.6 Hz), 1.75-1.91 (2H, m), 2.48-2.60 (2H, m), 3.48 (3H, s), 3.61 (2H, q, J=6.2 Hz), 5.15 (2H, s), 6.80 (1H, d, J=2.4 Hz), 6.94 (1H, dd, J=8.5, 2.4 Hz), 7.24 (1H, d, J=8.7 Hz), 7.98 (1H, d, J=1.9 Hz), 8.26 (1H, dd, J=2.2, 1.0 Hz).

### Reference Example 314

To 3-[2-{[3-chloro-5-(trifluoromethyl)pyridin-2-yl]oxy}-4-(methoxymethoxy)phenyl]propyl {[(2-isopropoxyethyl)amino]sulfonyl}carbamate (5.2 g) was added a 10% hydrochloric acid-methanol solution (50 ml), and the mixture was stirred at 40° C. for 3 hr. A saturated aqueous sodium hydrogencarbonate solution was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-6:4, v/v) to give 3-(2-{[3-chloro-5-(trifluoromethyl)pyridin-2-yl]oxy}-4-hydroxyphenyl)propyl {[(2-isopropoxyethyl)amino]sulfonyl}carbamate (3.9 g, yield: 81%) as a colorless oil.

¹H-NMR (300 MHz, CDCl₃) δ:1.17 (6H, d, J=6.1 Hz), 1.86-2.02 (2H, m), 2.62 (2H, t, J=7.4 Hz), 3.24 (2H, q, J=5.6 Hz), 3.50-3.70 (3H, m), 4.13 (2H, t, J=6.1 Hz), 5.34 (1H, t, J=5.9 Hz), 5.48 (1H, br.s.), 6.64 (1H, d, J=2.3 Hz), 6.71 (1H, dd, J=8.1, 2.5 Hz), 7.14 (1H, d, J=8.3 Hz), 7.92 (1H, br.s.), 8.03 (1H, d, J=2.3 Hz), 8.31 (1H, s).

### Reference Example 315

A mixture of ethyl (2E)-3-(2-{[3-chloro-5-(trifluoromethyl)pyridin-2-yl]oxy}-4-hydroxyphenyl)acrylate (3.00 g), 4-bromobutyronitrile (1.72 g), potassium carbonate (2.14 g), sodium iodide (1.74 g) and N,N-dimethylformamide (20 ml) was stirred at 50° C. for 15 hr. After cooling, the reaction mixture was poured into water, and the mixture was extracted with ethyl acetate. The ethyl acetate layer was washed with saturated brine, dried (MgSO₄), and concentrated. The obtained solid was recrystallized from ethyl acetate-hexane to give ethyl (2E)-3-[2-{[3-chloro-5-(trifluoromethyl)pyridin-2-yl]oxy}-4-(3-cyanopropoxy)phenyl]acrylate (3.30 g, yield: 94%) as colorless crystals. melting point 106-107° C.

### Reference Example 316

A mixture of ethyl (2E)-3-(2-{[3-chloro-5-(trifluoromethyl)pyridin-2-yl]oxy}-4-hydroxyphenyl)acrylate (3.00 g), 2-(2-bromoethyl)-2-methyl-1,3-dioxolane (2.27 g), potassium carbonate (2.14 g), sodium iodide (1.74 g), and N,N-dimethylformamide (20 ml) was stirred at 50° C. for 15 hr and at 80° C. for 6 hr. After cooling, the reaction mixture was poured into water, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:4-1:1, v/v). The obtained solid was recrystallized from ethyl acetate-hexane to give ethyl (2E)-3-{2-{[3-chloro-5-(trifluoromethyl)pyridin-2-yl]oxy}-4-[2-(2-methyl-1,3-dioxolan-2-yl)ethoxy]phenyl}acrylate (2.86 g, yield: 74%) as colorless crystals. melting point 90-91° C.

### Reference Example 317

A mixture of ethyl (2E)-3-(2-{[3-chloro-5-(trifluoromethyl)pyridin-2-yl]oxy}-4-hydroxyphenyl)acrylate (3.00 g), bromoacetone (1.59 g), potassium carbonate (2.14 g), sodium iodide (1.74 g) and N,N-dimethylformamide (20 ml) was stirred at room temperature for 15 hr. The reaction mixture was poured into water, and the mixture was extracted with ethyl acetate. The ethyl acetate layer was washed with saturated brine, dried (MgSO₄), and concentrated. The obtained solid was recrystallized from ethyl acetate-hexane to give ethyl (2E)-3-[2-{[3-chloro-5-(trifluoromethyl)pyridin-2-yl]oxy}-4-(2-oxopropoxy)phenyl]acrylate (2.77 g, yield: 81%) as colorless crystals. melting point 167-168° C.

### Reference Example 318

A mixture of ethyl (2E)-3-(2-{[3-chloro-5-(trifluoromethyl)pyridin-2-yl]oxy}-4-hydroxyphenyl)acrylate (3.00 g), glycidyl isopropyl ether (4.50 g), potassium carbonate (6.42 g), sodium iodide (5.80 g), and N,N-dimethylformamide (20 ml) was stirred at 50° C. for 15 hr and at 80° C. for 6 hr. After cooling, the reaction mixture was poured into water, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:4-1:1, v/v) to give ethyl (2E)-3-[2-{[3-chloro-5-(trifluoromethyl)pyridin-2-yl]oxy}-4-(2-hydroxy-3-isopropoxypropoxy)phenyl]acrylate (3.16 g, yield: 81%) as a colorless oil.

¹H-NMR (300 MHz, CDCl₃) δ:1.17 (6H, d, J=6.3 Hz), 1.28 (3H, t, J=7.2 Hz), 2.54 (11H, d, J=4.5 Hz), 3.48-3.68 (3H, m), 4.00-4.24 (5H, m), 6.37 (1H, d, J=16.2 Hz), 6.72 (1H, s), 6.78-6.93 (1H, m), 7.611-7.72 (2H, m), 8.02 (1H, s), 8.25 (1H, s)

### Reference Example 319

To a mixture of ethyl (2E)-3-[2-{[3-chloro-5-(trifluoromethyl)pyridin-2-yl]oxy}-4-(3-cyanopropoxy)phenyl]acrylate (3.25 g), tetrahydrofuran (15 ml), and ethanol (10 ml) was added a 1M sodium hydroxide solution (14.3 ml), and the mixture was stirred at 50° C. for 2 hr. The reaction mixture was poured into water, and the mixture was neutralized with a 1M hydrochloric acid solution (14.4 ml) and extracted with ethyl acetate. The ethyl acetate layer was washed with saturated brine, dried (MgSO₄), and concentrated. The obtained solid was recrystallized from ethyl acetate-hexane to give (2E)-3-[2-{[3-chloro-5-(trifluoromethyl)pyridin-2-yl]oxy}-4-(3-cyanopropoxy)phenyl]acrylic acid (2.38 g, yield: 78%) as colorless crystals. melting point 183-185° C.

### Reference Example 320

To a mixture of ethyl (2E)-3-[2-{[3-chloro-5-(trifluoromethyl)pyridin-2-yl]oxy}-4-(2-oxopropoxy)phenyl]acrylate (2.46 g), tetrahydrofuran (40 ml), and ethanol (10 ml) was added a 1M sodium hydroxide solution (11.1 ml), and the mixture was stirred at 50° C. for 3 hr. The reaction mixture was poured into water, and the mixture was neutralized with a 1M hydrochloric acid solution (11.2 ml) and extracted with ethyl acetate. The ethyl acetate layer was washed with saturated brine, dried (MgSO₄), and concentrated. The obtained solid was recrystallized from ethyl acetate-hexane to give (2E)-3-[2-{[3-chloro-5-(trifluoromethyl)pyridin-2-yl]oxy}-4-(2-oxopropoxy)phenyl]acrylic acid (311 mg, yield: 14%) as colorless crystals. melting point 179-180° C.

### Reference Example 321

To a mixture of ethyl (2E)-3-{2-{[3-chloro-5-(trifluoromethyl)pyridin-2-yl]oxy}-4-[2-(2-methyl-1,3-dioxolan-2-yl)ethoxy]phenyl}acrylate (2.75 g), tetrahydrofuran (10 ml), and ethanol (10 ml) was added a 1M sodium hydroxide solution (11.0° ml), and the mixture was stirred at 50° C. for 3 hr. The reaction mixture was poured into water, and the mixture was neutralized with a 1M hydrochloric acid solution (11.1 ml) and extracted with ethyl acetate. The ethyl acetate layer was washed with saturated brine, dried (MgSO₄), and concentrated. The obtained solid was recrystallized from ethyl acetate-hexane to give (2E)-3-{2-{[3-chloro-5-(trifluoromethyl)pyridin-2-yl]oxy}-4-[2-(2-methyl-1,3-dioxolan-2-yl)ethoxy]phenyl}acrylic acid (1.79 g, yield: 69%) as colorless crystals. melting point 144-146° C.

### Reference Example 322

To a mixture of ethyl (2E)-3-[2-{[3-chloro-5-(trifluoromethyl)pyridin-2-yl]oxy}-4-(2-hydroxy-3-isopropoxypropoxy)phenyl]acrylate (3.15 g), tetrahydrofuran (10 ml), and ethanol (10 ml) was added a 1M sodium hydroxide solution (12.5 ml), and the mixture was stirred at 50° C. for 3 hr. The reaction mixture was poured into water, and the mixture was neutralized with a 1M hydrochloric acid solution (12.6 ml) and extracted with ethyl acetate. The ethyl acetate layer was washed with saturated brine, dried (MgSO₄), and concentrated. The obtained solid was recrystallized from ethyl acetate-hexane to give (2E)-3-[2-{[3-chloro-5-(trifluoromethyl)pyridin-2-yl]oxy}-4-(2-hydroxy-3-isopropoxypropoxy)phenyl]acrylic acid (2.18 g, yield: 73%) as colorless crystals. melting point 92-93° C.

### Reference Example 323

A mixture of (2E)-3-[2-{[3-chloro-5-(trifluoromethyl)pyridin-2-yl]oxy}74-(2-hydroxy-3-isopropoxypropoxy)phenyl]acrylic acid (772 mg), pyridine (0.014 ml), and acetic anhydride (4 ml) was stirred at room temperature for 3 hr. The reaction mixture was concentrated, and the residue was subjected to silica gel column chromatography, and eluted with ethyl acetate-hexane (1:4-1:1, v/v). The obtained oil was dissolved in tetrahydrofuran (20 ml), saturated aqueous sodium hydrogen carbonate (10 ml) was added, and the mixture was stirred at room temperature for 24 hr. The reaction mixture was concentrated, a 1M hydrochloric acid solution was added to the residue, and the mixture was extracted with ethyl acetate. The ethyl acetate layer was washed with saturated brine, dried (MgSO₄), and concentrated. The obtained solid was recrystallized from ethyl acetate-hexane to give (2E)-3-(4-[2-(acetyloxy)-3-isopropoxypropoxy]-2-{[3-chloro-5-(trifluoromethyl)pyridin-2-yl]oxy}phenyl)acrylic acid (441 mg, yield: 53%) as colorless crystals. melting point 134-135° C.

### Reference Example 324

To a mixture of 5-methyl-1H-pyrrole-2-carbaldehyde (2.00 g), 2,4-dichlorobenzyl chloride (3.95 g) and N,N-dimethylformamide (10 ml) was added sodium hydride (60% in oil, 806 mg) at 0° C., and the mixture was stirred at DOC for 1 hr and at room temperature for 8 hr. The reaction mixture was poured into water, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:10-1:4, v/v). The obtained solid was recrystallized from ethyl acetate-hexane to give 1-(2,4-dichlorobenzyl)-5-methyl-1H-pyrrole-2-carbaldehyde (4.35 g, yield: 89%) as pale-yellow crystals. melting point 86-87° C.

### Reference Example 325

A mixture of 1-(2,4-dichlorobenzyl)-5-methyl-1H-pyrrole-2-carbaldehyde (3.76 g), malonic acid (5.84 g), piperidine (3.46 ml) and pyridine (25 ml) was stirred at 110° C. for 8 hr. After cooling to room temperature, the reaction mixture was concentrated. A 1M hydrochloric acid solution was added to the residue, and the mixture was extracted with ethyl acetate. The ethyl acetate layer was washed with saturated brine, dried (MgSO₄), and concentrated. The obtained solid was recrystallized from ethyl acetate-hexane to give (2E)-3-[1-(2,4-dichlorobenzyl)-5-methyl-1H-pyrrol-2-yl]acrylic acid (2.51 g, yield: 58%) as yellow crystals. melting point 181-182° C.

### Reference Example 326

To a mixture of 3,5-dimethyl-1H-pyrrole-2-carbaldehyde (3.30 g), 2,4-dichlorobenzyl chloride (5.76 g), and N,N-dimethylformamide (30 ml) was added sodium hydride (60% in oil, 1.18 g) at 0° C., and the mixture was stirred at 0° C. for 1 hr and at room temperature for 15 hr. The reaction mixture was poured into water, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:10-1:5, v/v). The obtained solid was recrystallized from ethyl acetate-hexane to give 1-(2,4-dichlorobenzyl)-3,5-dimethyl-1H-pyrrole-2-carbaldehyde (5.16 g, yield: 65%) as yellow crystals. melting point 88-89° C.

### Reference Example 327

A mixture of 1-(2,4-dichlorobenzyl)-3,5-dimethyl-1H-pyrrole-2-carbaldehyde (5.71 g), malonic acid (8.41 g), piperidine (4.99 ml), and pyridine (30 ml) was stirred at 110° C. for 10 hr. After cooling to room temperature, and the reaction mixture was concentrated. A 1M hydrochloric acid solution was added to the residue, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:5-1:1, v/v). The obtained solid was recrystallized from ethyl acetate-hexane to give (2E)-3-[1-(2,4-dichlorobenzyl)-3,5-dimethyl-1H-pyrrol-2-yl]acrylic acid (914 mg, yield: 14%) as yellow crystals. melting point 193-194° C.

### Reference Example 328

To a solution of (2E)-3-{2-{[3-chloro-5-(trifluoromethyl)pyridin-2-yl]oxy}-4-[3-(methoxymethoxy)-1,1-dimethylpropoxy]phenyl}-N-(pentylsulfonyl)acrylamide (111 mg) in acetone (3 ml) was added 1N hydrochloric acid (2 ml), and the mixture was stirred at 50° C. for 6 hr. After allowing to cool to room temperature, a saturated aqueous sodium hydrogencarbonate solution was added to the reaction mixture, and the mixture was diluted with ethyl acetate. The organic layer was washed with saturated brine, dried (MgSO₄), filtrated and concentrated to give a white solid. Recrystallization from ethanol-water gave (2E)-3-(2-{[3-chloro-5-(trifluoromethyl)pyridin-2-yl]oxy}-4-hydroxyphenyl)-N-(pentylsulfonyl)acrylamide hemihydrate (69 mg, yield: 78%) as white crystals. melting point 80-83° C.

### Reference Example 329

A mixture of 2-{[3-chloro-5-(trifluoromethyl)pyridin-2-yl]oxy}-4-(2-methoxyethoxy)benzaldehyde (1.00 g), malonic acid (280 mg), pyrrolidine (0.225 ml) and acetic acid (5 ml) was stirred at 100° C. for 4.5 hr, and cooled to room temperature. 1N Hydrochloric acid (2 ml) and water (20 ml) were successively added to the reaction mixture, and the mixture was stirred at room temperature for 1 hr. The precipitated crystals were collected by filtration, washed twice with water (20 ml) and air-dried. Diisopropyl ether-hexane (1:1, v/v, 10 ml) was added to the obtained yellow powder, and the mixture was stirred for 1 hr. The crystals were collected by filtration, washed three times with diisopropyl ether-hexane (1:1, v/v, 10 ml) and dried to give (2E)-3-[2-{[3-chloro-5-(trifluoromethyl)pyridin-2-yl]oxy}-4-(2-methoxyethoxy)phenyl]acrylic acid (989 mg, yield: 88%) as pale-yellow crystals.

### Reference Example 330

To a solution of ethyl (2E)-3-[2-hydroxy-4-(2-methoxyethoxy)phenyl]acrylate (2.50 g) in N,N-dimethylformamide (20 ml) were added 2,5-dibromo-3-methyl-6-pyridine (3.06 g) and potassium carbonate (3.24 g), and the mixture was stirred at 100° C. for 72 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:3, v/v) to give ethyl (2E)-3-[2-[(5-bromo-3-methylpyridin-2-yl)oxy]-4-(2-methoxyethoxy)phenyl]acrylate (2.16 g, yield: 52%) as colorless crystals.

¹H-NMR (300 MHz, CDCl₃) δ:1.28 (3H, t, J=7.1 Hz), 2.38 (3H, s), 3.42 (3H, s), 3.71-3.74 (2H, m), 4.09-4.12 (2H, m), 4.19 (2H, q, J=7.1 Hz), 6.33 (1H, d, J=16.0 Hz), 6.59 (1 H, d, J=2.6 Hz), 6.81 (1H, dd, J=8.8, 2.6 Hz), 7.60 (1H, d, J=8.8 Hz), 7.66-7.67 (1H, m), 7.76 (1H, d, J=16.0 Hz), 7.99 (1H, d, J=1.9 Hz).

### Reference Example 331

To a mixed solution of ethyl (2E)-3-[2-[(5-bromo-3-methylpyridin-2-yl) oxy]-4-(2-methoxyethoxy)phenyl]acrylate (880 mg) in tetrahydrofuran (10 ml) and ethanol (10 ml) was added a 1N aqueous sodium hydroxide solution (4.0 ml), and the mixture was stirred at 60° C. for 1 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethyl acetate-hexane to give (2E)-3-[2-[(5-bromo-3-methylpyridin-2-yl)oxy]-4-(2-methoxyethoxy)phenyl]acrylic acid (785 mg, yield: 95%) as colorless crystals. melting point 157.0-157.9° C.

### Reference Example 332

To a solution of 2-[(3,5-dichloropyridin-2-yl)oxy]-4-(3-methoxypropoxy)benzaldehyde (1.50 g) in tetrahydrofuran (15 ml) were added methyl methoxyacetate (0.97 g) and sodium tert-butoxide (1.05 g), and the mixture was stirred at room temperature for 24 hr. Water was added to the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:1, v/v) to give methyl (2Z)-3-[2-hydroxy-4-(3-methoxypropoxy)phenyl]-2-methoxyacrylate (870 mg, yield: 69%) as colorless crystals.

¹H-NMR (300 MHz, CDCl₃) δ:2.00-2.05 (2H, m), 3.35 (3H, s), 3.54 (2H, t, J=6.1 Hz), 3.78 (3H, s), 3.86 (3H, s), 4.06 (2H, t, J=6.1 Hz), 6.45-6.48 (2H, m), 7.09 (1H, d, J=9.6 Hz), 7.14 (1H, s), 9.08 (1H, s).

### Reference Example 333

To a solution of methyl (2Z)-3-[2-hydroxy-4-(3-methoxypropoxy)phenyl]-2-methoxyacrylate (836 mg) in N,N-dimethylformamide (8 ml) were added 2,3-dichloro-5-(trifluoromethyl)pyridine (920 mg) and potassium carbonate (780 mg), and the mixture was stirred at 80° C. for 5 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:4, v/v) to give methyl (2Z)-3-[2-{[3-chloro-5-(trifluoromethyl)pyridin-2-yl]oxy}-4-(3-methoxypropoxy)phenyl]-2-methoxyacrylate (1.25 g, yield: 93%) as colorless crystals.

¹H-NMR (300 MHz, CDCl₃) δ:2.00-2.05 (2H, m), 3.34 (3H, s), 3.53 (2H, t, J=6.0 Hz), 3.73 (3H, s), 3.77 (3H, s), 4.07 (2H, t, J=6.0 Hz), 6.68 (1H, s), 6.88 (1H, dd, J=8.9, 2.6 Hz), 7.04 (1H, s), 8.00 (1H, d, J=2.6 Hz), 8.22 (1H, d, J=8.9 Hz), 8.25-8.26 (1H, m).

### Reference Example 334

To a mixed solution of methyl (2Z)-3-[2-{[3-chloro-5-(trifluoromethyl)pyridin-2-yl]oxy}-4-(3-methoxypropoxy)phenyl]-2-methoxyacrylate (1.07 g) in tetrahydrofuran (4 ml) and methanol (4 ml) was added a 1N aqueous sodium hydroxide solution (4.5 ml), and the mixture was stirred at 60° C. for 1 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:1, v/v). The obtained crude crystals were recrystallized from ethyl acetate-hexane to give (2Z)-3-[2-{[3-chloro-5-(trifluoromethyl)pyridin-2-yl]oxy}-4-(3-methoxypropoxy)phenyl]-2-methoxyacrylic acid (582 mg, yield: 56%) as colorless crystals. melting point 153.0-153.5° C.

### Reference Example 335

To a solution of ethyl 3-[2-(benzyloxy)-4-(2-methoxyethoxy)phenyl]propanoate (4.84 g) in toluene (50 ml) was added a 1.5 M diisobutylaluminum hydride solution in toluene (16.0 ml) at 0° C., and the mixture was stirred at 0° C. for 5 hr. Methanol and Celite were added to the reaction mixture, and the mixture was stirred for 30 min. Celite was filtered off, and the filtrate was concentrated. The obtained residue was subjected to silica gel column chromatography, and eluted with ethyl acetate-hexane (2:3, v/v) to give 3-[2-(benzyloxy)-4-(2-methoxyethoxy)phenyl]propan-1-ol (3.35 g, yield: 78%) as a colorless amorphous solid.

¹H-NMR (300 MHz, CDCl₃) δ:1.54 (1H, s), 1.78-1.86 (2H, m), 2.69 (2H, t, J=7.2 Hz), 3.44 (3H, s), 3.54-3.60 (2H, m), 3.71-3.74 (2H, m), 4.07-4.10 (2H, m), 5.02 (2H, s), 6.46 (1H, dd, J=8.3, 2.3 Hz), 6.58 (1H, d, J=2.3 Hz), 7.05 (1H, d, J=8.3 Hz), 7.30-7.50 (5H, m).

### Reference Example 336

To a solution of 3-[2-(benzyloxy)-4-(2-methoxyethoxy)phenyl]propan-1-ol (1.01 g) in tetrahydrofuran (15 ml) was added N, N′-carbonyldiimidazole (780 mg), and the mixture was stirred at room temperature for 2 hr. (Aminomethyl)cyclopropane (475 mg) was added to the reaction mixture, and the mixture was stirred at room temperature for 5 hr. The reaction mixture was concentrated, the obtained residue was subjected to silica gel column chromatography, and eluted with ethyl acetate-hexane (3:7, v/v) to give 3-[2-(benzyloxy)-4-(2-methoxyethoxy)phenyl]propyl (cyclopropylmethyl)carbamate as a colorless amorphous solid (1.20 g). The obtained amorphous solid was dissolved in methanol (100 ml), 10% palladium-carbon (130 mg) was added, and the mixture was stirred under a hydrogen atmosphere at room temperature for 1 hr. The catalyst was filtered off, and the filtrate was concentrated. The obtained residue was subjected to silica gel column chromatography, and eluted with ethyl acetate-hexane (1:2, v/v) to give 3-[2-hydroxy-4-(2-methoxyethoxy)phenyl]propyl (cyclopropylmethyl)carbamate (940 mg, yield: 99%) as colorless crystals.

¹H-NMR (300 MHz, CDCl₃) δ:0.16-0.22 (2H, m), 0.48-0.53 (2H, m), 0.91-0.99 (1H, m), 1.86-1.95 (2H, m), 2.61 (2H, t, J=7.3 Hz), 3.02-3.07 (2H, m), 3.44 (3H, s), 3.71-3.74 (2H, m), 4.05-4.11 (4H, m), 4.76-4.83 (1H, m), 5.59 (1H, s), 6.41-6.46 (2H, m), 6.97, (1H, d, J=8.1 Hz).

### Reference Example 337

Under ice-cooling, to a mixture of 2-{[3-chloro-5-(trifluoromethyl)pyridin-2-yl]oxy}-4-(methoxymethoxy)benzaldehyde (10.0 g), triethyl 2-phosphonopropionate (7.24 g), tetrahydrofuran (30 ml) and N,N-dimethylformamide (30 ml) was added lithium hydroxide monohydrate (1.28 g), and the mixture was stirred for 2 hr. 0.1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-3:7, v/v), concentrated, and crystallized from ethyl acetate-hexane to give a mixture (11.4 g) of ethyl (2E)-3-[2-{[3-chloro-5-(trifluoromethyl)pyridin-2-yl]oxy}-4-(methoxymethoxy)phenyl]-2-methylacrylate and ethyl (2Z)-3-[2-{[3-chloro-5-(trifluoromethyl)pyridin-2-yl]oxy}-4-(methoxymethoxy)phenyl]-2-methylacrylate.

To a solution of the obtained mixture (5.00 g) in ethanol (50 ml) was added concentrated hydrochloric acid (2.5 ml), and the mixture was stirred at 50° C. for 5 hr. The reaction mixture was concentrated, and the residue was subjected to silica gel column chromatography, and eluted with ethyl acetate-hexane (0:1-3:7, v/v), concentrated, and crystallized from ethyl acetate-hexane to give ethyl (2E)-3-(2-{[3-chloro-5-(trifluoromethyl)pyridin-2-yl]oxy}-4-hydroxyphenyl)-2-methylacrylate (3.04 g, 73%) as colorless crystals.

¹H-NMR (300 MHz, CDCl₃) δ:1.26 (3H, t, J=7.2 Hz), 2.01 (3H, d, J=1.5 Hz), 4.17 (2H, q, J=7.0 Hz), 5.11 (1H, s), 6.72 (1H, d, J=2.4 Hz), 6.80 (1H, dd, J=8.5, 2.4 Hz), 7.35 (1H, d, J=8.5 Hz), 7.50 (1H, d, J=1.3 Hz), 7.97 (1H, d, J=2.3 Hz), 8.24 (1H, dd, J=2.1, 1.1 Hz).

### Reference Example 338

To a solution of ethyl (2E)-3-(2-{[3-chloro-5-(trifluoromethyl)pyridin-2-yl]oxy}-4-hydroxyphenyl)-2-methylacrylate (3.00 g) in N,N-dimethylformamide (50 ml) were added isopropyliodide (1.50 ml) and potassium carbonate (4.12 g), and the mixture was stirred at 50° C. for 2 hr. The reaction mixture was filtrated, ethyl acetate was added to the filtrate. The mixture was washed successively with saturated brine and water, dried (MgSO₄), and concentrated. The residue was subjected to silica gel column chromatography, and eluted with ethyl acetate-hexane (0:1-2:8, v/v), concentrated, and crystallized from ethyl acetate-hexane to give ethyl (2E)-3-(2-{[3-chloro-5-(trifluoromethyl)pyridin-2-yl]oxy}-4-isopropoxyphenyl)-2-methylacrylate (3.17 g, 96%) as colorless crystals.

¹H-NMR (300 MHz, CDCl₃) δ:1.25 (3H, t, J=7.1 Hz), 1.36 (6H, d, J=6.0 Hz); 1.96-2.09 (3H, m), 4.02-4.27 (2H, m), 4.42-4.67 (1H, m), 6.72 (1H, d, J=2.4 Hz), 6.84 (1H, dd, J=8.6, 2.4 Hz), 7.39 (1H, d, J=8.7 Hz), 7.51 (1H, s), 7.96 (1H, d, J=2.1 Hz), 8.23 (1H, d, J=0.9 Hz).

### Reference Example 339

To a mixture of ethyl (2E)-3-(2-{[3-chloro-5-(trifluoromethyl)pyridin-2-yl]oxy}-4-isopropoxyphenyl)-2-methylacrylate (3.00 g), ethanol (20 ml), tetrahydrofuran (10 ml) and water (10 ml) was added lithium hydroxide monohydrate (2.27 g), and the mixture was stirred at room temperature for 4 hr. The reaction mixture was acidified with 1N hydrochloric acid, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-2:8, v/v), concentrated, and crystallized from ethyl acetate-hexane to give (2E)-3-(2-{[3-chloro-5-(trifluoromethyl)pyridin-2-yl]oxy}-4-isopropoxyphenyl)-2-methylacrylic acid (2.00 g, 71%) as colorless crystals.

¹H-NMR (300 MHz, CDCl₃) δ:1.36 (6H, d, J=6.0 Hz), 2.05 (3H, d, J=1.5 Hz), 4.48-4.64 (1H, m), 6.70 (1H, d, J=2.4 Hz), 6.85 (1H, dd, J=8.7, 2.4 Hz), 7.41 (1H, d, J=8.7 Hz), 7.64 (1H, s), 7.97 (1H, d, J=1.9 Hz), 8.19-8.28 (1H, m).

### Reference Example 340

To a solution of benzyl alcohol (2.00 g) in acetonitrile (100 ml) was added chlorosulfonylisocyanate (2.75 g) under ice-cooling, and the mixture was stirred for 30 min. Pyridine (5.3 ml) was added to the reaction mixture, and the mixture was stirred for 1 hr. 2-(Aminoethyl)-5-methylpyrazine (11.4 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (2:8-1:0, v/v), concentrated, and crystallized from ethyl acetate-hexane to give benzyl ({[(5-methylpyrazin-2-yl)methyl]amino}sulfonyl)carbamate (3.27 g, 53%) as colorless crystals.

¹H-NMR (300 MHz, CDCl₃) δ:2.55 (3H, s), 4.38-4.49 (2H, m), 5.15 (2H, s), 6.06 (1H, br.s.), 7.32-7.47 (6H, m), 8.30 (1H, s), 8.46 (1H, s).

### Reference Example 341

To a solution of benzyl alcohol (2.00 g) in acetonitrile (100 ml) was added chlorosulfonylisocyanate (2.75 g) under ice-cooling, and the mixture was stirred for 30 min. Pyridine (5.3 ml) was added to the reaction mixture, the mixture was stirred for 1 hr, 2-(2-aminoethyl)pyridine (11.3 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with methanol-ethyl acetate (0:1-1:0, v/v) and concentrated to give benzyl {[(2-pyridin-2-ylethyl)amino]sulfonyl}carbamate (4.58 g, 74%) as a yellow oil.

¹H-NMR (300 MHz, CDCl₃) δ:3.03 (2H, t, J=6.3 Hz), 3.44-3.55 (2H, m), 5.12 (2H, s), 7.08-7.18 (2H, m), 7.32 (5H, s), 7.55-7.66 (1H, s), 8.40-8.48 (1H, m).

### Reference Example 342

To a solution of benzyl alcohol (2.00 g) in acetonitrile (100 ml) was added chlorosulfonylisocyanate (2.75 g) under ice-cooling, and the mixture was stirred for 30 min. Pyridine (5.3 ml) was added to the reaction mixture, the mixture was stirred for 1 hr, 2-(2-aminoethyl)pyridine (11.3 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-1:1, v/v), concentrated, and crystallized from ethyl acetate-hexane to give benzyl (pyrrolidin-1-ylsulfonyl)carbamate (2.99 g, 54%) as colorless crystals.

¹H-NMR (300 MHz, CDCl₃) δ:1.80-1.96 (4H, m), 3.41-3.54 (4H, m), 5.17 (2H, s), 7.16 (1H, br.s.), 7.32-7.43 (5H, s).

### Reference Example 343

A suspension of benzyl ({[(5-methylpyrazin-2-yl)methyl]amino}sulfonyl)carbamate (3.00 g) and 10% palladium-carbon (0.30 g) in methanol (80 ml) was stirred under a hydrogen atmosphere at room temperature, for 6 hr. The reaction mixture was filtrated, and the solvent was evaporated under reduced pressure. The residue was subjected to silica gel column chromatography, eluted with methanol-ethyl acetate (0:1-2:8, v/v), concentrated and crystallized from ethyl acetate-hexane to give N-[(5-methylpyrazin-2-yl)methyl]sulfamide (1.29 g, 72%) as colorless crystals.

¹H-NMR (300 MHz, CDCl₃) δ:2.59 (3H, s), 4.47 (2H, s), 4.68 (2H, br.s.), 5.33 (1H, br.s.), 8.41 (1H, s), 8.50 (1H, s)

### Reference Example 344

A suspension of benzyl {[(2-pyridin-2-ylethyl)amino]sulfonyl}carbamate (4.58 g) and 10%, palladium-carbon (0.46 g) in ethanol (100 ml) was stirred under a hydrogen atmosphere at room temperature for 6 hr. The reaction mixture was filtrated, and the solvent was evaporated under reduced pressure. The residue was subjected to silica gel column chromatography, eluted with methanol-ethyl acetate (0:1-1:0, v/v) and concentrated to give N-(2-pyridin-2-ylethyl)sulfamide (1.46 g, 53%) as a colorless solid.

¹H-NMR (300 MHz, DMSO-d₆) δ:2.94 (2H, t, J=7.6 Hz), 3.16-3.29 (2H, m), 6.41-6.69 (3H, m), 7.13-7.34 (2H, m), 7.62-7.78 (1H, m), 8.49 (1H, d, J=4.5 Hz).

### Reference Example 345

A suspension of benzyl (pyrrolidin-1-ylsulfonyl)carbamate

(2.50 g) and 10,% palladium-carbon (0.30 g) in ethanol (80 ml) was stirred under a hydrogen atmosphere at room temperature for 6 hr. The reaction mixture was filtrated, and the solvent was evaporated under reduced pressure. The residue was crystallized from ethyl acetate-hexane to give pyrrolidine-1-sulfonamide (1.04 g, 79%) as colorless crystals.

¹H-NMR (300 MHz, CDCl₃) δ:1.81-2.02 (4H, m), 3.19-3.37 (4H, m), 4.50 (2H, br.s.).

### Reference Example 346

A mixture of ethyl (2E)-3-(2-{[3-chloro-5-(trifluoromethyl)pyridin-2-yl]oxy}-4-hydroxyphenyl)acrylate (11.4 g), platinum (IV) oxide (133 mg), and ethanol-tetrahydrofuran (292 ml, v/v=1/1) was stirred under a hydrogen atmosphere at room temperature for 6 hr. The catalyst was filtered off, and the filtrate was concentrated. The obtained residue was subjected to silica gel column chromatography, and eluted with ethyl acetate-hexane (1:3, v/v) to give ethyl 3-(2-{[3-chloro-5-(trifluoromethyl)pyridin-2-yl]oxy}-4-hydroxyphenyl)propanoate (10.8 g, yield: 94%) as a colorless oil.

¹H-NMR (300 MHz, CDCl₃) δ:1.14-1.31 (3H, m), 2.57 (2H, t, J=7.6 Hz), 2.75 (2H, t, J=7.7 Hz), 4.02-4.18 (2H, m), 5.48 (1H, d, J=7.0 Hz), 6.59 (1H, d, J=2.4 Hz), 6.68 (1H, dd, J=8.3, 2.4 Hz), 7.17 (1H, d, J=8.3 Hz), 7.99 (1H, s), 8.27 (1H, s).

### Reference Example 347

To a mixture of ethyl 3-(2-{[3-chloro-5-(trifluoromethyl)pyridin-2-yl]oxy}-4-hydroxyphenyl)propanoate (1.01 g), 1-butanol (0.29 g), tributylphosphine (1.05 g) and tetrahydrofuran (10 ml) was added 1,1′-(azodicarbonyl)dipiperidine (1.31 g) at room temperature, and the mixture was stirred for 30 min. The reaction mixture was concentrated, diisopropyl ether was added, and the precipitated crystals were filtered off. The filtrate was concentrated, and the obtained residue was subjected to silica gel column chromatography and eluted with ethyl acetate-hexane (1:9-3:7, v/v) to give ethyl 3-(4-butoxy-2-{[3-chloro-5-(trifluoromethyl)pyridin-2-yl]oxy}phenyl)propanoate (1.1 g, yield-95%) as a colorless oil.

¹H-NMR (300 MHz, CDCl₃) δ:0.96 (3H, t, J=7.4 Hz), 1.21 (3H, t, J=7.2 Hz), 1.37-1.81 (4H, m), 2.52-2.61 (2H, m), 2.70-2.80 (2H, m), 3.87-3.97 (2H, m), 4.03-4.15 (2H, m), 6.64 (1H, d, J=2.6 Hz), 6.78 (1H, dd, J=8.5, 2.6 Hz), 7.22 (1H, d, J=8.5 Hz), 7.98 (1H, d, J=2.1 Hz), 8.27 (1H, d, J=1.1 Hz).

### Reference Example 348

Ethyl 3-(4-butoxy-2-{[3-chloro-5-(trifluoromethyl)pyridin-2-yl]oxy}phenyl)propanoate (1.1 g) was dissolved in diethyl ether (5 ml), a 1.5 M solution (4.1 ml) of diisobutylaluminum hydride in toluene was added at 0° C., and the mixture was stirred at room temperature for 1.5 hr. Methanol and water were added to the reaction mixture, and the mixture was stirred for a while, filtered through celite, and concentrated. The obtained residue was subjected to silica gel column chromatography, and eluted with ethyl acetate-hexane (1:3-2:3, v/v) to give colorless crystals. Recrystallization from ethyl acetate-hexane gave 3-(4-butoxy-2-{[3-chloro-5-(trifluoromethyl)pyridin-2-yl]oxy}phenyl)propan-1-ol (0.69 g, yield: 69%) as colorless crystals. melting point 72.6-72.7° C.

### Reference Example 349

To a solution of ethyl 3-(2-{[3-chloro-5-(trifluoromethyl)pyridin-2-yl]oxy}-A-hydroxyphenyl)propanoate (1.03 g) in N,N-dimethylformamide (5.3 ml) were added 1-bromo-3-methoxypropane (0.60 g), potassium carbonate (0.73 g) and sodium iodide (0.80 g), and the mixture was stirred at 80° C. for 18 hr. Water was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (3:17-2:3, v/v) to give ethyl 3-[2-{[3-chloro-5-(trifluoromethyl)pyridin-2-yl]oxy}-4-(3-methoxypropoxy)phenyl]propanoate (0.84 g, yield: 69%) as a colorless oil.

¹H-NMR (300 MHz, CDCl₃) δ:1.21 (3H, t, J=7.0 Hz), 1.98-2.08 (2H, m), 2.56 (2H, t, J=7.8 Hz), 2.76 (2H, t, J=7.6 Hz), 3.34 (3H, s), 3.53 (2H, t, J=6.1 Hz), 3.96-4.14 (4H, m), 6.65 (1H, d, J=2.7 Hz), 6.79 (1H, dd, J=8.5, 2.5 Hz), 7.22 (1H, d, J=8.3 Hz), 7.98 (1H, d, J=2.3 Hz), 8.27 (1H, s).

### Reference Example 350

Ethyl 3-[2-{[3-chloro-5-(trifluoromethyl)pyridin-2-yl]oxy}-4-(3-methoxypropoxy)phenyl]propanoate (840 mg) was dissolved in diethyl ether (3.6 ml), a 1.5 M diisobutylaluminum hydride toluene solution (3.0 ml) was added at 0° C., and the mixture was stirred at room temperature for 2.5 hr. Methanol and water were added to the reaction mixture, and the mixture was stirred for awhile, filtered through celite, and concentrated. The obtained residue was subjected to silica gel column chromatography, and eluted with ethyl acetate-hexane (1:3-1:1, v/v) to give colorless crystals. Recrystallization from ethyl acetate-hexane gave 3-[2-{[3-chloro-5-(trifluoromethyl)pyridin-2-yl]oxy}-4-(3-methoxypropoxy)phenyl]propan-1-ol (665 mg, yield: 87%) as colorless crystals. melting point 68.7-70.9° C.

### Reference Example 351

To a solution of ethyl 3-(2-{[3-chloro-5-(trifluoromethyl)pyridin-2-yl]oxy}-4-hydroxyphenyl)propanoate (1.03 g) in N,N-dimethylformamide (5.3 ml) was added sodium hydride (60% in oil, 0.15 g), and the mixture was stirred at 0° C. for 30 min. Further, 4-bromo-1-butene (0.53 g) was added, and the mixture was stirred at 50° C. for 48 hr. Water was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:9-3:7, v/v) to give ethyl 3-(4-(but-3-en-1-yloxy)-2-{[3-chloro-5-(trifluoromethyl)pyridin-2-yl]oxy}phenyl)propanoate (0.68 g, yield: 58%) as a colorless oil.

¹H-NMR (300 MHz, CDCl₃) δ:1.21 (3H, t, J=7.1 Hz), 2.47-2.61 (4H, m), 2.76 (2H, t, J=7.6 Hz), 3.95-4.01 (2H, m), 4.04-4.13 (2H, m), 5.06-5.20 (2H, m), 5.80-5.96 (1H, m), 6.65 (1H, d, J=2.6 Hz), 6.79 (1H, dd, J=8.5, 2.6 Hz), 7.22 (1H, d, J=8.5 Hz), 7.98 (1H, d, J=2.1 Hz), 8.27 (1 H, d, J=1.1 Hz).

### Reference Example 352

Ethyl 3-(4-(but-3-en-1-yloxy)-2-{[3-chloro-5-(trifluoromethyl)pyridin-2-yl]oxy}phenyl)propanoate (683 mg) was dissolved in diethyl ether (3.0 ml), a 1.5 M solution (2.6 ml) of diisobutylaluminum hydride in toluene was added at 0° C., and the mixture was stirred at room temperature for 4 hr. Methanol and water were added to the reaction mixture, and the mixture was stirred for a while, filtered through celite, and concentrated. The obtained residue was subjected to silica gel column chromatography, and eluted with ethyl acetate-hexane (1:3-2:3, v/v) to give colorless crystals. Recrystallization from ethyl acetate-hexane gave 3-(4-(but-3-en-1-yloxy)-2-{[3-chloro-5-(trifluoromethyl)pyridin-2-yl]oxy}phenyl)propan-1-ol (525 mg, yield: 85%) as colorless crystals. melting point 75.2-75.8° C.

### Reference Example 353

To a mixture of ethyl 3-(2-{[3-chloro-5-(trifluoromethyl)pyridin-2-yl]oxy}-4-hydroxyphenyl) propanoate (1.09 g), 2-furylmethanol (0.41 g), tributylphosphine (1.14 g) and tetrahydrofuran (11 ml) was added 1,1′-(azodicarbonyl)dipiperidine (1.42 g) at room temperature, and the mixture was stirred for 30 min. The reaction mixture was concentrated, diisopropyl ether was added, and the precipitated crystals were filtered off. The filtrate was concentrated, and the obtained residue was subjected to silica gel column chromatography and eluted with ethyl acetate-hexane (1:9-1:4, v/v) to give ethyl 3-[2-{[3-chloro-5-(trifluoromethyl)pyridin-2-yl]oxy}-4-(2-furylmethoxy)phenyl]propanoate (1.05 g, yield: 80%) as a colorless oil.

¹H-NMR (300 MHz, CDCl₃) δ:1.17-1.24 (3H, m), 2.53-2.62 (2H, m), 2.78 (2H, t, J=7.6 Hz), 4.04-4.15 (2H, m), 4.96 (2H, s), 6.35-6.43 (2H, m), 6.74 (1H, d, J=2.6 Hz), 6.87 (1H, dd, J=8.5, 2.6 Hz), 7.21-7.27 (1H, m), 7.41-7.46 (1H, m), 7.99 (1H, d, J=2.3 Hz), 8.26 (1H, dd, J 2.3, 0.9 Hz)

### Reference Example 354

Ethyl 3-[2-{[3-chloro-5-(trifluoromethyl)pyridin-2-yl]oxy}-4-(2-furylmethoxy)phenyl]propanoate (1050 mg) was dissolved in diethyl ether (4.5 ml), a 1.5 M solution (3.7 ml) of diisobutylaluminum hydride in toluene was added at 0° C., and the mixture was stirred at room temperature for 2.5 hr. Methanol and water were added to the reaction mixture, and the mixture was stirred for a while, filtered through celite, and concentrated. The obtained residue was subjected to silica gel column chromatography, and eluted with ethyl acetate-hexane (1:3-2:3, v/v) to give colorless crystals. Recrystallization from ethyl acetate-hexane gave 3-[2-{[3-chloro-5-(trifluoromethyl)pyridin-2-yl]oxy}-4-(2-furylmethoxy)phenyl]propan-1-ol (734 mg, yield: 77%) as colorless crystals. melting point 68.0-68.6° C.

### Reference Example 355

To a mixture of ethyl 3-(2-{[3-chloro-5-(trifluoromethyl)pyridin-2-yl]oxy}-4-hydroxyphenyl)propanoate (1.09 g), 4-(2-hydroxyethyl)morpholine (0.55 g), tributylphosphine (1.13 g) and tetrahydrofuran (11 ml) was added 1,1′-(azodicarbonyl)dipiperidine (1.41 g) at room temperature, and the mixture was stirred for 2 hr. The reaction mixture was concentrated, diisopropyl ether was added, and the precipitated crystals were filtered off. The filtrate was concentrated, and the obtained residue was subjected to silica gel column chromatography and eluted with ethyl acetate-hexane (1:1-7:3, v/v) to give ethyl 3-[2-{[3-chloro-5-(trifluoromethyl)pyridin-2-yl]oxy}-4-(2-morpholin-4-ylethoxy)phenyl]propanoate (1.14 g, yield: 81%) as a colorless oil.

¹H-NMR (300 MHz, CDCl₃) δ:1.17-1.30 (3H, m), 2.52-2.61 (6H, m), 2.72-2.82 (4H, m), 3.69-3.75 (4H, m), 4.04-4.16 (4H, m), 6.67 (1H, d, J=2.6 Hz), 6.79 (1H, dd, J=8.5, 2.6 Hz), 7.23 (1H, d, J=8.5 Hz), 7.99 (1H, d, J=2.3 Hz), 8.26 (1H, dd, J=2.2, 1.0 Hz)

### Reference Example 356

Ethyl 3-[2-{[3-chloro-5-(trifluoromethyl)pyridin-2-yl]oxy}-4-(2-morpholin-4-ylethoxy)phenyl]propanoate (1139 mg) was dissolved in diethyl ether (4.5 ml), a 1.5 M solution (3.8 ml) of diisobutylaluminum hydride in toluene was added at 0° C., and the mixture was stirred at room temperature for 2.5 hr. Methanol and water were added to the reaction mixture, and the mixture was stirred for a while, filtered through celite, and concentrated. The obtained residue was subjected to silica gel column chromatography, and eluted with ethyl acetate to give colorless crystals. Recrystallization from ethyl acetate-hexane gave 3-[2-{[3-chloro-5-(trifluoromethyl)pyridin-2-yl]oxy}-4-(2-morpholin-4-ylethoxy)phenyl]propan-1-ol (737 mg, yield: 71%) as colorless crystals. melting point 69.2-70.4° C.

### Reference Example 357

To a mixture of ethyl 3-(2-{[3-chloro-5-(trifluoromethyl)pyridin-2-yl]oxy}-4-hydroxyphenyl)propanoate (1.05 g), tetrahydrofurfuryl alcohol (0.41 g), tributylphosphine (1.09 g) and tetrahydrofuran (10 ml) was added 1,1′-(azodicarbonyl)dipiperidine (1.36 g) at room temperature, and the mixture was stirred for 20 hr. The reaction mixture was concentrated, diisopropyl ether was added, and the precipitated crystals were filtered off. The filtrate was concentrated, and the obtained residue was subjected to silica gel column chromatography and eluted with ethyl acetate-hexane (3:17-1:4, v/v) to give ethyl 3-[2-{[3-chloro-5-(trifluoromethyl)pyridin-2-yl]oxy}-4-(tetrahydrofuran-2-ylmethoxy)phenyl]propanoate (789 mg, yield: 62%) as a colorless oil.

¹H-NMR (300 MHz, CDCl₃) δ:1.21 (3H, t, J=7.2 Hz), 1.86-2.17 (4H, m), 2.52-2.61 (2H, m), 2.76 (2H, t, J=7.7 Hz), 3.75-3.96 (4H, m), 4.04-4.14 (2H, m), 4.22-4.30 (1H, m), 6.68 (1H, d, J=2.6 Hz), 6.81 (1H, dd, J=8.5, 2.6 Hz), 7.22 (1H, d, J=8.7 Hz), 7.98 (1H, d, T=1.9 Hz), 8.26 (1H, dd, J=2.1, 0.9 Hz).

### Reference Example 358

Ethyl 3-[2-{[3-chloro-5-(trifluoromethyl)pyridin-2-yl]oxy}-4-(tetrahydrofuran-2-ylmethoxy)phenyl]propanoate (739 mg) was dissolved in diethyl ether (3.0 ml), a 1.5 M solution (2.6 ml) of diisobutylaluminum hydride in toluene was added at 0° C., and the mixture was stirred at room temperature for 3 hr. Methanol and water were added to the reaction mixture, and the mixture was stirred for a while, filtered through celite, and concentrated. The obtained residue was subjected to silica gel column chromatography, and eluted with ethyl acetate-hexane (1:3-2:3, v/v) to give colorless crystals. Recrystallization from ethyl acetate-hexane gave 3-[2-{[3-chloro-5-(trifluoromethyl)pyridin-2-yl]oxy}-4-(tetrahydrofuran-2-ylmethoxy)phenyl]propan-1-ol (495 mg, yield: 73%) as colorless crystals. melting point 91.3-93.6° C.

### Reference Example 359

A suspension of ethyl (2E)-3-(2-{[3-chloro-5-(trifluoromethyl)pyridin-2-yl]oxy}-4-{2-[(triisopropylsilyl)oxy]ethoxy}phenyl)acrylate (12.1 g) and 10% palladium-carbon (1.21 g) in ethanol (250 ml) was stirred under a hydrogen atmosphere at room temperature for 6 hr. The reaction mixture was filtrated, and the solvent was evaporated under reduced pressure to give ethyl 3-(2-{[3-chloro-5-(trifluoromethyl)pyridin-2-yl]oxy}-4-{2-[(triisopropylsilyl)oxy]ethoxy}phenyl)propanoate (12.1 g, quant.) as a colorless oil.

¹H-NMR (300 MHz, CDCl₃) δ:1.00-1.10 (21H, m), 1.21 (3H, t, J=7.2 Hz), 2.47-2.64 (2H, m), 2.76 (2H, t, J=7.8 Hz), 3.86-4.21 (6H, m), 6.67 (1H, d, J=2.6 Hz), 6.76-6.87 (1H, m), 7.22 (1H, d, J=8.7 Hz), 7.98 (1H, d, J=2.1 Hz), 8.26 (1H, d, J=1.1 Hz).

### Reference Example 360

To a solution of ethyl 3-(2-{[3-chloro-5-(trifluoromethyl)pyridin-2-yl]oxy}-4-{2-[(triisopropylsilyl)oxy]ethoxy}phenyl)propanoate (12.1 g) in diethyl ether (50 ml) was added dropwise a 1.5 M solution (34.3 ml) of diisobutylaluminum hydride in toluene, and the mixture was stirred while warming to room temperature over 4 hr. Saturated brine was added to the reaction mixture, the mixture was filtrated, and the filtrate was extracted with ethyl acetate. The ethyl acetate layer was washed with saturated brine, dried (MgSO₄), and concentrated. The residue was subjected to silica gel column chromatography, eluted with ethyl acetate-hexane (0:1-2:8, v/v) and concentrated to give 3-(2-{[3-chloro-5-(trifluoromethyl)pyridin-2-yl]oxy}-4-{2-[(triisopropylsilyl)oxy]ethoxy}phenyl)propan-1-ol (8.74 g, 77%) as a pale-yellow oil.

¹H-NMR (300 MHz, CDCl₃) δ:0.98-1.9 (21H, m), 1.37 (1H, t, J=5.7 Hz), 1.73-1.92 (2H, m), 2.49-2.57 (2H, m), 3.60 (2H, q, J=6.2 Hz), 4.01-4.04 (4H, m), 6.66 (1H, d, J=2.4 Hz), 6.77-6.88 (1H, m), 7.22 (1H, d, J=8.7 Hz), 7.98 (1H, d, J=1.9 Hz), 8.26 (1. H, dd, J=2.2, 1.0 Hz).

### Example 1

To a solution of 3-[2-{[3-chloro-5-(trifluoromethyl)pyridin-2-yl]oxy}-4-(2-methoxyethoxy)phenyl]propionic acid (3.49 g) in tetrahydrofuran (40 ml) was added N,N′-carbonyldiimidazole (2.08 g), and the mixture was heated under reflux for 1 hr. After cooling to room temperature, pentane-1-sulfonamide (1.63 g), and 1,8-diazabicyclo[5.4.0]undec-7-ene (2.0 ml) were added to the reaction mixture, and the mixture was stirred overnight. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:4-1:1, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]-N-(pentylsulfonyl)propanamide (1.62 g, yield: 35%) as white feather crystals. melting point 140-142° C.

### Example 2

To a solution of 3-(2-{[3-chloro-5-(trifluoromethyl)pyridin-2-yl]oxy}-4-isopropoxyphenyl)propionic acid (530 mg) in tetrahydrofuran (10 ml) was added N,N′-carbonyldiimidazole (313 mg), and the mixture was heated under reflux for 1 hr. After cooling to room temperature, pentane-1-sulfonamide (225 mg) and 1,8-diazabicyclo[5.4.0]undec-7-ene (0.30 ml) were added to the reaction mixture, and the mixture was stirred overnight. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/V) to give a white solid. Recrystallization from ethyl acetate-hexane gave 3-(2-{[3-chloro-5-(trifluoromethyl)pyridin-2-yl]oxy}-4-isopropoxyphenyl)-N-(pentylsulfonyl)propanamide (353 mg, yield: 50%) as white feather crystals. melting point 94-95° C.

¹H-NMR (300 MHz, CDCl₃) δ:0.85-0.94 (3H, m), 1.24-1.41 (10H, m), 1.60-1.73 (2H, m), 2.55 (2H, t, J=7.2 Hz), 2.96 (2H, t, J=7.1 Hz), 3.12-3.20 (2H, m), 4.36-4.55 (1H, m), 6.45 (1H, d, J=2.4 Hz), 6.74 (1H, dd, J=8.4, 2.5 Hz), 7.17 (1H, d, J=8.5 Hz), 8.08 (1H, d, J=2.1 Hz), 8.33-8.43 (1H, m).

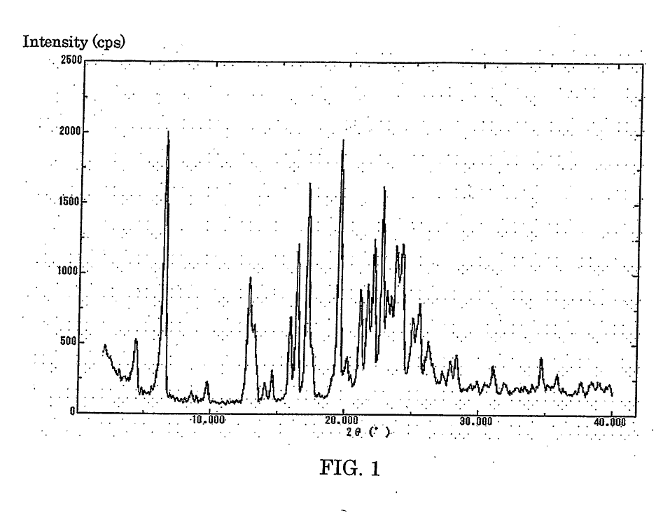

Recrystallization of the crude crystals obtained under the same conditions as in Example 2 from ethyl acetate-hexane gave crystals. The X-ray powder diffraction pattern of this crystal as measured using Cu-Kα ray (tube voltage: 40 KV; tube current: 50 mA) as a radiation source and RINT2100 type Ultima+ (Rigaku Corporation) is shown in FIG. 1.

Data (main peak) of X-ray powder diffraction

| diffraction angle: 2θ (°) | spacing: d value (angstrom)) |
|---|---|
| 6.50 | 13.6 |
| 12.8 | 6.90 |
| 16.4 | 5.39 |
| 17.2 | 5.15 |
| 19.5 | 4.54 |
| 22.1 | 4.01 |
| 22.7 | 3.91 |
| 24.2 | 3.67 |

### Example 3

To a solution of 3-[2-{[3-chloro-5-(trifluoromethyl)pyridin-2-yl]oxy}-4-(2-methoxyethoxy)phenyl]propionic acid (502 mg) in tetrahydrofuran (10 ml) was added N,N′-carbonyldiimidazole (213 mg), and the mixture was heated under reflux for 1 hr. After cooling to room temperature, benzenesulfonamide (212 mg) and 1,8-diazabicyclo[5.4.0]undec-7-ene (0.25 ml) were added to the reaction mixture, and the mixture was stirred overnight. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (hexane alone to 1:4, v/v) to give a colorless oil. This was subjected to basic silica gel column chromatography, and eluted with ethyl acetate-methanol (ethyl acetate alone to 4:1, v/v) and then with 2.0 M ammonia methanol solution to give a yellow oil. The obtained oil was subjected to silica gel column chromatography, and eluted with ethyl acetate-hexane (1:9-1:1, v/v) to give 3-[2-{[3-chloro-5-(trifluoromethyl)pyridin-2-yl]oxy}-4-(2-methoxyethoxy)phenyl]-N-(phenylsulfonyl)propanamide (338 mg, yield: 51%) as a colorless oil.

¹H-NMR (300 MHz, CDCl₃) δ:2.46 (2H, t, J=7.2 Hz), 2.84 (2H, t, J=7.1 Hz), 3.44 (3H, s), 3.73 (2H, dd, J=5.4, 3.9 Hz), 4.04 (2H, dd, J=5.4, 3.9 Hz), 6.33-6.46 (2H, m), 6.78 (1H, d, J=8.5 Hz), 7.44-7.55 (1H, m), 7.60-7.69 (1H, m), 7.82-7.89 (2H, m), 8.08 (1H, d, J=1.9 Hz), 8.42-8.51 (1H, m), 9.39 (1H, s).

### Example 4

To a solution of 3-(2-{[3-chloro-5-(trifluoromethyl)pyridin-2-yl]oxy}-4-isopropoxyphenyl)propionic acid (1.47 g) in tetrahydrofuran (20 ml) were, added thionyl chloride (0.75 ml) and N,N-dimethylformamide (0.10 ml) under ice-cooling, and the mixture was stirred for 2 hr. The reaction mixture was concentrated, and the residue was dissolved in tetrahydrofuran (20 ml). Benzenesulfonamide (685 mg), N,N-diisopropylethylamine (0.80 ml) and 4-dimethylaminopyridine (431 mg) were added to the reaction mixture at room temperature, and the mixture was stirred for 3 hr. A saturated aqueous ammonium chloride solution was added to the reaction mixture, and the mixture was diluted with ethyl acetate. The organic layer was washed with water and saturated brine, dried (MgSO₄), filtrated and concentrated. The obtained residue was subjected to silica gel column chromatography, and eluted with ethyl acetate-hexane (1:9-3:2, v/v) to give a pale-yellow oil. This was dissolved in a suspension of activated carbon in ethyl acetate, the mixture was stirred at room temperature for 30 min, filtrated and concentrated to give a pale-yellow solid. Recrystallization from ethyl acetate-hexane gave 3-(2-{[3-chloro-5-(trifluoromethyl)pyridin-2-yl]oxy}-4-isopropoxyphenyl)-N-(phenylsulfonyl)propanamide (1.02 g, yield: 52%) as white crystals. melting point 129.0-129.5° C.

### Example 5

To a solution of 3-[2-{[3-chloro-5-(trifluoromethyl)pyridin-2-yl]oxy}-4-(2-methoxyethoxy)phenyl]propionic acid (114 mg), in tetrahydrofuran (2 ml) were added thionyl chloride (0.05 ml) and N,N-dimethylformamide (0.01 ml) under ice-cooling, and the mixture was stirred for 2 hr. The reaction mixture was concentrated, and the residue was dissolved in tetrahydrofuran (2 ml). 4-Chlorobenzenesulfonamide (60 mg), N,N-diisopropylethylamine (0.05 ml) and 4-dimethylaminopyridine (39 mg) were added to the reaction mixture at room temperature, and the mixture was stirred for 3 hr. A saturated aqueous ammonium chloride solution was added to the reaction mixture, and the mixture was diluted with ethyl acetate. The organic layer was washed with saturated brine, dried (MgSO₄), filtrated and concentrated. The obtained residue was subjected to silica gel column chromatography, and eluted with ethyl acetate-hexane (1:9-2:3, v/v) to give N-[(4-chlorophenyl)sulfonyl]-3-[2-{[3-chloro-5-(trifluoromethyl)pyridin-2-yl]oxy}-4-(2-methoxyethoxy)phenyl]propanamide (88 mg, yield: 54%) as a white solid. Recrystallization from ethyl acetate-hexane gave white crystals. melting point 140-141.5° C.

### Example 6

To a solution of 3-[2-{[3-chloro-5-(trifluoromethyl)pyridin-2-yl]oxy}-4-(2-methoxyethoxy)phenyl]propionic acid (140 mg) in tetrahydrofuran (2 ml) were added thionyl chloride (0.05 ml) and N,N-dimethylformamide (0.01 ml) under ice-cooling, and the mixture was stirred for 2 hr. The reaction mixture was concentrated, and the residue was dissolved in tetrahydrofuran (2 ml). p-Toluenesulfonamide (65 mg), N,N-diisopropylethylamine (0.10 ml) and 4-dimethylaminopyridine (42 mg) were added to the reaction mixture at room temperature, and 30 min later, N,N-diisopropylethylamine (0.10 ml) was added. A saturated aqueous ammonium chloride solution was added to the reaction mixture, and the mixture was diluted with ethyl acetate. The organic layer was washed with saturated brine, dried (MgSO₄), filtrated and concentrated. The obtained residue was subjected to silica gel column chromatography, and eluted with ethyl acetate-hexane (1:9-2:3, v/v) to give a pale-yellow oil. The obtained oil was mixed with an excess amount of triethylamine, and an excess amount of the reagent was evaporated under reduced pressure. The obtained mixture was subjected to silica gel column chromatography, and eluted with ethyl acetate-hexane (1:19-4:1, v/v) to give 3-[2-{[3-chloro-5-(trifluoromethyl)pyridin-2-yl]oxy}-4-(2-methoxyethoxy)phenyl]-N-[(4-methylphenyl)sulfonyl]propanamide (23 mg, 12%) as a colorless oil.

¹H-NMR (300 MHz, CDCl₃) δ:2.42-2.51 (2H, m), 2.83 (2H, t, J=7.3 Hz), 3.44 (3H, s), 3.70-3.77 (2H, m), 4.00-4.11 (2H, m), 6.40-6.49 (2H, m), 6.82 (1H, d, J=9.1 Hz), 7.22-7.35 (2H, m), 7.72 (2H, d, J=8.5 Hz), 8.06 (1H, d, J=2.2 Hz), 8.42 (1H, s).

### Example 7

To a solution of 3-[2-{[3-chloro-5-(trifluoromethyl)pyridin-2-yl]oxy}-4-(2-methoxyethoxy)phenyl]propionic acid (169 mg) in tetrahydrofuran (2 ml) were added thionyl chloride (0.05 ml) and N,N-dimethylformamide (0.01 ml) under ice-cooling, and the mixture was stirred for 2 hr. The reaction mixture was concentrated, and the residue was dissolved in tetrahydrofuran (2 ml). 4-Methoxybenzenesulfonamide (85 mg), N,N-diisopropylethylamine (0.10 ml) and 4-dimethylaminopyridine (62 mg) were added to the reaction mixture at room temperature, and the mixture was stirred for 80 min. A saturated aqueous ammonium chloride solution was added to the reaction mixture, and the mixture was diluted with ethyl acetate. The organic layer was washed with saturated brine, dried (MgSO₄), filtrated and concentrated. The obtained residue was subjected to silica gel column chromatography, and eluted with ethyl acetate-hexane (1:9-2:3, v/v) to give a colorless oil. This was subjected to basic silica gel column chromatography, and eluted with ethyl acetate-methanol (ethyl acetate alone to 4:1, v/v), then with a 2.0 M ammonia methanol solution to give a red solid. The obtained solid was subjected to silica gel column chromatography, and eluted with ethyl acetate-hexane (1:9-2:3, v/v) to give 3-[2-{[3-chloro-5-(trifluoromethyl)pyridin-2-yl]oxy}-4-(2-methoxyethoxy)phenyl]-N-[(4-methoxyphenyl)sulfonyl]propanamide (125 mg, yield: 53%) as a colorless solid. Recrystallization from ethyl acetate-hexane gave a white powder. melting point 118-120° C.

### Example 8

To a solution of 3-[2-{[3-chloro-5-(trifluoromethyl)pyridin-2-yl]oxy}-4-(2-methoxyethoxy)phenyl]propionic acid (494 mg) in tetrahydrofuran (5 ml) were added thionyl chloride (0.15 ml) and N,N-dimethylformamide (0.05 ml) under ice-cooling, and the mixture was stirred for 2 hr. The reaction mixture was concentrated, and the residue was dissolved in tetrahydrofuran (5 ml). 2-Chlorobenzenesulfonamide (245 mg), N,N-diisopropylethylamine (0.40 ml) and 4-dimethylaminopyridine (167 mg) were added to the reaction mixture at room temperature, and the mixture was stirred for 2 hr. A saturated aqueous ammonium chloride solution was added to the reaction mixture, and the mixture was diluted with ethyl acetate. The organic layer was washed with saturated brine, dried (MgSO₄), filtrated and concentrated. The obtained residue was subjected to silica gel column chromatography, and eluted with ethyl acetate-hexane (15:85-1:1, v/v) to give a colorless oil. The obtained oil was mixed with an excess amount of triethylamine, and an excess amount of the reagent was evaporated under reduced pressure. The obtained mixture was subjected to silica gel column chromatography, and eluted with ethyl acetate-hexane (1:4-4:1, v/v) to give N-[(2-chlorophenyl)sulfonyl]-3-[2-{[3-chloro-5-(trifluoromethyl)pyridin-2-yl]oxy}-4-(2-methoxyethoxy)phenyl]propanamide (608 mg, 87%) as a white solid.

¹H-NMR (300 MHz, CDCl₃) δ:2.55 (2H, t, J=7.3 Hz), 2.78 (2H, t, J=7.3 Hz), 3.43 (3H, s), 3.70-3.76 (2H, m), 4.01-4.10 (2H, m), 6.56 (1H, d, J=2.5 Hz), 6.68 (1H, dd, J=8.5, 2.5 Hz), 7.03 (1H, d, J=8.5 Hz), 7.38-7.48 (2H, m), 7.49-7.59 (1H, m), 8.00 (1H, d, J=2.2 Hz), 8.20 (1H, dd, J=7.8, 1.8 Hz), 8.33 (1H, d, J=1.4 Hz).

### Example 9

To a solution of 3-[2-{[3-chloro-5-(trifluoromethyl)pyridin-2-yl]oxy}-4-(2-methoxyethoxy)phenyl]propionic acid (515 mg) in tetrahydrofuran (5 ml) were added thionyl chloride (0.15 ml) and N,N-dimethylformamide (0.05 ml) under ice-cooling, and the mixture was stirred for 2 hr. The reaction mixture was concentrated, and the residue was dissolved in tetrahydrofuran (5 ml). o-Toluenesulfonamide (221 mg), N,N-diisopropylethylamine (0.40 ml) and 4-dimethylaminopyridine (140 mg) were added to the reaction mixture at room temperature, and the mixture was stirred for 2 hr. A saturated aqueous ammonium chloride solution was added to the reaction mixture, and the mixture was diluted with ethyl acetate. The organic layer was washed with saturated brine, dried (MgSO₄), filtrated and concentrated. The obtained residue was subjected to silica gel column chromatography, and eluted with ethyl acetate-hexane (15:85-1:1, v/v) to give 3-[2-{[3-chloro-5-(trifluoromethyl)pyridin-2-yl]oxy}-4-(2-methoxyethoxy)phenyl]-N-[(2-methylphenyl)sulfonyl]propanamide (171 mg, yield: 24%) as a pale-yellow oil.

¹H-NMR (300 MHz, CDCl₃) δ:2.37 (3H, s), 2.50 (2H, t, J=7.2 Hz), 2.82 (2H, t, J=7.2 Hz), 3.44 (3H, s), 3.68-3.79 (2H, m), 4.03-4.10 (2H, m), 6.45-6.62 (2H, m), 6.88 (1H, d, J=8.5 Hz), 7.22-7.27 (1H, m), 7.30-7.39 (1H, m), 7.44-7.57 (1H, m), 7.97-8.11 (2H, m), 8.42 (1H, d, J=1.1 Hz), 9.19 (1H, s).

### Example 10

To a solution of 3-[2-{[3-chloro-5-(trifluoromethyl)pyridin-2-yl]oxy}-4-(2-methoxyethoxy)phenyl]propionic acid (498 mg) in tetrahydrofuran (5 ml) were added thionyl chloride (0.15 ml) and N,N-dimethylformamide (0.05 ml) under ice-cooling, and the mixture was stirred for 3 hr. The reaction mixture was concentrated, and the residue was dissolved in tetrahydrofuran (5 ml). 4-Ethylbenzenesulfonamide (249 mg), N,N-diisopropylethylamine (0.40 ml) and 4-dimethylaminopyridine (158 mg) were added to the reaction mixture at room temperature, and the mixture was stirred for 2 hr. A saturated aqueous ammonium chloride solution was added to the reaction mixture, and the mixture was diluted with ethyl acetate. The organic layer was washed with saturated brine, dried (MgSO₄), filtrated and concentrated. The obtained residue was subjected to silica gel column chromatography, and eluted with ethyl acetate-hexane (15:85-1:1, v/v) to give 3-[2-{{3-chloro-5-(trifluoromethyl)pyridin-2-yl}oxy}-4-(2-methoxyethoxy)phenyl]-N-[(4-ethylphenyl)sulfonyl]propanamide (157 mg, yield: 23%) as a white solid.

¹H-NMR (300 MHz, CDCl₃) δ:1.29 (3H, t, J=7.7 Hz), 2.45 (2H, t, J=7.1 Hz), 2.69-2.89 (4H, m), 3.44 (3H, s), 3.70-3.76 (2H, m), 4.02-4.07 (2H, m), 6.39-6.52 (2H, m), 6.82 (1H, d, J=8.0 Hz), 7.28-7.36 (2H, m), 7.70-7.81 (2H, m), 8.06 (1H, d, J=2.2 Hz), 8.37-8.49 (1H, m), 9.21 (1H, s).

### Example 11

To a solution of 3-[2-{[3-chloro-5-(trifluoromethyl)pyridin-2-yl]oxy}-4-(2-methoxyethoxy)phenyl]propionic acid (496 mg) in tetrahydrofuran (5 ml) were added thionyl chloride (0.15 ml) and N,N-dimethylformamide (0.05 ml) under ice-cooling, and the mixture was stirred for 2 hr. The reaction mixture was concentrated, and the residue was dissolved in tetrahydrofuran (5 ml). 4-Fluorobenzenesulfonamide (225 mg), N,N-diisopropylethylamine (0.40 ml) and 4-dimethylaminopyridine (140 mg) were added to the reaction mixture at room temperature, and the mixture was stirred for overnight. A saturated aqueous ammonium chloride solution was added to the reaction mixture, and the mixture was diluted with ethyl acetate. The organic layer was washed with saturated brine, dried (MgSO₄), filtrated and concentrated. The obtained residue was subjected to silica gel column chromatography, and eluted with ethyl acetate-hexane (15:85-1:1, v/v) to give a white solid. The obtained solid was mixed with an excess amount of triethylamine, and an excess amount of the reagent was evaporated under reduced pressure. The obtained mixture was subjected to silica gel column chromatography, and eluted with ethyl acetate-hexane (1:1-9:1, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]-N-[(4-fluorophenyl)sulfonyl]propanamide (105 mg, yield: 15%) as a white powder. melting point 136-138° C.

### Example 12

To a solution of (2E)-3-[2-{[3-chloro-5-(trifluoromethyl)pyridin-2-yl]oxy}-4-(2-methoxyethoxy)phenyl]acrylic acid (2.31 g) in tetrahydrofuran (50 ml) was added N,N′-carbonyldiimidazole (1.37 g), and the mixture was heated under reflux for 1 hr. After cooling to room temperature, pentane-1-sulfonamide (1.02 g) and 1,8-diazabicyclo[5.4.0]undec-7-ene (0.80 ml) were added to the reaction mixture, and the mixture was stirred for 3 hr. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:4, v/v) to give a white solid. Recrystallization from ethyl acetate-hexane gave (2E)-3-[2-{[3-chloro-5-(trifluoromethyl)pyridin-2-yl]oxy}-4-(2-methoxyethoxy)phenyl]-N-(pentylsulfonyl)acrylamide (493 mg, yield: 16%) as white crystals. melting point 127-130° C.

¹H-NMR (300 MHz, CDCl₃) δ:0.89 (3H, t, J=7.1 Hz), 1.24-1.48 (4H, m), 1.83 (2H, tt, J=7.6, 7.6 Hz), 3.44 (3H, s), 3.45-3.50 (2H, m), 3.71-3.80 (2H, m), 4.04-4.20 (2H, m), 6.37 (1H, d, J=15.6 Hz), 6.70 (1H, d, J=2.4 Hz), 6.91 (1H, dd, J=8.7, 2.4 Hz), 7.60 (1H, d, J=8.9 Hz), 7.79 (1H, d, J=15.6 Hz), 7.87 (1H, s), 8.03 (1′H, d, J=2.1 Hz), 8.25 (1H, dd, J=2.2, 1.0 Hz).

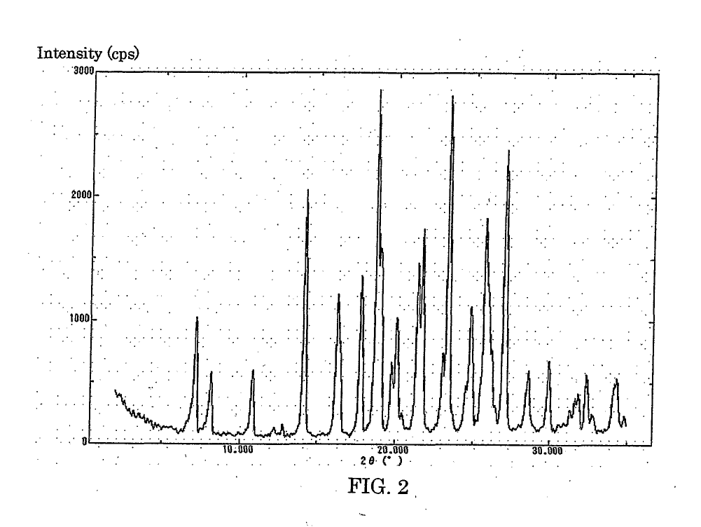

Recrystallization of the crude crystals obtained under the same conditions as in Example 12 from ethanol gave crystals. The X-ray powder diffraction pattern of this crystal as measured using Cu-Kα ray (tube voltage: 40 KV; tube current: 50 mA) as a radiation source and RINT2100 type Ultima+(Rigaku Corporation), is shown in FIG. 2.

Data (main peak) of X-ray powder diffraction

| diffraction angle: 2θ (°) | spacing: d value (angstrom) |
|---|---|
| 7.18 | 12.3 |
| 14.2 | 6.27 |
| 16.3 | 5.44 |
| 17.8 | 4.99 |
| 18.7 | 4.74 |
| 21.7 | 4.09 |
| 23.3 | 3.81 |
| 25.7 | 3.46 |
| 27.0 | 3.29 |

### Example 13

To a solution of (2E)-3-[2-{[3-chloro-5-(trifluoromethyl)pyridin-2-yl]oxy}-4-isopropoxyphenyl]acrylic acid (0.95 g) in tetrahydrofuran (20 ml) was added N,N′-carbonyldiimidazole (628 mg), and the mixture was heated under reflux for 1.5 hr. After cooling to room temperature, pentane-1-sulfonamide (397 mg) and 1,8-diazabicyclo[5.4.0]undec-7-ene (0.50 ml) were added to the reaction mixture, and the mixture was stirred overnight. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give a white solid. Recrystallization from ethyl acetate-hexane gave (2E)-3-[2-{[3-chloro-5-(trifluoromethyl)pyridin-2-yl]oxy}-4-isopropoxyphenyl]-N-(pentylsulfonyl)acrylamide (438 mg, yield: 35%) as white fine needles. melting point 142-144° C.

### Example 14

To a solution of 3-[2-{[3-chloro-5-(trifluoromethyl)pyridin-2-yl]oxy}-4-(2-methoxyethoxy)phenyl]propane-1-amine (79 mg) in acetonitrile (5 ml) was added benzenesulfonylisocyanate (105 mg), and the mixture was stirred overnight at room temperature. The reaction mixture was concentrated, and the obtained residue was subjected to silica gel column chromatography, and eluted with ethyl acetate-hexane (hexane alone to 3:2, v/v) to give a white solid. The solid was recrystallized from ethyl acetate-hexane and the obtained solid was washed with acetonitrile, subjected to basic silica gel column chromatography and eluted with 2.0 M ammonia methanol solution to give a red solid. The obtained residue was subjected to silica gel column chromatography, and eluted with ethyl acetate-hexane (1:9-1:4, v/v) to give N—[({3-[2-{[3-chloro-5-(trifluoromethyl)pyridin-2-yl]oxy}-4-(2-methoxyethoxy)phenyl]propyl}amino) carbonyl]benzenesulfonamide (15 mg, yield: 13%, purity 90%) as a pale-yellow oil.

¹H-NMR (300 MHz, CDCl₃) δ:1.67-1.86 (2H, m), 2.43 (2H, t, J=7.6 Hz), 3.19 (2H, d, J=6.0 Hz), 3.43 (3H, s), 3.73 (2H, dd, J=5.5, 4.0 Hz), 4.09 (2H, dd, J=5.4, 3.9 Hz), 6.57 (1H, t, J=5.6 Hz), 6.67 (1H, t, J=, 2.6 Hz), 6.81 (1H, dd, J=8.5, 2.6 Hz), 7.13 (1H, d, J=8.5 Hz), 7.40-7.53 (2H, m), 7.60 (1H, t, J=7.5 Hz), 7.78-7.93 (2H, m), 7.99 (1H, d, J=1.9 Hz), 8.25 (1H, dd, J 2.2, 1.0 Hz).

### Example 15

To a solution of 3-[2-{[3-chloro-5-(trifluoromethyl)pyridin-2-yl]oxy}-4-(2-methoxyethoxy)phenyl]propane-1-amine (492 mg) in ethyl acetate (20 ml) were added triethylamine (0.20 ml) and methanesulfonyl chloride (0.25 ml) and the mixture was stirred at room temperature for 30 min. A saturated aqueous ammonium chloride solution was added to the reaction mixture, and the mixture was diluted with ethyl acetate. The organic layer was washed with water, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give a pale-orange solid. Recrystallization from ethyl acetate-hexane gave N-{3-[2-{[3-chloro-5-(trifluoromethyl)pyridin-2-yl]oxy}-4-(2-methoxyethoxy)phenyl]propyl}methanesulfonamide (211 mg, yield: 36%) as white crystals. melting point 89-90° C.

### Example 16

To a solution of 3-[2-{[3-chloro-5-(trifluoromethyl)pyridin-2-yl]oxy}-4-(2-methoxyethoxy)phenyl]propane-1-amine (455 mg) in ethyl acetate (10 ml) were added pyridine (1.0 ml) and benzenesulfonyl chloride (0.15 ml), and the mixture was stirred at room temperature for 30 min. A saturated aqueous ammonium chloride solution was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give a colorless solid. Recrystallization from ethyl acetate-hexane gave N-{3-[2-{[3-chloro-5-(trifluoromethyl)pyridin-2-yl]oxy}-4-(2-methoxyethoxy)phenyl]propyl}benzenesulfonamide (205 mg, yield: 34%) as white crystals. melting point 94-95° C.

### Example 17

To a solution of 3-[2-{[3-chloro-5-(trifluoromethyl)pyridin-2-yl]oxy}-4-(2-methoxyethoxy)phenyl]propane-1-amine (451.1 mg) in ethyl acetate (10 ml) were added pyridine (1.0 ml) and 2,4-dichlorobenzenesulfonyl chloride (301 mg), and the mixture was stirred at room temperature for 30 min. A saturated aqueous ammonium chloride solution was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give a pale-yellow solid. The obtained solid was subjected to reversed-phase high performance liquid chromatography, and eluted with acetonitrile-water (2:3 to acetonitrile alone, v/v) to give 2,4-dichloro-N-{3-[2-{[3-chloro-5-(trifluoromethyl)pyridin-2-yl]oxy}-4-(2-methoxyethoxy)phenyl]propyl}benzenesulfonamide (236 mg, yield: 35%) as a colorless oil.

¹H-NMR (300 MHz, CDCl₃) δ:1.68-1.82 (2H, m), 2.46 (2H, t, J 7.3 Hz), 2.75-2.96 (2H, m), 3.44 (3H, s), 3.65-3.78 (2H, m), 4.00-4.15 (2H, m), 4.99 (2H, t, J=6.1 Hz), 6.65 (1H, d, J=2.4 Hz), 6.80 (1H, dd, J=8.5, 2.4 Hz), 7.11 (1H, d, J=8.5 Hz), 7.37 (1H, dd, J=8.5, 1.9 Hz), 7.51 (1H, d, J=1.9 Hz), 7.90-8.03 (2H, m), 8.25 (1H, d, J=0.9 Hz).

### Example 18

To a solution of 3-[2-{[3-chloro-5-(trifluoromethyl)pyridin-2-yl]oxy}-4-(27 methoxyethoxy)phenyl]propane-1-amine (1.01 g) in ethyl acetate (10 ml) were added pyridine (2.5 ml) and 1-pentanesulfonyl chloride (0.50 g), and the mixture was stirred at room temperature for 1.5 hr. A saturated aqueous ammonium chloride solution was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give a white solid. Recrystallization from ethyl acetate-hexane gave N-{3-[2-{[3-chloro-5-(trifluoromethyl)pyridin-2-yl]oxy}-4-(2-methoxyethoxy)phenyl]propyl}pentane-1-sulfonamide (198 mg, yield: 15%) as white crystals. melting point 87-90° C.

### Example 19

To a solution of 3-[2-{[3-chloro-5-(trifluoromethyl)pyridin-2-yl]oxy}-4-(2-methoxyethoxy)phenyl]propane-1-amine (289 mg) in ethyl acetate (10 ml) were added pyridine (5.0 ml) and phenylmethanesulfonyl-chloride (182.1 mg), and the mixture was stirred at room temperature for 2.5 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give a white solid. Recrystallization from ethyl acetate-hexane gave N-{3-[2-{[3-chloro-5-(trifluoromethyl)pyridin-2-yl]oxy}-4-(2-methoxyethoxy)phenyl]propyl}-1-phenylmethanesulfonamide (75 mg, yield: 19%) as white crystals. melting point 97-100° C.

### Example 20

To a solution of 3-[2-{[3-chloro-5-(trifluoromethyl)pyridin-2-yl]oxy}-4-(2-methoxyethoxy)phenyl]propane-1-amine (505 mg) in ethyl acetate (10 ml) were added pyridine (0.5 ml) and cyclohexanesulfonyl chloride (362 mg), and the mixture was stirred at room temperature for 3.5 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give N-{3-[2-{[3-chloro-5-(trifluoromethyl)pyridin-2-yl]oxy}-4-(2-methoxyethoxy)phenyl]propyl}cyclohexanesulfonamide (53 mg, yield: 8%) as a pale-yellow oil.

¹H-NMR (300 MHz, CDCl₃) δ:1.11-1.94 (10H, m), 1.99-2.18 (2H, m), 2.53 (2H, t, J=7.3 Hz), 2.66-2.94 (2H, m), 3.08 (2H, m), 3.44 (3H, s), 3.68-3.80 (2H, m), 3.96-4.15 (2 H, m), 6.67 (1H, d, J=2.6 Hz), 6.84 (1H, dd, J=8.5, 2.4 Hz), 7.20 (1H, d, J=8.5 Hz), 8.00 (1H, d, J=2.1 Hz), 8.27 (1H, d, J=0.8 Hz).

### Example 21

To a solution of 3-[2-{[3-chloro-5-(trifluoromethyl)pyridin-2-yl]oxy}-4-(2-methoxyethoxy)phenyl]propane-1-amine (196 mg) in ethyl acetate (10 ml) were added pyridine (0.5 ml) and 3-phenylpropanesulfonyl chloride (182 mg), and the mixture was stirred at room temperature for 1.5 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give a white solid. The obtained solid was dissolved in a suspension of activated carbon in ethyl acetate, and the mixture was stirred for 3 hr, filtrated and concentrated to give a white solid. Recrystallization from ethyl acetate-hexane gave N-{3-[2-{[3-chloro-5-(trifluoromethyl)pyridin-2-yl]oxy}-4-(2-methoxyethoxy)phenyl]propyl}-3-phenylpropane-1-sulfonamide (75 mg, yield: 26%) as white crystals. melting point 101.8-102.0° C.

### Example 22

To a solution of 3-[2-{[3-chloro-5-(trifluoromethyl)pyridin-2-yl]oxy}-4-(2-methoxyethoxy)phenyl]propane-1-amine (234 mg) in ethyl acetate (10 ml) were added pyridine (5.0 ml) and 4-; chlorobenzenesulfonyl chloride (185 mg), and the mixture was stirred at room temperature for 4 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-35:65, v/v). Activated carbon was suspended in the eluted fraction, and the mixture was stirred for 1 hr, filtrated and concentrated to give a white solid. Recrystallization from ethyl acetate-hexane gave 4-chloro-N-{3-[2-{[3-chloro-5-(trifluoromethyl)pyridin-2-yl]oxy}-4-(2-methoxyethoxy)phenyl]propyl}benzenesulfonamide (145 mg, yield: 43%) as white feather crystals. melting point 111-113° C.

### Example 23

To a solution of 3-[2-{[3-chloro-5-(trifluoromethyl)pyridin-2-yl]oxy}-4-(2-methoxyethoxy)phenyl]propane-1-amine (275 mg) in ethyl acetate (10 ml) were added pyridine (5.0 ml) and 2-chlorobenzenesulfonyl chloride (227 mg), and the mixture was stirred at room temperature for 6 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give a pale-yellow oil. The obtained oil was dissolved in a suspension of activated carbon in ethyl acetate, and the mixture was stirred for 1 hr. The reaction mixture was filtrated and concentrated to give 2-chloro-N-{3-[2-{[3-chloro-5-(trifluoromethyl)pyridin-2-yl]oxy}-4-(2-methoxyethoxy)phenyl]propyl}benzenesulfonamide (10 mg, yield: 3%) as a pale-yellow oil.

¹H-NMR (300 MHz, CDCl₃) δ:1.63-1.81 (10H, m), 2.46 (2H, t, J=7.3 Hz), 2.89 (2H, q, J=6.7 Hz), 3.43 (3H, s), 3.69-3.76 (2H, m), 4.01-4.12 (2H, m), 5.00 (1H, t, J=6.0 Hz), 6.64 (1H, d, J=2.4 Hz), 6.79 (1H, dd, J=8.6, 2.5 Hz), 7.11 (1H, d, J=8.5 Hz), 7.30-7.45 (1H, m), 7.50 (2H, d, J=3.8 Hz), 7.98 (1H, d, J=1.9 Hz), 8.06 (1H, d, J=7.7 Hz), 8.24 (1H, s).

### Example 24

To a solution of 3-[2-{[3-chloro-5-(trifluoromethyl)pyridin-2-yl]oxy}-4-(2-methoxyethoxy)phenyl]propane-1-amine (150 mg) in ethyl acetate (10 ml) were added pyridine (5.0 ml) and 3-chlorobenzenesulfonyl chloride (191 mg), and the mixture was stirred at room temperature for 4 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give a white solid. Recrystallization from ethyl acetate-hexane gave 3-chloro-N-{3-[2-{[3-chloro-5-(trifluoromethyl)pyridin-2-yl]oxy}-4-(27 methoxyethoxy)phenyl]propyl}benzenesulfonamide (40 mg, yield: 19%) as white feather crystals. melting point 110-114° C.

### Example 25

To a solution of 3-[2-{[3-chloro-5-(trifluoromethyl)pyridin-2-yl]oxy}-4-(2-methoxyethoxy)phenyl]propane-1-amine (147 mg) in ethyl acetate (10 ml) were added pyridine (5.0 ml) and 2-propanesulfonyl chloride (122 mg), and the mixture was stirred at room temperature for 6 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, 7/v). Activated carbon was suspended in the eluted fraction, and the mixture was stirred for 1 hr. The reaction mixture was filtrated and concentrated to give N-{3-[2-{[3-chloro-5-(trifluoromethyl)pyridin-2-yl]oxy}-4-(2-methoxyethoxy)phenyl]propyl}propane-2-sulfonamide (11 mg, yield: 6%) as a pale-yellow oil.

¹H-NMR (300 MHz, CDCl₃) δ:1.32 (6H, d, J=6.8 Hz), 1.61-2.07 (2H, m), 2.53 (2H, t, J=7.3 Hz), 2.88-2.99 (1H, m), 3.01-3.22 (2H, m), 3.38-3.48 (3H, m), 3.64-3.76 (2H, m), 3.95-4.17 (2H, m), 6.67 (1H, d, J=2.4 Hz), 6.84 (1H, dd, J=8.5, 2.6 Hz), 7.20 (1H, d, J=8.5 Hz), 8.00 (1H, s), 8.27 (1H, d, J=1.1 Hz).

### Example 26

A solution of (2E)-3-[1-[2-chloro-4-(trifluoromethyl)benzyl]-3-(cyclopropylmethoxy)-1H-pyrazol-5-yl]acrylic acid (150 mg) and N,N′-carbonyldiimidazole (67 mg) in N,N-dimethylformamide (5 ml) was stirred at room temperature for 1 hr. To this mixture were added 3-methylbutane-1-sulfonamide (62 mg) and 1,8-diazabicyclo[5.4.0]undec-7-ene (0.062 ml), and the mixture was stirred at 100° C. for 12, hr. After cooling to room temperature, 1N hydrochloric acid was added to the reaction mixture, and the mixture was adjusted to about pH 4. Water was added to the mixture, and the mixture was extracted with ethyl acetate. The organic layer was washed with saturated brine, dried (MgSO₄), filtrated and concentrated. The residue was subjected to silica gel column chromatography, eluted with ethyl acetate-hexane (1:20-2:3, v/v) and concentrated. The obtained crude crystals were recrystallized from ethyl acetate-hexane to give (2E)-3-[1-[2-chloro-4-(trifluoromethyl)benzyl]-3-(cyclopropylmethoxy)-1H-pyrazol-5-yl]-N-[(3-methylbutyl)sulfonyl]acrylamide (124 mg, yield: 62%) as colorless crystals. melting point 155.5-157.5° C.

### Example 27

To a solution of (2E)-3-[2-{[3-chloro-5-(trifluoromethyl)pyridin-2-yl]oxy}-4-(2-methoxyethoxy)phenyl]-2-propen-1-ol (2.26 g) in ethyl acetate (40 ml) were added triethylamine (3.0 ml) and methanesulfonyl chloride (1.2 ml), and the mixture was stirred at room temperature for 2 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated to give an orange oil. To a solution of the obtained oil in N,N-dimethylformamide (30 ml) was added potassium phthalimide (1.02 g), and the mixture was stirred at 80° C. for 2 hr. After cooling to room temperature, water was added to the reaction mixture, and the mixture was extracted with ethyl acetate. The organic layer was washed with water and saturated brine, dried (MgSO₄), filtrated and concentrated. The obtained solid was washed with diisopropyl ether and hexane to give a brown solid. To a solution of the obtained solid in methanol (100 ml) was added hydrazine monohydrate (2.19 g), and the mixture was stirred at 50° C. for 1 hr. The reaction mixture was concentrated, the residue was washed with diethyl ether, and the filtrate was concentrated. The obtained residue was dissolved in ethyl acetate, and the organic layer was washed with saturated aqueous sodium hydrogencarbonate solution and saturated brine, dried (MgSO₄), filtrated and concentrated to give (2E)-3-[2-{[3-chloro-5-(trifluoromethyl)pyridin-2-yl]oxy}-4-(2-methoxyethoxy)phenyl]-2-propene-1-amine (1.30 g, yield: 63%) as a brown oil.

To a solution of the obtained oil (480 mg) in ethyl acetate (10 ml) were added pyridine (0.50 ml) and 1-pentanesulfonyl chloride (240 mg), and the mixture was stirred at room temperature for 30 min. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give a white solid. Recrystallization from ethyl acetate-hexane gave N-{(2E)-3-[2-{[3-chloro-5-(trifluoromethyl)pyridin-2-yl]oxy}-4-(2-methoxyethoxy)phenyl]-2-propen-1-yl}pentane-1-sulfonamide (46 mg, yield: 7%) as white crystals. melting point 106.0-106.5° C.

### Example 28

To a solution of triphosgene (68 mg) in dichloromethane (5 ml) were added 3-[2-{[3-chloro-5-(trifluoromethyl)pyridin-2-yl]oxy}-4-(2-methoxyethoxy)phenyl]propan-1-ol (205 mg) and pyridine (0.10 ml) under ice-cooling, and the reaction mixture was immediately concentrated. To a solution of the obtained residue in tetrahydrofuran (10 ml) were added pentane-1-sulfonamide (125 mg), N,N-diisopropylethylamine (0.40 ml) and 4-dimethylaminopyridine (62 mg), and the mixture was stirred at room temperature for 10 min. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]propyl (pentylsulfonyl)carbamate (35 mg, yield: 12%) as a white powder. melting point 97.5-99.0° C.

¹H-NMR (300 MHz, CDCl₃) δ:0.91 (3H, t, J=7.2 Hz), 1.21-1.52 (4H, m), 1.71-1.90 (2H, m), 1.89-2.05 (2H, m), 2.58 (2H, t, J=7.3 Hz), 3.29-3.42 (2H, m), 3.43 (3H, s), 3.69-3.75 (2H, m), 4.04-4.12 (2H, m), 4.16 (2H, t, J=6.3 Hz), 6.68 (1H, d, J=2.4 Hz), 6.83 (1H, dd, J=8.7, 2.4 Hz), 7.19 (1H, d, J=8.5 Hz), 7.34 (1H, s), 8.02 (1H, d, J=2.1 Hz), 8.28 (1H, d, J=1.1 Hz).

### Example 29

tert-Butyl {3-[2-{[3-chloro-57 (trifluoromethyl)pyridin-2-yl]oxy}-4-(2-methoxyethoxy)phenyl]propyl}[(2-nitrophenyl)sulfonyl]carbamate (3.44 g) was dissolved in a 4N hydrochloric acid ethyl acetate solution (40 ml), and the mixture was stirred at room temperature for 3 hr and at 40° C. for 1 hr. After cooling to room temperature, concentrated hydrochloric acid (2.0 ml) was added to the reaction mixture, and the mixture was stirred for 1 hr. Trifluoroacetic acid (4.0 ml) was added, and the mixture was further stirred for 2 hr. A saturated aqueous sodium hydrogencarbonate solution was added to the reaction mixture, and the mixture was diluted with ethyl acetate. The organic layer was washed with water, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-3:7, v/v) to give N-{3-[2-{[3-chloro-5-(trifluoromethyl)pyridin-2-yl]oxy}-4-(2-methoxyethoxy)phenyl]propyl}-2-nitrobenzenesulfonamide (2.77 g, yield: 95%) as a white solid. Recrystallization from ethyl acetate-hexane gave white feather crystals. melting point 125.5-126.0° C.

### Example 30

To a solution of 3-[2-{[3-chloro-5-(trifluoromethyl)pyridin-2-yl]oxy}-4-(2-methoxyethoxy)phenyl]propan-1-ol (400 mg) in acetonitrile (10 ml) were added N,N-diisopropylethylamine (0.40 ml) and p-toluenesulfonylisocyanate (202 mg), and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/v) to give a colorless oil. The obtained oil was dissolved in ethyl acetate, acetic anhydride (0.5 ml) and pyridine (0.5 ml) were added, and the mixture was stirred at room temperature for 2 hr. A saturated aqueous sodium hydrogencarbonate solution was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-2:3, v/v) to give a colorless oil. The obtained oil was subjected to reversed-phase high performance liquid chromatography, and eluted with acetonitrile-water (2:3 to acetonitrile alone, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]propyl [(4-methylphenyl)sulfonyl]carbamate monohydrate (105 mg, yield: 18%) as white crystals. melting point 118-120° C.

### Example 31

To a solution of 3-[2-{[3-chloro-5-(trifluoromethyl)pyridin-2-yl]oxy}-4-(2-methoxyethoxy)phenyl]propan-1-ol (402 mg) in acetonitrile (5 ml) were added N,N-diisopropylethylamine (0.40 ml) and benzenesulfonylisocyanate (190 mg), and the mixture was stirred at room temperature for 1 hr. Acetic anhydride (0.5 ml) and pyridine (0.5 ml) were added, and the mixture was stirred at room temperature for 1 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained-residue was subjected to silica gel column chromatography, and eluted with ethyl acetate-hexane (1:19-2:3, v/v) to give 3-[2-{[3-chloro-5-(trifluoromethyl)pyridin-2-yl]oxy}-4-(2-methoxyethoxy)phenyl]propyl (phenylsulfonyl)carbamate (264.8 mg, yield: 45%) as a colorless oil. The oil was subjected to reversed-phase high performance liquid chromatography, and eluted with acetonitrile-water (2:3 to acetonitrile alone, v/v) to give a white solid. Recrystallization from ethyl acetate-diisopropyl ether gave white crystals as 1.5 hydrate. melting point 174-177° C.

### Example 32

To a solution of 3-[2-{[3-chloro-5-(trifluoromethyl)pyridin-2-yl]oxy}-4-(2-methoxyethoxy)phenyl]propan-1-ol (397 mg) in acetonitrile (5 ml) were added N,N-diisopropylethylamine (0.40 ml) and 4-chlorobenzenesulfonylisocyanate (218 mg), and the mixture was stirred at room temperature for 2 hr. Acetic anhydride (0.5 ml) and pyridine (0.5 ml) were added, and the mixture was stirred at room temperature for 1 hr. A saturated aqueous sodium hydrogencarbonate solution was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give a colorless solid. Recrystallization from ethyl acetate-hexane gave 3-[2-{[3-chloro-5-(trifluoromethyl)pyridin-2-yl]oxy}-4-(2-methoxyethoxy)phenyl]propyl [(4-chlorophenyl)sulfonyl]carbamate (161 mg, yield: 26%) as white crystals. melting point 147-150° C.

### Example 33

To a solution of triphosgene (130 mg) in toluene (10 ml) were added 3-[2-{[3-chloro-5-(trifluoromethyl)pyridin-2-yl]oxy}-4-(2-methoxyethoxy)phenyl]propan-1-ol (399.8 mg) and pyridine (0.10 ml) under ice-cooling, and the mixture was stirred for 5 min. 2-Chlorobenzenesulfonamide (208 mg), N,N-diisopropylethylamine (0.40 ml), 4-dimethylaminopyridine (148 mg) and tetrahydrofuran (30 ml) were added to the reaction mixture, and the mixture was stirred overnight at room temperature. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, eluted with ethyl acetate-hexane (1:9-1:1, v/v), and then subjected to reversed-phase high performance liquid chromatography, and eluted with acetonitrile-water (2:3 to acetonitrile alone, v/v) to give 3-[2-{[3-chloro-5-(trifluoromethyl)pyridin-2-yl]oxy}-4-(2-methoxyethoxy)phenyl]propyl [(2-chlorophenyl)sulfonyl]carbamate (216 mg, yield: 35%) as a colorless amorphous form.

¹H-NMR (300 MHz, CDCl₃) δ:1.61-1.75 (2H, m), 2.30 (2H, t, J=7.0 Hz), 3.41 (3H, s), 3.62-3.73 (2H, m), 3.76-3.86 (2H, m), 3.97-4.07 (2H, m), 6.60 (1H, d, J=2.4 Hz), 6.68 (1H, dd, J=8.4, 2.4 Hz), 7.00 (1H, d, J=8.5 Hz), 7.17 (1H, s), 7.30 (2H, s), 7.92 (1H, d, J=2.1 Hz), 8.07 (1H, d, J=7.9 Hz), 8.21 (1H, d, J=1.1 Hz).

### Example 34

To a solution of triphosgene (136 mg) in toluene (10 ml) were added 3-[2-{[3-chloro-5-(trifluoromethyl)pyridin-2-yl]oxy}-4-(2-methoxyethoxy)phenyl]propan-1-ol (396.8 mg) and pyridine (0.10 ml) under ice-cooling, and the mixture was stirred, for 5 min. o-Toluenesulfonamide (181 mg), N,N-diisopropylethylamine (0.40 ml), 4-dimethylaminopyridine (142 mg) and tetrahydrofuran (30 ml) were added to the reaction mixture, and the mixture was stirred overnight at room temperature. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/v), and then subjected to reversed-phase high performance liquid chromatography, and eluted with acetonitrile-water (2:3 to acetonitrile alone, v/v) to give 3-[2-{[3-chloro-5-(trifluoromethyl)pyridin-2-yl]oxy}-4-(2-methoxyethoxy)phenyl]propyl [(2-methylphenyl)sulfonyl]carbamate (200 mg, yield: 34%) as a colorless amorphous form.

¹H-NMR (300 MHz, CDCl₃) δ:1.59-1.83 (2H, m), 2.27-2.41 (2H, m), 2.56 (3H, s), 3.41 (3H, s), 3.60-3.76 (2H, m), 3.76-3.91 (2H, m), 3.98-4.05 (2H, m), 6.61 (1H, d, J=2.4 Hz), 6.67 (1H, d, J=9.0 Hz), 6.96-7.22 (3H, m), 7.32 (1H, d, J=5.5 Hz), 7.92 (1H, d, J=1.3 Hz), 7.97 (1H, d, J=7.3 Hz), 8.20 (1H, s).

### Example 35

To a solution of triphosgene (122 mg) in toluene (10 ml) were added 3-[2-{[3-chloro-5-(trifluoromethyl)pyridin-2-yl]oxy}-4-(2-methoxyethoxy)phenyl]propan-1-ol (386 mg) and pyridine (0.10 ml) under ice-cooling, and the mixture was stirred for 5 min. Butane-1-sulfonamide (142 mg), N,N-diisopropylethylamine (0.40 ml), 4-dimethylaminopyridine (122 mg) and tetrahydrofuran (30 ml) were added to the reaction mixture, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, V/v), and then subjected to reversed-phase high performance liquid chromatography, and eluted with acetonitrile-water (2:3 to acetonitrile alone, v/v) to give 3-[2-{[3-chloro-5-(trifluoromethyl)pyridin-2-yl]oxy}-4-(2-methoxyethoxy)phenyl]propyl (butylsulfonyl)carbamate monohydrate (54 mg, yield: 10%) as white crystals. Recrystallization from ethyl acetate-hexane gave white crystals. melting point 94.0-96.0° C.

### Example 36

To a solution of triphosgene (142 mg) in toluene (10 ml) were added (2E)-3-[2-{[3-chloro-5-(trifluoromethyl)pyridin-2-yl]oxy}-4-(2-methoxyethoxy)phenyl]-2-propen-1-ol (388 mg) and pyridine (90 μl) under ice-cooling, and the mixture was stirred for 5 min. Pentane-1-sulfonamide (194.5 mg), N,N-diisopropylethylamine (0.40 ml), 4-dimethylaminopyridine (125 mg) and tetrahydrofuran (30 ml) were added to the reaction mixture, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9 to ethyl acetate alone, v/v), and then subjected to reversed-phase high performance liquid chromatography, and eluted with acetonitrile-water (2:3 to acetonitrile alone, v/v) to give (2E)-3-[2-{[3-chloro-5-(trifluoromethyl)pyridin-2-yl]oxy}-4-(2-methoxyethoxy)phenyl]-2-propen-1-yl (pentylsulfonyl)carbamate (14 mg, yield: 3%) as a white amorphous form.

¹H-NMR (300 MHz, CDCl₃) δ:0.90 (3H, t, J=7.1 Hz), 1.24-1.50 (4H, m), 1.67-1.95 (2H, m), 3.33-3.42 (2H, m), 3.44 (3H, s), 3.71-3.79 (2H, m), 4.05-4.16 (2H, m), 4.74 (2H, dd, J=6.7, 1.0 Hz), 5.97-6.31 (1H, m), 6.55-6.73 (2H, m), 6.88 (1H, dd, J=8.7, 2.4 Hz), 7.52 (1H, d, J=8.7 Hz), 8.01 (1H, d, J=1.9 Hz), 8.25 (1H, dd, J=2.1, 0.9 Hz).

### Example 37

To a solution of (2E)-3-[2-{[3-chloro-5-(trifluoromethyl)pyridin-2-yl]oxy}-4-(2-methoxyethoxy)phenyl]-2-propen-1-ol (184 mg) in acetonitrile (2.5 ml) were added N,N-diisopropylethylamine (0.40 ml) and 4-chlorobenzenesulfonylisocyanate (140 ml), and the mixture was stirred at room temperature for 30 min. 4-Chlorobenzenesulfonylisocyanate (90 μl) was further added, and the mixture was stirred for 10 min. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained oil was subjected to reversed-phase high performance liquid chromatography, and eluted with acetonitrile-water (2:3 to acetonitrile alone, v/v) to give a white solid. Recrystallization from ethyl acetate-hexane gave (2E)-3-[2-{[3-chloro-5-(trifluoromethyl)pyridin-2-yl]oxy}-4-(2-methoxyethoxy)phenyl]-2-propen-1-yl [(4-chlorophenyl)sulfonyl]carbamate dihydrate (147 mg, yield: 52%) as white crystals. melting point 174.5° C. (dec.).

### Example 38

To a solution of 3-[2-{[3-chloro-5-(trifluoromethyl)pyridin-2-yl]oxy}-4-(2-methoxyethoxy)phenyl]propionic acid (1.90 g) in toluene (25 ml) were added triethylamine (0.90 ml) and diphenylphosphorylazide (1.40 ml), and the mixture was stirred at 80° C. for 1 hr. Benzyl alcohol (0.80 ml) was added, and the mixture was stirred with heating under reflux for 10 hr. After cooling to room temperature, the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (hexane alone to 2:3, v/v) to give benzyl {2-[2-{[3-chloro-5-(trifluoromethyl)pyridin-2-yl]oxy}-4-(2-methoxyethoxy)phenyl]ethyl}carbamate (2.02 g, yield: 89%) as a white solid. Recrystallization from ethyl acetate-hexane gave white crystals. melting point 102.5-103.0° C.

To a solution of benzyl {2-[2-{[3-chloro-5-(trifluoromethyl)pyridin-2-yl]oxy}-4-(2-methoxyethoxy)phenyl]ethyl}carbamate (450 mg) in tetrahydrofuran (5 ml) was added 10% palladium-carbon (92 mg), and the mixture was stirred under a hydrogen atmosphere at room temperature for 1 hr. The reaction mixture was filtrated and the filtrate was concentrated to give a brown oil.

The obtained oil was dissolved in ethyl acetate (5 ml), pyridine (1 ml) and 1-pentanesulfonyl chloride (227 mg) were added, and the mixture was stirred at room temperature for 1 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (hexane alone to 1:1, v/v) to give N-{3-[2-{[3-chloro-5-(trifluoromethyl)pyridin-2-yl]oxy}-4-(2-methoxyethoxy)phenyl]ethyl}pentane-1-sulfonamide (64 mg, yield: 14%) as a colorless oil.

¹H-NMR (300 MHz, CDCl₃) δ:0.73-0.94 (3H, m), 1.15-1.38 (4H, m), 1.46-1.77 (2H, m), 2.59-2.97 (4H, m), 3.18-3.33 (2H, m), 3.43 (3H, s), 3.60-3.78 (2H, m), 3.97-4.14 (2H, m), 4.55 (1H, t, J=5.8 Hz), 6.43-6.71 (1H, m), 6.84 (1H, dd, J=8.5, 2.6 Hz), 7.08 (1H, d, J=8.7 Hz), 7.94 (1H, dd, J=8.7, 2.3 Hz), 8.43 (1H, s).

### Example 39

To a solution of 3-[1-(2,4-dichlorobenzyl)-3-isopropoxy-1H-pyrazol-5-yl]propane-1-amine (0.33 g) in acetonitrile (8 ml) was added benzenesulfonylisocyanate (0.14 ml), and the mixture was stirred overnight at room temperature. The precipitated crystals were collected by filtration, washed with acetonitrile and dried (MgSO₄) to give N-[({3-[1-(2,4-dichlorobenzyl)-3-isopropoxy-1H-pyrazol-5-yl]propyl}amino)carbonyl]benzenesulfonamide (0.29 g, yield: 57%) as colorless crystals. melting point 149° C.

### Example 40

To a solution of 3-[2-{[3-chloro-5-(trifluoromethyl)pyridin-2-yl]oxy}-4-(2-methoxyethoxy)phenyl]butan-1-ol (1.00 g) in ethyl acetate (20 ml) were added triethylamine (1.20 ml) and methanesulfonyl chloride (0.70 ml), and the mixture was stirred at room temperature. After 1 hr, triethylamine (1.20 ml) and methanesulfonyl chloride (0.50 ml) were added, and the mixture was further stirred at room temperature for 30 min. The reaction mixture was diluted with ethyl acetate, and the organic layer was washed with 1N hydrochloric acid, a saturated aqueous sodium hydrogencarbonate solution and saturated brine, dried (MgSO₄), filtrated and concentrated to give an orange oil.

To a solution of pentane-1-sulfonamide (679 mg) in N,N-dimethylformamide (10 ml) was added sodium hydride (60% in oil, 198 mg) under ice-cooling, and the mixture was stirred for 30 min. To the reaction mixture was added dropwise a solution of the orange oil obtained earlier in N,N-dimethylformamide (20 ml), and the mixture was stirred at 45° C. for 60 hr. After cooling to room temperature, a saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:19-3:7, v/v) to give N-{3-[2-{[3-chloro-5-(trifluoromethyl)pyridin-2-yl]oxy}-4-(2-methoxyethoxy)phenyl]butyl}pentane-1-sulfonamide (238 mg, yield: 18%) as a yellow oil.

¹H-NMR (300 MHz, CDCl₃) δ:0.89 (3H, t, J=7.1 Hz), 1.19 (3H, d, J=7.0 Hz), 1.26-1.44 (4H, m), 1.63-2.00 (4H, m), 2.81-3.23 (5H, m), 3.43 (3H, s), 3.69-3.77 (2H, m), 4.04-4.13 (2H, m), 4.35-4.45 (1H, m), 6.61 (1H, d, J=2.6 Hz), 6.90 (1H, dd, J=8.7, 2.6 Hz), 7.23 (1H, d, J=8.7 Hz), 8.01 (1H, d, J=2.3 Hz), 8.29 (1H, d, J=0.9 Hz).

### Example 41

To a solution of 3-[2-{[3-chloro-5-(trifluoromethyl)pyridin-2-yl]oxy}-4-(2-methoxyethoxy)phenyl]-2-methylpropan-1-ol (1.22 g) in ethyl acetate (50 ml) were added triethylamine (0.80 ml) and methanesulfonyl chloride (0.35 ml), and the mixture was stirred at room temperature for 1 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated to give a yellow oil.

To a solution of pentane-1-sulfonamide (890 mg) in N,N-dimethylformamide (10 ml) was added sodium hydride (60% in oil, 233 mg) under ice-cooling, and the mixture was stirred for 30 min. To the reaction mixture was added dropwise a solution of the orange oil obtained earlier in N,N-dimethylformamide (10 ml), and the mixture was stirred at 50° C. for 60 hr. After cooling to room temperature, a saturated aqueous ammonium chloride solution was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:9-35:65, v/v) to give a white solid. Recrystallization from ethyl acetate-hexane gave N-{3-[2-{[3-chloro-5-(trifluoromethyl)pyridin-2-yl]oxy}-4-(2-methoxyethoxy)phenyl]-2-methylpropyl}pentane-1-sulfonamide (355 mg, yield: 22%) as white crystals. melting point 80-82° C.

### Example 42

To a solution of 3-{2-[(2,4-dichlorobenzyl)oxy]-4-isopropoxyphenyl}propionic acid (1.01 g) in tetrahydrofuran (30 ml) was added N,N′-carbonyldiimidazole (651 mg), and the mixture was heated under reflux for 1 hr. After cooling to room temperature, pentane-1-sulfonamide (439 mg) and 1,8-diazabicyclo[5.4.0]undec-7-ene (0.60 ml) were added to the reaction mixture, and the mixture was stirred overnight. The reaction mixture was concentrated and the concentrate was dissolved in ethyl acetate. The organic layer was washed with saturated aqueous ammonium chloride solution and saturated brines dried (MgSO₄), filtrated and concentrated. The obtained residue was subjected to silica gel column chromatography, and eluted with ethyl acetate-hexane (1:19-1:1, v/v) to give a white solid. Recrystallization from ethyl acetate-hexane gave 3-{2-[(2,4-dichlorobenzyl)oxy]-4-isopropoxyphenyl}-N-(pentylsulfonyl)propanamide (713 mg, yield: 52%) as white crystals. melting point 120.0-120.5° C.

### Example 43

To a solution of 3-[2-{[3-chloro-5-(trifluoromethyl)pyridin-2-yl]oxy}-4-(2-methoxyethoxy)phenyl]propionic acid (4776 mg) in tetrahydrofuran (10 ml) was added N,N′-carbonyldiimidazole (308 mg), and the mixture was heated under reflux for 1 hr. After cooling to room temperature, 3-methylbutane-1-sulfonamide (259 mg) and 1,8-diazabicyclo[5.4.0]undec-7-ene (0.25 ml) were added to the reaction mixture, and the mixture was stirred overnight. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v), then subjected to reversed-phase high performance liquid chromatography, and eluted with acetonitrile-water (2:3 to acetonitrile alone, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]-N-[(3-methylbutyl)sulfonyl]propanamide (137 mg, yield: 22%) as white crystals. melting point 130-132° C.

### Example 44

To a solution of ethyl 3-{2-{[3-chloro-5-(trifluoromethyl)pyridin-2-yl]oxy}-4-[2-(diethylamino)-2-oxoethoxy]phenyl}propionate (3.31 g) in tetrahydrofuran (15 ml) and ethanol (15 ml) was added a 1N aqueous sodium hydroxide solution (15.0 ml), and the mixture was stirred at 50° C. for 1 hr. After cooling to room temperature, 1N hydrochloric acid (15.0 ml) was added, and the mixture was diluted with toluene and concentrated. The residue was dissolved in ethyl acetate. The organic layer was washed twice with saturated brine, dried (MgSO₄), filtrated and concentrated to give 3-{2-{[3-chloro-5-(trifluoromethyl)pyridin-2-yl]oxy}-4-[2-(diethylamino)-2-oxoethoxy]phenyl}propionic acid (2.91 g, yield: 93%) as a pale-yellow solid.

To a solution of 3-{2-{[3-chloro-5-(trifluoromethyl)pyridin-2-yl]oxy}-4-[2-(diethylamino)-2-oxoethoxy]phenyl}propionic acid (2.23 g) in tetrahydrofuran (30 ml) was added N,N′-carbonyldiimidazole (1.17 g), and the mixture was heated under reflux for 1 hr. After cooling to room temperature, pentane-1-sulfonamide (1.07 g) and 1,8-diazabicyclo[5.4.0]undec-7-ene (1.00 ml) were added to the reaction mixture, and the mixture was stirred overnight. 1N Hydrochloric acid was added to the reaction mixture, and the mixture was diluted with ethyl acetate. The organic layer was washed with saturated aqueous sodium hydrogencarbonate solution and saturated brine, dried (MgSO₄), filtrated and concentrated. The obtained residue was subjected to silica gel column chromatography, and eluted with ethyl acetate-hexane (15:85-7:3, v/v) to give a white solid. Recrystallization from ethyl acetate-diisopropyl ether gave 3-{2-{[3-chloro-5-(trifluoromethyl)pyridin-2-yl]oxy}-4-[2-(diethylamino)-2-oxoethoxy]phenyl}-N-(pentylsulfonyl)propanamide (1.96 g, yield: 69%) as a white powder. melting point 111-113° C.

### Example 45

To a solution of a crude product (0.67 g) of 3-{2-{[3-chloro-5-(trifluoromethyl)pyridin-2-yl]oxy}-4-[2-(diethylamino)-2-oxoethoxy]phenyl}propionic acid in tetrahydrofuran (30 ml) was added N,N′-carbonyldiimidazole (363 mg), and the mixture was heated under reflux for 1 hr. After cooling to room temperature, 3-methylbutane-1-sulfonamide (343 mg) and 1,8-diazabicyclo[5.4.0]undec-7-ene (0.50 ml) were added to the reaction mixture, and the mixture was stirred overnight. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (15:85-7:3, v/v) to give a white solid. Recrystallization from ethyl acetate-diisopropyl ether gave 3-{2-{[3-chloro-5-(trifluoromethyl)pyridin-2-yl]oxy}-4-[2-(diethylamino)-2-oxoethoxy]phenyl}-N-[(3-methylbutyl)sulfonyl]propanamide (285 mg, yield: 33%) as white feather crystals. melting point 123-125° C.

### Example 46

To a solution of 3-[2-{[3-chloro-5-(trifluoromethyl)pyridin-2-yl]oxy}-4-(2-methoxyethoxy)phenyl]propionic acid (945 mg) in tetrahydrofuran (40 ml) was added N,N′-carbonyldiimidazole (622 mg), and the mixture was heated under reflux for 1 hr. After cooling to room temperature, 3-methoxypropane-1-sulfonamide (415 mg) and 1,8-diazabicyclo[5.4.0]undec-7-ene (0.60 ml) were added to the reaction mixture, and the mixture was stirred overnight. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1′, v/v), and then subjected to reversed-phase high performance liquid chromatography, and eluted with acetonitrile-water (2:3 to acetonitrile alone, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]-N-[(3-methoxypropyl)sulfonyl]propanamide (622 mg, yield: 50%) as white feather crystals. melting point 112-113° C.

### Example 47

To a solution of 3-{2-{[3-chloro-5-(trifluoromethyl)pyridin-2-yl]oxy}-4-[2-(2-oxopyrrolidin-1-yl)ethoxy]phenyl}propanoic acid (0.70 g) in tetrahydrofuran (15 ml) was added N,N′-carbonyldiimidazole (392 mg), and the mixture was heated under reflux for 1 hr. After cooling to room temperature, pentane-1-sulfonamide (342 mg) and 1,8-diazabicyclo[5.4.0]undec-7-ene (0.50 ml) were added to the reaction mixture, and the mixture was stirred overnight. A saturated aqueous ammonium chloride solution was added to the reaction mixture, and the mixture was diluted with ethyl acetate. The organic layer was washed with water, 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (15:85 to ethyl acetate alone, v/v) to give a white solid. Recrystallization from ethyl acetate-diisopropyl ether gave 3-{2-{[3-chloro-5-(trifluoromethyl)pyridin-2-yl]oxy}-4-[2-(2-oxopyrrolidin-1-yl)ethoxy]phenyl}-N-(pentylsulfonyl)propanamide (668 mg, yield: 74%) as a white powder. melting point 123-126° C.

### Example 48

To a solution of 3-[2-{[3-chloro-5-(trifluoromethyl)pyridin-2-yl]oxy}-4-(2-methoxyethoxy)phenyl]propan-1-ol (0.30 g) in N,N-dimethylformamide (7 ml) was added N,N′-carbonyldiimidazole (0.18 g), and the mixture was stirred at 60° C. for 1.5 hr. Morpholine (193 μl) was added to the reaction mixture, and the mixture was stirred overnight. After cooling to room temperature, water was added to the reaction mixture, and the mixture was extracted with ethyl acetate. The organic layer was washed with saturated brine, dried (MgSO₄), filtrated and concentrated. The obtained residue was subjected to silica gel column chromatography, and eluted with ethyl acetate-hexane (5:95-3:7, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]propyl morpholine-4-carboxylate (372 mg, yield: 98%) as a white powder. melting point 82.3-82.4° C.

### Example 49

To a solution of 3-[2-{[3-chloro-5-(trifluoromethyl)pyridin-2-yl]oxy}-4-(2-methoxyethoxy)phenyl]propan-1-ol (0.30 g) in N,N-dimethylformamide (7 ml) was added N,N′-carbonyldiimidazole (0.18 g), and the mixture was stirred at 60° C. for 1 hr. 1-Butylamine (0.22 ml) was added to the reaction mixture, and the mixture was stirred for 6 hr. After cooling to room temperature, water was added to the reaction mixture, and the mixture was extracted with ethyl acetate. The organic layer was washed with saturated brine, dried (MgSO₄), filtrated and concentrated. The obtained residue was subjected to silica gel column chromatography, and eluted with ethyl acetate-hexane (1:9-3:7, v/v) to give a white solid. Recrystallization from diisopropyl ether-hexane gave 3-[2-{[3-chloro-5-(trifluoromethyl)pyridin-2-yl]oxy}-4-(2-methoxyethoxy)phenyl]propyl butylcarbamate (297 mg, yield: 80%) as a white powder. melting point 91.7-91.8° C.

### Example 50

To a solution of 3-[2-{[3-chloro-5-(trifluoromethyl)pyridin-2-yl]oxy}-4-(2-methoxyethoxy)phenyl]propan-1-ol (0.30 g) in N,N-dimethylformamide (7 ml) was added N,N′-carbonyldiimidazole (0.18 g), and the mixture was stirred at 60° C. for 1 hr. N-Methyl-1-butylamine (0.26 ml) was added to the reaction mixture, and the mixture was stirred for 6.5 hr. N-Methyl-1-butylamine (0.26 ml) was further added, and the mixture was stirred overnight. After cooling to room temperature, water was added to the reaction mixture, and the mixture was extracted with ethyl acetate. The organic layer was washed with saturated brine, dried (MgSO₄), filtrated and concentrated. The obtained residue was subjected to silica gel column chromatography, and eluted with ethyl acetate-hexane (1:9-3:7, v/v) to give a white solid. Recrystallization from hexane gave 3-[2-{[3-chloro-5-(trifluoromethyl)pyridin-2-yl]oxy}-4-(2-methoxyethoxy)phenyl]propyl butyl(methyl)carbamate (271 mg, yield: 73%) as a white powder. melting point 54.0-55.2° C.

### Example 51

To absolution of 3-[2-{[3-chloro-5-(trifluoromethyl)pyridin-2-yl]oxy}-4-(2-methoxyethoxy)phenyl]propan-1-ol (0.30 g) in N,N-dimethylformamide (7 ml) was added N,N′-carbonyldiimidazole (0.18 g), and the mixture was stirred at 60° C. for 1.5 hr. Cyclopropylmethylamine (385 μl) was added to the reaction mixture, and the mixture was stirred overnight. After cooling to room temperature, water was added to the reaction mixture, and the mixture was extracted with ethyl acetate. The organic layer was washed with saturated brine, dried (MgSO₄), filtrated and concentrated. The obtained residue was subjected to silica gel column chromatography, and eluted with ethyl acetate-hexane (1:9′-3:7, v/v) to give a white solid. Recrystallization from diisopropyl ether-hexane gave 3-[2-{[3-chloro-5-(trifluoromethyl)pyridin-2-yl]oxy}-4-(2-methoxyethoxy)phenyl]propyl (cyclopropylmethyl)carbamate (272 mg, yield: 73%) as a white powder. melting point 87.8-87.9° C.

### Example 52

To a solution of 3-(2-{[3-chloro-5-(trifluoromethyl)pyridin-2-yl]oxy}-4-hydroxyphenyl)propyl (cyclopropylmethyl)carbamate (342 mg) in N,N-dimethylformamide (5 ml) were added potassium carbonate (225 mg) and 2-chloro-N,N-diethylacetamide (182 mg), and the mixture was stirred at 50° C. for 1 hr. After cooling to room temperature, 1N hydrochloric acid was added to the reaction mixture, and the mixture was extracted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-3:2, v/v) to give a white solid. Recrystallization from ethyl acetate-hexane gave 3-{2-{[3-chloro-5-(trifluoromethyl)pyridin-2-yl]oxy}-4-[2-(diethylamino)-2-oxoethoxy]phenyl}propyl (cyclopropylmethyl)carbamate (346 g, yield: 81%) as white crystals. melting point 94.0-94.5° C.

### Example 53

To a solution of 3-(2-{[3-chloro-5-(trifluoromethyl)pyridin-2-yl]oxy}-4-hydroxyphenyl)propyl (cyclopropylmethyl)carbamate (381 mg) in N,N-dimethylformamide (5 ml) were added potassium carbonate (252 mg) and ethyl bromoacetate (0.20 ml), and the mixture was stirred at 50° C. for 30 min. After cooling to room temperature, 1N hydrochloric acid was added to the reaction mixture, and the mixture was extracted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give ethyl {3-{[3-chloro-5-(trifluoromethyl)-pyridin-2-yl]oxy}-4-[3-({[(cyclopropylmethyl)amino]carbonyl}-oxy)propyl]phenoxy}acetate (383 mg, yield: 84%) as a white solid. Recrystallization from ethyl acetate-hexane gave white feather crystals. melting point 112.8-113.0° C.

### Example 54

To a solution of ethyl {3-{[3-chloro-5-(trifluoromethyl)pyridin-2-yl]oxy}-4-[3-({[(cyclopropylmethyl)amino]carbonyl}oxy)propyl]-phenoxy}acetate (301 mg) in tetrahydrofuran (5 ml) and ethanol (5 ml) was added a 1N aqueous sodium hydroxide solution (2.0 ml), and the mixture was stirred overnight at room temperature. 1N Hydrochloric acid (2.0 ml) was added to the reaction mixture, and the mixture was diluted with toluene and concentrated. The residue was dissolved in ethyl acetate. The organic layer was washed with water and saturated brine, dried (MgSO₄), filtrated and concentrated. The residue was recrystallized from ethyl acetate-hexane to give {3-{[3-chloro-5-(trifluoromethyl)pyridin-2-yl]oxy}-4-[3-({[(cyclopropylmethyl)amino]carbonyl}oxy)propyl]phenoxy}acetic acid (284 mg, yield: quant.) as white feather crystals.

¹H-NMR (300 MHz, CDCl₃) δ:0.14-0.21 (2H, m), 0.42-0.53 (2H, m), 0.85-1.05 (1H, m), 1.74-1.97 (2H, m), 2.48-2.58 (2H, m), 2.96-3.06 (2H, m), 4.02 (2H, t, J=6.3 Hz), 4.64 (2H, s), 4.72 (1H, s), 6.71 (1H, d, J=2.6 Hz), 6.82 (1H, dd, J=8.5, 2.4 Hz), 7.24 (1H, d, J=8.7 Hz), 7.99 (1H, d, J=2.1 Hz), 8.26 (1H, d, J=0.9 Hz).

### Example 55

To a solution of 3-[2-{[3-chloro-5-(trifluoromethyl)pyridin-2-yl]oxy}-4-(2-methoxyethoxy)phenyl]propan-1-ol (400 mg) in N,N-dimethylformamide (5 ml) was added N,N′-carbonyldiimidazole (222 mg), and the mixture was stirred at 40° C. for 1 hr. 3-Methoxypropylamine (0.55 ml) was added to the reaction mixture, and the mixture was stirred for 1 hr. After cooling to room temperature, 1N hydrochloric acid was added to the reaction mixture, and the mixture was extracted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-3:2, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]propyl (3-methoxypropyl)carbamate (276 mg, yield: 54%) as white feather crystals. melting point 83.5-85.0° C.

### Example 56

To a solution of 3-[2-{[3-chloro-5-(trifluoromethyl)pyridin-2-yl]oxy}-4-(2-methoxyethoxy)phenyl]propan-1-ol (405 mg) in N,N-dimethylformamide (5 ml) was added N,N′-carbonyldiimidazole (224 mg), and the mixture was stirred at 40° C. for 1 hr. 2-Ethoxyethylamine (0.55 ml) was added to the reaction mixture, and the mixture was stirred for 1 hr. After cooling to room temperature, 1N hydrochloric acid was added to the reaction mixture, and the mixture was extracted with ethyl acetate. The organic layer was washed with water and saturated brine, dried (MgSO₄), filtrated and concentrated. The obtained residue was subjected to silica gel column chromatography, and eluted with ethyl acetate-hexane (1:19-2:3, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]propyl (2-ethoxyethyl)carbamate (250 mg, yield: 48%) as a white powder. melting point 66-68° C.

### Example 57

To a solution of (2E)-3-[2-{[3-chloro-5-(trifluoromethyl)pyridin-2-yl]oxy}-4-isopropoxyphenyl]acrylic acid (598.8 mg) in tetrahydrofuran (10 ml) was added N,N′-carbonyldiimidazole (368 mg), and the mixture was heated under reflux for 1 hr. After cooling to room temperature, 3-methoxypropane-1-sulfonamide (355 mg) and 1,8-diazabicyclo[5.4.0]undec-7-ene (0.40 ml) were added to the reaction mixture, and the mixture was stirred for 3 hr. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:5-9:1, v/v) to give a white solid. Recrystallization from ethyl acetate-hexane gave (2E)-3-[2-{[3-chloro-5-(trifluoromethyl)pyridin-2-yl]oxy}-4-isopropoxyphenyl]-N-[(3-methoxypropyl)sulfonyl]acrylamide (296 mg, yield: 37%) as a white powder. melting point 129.5-130.0° C.

### Example 58

To a solution of [2-{[3-chloro-5-(trifluoromethyl)pyridin-2-yl]oxy}-4-(methoxyethoxy)phenyl]methanol (473 mg) in N,N-dimethylformamide (5 ml) was added N,N′-carbonyldiimidazole (295.3 mg), and the mixture was stirred at room temperature for 1 hr and at 40° C. for 30 min. Then, pentane-1-sulfonamide (289 mg), 1,8-diazabicyclo[5.4.0]undec-7-ene (0.30 ml) and 4-dimethylaminopyridine (215 mg) were added, and the mixture was subsequently stirred overnight. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:7-1:4, v/v) to give a white solid. Recrystallization from ethyl acetate-hexane gave 2-{[3-chloro-5-(trifluoromethyl)pyridin-2-yl]oxy}-4-(2-methoxyethoxy)benzyl (pentylsulfonyl)carbamate (373 mg, yield: 54%) as white crystals. melting point 130-132° C.

### Example 59

To a solution of triphosgene (445 mg) in toluene (10 ml) was added a solution of 3-methylbutane-1-sulfonamide (659 mg) in toluene (5 ml) and pyridine (0.40 ml) under ice-cooling, and the mixture was stirred at room temperature for 1 hr. Then, [2-{[3-chloro-5-(trifluoromethyl)pyridin-2-yl]oxy}-4-(2-methoxyethoxy)phenyl]methanol (1.12 g), N,N-diisopropylethylamine (0.75 ml), 4-dimethylaminopyridine (574 mg) and tetrahydrofuran (15 ml) were added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/v) to give a white solid. Recrystallization from ethyl acetate-hexane gave 2-{[3-chloro-5-(trifluoromethyl)pyridin-2-yl]oxy}-4-(2-methoxyethoxy)benzyl [(3-methylbutyl)sulfonyl]carbamate (19 mg, yield: 2%) as white feather crystals. melting point 122-124° C.

### Example 60

To a solution of [2-{[3-chloro-5-(trifluoromethyl)pyridin-2-yl]oxy}-4-(2-methoxyethoxy)phenyl]methanol (501 mg) in toluene (20 ml) and tetrahydrofuran (5 ml) was added chlorosulfonylisocyanate (120 μl) with stirring under ice-cooling, and 30 min later, pyridine (0.25 ml) was added. After 1 hr, isobutylamine (0.75 ml) was added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, eluted with ethyl acetate-hexane (1:9-3:2, v/v), and recrystallized from ethyl acetate-hexane to give 2-{[3-chloro-5-(trifluoromethyl)pyridin-2-yl]oxy}-4-(2-methoxyethoxy)benzyl [(isobutylamino)sulfonyl]carbamate (13 mg, yield: 2%) as white crystals. melting point 160-161° C.

### Example 61

To a solution of [2-{[3-chloro-5-(trifluoromethyl)pyridin-2-yl]oxy}-4-(2-methoxyethoxy)phenyl]methanol (507 mg) in toluene (20 ml) and tetrahydrofuran (5 ml) was added chlorosulfonylisocyanate (120 μl) with stirring under ice-cooling, and 30 min later, pyridine (0.25 ml) was added. After 1 hr, 1-pentylamine (0.75 ml) was added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-3:2, v/v), and recrystallized from ethyl acetate-hexane to give 2-{[3-chloro-5-(trifluoromethyl)pyridin-2-yl]oxy}-4-(2-methoxyethoxy)benzyl [(pentylamino)sulfonyl]carbamate (33 mg, yield: 4%) as white crystals. melting point 153-155° C.

### Example 62

To a solution of (4-butoxy-2-{[3-chloro-5-(trifluoromethyl)pyridin-2-yl]oxy}phenyl)methanol (1.04 g) in N,N-dimethylformamide (10 ml) was added N,N′-carbonyldiimidazole (592 mg), and the mixture was stirred at 40° C. for 1 hr. Then, pentane-1-sulfonamide (584 mg) and 1,8-diazabicyclo[5.4.0]undec-7-ene (0.60 ml) were added, and the mixture was subsequently stirred for 2 hr. After cooling to room temperature, 1N hydrochloric acid was added to the reaction mixture, and the mixture was diluted with ethyl acetate. The organic layer was washed with water, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give a white solid. Recrystallization from ethyl acetate-hexane gave 4-butoxy-2-{[3-chloro-5-(trifluoromethyl)pyridin-2-yl]oxy}benzyl (pentylsulfonyl)carbamate (379 mg, yield: 25%) as a white powder. melting point 124.5-125.5° C.

### Example 63

To a solution of (2-{[3-chloro-5-(trifluoromethyl)pyridin-2-yl]oxy}-4-isopropoxyphenyl)methanol (1.00 g) in N,N-dimethylformamide (10 ml) was added N,N′-carbonyldiimidazole (621 mg), and the mixture was stirred at 40° C. for 1 hr. Then, pentane-1-sulfonamide (594 mg) and 1,8-diazabicyclo[5.4.0]undec-7-ene (0.6b ml) were added, and the mixture was subsequently stirred for 2 hr. After cooling to room temperature, 1N hydrochloric acid was added to the reaction mixture, and the mixture was diluted with ethyl acetate. The organic layer was washed with water, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-45:55, v/v) to give a white solid. Recrystallization from ethyl acetate-hexane gave 2-{[3-chloro-5-(trifluoromethyl)pyridin-2-yl]oxy}-4-isopropoxybenzyl (pentylsulfonyl)carbamate (500 mg, yield: 34%) as a white powder. melting point 70-73° C.

### Example 64

To a solution of 3-{[3-chloro-5-(trifluoromethyl)pyridin-2-yl]oxy}-4-(hydroxymethyl)phenyl propane-1-sulfonate (1.02 g) in N,N-dimethylformamide (10 ml) was added N,N′-carbonyldiimidazole (613 mg), and the mixture was stirred at 40° C. for 1 hr. Then, pentane-1-sulfonamide (569 mg) and 1,8-diazabicyclo[5.4.0]undec-7-ene (0.60 ml) were added, and the mixture was subsequently stirred for 2 hr. After cooling to room temperature, 1N hydrochloric acid was added to the reaction mixture, and the mixture was diluted with ethyl acetate. The organic layer was washed with water, 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give a white solid. Recrystallization from ethyl acetate-hexane gave 3-{[3-chloro-5-(trifluoromethyl)pyridin-2-yl]oxy}-4-[({[(pentylsulfonyl)amino]carbonyl}oxy)methyl]phenyl propane-1-sulfonate (127 mg, yield: 13%) as white feather crystals. melting point 86-89° C.

### Example 65

To a solution of [2-{[3-chloro-5-(trifluoromethyl)pyridin-2-yl]oxy}-4-(2-methoxyethoxy)phenyl]methanol (1.08 g) in tetrahydrofuran (15 ml) were added N,N-diisopropylethylamine (0.50 ml), 4-chlorobenzenesulfonylisocyanate (0.44 ml) and 4-dimethylaminopyridine (355 mg), and the mixture was stirred at room temperature for 30 min. 4-Chlorobenzenesulfonylisocyanate (0.44 ml) was further added, and the mixture was stirred for 1 hr. A saturated aqueous ammonium chloride solution was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/v) to give a colorless solid. Recrystallization from ethyl acetate-hexane gave 2-{[3-chloro-5-(trifluoromethyl)pyridin-2-yl]oxy}-4-(2-methoxyethoxy)benzyl [(4-chlorophenyl)sulfonyl]carbamate (264 mg, yield: 16%) as a white powder. melting point 112-115° C.

### Example 66

To a solution of [2-{[3-chloro-5-(trifluoromethyl)pyridin-2-yl]oxy}-4-(2-methoxyethoxy)phenyl]methanol (520 mg) in toluene (20 ml) was added chlorosulfonylisocyanate (0.15 ml) with stirring under ice-cooling, and 30 min later, pyridine (0.30 ml) was added. After 1 hr, 1-butylamine (0.80 ml) was added, and the mixture was stirred overnight at room temperature. 1N Hydrochloric acid was added to the reaction mixture, and the mixture was diluted with ethyl acetate. The organic layer was washed with water, a saturated aqueous sodium hydrogencarbonate solution and saturated brine, dried (MgSO₄), filtrated and concentrated to give a white solid. Recrystallization from ethyl acetate-diisopropyl ether gave 2-{[3-chloro-5-(trifluoromethyl)pyridin-2-yl]oxy}-4-(2-methoxyethoxy)benzyl [(butylamino)sulfonyl]carbamate (542 mg, yield: 71%) as a white powder. melting point 164-165° C.

### Example 67

To a solution of [2-{[3-chloro-5-(trifluoromethyl)pyridin-2-yl]oxy}-4-(2-methoxyethoxy)phenyl]methanol (521 mg) in toluene (20 ml) was added chlorosulfonylisocyanate (0.15 ml) with stirring under ice-cooling, and 30 min later, pyridine (0.30 ml) was added, and after 1 hr, cyplopropylmethylamine (750 mg) was added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated to give a white solid. The obtained solid was washed with diisopropyl ether, and recrystallized from ethyl acetate-diisopropyl ether to give 2-{[3-chloro-5-(trifluoromethyl)pyridin-2-yl]oxy}-4-(2-methoxyethoxy)benzyl {[(cyclopropylmethyl)amino]sulfonyl}carbamate (481 mg, yield: 63%) as a white powder. melting point 150.0-150.5° C.

### Example 68

To a solution of [2-{[3-chloro-5-(trifluoromethyl)pyridin-2-yl]oxy}-4-(2-methoxyethoxy)phenyl]methanol (513 mg) in toluene (20 ml) was added chlorosulfonylisocyanate (0.15 ml) with stirring under ice-cooling, and 30 min later, pyridine (0.30 ml) was added. After 1 hr, p-chloroaniline (659 mg) was added, and the mixture was stirred at room temperature for 4 hr. 1N Hydrochloric acid was added to the reaction mixture, and the mixture was diluted with ethyl acetate. The organic layer was washed with water, a saturated aqueous sodium hydrogencarbonate solution and saturated brine, dried (MgSO₄), filtrated and concentrated to give a white solid. The obtained solid was washed with diisopropyl ether, and recrystallized from ethyl acetate-diisopropyl ether to give 2-{[3-chloro-5-(trifluoromethyl)pyridin-2-yl]oxy}-4-(2-methoxyethoxy)benzyl {[(4-chlorophenyl)amino]sulfonyl}carbamate (32.1 mg, yield: 4%) as a white powder. melting point 159° C. (dec.).

### Example 69

To a solution of [2-{[3-chloro-5-(trifluoromethyl)pyridin-2-yl]oxy}-4-(2-methoxyethoxy)phenyl]methanol (541 mg) in toluene (20 ml) was added chlorosulfonylisocyanate (0.15 ml) with stirring under ice-cooling, and 30 min later, pyridine (0.30 ml) was added. After 1 hr, morpholine (0.80 ml) was added, and the mixture was stirred at room temperature for 4 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/v) to give 2-{[3-chloro-5-(trifluoromethyl)pyridin-2-yl]oxy}-4-(2-methoxyethoxy)benzyl (morpholin-4-ylsulfonyl)carbamate (65 mg, 8%) as a colorless oil.

¹H-NMR (300 MHz, CDCl₃) δ:3.24-3.34 (4H, m), 3.44 (3H, s), 3.59-3.79 (6H, m), 4.07-4.18 (2. H, m), 5.10 (2H, s), 6.71 (1H, s), 6.75 (1H, d, J=2.4 Hz), 6.88 (1H, dd, J=8.6, 2.5 Hz), 7.41 (1H, d, J=8.5 Hz), 8.01 (1H, d, J=1.9 Hz), 8.27 (1H, dd, J=2.2, 1.0 Hz).

### Example 70

To a solution of [2-{[3-chloro-5-(trifluoromethyl)pyridin-2-yl]oxy}-4-(2-methoxyethoxy)phenyl]methanol (506 mg) in toluene (20 ml) was added chlorosulfonylisocyanate (0.15 ml) with stirring under ice-cooling, and 30 min later, pyridine (0.30 ml) was added. After 1 hr, 1-hexylamine (0.80 ml) was added, and the mixture was stirred overnight at room temperature. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, eluted with ethyl acetate-hexane (1:9-2:3, v/v) and recrystallized from ethyl acetate-diisopropyl ether to give 2-{[3-chloro-5-(trifluoromethyl)pyridin-2-yl]oxy}-4-(2-methoxyethoxy)benzyl [(hexylamino)sulfonyl]carbamate (71 mg, yield: 9%) as white crystals.

¹H-NMR (300 MHz, CDCl₃) δ:0.79-0.97 (3H, m), 1.17-1.38 (6H, m), 1.40-1.55 (2H, m), 2.94 (2H, q, J=7.0 Hz), 3.44 (3H, s), 3.69-3.79 (2H, m), 4.07-4.15 (2H, m), 5.14 (2H, s), 5.19 (1H, t, J=6.2 Hz), 6.73 (1H, d, J=2.4 Hz), 6.87 (1H, dd, J=8.5, 2.4 Hz), 7.15 (1H, s), 7.42 (1H, d, J=8.5 Hz), 8.02 (1H, d, J=1.9 Hz), 8.26 (1H, dd, J=2.1, 0.9 Hz).

### Example 71

To a solution of [2-{[3-chloro-5-(trifluoromethyl)pyridin-2-yl]oxy}-4-(2-methoxyethoxy)phenyl]methanol (500 mg) in N,N-dimethylformamide (5 ml) was added N,N′-carbonyldiimidazole (337 mg), and the mixture was stirred at 40° C. for 1 hr. Then, N-hexyl-N-methylsulfamide (389 mg) and 1,8-diazabicyclo[5.4.0]undec-7-ene (0.30 ml) were added, and the mixture was stirred at room temperature for 5 hr. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/v) to give 2-{[3-chloro-5-(trifluoromethyl)pyridin-2-yl]oxy}-4-(2-methoxyethoxy)benzyl {[hexyl(methyl)amino]sulfonyl}carbamate (85 mg, yield: 11%) as a colorless oil.

¹H-NMR (300 MHz, CDCl₃) δ:0.76-0.97 (3H, m), 1.14-1.36 (4H, m), 1.41-1.64 (4H, m), 2.87 (3H, s), 3.09-3.23 (2H, m), 3.44 (3H, s), 3.67-3.85 (2H, m), 4.07-4.18 (2H, m), 5.08 (2H, s), 6.74 (1H, d, J=2.4 Hz), 6.87 (1H, dd, J=8.6, 2.5 Hz), 7.04 (1H, s), 7.41 (1H, d, J=8.5 Hz), 8.00 (1H, d, J=1.9 Hz), 8.26 (1H, dd, J=2.1, 0.9 Hz).

### Example 72

To a solution of 3-[2-{[3-chloro-5-(trifluoromethyl)pyridin-2-yl]oxy}-4-(2-methoxyethoxy)phenyl]propionic acid (511 mg) in tetrahydrofuran (6 ml) was added N,N′-carbonyldiimidazole (314 mg), and the mixture was heated under reflux for 1 hr. After cooling to room temperature, N-hexyl-N-methylsulfamide (250 mg) and 1,8-diazabicyclo[5.4.0]undec-7-ene (0.30 ml) were added to the reaction mixture, and the mixture was stirred for 10 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give 3-[2-{[3-chloro-5-(trifluoromethyl)pyridin-2-yl]oxy}-4-(2-methoxyethoxy)phenyl]-N-{[hexyl(methyl)amino]sulfonyl}propanamide (59 mg, yield: 8%) as a colorless oil.

¹H-NMR (300 MHz, CDCl₃) δ:0.83-0.93 (3H, m), 1.19-1.37 (6H, m); 1.44-1.57 (2H, m), 2.52 (2H, t, J=7.3 Hz), 2.76 (3H, s), 2.89 (2H, t, J=7.3 Hz), 3.01-3.12 (2H, m), 3.42 (3H, s), 3.68-3.74 (2H, m), 3.97-4.11 (2H, m), 6.56 (1H, d, J=2.4 Hz), 6.79 (1H, dd, J=8.5, 2.6 Hz), 7.19 (1H, d, J=8.7 Hz), 8.05 (1H, d, J=1.9 Hz), 8.35 (1H, dd, J=2.1, 0.8 Hz), 8.67 (1H, s)

### Example 73

To a solution of 3-[2-{[3-chloro-5-(trifluoromethyl)pyridin-2-yl]oxy}-4-(2-methoxyethoxy)phenyl]propionic acid (508 mg) in tetrahydrofuran (5 ml) was added N,N′-carbonyldiimidazole (296.1 mg), and the mixture was heated under reflux for 1 hr. After cooling to room temperature, sulfamide (221 mg) and 1,8-diazabicyclo[5.4.0]undec-7-ene (0.30 ml) were added to the reaction mixture, and the mixture was stirred overnight. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was recrystallized from tetrahydrofuran-diisopropyl ether to give N-(aminosulfonyl)-3-[2-{[3-chloro-5-(trifluoromethyl)pyridin-2-yl]oxy}-4-(2-methoxyethoxy)phenyl]propanamide (489 mg, yield: 81%) as a white powder. melting point 158-159° C.

### Example 74

To a solution of 4-[2-{[3-chloro-5-(trifluoromethyl)pyridin-2-yl]oxy}-4-(2-methoxyethoxy)phenyl]butanoic acid (550.1 mg) in tetrahydrofuran (5 ml) was added N,N′-carbonyldiimidazole (321 mg), and the mixture was heated under reflux for 1 hr. After cooling to room temperature, pentane-1-sulfonamide (261 mg) and 1,8-diazabicyclo[5.4.0]undec-7-ene (0.30 ml) were added to the reaction mixture, and the mixture was stirred overnight. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/v) to give a white solid. Recrystallization from ethyl acetate-hexane gave 4-[2-{[3-chloro-5-(trifluoromethyl)pyridin-2-yl]oxy}-4-(2-ethoxyethoxy)phenyl]-N-(pentylsulfonyl)butanamide (151 mg, yield: 21%) as white feather crystals. melting point 96-97° C.

### Example 75

To a solution of [2-{[3-chloro-5-(trifluoromethyl)pyridin-2-yl]oxy}-4-(2-methoxyethoxy)phenyl]methanol (500 mg) in dichloromethane (15 ml) was added chlorosulfonylisocyanate (0.15 ml) with stirring under ice-cooling, and 30 min later, pyridine (0.30 ml) was added. After 1 hr, 3-methoxypropylamine (0.80 ml) was added, and the mixture was stirred overnight at room temperature. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/v) to give a white solid. Recrystallization from ethyl acetate-hexane gave 2-{[3-chloro-5-(trifluoromethyl)pyridin-2-yl]oxy}-4-(2-methoxyethoxy)benzyl {[(3-methoxypropyl)amino]sulfonyl}carbamate (458 mg, yield: 61%) as white feather crystals. melting point 149.0-149.5° C.

### Example 76

To a solution of [2-{[3-chloro-5-(trifluoromethyl)pyridin-2-yl]oxy}-4-(2-methoxyethoxy)phenyl]methanol (455 mg) in dichloromethane (15 ml) was added chlorosulfonylisocyanate (0.15 ml) with stirring under ice-cooling, and 30 min later, pyridine (0.30 ml) was added. After 1 hr. 2-ethoxyethylamine (0.80 ml) was added, and the mixture was stirred overnight at room temperature. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/v) to give a white solid. Recrystallization from ethyl acetate-hexane gave 2-{[3-chloro-5-(trifluoromethyl)pyridin-2-yl]oxy}-4-(2-methoxyethoxy)benzyl {[(2-ethoxyethyl)amino]sulfonyl}carbamate (301 mg, yield: 44%) as white feather crystals. melting point 142.5-143.0° C.

### Example 77

To a solution of 3-[2-{[3-chloro-5-(trifluoromethyl)pyridin-2-yl]oxy}-4-(2-methoxyethoxy)phenyl]butanoic acid (518.8 mg) in tetrahydrofuran (10 ml) was added N,N′-carbonyldiimidazole (309 mg), and the mixture was heated under reflux for 1 hr. After cooling to room temperature, pentane-1-sulfonamide (218 mg) and 1,8-diazabicyclo[5.4.0]undec-7-ene (0.30 ml) were added to the reaction mixture, and the mixture was stirred overnight. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-2:3, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]-N-(pentylsulfonyl)butanamide (251 mg, yield: 37%) as white crystals. melting point 136.9-137.1° C.

### Example 78

To a solution of 3-[2-{[3-chloro-5-(trifluoromethyl)pyridin-2-yl]oxy}-4-(2-methoxyethoxy)phenyl]butanoic acid (511 mg) in tetrahydrofuran (10 ml) was added N,N′-carbonyldiimidazole (290 mg), and the mixture was heated under reflux for 1 hr. After cooling to room temperature, 3-methoxypropane-1-sulfonamide (230 mg) and 1,8-diazabicyclo[5.4.0]undec-7-ene (0.30 ml) were added to the reaction mixture, and the mixture was stirred overnight. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:3-4:1, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]-N-[(3-methoxypropyl)sulfonyl]butanamide (330 mg, yield: 49%) as white crystals. melting point 107-109° C.

### Example 79

To a solution of 3-[2-{[3-chloro-5-(trifluoromethyl)pyridin-2-yl]oxy}-4-(2-methoxyethoxy)phenyl]propan-1-ol (2.01 g) in tetrahydrofuran (100 ml) were added triphenylphosphine (1.81 g), tert-butyl [(2-nitrophenyl)sulfonyl]carbamate (1.81 g) and diethyl azodicarboxylate 40% toluene solution (3.80 ml) under ice-, cooling, and the mixture was stirred at 0° C. for 10 min and at room temperature for 1 hr. The reaction mixture was concentrated, and the obtained residue was subjected to silica gel column chromatography, and eluted with ethyl acetate-hexane (3:7-7:3, v/v) to give tert-butyl {3-[2-{[3-chloro-5-(trifluoromethyl)pyridin-2-yl]oxy}-4-(2-methoxyethoxy)phenyl]propyl}[(2-nitrophenyl)sulfonyl]carbamate (3.44 g, yield: quant.) as a pale-yellow solid.

¹H-NMR (300 MHz, CDCl₃) δ:1.26-1.32 (9H, m), 1.96-2.08 (2H, m), 2.46-2.59 (2H, m), 3.44 (3H, s), 3.67-3.77 (4H, m), 4.03-4.14 (2H, m), 6.69 (1H, d, J=2.6 Hz), 6.84 (1H, dd, J=8.5, 2.6 Hz), 7.22-7.25 (1H, m), 7.67-7.77 (3H, m), 7.98 (1H, d, J=2.3 Hz), 8.22-8.30 (2H, m).

### Example 80

A mixture of 3-[1-(2,4-difluorobenzyl)-3-isopropoxy-1H-pyrazol-5-yl]propanoic acid (140 mg), N,N′-carbonyldiimidazole (77 mg) and tetrahydrofuran (5.0 ml) was stirred with heating under reflux for 1 hr. After cooling to room temperature, pentane-1-sulfonamide (72 mg) and 1,8-diazabicyclo[5.4.0]undec-7-ene (72 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-1:2, v/v), concentrated, and crystallized from ethyl; acetate-hexane to give 3-[1-(2,4-difluorobenzyl)-3-isopropoxy-1H-pyrazol-5-yl]-N-(pentylsulfonyl)propanamide (50 mg, yield: 25%) as colorless crystals. melting point 107-108° C.

### Example 81

A mixture of 3-[1-(2,4-dichlorobenzyl)-3-isopropoxy-1H-pyrazol-5-yl]propanoic acid (500 mg), N,N′-carbonyldiimidazole (341 mg) and tetrahydrofuran (10 ml) was stirred with heating under reflux for 1 hr. After cooling to room temperature, pentane-1-sulfonamide (254 mg) and 1,8-diazabicyclo[5.4.0]undec-7-ene (320 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-1:2, v/v), concentrated, and crystallized from ethyl acetate-hexane to give 3-[1-(2,4-dichlorobenzyl)-3-isopropoxy-1H-pyrazol-5-yl]-N-(pentylsulfonyl)propanamide (450 mg, yield: 66%) as colorless crystals. melting point 126-129° C.

### Example 82

A mixture of 3-[1-(2,4-dichlorobenzyl)-3-isopropoxy-1H-pyrazol-5-yl]propanoic acid (500 mg), N,N′-carbonyldiimidazole (341 mg) and tetrahydrofuran (10 ml) was stirred with heating under reflux for 1 hr. After cooling to room temperature, benzenesulfonamide (264 mg) and 1,8-diazabicyclo[5.4.0]undec-7-ene (320 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was crystallized from ethyl acetate-hexane to give 3-[1-(2,4-dichlorobenzyl)-3-isopropoxy-1H-pyrazol-5-yl]-N-(phenylsulfonyl)propanamide (170 mg, yield: 24%) as colorless crystals. melting point 184-185° C.

### Example 83

A mixture of 3-[1-(2,4-dichlorobenzyl)-3-isopropoxy-1H-pyrazol-5-yl]propanoic acid (500 mg), N,N′-carbonyldiimidazole (341 mg) and tetrahydrofuran (10 ml) was stirred with heating under reflux for 1 hr. After cooling to room temperature, 4-(trifluoromethyl)benzenesulfonamide (378 mg) and 1,8-diazabicyclo[5.4.0]undec-7-ene (320, mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was crystallized from ethyl acetate-hexane to give 3-[1-(2,4-dichlorobenzyl)-3-isopropoxy-1H-pyrazol-5-yl]-N-{[4-(trifluoromethyl)phenyl]sulfonyl}propanamide (700 mg, yield: 89%) as colorless crystals. melting point 167-169° C.

### Example 84

A mixture of, 3-[1-(2,4-dichlorobenzyl)-3-isopropoxy-1H-pyrazol-5-yl]propanoic acid (500 mg), N,N′-carbonyldiimidazole (341 mg) and tetrahydrofuran (10 ml) was stirred with heating under reflux for 1 hr. After cooling to room temperature, 1-phenylmethanesulfonamide (320 mg) and 1,8-diazabicyclo[5.4.0]undec-7-ene (320 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was crystallized from ethyl acetate-hexane to give N-(benzylsulfonyl)-3-[1-(2,4-dichlorobenzyl)-3-isopropoxy-1H-pyrazol-5-yl]propanamide (620 mg, yield: 87%) as colorless crystals. melting point 183-184° C.

### Example 85

A mixture of 3-[1-(2,4-dichlorobenzyl)-3-isopropoxy-1H-pyrazol-5-yl]propanoic acid (500 mg), N,N′-carbonyldiimidazole (341 mg) and tetrahydrofuran (10 ml) was stirred with heating under reflux for 1 hr. After cooling to room temperature, propane-1-sulfonamide (240 mg) and 1,8-diazabicyclo[5.4.0]undec-7-ene (320 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-1:1, v/v), concentrated, and crystallized from ethyl acetate-hexane to give 3-[1-(2,4-dichlorobenzyl)-3-isopropoxy-1H-pyrazol-5-yl]-N-(propylsulfonyl)propanamide (520 mg, yield: 80%) as colorless crystals. melting point 124-126° C.

### Example 86

A mixture of 3-[1-(2,4-dichlorobenzyl)-3-isopropoxy-1H-pyrazol-5-yl]propanoic acid (370 mg), N,N′-carbonyldiimidazole (253 mg) and tetrahydrofuran (10 ml) was stirred with heating under reflux for 1 hr. After cooling to room temperature, butane-1-sulfonamide (171 mg) and 1,8-diazabicyclo[5.4.0]undec-7-ene (237 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:24-1:2, v/v), concentrated, and crystallized from ethyl acetate-hexane to give N-(butylsulfonyl)-3-[1-(2,4-dichlorobenzyl)-3-isopropoxy-1H-pyrazol-5-yl]propanamide (450 mg, yield: 66%) as colorless crystals. melting point 133-134° C.

### Example 87

A mixture of 3-[1-(2,4-dichlorobenzyl)-3-isopropoxy-1H-pyrazol-5-yl]propanoic acid (500 mg), N,N′-carbonyldiimidazole (341 mg) and tetrahydrofuran (10 ml) was stirred with heating under reflux for 1 hr. After cooling to room temperature, hexane-1-sulfonamide (278 mg) and 1,8-diazabicyclo[5.4.0]undec-7-ene (320 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:24′-3:7, v/v), concentrated, and crystallized from ethyl acetate-hexane to give 3-[1-(2,4-dichlorobenzyl)-3-isopropoxy-1H-pyrazol-5-yl]-N-(hexylsulfonyl)propanamide (340 mg, yield: 48%) as colorless crystals. melting point 103-106° C.

### Example 88

A mixture of 3-[1-(2,4-dichlorobenzyl)-3-isopropoxy-1H-pyrazol-5-yl]propanoic acid (500 mg), N,N′-carbonyldiimidazole (341 mg) and tetrahydrofuran (10 ml) was stirred with heating under reflux for 1 hr. After cooling to room temperature, 2-methylbenzenesulfonamide (288 mg) and 1,8-diazabicyclo[5.4.0]undec-7-ene (320 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:24-3:7, v/v), concentrated, and crystallized from ethyl acetate-hexane to give 3-[1-(2,4-dichlorobenzyl)-3-isopropoxy-1H-pyrazol-5-yl]-N-[(2-methylphenyl)sulfonyl]propanamide (260 mg, yield: 36%) as colorless crystals. melting point 165-166° C.

### Example 89

A mixture of 3-[1-(2,4-dichlorobenzyl)-3-isopropoxy-1H-pyrazol-5-yl]propanoic acid (500 mg), N,N′-carbonyldiimidazole (341 mg) and tetrahydrofuran (10 ml) was stirred with heating under reflux for 1 hr. After cooling to room temperature, propane-2-sulfonamide (207 mg) and 1,8-diazabicyclo[5.4.0]undec-7-ene (320 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-1:2, v/v), concentrated, and crystallized from ethyl acetate-hexane to give 3-[1-(2,4-dichlorobenzyl)-3-isopropoxy-1H-pyrazol-5-yl]-N-(isopropylsulfonyl)propanamide (350 mg, yield: 54%) as colorless crystals. melting point 135-136° C.

### Example 90

A mixture of 3-[1-(2,4-dichlorobenzyl)-3-isopropoxy-1H-pyrazol-5-yl]propanoic acid (500 mg), N,N′-carbonyldiimidazole (341 mg) and tetrahydrofuran (10 ml) was stirred with heating under reflux for 1 hr. After cooling to room temperature, cyclohexanesulfonamide (274 mg) and 1,8-diazabicyclo[5.4.0]undec-7-ene (427 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-1:2, v/v), concentrated, and crystallized from ethyl acetatehexane to give N-(cyclohexylsulfonyl)-3-[1-(2,4-dichlorobenzyl)-3-isopropoxy-1H-pyrazol-5-yl]propanamide (390 mg, yield: 55%) as colorless crystals. melting point 137-138° C.

### Example 91

A mixture of 3-[1-(2,4-dichlorobenzyl)-3-isopropoxy-1H-pyrazol-5-yl]propanoic acid (500 mg), N,N′-carbonyldiimidazole (341 mg) and tetrahydrofuran (10 ml) was stirred with heating under reflux for 1 hr. After cooling to room temperature, 3-phenylpropane-1-sulfonamide (334 mg) and 1,8-diazabicyclo[5.4.0]undec-7-ene (427 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:24-3:7, v/v), concentrated, and crystallized from ethyl acetate-hexane to give 3-[1-(2,4-dichlorobenzyl)-3-isopropoxy-1H-pyrazol-5-yl]-N-[(3-phenylpropyl)sulfonyl]propanamide (570 mg, yield: 76%) as colorless crystals. melting point 132-134° C.

### Example 92

A mixture of 3-[1-(2,4-dichlorobenzyl)-3-isopropoxy-1H-pyrazol-5-yl]propanoic acid (400 mg), N,N′-carbonyldiimidazole (272 mg) and tetrahydrofuran (10 ml) was stirred with heating under reflux for 1 hr. After cooling to room temperature, 3-methylbutyl-1-sulfonamide (203 mg) and 1,8-diazabicyclo[5.4.0]undec-7-ene (256 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-1:2, v/v), concentrated, and crystallized from ethyl acetatehexane to give 3-[1-(2,4-dichlorobenzyl)-3-isopropoxy-1H-pyrazol-5-yl]-N-[(3-methylbutyl)sulfonyl]propanamide (400 mg, yield: 73%) as colorless crystals. melting point 143-145° C.

### Example 93

A mixture of 3-[1-(2,4-dichlorobenzyl)-3-isopropyl-1H-pyrazol-5-yl]propanoic acid (500 mg), N,N′-carbonyldiimidazole (356 mg) and tetrahydrofuran (10 ml) was stirred with heating under reflux for 1 hr. After cooling to room temperature, pentane-1-sulfonamide (267 mg) and 1,8-diazabicyclo[5.4.0]undec-7-ene (336 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-1:1, v/v), concentrated, and crystallized from ethyl acetate-hexane to give 3-[1-(2,4-dichlorobenzyl)-3-isopropyl-1H-pyrazol-5-yl]-N-(pentylsulfonyl)propanamide (470 mg, yield: 67%) as colorless crystals. melting point 115-117° C.

### Example 94

A mixture of (2E)-3-[1-(2,4-dichlorobenzyl)-3-isopropoxy-1H-pyrazol-5-yl]acrylic acid (500 mg), N,N′-carbonyldiimidazole (343 mg) and tetrahydrofuran (10 ml) was stirred with heating under reflux for 1 hr. After cooling to room temperature, pentane-1-sulfonamide (256 mg) and 1,8-diazabicyclo[5.4.0]undec-7-ene (322 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-2:3, v/v), concentrated, and crystallized from ethyl acetate-hexane to give (2E)-3-[1-(2,4-dichlorobenzyl)-3-isopropoxy-1H-pyrazol-5-yl]-N-(pentylsulfonyl)acrylamide (220 mg, yield: 32%) as colorless crystals. melting point 109-112° C.

### Example 95

A mixture of (2E)-3-[1-(2,4-dichlorobenzyl)-3-isopropyl-1H-pyrazol-5-yl]acrylic acid (410 mg), N,N′-carbonyldiimidazole (274 mg) and tetrahydrofuran (10 ml) was stirred with heating under reflux for 1 hr. After cooling to room temperature, pentane-1-sulfonamide (220 mg) and 1,8-diazabicyclo[5.4.0]undec-7-ene (276 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-2:3, v/v), concentrated, and crystallized from ethyl acetate-hexane to give (2E)-3-[1-(2,4-dichlorobenzyl)-3-isopropyl-1H-pyrazol-5-yl]-N-(pentylsulfonyl)acrylamide (140 mg, yields 24%) as colorless crystals. melting point 149-151° C.

### Example 96

A mixture of 3-{1-[2-chloro-4-(trifluoromethyl)benzyl]-3-isopropyl-1H-pyrazol-5-yl}propanoic acid (500 mg), N,N′-carbonyldiimidazole (323 mg) and tetrahydrofuran (10 ml) was stirred with heating under reflux for 1 hr. After cooling to room temperature, pentane-1-sulfonamide (241 mg) and 1,8-diazabicyclo[5.4.0]undec-7-ene (305 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:16-3:7, v/v), concentrated, and crystallized from ethyl acetate-hexane to give 3-{1-[2-chloro-4-(trifluoromethyl)benzyl]-3-isopropyl-1H-pyrazol-5-yl}-N-(pentylsulfonyl)propanamide (260 mg, yield: 38%) as colorless crystals. melting point 144-145° C.

### Example 97

A mixture of 3-[3-butoxy-1-(2,4-dichlorobenzyl)-1H-pyrazol-5-yl]propanoic acid (500 mg), N,N′-carbonyldiimidazole (328 mg) and tetrahydrofuran (10 ml) was stirred with heating under reflux for 1 hr. After cooling to room temperature, pentane-1-sulfonamide (245 mg) and 1,8-diazabicyclo[5.4.0]undec-7-ene (309 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-1:1, v/v), concentrated, and crystallized from ethyl acetate-hexane to give 3-[3-butoxy-1-(2,4-dichlorobenzyl)-1H-pyrazol-5-yl]-N-(pentylsulfonyl)propanamide (450 mg, yield: 66%) as colorless crystals. melting point 104-105° C.

¹H-NMR (300 MHz, CDCl₃) δ:0.85-1.00 (6H, m), 1.24-1.87 (10H, m), 2.53-2.63 (2H, m), 2.75-2.86 (2H, m), 3.33-3.43 (2H, m), 4.08 (2H, t, J=6.6 Hz), 5.19 (2H, s), 5.53 (1H, s), 6.56 (1H, d, J=8.5 Hz), 7.15 (1H, dd, J=8.5, 2.2 Hz), 7.39 (1H, d, J=2.2 Hz), 8.17 (1H, brs).

### Example 98

A mixture of (2E)-3-[3-butoxy-1-(2,4-dichlorobenzyl)-1H-pyrazol-5-yl]acrylic acid (2.50 g), N,N′-carbonyldiimidazole (1.65 g) and tetrahydrofuran (50 ml) was stirred with heating under reflux for 1 hr. After cooling to room temperature, pentane-1-sulfonamide (1.23 g) and 1,8-diazabicyclo[5.4.0]undec-7-ene (1.55 g) were added, the mixture was stirred at room temperature for 15 hr, then stirred with heating under reflux for 2 hr, and concentrated. 1N Hydrochloric acid (100 ml) was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:24-1:2, v/v), concentrated, and crystallized from ethyl acetate-hexane to give (2E)-3-[3-butoxy-1-(2,4-dichlorobenzyl)-1H-pyrazol-5-yl]-N-(pentylsulfonyl)acrylamide (1.01 g, yield: 30%) as colorless crystals. melting point 95-98° C.

### Example 99

To a solution of (2E)-3-[3-butoxy-1-(2,4-dichlorobenzyl)-1H-pyrazol-5-yl]acrylic acid (820 mg) in tetrahydrofuran (15 ml) were added thionyl chloride (528 mg) and N,N-dimethylformamide (20 mg) under ice-cooling, and the mixture was stirred for 1 hr. The reaction mixture was concentrated. To a solution of the residue in tetrahydrofuran (15 ml) were added 3-methylbutane-1-sulfonamide (403 mg), N,N-diisopropylethylamine (861 mg) and 4-dimethylaminopyridine (271 mg) under ice-cooling, and the mixture was stirred at room temperature for 15 hr and concentrated. 1N Hydrochloric acid (100 ml) was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-3:7, v/v), concentrated, and crystallized from ethyl acetate-hexane to give (2E)-3-[3-butoxy-1-(2,4-dichlorobenzyl)-1H-pyrazol-5-yl]-N-[(3-methylbutyl)sulfonyl]acrylamide (540 mg, yield: 48%) as colorless crystals. melting point 119-120° C.

### Example 100

A mixture of 3-[1-(2,4-dichlorobenzyl)-3-phenyl-1H-pyrazol-5-yl]propanoic acid (2.00 g), N,N′-carbonyldiimidazole (1.30 g) and tetrahydrofuran (35 ml) was stirred with heating under reflux for 1 hr. After cooling to room temperature, pentane-1-sulfonamide (967 mg) and 1,8-diazabicyclo[5.4.0]undec-7-ene (1.22 g) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:100-1:2, v/v), concentrated, and crystallized from ethyl acetate-hexane to give 3-[1-(2,4-dichlorobenzyl)-3-phenyl-1H-pyrazol-5-yl]-N-(pentylsulfonyl)propanamide (290 mg, yield: 11%) as colorless crystals. melting point 145-147° C.

### Example 101

A mixture of 3-[1-(2,4-dichlorobenzyl)-3-(2-methoxyethoxy)-1H-pyrazol-5-yl]propanoic acid (750 mg), N,N′-carbonyldiimidazole (489 mg) and tetrahydrofuran (20 ml) was stirred with heating under reflux for 1 hr. After cooling to room temperature, pentane-1-sulfonamide (365 mg) and 1,8-diazabicyclo[5.4.0]undec-7-ene (459 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-2:3, v/v), concentrated, and crystallized from ethyl acetate-hexane to give 3-[1-(2,4-dichlorobenzyl)-3-(2-methoxyethoxy)-1H-pyrazol-5-yl]-N-(pentylsulfonyl)propanamide (310 mg, yield: 30%) as colorless crystals. melting point 108-112° C.

### Example 102

A mixture of 3-[3-(benzyloxy)-1-(2,4-dichlorobenzyl)-1H-pyrazol-5-yl]propanoic acid (400 mg), N,N′-carbonyldiimidazole (240 mg) and tetrahydrofuran (10 ml) was stirred with heating under reflux for 1 hr. After cooling to room temperature, pentane-1-sulfonamide (179 mg) and 1,8-diazabicyclo[5.4.0]undec-7-ene (226 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:25-1:2, v/v), concentrated, and crystallized from ethyl acetate-hexane to give 3-[3-(benzyloxy)-1-(2,4-dichlorobenzyl)-1H-pyrazol-5-yl]-N-(pentylsulfonyl)propanamide (300 mg, yield: 56%) as colorless crystals. melting point 139-141° C.

### Example 103

Under ice-cooling, to a solution of (2E)-3-{3-butoxy-1-[4-(trifluoromethyl)benzyl]-1H-pyrazol-5-yl}acrylic acid (730 mg) in tetrahydrofuran (7.0 ml) were successively added thionyl chloride (471 mg) and N,N-dimethylformamide (2.0 mg), and the mixture was stirred for 1 hr. The reaction mixture was concentrated. The residue was dissolved in tetrahydrofuran (10 ml), and pentane-1-sulfonamide (179 mg), N,N-diisopropylethylamine (768 mg) and 4-dimethylaminopyridine (242 mg) were added under ice-cooling. The mixture was allowed to warm to room temperature, stirred for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:12-1:1, v/v), concentrated, and crystallized from ethyl acetate-hexane to give (2E)-3-{3-butoxy-1-[4-(trifluoromethyl)benzyl]-1H-pyrazol-5-yl}-N-(pentylsulfonyl)acrylamide (250 mg, yield: 25%) as colorless crystals. melting point 159-160° C.

### Example 104

A mixture of 3-{3-butoxy-1-[4-(trifluoromethyl)benzyl]-1H-pyrazol-5-yl}propanoic acid (680 mg), N,N′-carbonyldiimidazole (448 mg) and tetrahydrofuran (12 ml) was stirred with heating under reflux for 1 hr. After cooling to room temperature, pentane-1-sulfonamide (334 mg) and 1,8-diazabicyclo[5.4.0]undec-7-ene (421 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-1:2, v/v) to give 3-{3-butoxy-1-[4-(trifluoromethyl)benzyl]-1H-pyrazol-5-yl}-N-(pentylsulfonyl)propanamide (170 mg, yield: 18%) as a colorless oil.

¹H-NMR (300 MHz, CDCl₃) δ:0.88 (3H, t, J=7.1 Hz), 0.94 (3H, t, J=7.4 Hz), 1.23-1.51 (6H, m), 1.63-1.80 (4H, m), 2.50-2.62 (2H, m), 2.71-2.83 (2H, m), 3.27-3.40 (2H, m), 4.06 (2H, t, J=6.6 Hz), 5.21 (2H, s), 5.51 (1H, s), 7.17 (2H, d, J=8.3 Hz), 7.55 (2H, d, J=8.3 Hz).

### Example 105

A mixture of 3-{1-(2,4-dichlorobenzyl)-3-[(5-methyl-1,3,4-oxadiazol-2-yl)methoxy]-1H-pyrazol-5-yl}propanoic acid (250 mg), N,N′-carbonyldiimidazole (148 mg) and tetrahydrofuran (5.0 ml) was stirred with heating under reflux for 1 hr. After cooling to room temperature, pentane-1-sulfonamide (97 mg) and 1,8-diazabicyclo[5.4.0]undec-7-ene (139 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:12-2:1, v/v), ethyl acetate and methanol-chloroform (1:16-1:7, v/v), concentrated and crystallized from ethyl acetate-hexane to give 3-{1-(2,4-dichlorobenzyl)-3-[(5-methyl-1,3,4-oxadiazol-2-yl)methoxy]-1H-pyrazol-5-yl}-N-(pentylsulfonyl)propanamide (210 mg, yield: 63%) as colorless crystals. melting point 156-158° C.

### Example 106

To a solution of (2E)-3-{3-butoxy-1-[2-chloro-4-(trifluoromethyl)benzyl]-1H-pyrazol-5-yl}acrylic acid (690 mg) in tetrahydrofuran (7.0 ml) were added thionyl chloride (407 mg) and N,N-dimethylformamide (20 mg) under ice-cooling, and the mixture was stirred for 1 hr. The reaction mixture was concentrated. To a solution of the residue in tetrahydrofuran (10 ml) were added pentane-1-sulfonamide (272 mg), N,N-diisopropylethylamine (663 mg) and 4-dimethylaminopyridine (209 mg) under ice-cooling, and the mixture was stirred at room temperature for 15 hr and concentrated. 1N Hydrochloric acid (100 ml) was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:12-1:1, v/v), concentrated, and crystallized from ethyl acetate-hexane to give (2E)-3-{3-butoxy-1-[2-chloro-4-(trifluoromethyl)benzyl]-1H-pyrazol-5-yl}-N-(pentylsulfonyl)acrylamide (370 mg, yield: 40%) as colorless crystals. melting point 130-132° C.

### Example 107

A mixture of 3-{3-butoxy-1-[2-chloro-4-(trifluoromethyl)benzyl]-1H-pyrazol-5-yl}propanoic acid (600 mg), N,N′-carbonyldiimidazole (360 mg) and N,N-dimethylformamide (15 ml) was stirred at room temperature for 1 hr, pentane-1-sulfonamide (269 mg) and 1,8-diazabicyclo[5.4.0]undec-7-ene (338 mg) were added, and the mixture was stirred at 100° C. for 15 hr. After cooling. to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-1:2, v/v), concentrated, and crystallized from ethyl acetate-hexane to give 3-{3-butoxy-1-[2-chloro-4-(trifluoromethyl)benzyl]-1H-pyrazol-5-yl}-N-(pentylsulfonyl)propanamide (320 mg, yield: 40%) as colorless crystals. melting point 131-133° C.

### Example 108

A mixture of 3-[1-(2,4-dichlorobenzyl)-3-(pyridin-2-ylmethoxy)-1H-pyrazol-5-yl]propanoic acid (760 mg), N,N′-carbonyldiimidazole (455 mg) and N,N-dimethylformamide (15 ml) was stirred with heating under reflux for 1 hr. After cooling to room temperature, pentane-1-sulfonamide (297 mg) and 1,8-diazabicyclo[5.4.0]undec-7-ene (428 mg) were added. The mixture was stirred at room temperature for 15 hr, and concentrated. 1N Hydrochloric acid (200 ml) and ethyl acetate (20 ml) were added to the concentrate, and the resulting precipitate was collected by filtration to give 3-[1-(2,4-dichlorobenzyl)-3-(pyridin-2-ylmethoxy)-1H-pyrazol-5-yl]-N-(pentylsulfonyl)propanamide hydrochloride (500 mg, yield: 46%) as a white solid.

¹H-NMR (300 MHz, CDCl₃) δ:0.81 (3H, t, J=7.2 Hz), 1.14-1.38 (4H, m), 1.52-1.67 (2H, m), 2.57-2.67 (2H, m), 2.71-2.83 (2H, m), 3.27-3.37 (2H, m), 5.20 (2H, s), 5.28 (2H, s), 5.71 (1′H, s), 6.63 (1H, d, J=8.4 Hz), 7.35 (1H, dd, J=2.1, 8.4 Hz), 7.56-7.74 (3H, m), 8.08-8.18 (1H, m), 8.67 (1H, d, J=4.8 Hz), 11.71 (1H, m)

### Example 109

A mixture of (2E)-3-[1-(2,4-dichlorobenzyl)-3-(2-methoxyethoxy)-1H-pyrazol-5-yl]acrylic acid (520 mg), N,N′-carbonyldiimidazole (341 mg) and N,N-dimethylformamide (10 ml) was stirred at room temperature for 1 hr, pentane-1-sulfonamide (254 mg) and 1,8-diazabicyclo[5.4.0]undec-7-ene (320 mg) were added, and the mixture was stirred at 100° C. for 15 hr. After cooling to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-1:1, v/v), concentrated, and crystallized from ethyl acetate-hexane to give (2E)-3-[1-(2,4-dichlorobenzyl)-3-(2-methoxyethoxy)-1H-pyrazol-5-yl]-N-(pentylsulfonyl)acrylamide (25 mg, yield: 4%) as colorless crystals. melting point 82-85° C.

### Example 110

A mixture of (2E)-3-[1-(2,4-dichlorobenzyl)-3-(2-methoxyethoxy)-1H-pyrazol-5-yl]acrylic acid (520 mg), N,N′-carbonyldiimidazole (341 mg) and N,N-dimethylformamide (10 ml) was stirred at room temperature for 1 hr, 3-methylbutane-1-sulfonamide (254 mg) and 1,8-diazabicyclo[5.4.0]undec-7-ene (320 mg) were added, and the mixture was stirred at 100° C. for 15 hr. After cooling to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-1:1, v/v), concentrated, and crystallized from ethyl acetate-hexane to give (2E)-3-[1-(2,4-dichlorobenzyl)-3-(2-methoxyethoxy)-1H-pyrazol-5-yl]-N-[(3-methylbutyl)sulfonyl]acrylamide (310 mg, yield: 44%) as colorless crystals. melting point 110-114° C.

### Example 111

A mixture of (2E)-3-{1-[2-chloro-4-(trifluoromethyl)benzyl]-3-isopropoxy-1H-pyrazol-5-yl}acrylic acid (600 mg), N,N′-carbonyldiimidazole (375 mg) and N,N-dimethylformamide (20 ml) was stirred at room temperature for 1 hr, pentane-1-sulfonamide (279 mg) and 1,8-diazabicyclo[5.4.0]undec-7-ene (352 mg) were added, and the mixture was stirred at 100° C. for 15 hr. After cooling to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-1:2, v/v), concentrated, and crystallized from ethyl acetate-hexane to give (2E)-3-{1-[2-chloro-4-(trifluoromethyl)benzyl]-3-isopropoxy-1H-pyrazol-5-yl}-N-(pentylsulfonyl)acrylamide (250 mg, yield: 44%) as colorless crystals. melting point 139-140° C.

### Example 112

A mixture of (2E)-3-{1-[2-chloro-4-(trifluoromethyl)benzyl]-3-isopropoxy-1H-pyrazol-5-yl}acrylic acid (600 mg), N,N′-carbonyldiimidazole (375 mg) and N,N-dimethylformamide (20 ml) was stirred at room temperature for 1 hr, 3-methylbutane-1-sulfonamide (279 mg) and 1,8-diazabicyclo[5.4.0]undec-7-ene (352 mg) were added, and the mixture was stirred at 100° C. for 15 hr. After cooling to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-1:2, v/v), concentrated, and crystallized from ethyl acetate-hexane to give (2E)-3-{1-[2-chloro-4-(trifluoromethyl)benzyl]-3-isopropoxy-1H-pyrazol-5-yl}-N-[(3-methylbutyl)sulfonyl]acrylamide (250 mg, yield: 44%) as colorless crystals. melting point 139-140° C.

### Example 113

A mixture of (2E)-3-{1-[2-chloro-4-(trifluoromethyl)benzyl]-3-[(3-methyloxetan-3-yl)methoxy]-1H-pyrazol-5-yl}acrylic acid (460 mg), N,N′-carbonyldiimidazole (226 mg) and N,N-dimethylformamide (15 ml) was stirred at room temperature for 1 hr, pentane-1-sulfonamide (169 mg) and 1,8-diazabicyclo[5.4.0]undec-7-ene (213 mg) were added, and the mixture was stirred at 100° C. for 15 hr. After cooling to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-4:1, v/v), concentrated, and crystallized from ethyl acetate-hexane to give (2E)-3-{1-[2-chloro-4-(trifluoromethyl)benzyl]-3-[(3-methyloxetan-3-yl)methoxy]-1H-pyrazol-5-yl}-N-(pentylsulfonyl)acrylamide (200 mg, yield: 38%) as colorless crystals. melting point 144-147° C.

### Example 114

A mixture of (2E)-3-[1-(2,4-dichlorobenzyl)-3-isopropoxy-1H-pyrazol-5-yl]acrylic acid (1.00 g), N,N′-carbonyldiimidazole (686 mg) and N,N-dimethylformamide (25 ml) was stirred at room temperature for 1 hr, 3-methylbutane-1-sulfonamide (512 mg) and 1,8-diazabicyclo[5.4.0]undec-7-ene (645 mg) were added, and the mixture was stirred at 100° C. for 15 hr. After cooling to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-3:7, v/v), concentrated, and crystallized from ethyl acetate-hexane to give (2E)-3-[1-(2,4-dichlorobenzyl)]-3-isopropoxy-1H-pyrazol-5-yl}-N-[(3-methylbutyl)sulfonyl]acrylamide (380 mg, yield: 28%) as colorless crystals. melting point 132-135° C.

### Example 115

A mixture of (2E)-3-{1-[2-chloro-4-(trifluoromethyl)benzyl]-3-isopropoxy-1H-pyrazol-5-yl}acrylic acid (640 mg), N,N′-carbonyldiimidazole (401 mg) and N,N-dimethylformamide (20 ml) was stirred at room temperature for 1 hr, 3-methoxypropane-1-sulfonamide (303 mg) and 1,8-diazabicyclo[5.4.0]undec-7-ene (378 mg) were added, and the mixture was stirred at 100° C. for 4 hr. After cooling to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-2:3, v/v), concentrated, and crystallized from ethyl acetate-hexane to give (2E)-3-{1-[2-chloro-4-(trifluoromethyl)benzyl]-3-isopropoxy-1H-pyrazol-5-yl}-N-[(3-methoxypropyl)sulfonyl]acrylamide (510 mg, yield: 59%) as colorless crystals. melting point 138-139° C.

### Example 116

To a solution of (2E)-3-[1-(2,4-dichlorobenzyl)-3-(tetrahydro-2H-pyran-4-yloxy)-1H-pyrazol-5-yl]acrylic acid (660 mg) in tetrahydrofuran (10 ml) were added thionyl chloride (395 mg) and N,N-dimethylformamide (20 mg) under ice-cooling, and the mixture was stirred for 1 hr. The reaction mixture was concentrated. To a solution of the residue in tetrahydrofuran (10 ml) were added pentane-1-sulfonamide (301 mg), N,N-diisopropylethylamine (644 mg) and 4-dimethylaminopyridine (203 mg) under ice-cooling, and the mixture was stirred at room temperature for 15 hr and concentrated. 1N Hydrochloric acid (100 ml) was added to the residue, and the mixture was extracted with ethyl acetate. The extract was washed with saturated brine, dried (MgSO₄), and concentrated. The residue was subjected to silica gel column chromatography, and eluted with ethyl acetate-hexane (1:19-1:1, v/v), concentrated, and crystallized from ethyl acetate-hexane to give (2E)-3-[1-(2,4-dichlorobenzyl)-3-(tetrahydro-2H-pyran-4-yloxy)-1H-pyrazol-5-yl]-N-(pentylsulfonyl)acrylamide (160 mg, yield: 18%) as pale-yellow crystals. melting point 154-157° C.

### Example 117

A mixture of (2E)-3-[1-(2,4-dichlorobenzyl)-3-(tetrahydro-2H-pyran-4-yloxy)-1H-pyrazol-5-yl]acrylic acid (660 mg), N,N′-carbonyldiimidazole (404 mg) and N,N-dimethylformamide (20 ml) was stirred at room temperature for 1 hr, 3-methylbutane-1-sulfonamide (301 mg) and 1,8-diazabicyclo[5.4.0]undec-7-ene (380 mg) were added, and the mixture was stirred at 100° C. for 15 hr. After cooling to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-3:7, v/v), concentrated, and crystallized from ethyl acetate-hexane to give (2E)-3-[1-(2,4-dichlorobenzyl)-3-(tetrahydro-2H-pyran-4-yloxy)-1H-pyrazol-5-yl]-N-[(3-methylbutyl)sulfonyl]acrylamide (320 mg, yield: 36%) as colorless crystals. melting point 143-144° C.

### Example 118

A mixture of (2E)-3-{1-[2-chloro-4-(trifluoromethyl)benzyl]-3-cyclopropyl-1H-pyrazol-5-yl}acrylic acid (660 mg), N,N′-carbonyldiimidazole (426 mg) and N,N-dimethylformamide (20 ml) was stirred at room temperature for 1 hr, 3-methylbutane-1-sulfonamide (318 mg) and 1,8-diazabicyclo[5.4.0]undec-7-ene (401 mg) were added, and the mixture was stirred at 100° C. for 15 hr. After cooling to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-1:1, v/v), concentrated, and crystallized from ethyl acetate-hexane to give (2E)-3-{1-[2-chloro-4-(trifluoromethyl)benzyl]-3-cyclopropyl-1H-pyrazol-5-yl}-N-[(3-methylbutyl)sulfonyl]acrylamide (480 mg, yield: 54%) as colorless crystals. melting point 145-147° C.

### Example 119

A mixture of (2E)-3-[1-[2-chloro-4-(trifluoromethyl)benzyl]-3-(cyclopropylmethoxy)-1H-pyrazol-5-yl]acrylic acid (300 mg), N,N′-carbonyldiimidazole (182 mg) and N,N-dimethylformamide (8.0 ml) was stirred at room temperature for 1 hr, 3-methoxypropane-1-sulfonamide (138 mg) and 1,8-diazabicyclo[5.4.0]undec-7-ene (171 mg) were added, and the mixture was stirred at 100° C. for 15 hr. After cooling to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-1:1, v/v), concentrated, and crystallized from ethyl acetate-hexane to give (2E)-3-[1-[2-chloro-4-(trifluoromethyl)benzyl]-3-(cyclopropylmethoxy)-1H-pyrazol-5-yl]-N-[(3-methoxypropyl)sulfonyl]acrylamide (260 mg, yield: 65%) as colorless crystals. melting point 137-138° C.

### Example 120

A mixture of 3-[1-[2z-chloro-4-(trifluoromethyl)benzyl]-3-(cyclopropylmethoxy)-1H-pyrazol-5-yl]propanoic acid (430 mg), N,N′-carbonyldiimidazole (260 mg) and tetrahydrofuran (10 ml) was stirred with heating under reflux for 1 hr. After cooling to room temperature, 3-methoxypropane-1-sulfonamide (197 mg) and 1,8-diazabicyclo[5.4.0]undec-7-ene (244 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-1:1, v/v), concentrated, and crystallized from ethyl acetate-hexane to give 3-[1-[2-chloro-4-(trifluoromethyl)benzyl]-3-(cyclopropylmethoxy)-1H-pyrazol-5-yl]-N-[(3-methoxypropyl)sulfonyl]propanamide (390 mg, yield: 68%) as colorless crystals. melting point 152-154° C.

### Example 121

A mixture of (2E)-3-{3-tert-butyl-1-[2-chloro-4-(trifluoromethyl)benzyl]-1H-pyrazol-5-yl}acrylic acid (840 mg), N,N′-carbonyldiimidazole (528 mg) and N,N-dimethylformamide (20 ml) was stirred at room temperature for 1 hr, pentane-1-sulfonamide (345 mg) and 1,8-diazabicyclo[5.4.0]undec-7-ene (497 mg) were added, and the mixture was stirred at 100° C. for 15 hr. After cooling to room temperature, the reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:19-1:2, v/v), concentrated, and crystallized from ethyl acetate-hexane to give (2E)-3-{3-tert-butyl-1-[2-chloro-4-(trifluoromethyl)benzyl]-1H-pyrazol-5-yl}-N-(pentylsulfonyl)acrylamide (400 mg, yield: 35%) as colorless crystals. melting point 147-148° C.

### Example 122

A mixture of 3-{3-tert-butyl-1-[2-chloro-4-(trifluoromethyl)benzyl]-1H-pyrazol-5-yl}propanoic acid (700 mg), N,N′-carbonyldiimidazole (408 mg) and tetrahydrofuran (15 ml) was stirred with heating under reflux for 1 hr. After cooling to room temperature, pentane-1-sulfonamide (286 mg) and 1,8-diazabicyclo[5.4.0]undec-7-ene (412 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-2:1, v/v), concentrated, and crystallized from ethyl acetate-hexane to give 3-{3-tert-butyl-1-[2-chloro-4-(trifluoromethyl)benzyl]-1H-pyrazol-5-yl}-N-(pentylsulfonyl)propanamide (500 mg, yield: 53%) as colorless crystals. melting point 136-137° C.

¹H-NMR (300 MHz, CDCl₃) δ:0.90 (3H, t, J=7.1 Hz), 1.23-1.47 (13H, m), 1.70-1.85 (2H, m), 2.57-2.68 (2H, m), 2.77-2.87 (2H, m), 3.34-3.43 (2H, m), 5.39 (2H, s), 5.99 (1H, s), 6.50 (1H, d, J=8.1 Hz), 7.42 (1H, d, J=8.1 Hz), 7.65 (1H, s), 8.00 (1H, brs).

### Example 123

A mixture of 3-{3-tert-butyl-1-[2-chloro-4-(trifluoromethyl)benzyl]-1H-pyrazol-5-yl}propanoic acid (500 mg), N,N′-carbonyldiimidazole (313 mg) and tetrahydrofuran (15 ml) was stirred with heating under reflux for 1 hr. After cooling to room temperature, 3-methoxypropane-1-sulfonamide (237 mg) and 1,8-diazabicyclo[5.4.0]undec-7-ene (296 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:19-7:3, v/v), concentrated, and crystallized from ethyl acetate-hexane to give 3-{3-tert-butyl-1-[2-chloro-4-(trifluoromethyl)benzyl]-1H-pyrazol-5-yl}-N-[(3-methoxypropyl)sulfonyl]propanamide (370 mg, yield: 55%) as colorless crystals. melting point 149-150° C.

### Example 124

Under ice-cooling, to a solution of tert-butyl {[(diphenylphosphoryl)methyl]sulfonyl}carbamate (1.89 g) in N,N-dimethylformamide (20 ml) was added sodium hydride (60% in oil, 479 mg), and the mixture was stirred at room temperature for 30 min. The reaction mixture was immersed in an ice bath, 1-(2,4-dichlorobenzyl)-3-isopropoxy-1H-pyrazole-5-carbaldehyde (1.00 g) was added, and the mixture was stirred for 1 hr. The reaction mixture was poured into saturated brine, and the mixture was extracted with ethyl acetate. The extract was washed with saturated brine, dried (MgSO₄), and concentrated. The residue was subjected to silica gel column chromatography, and eluted with ethyl acetate-hexane (1:19-3:7, v/v), concentrated, and crystallized from ethyl acetate-hexane to give tert-butyl ({(E)-2-[1-(2,4-dichlorobenzyl)-3-isopropoxy-1H-pyrazol-5-yl]vinyl}sulfonyl)carbamate (610 mg, yield: 39%) as colorless crystals. melting point 152-153° C.

### Example 125

A mixture of hexanoic acid (107 mg), N,N′-carbonyldiimidazole (299 mg) and tetrahydrofuran (6 ml) was stirred with heating under reflux for 1 hr. After cooling to room temperature, (E)-2-[1-(2,4-dichlorobenzyl)-3-isopropoxy-1H-pyrazol-5-yl]ethylenesulfonamide (300 mg) and 1,8-diazabicyclo[5.4.0]undec-7-ene (305 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:24-3:7, v/v), concentrated, and crystallized from ethyl acetate-hexane to give N-({(E)-2-[1-(2,4-dichlorobenzyl)-3-isopropoxy-1H-pyrazol-5-yl]vinyl}sulfonyl)hexanamide (110 mg, yield: 29%) as colorless crystals. melting point 73-75° C.

### Example 126

A mixture of hexanoic acid (98 mg), N,N′-carbonyldiimidazole (273 mg) and tetrahydrofuran (4 ml) was stirred with heating under reflux for 1 hr. After cooling to room temperature, 2-[1-(2,4-dichlorobenzyl)-3-isopropoxy-1H-pyrazol-5-yl]ethanesulfonamide (220 mg) and 1,8-diazabicyclo[5.4.0]undec-7-ene (256 mg) were added. The mixture was stirred at room temperature for 15 hr, and concentrated. 1N Hydrochloric acid was added to the concentrate, and the mixture was extracted with ethyl acetate. The extract was washed with saturated brine, dried (MgSO₄), and concentrated. The residue was subjected to silica gel column chromatography, and eluted with ethyl acetate-hexane (1:24-3:7, v/v), concentrated, and crystallized from ethyl acetate-hexane to give N-({2-[1-(2,4-dichlorobenzyl)-3-isopropoxy-1H-pyrazol-5-yl]ethyl}sulfonyl)hexanamide (25 mg, yield: 9%) as colorless crystals. melting point 107-108° C.

### Example 127

Under ice-cooling, to a solution of: tert-butyl {[(diphenylphosphoryl)methyl]sulfonyl}carbamate (7.24 g) in N,N-dimethylformamide (120 ml) was added sodium hydride (60% in oil, 479 mg), and the mixture was stirred at room temperature for 30 min. The reaction mixture was cooled with an ice bath, 3-butoxy-1-(2,4-dichlorobenzyl)-1H-pyrazole-5-carbaldehyde (4.00 g) was added, and the mixture was stirred for 1 hr. The reaction mixture was poured into saturated brine, and the mixture was extracted with ethyl acetate. The extract was washed with saturated brine, dried (MgSO₄), and concentrated. The residue was subjected to silica gel column chromatography, and eluted with ethyl acetate-hexane (1:19-1:2, v/v), concentrated, and crystallized from ethyl acetate-hexane to give tert-butyl ({(E)-2-[3-butoxy-1-(2,4-dichlorobenzyl)-1H-pyrazol-5-yl]vinyl}sulfonyl)carbamate (610 mg, yield: 39%) as colorless crystals. melting point 152-153° C.

### Example 128

Under ice-cooling, to a mixture of 2-[3-butoxy-1-(2,4-dichlorobenzyl)-1H-pyrazol-5-yl]ethanesulfonamide (500 mg), 4-pyrrolidinopyridine (182 mg) and pyridine (10 ml) was added hexanoyl chloride (497 mg), and the mixture was stirred at room temperature for 20 hr. The reaction mixture was poured into 1N hydrochloric acid, and the mixture was extracted with ethyl acetate. The extract was washed with 1N hydrochloric acid, saturated aqueous sodium hydrogencarbonate solution, saturated brine, dried (MgSO₄), and concentrated. To a mixture of the residue, 4-dimethylaminopyridine (299 mg), N,N-diisopropylethylamine (318 mg), and N,N-dimethylacetamide (10 ml) was added hexanoyl chloride (331 mg) under ice-cooling, and the mixture was stirred at room temperature for 15 hr. The reaction mixture was poured into water, and the mixture was extracted with ethyl acetate. The extract was washed with 1N hydrochloric acid, water and saturated brine, dried (MgSO₄), and concentrated. The residue was subjected to silica gel column chromatography, and eluted with ethyl acetate-hexane (1:24-3:7, v/v), concentrated, and crystallized from ethyl acetate-hexane to give N-({2-[3-butoxy-1-(2,4-dichlorobenzyl)-1H-pyrazol-5-yl]ethyl}sulfonyl)hexanamide (250 mg, yield: 40%) as colorless crystals. melting point 90-92° C.

### Example 129

Under ice-cooling, to a mixture of 2-[3-butoxy-1-(2,4-dichlorobenzyl)-1H-pyrazol-5-yl]ethanesulfonamide (500 mg), 4-dimethylaminopyridine (299 mg), N,N-diisopropylethylamine (318 mg) and N,N-dimethylacetamide (10 ml) was added butyl chloroformate (336 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:24-3:7, v/v), concentrated, and crystallized from ethyl acetate-hexane to give butyl ({2-[3-butoxy-1-(2,4-dichlorobenzyl)-1H-pyrazol-5-yl]ethyl}sulfonyl)carbamate (420 mg, yield: 67%) as colorless crystals. melting point 108-111° C.

¹H-NMR (300 MHz, CDCl₃) δ:0.94 (3H, t, J=7.2 Hz), 0.95 (3H, t, J=7.2 Hz), 1.30-1.80 (8H, m), 2.97-3.07 (2H, m), 3.58-3.68 (2H, m), 4.08 (2H, t, J=6.6 Hz), 4.17 (2H, t, J=6.6 Hz), 5.18 (2H, s), 5.60 (1H, s), 6.61 (1H, d, J=8.3 Hz), 7.16 (1H, dd, J=8.3, 2.1 Hz), 7.39 (1H, d, J=2.1 Hz).

### Example 130

Under ice-cooling, to a mixture of (E)-2-[1-(2,4-dichlorobenzyl)-3-isopropoxy-1H-pyrazol-5-yl]ethylenesulfonamide (1.00 g), 4-dimethylaminopyridine (604 mg), N,N-diisopropylethylamine (640 mg) and N,N-dimethylacetamide (20 ml) was added butyl chloroformate (676 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, eluted with ethyl acetate-hexane (1:24-1:3, v/v), concentrated, and crystallized from ethyl acetate-hexane to give butyl ({(E)-2-[3-butoxy-1-(2,4-dichlorobenzyl)-1H-pyrazol-5-yl]vinyl}sulfonyl)carbamate (470 mg, yield: 38%) as colorless crystals. melting point 137-138° C.

### Example 131

Under ice-cooling, to a mixture of (E)-2-[1-(2,4-dichlorobenzyl)-3-butoxy-1H-pyrazol-5-yl]ethylenesulfonamide (1.00 g), 4-dimethylaminopyridine (604 mg), N,N-diisopropylethylamine (640 mg) and N,N-dimethylacetamide (20 ml) was added hexanoyl chloride (666 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, eluted with ethyl acetate-hexane (1:24-3:7, v/v), concentrated, and crystallized from ethyl acetate-hexane to give N-({(E)-2-[3-butoxy-1-(2,4-dichlorobenzyl)-1H-pyrazol-5-yl]vinyl}sulfonyl)hexanamide (830 mg, yield: 67%) as colorless crystals. melting point 117-119° C.

### Example 132

Under ice-cooling, to a mixture of 2-[3-butoxy-1-(2,4-dichlorobenzyl)-1H-pyrazol-5-yl]ethanesulfonamide (300 mg), 4-dimethylaminopyridine (181 mg), N,N-diisopropylethylamine (191 mg) and N,N-dimethylacetamide (5 ml) was added propyl chloroformate (181 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, eluted with ethyl acetate-hexane (1:19-3:7, v/v), concentrated, and crystallized from ethyl acetate-hexane to give propyl ({2-[3-butoxy-1-(2,4-dichlorobenzyl)-1H-pyrazol-5-yl]ethyl}sulfonyl)carbamate (250 mg, yield: 69%) as colorless crystals. melting point 92-94° C.

### Example 133

Under ice-cooling, to a mixture of 2-[3-butoxy-1-(2,4-dichlorobenzyl)-1H-pyrazol-5-yl]ethanesulfonamide (300 mg), 4-dimethylaminopyridine (181 mg), N,N-diisopropylethylamine (191 mg) and N,N-dimethylacetamide (5 ml) was added isobutyl chloroformate (202 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, eluted with ethyl acetate-hexane (1:19-3:7, v/v), concentrated, and crystallized from ethyl acetate-hexane to give isobutyl ({2-[3-butoxy-1-(2,4-dichlorobenzyl)-1H-pyrazol-5-yl]ethyl}sulfonyl)carbamate (280 mg, yield: 75%) as colorless crystals. melting point 114-116° C.

### Example 134

Under ice-cooling, to a mixture of 2-[3-butoxy-1-(2,4-dichlorobenzyl)-1H-pyrazol-5-yl]ethanesulfonamide (300 mg), 4-dimethylaminopyridine (181 mg), N,N-diisopropylethylamine (191 mg) and N,N-dimethylacetamide (5 ml) was added 2-methoxyethyl chloroformate (205 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, eluted with ethyl acetate-hexane (1:19-3:7, v/v), concentrated, and crystallized from ethyl acetate-hexane to give 2-metoxyethyl ({2-[3-butoxy-1-(2,4-dichlorobenzyl)-1H-pyrazol-5-yl]ethyl}sulfonyl)carbamate (280 mg, yield: 75%) as colorless crystals. melting point 82-86° C.

### Example 135

Under ice-cooling, to a mixture of 2-[3-butoxy-1-(2,4-dichlorobenzyl)-1H-pyrazol-5-yl]ethanesulfonamide (300 mg), 4-dimethylaminopyridine (181 mg), N,N-diisopropylethylamine (191 mg) and N,N-dimethylacetamide (5 ml) was added pentyl chloroformate (223 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, eluted with ethyl acetate-hexane (1:19-3:7, v/v), concentrated, and crystallized from ethyl acetate-hexane to give pentyl ({2-[3-butoxy-1-(2,4-dichlorobenzyl)-1H-pyrazol-5-yl]ethyl}sulfonyl)carbamate (250 mg, yield: 65%) as colorless crystals. melting point 91-93° C.

### Example 136

Under ice-cooling, to a mixture of 2-[3-butoxy-1-(2,4-dichlorobenzyl)-1H-pyrazol-5-yl]ethanesulfonamide (300 mg), 4-dimethylaminopyridine (181 mg), N,N-diisopropylethylamine (191 mg) and N,N-dimethylacetamide (5 ml) was added benzyl chloroformate (252 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, eluted with ethyl acetate-hexane (1:19-3:7, v/v), concentrated, and crystallized from ethyl acetate-hexane to give benzyl ({2-[3-butoxy-1-(2,4-dichlorobenzyl)-1H-pyrazol-5-yl]ethyl}sulfonyl)carbamate (130 mg, yield: 33%) as colorless crystals. melting point 99-101° C.

### Example 137

Under ice-cooling, to a mixture of 2-[3-butoxy-1-(2,4-dichlorobenzyl)-1H-pyrazol-5-yl]ethanesulfonamide (300 mg), 4-dimethylaminopyridine (181 mg), N,N-diisopropylethylamine (191 mg) and N,N-dimethylacetamide (5 ml) was added allyl chloroformate (178 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:24-3:7, v/v), concentrated, and crystallized from ethyl acetate-hexane to give allyl ({2-[3-butoxy-1-(2,4-dichlorobenzyl)-1H-pyrazol-5-yl]ethyl}sulfonyl)carbamate (200 mg, yield: 55%) as colorless crystals. melting point 94-95° C.

### Example 138

Under ice-cooling, to a mixture of 2-[3-butoxy-1-(2,4-dichlorobenzyl)-1H-pyrazol-5-yl]ethanesulfonamide (300 mg), 4-dimethylaminopyridine (181 mg), N,N-diisopropylethylamine (191 mg) and N,N-dimethylacetamide (5 ml) was added hexyl chloroformate (178 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:24-3:7, v/v), concentrated, and crystallized from ethyl acetate-hexane to give benzyl ({2-[3-butoxy-1-(2,4-dichlorobenzyl)-1H-pyrazol-5-yl]ethyl}sulfonyl)carbamate (160 mg, yield: 41%) as colorless crystals. melting point 92-95° C.

### Example 139

Under ice-cooling, to a mixture of 2-[3-butoxy-1-(2,4-dichlorobenzyl)-1H-pyrazol-5-yl]ethanesulfonamide (300 mg), 4-dimethylaminopyridine (181 mg), N,N-diisopropylethylamine (191 mg) and N,N-dimethylacetamide (5 ml) was added isopropyl chloroformate (181 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:24-3:7, v/v), concentrated, and crystallized from ethyl acetate-hexane to give isopropyl ({2-[3-butoxy-1-(2,4-dichlorobenzyl)-1H-pyrazol-5-yl]ethyl}sulfonyl)carbamate (240 mg, yield: 66%) as colorless crystals. melting point 120-121° C.

### Example 140

Under ice-cooling, to a mixture of 2-[3-butoxy-1-(2,4-dichlorobenzyl)-1H-pyrazol-5-yl]ethanesulfonamide (300 mg), 4-dimethylaminopyridine (181 mg), N,N-diisopropylethylamine (191 mg) and N,N-dimethylacetamide (5 ml) was added 2,2-dimethylpropyl chloroformate (223 mg), and the mixture was stirred at room temperature for 15 hr. The reaction mixture was concentrated, 1N hydrochloric acid was added to the residue, and the mixture was extracted with ethyl acetate. The extract was washed with water and saturated brine, dried (MgSO₄), and concentrated. The residue was subjected to silica gel column chromatography, and eluted with ethyl acetate-hexane (1:24-, 3:7, v/v), concentrated, and crystallized from ethyl acetate-hexane to give 2,2-dimethylpropyl ({2-[3-butoxy-1-(2,4-dichlorobenzyl)-1H-pyrazol-5-yl]ethyl}sulfonyl)carbamate (150 mg, yield: 39%) as colorless crystals. melting point 116-118° C.

### Example 141

Under ice-cooling, to a mixture of 3-methylbutane-1-sulfonamide (230 mg), pyridine (156 mg) and toluene (10 ml) was added triphosgene (226 mg), and the mixture was stirred at room temperature for 1 hr. The reaction mixture was immersed in an ice bath, [1-(2,4-dichlorobenzyl)-3-isopropoxy-1H-pyrazol-5-yl]methanol (400 mg), N,N-diisopropylethylamine (821 mg), 4-dimethylaminopyridine (155 mg) and tetrahydrofuran (10 ml) were added, and the mixture was stirred at room temperature for 15 hr. The reaction mixture was concentrated, 1N hydrochloric acid was added to the residue, and the mixture was extracted with ethyl acetate. The extract was washed with saturated brine, dried (MgSO₄), and concentrated. The residue was subjected to silica gel column chromatography, and eluted with ethyl acetate-hexane (1:19-1:3, v/v), concentrated, and crystallized from ethyl acetate-hexane to give [1-(2,4-dichlorobenzyl)-3-isopropoxy-1H-pyrazol-5-yl]methyl [(3-methylbutyl)sulfonyl]carbamate (210 mg, yield: 34%) as colorless crystals. melting point 104-107° C.

### Example 142

Under ice-cooling, to a mixture of pentane-1-sulfonamide (289 mg), pyridine (196 mg) and toluene (10 ml) was added triphosgene (283 mg), and the mixture was stirred at room temperature for 1 hr. The reaction mixture was immersed in an ice bath, [1-(2,4-dichlorobenzyl)-3-isopropoxy-1H-pyrazol-5-yl]methanol (500 mg), N,N-diisopropylethylamine (1.03 g), 4-dimethylaminopyridine (194 mg) and tetrahydrofuran (10 ml) were added, and the mixture was stirred at room temperature for 15 hr. The reaction mixture was concentrated, 1N hydrochloric acid was added to the residue, and the mixture was extracted with ethyl acetate. The extract was washed with saturated brine, dried (MgSO₄), and concentrated. The residue was subjected to silica gel column chromatography, and eluted with ethyl acetate-hexane (1:19-1:3, v/v), concentrated, and crystallized from ethyl acetate-hexane to give [1-(2,4-dichlorobenzyl)-3-isopropoxy-1H-pyrazol-5-yl]methyl (pentylsulfonyl)carbamate (150 mg, yield: 19%) as colorless crystals. melting point 126-128° C.

### Example 143

To a solution of 3-{1-[2-chloro-4-(trifluoromethyl)benzyl]-3-isopropoxy-1H-pyrazol-5-yl}propionic acid (502 mg) in tetrahydrofuran (10 ml) was added N,N′-carbonyldiimidazole (310 mg), and the mixture was heated under reflux for 1 hr. After cooling to room temperature, pentane-1-sulfonamide (201 mg) and 1,8-diazabicyclo[5.4.0]undec-7-ene (0.30 ml) were added to the reaction mixture, and the mixture was stirred for 7 hr. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:4-1:1, v/v) to give a white solid. Recrystallization from ethyl acetate-hexane gave 3-{1-[2-chloro-4-(trifluoromethyl)benzyl]-3-isopropoxy-1H-pyrazol-5-yl}-N-(pentylsulfonyl)propanamide (39 mg, yield: 6%) as white crystals. melting point 147.0-149.0° C.

### Example 144

To a solution of ethyl 3-[3-isopropoxy-1-(pyridin-2-ylmethyl)-1H-pyrazol-5-yl]propionate (1.09 g) in tetrahydrofuran (3.5 ml) and ethanol (3.5 ml) was added a 1N aqueous sodium hydroxide solution (7.5 ml), and the mixture was stirred at 50° C. for 1 hr. After cooling to room temperature, 1N hydrochloric acid (7.5 ml) was added, concentrated, and the residue was dissolved in ethyl acetate. The organic layer was washed twice with saturated brine, dried (MgSO₄), filtrated and concentrated to give a white solid.

To a solution of the obtained solid in tetrahydrofuran (8 ml) was added N,N′-carbonyldiimidazole (167 mg), and the mixture was heated under reflux for 1.5 hr. After cooling to room temperature, pentane-1-sulfonamide (115 mg) and 1,8-diazabicyclo[5.4.0]undec-7-ene (0.25 ml) were added to the reaction mixture, and the mixture was stirred for 4 hr. The reaction mixture was concentrated and the concentrate was dissolved in ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-4:1, v/v) to give 3-[3-isopropoxy-1-(pyridin-2-ylmethyl)-1H-pyrazol-5-yl]-N-(pentylsulfonyl)propanamide (45 mg, yield: 15%) as a pale-yellow oil.

¹H-NMR (300 MHz, CDCl₃) δ:0.84-0.94 (3H, m), 1.24 (6H, d, J=6.2 Hz), 1.27-1.45 (4H, m), 1.64-1.81 (2H, m), 2.66 (2H, t, J=7.1 Hz), 3.07 (2H, t, J=7.1 Hz), 3.16-3.27 (2H, m), 4.50-4.65 (1H, m), 5.16 (2H, s), 5.48 (1H, s), 7.30-7.38 (1H, m), 7.43 (1H, d, J=7.7 Hz), 7.75-7.85 (1H, m), 8.70 (1H, d, J=4.1 Hz).

### Example 145

To a solution of [3-butoxy-1-(2,4-dichlorobenzyl)-1H-pyrazol-5-yl]acetic acid (501 mg) in tetrahydrofuran (10 ml) was added N,N′-carbonyldiimidazole (322 mg), and the mixture was heated under reflux for 50 min. After cooling to room temperature, pentane-1-sulfonamide (215 mg) and 1,8-diazabicyclo[5.4.0]undec-7-ene (0.30 ml) were added to the reaction mixture, and the mixture was stirred overnight. The reaction mixture was concentrated and the concentrate was dissolved in ethyl acetate. The organic layer was washed with saturated brine, dried (MgSO₄), filtrated and concentrated. The obtained residue was subjected to silica gel column chromatography, and eluted with ethyl acetate-hexane (1:9-1:4, v/v) to give 2-[3-butoxy-1-(2,4-dichlorobenzyl)-1H-pyrazol-5-yl]-N-(pentylsulfonyl)acetamide (171 mg, yield: 25%) as a pale-orange solid.

¹H-NMR (300 MHz, CDCl₃) δ:0.81-1.08 (6H, m), 1.23-1.90 (10H, m), 3.25-3.36 (2H, m), 3.60 (2H, s), 4.11 (2H, t, J=6.6 Hz), 5.20 (2H, s), 5.74 (1H, s), 6.75 (1H, d, J=8.3 Hz), 7.19 (1H, dd, J=8.4, 2.0 Hz), 7.39 (1H, d, J=2.1 Hz)

### Example 146

To a solution of 4-[3-butoxy-1-(2,4-dichlorobenzyl)-1H-pyrazol-5-yl]butanoic acid (1.36 g) in tetrahydrofuran (15 ml) was added N,N′-carbonyldiimidazole (891 mg), and the mixture was heated under reflux for 1 hr 20 min. After cooling to room temperature, pentane-1-sulfonamide (645 mg) and 1,8-diazabicyclo[5.4.0]undec-7-ene (0.80 ml) were added to the reaction mixture, and the mixture was stirred for 6 hr. The reaction mixture was concentrated and the concentrate was dissolved in ethyl acetate. The organic layer was washed with 1N hydrochloric acid, a saturated aqueous sodium hydrogencarbonate solution, and saturated brine, dried (MgSO₄), filtrated and concentrated. The obtained residue was subjected to silica gel column chromatography, and eluted with ethyl acetate-hexane (1:9-1:1, v/v) to give a white solid. Recrystallization from ethyl acetate-hexane gave 4-[3-butoxy-1-(2,4-dichlorobenzyl)-1H-pyrazol-5-yl]-N-(pentylsulfonyl)butanamide (989 mg, yield: 54%) as white crystals. melting point 121.0-122.0° C.

### Example 147

To a solution of 5-[3-butoxy-1-(2,4-dichlorobenzyl)-1H-pyrazol-5-yl]pentanoic acid (433 mg) in tetrahydrofuran (10 ml) was added N,N′-carbonyldiimidazole (312 mg), and the mixture was heated under reflux for 30 min. After cooling to room temperature, pentane-1-sulfonamide (205 mg) and 1,8-diazabicyclo[5.4.0]undec-7-ene (0.25 ml) were added to the reaction mixture, and the mixture was stirred overnight. The reaction mixture was concentrated and the concentrate was dissolved in ethyl acetate. The organic layer was washed with saturated aqueous ammonium chloride solution, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give a white solid. Recrystallization from ethyl acetate-hexane gave 5-[3-butoxy-1-(2,4-dichlorobenzyl)-1H-pyrazol-5-yl]-N-(pentylsulfonyl)pentanamide (290 mg, yield: 50%) as white crystals. melting point 91-93° C.

### Example 148

A solution of 3-[1-(2,4-dichlorobenzyl)-3-isopropoxy-1H-pyrazol-5-yl]propanoic acid (0.50 g) and N,N′-carbonyldiimidazole (0.25 g) in tetrahydrofuran (10 ml) was stirred with heating under reflux for 1 hr. After cooling to room temperature, methanesulfonamide (0.15 g) and 1,8-diazabicyclo[5.4.0]undec-7-ene (0.24 ml) were added, and the mixture was stirred overnight at room temperature. 1N Hydrochloric acid (8 ml) and brine (10 ml) were added to the reaction mixture, and the mixture was extracted with ethyl acetate (20 ml×2). The organic layer was washed with brine, dried (MgSO₄), filtrated and concentrated. The residue was subjected to silica gel column chromatography, and eluted with ethyl acetate-hexane (3:1, v/v), concentrated, and the obtained crude crystals were recrystallized from ethyl acetate-hexane to give 3-[1-(2,4-dichlorobenzyl)-3-isopropoxy-1H-pyrazol-5-yl]-N-(methylsulfonyl)propanamide (0.53 g, yield: 86%) as colorless crystals. melting point 104-105° C.

### Example 149

A solution of (2E)-3-[1-[2-chloro-4-(trifluoromethyl)benzyl]-3-(2-methoxyethoxy)-1H-pyrazol-5-yl]acrylic acid (405 mg) and N,N′-carbonyldiimidazole (195 mg) in N,N-dimethylformamide (4 ml) was stirred at room temperature for 1 hr. To the mixture were added pentane-1-sulfonamide (181 mg) and 1,8-diazabicyclo[5.4.0]undec-7-ene (0.179 ml), and the mixture was stirred at 100° C. for 12 hr. After cooling to room temperature, 1N hydrochloric acid was added to the reaction mixture to adjust the pH to about 4. Water was added to the mixture, and the mixture was extracted with ethyl acetate. The organic layer was washed with saturated brine, dried (MgSO₄), filtrated and concentrated. The residue was subjected to silica gel column chromatography, and eluted with ethyl acetate-hexane (1:20-2:3, v/v), concentrated, and the obtained crude crystals were recrystallized from ethyl acetate-hexane to give (2E)-3-[1-[2-chloro-4-(trifluoromethyl)benzyl]-3-(2-methoxyethoxy)-1H-pyrazol-5-yl]-N-(pentylsulfonyl)acrylamide (278 mg, yield: 52%) as colorless crystals. melting point 130.5-132.5° C.

### Example 150

A solution of (2E)-3-{3-butoxy-1-[2-chloro-4-(trifluoromethyl)benzyl]-1H-pyrazol-5-yl}acrylic acid (500 mg) and N,N′-carbonyldiimidazole (250 mg) in N,N-dimethylformamide (5 ml) was stirred at room temperature for 1 hr. To the mixture were added 3-methylbutane-1-sulfonamide (233 mg) and 1,8-diazabicyclo[5.4.0]undec-7-ene (0.232 ml), and the mixture was stirred at 100° C. for 12 hr. After cooling to room temperature, 1N hydrochloric acid was added to the reaction mixture, and the mixture was adjusted to about pH 4. Water was added to the mixture, and the mixture was extracted with ethyl acetate. The organic layer was washed with saturated brine, dried (MgSO₄), filtrated and concentrated. The residue was subjected to silica gel column chromatography, and eluted with ethyl acetate-hexane (1:20-2:3, v/v), concentrated, and the obtained crude crystals were recrystallized from ethyl acetate-hexane to give (2E)-3-{3-butoxy-1-[2-chloro-4-(trifluoromethyl)benzyl]-1H-pyrazol-5-yl}-N-[(3-methylbutyl)sulfonyl]acrylamide (304 mg, yield: 57%) as colorless crystals. melting point 89.5-90.5° C.

### Example 151

A solution of (2E)-3-[1-[2-chloro-4-(trifluoromethyl)benzyl]-3-(cyclopropylmethoxy)-1H-pyrazol-5-yl]acrylic acid (500 mg) and N,N′-carbonyldiimidazole (250 mg) in N,N-dimethylformamide (5 ml) was stirred at room temperature for 1 hr. To the mixture were added pentane-1-sulfonamide (233 mg) and 1,8-diazabicyclo[5.4.0]undec-7-ene (0.232 ml), and the mixture was stirred at 100° C. for 12 hr. After cooling to room temperature, 1N hydrochloric acid was added to the reaction mixture, and the mixture was adjusted to about pH 4. Water was added to the mixture, and the mixture was extracted with ethyl acetate. The organic layer was washed with saturated brine, dried (MgSO₄), filtrated and concentrated. The residue was subjected to silica gel column chromatography, and eluted with ethyl acetate-hexane (1:20-2:3, v/v), concentrated, and the obtained crude crystals were recrystallized from ethyl acetate-hexane to give (2E)-3-[1-[2-chloro-4-(trifluoromethyl)benzyl]-3-(cyclopropylmethoxy)-1H-pyrazol-5-yl]-N-(pentylsulfonyl)acrylamide (345 mg, yield: 65%) as colorless crystals. melting point 148-151° C.

### Example 152

A solution of (2E)-3-[1-[2-chloro-4-(trifluoromethyl)benzyl]-3-(2-methoxyethoxy)-1H-pyrazol-5-yl]acrylic acid (500 mg) and N,N′-carbonyldiimidazole (240 mg) in N,N-dimethylformamide (5 ml) was stirred at room temperature for 1 hr. To the mixture were added 3-methylbutane-1-sulfonamide (224 mg) and 1,8-diazabicyclo[5.4.0]undec-7-ene (0.222 ml), and the mixture was stirred at 100° C. for 12 hr. After cooling to room temperature, 1N hydrochloric acid was added to the reaction mixture, and the mixture was adjusted to about pH 4. Water was added to the mixture, and the mixture was extracted with ethyl acetate. The organic layer was washed with saturated brine, dried (MgSO₄), filtrated and concentrated. The residue was subjected to silica gel column chromatography, and eluted with ethyl acetate-hexane (1:20-2:3, v/v), and concentrated. The obtained crude crystals were recrystallized from ethyl acetate-hexane. Diisopropyl ether was added to the obtained crude crystals, and the mixture was stirred for 1 hr, and recrystallized from methanol-water to give (2E)-3-[1-[2-chloro-4-(trifluoromethyl)benzyl]-3-(2-methoxyethoxy)-1H-pyrazol-5-yl]-N-[(3-methylbutyl)sulfonyl]acrylamide (292 mg, yield: 54%) as colorless crystals. melting point 114-116° C.

### Example 153

A solution of (2E)-3-[1-[2-chloro-4-(trifluoromethyl)benzyl]-3-(tetrahydro-2H-pyran-4-yloxy)-1H-pyrazol-5-yl]acrylic acid (535 mg) and N,N′-carbonyldiimidazole (221 mg) in N,N-dimethylformamide (5 ml) was stirred at room temperature for 1 hr. To the mixture were added 3-methylbutane-1-sulfonamide (206 mg) and 1,8-diazabicyclo[5.4.0]undec-7-ene (0.203 ml), and the mixture was stirred at 100° C. for 12 hr. After cooling to room temperature, 1N hydrochloric acid was added to the reaction mixture, and the mixture was adjusted to about pH 4. Water was added to the mixture, and the mixture was extracted with ethyl acetate. The organic layer was washed with saturated brine, dried (MgSO₄), filtrated and concentrated. The residue was subjected to silica gel column chromatography, eluted with ethyl acetate-hexane (1:10-1:2, v/v), concentrated, and the obtained crude crystals were recrystallized from ethyl acetate-hexane to give (2E)-3-[1-[2-chloro-4-(trifluoromethyl)benzyl]-3-(tetrahydro-2H-pyran-4-yloxy)-1H-pyrazol-5-yl]-N-[(3-methylbutyl)sulfonyl]acrylamide (315 mg, yield: 45%) as colorless crystals. melting point 149.5-152.5° C.

### Example 154

A solution of (2E)-3-[1-[2-chloro-4-(trifluoromethyl)benzyl]-3-(tetrahydro-2H-pyran-4-yloxy)-1H-pyrazol-5-yl]acrylic acid (535 mg) and N,N′-carbonyldiimidazole (221 mg) in N,N-dimethylformamide (5 ml) was stirred at room temperature for 1 hr. To the mixture were added pentane-1-sulfonamide (206 mg) and 1,8-diazabicyclo[5.4.0]undec-7-ene (0.203 ml), and the mixture was stirred at 100° C. for 12 hr. After cooling to room temperature, 1N hydrochloric acid was added to the reaction mixture, and the mixture was adjusted to about pH 4. Water was added to the mixture, and the mixture was extracted with ethyl acetate. The organic layer was washed with saturated brine, dried (MgSO₄), filtrated and concentrated. The residue was subjected to silica gel column chromatography, and eluted with ethyl acetate-hexane (1:10-1:2, v/v), concentrated, and the obtained crude crystals were recrystallized from ethyl acetate-hexane to give (2E)-3-[1-[2-chloro-4-(trifluoromethyl)benzyl]-3-(tetrahydro-2H-pyran-4-yloxy)-1H-pyrazol-5-yl]-N-(pentylsulfonyl)acrylamide (337 mg, yield: 48%) as colorless crystals. melting point 160.5-161° C.

### Example 155

A solution of (2E)-3-{1-[2-chloro-4-(trifluoromethyl)benzyl]-3-cyclopropyl-1H-pyrazol-5-yl}acrylic acid (501 mg) and N,N′-carbonyldiimidazole (242 mg) in N,N-dimethylformamide (5 ml). was stirred at room temperature for 1 hr. To the mixture were added pentane-1-sulfonamide (225 mg) and 1,8-diazabicyclo[5.4.0]undec-7-ene (0.223 ml), and the mixture was stirred at 100° C. for 12 hr. After cooling to room temperature, 1N hydrochloric acid was added to the reaction mixture, and the mixture was adjusted to about pH 4. Water was added to the mixture, and the mixture was extracted with ethyl acetate. The organic layer was washed with saturated brine, dried-(MgSO₄), filtrated and concentrated. The residue was subjected to silica gel column chromatography, eluted with ethyl acetate-hexane (1:20-1:2, v/v), concentrated, and the obtained crude crystals were recrystallized from ethyl acetate-hexane to give (2E)-3-{1-[2-chloro-4-(trifluoromethyl)benzyl]-3-cyclopropyl-1H-pyrazol-5-yl}-N-(pentylsulfonyl)acrylamide (313 mg, yield: 46%) as colorless crystals. melting point 139-140.5° C.

### Example 156

A solution of 3-{1-[2-chloro-4-(trifluoromethyl)benzyl]-3-cyclopropyl-1H-pyrazol-5-yl}propanoic acid (450 mg) and N,N′-carbonyldiimidazole (216 mg) in N,N-dimethylformamide (5 ml) was stirred at room temperature for 1 hr. To the mixture were added pentane-1-sulfonamide (201 mg) and 1,8-diazabicyclo[5.4.0]undec-7-ene (0.199 ml), and the mixture was stirred at 100° C. for 12 hr. After cooling to room temperature, 1N hydrochloric acid was added to the reaction mixture, and the mixture was adjusted to about pH 4. Water was added to the mixture, and the mixture was extracted with ethyl acetate. The organic layer was washed with saturated brine, dried (MgSO₄), filtrated and concentrated. The residue was subjected to silica gel column chromatography, eluted with ethylacetate-hexane (1:20-1:2, v/v), and concentrated. The obtained crude crystals were recrystallized from ethyl acetate-hexane. The obtained crude crystals were purified by HPLC (acetonitrile:water, containing 0.01% TFA, 40:60-100:0, v/v), and recrystallized from ethyl acetate-hexane to give 3-{1-[2-chloro-4-(trifluoromethyl)benzyl]-3-cyclopropyl-1H-pyrazol-5-yl}-N-(pentylsulfonyl)propanamide (261 mg, yield: 43%) as colorless crystals. melting point 151.5-153.5° C.

### Example 157

A solution of 3-{1-[2-chloro-4-(trifluoromethyl)benzyl]-3-cyclopropyl-1H-pyrazol-5-yl}propanoic acid (450 mg) and N,N′-carbonyldiimidazole (216 mg) in N,N-dimethylformamide (5 ml) was stirred at room temperature for 1 hr. 3-Methylbutane-1-sulfonamide (201 mg) and 1,8-diazabicyclo[5.4.0]undec-7-ene (0.199 ml) were added, and the mixture was stirred at 100° C. for 12 hr. After cooling to room temperature, 1N hydrochloric acid was added to the reaction mixture, and the mixture was adjusted to about pH 4. Water was added to the mixture, and the mixture was extracted with ethyl acetate. The organic layer was washed with saturated brine, dried (MgSO₄), filtrated and concentrated. The residue was subjected to silica gel column chromatography, eluted with ethyl acetate-hexane (1:20-1:2, v/v), and concentrated. The obtained crude crystals were recrystallized from ethyl acetate-hexane. The obtained crude crystals were purified by HPLC (acetonitrile:water, containing 0.01% TFA, 40:60-100:0, v/v), and recrystallized from ethyl acetate-hexane to give 3-{1-[2-chloro-4-(trifluoromethyl)benzyl]-3-cyclopropyl-1H-pyrazol-5-yl}-N-[(3-methylbutyl)sulfonyl]propanamide (191 mg, yield: 31%) as colorless crystals. melting point 160.5-162.5° C.

### Example 158

Toga solution (10 ml) of (2E)-3-[2-(cyclohexylmethoxy)-4-(2-methoxyethoxy)phenyl]acrylic acid (0.86 g) in acetonitrile, 1-ethyl-3-(3-dimethylaminopropyl)carbodiimide hydrochloride (0.74 g), 4-dimethylaminopyridine (0.47 g) and pentane-1-sulfonamide (0.43 g) were added, and the mixture was stirred at room temperature for 15 hr. The reaction mixture was concentrated, water was poured into the obtained residue, and the mixture was extracted with ethyl acetate. The ethyl acetate layer was washed with saturated brine, dried (MgSO₄), and concentrated. The obtained residue was subjected to silica gel column chromatography, and eluted with and eluted with ethyl acetate-hexane (1:1, v/v). The obtained crude crystals were recrystallized from ethyl acetate-hexane to give (2E)-3-[2-(cyclohexylmethoxy)-4-(2-methoxyethoxy)phenyl]-N-(pentylsulfonyl)acrylamide (0.30 g, yield: 25%) as colorless crystals. melting point 97.5-98.3° C.

### Example 159

A solution of (2E)-3-[2-(cyclohexylmethoxy)-4-(2-methoxyethoxy)phenyl]-N-(pentylsulfonyl)acrylamide (165 mg) and 10% palladium-carbon (40 mg) in methanol (20 ml) was stirred under a hydrogen atmosphere at room temperature for 2 hr. The catalyst was filtered off, and the filtrate was concentrated. The obtained residue was subjected to silica gel column chromatography, and eluted with ethyl acetate-hexane (3:7, v/v). The obtained crude crystals were recrystallized from ethyl acetate-hexane to give 3-[2-(cyclohexylmethoxy)-4-(2-methoxyethoxy)phenyl]-N-(pentylsulfonyl)propanamide (151 mg, yield: 91%) as colorless crystals. melting point 81.0-82.0° C.

### Example 160

To absolution of (2E)-3-[2-(cyclopropylmethoxy)-4-(2-methoxyethoxy)phenyl]acrylic acid (400 mg) in tetrahydrofuran (8 ml) was added N,N′-carbonyldiimidazole (338 mg), and the mixture was heated under reflux for 30 min. After cooling to room temperature, 1,8-diazabicyclo[5.4.0]-7-undecene (310 mg) and pentane-1-sulfonamide (215 mg) were added to the reaction mixture, and the mixture was stirred at 60° C. for 100 hr. Water was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v) to give (2E)-3-[2-(cyclopropylmethoxy)-4-(2-methoxyethoxy)phenyl]-N-(pentylsulfonyl)acrylamide (147 mg, yield: 25%) as colorless crystals. melting point 64.8-66.3° C.

### Example 161

To a mixed solution of (2E)-3-[2-{[1-(tert-butoxycarbonyl)piperidin-4-yl]oxy}-4-(2-methoxyethoxy)phenyl]acrylic acid (558 mg) in acetonitrile (8 ml) and N,N-dimethylformamide (5 ml) were added 1-ethyl-3-(3-dimethylaminopropyl)carbodiimide hydrochloride (334 mg), 4-dimethylaminopyridine (246 mg) and pentane-1-sulfonamide (232 mg), and the mixture was stirred at room temperature for 5 hr. Water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (3:2, v/v) to give tert-butyl 4-(5-(2-methoxyethoxy)-2-{(1E)-3-oxo-3-[(pentylsulfonyl)amino]prop-1-en-1-yl}phenoxy)piperidine-1-carboxylate (177 mg, yield: 24%) as a colorless amorphous solid.

¹H-NMR (300 MHz, CDCl₃) δ:0.90 (3H, t, J=7.2 Hz), 1.25-1.47 (4H, m), 1.47 (9H, s), 1.83-1.86 (4H, m), 1.92-2.00 (2H, m), 3.26-3.32 (2H, m), 3.45 (3H, s), 3.46-3.54 (2H, m), 3.74-3.79 (4H, m), 4.13-4.16 (2H, m), 4.47-4.53 (1H, m), 6.43 (11H, d, J=15.6 Hz), 6.51-6.52 (2H, m), 7.41-7.44 (1H, m), 7.95 (1H, d, J=15.6 Hz), 8.12 (1H, brs).

### Example 162

A solution of tert-butyl 4-(5-(2-methoxyethoxy)-2-{(1E)-3-oxo-3-[(pentylsulfonyl)amino]prop-1-en-1-yl}phenoxy)piperidine-1-carboxylate (181 mg) and 10% palladium-carbon (40 mg) in methanol (20 ml) was stirred under a hydrogen atmosphere at room temperature for 2 hr. The catalyst was filtered off, and the filtrate was concentrated. The obtained residue was subjected to silica gel column chromatography, and eluted with ethyl acetate-hexane (1:2, v/v) to give tert-butyl 4-(5-(2-methoxyethoxy)-2-{3-oxo-3-[(pentylsulfonyl)amino]propyl}phenoxy)piperidine-1-carboxylate (147 mg) as a colorless amorphous solid. To a solution of the obtained colorless amorphous solid in ethyl acetate (4 ml) was added a 4N hydrogen chloride ethyl acetate solution (2 ml), and the mixture was stirred at room temperature for 2 hr. The reaction mixture was concentrated and dried to give (2E)-3-[4-(2-methoxyethoxy)-2-(piperidin-4-yloxy)phenyl]-N-(pentylsulfonyl)acrylamide (123 mg, yield: 94%) as a colorless amorphous solid.

¹H-NMR (300 MHz, DMSO-d₆) δ:0.84 (3H, t, J=7.1 Hz), 1.23-1.31 (4H, m), 1.50-1.60 (2H, m), 1.79-1.90 (2H, m), 2.02-2.13 (2H, m), 2.48-2.50 (2H, m), 2.70-2.75 (2H, m), 3.10-3.18 (4H, m), 3.27-3.29 (2H, m), 3.29 (3H, s), 3.62-3.64 (2H, m), 4.02-4.05 (2H, m), 4.65-4.70 (1H, m), 6.45 (1H, d, J=8.2 Hz), 6.61 (1H, s), 7.02 (1H, d, J=8.2 Hz), 8.73 (2H, brs), 11.61 (1H, brs).

### Example 163

To a solution of (2E)-3-[2-(cyclohexyloxy)-4-(2-methoxyethoxy)phenyl]acrylic acid (517 mg) in tetrahydrofuran (10 ml) was added N,N″-carbonyldiimidazole (392 mg), and the mixture was heated under reflux for 1 hr. After cooling to room temperature, 1,8-diazabicyclo[5.4.0]-7-undecene (386 mg), 4-dimethylaminopyridine (238 mg) and pentane-1-sulfonamide (374 mg) were added to the reaction mixture, and the mixture was heated under reflux for 22 hr. Water was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v) to give (2E)-3-[2-(cyclohexyloxy)-4-(2-methoxyethoxy)phenyl]-N-(pentylsulfonyl)acrylamide (321 mg, yield: 44%) as a colorless amorphous solid.

¹H-NMR (300 MHz, CDCl₃) δ:0.90 (3H, t J=7.1 Hz), 1.31-1.45 (6H, m), 1.52-1.65 (4H, m), 1.78-1.90 (4H, m), 1.97-2.05 (2H, m), 3.46 (3H, s), 3.48-3.55 (2H, m), 3.76-3.77 (2H, m), 4.13-4.16 (2H, m), 4.26-4.35 (1H, m), 6.45-6.53 (3H, m), 7.40 (1H, d, J=8.4 Hz), 7.70 (1H, brs), 7.94 (1H, d, J=15.6 Hz).

### Example 164

A solution of (2E)-3-[2-(cyclohexyloxy)-4-(2-methoxyethoxy)phenyl]-N-(pentylsulfonyl)acrylamide (238 mg) and 10% palladium-carbon (40 mg) in methanol (20 ml) was stirred under a hydrogen atmosphere at room temperature for 3 hr. The catalyst was filtered off, and the filtrate was concentrated. The obtained residue was subjected to silica gel column chromatography, and eluted with ethyl acetate-hexane (3:7, v/v) to give 3-[2-(cyclohexyloxy)-4-(2-methoxyethoxy)phenyl]-N-(pentylsulfonyl)propanamide (246 mg, yield: 99%) as colorless crystals. melting point 64.1-65.4° C.

### Example 165

To a solution of (2E)-3-[4-(2-methoxyethoxy)-2-(tetrahydrofuran-2-ylmethoxy)phenyl]acrylic acid (500 mg) in acetonitrile (5 ml) were added 2-methyl-6-nitrobenzoic anhydride (533 mg), triethylamine (495 mg), 4-dimethylaminopyridine (189 mg) and pentane-1-sulfonamide (239 mg), and the mixture was stirred at room temperature for 28 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v) to give (2E)-3-[4-(2-methoxyethoxy)-2-(tetrahydrofuran-2-ylmethoxy)phenyl]-N-(pentylsulfonyl)acrylamide (489 mg, yield: 69%) as a colorless amorphous solid.

¹H-NMR (300 MHz, CDCl₃) δ:0.90 (3H, t, J=7.1 Hz), 1.26-1.48 (4H, m), 1.71-2.20 (6H, m), 3.45 (3H, s), 3.45-3.54 (2H, m), 3.74-3.77 (2H, m), 3.85-4.02 (4H, m), 4.11-4.16 (2H, m), 4.35-4.42 (1H, m), 6.52-6.54 (2H, m), 6.61 (1H, d, J=15.6 Hz), 7.38 (1H, d, J=8.1 Hz), 7.85 (1H, d, J=15.6 Hz), 8.56 (1H, brs).

### Example 166

A solution of (2E)-3-[4-(2-methoxyethoxy)-2-(tetrahydrofuran-2-ylmethoxy)phenyl]-N-(pentylsulfonyl)acrylamide (319 mg) and 10% palladium-carbon (40 mg) in methanol (20 ml) was stirred under a hydrogen atmosphere at room temperature for 2 hr. The catalyst was filtered off, and the filtrate was concentrated. The obtained residue was subjected to silica gel column chromatography, and eluted with ethyl acetate-hexane (2:3, v/v). The obtained crude crystals were recrystallized from ethyl acetate-hexane to give 3-[4-(2-methoxyethoxy)-2-(tetrahydrofuran-2-ylmethoxy)phenyl]-N-(pentylsulfonyl)propanamide (296 mg, yield: 92%) as colorless crystals. melting point 57.8-58.9° C.

### Example 167

A solution of (2E)-3-[2-(cyclopropylmethoxy)-4-(2-methoxyethoxy)phenyl]-N-(pentylsulfonyl)acrylamide (268 mg) and 10% palladium-carbon (41 mg) in methanol (20 ml) was stirred under a hydrogen atmosphere at room temperature for 2 hr. The catalyst was filtered off, and the filtrate was concentrated. The obtained residue was subjected to silica gel column chromatography, and eluted with ethyl acetate-hexane (3:7, v/v). The obtained crude crystals were recrystallized from ethyl acetate-hexane to give 3-[2-(cyclopropylmethoxy)-4-(2-methoxyethoxy)phenyl]-N-(pentylsulfonyl)propanamide (230 mg, yield: 85%) as colorless crystals. melting point 81.7-82.8° C.

### Example 168

To a solution of (2E)-3-[2-[(2,4-dichlorobenzyl)oxy]-4-(2-methoxyethoxy)phenyl]acrylic acid (530 mg) in acetonitrile (10 ml) were added 2-methyl-6-nitrobenzoic anhydride (461 mg), triethylamine (410 mg), 4-dimethylaminopyridine (166 mg) and pentane-1-sulfonamide (203 mg), and the mixture was stirred at room temperature for 40 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v). The obtained crude crystals were recrystallized from ethyl acetate-hexane to give (2E)-3-[2-[(2,4-dichlorobenzyl)oxy]-4-(2-methoxyethoxy)phenyl]-N-(pentylsulfonyl)acrylamide (385 mg, yield: 54%) as colorless crystals. melting point 137.6-138.2° C.

### Example 169

To a mixed solution of (2E)-3-[2-[(2,4-dichlorobenzyl)oxy]-4-(2-methoxyethoxy)phenyl]-N-(pentylsulfonyl)acrylamide (210 mg) in methanol (15 ml) and tetrahydrofuran (15 ml) was added a palladium-activated carbon ethylenediamine complex (30 mg), and the mixture was stirred under a hydrogen atmosphere at room temperature for 2 hr. The catalyst was filtered off, and the filtrate was concentrated. The obtained residue was subjected to silica gel column chromatography, and eluted with ethyl acetate-hexane (1:3, v/v). The obtained crude crystals were recrystallized from ethyl acetate-hexane to give 3-[2-[(2,4-dichlorobenzyl)oxy]-4-(2-methoxyethoxy)phenyl]-N-(pentylsulfonyl)propanamide (128 mg, yield: 60%) as colorless crystals. melting point 106.7-108.2° C.

### Example 170

To a solution of (2E)-3-[2-[(3,5-dichloropyridin-2-yl)oxy]-4-(2-methoxyethoxy)phenyl]acrylic acid (600 mg) in N,N-dimethylformamide (6 ml) were added 1-ethyl-3-(3-dimethylaminopropyl)carbodiimide hydrochloride (407 mg), 4-dimethylaminopyridine (290 mg) and pentane-1-sulfonamide (241 mg), and the mixture was stirred at room temperature for 72 hr. Water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:1, v/v). The obtained crude crystals were recrystallized from ethyl acetate-hexane to give (2E)-3-[2-[(3,5-dichloropyridin-2-yl)oxy]-4-(2-methoxyethoxy)phenyl]-N-(pentylsulfonyl)acrylamide (352 mg, yield: 43%) as colorless crystals. melting point 119.0-120.3° C.

### Example 171

To a mixed solution of (2E)-3-[2-[(3,5-dichloropyridin-2-yl) oxy]-4-(2-methoxyethoxy)phenyl]7-N-(pentylsulfonyl)acrylamide (167 mg) in methanol (10 ml) and tetrahydrofuran (10 ml) was added palladium-activated carbon ethylenediamine complex (20 mg), and the mixture was stirred under a hydrogen atmosphere at room temperature for 7 hr. The catalyst was filtered off, and the filtrate was concentrated. The obtained residue was subjected to silica gel column chromatography, and eluted with ethyl acetate-hexane (1:4, v/v). The obtained crude crystals were recrystallized from ethyl acetate-hexane to give 3-[2-[(3,5-dichloropyridin-2-yl) oxy]-4-(2-methoxyethoxy)phenyl]-N-(pentylsulfonyl)propanamide (116 mg, yield: 69%) as colorless crystals. melting point 92.2-93.6° C.

### Example 172

To a solution of (2E)-3-{4-(2-methoxyethoxy)-2-[(5-methyl-2-phenyl-1,3-oxazol-4-yl)methoxy]phenyl}acrylic acid (650 mg) in acetonitrile (10 ml) were added 2-methyl-6-nitrobenzoic anhydride (543 mg), triethylamine (489 mg), 4-dimethylaminopyridine (195 mg) and pentane-1-sulfonamide (242 mg), and the mixture was stirred at room temperature for 24 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v) to give (2E)-3-{4-(2-methoxyethoxy)-2-[(5-methyl-2-phenyl-1,3-oxazol-4-yl)methoxy]phenyl}-N-(pentylsulfonyl)acrylamide (780 mg, yield: 91%) as an amorphous solid.

¹H-NMR (300 MHz, CDCl₃) δ:0.87 (3H, t, J=7.1 Hz), 1.27-1.40 (4H, m), 1.75-1.83 (2H, m), 2.43 (3H, s), 3.40-3.44 (2H, m), 3.45 (3H, s), 3.74-3.78 (2H, m), 4.16-4.19 (2H, m), 5.03 (2H, s), 6.53-6.58 (2H, m), 6.76 (1H, d, J=2.1 Hz), 7.38-7.48 (4H, m), 7.85-7.91 (2H, m), 7.99-8.02 (2H, m).

### Example 173

A solution of (2E)-3-{4-(2-methoxyethoxy)-2-[(5-methyl-2-phenyl-1,3-oxazol-4-yl)methoxy]phenyl}-N-(pentylsulfonyl)acrylamide (270 mg) and 10% palladium-carbon (40 mg) in methanol (30 ml) was stirred under a hydrogen atmosphere at room temperature for 4 hr. The catalyst was filtered off, and the filtrate was concentrated. The obtained residue was subjected to silica gel column chromatography, and eluted with ethyl acetate-hexane (1:2, v/v). The obtained crude crystals were recrystallized from ethyl acetate-hexane to give 3-{4-(2-methoxyethoxy)-2-[(5-methyl-2-phenyl-1,3-oxazol-4-yl)methoxy]phenyl}-N-(pentylsulfonyl)propanamide (240 mg, yield: 88%) as colorless crystals. melting point 95.9-96.7° C.

### Example 174

To a solution of (2E)-3-{4-(2-methoxyethoxy)-2-[2-nitro-5-(trifluoromethyl)phenoxy]phenyl}acrylic acid (443 mg) in acetonitrile (8 ml) were added 1-ethyl-3-(3-dimethylaminopropyl)carbodiimide hydrochloride (309 mg), 4-dimethylaminopyridine (164 mg) and pentane-1-sulfonamide (157 mg), and the mixture was stirred at room temperature for 14 hr. Water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:3, v/v). The obtained crude crystals were recrystallized from ethyl acetate-hexane to give (2E)-3-{4-(2-methoxyethoxy)-2-[2-nitro-5-(trifluoromethyl)phenoxy]phenyl}-N-(pentylsulfonyl)acrylamide (75 mg, yield: 13%) as colorless crystals. melting point 164.5-165.2° C.

### Example 175

To absolution of (2E)-3-[2-[2-chloro-4-(trifluoromethyl)phenoxy]-4-(2-methoxyethoxy)phenyl]acrylic acid (715 mg) in acetonitrile (10 ml) were added 1-ethyl-3-(3-dimethylaminopropyl)carbodiimide hydrochloride (502 mg), 4-dimethylaminopyridine (278 mg) and pentane-1-sulfonamide (261 mg), and the mixture was stirred at room temperature for 18 hr. Water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:1, v/v). The obtained crude crystals were recrystallized from ethyl acetate-hexane to give (2E)-3-[2-[2-chloro-4-(trifluoromethyl)phenoxy]-4-(2-methoxyethoxy)phenyl]-N-(pentylsulfonyl)acrylamide (285 mg, yield: 30%) as colorless crystals. melting point 134.1-135.1° C.

### Example 176

To a solution of (2E)-3-(4-(2-methoxyethoxy)-2-{[4-(trifluoromethyl)phenyl]amino}phenyl)acrylic acid (350 mg) in acetonitrile (6 ml) were added 2-methyl-6-nitrobenzoic anhydride (350 mg), triethylamine (283 mg), 4-dimethylaminopyridine (135 mg) and pentane-1-sulfonamide (144 mg), and the mixture was stirred at room temperature for 30 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v). The obtained crude crystals were recrystallized from ethyl acetate-hexane to give (2E)-3-(4-(2-methoxyethoxy)-2-{[4-(trifluoromethyl)phenyl]amino}phenyl)-N-(pentylsulfonyl)acrylamide (149 mg, yield: 31%) as yellow crystals. melting point 142.3-142.6° C.

### Example 177

A solution of (2E)-3-(4-(2-methoxyethoxy)-2-{[4-(trifluoromethyl)phenyl]amino}phenyl)-N-(pentylsulfonyl)acrylamide (200 mg) and 10% palladium-carbon (35 mg) in methanol (20 ml) was stirred under a hydrogen atmosphere at room temperature for 2 hr. The catalyst was filtered off, and the filtrate was concentrated. The obtained residue was subjected to silica gel column chromatography, and eluted with ethyl acetate-hexane (1:2, v/v) to give 3-(4-(2-methoxyethoxy)-2-{[4-(trifluoromethyl)phenyl]amino}phenyl)-N(pentylsulfonyl)propanamide (101 mg, yield: 50%) as an amorphous solid.

¹H-NMR (300 MHz, CDCl₃) δ:0.86 (3H, t, J=6.9 Hz), 1.22-1.38 (4H, m), 1.62-1.71 (2H, m), 2.65 (2H, t, J=6.5 Hz), 2.88 (2H, t, J=6.5 Hz), 3.29-3.34 (2H, m), 3.43 (3H, s), 3.71-3.73 (2H, m), 4.03-4.06 (2H, m), 6.58 (1H, s), 6.70 (1H, dd, J=8.4, 2.4 Hz), 6.86 (1H, d, J=2.4 Hz), 6.92 (2H, d, J=8.7 Hz), 7.11 (1H, d, J=8.4 Hz), 7.45 (2H, d, J=8.7 Hz), 7.84 (1H, s).

### Example 178

To a solution of (2E)-3-[2-[(5-bromo-1,3-thiazol-2-yl)oxy]-4-(2-methoxyethoxy)phenyl]acrylic acid (660 mg) in acetonitrile (10 ml) were added 2-methyl-6-nitrobenzoic anhydride (573 mg), triethylamine (497 mg), 4-dimethylaminopyridine (206 mg) and pentane-1-sulfonamide (249 mg), and the mixture was stirred at room temperature for 20 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (2:3, v/v). The obtained crude crystals were recrystallized from ethyl acetate-hexane to give (2E)-3-[2-[(5-bromo-1,3-thiazol-2-yl)methyl]-4-(2-methoxyethoxy)phenyl]-N-(pentylsulfonyl)acrylamide (676 mg, yield: 77%) as yellow crystals. melting point 88.5-89.1° C.

### Example 179

To a solution of tert-butyl 4-(5-(2-methoxyethoxy)-2-{(1E)-3-oxo-3-((pentylsulfonyl)amino]prop-1-en-1-yl}phenoxy)piperidine-1-carboxylate (1.02 g) in ethyl acetate (12 ml), a 4N hydrogen chloride ethyl acetate solution (6 ml) was added, and the mixture was stirred at room temperature for 90 min. The reaction mixture was concentrated and dried to give (2E)-3-[4-(2-methoxyethoxy)-2-(piperidin-4-yloxy)phenyl]-N-(pentylsulfonyl)acrylamide hydrochloride (859 mg, yield: 95%) as a pale-yellow amorphous solid.

¹H-NMR (300 MHz, DMSO-d₆) δ:0.85 (3H, t, J=7.1 Hz), 1.22-1.40 (4H, m), 1.60-1.72 (2H, m), 1.80-1.95 (2H, m), 2.10-2.18 (2H, m), 3.02-3.30 (4H, m), 3.30 (3H, s), 3.34-3.48 (2H, m), 3.64-3.66 (2H, m), 4.14-4.17 (2H, m), 4.77-4.82 (1H, m), 6.57 (1H, d, J=15.6 Hz), 6.66 (1H, d, J=8.7 Hz), 6.75 (1H, s), 7.49 (1H, d, J=8.7 Hz), 7.79 (1H, d, J=15.6 Hz), 8.62 (1H, brs), 8.83 (1H, brs), 11.76 (1H, s).

### Example 180

To a solution of (2E)-3-[4-(2-methoxyethoxy)-2-(piperidin-4-yloxy)phenyl]-N-(pentylsulfonyl)acrylamide hydrochloride (175 mg) in pyridine (5 ml) were added acetic anhydride (110 mg) and 4-dimethylaminopyridine (45 mg), and the mixture was stirred at room temperature for 16 hr. The reaction mixture was concentrated, a saturated aqueous ammonium chloride solution was added to the obtained residue, and the mixture was extracted with ethyl acetate. The ethyl acetate layer was washed with saturated brine, dried (MgSO₄), and concentrated. The obtained residue was subjected to silica gel column chromatography, and eluted with ethyl acetate-methanol (19:1, v/v) to give (2E)-3-[2-[(1-acetylpiperidin-4-yl) oxy]-4-(2-methoxyethoxy)phenyl]-N-(pentylsulfonyl)acrylamide (110 mg, yield: 62%) as an amorphous solid.

¹H-NMR (300 MHz, CDCl₃) δ:0.90 (3H, t, J=7.1 Hz), 1.25-1.45 (4H, m), 1.80-2.06 (6H, m), 2.13 (3H, s), 3.39-3.55 (7H, m), 3.70-3.74 (1H, m), 3.74-3.77 (2H, m), 3.85-3.95 (1H, m), 4.13-4.16 (2H, m), 4.59-4.63 (1H, m), 6.51-6.56 (3H, m), 7.42 (1H, d, J=9.3 Hz), 7.91 (1H, d, J=15.6 Hz), 8.90 (1H, brs)

### Example 181

To a solution of (2E)-3-[4-(2-methoxyethoxy)-2-(piperidin-4-yloxy)phenyl]-N-(pentylsulfonyl)acrylamide hydrochloride (190 mg) in pyridine (5 ml) were added benzoyl chloride (91 mg) and triethylamine (83 mg), and the mixture was stirred at room temperature for 20 hr. The reaction mixture was concentrated, a saturated aqueous ammonium chloride solution was added to the obtained residue, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (4:1, v/v) to give (2E)-3-[2-[(1-benzoylpiperidin-4-yl)oxy]-4-(2-methoxyethoxy)phenyl]-N-(pentylsulfonyl)acrylamide (163 mg, yield: 75%) as a colorless amorphous solid.

¹H-NMR (300 MHz, CDCl₃) δ:0.89 (3H, t, J=7.1 Hz), 1.25-1.46 (4H, m), 1.78-2.20 (6H, m), 3.30-3.55 (6H, m), 3.55-3.90 (4H, m), 3.93-4.18 (3H, m), 4.61-4.65 (1H, m), 6.45-6.53 (3H, m), 7.38-7.43 (6H, m), 7.96 (1H, d, J=15.9 Hz), 8.54 (1H, s).

### Example 182

To a solution of (2E)-3-[2-[(5-bromo-1,3-thiazol-2-yl)methyl]-4-(2-methoxyethoxy)phenyl]-N-(pentylsulfonyl)acrylamide (260 mg) in 1,2-dimethoxyethane (6 ml) were added dihydroxyphenylborane (84 mg), a 2N sodium carbonate aqueous solution (0.5 ml) and tetrakis(triphenylphosphine)palladium (0) (56 mg), and the mixture was heated under reflux for 7 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:1, v/v) to give (2E)-3-{4-(2-methoxyethoxy)-2-[(5-phenyl-1,3-thiazol-2-yl)methyl]phenyl}-N-(pentylsulfonyl)acrylamide (135 mg, yield: 52%) as a pale-yellow amorphous solid.

¹H-NMR (300 MHz, CDCl₃) δ:0.87 (3H, t, J=7.1 Hz), 1.25-1.39 (4H, m), 1.78-1.88 (2H, m), 3.43 (3H, s), 3.43-3.49 (2H, m), 3.73-3.76 (2H, m), 4.13-4.16 (2H, m), 6.49 (1H, d, J=15.9 Hz), 6.86-6.92 (2H, m), 7.31-7.44 (6H, m), 7.51 (1H, d, J=9.0 Hz) 7.86 (1H, d, J=15.9 Hz), 8.59 (1H, s).

### Example 183

To a solution of 2-{[3-chloro-5-(trifluoromethyl)pyridin-2-yl]oxy}-4-(2-methoxyethoxy)benzaldehyde (1.40 g) in acetic acid (7 ml) were added ethylmalonic acid (2.46 g) and pyrrolidine (3.73 g), and the mixture was stirred at −100° C. for 72 hr. 1N Hydrochloric acid (1.5 ml) and water (14 ml) were added to the reaction mixture, and the mixture was stirred at room temperature for 1 hr. Water was poured into the obtained mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (3:7, v/v) to give (2E)-2-[2-{[3-chloro-5-(trifluoromethyl)pyridin-2-yl]oxy}-4-(2-methoxyethoxy)benzylidene]butanoic acid (450 mg) as a brown oil. To a solution of the obtained oil in acetonitrile (8 ml) were added 2-methyl-6-nitrobenzoic anhydride (276 mg), triethylamine (275 mg), 4-dimethylaminopyridine (101 mg) and pentane-1-sulfonamide (126 mg), and the mixture was stirred at room temperature for 18 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:3, v/v). The obtained crude crystals were recrystallized from ethyl acetate-hexane to give (2E)-2-[2-{[3-chloro-5-(trifluoromethyl)pyridin-2-yl]oxy}-4-(2-methoxyethoxy)benzylidene]-N-(pentylsulfonyl)butanamide (63 mg, yield: 3%) as colorless crystals. melting point 104.5-105.6° C.

### Example 184

To a solution of (2E)-3-[2-{[2-chloro-4-(trifluoromethyl)phenyl]amino}-4-(2-methoxyethoxy)phenyl]acrylic acid (487 mg) in acetonitrile (8 ml) were added 2-methyl-6-nitrobenzoic anhydride (449 mg), triethylamine (370 mg), 4-dimethylaminopyridine (144 mg) and pentane-1-sulfonamide (177 mg), and the mixture was stirred at room temperature for 22 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v). The obtained crude crystals were recrystallized from ethyl acetate-hexane to give (2E)-3-[2-{[2-chloro-4-(trifluoromethyl)phenyl]amino}-4-(2-methoxyethoxy)phenyl]-N-(pentylsulfonyl)acrylamide (255 mg, yield: 39%) as yellow crystals. melting point 173.5-174.8° C.

### Example 185

To a mixed solution of ethyl (2E)-3-{4-(2-methoxyethoxy)-2-[(3-methyl-5-nitropyridin-2-yl)oxy]phenyl}acrylate (630 mg) in tetrahydrofuran (8 ml) and ethanol (8 ml) was added a 1N aqueous sodium hydroxide solution (3.5 ml), and the mixture was stirred at 60° C. for 1 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained residue was recrystallized from ethanol-hexane to give (2E)-3-{4-(2-methoxyethoxy)-2-[(3-methyl-5-nitropyridin-2-yl)oxy]phenyl}acrylic acid (383 mg) as pale-yellow crystals. The obtained crystal were dissolved in acetonitrile (10 ml), and 2-methyl-6-nitrobenzoic anhydride (411 mg), triethylamine (305 mg), 4-dimethylaminopyridine (126 mg) and pentane-1-sulfonamide (156 mg) were added, and the mixture was stirred at room temperature for 24 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v). The obtained crude crystals were recrystallized from ethyl acetate-hexane to give (2E)-3-{4-(2-methoxyethoxy)-2-[(3-methyl-5-nitropyridin-2-yl)oxy]phenyl}-N-(pentylsulfonyl)acrylamide (178 mg, yield: 35%) as colorless crystals. melting point 148.0-150.3° C.

### Example 186

To a mixed solution of ethyl (2E)-3-(4-(2-methoxyethoxy)-2-{[6-(trifluoromethyl)pyridazin-3-yl]oxy}phenyl)acrylate (483 mg) in tetrahydrofuran (12 ml) and ethanol (8 ml) was added a 1N aqueous sodium hydroxide solution (2.5 ml), and the mixture was stirred at room temperature for 5 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated to give (2E)-3-(4-(2-methoxyethoxy)-2-{[6-(trifluoromethyl)pyridazin-3-yl]oxy}phenyl)acrylic acid (353 mg) as colorless crystals. The obtained crystals were dissolved in acetonitrile (10 ml), 2-methyl-6-nitrobenzoic anhydride (377 mg), triethylamine (280 mg), 4-dimethylaminopyridine (113 mg) and pentane-1-sulfonamide (139 mg) were added, and the mixture was stirred at room temperature for 24 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (2:3, v/v) to give (2E)-3-(4-(2-methoxyethoxy)-2-{[6-(trifluoromethyl)pyridazin-3-yl]oxy}phenyl)-N-(pentylsulfonyl)acrylamide (120 mg, yield: 20%) as a colorless amorphous solid.

¹H-NMR (300 MHz, CDCl₃) δ:0.87 (3H, t, J=7.1 Hz), 1.25-1.42 (4H, m), 1.75-1.84 (2H, m), 3.37-3.42 (2H, m), 3.43 (3H, s), 3.72-3.75 (2H, m), 4.12-4.15 (2H, m), 6.32 (1H, d, J=15.6 Hz), 6.77 (1H, d, J=2.4 Hz), 6.89 (1H, dd, J=9.0, 2.4 Hz), 7.45 (1H, d, J=9.0 Hz), 7.54 (1H, d, J=9.0 Hz), 7.76 (1H, d, J=15.6 Hz), 7.88 (1H, d, J=9.0 Hz), 8.32 (1H, s).

### Example 187

To a solution of (2E)-3-[2-[(3,5-dichloropyridin-2-yl)oxy]-4-(2-methoxyethoxy)phenyl]-2-methylacrylic acid (358 mg) in acetonitrile (8 ml) were added 2-methyl-6-nitrobenzoic anhydride (324 mg), triethylamine (275 mg), 4-dimethylaminopyridine (113 mg) and pentane-1-sulfonamide (138 mg), and the mixture was stirred at room temperature for 20 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:2, v/v). The obtained crude crystals were recrystallized from ethyl acetate-hexane to give (2E)-3-[2-[(3,5-dichloropyridin-2-yl)oxy]-4-(2-methoxyethoxy)phenyl]-2-methyl-N-(pentylsulfonyl)acrylamide (421 mg, yield: 88%) as yellow crystals. melting point 97.0-98.0° C.

### Example 188

To a solution of (2E)-2-[2-[(3,5-dichloropyridin-2-yl)oxy]-4-(2-methoxyethoxy)benzylidene]butanoic acid (325 mg) in acetonitrile (8 ml) were added 2-methyl-6-nitrobenzoic anhydride (332 mg), triethylamine (247 mg), 4-dimethylaminopyridine (98 mg) and pentane-1-sulfonamide (130 mg), and the mixture was stirred at room temperature for 44 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (3:7, v/v). The obtained crude crystals were recrystallized from ethyl acetate-hexane to give (2E)-2-[2-[(3,5-dichloropyridin-2-yl)oxy]-4-(2-methoxyethoxy)benzylidene]-N-(pentylsulfonyl)butanamide (363 mg, yield: 84%) as colorless crystals. melting point 118.0-118.4° C.

### Example 189

To a solution of (2E)-3-[2-[(2,4-dichlorobenzoyl)amino]-4-(2-methoxyethoxy)phenyl]acrylic acid (304 mg) in acetonitrile (8 ml) were added 2-methyl-6-nitrobenzoic anhydride (359 mg), triethylamine (235 mg), 4-dimethylaminopyridine (99 mg) and pentane-1-sulfonamide (136 mg), and the mixture was stirred at room temperature for 20 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v). The obtained crude crystals were recrystallized from ethyl acetate-hexane to give 2,4-dichloro-N-(5-(2-methoxyethoxy)-2-{(1E)-3-oxo-3-[(pentylsulfonyl)amino]prop-1-en-1-yl}phenyl)benzamide (160 mg, yield: 40%) as colorless crystals. melting point 176.8-177.1° C.

### Example 190

To a solution of (2E)-3-[2-[(3,5-dichloropyridin-2-yl)oxy]-4-(3-methoxypropoxy)phenyl]-2-methylacrylic acid (338 mg) in acetonitrile (8 ml) were added 2-methyl-6-nitrobenzoic anhydride (312 mg), triethylamine (265 mg), 4-dimethylaminopyridine (100 mg) and pentane-1-sulfonamide (130 mg), and the mixture was stirred at room temperature for 20 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:3, v/v). The obtained crude crystals were recrystallized from ethyl acetate-hexane to give (2E)-3-[2-[(3,5-dichloropyridin-2-yl)oxy]-4-(3-methoxypropoxy)phenyl]-2-methyl-N-(pentylsulfonyl)acrylamide (394 mg, yield: 84%) as yellow crystals. melting point 116.0-116.8° C.

### Example 191

To a solution of (2E)-3-[2-[(2,4-difluorobenzoyl)amino]-4-(2-methoxyethoxy)phenyl]acrylic acid (366 mg) in acetonitrile (10 ml) were added 2-methyl-6-nitrobenzoic anhydride (369 mg), triethylamine (340 mg), 4-dimethylaminopyridine (120 mg) and pentane-1-sulfonamide (157 mg), and the mixture was stirred at room temperature for 24 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v). The obtained crude crystals were recrystallized from ethyl acetate-hexane to give 2,4-difluoro-N-(5-(2-methoxyethoxy)-2-{(1E)-3-oxo-3-[(pentylsulfonyl)amino]prop-1-en-1-yl}phenyl)benzamide (158 mg, yield: 32%) as colorless crystals. melting point 140.0-140.8° C.

### Example 192

To a solution of (2E)-3-[2-({5-[(tert-butoxycarbonyl)amino]-3-methylpyridin-2-yl}oxy)-4-(2-methoxyethoxy)phenyl]acrylic acid (256 mg) in acetonitrile (8 ml) were added 2-methyl-6-nitrobenzoic anhydride (244 mg), triethylamine (180 mg), 4-dimethylaminopyridine (76 mg) and pentane-1-sulfonamide (99 mg), and the mixture was stirred at room temperature for 22 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v). The obtained crude crystals were recrystallized from ethyl acetate-hexane to give tert-butyl[6-(5-(2-methoxyethoxy)-2-{(1E)-3-oxo-3-[(pentylsulfonyl)amino]prop-1-en-1-yl}phenoxy)-5-methylpyridin-3-yl]carbamate (288 mg, yield: 86%) as yellow crystals. melting point 149.5-150.2° C.

### Example 193

To a solution of (2E)-3-(2-{[3-chloro-5-(trifluoromethyl)pyridin-2-yl]oxy}-4-{2-[(triisopropylsilyl)oxy]ethoxy}phenyl)acrylic acid (7.90 g). in acetonitrile (30 ml) were added 1-ethyl-3-(3-dimethylaminopropyl)carbodiimide hydrochloride (3.51 g), 4-dimethylaminopyridine (2.59 g) and 1-pentanesulfonamide (2.19 g), and the mixture was stirred at room temperature for 16 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:20-1:4, v/v) to give (2E)-3-(2-{[3-chloro-5-(trifluoromethyl)pyridin-2-yl]oxy}-4-{2-[(triisopropylsilyl)oxy]ethoxy}phenyl)-N-(pentylsulfonyl)acrylamide (4.50 g, yield: 46%) as a white solid. Recrystallization from diisopropyl ether-hexane gave a white powder. melting point 106-108° C.

¹H-NMR (300 MHz, CDCl₃) δ:0.89 (3H, t, J=7.2 Hz), 0.98-1.17 (21H, m), 1.21-1.49 (4H, m), 1.70-1.92 (2H, m), 3.35-3.56 (2H, m), 3.91-4.19 (4H, m), 6.37 (1H, d, J=15.8 Hz), 6.70 (1H, d, J=2.5 Hz), 6.90 (1H, dd, J=8.7, 2.5 Hz), 7.60 (1H, d, J=8.9 Hz), 7.72-7.83 (2H, m), 8.03 (1H, d, J=2.1 Hz), 8.25 (1H, dd, J=2.0, 0.9 Hz).

### Example 194

To a solution of (2E)-3-[2-{[3-chloro-5-(trifluoromethyl)pyridin-2-yl]oxy}-4-(2-hydroxyethoxy)phenyl]-N-(pentylsulfonyl)acrylamide (252 mg) in pyridine (2 ml) was added acetic anhydride (0.088 ml) under ice-cooling, and the mixture was stirred for 1 hr. A saturated aqueous sodium hydrogencarbonate solution was added to the reaction mixture, and the mixture was extracted with ethyl acetate. The organic layer was washed with water and saturated brine, dried, and concentrated. The obtained residue was subjected to silica gel column chromatography, and eluted with ethyl acetate-hexane (1:1, v/v). The obtained crude crystals were recrystallized from ethyl acetate-hexane to give 2-(3-{[3-chloro-5-(trifluoromethyl)pyridin-2-yl]oxy}-4-{(1E)-3-oxo-3-[(pentylsulfonyl)amino]prop-1-en-1-yl}phenoxy)ethyl acetate (237 mg, yield: 87%) as white crystals. melting point 141-144° C.

### Example 195

To a solution of (2E)-3-[2-{[3-chloro-5-(trifluoromethyl)pyridin-2-yl]oxy}-4-(tetrahydrofuran-2-ylmethoxy)phenyl]acrylic acid (0.79 g) in acetonitrile (6 ml) were added 1-ethyl-3-(3-dimethylaminopropyl)carbodiimide hydrochloride (541 mg), 4-dimethylaminopyridine (398 mg) and pentane-1-sulfonamide (269 mg), and the mixture was stirred at room temperature for 16 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:20-1:4, v/v) to give a white solid. Recrystallization from diethyl ether-hexane gave (2E)-3-[2-{[3-chloro-5-(trifluoromethyl)pyridin-2-yl]oxy}-4-(tetrahydrofuran-2-ylmethoxy)phenyl]-N-(pentylsulfonyl)acrylamide (202 mg, yield: 20%) as a white powder. melting point 126.5-128.5° C.

### Example 196

To a solution of (2E)-3-{2-{[3-chloro-5-(trifluoromethyl)pyridin-2-yl]oxy}-4-[2-(1,3-dioxolan-2-yl)ethoxy]phenyl}acrylic acid (1.00 g) in; acetonitrile (6 ml) were added 1-ethyl-3-(3-dimethylaminopropyl)carbodiimide hydrochloride (541 mg), 4-dimethylaminopyridine (398 mg) and pentane-1-sulfonamide (328 mg), and the mixture was stirred at room temperature for 16 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:20-1:4, v/v) to give a white solid. Recrystallization from ethyl acetate-hexane gave (2E)-3-{2-{[3-chloro-5-(trifluoromethyl)pyridin-2-yl]oxy}-4-[2-(1,3-dioxolan-2-yl)ethoxy]phenyl}-N-(pentylsulfonyl)acrylamide (194 mg, yield: 15%) as a white powder. melting point 116-118° C.

### Example 197

To a solution of (2E)-3-[2-{[3-chloro-5-(trifluoromethyl)pyridin-2-yl]oxy}-4-(pyrimidin-2-yloxy)phenyl]acrylic acid (1.00 g) in acetonitrile (12 ml) were added 1-ethyl-3-(3-dimethylaminopropyl)carbodiimide hydrochloride (782 mg), 4-dimethylaminopyridine (374 mg) and pentane-1-sulfonamide (295 mg), and the mixture was stirred at room temperature for 16 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:20-1:2, v/v) to give a white solid. Recrystallization from diethyl ether-hexane gave (2E)-3-[2-{[3-chloro-5-(trifluoromethyl)pyridin-2-yl]oxy}-4-(pyrimidin-2-yloxy)phenyl]-N-(pentylsulfonyl)acrylamide (140 mg, yield: 12%) as a white powder. melting point 80.5-82.5° C.

### Example 198

To a solution of (2E)-3-{2-{[3-chloro-5-(trifluoromethyl)pyridin-2-yl]oxy}-4-[2-ethoxy-1-(ethoxymethyl)ethoxy]phenyl}acrylic acid (831 mg) in acetonitrile (12 ml) were added 1-ethyl-3-(3-dimethylaminopropyl)carbodiimide hydrochloride (652 mg), 4-dimethylaminopyridine (312 mg) and pentane-1-sulfonamide (257 mg), and the mixture was stirred at room temperature for 16 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:20-1:4, v/v) to give a white solid. Recrystallization from diisopropyl ether-hexane gave (2E)-3-{2-{[3-chloro-5-(trifluoromethyl)pyridin-2-yl]oxy}-4-[2-ethoxy-1-(ethoxymethyl)ethoxy]phenyl}-N-(pentylsulfonyl)acrylamide (397 mg, yield: 38%) as a white powder. melting point 83-85.5° C.

¹H-NMR (300 MHz, CDCl₃) δ:1.26-1.44 (7H, m), 1.46-1.70 (4H, m), 1.70-2.04 (4H, m), 3.45 (3H, s), 3.73-3.76 (2H, m), 4.11-4.14 (2H, m), 4.20-4.32 (3H, m), 6.38-6.52 (3H, m), 7.42 (1H, d, J=8.7 Hz), 7.93 (1H, d, J=16.2 Hz).

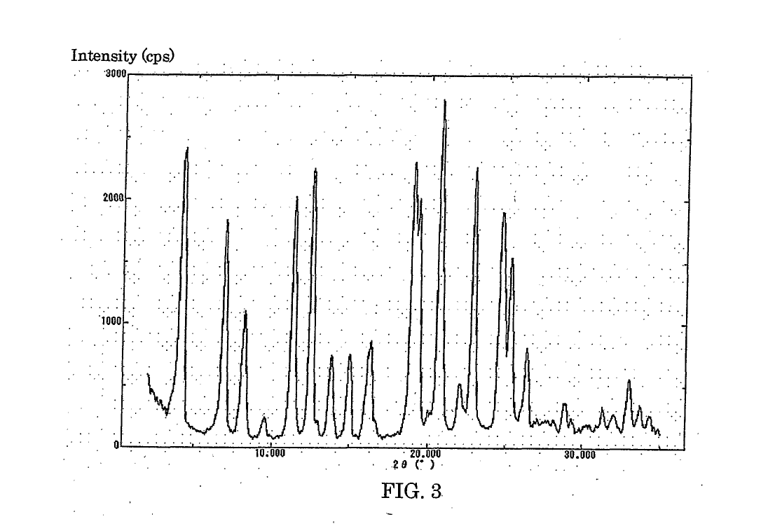

Recrystallization of the crude crystals obtained under the same conditions as in Example 198 from diethyl ether-hexane gave crystals. The X-ray powder diffraction pattern of this crystal as measured using Cu-1 ray (tube voltage: 40 KV; tube current: 50 mA) as a radiation source and RINT2100 type Ultima+(Rigaku Corporation) is shown in FIG. 3.

Data (main peak) of X-ray powder diffraction

| diffraction angle: 2θ (°) | spacing: d value (angstrom) |
|---|---|
| 4.20 | 21.0 |
| 6.92 | 12.8 |
| 11.3 | 7.80 |
| 12.5 | 7.06 |
| 19.0 | 4.68 |
| 20.7 | 4.28 |
| 22.9 | 3.88 |
| 24.7 | 3.60 |

### Example 199

A mixture of (2E)-3-(1-{[3-chloro-5-(trifluoromethyl)pyridin-2-yl]methyl}-3-isopropoxy-1H-pyrazol-5-yl)acrylic acid (220 mg), N,N′-carbonyldiimidazole (137 mg) and N′,N-dimethylformamide (6.0 ml) was stirred at room temperature for 1 hr, pentane-1-sulfonamide (102 mg) and 1,8-diazabicyclo[5.4.0]undec-7-ene (129 mg) were added, and the mixture was stirred at 100° C. for 4 hr. After cooling to room temperature, the reaction mixture was poured into 1N hydrochloric acid, and the mixture was extracted with ethyl acetate. The extract was washed with water and saturated brine, dried (MgSO₄), and concentrated. The residue was subjected to silica gel column chromatography, and eluted with ethyl acetate-hexane (1:19-1:2, v/v) to give (2E)-3-(1-{[3-chloro-5-(trifluoromethyl)pyridin-2-yl]methyl}-3-isopropoxy-1H-pyrazol-5-yl)-N-(pentylsulfonyl)acrylamide (30 mg, yield: 10%) as a pale-yellow oil.

¹H-NMR (300 MHz, CDCl₃) δ:0.89 (3H, t, J=7.2 Hz), 1.22-1.49 (10H, m), 1.75-1.90 (2H, m), 3.40-3.51 (2H, m), 4.61-4.74 (1H, m), 5.58 (2H, s), 6.07 (1H, s), 6.34 (1H, d, J=15.3 Hz), 7.62 (1H, d, J=15.3 Hz), 7.91-7.96 (1H, m), 8.61-8.65 (1H, m), 8.74 (1H, m)

### Example 200

Under ice-cooling, to a solution of tert-butyl {[(diphenylphosphoryl)methyl]sulfonyl}carbamate (9.03 g) in N,N-dimethylformamide (150 ml) was added sodium hydride (60% in oil, 2.28 g), and the mixture was stirred under an argon atmosphere at room temperature for 30 min. The reaction mixture was ice-cooled again, a solution of 1-(2,4-dichlorobenzyl)-3-(methoxymethoxy)-1H-pyrazole-5-carbaldehyde (4.80 g) in N,N-dimethylformamide (40 ml) was added, and the mixture was stirred under an argon atmosphere at 0° C. for 1 hr. The reaction mixture was poured into a saturated sodium chloride aqueous solution, and the mixture was extracted with ethyl acetate. The extract was washed with water and saturated brine, dried (MgSO₄), and concentrated. The residue was subjected to silica gel column chromatography, eluted with ethyl acetate-hexane (1:9-2:1, v/v), concentrated, and crystallized from ethyl acetate-hexane to give tert-butyl ({(E)-2-[1-(2,4-dichlorobenzyl)-3-(methoxymethoxy)-1H-pyrazol-5-yl]vinyl}sulfonyl)carbamate (3.16 g, yield: 42%) as colorless crystals. melting point 139-140° C.

### Example 201

To a solution of (2E)-3-[2-{[3-chloro-5-(trifluoromethyl)pyridin-2-yl]oxy}-4-(2-methoxyethoxy)phenyl]acrylic acid (1.00 g) in tetrahydrofuran (25 ml) was added N,N′-carbonyldiimidazole (604 mg), and the mixture was heated under reflux for 1 hr. After allowing to cool to room temperature, 3-methoxypropane-1-sulfonamide (0.57 g) and 1,8-diazabicyclo[5.4.0]undec-7-ene (0.70 ml) were added to the reaction mixture, and the mixture was stirred overnight. The reaction mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (15:85-9:1, v/v) to give (2E)-3-[2-{[3-chloro-5-(trifluoromethyl)pyridin-2-yl]oxy}-4-(2-methoxyethoxy)phenyl]-N-[(3-methoxypropyl)sulfonyl]acrylamide (138 mg, yield: 10%) as a white amorphous form.

¹H-NMR (300 MHz, CDCl₃) δ:2.01-2.16 (2H, m), 3.30 (3H, s), 3.44 (3H, s), 3.47 (2H, t, J=5.9 Hz), 3.54-3.64 (2H, m), 3.70-3.82 (2H, m), 4.06-4.20 (2H, m), 6.38 (1H, d, J=15.6 Hz), 6.68-6.76 (1H, m), 6.91 (1H, dd, J=8.8, 2.5 Hz), 7.60 (1H, d, J=8.9 Hz), 7.79 (1H, d, J=15.6 Hz), 8.03 (1H, d, J=2.1 Hz), 8.25 (1H, dd, J=2.1, 0.9 Hz).

### Example 202

To a solution of 3-(2-{[3-chloro-5-(trifluoromethyl)pyridin-2-yl]oxy}-4-isopropoxyphenyl)propanoic acid (0.50 g) in tetrahydrofuran (15 ml) was added N,N′-carbonyldiimidazole (340 mg), and the mixture was heated under reflux for 1 hr. After allowing to cool to room temperature, 3-methoxypropane-1-sulfonamide (0.31 g) and 1,8-diazabicyclo[5.4.0]undec-7-ene (0.40 ml) were added to the reaction mixture, and the mixture was stirred overnight. The reaction. mixture was concentrated and the concentrate was dissolved in ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-1:1, v/v) to give a white solid. Recrystallization from ethyl acetate-hexane gave 3-(2-{[3-chloro-5-(trifluoromethyl)pyridin-2-yl]oxy}-4-isopropoxyphenyl)-N-[(3-methoxypropyl)sulfonyl]propanamide (4.12 g, yield: 99%) as white crystals. melting point 114-115° C.

### Example 203

To a solution of 3-[2-{[3-chloro-5-(trifluoromethyl)pyridin-2-yl]oxy}-4-(2-methoxyethoxy)phenyl]propan-1-ol (499 mg) in dichloromethane (5 ml) was added chlorosulfonylisocyanate (0.15 ml) under ice-cooling, and the mixture was stirred for 30 min. Pyridine (0.30 ml) was added to the reaction mixture and the mixture was stirred for 1 hr. 3-Methyl-1-butylamine (0.40 ml) was added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give a white solid. Recrystallization from ethyl acetate-hexane then from ethanol-water gave 3-[2-{[3-chloro-5-(trifluoromethyl)pyridin-2-yl]oxy}-4-(2-methoxyethoxy)phenyl]propyl {[(3-methylbutyl)amino]sulfonyl}carbamate (85 mg, yield: 12%) as white crystals. melting point 123.0-123.5° C.

¹H-NMR (300 MHz, CDCl₃) δ:1.14 (6H, d, J=6.0 Hz), 1.84-2.00 (2H, m), 2.56 (2H, t, J=7.4 Hz), 3.15-3.27 (2H, m), 3.44 (3H, s), 3.53 (2H, t, J=5.2 Hz), 3.55-3.62 (1H, m), 3.73 (2H, dd, J=5.5, 4.0 Hz), 4.09 (2H, dd, J=5.5, 4.0 Hz), 4.14 (2H, t, J=6.3 Hz), 5.40 (1H, t, J=5.8 Hz), 6.68 (1H, d, J=2.6 Hz), 6.83 (1H, dd, J=8.5, 2.6 Hz), 7.19 (1H, d, J=8.7 Hz), 7.47 (1H, s), 8.01 (1H, d, J=2.1 Hz), 8.27 (1H, dd, J=−2.1, 0.9 Hz).

### Example 204

To a solution of 3-[2-{[3-chloro-5-(trifluoromethyl)pyridin-2-yl]oxy}-4-(2-methoxyethoxy)phenyl]propan-1-ol (497 mg) in dichloromethane (5 ml) was added chlorosulfonylisocyanate (0.15 ml) under ice-cooling, and the mixture was stirred for 30 min. Pyridine (0.30 ml) was added to the reaction mixture, and the mixture was stirred for 1 hr. 2-Isopropoxyethylamine (0.40 ml) was added, and the mixture was stirred overnight at room temperature. 1N Hydrochloric acid was added to the reaction mixture, and the mixture was diluted with ethyl acetate. The organic layer was washed with a saturated aqueous sodium hydrogencarbonate solution and saturated brine, dried (MgSO₄), filtrated and concentrated to give a white solid. Recrystallization from ethyl acetate-hexane gave 3-[2-{[3-chloro-5-(trifluoromethyl)pyridin-2-yl]oxy}-4-(2-methoxyethoxy)phenyl]propyl {[(2-isopropoxyethyl)amino]sulfonyl}carbamate (730 mg, yield: 97%) as white crystals. melting point 113.5-114.0° C.

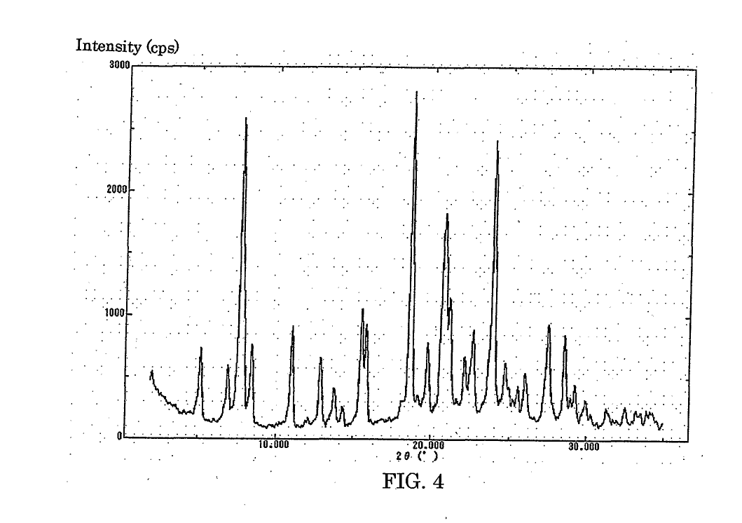

Recrystallization of the crude crystals obtained under the same conditions as in Example 204 from ethanol-hexane gave crystals. The X-ray powder diffraction pattern of this crystal as measured using Cu-Kα ray (tube voltage: 40 KV; tube current: 50 mA) as a radiation source and RINT2100 type Ultima+(Rigaku Corporation) is shown in FIG. 4.

Data (main peak) of X-ray powder diffraction

| diffraction angle: 2θ (°) | spacing: d value (angstrom) |
|---|---|
| 7.74 | 11.4 |
| 11.0 | 8.01 |
| 15.5 | 5.70 |
| 18.7 | 4.75 |
| 20.8 | 4.27 |
| 23.9 | 3.72 |
| 27.5 | 3.24 |

### Example 205

To a solution of 3-[2-{[3-chloro-5-(trifluoromethyl)pyridin-2-yl]oxy}-4-(2-methoxyethoxy)phenyl]propan-1-ol (495 mg) in N,N-dimethylformamide (5 ml) was added N,N′-carbonyldiimidazole (296 mg), and the mixture was stirred at 40° C. for 1 hr. After allowing to cool to room temperature, N-(4-methylcyclohexyl)sulfamide (254 mg) and 1,8-diazabicyclo[5.4.0]undec-7-ene (0.30 ml) were added to the reaction mixture, and the mixture was stirred overnight. 1N Hydrochloric acid was added to the reaction mixture, and the mixture was extracted with ethyl acetate. The organic layer was washed with water and saturated brine, dried (MgSO₄), filtrated and concentrated to give a pale-yellow solid. The obtained residue was subjected to silica gel column chromatography, and eluted with ethyl acetate-hexane (1:19-2:3, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]propyl {[(4-methylcyclohexyl)amino]sulfonyl}carbamate (323 mg, yield: 43%) as white crystals. melting point 121.5-121.8° C.

### Example 206

To a solution of 3-[2-{[3-chloro-5-(trifluoromethyl)pyridin-2-yl]oxy}-4-(2-methoxyethoxy)phenyl]propan-1-ol (504 mg) in dichloromethane (5 ml) was added chlorosulfonylisocyanate (0.15 ml) under ice-cooling, and the mixture was stirred for 30 min. Pyridine (0.30 ml) was added to the reaction mixture, and the mixture was stirred for 1 hr. 2-Phenylethylamine (0.40 ml) was added, and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]propyl {[(2-phenylethyl)amino]sulfonyl}carbamate (596 mg, yield: 76%) as white crystals. melting point 110-113° C.

### Example 207

To a solution of 3-[2-{[3-chloro-5-(trifluoromethyl)pyridin-2-yl]oxy}-4-(2-methoxyethoxy)phenyl]propan-1-ol (493 mg) in N,N-dimethylformamide (5 ml) was added N,N′-carbonyldiimidazole (291 mg), and the mixture was stirred at 40° C. for 1 hr. After allowing to cool to room temperature, N-[2-(2-thienyl)ethyl]sulfamide (275 mg) and 1,8-diazabicyclo[5.4.0]undec-7-ene (0.30 ml) were added to the reaction mixture, and the mixture was stirred overnight. 1N Hydrochloric acid was added to the reaction mixture, and the mixture was diluted with ethyl acetate. The organic layer was washed with water and saturated brine, dried (MgSO₄), filtrated and concentrated to give a pale-yellow solid. The obtained residue was subjected to silica gel column chromatography, and eluted with ethyl acetate-hexane (1:19-45:55, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]propyl ({[2-(2-thienyl)ethyl]amino}sulfonyl)carbamate (319 mg, yield: 41%) as white crystals. melting point 117.5-118.0° C.

### Example 208

To a solution of 3-[2-{[3-chloro-5-(trifluoromethyl)pyridin-2-yl]oxy}-4-(2-methoxyethoxy)phenyl]propan-1-ol (483 mg) in dichloromethane (5 ml) was added chlorosulfonylisocyanate (0.15 ml) under ice-cooling, and the mixture was stirred for 30 min. Pyridine (0.30 ml) was added to the reaction mixture, and the mixture was stirred for 1 hr. 2-Pyridin-2-ylethylamine (0.40 g) was added, and the mixture was stirred overnight at room temperature. A saturated aqueous ammonium chloride solution was added to the reaction mixture, and the mixture was diluted with ethyl acetate. The organic layer was washed with a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-9:1, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]propyl {[(2-pyridin-2-ylethyl)amino]sulfonyl}carbamate (439 mg, yield: 58%) as a white powder. melting point 91-93° C.

¹H-NMR (300 MHz, CDCl₃) δ:1.78-1.97 (2H, m), 2.53 (2H, t, J=7.4 Hz), 3.06 (2H, t, J=6.2 Hz), 3.43 (3H, s), 3.52 (2H, t, J=6.2 Hz), 3.73 (2H, dd, J=5.5, 4.0 Hz), 3.98-4.15 (4H, m), 6.37 (1H, br. s.), 6.67 (1H, d, J=2.4 Hz), 6.81 (1H, dd, J=8.5, 2.6 Hz), 7.09-7.21 (3H, m), 7.61 (1H, ddd, J=7.6, 7.6, 1.9 Hz), 7.99 (1H, d, J=1.9 Hz), 8.26 (1H, dd, J 2.1, 0.9 Hz), 8.49 (1H, dd, J=5.1, 1.9 Hz).

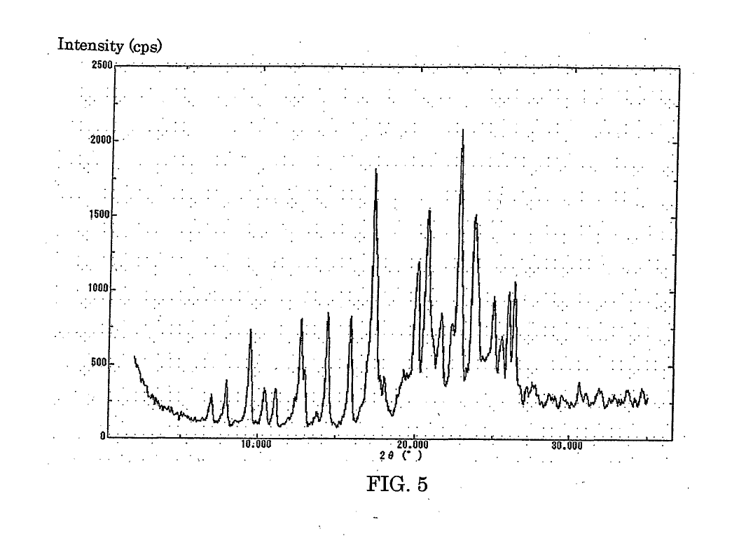

Recrystallization of the crude crystals obtained under the same conditions as in Example 208 from ethyl acetate-hexane gave crystals. The X-ray powder diffraction pattern of this crystal as measured using Cu-Kα ray (tube voltage: 40 KV; tube current: 50 m) as a radiation source and RINT2100 type Ultima+ (Rigaku Corporation) is shown in FIG. 5.

Data (main peak) of X-ray powder diffraction

| diffraction angle: 2θ (°) | spacing: d value (angstrom) |
|---|---|
| 9.44 | 9.36 |
| 12.7 | 6.94 |
| 14.4 | 6.13 |
| 15.9 | 5.56 |
| 17.3 | 5.12 |
| 20.1 | 4.42 |
| 20.7 | 4.30 |
| 22.7 | 3.91 |
| 23.7 | 3.75 |

### Example 209

To a solution of 3-[2-{[3-chloro-5-(trifluoromethyl)pyridin-2-yl]oxy}-4-(2-methoxyethoxy)phenyl]-2-methylpropanoic acid (495 mg) in dichloromethane (10 ml) were added 1-ethyl-3-(3-dimethylaminopropyl)carbodiimide (242 mg), 4-dimethylaminopyridine (252 mg) and pentane-1-sulfonamide (243 mg), and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]-2-methyl-N-(pentylsulfonyl)propanamide as white crystals. melting point 136.5-138.0° C.

### Example 210

To absolution of 3-[2-{[3-chloro-5-(trifluoromethyl)pyridin-2-yl]oxy}-4-(2-methoxyethoxy)phenyl]-2-methoxypropanoic acid (512 mg) in dichloromethane (10 ml) were added 1-ethyl-3-(3-dimethylaminopropyl)carbodiimide (269 mg), 4-dimethylaminopyridine (278 mg) and pentane-1-sulfonamide (266 mg), and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-65:35, v/v) to give a white solid. Recrystallization from ethyl acetate-hexane gave white crystals. The obtained crystals were subjected to silica gel column chromatography, and eluted with ethyl acetate-hexane (1:9-1:2, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]-2-methoxy-N-(pentylsulfonyl)propanamide (154 mg, yield: 23%) as white crystals. melting point 76.0-78.0° C.

### Example 211

To a solution of (2E)-3-[2-{[3-chloro-5-(trifluoromethyl)pyridin-2-yl]oxy}-4-(2-methoxyethoxy)phenyl]-2-methylacrylic acid (220 mg) in dichloromethane (10 ml) were added 1-ethyl-3-(3-dimethylaminopropyl)carbodiimide (201 mg), 4-dimethylaminopyridine (299 mg) and pentane-1-sulfonamide (106 mg), and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v) to give a white solid. Recrystallization from ethyl acetate-hexane gave (2E)-3-[2-{[3-chloro-5-(trifluoromethyl)pyridin-2-yl]oxy}-4-(2-methoxyethoxy)phenyl]-2-methyl-N-(pentylsulfonyl)acrylamide (158 mg, yield: 55%) as white crystals. melting point 108-111° C.

### Example 212

To a solution of N-(aminosulfonyl)-3-[2-{[3-chloro-5-(trifluoromethyl)pyridin-2-yl]oxy}-4-(2-methoxyethoxy)phenyl]propanamide (201 mg) in tetrahydrofuran (20 ml) were added triphenylphosphine (143 mg), 1-butanol (0.10 ml) and a diethyl azodicarboxylate 40%-toluene solution (0.30 ml) under ice-cooling, and the mixture was stirred overnight at room temperature. The reaction mixture was concentrated, and the obtained residue was subjected to silica gel column chromatography and eluted with ethyl acetate-hexane (hexane alone to 1:1, v/v) to give a white solid. This was subjected to basic silica gel column chromatography, and eluted with ethyl acetate-hexane (hexane alone to ethyl acetate alone) to give a white solid. Recrystallization from ethyl acetate-hexane gave N-(aminosulfonyl)-N-butyl-3-[2-{[3-chloro-5-(trifluoromethyl)pyridin-2-yl]oxy}-4-(2-methoxyethoxy)phenyl]propanamide (30 mg, yield: 13%) as white crystals. melting point 102-103° C.

### Example 213

To a solution of N-(aminosulfonyl)-3-[2-{[3-chloro-5-(trifluoromethyl)pyridin-2-yl]oxy}-4-(2-methoxyethoxy)phenyl]propanamide (201 mg) in tetrahydrofuran (20 ml) were added triphenylphosphine (143 mg), 2-methoxyethanol (0.10 ml) and a diethyl azodicarboxylate 40%-toluene solution (0.30 ml) under ice-cooling, and the mixture was stirred overnight at room temperature. The reaction mixture was concentrated, and the obtained residue was subjected to silica gel column chromatography and eluted with ethyl acetate-hexane (1:19-65:35, v/v) to give a white solid. This was subjected to basic silica gel column chromatography, and eluted with ethyl acetate-hexane (1:19 to ethyl acetate alone, v/v) to give a white solid. Recrystallization from ethyl acetate-hexane gave N-(aminosulfonyl)-3-[2-{[3-chloro-5-(trifluoromethyl)pyridin-2-yl]oxy}-4-(2-methoxyethoxy)phenyl]-N-(2-methoxyethyl)propanamide (65 mg, yield: 29%) as white crystals. melting point 101.0-101.1° C.

### Example 214

To a solution of 3-[2-{[3-chloro-5-(trifluoromethyl)pyridin-2-yl]oxy}-4-(2-methoxyethoxy)phenyl]propanoic acid (507 mg) in acetonitrile (6 ml) were added 1-ethyl-3-(3-dimethylaminopropyl)carbodiimide (0.25 g), 4-dimethylaminopyridine (335 mg) and N-pentylsulfamide (202 mg), and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (hexane alone to 9:1, v/v) to give a white solid. The obtained solid was subjected to silica gel column chromatography, and eluted with ethyl acetate-hexane (1:9-1:4, v/v) to give a white solid. Recrystallization from ethyl acetate-hexane gave 3-[2-{[3-chloro-5-(trifluoromethyl)pyridin-2-yl]oxy}-4-(2-methoxyethoxy)phenyl]-N-[(pentylamino)sulfonyl]propanamide (14 mg, yield: 2%) as white crystals. melting point 153.0-153.5° C.

¹H-NMR (300 MHz, CDCl₃) δ:0.85-0.93 (3H, m), 1.20-1.37 (4H, m), 1.47 (2H, tt, J=7.3, 7.2 Hz), 2.57 (2H, t, J=7.2 Hz), 2.71 (2H, q, J=6.8 Hz), 2.92 (2H, t, J=7.3 Hz), 3.42 (3H, s), 3.68-3.74 (2H, m), 4.03-4.08 (2H, m); 4.99 (1H, t, J=6.3 Hz), 6.55 (1H, d, J=2.4 Hz), 6.78 (1H, dd, J=8.5, 2.4 Hz), 7.19 (1H, d, J=8.5 Hz), 8.07 (1H, d, J=2.3 Hz), 8.35 (1H, dd, J=2.0, 0.8 Hz), 8.76 (1H, s).

### Example 215

To a solution of (2E)-3-[2-{[3-chloro-5-(trifluoromethyl)pyridin-2-yl]oxy}-4-(2-methoxyethoxy)phenyl]acrylic acid (837 mg) in acetonitrile (5 ml) were added 1-ethyl-3-(3-dimethylaminopropyl)carbodiimide hydrochloride (515 mg), 4-dimethylaminopyridine (385 mg) and N-pentylsulfamide (336 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated. The obtained residue was subjected to silica gel column chromatography, and eluted with ethyl acetate-hexane (1:9-7:3, v/v) to give a white solid. Recrystallization from ethyl acetate-hexane gave white crystals. The crystals were recrystallized from ethanol-water to give (2E)-3-[2-{[3-chloro-5-(trifluoromethyl)pyridin-2-yl]oxy}-4-(2-methoxyethoxy)phenyl]-N-[(pentylamino)sulfonyl]acrylamide (104 mg, yield: 9%) as white crystals. melting point 120-122° C.

### Example 216

To a solution of (2Z)-3-[2-{[3-chloro-5-(trifluoromethyl)pyridin-2-yl]oxy}-4-(2-methoxyethoxy)phenyl]acrylic acid (300 mg) in dichloromethane (10 ml) were added 1-ethyl-3-(3-dimethylaminopropyl)carbodiimide (0.20 g), 4-dimethylaminopyridine (236 mg) and pentane-1-sulfonamide (115 mg), and the mixture was stirred under ice-cooling for 1 hr. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-45:55, v/v) to give a white solid. Recrystallization from ethyl acetate-hexane gave (2Z)-3-[2-{[3-chloro-5-(trifluoromethyl)pyridin-2-yl]oxy}-4-(2-methoxyethoxy)phenyl]-N-(pentylsulfonyl)acrylamide (139 mg, yield: 35%) as white crystals. melting point 104-106° C.

### Example 217

To a solution of (2E)-3-(4-(2-{[tert-butyl(diphenyl)silyl]oxy}ethoxy)-2-{[3-chloro-5-(trifluoromethyl)pyridin-2-yl]oxy}phenyl)acrylic acid (789 mg) in acetonitrile (6 ml) were added 1-ethyl-3-(3-dimethylaminopropyl)carbodiimide hydrochloride (356 mg), 4-dimethylaminopyridine (209 mg) and pentane-1-sulfonamide (176 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated. The obtained residue was subjected to silica gel column chromatography, and eluted with ethyl acetate-hexane (1:19-2:3, v/v) to give (2E)-3-(4-(2-{[tert-butyl(diphenyl)silyl]oxy}ethoxy)-2-{[3-chloro-5-(trifluoromethyl)pyridin-2-yl]oxy}phenyl)-N-(pentylsulfonyl)acrylamide (515 mg, yield: 54%) as a colorless solid. Recrystallization from ethyl acetate-hexane gave white crystal as dihydrate. melting point 108.0-108.5° C.

### Example 218

To a solution of (2E)-3-(4-(2-{[tertbutyl(diphenyl)silyl]oxy}ethoxy)-2-{[3-chloro-5-(trifluoromethyl)pyridin-2-yl]oxy}phenyl)-N-(pentylsulfonyl)acrylamide (490 mg) in tetrahydrofuran (6 ml) was added a tetra-n-butylammonium fluoride 1.0M-tetrahydrofuran solution (1.20 ml), and the mixture was stirred at 50° C. for 20 min. 1N Hydrochloric acid was added to the reaction mixture, and the mixture was diluted with ethyl acetate. The organic layer was washed with water and saturated brine, dried (MgSO₄), filtrated and concentrated. The obtained residue was subjected to silica gel column chromatography, and eluted with ethyl acetate-hexane (15:85 to ethyl acetate alone, v/v) to give a white solid. Recrystallization from ethyl acetate-hexane gave (2E)-3-[2-{[3-chloro-5-(trifluoromethyl)pyridin-2-yl]oxy}-4-(2-hydroxyethoxy)phenyl]-N-(pentylsulfonyl)acrylamide monohydrate (234 mg, yield: 69%) as white crystals. melting point 112.5-112.8° C.

### Example 219

To a solution of ethyl (2E)-3-[2-(4-aminophenoxy)-4-(2-methoxyethoxy)phenyl]acrylate (0.92 g) in acetonitrile (10 ml) were added cupric chloride (513 mg) and t-butyl nitrite (0.45 ml) under ice-cooling, and the mixture was stirred overnight at room temperature. Water was added to the reaction mixture, and the mixture was diluted with ethyl acetate. The organic layer was washed with 28% w/w ammonia water, water and saturated brine, dried (MgSO₄), filtrated and concentrated. The obtained residue was subjected to silica gel column chromatography, and eluted with ethyl acetate-hexane (hexane alone to 1:1, v/v). The obtained residue was subjected to silica gel column chromatography, and eluted with ethyl acetate-hexane (1:19-2:3, v/v) to give an orange oil.

To a solution of the obtained oil in tetrahydrofuran (1 ml) and ethanol (1 ml) was added a 1N aqueous sodium hydroxide solution (2.5 ml), and the mixture was stirred at 50° C. for 30 min. After allowing to cool to room temperature, 1N hydrochloric acid (2.5 ml) was added to the reaction mixture, and the mixture was diluted with ethyl acetate. The organic layer was washed with saturated brine, dried (MgSO₄), filtrated and concentrated.

To a solution of obtained residue in acetonitrile (2 ml) were added 1-ethyl-3-(3-dimethylaminopropyl)carbodiimide hydrochloride (339 mg), 4-dimethylaminopyridine (208 mg) and pentane-1-sulfonamide (142 mg), and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (hexane alone to 1:1, v/v) to give a colorless oil. The obtained residue was subjected to reversed-phase high performance liquid chromatography, and eluted with acetonitrile-water (2:3 to acetonitrile alone, v/v) to give 3-[2-(4-chlorophenoxy)-4-(2-methoxyethoxy)phenyl]-N-(pentylsulfonyl)propanamide hemihydrate (30 mg, yield: 2%) as a pale-yellow oil.

¹H-NMR (300 MHz, CDCl₃) δ:0.83-0.92 (3H, m), 1.21-1.41 (4H, m), 1.65-1.78 (2H, m), 2.61 (2H, t, J=7.4 Hz), 2.91 (2H, t, J=7.4 Hz), 3.26-3.37 (2H, m), 3.41 (3H, s), 3.69 (2H, dd, J=5.5, 3.8 Hz), 4.01 (2H, dd, J 5.6, 3.9 Hz), 6.43 (1H, d, J=2.4 Hz), 6.61-6.72 (1H, m), 6.86-6.93 (2H, m), 7.16 (1H, d, J=8.5 Hz), 7.26-7.32 (2H, m).

Then, a pale-yellow solid was obtained. Recrystallization from ethyl acetate-diisopropyl ether gave 3-[4-(2-methoxyethoxy)-2-phenoxyphenyl]-N-(pentylsulfonyl)propanamide (44 mg, yield: 4%) as white crystals. melting point 70.4-70.5° C.

### Example 220

To a solution of (2E)-3-(2-{[3-chloro-5-(trifluoromethyl)pyridin-2-yl]oxy}-4-isopropoxyphenyl)-2-methylacrylic acid (409 mg) in acetonitrile (10 ml) were added 1-ethyl-3-(3-dimethylaminopropyl)carbodiimide hydrochloride (311 mg), 4-dimethylaminopyridine (198 mg) and N-(2-phenylethyl)sulfamide (195 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol gave (2E)-3-(2-{[3-chloro-5-(trifluoromethyl)pyridin-2-yl]oxy}-4-isopropoxyphenyl)-2-methyl-N-{[(2-phenylethyl)amino]sulfonyl}acrylamide (246 mg, yield: 42%) as white crystals. melting point 147.0-147.5° C.

### Example 221

To a solution of (2E)-3-(2-{[3-chloro-5-(trifluoromethyl)pyridin-2-yl]oxy}-4-isopropoxyphenyl)-2-methylacrylic acid (216 mg) in acetonitrile (2.5 ml) were added 1-ethyl-3-(3-dimethylaminopropyl)carbodiimide hydrochloride (154 mg), 4-dimethylaminopyridine (102 mg) and N-(3-methoxypropyl)sulfamide (102 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-hexane gave (2E)-3-(2-{[3-chloro-5-(trifluoromethyl)pyridin-2-yl]oxy}-4-isopropoxyphenyl)-N-{[(3-methoxypropyl)amino]sulfonyl}-2-methylacrylamide (79 mg, yield: 27%) as white crystals. melting point 120.0-121.0° C.

### Example 222

To a solution of (2E)-3-(2-{[3-chloro-5-(trifluoromethyl)pyridin-2-yl]oxy}-4-isopropoxyphenyl)-2-methylacrylic acid (215 mg) in acetonitrile (2.5 ml) were added 1-ethyl-3-(3-dimethylaminopropyl)carbodiimide hydrochloride (155 mg), 4-dimethylaminopyridine (108 mg) and N-(2-isopropoxyethyl)sulfamide (106 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-hexane gave (2E)-3-(2-{[3-chloro-5-(trifluoromethyl)pyridin-2-yl]oxy}-4-isopropoxyphenyl)-N-{[(2-isopropoxyethyl)amino]sulfonyl}-2-methylacrylamide (160 mg, yield: 54%) as white crystals. melting point 137.5-137.7° C.

### Example 223

To a solution of (2E)-3-(2-{[3-chloro-5-(trifluoromethyl)pyridin-2-yl]oxy}-4-isopropoxyphenyl)-2-methylacrylic acid (210 mg) in acetonitrile (2.5 ml) were added 1-ethyl-3-(3-dimethylaminopropyl)carbodiimide hydrochloride (170 mg), 4-dimethylaminopyridine (110 mg) and N-(4-methylcyclohexyl)sulfamide (88 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-(2-{[3-chloro-5-(trifluoromethyl)pyridin-2-yl]oxy}-4-isopropoxyphenyl)-2-methyl-N-{[(4-methylcyclohexyl)amino]sulfonyl}acrylamide (130 mg, yield: 49%) as white crystals. melting point 176.8-177.2° C.

### Example 224

To a solution of (2E)-3-(2-{[3-chloro-5-(trifluoromethyl)pyridin-2-yl]oxy}-4-isopropoxyphenyl)-2-methylacrylic acid (218 mg) in acetonitrile (2.5 ml) were added 1-ethyl-3-(3-dimethylaminopropyl)carbodiimide hydrochloride (161 mg), 4-dimethylaminopyridine (107 mg) and N-pentylsulfamide (75 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-(2-{[3-chloro-5-(trifluoromethyl)pyridin-2-yl]oxy}-4-isopropoxyphenyl)-2-methyl-N-[(pentylamino)sulfonyl]acrylamide (176 mg, yield: 69%) as white crystals. melting point 137.8-138.5° C.

### Example 225

To a solution of (2E)-3-(2-{[3-chloro-5-(trifluoromethyl)pyridin-2-yl]oxy}-4-isopropoxyphenyl)-2-methylacrylic acid (210 mg) in acetonitrile (2.5 ml) were added 1-ethyl-3-(3-dimethylaminopropyl)carbodiimide hydrochloride (159 mg), 4-dimethylaminopyridine (112 mg) and N-[2-(2-thienyl)ethyl]sulfamide (89 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-(2-{[3-chloro-5-(trifluoromethyl)pyridin-2-yl]oxy}-4-isopropoxyphenyl)-2-methyl-N-({[2-(2-thienyl)ethyl]amino}sulfonyl)acrylamide (181 mg, yield: 69%) as white crystals. melting point 130-131° C.

### Example 226

To a solution of (2E)-3-[2-{[3-chloro-5-(trifluoromethyl)pyridin-2-yl]oxy}-4-(2-methoxyethoxy)phenyl]-2-methylacrylic acid (220 mg) in acetonitrile (2.5 ml) were added 1-ethyl-3-(3-dimethylaminopropyl)carbodiimide hydrochloride (172 mg), 4-dimethylaminopyridine (117 mg) and N-pentylsulfamide (74 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-[2-{[3-chloro-5-(trifluoromethyl)pyridin-2-yl]oxy}-4-(27-methoxyethoxy)phenyl]-2-methyl-N-[(pentylamino)sulfonyl]acrylamide (167 mg, yield: 65%) as white crystals. melting point 152.5-153.0° C.

### Example 227

To a solution of (2E)-3-[2-{[3-chloro-5-(trifluoromethyl)pyridin-2-yl]oxy}-4-(2-methoxyethoxy)phenyl]-2-methylacrylic acid (218 mg) in acetonitrile (2.5 ml) were added 1-ethyl-3-(3-dimethylaminopropyl)carbodiimide hydrochloride (163 mg), 4-dimethylaminopyridine (109 mg) and N-(3-methoxypropyl)sulfamide (102 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-[2-{[3-chloro-5-(trifluoromethyl)pyridin-2-yl]oxy}-4-(2-methoxyethoxy)phenyl]-N-{[(3-methoxypropyl)amino]sulfonyl}-2-methylacrylamide (83 mg, yield: 28%) as white crystals. melting point 121-122° C.

### Example 228

To a solution of (2E)-3-[2-{[3-chloro-5-(trifluoromethyl)pyridin-2-yl]oxy}-4-(2-methoxyethoxy)phenyl]acrylic acid (420 mg) in acetonitrile (5 ml) were added 1-ethyl-3-(3-dimethylaminopropyl)carbodiimide hydrochloride (276 mg), 4-dimethylaminopyridine (167 mg) and N-(2-phenylethyl)sulfamide (188 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated. The obtained residue was subjected to silica gel column chromatography, and eluted with ethyl acetate-hexane (1:19-45:55, v/v) to give a white solid. Recrystallization from ethyl acetate-hexane gave white crystals. Recrystallization from ethanol-hexane gave (2E)-3-[2-{[3-chloro-5-(trifluoromethyl)pyridin-2-yl]oxy}-4-(2-methoxyethoxy)phenyl]-N-{[(2-phenylethyl)amino]sulfonyl}acrylamide (75 mg, yield: 13%) as white crystals. melting point 85.0-85.5° C.

### Example 229

To a solution of (2E)-3-[2-{[3-chloro-5-(trifluoromethyl)pyridin-2-yl]oxy}-4-(2-methoxyethoxy)phenyl]acrylic acid (419 mg) in acetonitrile (10 ml) were added 1-ethyl-3-(3-dimethylaminopropyl)carbodiimide hydrochloride (304 mg), 4-dimethylaminopyridine (193 mg) and 4-chlorobenzenesulfonamide (174 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-N-[(4-chlorophenyl)sulfonyl]-3-[2-{[3-chloro-5-(trifluoromethyl)pyridin-2-yl]oxy}-4-(2-methoxyethoxy)phenyl]acrylamide (386 mg, yield: 72%) as white crystals. melting point 182.0-182.5° C.

### Example 230

To a solution of (2E)-3-[2-{[3-chloro-5-(trifluoromethyl)pyridin-2-yl]oxy}-4-(2-methoxyethoxy)phenyl]-2-methylacrylic acid (270 mg) in acetonitrile (10 ml) were added 1-ethyl-3-(3-dimethylaminopropyl)carbodiimide hydrochloride (181 mg), 4-dimethylaminopyridine (114 mg) and 4-chlorobenzenesulfonamide (114 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-N-[(4-chlorophenyl)sulfonyl]-3-[2-{[3-chloro-5-(trifluoromethyl)pyridin-2-yl]oxy}-4-(2-methoxyethoxy)phenyl]-2-methylacrylamide (194 mg, yield: 54%) as white crystals. melting point 145.7-145.8° C.

### Example 231

To a solution of (2E)-3-(2-{[3-chloro-5-(trifluoromethyl)pyridin-2-yl]oxy}-4-isopropoxyphenyl)-2-methylacrylic acid (216 mg) in acetonitrile (2.5 ml) were added 1-ethyl-3 (3-dimethylaminopropyl)carbodiimide hydrochloride (163 mg), 4-dimethylaminopyridine (143 mg) and N-(tetrahydrofuran-2-ylmethyl)sulfamide (106 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-hexane gave (2E)-3-(2-{[3-chloro-5-(trifluoromethyl)pyridin-2-yl]oxy}-4-isopropoxyphenyl)-2-methyl-N-{[(tetrahydrofuran-2-ylmethyl)amino]sulfonyl}acrylamide (119 mg, yield: 39%) as white crystals. melting point 125.3-126.5° C.

### Example 232

To a solution of (2E)-3-[2-{[3-chloro-5-(trifluoromethyl)pyridin-2-yl]oxy}-4-(2-methoxyethoxy)phenyl]acrylic acid (424 mg) in acetonitrile (5 ml) were added 1-ethyl-3-(3-dimethylaminopropyl)carbodiimide hydrochloride (319 mg), 4-dimethylaminopyridine (218 mg) and 3-chlorobenzenesulfonamide (191 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-N-[(3-chlorophenyl)sulfonyl]-3-[2-{[3-chloro-5-(trifluoromethyl)pyridin-2-yl]oxy}-4-(2-methoxyethoxy)phenyl]acrylamide (587 mg, yield: 100%) as white crystals. melting point 157.0-158.0° C.

### Example 233

To a solution of (2E)-3-[2-{[3-chloro-5-(trifluoromethyl)pyridin-2-yl]oxy}-4-(2-methoxyethoxy)phenyl]-2-methylacrylic acid (446 mg) in acetonitrile (5 ml) were added 1-ethyl-3-(3-dimethylaminopropyl) carbodiimide hydrochloride (327 mg), 4-dimethylaminopyridine (192 mg) and 3-chlorobenzenesulfonamide (179 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethyl acetate-hexane gave (2E)-N-[(3-chlorophenyl)sulfonyl]-3-[2-{[3-chloro-5-(trifluoromethyl)pyridin-2-yl]oxy}-4-(2-methoxyethoxy)phenyl]-2-methylacrylamide (250 mg, yield: 44%) as white crystals. melting point 89-92° C.

### Example 234

To a solution of (2E)-3-[2-{[3-chloro-5-(trifluoromethyl)pyridin-2-yl]oxy}-4-(2-methoxyethoxy)phenyl]acrylic acid (845 mg) in acetonitrile (10 ml) were added 1-ethyl-3-(3-dimethylaminopropyl) carbodiimide hydrochloride (635 mg), 4-dimethylaminopyridine (331 mg) and 4-(trifluoromethyl)benzenesulfonamide (446 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethyl acetate-hexane gave (2E)-3-[2-{[3-chloro-5-(trifluoromethyl)pyridin-2-yl]oxy}-4-(2-methoxyethoxy)phenyl]-N-{[4-(trifluoromethyl)phenyl]sulfonyl}acrylamide (794 mg, yield: 64%) as white crystals. melting point 186.3-186.5° C.

### Example 235

To a solution of (2E)-3-[2-{[3-chloro-5-(trifluoromethyl)pyridin-2-yl]oxy}-4-(2-methoxyethoxy)phenyl]acrylic acid (834 mg) in acetonitrile (10 ml) were added 1-ethyl-3-(3-dimethylaminopropyl)carbodiimide hydrochloride (516 mg), 4-dimethylaminopyridine (331 mg) and 2-chlorobenzenesulfonamide (383 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-N-[(2-chlorophenyl)sulfonyl]-3-[2-{[3-chloro-5-(trifluoromethyl)pyridin-2-yl]oxy}-4-(2-methoxyethoxy)phenyl]acrylamide (822 mg, yield: 70%) as white crystals. melting point 168-169° C.

### Example 236

To a solution of (2E)-3-[2-{[3-chloro-5-(trifluoromethyl)pyridin-2-yl]oxy}-4-(2-methoxyethoxy)phenyl]acrylic. acid (833 mg) in acetonitrile (10 ml) were added 1-ethyl-3-(3-dimethylaminopropyl)carbodiimide hydrochloride (529 mg), 4-dimethylaminopyridine (343 mg) and p-toluenesulfonamide (372 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethyl acetate-hexane gave (2E)-3-[2-{[3-chloro-5-(trifluoromethyl)pyridin-2-yl]oxy}-4-(2-methoxyethoxy)phenyl]-N-[(4-methylphenyl)sulfonyl]acrylamide (831 mg, yield: 73%) as white crystals. melting point 102.5-106.5° C.

### Example 237

To a solution of (2E)-3-[2-{[3-chloro-5-(trifluoromethyl)pyridin-2-yl]oxy}-4-(2-methoxyethoxy)phenyl]acrylic acid (843 mg) in acetonitrile (10 ml) were added 1-ethyl-3-(3-dimethylaminopropyl)carbodiimide hydrochloride (629 mg), 4-dimethylaminopyridine (346 mg) and 4-fluorobenzenesulfonamide (350 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-[2-{[3-chloro-5-(trifluoromethyl)pyridin-2-yl]oxy}-4-(2-methoxyethoxy)phenyl]-N-[(4-fluorophenyl)sulfonyl]acrylamide (670 mg, yield: 58%) as white crystals. melting point 183.0-184.0° C.

### Example 238

To a solution of (2E)-3-[2-{[3-chloro-5-(trifluoromethyl)pyridin-2-yl]oxy}-4-(2-methoxyethoxy)phenyl]acrylic acid (843 mg) in acetonitrile (10 ml) were added 1-ethyl-3-(3-dimethylaminopropyl)carbodiimide hydrochloride (600 mg), 4-dimethylaminopyridine (352 mg) and benzenesulfonamide (307 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-[2-{[3-chloro-5-(trifluoromethyl)pyridin-2-yl]oxy}-4-(2-methoxyethoxy)phenyl]-N-(phenylsulfonyl)acrylamide (774 mg, yield: 71%) as white crystals. melting point 135.5-136.0° C.

### Example 239

To a solution of (2E)-3-[2-{[3-chloro-5-(trifluoromethyl)pyridin-2-yl]oxy}-4-(2-methoxyethoxy)phenyl]acrylic acid (206 mg) in acetonitrile (5 ml) were added 1-ethyl-3-(3-dimethylaminopropyl)carbodiimide hydrochloride (276 mg), 4-dimethylaminopyridine (97 mg) and 2-pyridinesulfonamide (71 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-[2-{[3-chloro-5-(trifluoromethyl)pyridin-2-yl]oxy}-4-(2-methoxyethoxy)phenyl]-N-(pyridin-2-ylsulfonyl)acrylamide (36 mg, yield: 13%) as white mica crystals. melting point 164.0-165.0° C.

### Example 240

To a solution of (2E)-3-[2-{[3-chloro-5-(trifluoromethyl)pyridin-2-yl]oxy}-4-(2-methoxyethoxy)phenyl]acrylic acid (846 mg) in acetonitrile (10 ml) were added 1-ethyl-3-(3-dimethylaminopropyl)carbodiimide hydrochloride (620 mg), 4-dimethylaminopyridine (339 mg) and 3-pyridinesulfonamide (314 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-[2-{[3-chloro-5-(trifluoromethyl)pyridin-2-yl]oxy}-4-(2-methoxyethoxy)phenyl]-N-(pyridin-3-ylsulfonyl)acrylamide (557 mg, yield: 51%) as white crystals. melting point 165-167° C.

### Example 241

To a solution of (2E)-3-[2-{[3-chloro-5-(trifluoromethyl)pyridin-2-yl]oxy}-4-(2-methoxyethoxy)phenyl]acrylic acid (851 mg) in acetonitrile (10 ml) were added 1-ethyl-3-(3-dimethylaminopropyl)carbodiimide hydrochloride (573 mg), 4-dimethylaminopyridine (336 mg) and m-toluenesulfonamide (375 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-[2-{[3-chloro-5-(trifluoromethyl)pyridin-2-yl]oxy}-4-(2-methoxyethoxy)phenyl]-N-[(3-methylphenyl)sulfonyl]acrylamide (737 mg, yield: 63%) as white crystals. melting point 147.0-147.5° C.

### Example 242

To a solution of (2E)-3-[2-{[3-chloro-5-(trifluoromethyl)pyridin-2-yl]oxy}-4-(2-methoxyethoxy)phenyl]acrylic acid (837 mg) in acetonitrile (10 ml) were added 1-ethyl-3-(3-dimethylaminopropyl)carbodiimide hydrochloride (625 mg), 4-dimethylaminopyridine (339 mg) and 3-cyanobenzenesulfonamide (369 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethyl acetate-hexane gave (2E)-3-[2-{[3-chloro-5-(trifluoromethyl)pyridin-2-yl]oxy}-4-(2-methoxyethoxy)phenyl]-N-[(3-cyanophenyl)sulfonyl]acrylamide (931 mg, yield: 80%) as white crystals. melting point 195-197° C.

### Example 243

To a solution of (2E)-3-[2-{[3-chloro-5-(trifluoromethyl)pyridin-2-yl]oxy}-4-(2-methoxyethoxy)phenyl]acrylic acid (837 mg) in acetonitrile (10 ml) were added 1-ethyl-3-(3-dimethylaminopropyl)carbodiimide hydrochloride (632 mg), 4-dimethylaminopyridine (332 mg) and trifluoromethanesulfonamide (306 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethyl acetate-hexane gave (2E)-3-[2-{[3-chloro-5-(trifluoromethyl)pyridin-2-yl]oxy}-4-(2-methoxyethoxy)phenyl]-N-[(trifluoromethyl)sulfonyl]acrylamide (428 mg, yield: 39%) as white crystals. melting point 125.5-128.0° C.

### Example 244

To a solution of (2E)-3-[2-{[3-chloro-5-(trifluoromethyl)pyridin-2-yl]oxy}-4-(2-methoxyethoxy)phenyl]-2-methylacrylic acid (860 mg) in acetonitrile (10 ml) were added 1-ethyl-3-(3-dimethylaminopropyl)carbodiimide hydrochloride (795 mg), 4-dimethylaminopyridine (316 mg) and 2,2,2-trifluoroethanesulfonamide (320 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-[2-{[3-chloro-5-(trifluoromethyl)pyridin-2-yl]oxy}-4-(2-methoxyethoxy)phenyl]-2-methyl-N-[(2,2,2-trifluoroethyl)sulfonyl]acrylamide (905 mg, yield: 80%) as white crystals. melting point 159.0-159.5° C.

### Example 245

To a solution of (2E)-3-[2-{[3-chloro-5-(trifluoromethyl)pyridin-2-yl]oxy}-4-(2-methoxyethoxy)phenyl]-2-methylacrylic acid (871 mg) in acetonitrile (10 ml) were added 1-ethyl-3-(3-dimethylaminopropyl)carbodiimide hydrochloride (814 mg), 4-dimethylaminopyridine (355 mg) and trifluoromethanesulfonamide (305 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-[2-{[3-chloro-5-(trifluoromethyl)pyridin-2-yl]oxy}-4-(2-methoxyethoxy)phenyl]-2-methyl-N-[(trifluoromethyl)sulfonyl]acrylamide (980 mg, yield: 86%) as white crystals. melting point 146.0-147.0° C.

### Example 246

To a solution of (2E)-3-[2-{[3-chloro-5-(trifluoromethyl)pyridin-2-yl]oxy}-4-(2-methoxyethoxy)phenyl]acrylic acid (840 mg) in acetonitrile (10 ml) were added 1-ethyl-3-(3-dimethylaminopropyl)carbodiimide hydrochloride (634 mg), 4-dimethylaminopyridine (370 mg) and 2,2,2-trifluoroethanesulfonamide (324 mg), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated to give a white solid. Recrystallization from ethanol-water gave (2E)-3-[2-{[3-chloro-5-(trifluoromethyl)pyridin-2-yl]oxy}-4-(2-methoxyethoxy)phenyl]-N-[(2,2,2-trifluoroethyl)sulfonyl]acrylamide (847 mg, yield: 76%) as white crystals. melting point 146.0-147.5° C.

### Example 247

To a solution of (2E)-3-{2-{[3-chloro-5-(trifluoromethyl)pyridin-2-yl]oxy}-4-[3-(methylsulfonyl)propoxy]phenyl}acrylic acid (474 mg) in acetonitrile (10 ml) were added 1-ethyl-3-(3-dimethylaminopropyl)carbodiimide hydrochloride (373 mg), 4-dimethylaminopyridine (186 mg) and pentane-1-sulfonamide (144 mg), and the mixture was stirred overnight at room temperature. 1N Hydrochloric acid was added to the reaction mixture, and the resulting solid was collected by filtration and washed with water to give white solid. Recrystallization from ethanol-water gave (2E)-3-{2-{[3-chloro-5-(trifluoromethyl)pyridin-2-yl]oxy}-4-[3-(methylsulfonyl)propoxy]phenyl}-N-(pentylsulfonyl)acrylamide (326 mg, yield: 56%) as white crystals. melting point 194-196° C.

### Example 248

To a solution of (2E)-3-[2-{[3-chloro-5-(trifluoromethyl)pyridin-2-yl]oxy}-4-(3-hydroxy-3-methylbutoxy)phenyl]acrylic acid (446 mg) in acetonitrile (5 ml) were added 1-ethyl-3-(3-dimethylaminopropyl)carbodiimide hydrochloride (360 mg), 4-dimethylaminopyridine (230 mg) and pentane-1-sulfonamide (150 mg), and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:9-3:2, v/v) to give a white solid. Recrystallization from ethyl acetate-hexane gave (2E)-3-[2-{[3-chloro-5-(trifluoromethyl)pyridin-2-yl]oxy}-4-(3-hydroxy-3-methylbutoxy)phenyl]-N-(pentylsulfonyl)acrylamide (242 mg, yield: 42%) as white crystals. melting point 135.5-136.0° C.

### Example 249

To a solution of ethyl (2E)-3-{2-{[3-chloro-5-(trifluoromethyl)pyridin-2-yl]oxy}-4-[3-(methoxymethoxy)-1,1-dimethylpropoxy]phenyl}acrylate (975 mg) in tetrahydrofuran (2.5 ml) and ethanol (2.5 ml) was added a 1N aqueous sodium hydroxide solution (5.0 ml), and the mixture was stirred at 50° C. for 30 min. After allowing to cool to room temperature, 1N hydrochloric acid (5.0 ml) was added to the reaction mixture, and the mixture was extracted with ethyl acetate. The organic layer was washed with water and saturated brine, dried (MgSO₄), filtrated and concentrated to give a pale-yellow oil.

To a solution of the obtained oil in acetonitrile (10 ml) were added 1-ethyl-3-(3-dimethylaminopropyl)carbodiimide hydrochloride (767 mg), 4-dimethylaminopyridine (293 mg) and pentane-1-sulfonamide (262 mg), and the mixture was stirred overnight at room temperature. 1N Hydrochloric acid was added to the reaction mixture, and the mixture was diluted with ethyl acetate. The organic layer was washed with 1N hydrochloric acid, a saturated aqueous sodium hydrogencarbonate solution and saturated brine, dried (MgSO₄), filtrated and concentrated. The obtained residue was subjected to silica gel column chromatography, and eluted with ethyl acetate-hexane (1:19-1:1, v/v). The obtained residue was subjected to reversed-phase high performance liquid chromatography, and eluted with acetonitrile-water (2:3 to acetonitrile alone, v/v) to give (2E)-3-{2-{[3-chloro-5-(trifluoromethyl)pyridin-2-yl]oxy}-4-[3-(methoxymethoxy)-1,1-dimethylpropoxy]phenyl}-N-(pentylsulfonyl)acrylamide (222 mg, yield: 21%) as a colorless oil.

¹H-NMR (300 MHz, CDCl₃) δ:0.73-0.97 (3H, m), 1.18-1.36 (4H, m), 1.38 (6H, s), 1.65-1.89 (2H, m), 2.03 (2H, t, J=7.1 Hz), 3.35 (3H, s), 3.37-3.54 (2H, m), 3.74 (2H, t, J=7.2 Hz), 4.61 (2H, s), 6.45 (1H, d, J=115.6 Hz), 6.77 (1H, d, J=2.3 Hz), 6.93 (1H, d, J=8.3 Hz), 7.58 (1H, d, J=8.7 Hz), 7.80 (1H, d, J=15.6 Hz), 7.92-8.08 (1H, m), 8.18-8.29 (1H, m).

### Example 250

To a solution of (2E)-3-[2-{[3-chloro-5-(trifluoromethyl)pyridin-2-yl]oxy}-4-(2-hydroxy-2-methylpropoxy)phenyl]acrylic acid (3.62 g) in acetonitrile (40 ml) were added 1-ethyl-3-(3-dimethylaminopropyl)carbodiimide hydrochloride (3.21 g), 4-dimethylaminopyridine (1.54 g) and pentane-1-sulfonamide (1.27 g), and the mixture was stirred overnight at room temperature. 1N Hydrochloric acid was added to the reaction mixture, and the mixture was diluted with ethyl acetate. The organic layer was washed with 1N hydrochloric acid and saturated brine, dried (MgSO₄), filtrated and concentrated. The obtained residue was subjected to silica gel column chromatography, and eluted with ethyl acetate-hexane (1: 9-1:1, v/v). The obtained residue was subjected to silica gel column chromatography, and eluted with ethyl acetate-hexane (1:4-1:1, v/v). The obtained residue was subjected to silica gel column chromatography, and eluted with ethyl acetate-hexane (1:9-9:1, v/v). The residue was triturated with diisopropyl ether to give (2E)-3-[2-{[3-chloro-5-(trifluoromethyl)pyridin-2-yl]oxy}-4-(2-hydroxy-2-methylpropoxy)phenyl]-N-(pentylsulfonyl)acrylamide 0.10 diisopropyl ether-0.19 ethyl acetate complex (424 mg, yield: 9%) as a white solid. Recrystallization from acetone-hexane gave white crystals as 0.23 acetone solvate. melting point 98-100° C.

¹H-NMR (300 MHz, CDCl₃) δ:0.86-0.94 (3H, m), 1.21-1.45 (4H, m), 1.34 (6H, s), 1.70-1.92 (2H, m), 2.08 (1H, s), 3.23-3.56 (2H, m), 3.82 (2H, s), 6.38 (1H, d, J=15.4 Hz), 6.71 (1H, d, J=2.5 Hz), 6.91 (1H, dd, J=8.8, 2.5 Hz), 7.61 (1H, d, J=8.8 Hz), 7.78 (1H, d, J=15.4 Hz), 8.03 (1H, d, J=2.2 Hz), 8.25 (1H, dd, J=2.2, 0.8 Hz).

### Example 251

To a solution of (2E)-3-[2-{[3-chloro-5-(trifluoromethyl)pyridin-2-yl]oxy}-4-(3-methoxypropoxy)phenyl]acrylic acid (439 mg) in acetonitrile (10 ml) were added 1-ethyl-3-(3-dimethylaminopropyl)carbodiimide hydrochloride (356 mg), 4-dimethylaminopyridine (206 mg) and pentane-1-sulfonamide (151 mg), and the mixture was stirred overnight at room temperature. 1N Hydrochloric acid was added to the reaction mixture, and the mixture was diluted with ethyl acetate. The organic layer was washed with 1N hydrochloric acid, and saturated brine, dried (MgSO₄), filtrated and concentrated. The obtained residue was subjected to silica gel column chromatography, and eluted with ethyl acetate-hexane (1:19-1:1, v/v) to give a white solid. Recrystallization from ethyl acetate-hexane gave (2E)-3-[2-{[3-chloro-5-(trifluoromethyl)pyridin-2-yl]oxy}-4-(3-methoxypropoxy)phenyl]-N-(pentylsulfonyl)acrylamide (149 mg, yield: 26%) as white crystals. melting point 116.5-118.0° C.

### Example 252

To a solution of 3-[2-{[3-chloro-5-(trifluoromethyl)pyridin-2-yl]oxy}-4-(2-methoxyethoxy)phenyl]-2,2-dimethylpropanoic acid (1.21 g) in tetrahydrofuran (25 ml) was added N,N′-carbonyldiimidazole (877 mg), and the mixture was heated under reflux for 1 hr. After allowing to cool to room temperature, pentane-1-sulfonamide (817 mg) and 1,8-diazabicyclo[5.4.0]undec-7-ene (0.80 ml) were added to the reaction mixture, and the mixture was stirred for 3 hr. 1N Hydrochloric acid was added to the reaction mixture, and the mixture was diluted with ethyl acetate. The organic layer was washed with 1N hydrochloric acid and saturated brine, dried (MgSO₄), filtrated and concentrated. The obtained residue was subjected to silica gel column chromatography, and eluted with ethyl acetate-hexane (hexane alone to 2:3, v/v) to give 3-[2-{[3-chloro-5-(trifluoromethyl)pyridin-2-yl]oxy}-4-(2-methoxyethoxy)phenyl]-2,2-dimethyl-N-(pentylsulfonyl)propanamide (1.51 g, yield: 96%) as a colorless oil.

¹H-NMR (300 MHz, CDCl₃) δ:0.82-0.95 (3H, m), 1.25 (6H, s), 1.28-1.48 (4H, m), 1.75 (2H, tt, J=7.6, 7.6 Hz), 2.76 (2H, s), 3.34-3.42 (2H, m), 3.43 (3H, s), 3.73 (2H, dd, J=5.4, 3.9 Hz), 4.09 (2H, dd, J=5.5, 4.0 Hz), 6.68 (1H, d, J=2.4 Hz), 6.82 (1H, dd, J=8.7, 2.6 Hz), 7.20 (1H, d, J=8.7 Hz), 7.82 (1H, br. s.), 8.01 (1H, d, J=2.3 Hz), 8.28 (1H, dd, J=2.1, 0.9 Hz)

### Example 253

To a solution of 3-[2-{[3-chloro-5-(trifluoromethyl)pyridin-2-yl]oxy}-4-(2-methoxyethoxy)phenyl]-2,2-dimethyl-N-(pentylsulfonyl)propanamide (1.23 g) in methanol (2 ml) was added a 1N aqueous sodium hydroxide solution (2.12 ml) at room temperature, and the reaction mixture was concentrated. The obtained residue was washed with cold methanol to give sodium {3-[2-{[3-chloro-5-(trifluoromethyl)pyridin-2-yl]oxy}-4-(2-methoxyethoxy)phenyl]-2,2-dimethylpropanoyl}(pentylsulfonyl)azanide (982 mg, yield: 77%) as white crystals. melting point 190.5-191.0° C.

### Example 254

To a solution of (2E)-3-{2-{[3-chloro-5-(trifluoromethyl)pyridin-2-yl]oxy}-4-[3-(2-methoxyethoxy)propoxy]phenyl}acrylic acid (2.45 g) in acetonitrile (50 ml) were added 1-ethyl-3-(3-dimethylaminopropyl)carbodiimide hydrochloride (1.97 g), 4-dimethylaminopyridine (1.26 g) and pentane-1-sulfonamide (788 mg), and the mixture was stirred overnight at room temperature. 1N Hydrochloric acid was added to the reaction mixture, and the mixture was diluted with ethyl acetate. The organic layer was washed with 1N hydrochloric acid, and saturated brine, dried (MgSO₄), filtrated and concentrated to give a white solid. Recrystallization from ethyl acetate-hexane gave (2E)-3-{2-{[3-chloro-5-(trifluoromethyl)pyridin-2-yl]oxy}-4-[3-(2-methoxyethoxy)propoxy]phenyl}-N-(pentylsulfonyl)acrylamide (1.70 g, yield: 54%) as white crystals. melting point 103.0-105.0° C.

### Example 255

To a solution of (2E)-3-{2-{[3-chloro-5-(trifluoromethyl)pyridin-2-yl]oxy}-4-[3-(2-methoxyethoxy)propoxy]phenyl}-N-(pentylsulfonyl)acrylamide (569 mg) in tetrahydrofuran (10 ml) and methanol (10 ml) was added a palladium-activated carbon ethylenediamine complex (61 mg), and the mixture was stirred under a hydrogen atmosphere at room temperature for 12.5 hr. The reaction mixture was filtrated, and the filtrate was concentrated. The obtained residue was dissolved in tetrahydrofuran (10 ml) and methanol (10 ml), a palladium-activated carbon ethylenediamine complex (131 mg) was added, and the mixture was stirred under a hydrogen atmosphere at room temperature for 3 hr. The reaction mixture was filtrated and the filtrate was concentrated to give a white solid. Recrystallization from ethyl acetate-hexane gave white crystals. The obtained crystals were subjected to silica gel column chromatography, and eluted with ethyl acetate-hexane (1:9-2:3, v/v) to give a white solid. Recrystallization from ethyl acetate-hexane gave 3-{2-{[3-chloro-5-(trifluoromethyl)pyridin-2-yl]oxy}-4-[3-(2-methoxyethoxy)propoxy]phenyl}-N-(pentylsulfonyl)propanamide (8 mg, yield: 1%) as white crystals. melting point 95.0-95.8° C.

### Example 256

To a solution of (2E)-3-{2-{[3-chloro-5-(trifluoromethyl)pyridin-2-yl]oxy}-4-[2-(2-methoxyethoxy)ethoxy]phenyl}acrylic acid (1.86 g) in acetonitrile (50 ml) were added 1-ethyl-3-(3-dimethylaminopropyl)carbodiimide hydrochloride (1.55 g), 4-dimethylaminopyridine (0.99 g) and pentane-1-sulfonamide (611 mg), and the mixture was stirred overnight at room temperature. 1N Hydrochloric acid was added to the reaction mixture, and the mixture was diluted with ethyl acetate. The organic layer was washed with 1N hydrochloric acid, and saturated brine, dried (MgSO₄), filtrated and concentrated to give a white solid. Recrystallization from ethyl acetate-hexane gave (2E)-3-{2-{[3-chloro-5-(trifluoromethyl)pyridin-2-yl]oxy}-4-[2-(2-methoxyethoxy)ethoxy]phenyl}-N-(pentylsulfonyl)acrylamide (1.01 g, yield: 42%) as white crystals. melting point 81.0-83.0° C.

### Example 257

To a solution of (2E)-3-{2-{[3-chloro-5-(trifluoromethyl)pyridin-2-yl]oxy}-4-[2-(2-methoxyethoxy)ethoxy]phenyl}-N-(pentylsulfonyl)acrylamide (280 mg) in tetrahydrofuran (10 ml) and methanol (10 ml) was added a palladium-activated carbon ethylenediamine complex (60 mg), and the mixture was stirred under a hydrogen atmosphere at room temperature for 8 hr. The reaction mixture was filtrated and the filtrate was concentrated to give a white solid. Recrystallization from ethyl acetate-hexane gave white crystals. The obtained crystals were subjected to silica gel column chromatography, and eluted with ethyl acetate-hexane (1:19′-3:2, v/v) to give a white solid. Recrystallization from ethyl acetate-hexane gave 3-{2-{[3-chloro-5-(trifluoromethyl)pyridin-2-yl]oxy}-4-[2-(2-methoxyethoxy)ethoxy]phenyl}-N-(pentylsulfonyl)propanamide (26 mg, yield: 9%) as white crystals. melting point 76.0-77.5° C.

### Example 258

To a solution of 3-[2-{[3-chloro-5-(trifluoromethyl)pyridin-2-yl]oxy}-4-(3-methoxypropoxy)phenyl]propanoic acid (437 mg) in acetonitrile (10 ml) were added 1-ethyl-3-(3-dimethylaminopropyl)carbodiimide hydrochloride (403 mg), 4-dimethylaminopyridine (179 mg) and pentane-1-sulfonamide (153 mg), and the mixture was stirred overnight at room temperature. 1N Hydrochloric acid was added to the reaction mixture, and the mixture was diluted with ethyl acetate. The organic layer was washed with 1N hydrochloric acid, and saturated brine, dried (MgSO₄), filtrated and concentrated to give a white solid. Recrystallization from ethyl acetate-hexane, then from ethanol-water gave 3-[2-{[3-chloro-5-(trifluoromethyl)pyridin-2-yl]oxy}-4-(3-methoxypropoxy)phenyl]-N-(pentylsulfonyl)propanamide (351 mg, yield: 62%) as white crystals. melting point 115.0-115.2° C.

### Example 259

To a solution of ethyl (2E)-3-{2-{[3-chloro-5-(trifluoromethyl)pyridin-2-yl]oxy}-4-[3-(cyclopropylmethoxy)propoxy]phenyl}acrylate (0.66 g) in tetrahydrofuran (3 ml) and ethanol (3 ml) was added a 1N aqueous sodium hydroxide solution (4 ml), and the mixture was stirred at 50° C. for 1 hr. After allowing to cool to room temperature, 1N hydrochloric acid (4 ml) was added to the reaction mixture, and the mixture was diluted with ethyl acetate. The organic layer was washed with water and saturated brine, dried (MgSO₄), filtrated and concentrated to give a pale yellow oil.

To a solution of the obtained oil in acetonitrile (10 ml) were added 1-ethyl-3-(3-dimethylaminopropyl)carbodiimide hydrochloride (415 mg), 4-dimethylaminopyridine (269 mg) and pentane-1-sulfonamide (164 mg), and the mixture was stirred overnight at room temperature. 1N Hydrochloric acid was added to the reaction mixture, and the mixture was diluted with ethyl acetate. The organic layer was washed with 1N hydrochloric acid, and saturated brine, dried (MgSO₄), filtrated and concentrated. The obtained residue was subjected to silica gel column chromatography, and eluted with ethyl acetate-hexane (1:19-1:1, v/v) to give a white solid. Recrystallization from ethyl acetate-hexane gave (2E)-3-{2-{[3-chloro-5-(trifluoromethyl)pyridin-2-yl]oxy}-4-[3-(cyclopropylmethoxy)propoxy]phenyl}-N-(pentylsulfonyl)acrylamide (301 mg, yield: 46%) as white crystals. melting point 124-125° C.

### Example 260

To a solution of (2E)-3-[2-[(2,4-dichlorobenzyl)oxy]-4-(2-methoxyethoxy)-3-methylphenyl]acrylic acid (86 mg) in acetonitrile (5 ml) were added triethylamine (59 μl) and 2-methyl-6-nitrobenzoic anhydride (109 mg) under ice-cooling, and the mixture was stirred for 30 min. Then, 4-dimethylaminopyridine (51 mg) and pentane-1-sulfonamide (32 mg) were added, and the mixture was stirred overnight while allowing to warm to room temperature. 1N Hydrochloric acid was added to the reaction mixture, and the mixture was diluted with ethyl acetate. The organic layer was washed with 1N hydrochloric acid, and saturated brine, dried (MgSO₄), filtrated and concentrated to give a white solid. Recrystallization from ethyl acetate-hexane gave (2E)-3-[2-[(2,4-dichlorobenzyl)oxy]-4-(2-methoxyethoxy)-3-methylphenyl]-N-(pentylsulfonyl)acrylamide monohydrate (37 mg, yield: 32%) as white crystals. melting point 149-152° C.

In Example 261-Example 423, for the reversed-phase preparative HPLC, a UniPoint system equipped with YMC CombiPrep Pro C18, 50×20 mm, S-5 μm column (Gilson) was used and the mixtures were eluted with 0.1% trifluoroacetic acid-containing acetonitrile-water (5:95-100:0, v/v).

### Example 261

A solution of [2-{[3-chloro-5-(trifluoromethyl)pyridin-2-yl]oxy}-4-(2-methoxyethoxy)phenyl]methanol (30 mg) in dichloromethane (0.50 ml) was cooled to 0° C., a solution of chlorosulfonylisocyanate (12.5 mg) in dichloromethane (0.50 ml) was added and the mixture was stirred for 30 min. A solution of pyridine (19.0 mg) in dichloromethane (0.50 ml) was added to the reaction mixture and the mixture was further stirred at 0° C. for 30 min. A solution of isopropylamine (14.2 mg) in dichloromethane (0.50 ml) was added and the mixture was further stirred overnight while raising the temperature to room temperature. The reaction mixture was extracted with dichloromethane (3.0 ml) and water (2.0 ml), fractionated using a PTFE tube (polytetrafluoroethylene film processing tube, manufactured by Whatman) and the dichloromethane layer was concentrated. The residue was dissolved in DMSO (0.50 ml) and purified by reversed-phase preparative HPLC. 5% Aqueous sodium hydrogencarbonate solution (1.0 ml) was added to the object fraction and the mixture was concentrated. Water (1.0 ml) was added to the residue, and the mixture was extracted with ethyl acetate (3.5 ml) and fractionated by Presep™dehydrating (manufactured by Wako Pure Chemical Industries, Ltd.). The aqueous layer was extracted with ethyl acetate (3.5 ml). The ethyl acetate layers were combined, and the mixture was concentrated with a nitrogen blowing apparatus to give 2-{[3-chloro-5-(trifluoromethyl)pyridin-2-yl]oxy}-4-(2-methoxyethoxy)benzyl [(isopropylamino)sulfonyl]carbamate (25 mg, yield: 57%). LC-MS purity: 100%, m/z=540[M-H⁺], retention time=1.89 min.

¹H-NMR (400 MHz, CDCl₃): 1.06 (6H, d, J=6.4 Hz), 3.38-3.47 (1H, m), 3.42 (3H, s), 3.69-3.75 (2H, m), 4.05-4.13 (2H, m), 4.99-5.10 (3H, m), 6.67 (1H, d, J=2.0 Hz), 6.82 (1H, d, J=6.6 Hz), 7.41 (1H, d, J=8.6 Hz), 7.97 (1H, d, J=1.7 Hz), 8.23 (1H, s).

In the same manner as in Example 261, alcohols of [2-{[3-chloro-5-(trifluoromethyl)pyridin-2-yl]oxy}-4-(2-methoxyethoxy)phenyl]methanol, 3-[2-{[3-chloro-5-(trifluoromethyl)pyridin-2-yl]oxy}-4-(2-methoxyethoxy)phenyl]propan-1-ol, 3-[2-(cyclohexyloxy)-4-(2-methoxyethoxy)phenyl]propan-1-ol, 3-[2-(cyclopropylmethoxy)-4-(2-methoxyethoxy)phenyl]propan-1-ol and 3-[4-(2-methoxyethoxy)-2-(tetrahydrofuran-2-ylmethoxy)phenyl]propan-1-ol were reacted with various amines to give the compounds of Example 262-Example 423.

### Example 262

2-{[3-chloro-5-(trifluoromethyl)pyridin-2-yl]oxy}-4-(2-methoxyethoxy)benzyl {[(2-hydroxyethyl)amino]sulfonyl}carbamate yield: 33%, LC-MS purity: 100%, m/z=542[M-H⁺], retention time=1.68 min.

### Example 263

2-{[3-chloro-5-(trifluoromethyl)pyridin-2-yl]oxy}-4-(2-methoxyethoxy)benzyl {[(2-cyanoethyl)amino]sulfonyl}carbamate yield: 55%, LC-MS purity: 95%, m/z=551[M-H⁺], retention time=1.73 min.

### Example 264

2-{[3-chloro-5-(trifluoromethyl)pyridin-2-yl]oxy}-4-(2-methoxyethoxy)benzyl [(tert-butylamino)sulfonyl]carbamate yield: 50%, LC-MS purity: 95%, m/z=554[M-H⁺], retention time=1.95 min.

### Example 265

2-{[3-chloro-5-(trifluoromethyl)pyridin-2-yl]oxy}-4-(2-methoxyethoxy)benzyl {[(3-hydroxypropyl)amino]sulfonyl}carbamate yield: 16%, LC-MS purity: 100%, m/z=556[M-H⁺], retention time=1.71 min.

### Example 266

2-{[3-chloro-5-(trifluoromethyl)pyridin-2-yl]oxy}-4-(2-methoxyethoxy)benzyl {[(2-methoxyethyl)amino]sulfonyl}carbamate yield: 58%, LC-MS purity: 100%, m/z=556[M-H⁺], retention time=1.77 min.

### Example 267

2-{[3-chloro-5-(trifluoromethyl)pyridin-2-yl]oxy}-4-(2-methoxyethoxy)benzyl [(cyclopentylamino)sulfonyl]carbamate yield: 30%, LC-MS purity: 95%, m/z=566[M-H⁺], retention time=1.95 min.

### Example 268

2-{[3-chloro-5-(trifluoromethyl)pyridin-2-yl]oxy}-4-(2-methoxyethoxy)benzyl {[(1-ethylpropyl)amino]sulfonyl}carbamate yield: 55%, LC-MS purity: 100%, m/z=568[M-H⁺], retention time=2.01 min.

### Example 269

2-{[3-chloro-5-(trifluoromethyl)pyridin-2-yl]oxy}-4-(2-methoxyethoxy)benzyl {[(3-methylbutyl)amino]sulfonyl}carbamate yield: 52%, LC-MS purity: 100%, m/z=568[M-H⁺], retention time=1.98 min.

### Example 270

2-{[3-chloro-5-(trifluoromethyl)pyridin-2-yl]oxy}-4-(2-methoxyethoxy)benzyl {[(1,2-dimethylpropyl)amino]sulfonyl}carbamate yield: 56%, LC-MS purity: 100%, m/z=568[M-H⁺], retention time=2.00 min.

### Example 271

2-{[3-chloro-5-(trifluoromethyl)pyridin-2-yl]oxy}-4-(2-methoxyethoxy)benzyl {[(1-methylbutyl)amino]sulfonyl}carbamate yield: 58%, LC-MS purity: 100%, m/z=568[M-H⁺], retention time=2.00 min.

### Example 272

2-{[3-chloro-5-(trifluoromethyl)pyridin-2-yl]oxy}-4-(2-methoxyethoxy)benzyl {[(4-hydroxybutyl)amino]sulfonyl}carbamate yield: 22%, LC-MS purity: 100%, m/z=570[M-H⁺], retention time=1.74 min.

### Example 273

2-{[3-chloro-5-(trifluoromethyl)pyridin-2-yl]oxy}-4-(2-methoxyethoxy)benzyl [(cyclohexylamino)sulfonyl]carbamate yield: 19%, LC-MS purity: 100%, m/z=580[M-H⁺], retention time=2.00 min.

### Example 274

2-{[3-chloro-5-(trifluoromethyl)pyridin-2-yl]oxy}-4-(2-methoxyethoxy)benzyl {[(tetrahydrofuran-2-ylmethyl)amino]sulfonyl}carbamate yield: 54%, LC-MS purity: 100%, m/z=582[M-H⁺], retention time=1.81 min.

### Example 275

2-{[3-chloro-5-(trifluoromethyl)pyridin-2-yl]oxy}-4-(2-methoxyethoxy)benzyl {[(1,3-dimethylbutyl)amino]sulfonyl}carbamate yield: 57%, LC-MS purity: 100%, m/z=582[M-H⁺], retention time=2.05 min.

### Example 276

2-{[3-chloro-5-(trifluoromethyl)pyridin-2-yl]oxy}-4-(2-methoxyethoxy)benzyl ({[2-(acetylamino)ethyl]amino}sulfonyl)carbamate yield: 23%, LC-MS purity: 100%, m/z=583[M-H⁺], retention time=1.67 min.

### Example 277

2-{[3-chloro-5-(trifluoromethyl)pyridin-2-yl]oxy}-4-(2-methoxyethoxy)benzyl {[(2-isopropoxyethyl)amino]sulfonyl}carbamate yield: 62%, LC-MS purity: 100%, m/z=584[M-H⁺], retention time=1.88 min.

### Example 278

2-{[3-chloro-5-(trifluoromethyl)pyridin-2-yl]oxy}-4-(2-methoxyethoxy)benzyl ({[1-(methoxymethyl)propyl]amino}sulfonyl)carbamate yield: 59%, LC-MS purity: 93%, m/z=584[M-H⁺], retention time=1.91 min.

### Example 279

2-{[3-chloro-5-(trifluoromethyl)pyridin-2-yl]oxy}-4-(2-methoxyethoxy)benzyl {[(3-ethoxypropyl)amino]sulfonyl}carbamate yield: 57%, LC-MS purity: 100%, m/z=584[M-H⁺], retention time=1.87 min.

### Example 280

2-{[3-chloro-5-(trifluoromethyl)pyridin-2-yl]oxy}-4-(2-methoxyethoxy)benzyl ({[3-(methylthio)propyl]amino}sulfonyl)carbamate yield: 67%, LC-MS purity: 100%, m/z=586[M-H⁺], retention time=1.88 min.

### Example 281

2-{[3-chloro-5-(trifluoromethyl)pyridin-2-yl]oxy}-4-(2-methoxyethoxy)benzyl [(benzylamino)sulfonyl]carbamate yield: 46%, LC-MS purity: 100%, m/z=588[M-H⁺], retention time=1.91 min.

¹H-NMR (400 MHz, CDCl₃) δ:3.39 (3H, s), 3.64-3.74 (2H, m), 3.93-4.03 (4H, m), 4.89 (2H, s), 5.49 (1H, s), 6.59 (1H, d, J=1.7 Hz), 6.74 (1H, s), 7.08-7.18 (5H, m), 7.32 (1 H, d, J=8.3 Hz), 7.88 (1H, s), 8.16 (1H, s).

### Example 282

2-{[3-chloro-5-(trifluoromethyl)pyridin-2-yl]oxy}-4-(2-methoxyethoxy)benzyl {[(2-amino-2-oxoethyl)amino]sulfonyl}carbamate yield: 10%, LC-MS purity: 100%, m/z=555[M-H⁺], retention time=1.63 min.

### Example 283

2-{[3-chloro-5-(trifluoromethyl)pyridin-2-yl]oxy}-4-(2-methoxyethoxy)benzyl {[(cyclohexylmethyl)amino]sulfonyl}carbamate yield: 50%, LC-MS purity: 100%, m/z=594[M-H⁺], retention time=2.05 min.

### Example 284

2-{[3-chloro-5-(trifluoromethyl)pyridin-2-yl]oxy}-4-(2-methoxyethoxy)benzyl [(cycloheptylamino)sulfonyl]carbamate yield: 37%, LC-MS purity: 100%, m/z=594[M-H⁺], retention time=2.05 min.

### Example 285

2-{[3-chloro-5-(trifluoromethyl)pyridin-2-yl]oxy}-4-(2-methoxyethoxy)benzyl {[(4-methylcyclohexyl)amino]sulfonyl}carbamate yield: 44%, LC-MS purity: 100%, m/z=594[M-H⁺], retention time=2.06 min.

### Example 286

2-{[3-chloro-5-(trifluoromethyl)pyridin-2-yl]oxy}-4-(2-methoxyethoxy)benzyl {[(3-isopropoxypropyl)amino]sulfonyl}carbamate yield: 74%, LC-MS purity: 95%, m/z=598[M-H⁺], retention time=2.00 min.

### Example 287

2-{[3-chloro-5-(trifluoromethyl)pyridin-2-yl]oxy}-4-(2-methoxyethoxy)benzyl {[(2-phenylethyl)amino]sulfonyl}carbamate yield: 61%, LC-MS purity: 100%, m/z=602[M-H⁺], retention time=1.95 min.

### Example 288

2-{[3-chloro-5-(trifluoromethyl)pyridin-2-yl]oxy}-4-(2-methoxyethoxy)benzyl ({[2-(2-thienyl)ethyl]amino}sulfonyl)carbamate yield: 58%, LC-MS purity: 100%, m/z=608[M-H⁺], retention time=2.03 min.

### Example 289

2-{[3-chloro-5-(trifluoromethyl)pyridin-2-yl]oxy}-4-(2-methoxyethoxy)benzyl {[(2-oxoazepan-3-yl)amino]sulfonyl}carbamate yield: 51%, LC-MS purity: 100%, m/z=609[M-H⁺], retention time=1.80 min.

### Example 290

2-{[3-chloro-5-(trifluoromethyl)pyridin-2-yl]oxy}-4-(2-methoxyethoxy)benzyl [(2,3-dihydro-1H-inden-2-ylamino)sulfonyl]carbamate yield: 63%, LC-MS purity: 100%, m/z=614[M-H⁺], retention time=2.00 min.

### Example 291

2-{[3-chloro-5-(trifluoromethyl)pyridin-2-yl]oxy}-4-(2-methoxyethoxy)benzyl [(2,3-dihydro-1H-inden-1-ylamino)sulfonyl]carbamate yield: 55%, LC-MS purity: 100%, m/z=614[M-H⁺], retention time=2.02 min.

### Example 292

2-{[3-chloro-5-(trifluoromethyl)pyridin-2-yl]oxy}-4-(2-methoxyethoxy)benzyl [(1,2,3,4-tetrahydronaphthalen-1-ylamino)sulfonyl]carbamate yield: 56%, LC-MS purity: 100%, m/z=628[M-H⁺], retention time=2.05 min.

### Example 293

2-{[3-chloro-5-(trifluoromethyl)pyridin-2-yl]oxy}-4-(2-methoxyethoxy)benzyl {[(2-phenoxyethyl)amino]sulfonyl}carbamate yield: 70%, LC-MS purity: 95%, m/z=618[M-H⁺], retention time=1.94 min.

### Example 294

2-{[3-chloro-5-(trifluoromethyl)pyridin-2-yl]oxy}-4-(2-methoxyethoxy)benzyl {[(4-methoxybenzyl)amino]sulfonyl}carbamate yield: 66%, LC-MS purity: 95%, m/z=618[M-H⁺], retention time=1.92 min.

### Example 295

2-{[3-chloro-5-(trifluoromethyl)pyridin-2-yl]oxy}-4-(2-methoxyethoxy)benzyl ({[3-(2-oxopyrrolidin-1-yl)propyl]amino}sulfonyl)carbamate yield: 41%, LC-MS purity: 100%, m/z=623[M-H⁺], retention time=1.76 min.

### Example 296

2-{[3-chloro-5-(trifluoromethyl)pyridin-2-yl]oxy}-4-(2-methoxyethoxy)benzyl (pyrrolidin-1-ylsulfonyl)carbamate yield: 65%, LC-MS purity: 95%, m/z=552[M-H⁺], retention time=1.95 min.

### Example 297

2-{[3-chloro-5-(trifluoromethyl)pyridin-2-yl]oxy}-4-(2-methoxyethoxy)benzyl (piperidin-1-ylsulfonyl)carbamate yield: 50%, LC-MS purity: 100%, m/z=566[M-H⁺], retention time=1.99 min.

### Example 298

2-{[3-chloro-5-(trifluoromethyl)pyridin-2-yl]oxy}-4-(2-methoxyethoxy)benzyl {[(2-methoxyethyl) (methyl)amino]sulfonyl}carbamate yield: 58%, LC-MS purity: 100%, m/z=570[M-H⁺], retention time=1.89 min.

### Example 299

2-{[3-chloro-5-(trifluoromethyl)pyridin-2-yl]oxy}-4-(2-methoxyethoxy)benzyl [(4-methylpiperidin-1-yl)sulfonyl]carbamate yield: 46%, LC-MS purity: 100%, m/z=580[M-H⁺], retention time=2.05 min.

### Example 300

2-{[3-chloro-5-(trifluoromethyl)pyridin-2-yl]oxy}-4-(2-methoxyethoxy)benzyl [(dipropylamino) sulfonyl]carbamate yield: 57%, LC-MS purity: 100%, m/z=582[M-H⁺], retention time=2.15 min.

### Example 301

2-{[3-chloro-5-(trifluoromethyl)pyridin-2-yl]oxy}-4-(2-methoxyethoxy)benzyl {[ethyl(2-methoxyethyl)amino]sulfonyl}carbamate yield: 62%, LC-MS purity: 100%, m/z=584[M-H⁺], retention time=1.97 min.

### Example 302

2-{[3-chloro-5-(trifluoromethyl)pyridin-2-yl]oxy}-4-(2-methoxyethoxy)benzyl (thiomorpholin-4-ylsulfonyl) carbamate yield 49%, LC-MS purity: 97%, m/z=584[M-H⁺], retention time=1.88 min.

### Example 303

2-{[3-chloro-5-(trifluoromethyl)pyridin-2-yl]oxy}-4-(2-methoxyethoxy)benzyl {[cyclohexyl(methyl)amino]sulfonyl}carbamate yield: 61%, LC-MS purity: 100%, m/z=594[M-H⁺], retention time=2.15 min.

### Example 304

2-{[3-chloro-5-(trifluoromethyl)pyridin-2-yl]oxy}-4-(2-methoxyethoxy)benzyl {[(2R)-2-(methoxymethyl)pyrrolidin-1-yl]sulfonyl}carbamate yield: 54%, LC-MS purity: 95%, m/z=596[M-H⁺], retention time=1.95 min.

### Example 305

2-{[3-chloro-5-(trifluoromethyl)pyridin-2-yl]oxy}-4-(2-methoxyethoxy)benzyl {[(2S)-2-(methoxymethyl)pyrrolidin-1-yl]sulfonyl}carbamate yield: 61%, LC-MS purity: 95%, m/z=596[M-H⁺], retention time=1.95 min.

### Example 306

2-{[3-chloro-5-(trifluoromethyl)pyridin-2-yl]oxy}-4-(2-methoxyethoxy)benzyl {[(2R,6S)-2,6-dimethylmorpholin-4-yl]sulfonyl}carbamate yield: 51%, LC-MS purity: 95%, m/z=596[M-H⁺], retention time-1.85 min.

### Example 307

2-{[3-chloro-5-(trifluoromethyl)pyridin-2-yl]oxy}-4-(2-methoxyethoxy)benzyl (1,3-dihydro-2H-isoindol-2-ylsulfonyl)carbamate yield: 47%, LC-MS purity: 100%, m/z=600[M-H⁺], retention time=1.97 min.

### Example 308

2-{[3-chloro-5-(trifluoromethyl)pyridin-2-yl]oxy}-4-(2-methoxyethoxy)benzyl {[benzyl(methyl)amino]sulfonyl}carbamate yield: 51%, LC-MS purity: 100%, m/z=602[M-H⁺], retention time=2.03 min.

### Example 309

2-{[3-chloro-5-(trifluoromethyl)pyridin-2-yl]oxy}-4-(2-methoxyethoxy)benzyl {[4-(aminocarbonyl)piperidin-1-yl]sulfonyl}carbamate yield: 3%, LC-MS purity: 100%, m/z=609[M-H⁺], retention time=1.70 min.

### Example 310

2-{[3-chloro-5-(trifluoromethyl)pyridin-2-yl]oxy}-4-(2-methoxyethoxy)benzyl {[3-(aminocarbonyl)piperidin-1-yl]sulfonyl}carbamate yield: 30%, LC-MS purity: 100%, m/z=609[M-H⁺], retention time=1.70 min.

### Example 311

2-{[3-chloro-5-(trifluoromethyl)pyridin-2-yl]oxy}-4-(2-methoxyethoxy)benzyl {[3-(acetylamino)pyrrolidin-1-yl]sulfonyl}carbamate yield: 37%, LC-MS purity: 100%, m/z=609[M-H⁺], retention time=1.70 min.

### Example 312

2-{[3-chloro-5-(trifluoromethyl)pyridin-2-yl]oxy}-4-(2-methoxyethoxy)benzyl ({[2-(dimethylamino)ethyl]amino}sulfonyl)carbamate yield: 33%, LC-MS purity: 100%, m/z=569[M-H⁺], retention time=1.67 min.

### Example 313

2-{[3-chloro-5-(trifluoromethyl)pyridin-2-yl]oxy}-4-(2-methoxyethoxy)benzyl [(piperidin-1-ylamino)sulfonyl]carbamate yield: 5%, LC-MS purity: 90%, m/z=581[M-H⁺], retention time=1.65 min.

### Example 314

2-{[3-chloro-5-(trifluoromethyl)pyridin-2-yl]oxy}-4-(2-methoxyethoxy)benzyl ({[3-(dimethylamino)propyl]amino}sulfonyl)carbamate yield: 53%, LC-MS purity: 100%, m/z=583[M-H⁺], retention time=1.67 min.

### Example 315

2-{[3-chloro-5-(trifluoromethyl)pyridin-2-yl]oxy}-4-(2-methoxyethoxy)benzyl {[(2-pyrrolidin-1-ylethyl)amino]sulfonyl}carbamate yield: 31%, LC-MS purity: 100%, m/z=595[M-H⁺], retention time=1.70 min.

¹H-NMR (400 MHz, CDCl₃): 1.89-2.00 (4H, m), 3.07 (2H, t, J=7.0 Hz), 3.12-3.24 (4H, m), 3.26-3.33 (2H, t, J=6.9 Hz), 3.44 (3H, s), 3.72-3.75 (2H, m), 4.09-4.12 (2H, m), 4.92 (2H, s), 6.68 (1H, d, J=2.5 Hz), 6.88 (1H, dd, J=8.6, 2.5 Hz), 7.48 (1H, d, J=8.6 Hz), 7.99 (1H, d, J=2.2 Hz), 8.22 (1H, d, J=1.2 Hz).

### Example 316

2-{[3-chloro-5-(trifluoromethyl)pyridin-2-yl]oxy}-4-(2-methoxyethoxy)benzyl {[(pyridin-2-ylmethyl)amino]sulfonyl}carbamate yield: 50%, LC-MS purity: 100%, m/z=589[M-H⁺], retention time=1.77 min.

### Example 317

2-{[3-chloro-5-(trifluoromethyl)pyridin-2-yl]oxy}-4-(2-methoxyethoxy)benzyl {[(pyridin-4-ylmethyl)amino]sulfonyl}carbamate yield: 49%, LC-MS purity: 100%, m/z=589[M-H⁺], retention time=1.72 min.

### Example 318

2-{[3-chloro-5-(trifluoromethyl)pyridin-2-yl]oxy}-4-(2-methoxyethoxy)benzyl {[(2-pyridin-2-ylethyl)amino]sulfonyl}carbamate yield: 58%, LC-MS purity: 100%, m/z=603[M-H⁺], retention time=1.80 min.

### Example 319

2-{[3-chloro-5-(trifluoromethyl)pyridin-2-yl]oxy}-4-(2-methoxyethoxy)benzyl {[(2-pyridin-4-ylethyl)amino]sulfonyl}carbamate yield: 67%, LC-MS purity: 100%, m/z=603[M-H⁺], retention time=1.75 min.

### Example 320

2-{[3-chloro-5-(trifluoromethyl)pyridin-2-yl]oxy}-4-(2-methoxyethoxy)benzyl {[(2-pyridin-3-ylethyl)amino]sulfonyl}carbamate yield: 54%, LC-MS purity: 100%, m/z=603[M-H⁺], retention time=1.77 min.

¹H-NMR (400 MHz, CDCl₃) δ:2.61 (2H, d, J=3.7 Hz), 3.00-3.09 (2H, m), 3.39 (3H, s), 3.62-3.69 (2H, m), 3.93-4.00 (2H, m), 4.89 (2H, s), 5.45-5.57 (1H, m), 6.58 (1H, d, J=2.2 Hz), 6.69 (1H, d, J=8.8 Hz), 7.02 (1H, m), 7.33 (2H, d, J=8.8 Hz), 7.87 (1H, d, J=2.2 Hz), 8.14 (1H, d, J=1.0 Hz), 8.25-8.34 (2H, m).

### Example 321

2-{[3-chloro-5-(trifluoromethyl)pyridin-2-yl]oxy}-4-(2-methoxyethoxy)benzyl ({[3-(1H-imidazol-1-yl)propyl]amino}sulfonyl)carbamate yield: 41%, LC-MS purity: 100%, m/z=606 [M-H⁺], retention time=1.70 min.

### Example 322

2-{[3-chloro-5-(trifluoromethyl)pyridin-2-yl]oxy}-4-(2-methoxyethoxy)benzyl {[(2-morpholin-4-ylethyl)amino]sulfonyl}carbamate yield: 50%, LC-MS purity: 100%, m/z=611[M-H⁺], retention time=1.72 min.

### Example 323

2-{[3-chloro-5-(trifluoromethyl)pyridin-2-yl]oxy}-4-(2-methoxyethoxy)benzyl {[(2-anilinoethyl)amino]sulfonyl}carbamate yield: 49%, LC-MS purity: 100%, m/z=617[M-H⁺], retention time=1.91 min.

### Example 324

2-{[3-chloro-5-(trifluoromethyl)pyridin-2-yl]oxy}-4-(2-methoxyethoxy)benzyl [(4-methylpiperazin-1-yl) sulfonyl]carbamate yield: 20%, LC-MS purity: 100%, m/z=581 [M-H⁺], retention time=1.67 min.

### Example 325

3-[2-{[3-chloro-5-(trifluoromethyl)pyridin-2-yl]oxy}-4-(2-methoxyethoxy)phenyl]propyl [(isopropylamino)sulfonyl]carbamate yield: 51%, LC-MS purity: 100%, m/z=568[M-H⁺], retention time=1.96 min.

### Example 326

3-[2-{[3-chloro-5-(trifluoromethyl)pyridin-2-yl]oxy}-4-(2-methoxyethoxy)phenyl]propyl {[(2-hydroxyethyl)amino]sulfonyl}carbamate yield: 46%, LC-MS purity: 100%, m/z=570[M-H⁺], retention time=1.76 min.

### Example 327

3-[2-{[3-chloro-5-(trifluoromethyl)pyridin-2-yl]oxy}-4-(2-methoxyethoxy)phenyl]propyl {[(2-cyanoethyl)amino]sulfonyl}carbamate yield: 64%, LC-MS purity: 95%, m/z=579[M-H⁺], retention time=1.79 min.

### Example 328

3-[2-{[3-chloro-5-(trifluoromethyl)pyridin-2-yl]oxy}-4-(2-methoxyethoxy)phenyl]propyl [(tert-butylamino)sulfonyl]carbamate yield: 52%, LC-MS purity: 100%, m/z=582[M-H⁺], retention time=2.02 min.

¹H-NMR (400 MHz, CDCl₃) δ:1.30 (9H, s), 1.86-1.94 (2H, m), 2.52 (2H, t, J=7.5 Hz), 3.43 (3H, s), 3.69-3.75 (2H, m), 4.05-4.13 (4H, m), 5.08 (1H, s), 6.67 (1H, d, J=2.5 Hz), 6.81 (1H, dd, J=8.3, 2.5 Hz), 7.17 (1H, d, J=8.6 Hz), 7.99 (1H, d, J=2.2 Hz), 8.26 (1H, s)

### Example 329

3-[2-{[3-chloro-5-(trifluoromethyl)pyridin-2-yl]oxy}-4-(2-methoxyethoxy)phenyl]propyl {[(3-hydroxypropyl)amino]sulfonyl}carbamate yield: 47%, LC-MS purity: 100%, m/z=584[M-H⁺], retention time=1.78 min.

### Example 330

3-[2-{[3-chloro-5-(trifluoromethyl)pyridin-2-yl]oxy}-4-(2-methoxyethoxy)phenyl]propyl {[(2-methoxyethyl)amino]sulfonyl}carbamate yield: 65%, LC-MS purity: 90%, m/z=584[M-H⁺, retention time=1.85 min.

### Example 331

3-[2-{[3-chloro-5-(trifluoromethyl)pyridin-2-yl]oxy}-4-(2-methoxyethoxy)phenyl]propyl [(cyclopentylamino)sulfonyl]carbamate yield: 58%, LC-MS purity: 100%, m/z=594[M-H⁺], retention time=2.02 min.

### Example 332

3 [2-{[3-chloro-5-(trifluoromethyl)pyridin-2-yl]oxy}-4-(2-methoxyethoxy)phenyl]propyl {[(1-ethylpropyl)amino]sulfonyl}carbamate yield: 62%, LC-MS purity: 100%, m/z=596[M-H⁺], retention time=2.08 min.

### Example 333

3-[2-{[3-chloro-5-(trifluoromethyl)pyridin-2-yl]oxy}-4-(2-methoxyethoxy)phenyl]propyl {[(3-methylbutyl)amino]sulfonyl}carbamate yield: 59%, LC-MS purity: 100%, m/z=596[M-H⁺], retention time=2.06 min.

### Example 334

3-[2-{[3-chloro-5-(trifluoromethyl)pyridin-2-yl]oxy}-4-(2-methoxyethoxy)phenyl]propyl {[(1,2-dimethylpropyl)amino]sulfonyl}carbamate yield: 58%, LC-MS purity: 100%, m/z=596[M-H⁺], retention time=2.07 min.

### Example 335

3-[2-{[3-chloro-5-(trifluoromethyl)pyridin-2-yl]oxy}-4-(2-methoxyethoxy)phenyl]propyl {[(1-methylbutyl)amino]sulfonyl}carbamate yield: 59%, LC-MS purity: 100%, m/z=596[M-H⁺], retention time=2.08 min.

### Example 336

3-[2-{[3-chloro-5-(trifluoromethyl)pyridin-2-yl]oxy}-4-(2-methoxyethoxy)phenyl]propyl {[(4-hydroxybutyl)amino]sulfonyl}carbamate yield: 45%, LC-MS purity: 100%, m/z=598[M-H⁺], retention time=1.80 min.

### Example 337

3-[2-{[3-chloro-5-(trifluoromethyl)pyridin-2-yl]oxy}-4-(2-methoxyethoxy)phenyl]propyl [(cyclohexylamino)sulfonyl]carbamate yield: 60%, LC-MS purity: 100%, m/z=608[M-H⁺], retention time=2.07 min.

### Example 338

3-[2-{[3-chloro-5-(trifluoromethyl)pyridin-2-yl]oxy}-4-(2-methoxyethoxy)phenyl]propyl {[(tetrahydrofuran-2-ylmethyl)amino]sulfonyl}carbamate yield: 66%, LC-MS purity: 100%, m/z=610[M-H⁺], retention time=1.88 min.

### Example 339

3-[2-{[3-chloro-5-(trifluoromethyl)pyridin-2-yl]oxy}-4-(2-methoxyethoxy)phenyl]propyl {[(1,3-dimethylbutyl)amino]sulfonyl}carbamate yield: 57%, LC-MS purity: 100%, m/z=610[M-H⁺], retention time=2.13 min.

### Example 340

3-[2-{[3-chloro-5-(trifluoromethyl)pyridin-2-yl]oxy}-4-(2-methoxyethoxy)phenyl]propyl ({[2-(acetylamino)ethyl]amino}sulfonyl)carbamate yield: 25%, LC-MS purity: 100%, m/z=611[M-H⁺], retention time=1.74 min.

### Example 341

3-[2-{[3-chloro-5-(trifluoromethyl)pyridin-2-yl]oxy}-4-(2-methoxyethoxy)phenyl]propyl {[(2-isopropoxyethyl)amino]sulfonyl}carbamate yield: 56%, LC-MS purity: 100%, m/z=612[M-H⁺], retention time=1.94 min.

### Example 342

3-[2-{[3-chloro-5-(trifluoromethyl)pyridin-2-yl]oxy}-4-(2-methoxyethoxy)phenyl]propyl ({[1-(methoxymethyl)propyl]amino}sulfonyl)carbamate yield: 54%, LC-MS purity: 95%, m/z=612[M-H⁺], retention time=1.98 min.

### Example 343

3-[2-{[3-chloro-5-(trifluoromethyl)pyridin-2-yl]oxy}-4-(2-methoxyethoxy)phenyl]propyl {[(3-ethoxypropyl)amino]sulfonyl}carbamate yield: 57%, LC-MS purity: 100%, m/z=612[M-H⁺], retention time=1.94 min.

### Example 344

3-[2-{[3-chloro-5-(trifluoromethyl)pyridin-2-yl]oxy}-4-(2-methoxyethoxy)phenyl]propyl ({[3-(methylthio)propyl]amino}sulfonyl)carbamate yield: 59%, LC-MS purity: 100%, m/z=614[M-H⁺], retention time=1.96 min.

### Example 345

3-[2-{[3-chloro-5-(trifluoromethyl)pyridin-2-yl]oxy}-4-(2-methoxyethoxy)phenyl]propyl [(benzylamino)sulfonyl]carbamate yield: 57%, LC-MS purity: 100%, m/z=616[M-H⁺], retention time=1.99 min.

### Example 346

3-[2-{[3-chloro-5-(trifluoromethyl)pyridin-2-yl]oxy}-4-(2-methoxyethoxy)phenyl]propyl {[(2-amino-2-oxoethyl)amino]sulfonyl}carbamate yield: 16%, LC-MS purity: 100%, m/z=583[M-H⁺], retention time=1.68 min.

### Example 347

3-[2-{[3-chloro-5-(trifluoromethyl)pyridin-2-yl]oxy}-4-(2-methoxyethoxy)phenyl]propyl {[(cyclohexylmethyl)amino]sulfonyl}carbamate yield: 61%, LC-MS purity: 100%, m/z=622[M-H⁺], retention time=2.13 min.

### Example 348

3-[2-{[3-chloro-5-(trifluoromethyl)pyridin-2-yl]oxy}-4-(2-methoxyethoxy)phenyl]propyl [(cycloheptylamino)sulfonyl]carbamate yield: 48%, LC-MS purity: 100%, m/z=622[M-H⁺], retention time=2.13 min.

¹H-NMR (400 MHz, CDCl₃) δ:1.34-1.60 (10H, m), 1.87-1.96 (4H, m), 2.53 (2H, t, J=7.3 Hz), 3.43 (3H, s), 3.38-3.46 (1H, m), 3.70-3.75 (2H, m), 4.05-4.14 (4H, m), 5.00-5.09 (1H, m), 6.68 (1H, d, J=2.2 Hz), 6.82 (1H, dd, J=8.6, 2.5 Hz), 7.18 (1H, d, J=8.6 Hz), 8.00 (1H, d, J=2.0 Hz), 8.26 (1H, s).

### Example 349

3-[2-{[3-chloro-5-(trifluoromethyl)pyridin-2-yl]oxy}-4-(2-methoxyethoxy)phenyl]propyl [(cycloheptylamino)sulfonyl]carbamate yield: 58%, LC-MS purity: 100%, m/z=622[M-H⁺], retention time=2.13 min.

### Example 350

3-[2-{[3-chloro-5-(trifluoromethyl)pyridin-2-yl]oxy}-4-(2-methoxyethoxy)phenyl]propyl {[(3-isopropoxypropyl)amino]sulfonyl}carbamate yield: 53%, LC-MS purity: 100%, m/z=626[M-H⁺], retention time=2.00 min.

### Example 351

3-[2-{[3-chloro-5-(trifluoromethyl)pyridin-2-yl]oxy}-4-(2-methoxyethoxy)phenyl]propyl {[(2-phenylethyl)amino]sulfonyl}carbamate yield: 54%, LC-MS purity: 100%, m/z=630[M-H⁺], retention time=2.03 min.

### Example 352

3-[2-{[3-chloro-5-(trifluoromethyl)pyridin-2-yl]oxy}-4-(2-methoxyethoxy)phenyl]propyl ({[2-(2-thienyl)ethyl]amino}sulfonyl)carbamate yield: 47%, LC-MS purity: 100%, m/z=636[M-H⁺], retention time=2.00 min.

### Example 353

3-[2-{[3-chloro-5-(trifluoromethyl)pyridin-2-yl]oxy}-4-(2-methoxyethoxy)phenyl]propyl {[(2-oxoazepan-3-yl)amino]sulfonyl}carbamate yield: 48%, LC-MS purity: 90%, m/z=637[M-H⁺], retention time=1.81 min.

### Example 354

3-[2-{[3-chloro-5-(trifluoromethyl)pyridin-2-yl]oxy}-4-(2-methoxyethoxy)phenyl]propyl [(2,3-dihydro-1H-inden-2-ylamino)sulfonyl]carbamate yield: 54%, LC-MS purity: 100%, m/z=642[M-H⁺], retention time=2.04 min.

### Example 355

3-[2-{[3-chloro-5-(trifluoromethyl)pyridin-2-yl]oxy}-4-(2-methoxyethoxy)phenyl]propyl [(2,3-dihydro-1H-inden-1-ylamino)sulfonyl]carbamate yield: 51%, LC-MS purity: 100%, m/z=642[M-H⁺], retention time=2.08 min.

### Example 356

3-[2-{[3-chloro-5-(trifluoromethyl)pyridin-2-yl]oxy}-4-(2-methoxyethoxy)phenyl]propyl [(1,2,3,4-tetrahydronaphthalen-1-ylamino)sulfonyl]carbamate yield: 51%, LC-MS purity: 100%, m/z=656[M-H⁺], retention time=2.11 min.

### Example 357

3-[2-{[3-chloro-5-(trifluoromethyl)pyridin-2-yl]oxy}-4-(2-methoxyethoxy)phenyl]propyl {[(2-phenoxyethyl)amino]sulfonyl}carbamate yield: 47%, LC-MS purity: 100%, m/z=646[M-H⁺], retention time=2.00 min.

¹H-NMR (400 MHz, CDCl₃) δ:1.77-1.89 (2H, m), 2.50 (2H, t, J=7.2 Hz), 3.42 (3H, s), 3.38-3.47 (2H, m), 3.65-3.73 (2H, m), 3.98-4.08 (6H, m), 5.53-5.65 (1H, m), 6.66 (1H, d, J=2.5 Hz), 6.76-6.86 (3H, m), 6.92 (1H, t, J=7.1 Hz), 7.13 (1H, d, J=8.6 Hz), 7.23 (2H, t, J=7.8 Hz), 7.98 (1H, d, J=2.0 Hz), 8.25 (1H, s).

### Example 358

3-[2-{[3-chloro-5-(trifluoromethyl)pyridin-2-yl]oxy}-4-(2-methoxyethoxy)phenyl]propyl {[(4-methoxybenzyl)amino]sulfonyl}carbamate yield: 48%, LC-MS purity: 100%, m/z=646[M-H⁺], retention time=1.98 min.

### Example 359

3-[2-{[3-chloro-5-(trifluoromethyl)pyridin-2-yl]oxy}-4-(2-methoxyethoxy)phenyl]propyl ({[3-(2-oxopyrrolidin-1-yl)propyl]amino}sulfonyl)carbamate yield: 40%, LC-MS purity: 82%, m/z=651[M-H⁺], retention time=1.80 min.

### Example 360

3-[2-{[3-chloro-5-(trifluoromethyl)pyridin-2-yl]oxy}-4-(2-methoxyethoxy)phenyl]propyl (pyrrolidin-1-ylsulfonyl)carbamate yield: 57%, LC-MS purity: 100%, m/z=580[M-H⁺], retention time=2.00 min.

### Example 361

3-[2-{[3-chloro-5-(trifluoromethyl)pyridin-2-yl]oxy}-4-(2-methoxyethoxy)phenyl]propyl (piperidin-1-ylsulfonyl)carbamate yield: 59%, LC-MS purity: 100%, m/z=594[M-H⁺], retention time=2.05 min.

### Example 362

3-[2-{[3-chloro-5-(trifluoromethyl)pyridin-2-yl]oxy}-4-(2-methoxyethoxy)phenyl]propyl {[(2-methoxyethyl)(methyl)amino]sulfonyl}carbamate yield: 59%, LC-MS purity: 100%, m/z=598 [M-H⁺], retention time=1.95 min.

### Example 363

3-[2-{[3-chloro-5-(trifluoromethyl)pyridin-2-yl]oxy}-4-(2-methoxyethoxy)phenyl]propyl [(4-methylpiperidin-1-yl)sulfonyl]carbamate yield: 46%, LC-MS purity: 94%, m/z=608[M-H⁺], retention time=2.11 min.

### Example 364

3-[2-{[3-chloro-5-(trifluoromethyl)pyridin-2-yl]oxy}-4-(2-methoxyethoxy)phenyl]propyl [(dipropylamino)sulfonyl]carbamate yield: 50%, LC-MS purity: 100%, m/z=610-[M-H⁺], retention time=2.19 min.

### Example 365

3-[2-{[3-chloro-5-(trifluoromethyl)pyridin-2-yl]oxy}-4-(2-methoxyethoxy)phenyl]propyl {[ethyl(2-methoxyethyl)amino]sulfonyl}carbamate yield: 50%, LC-MS purity: 100%, m/z=612[M-H⁺], retention time=2.02 min.

### Example 366

3-[2-{[3-chloro-5-(trifluoromethyl)pyridin-2-yl]oxy}-4-(2-methoxyethoxy)phenyl]propyl (thiomorpholin-4-ylsulfonyl)carbamate yield: 59%, LC-MS purity: 100%, m/z=612[M-H⁺], retention time=1.94 min.

### Example 367

3-[2-{[3-chloro-5-(trifluoromethyl)pyridin-2-yl]oxy}-4-(2-methoxyethoxy)phenyl]propyl {[cyclohexyl(methyl)amino]sulfonyl}carbamate yield: 60%, LC-MS purity: 100%, m/z=622[M-H⁺], retention time=2.19 min.

### Example 368

3-[2-{[3-chloro-5-(trifluoromethyl)pyridin-2-yl]oxy}-4-(2-methoxyethoxy)phenyl]propyl {[(2R)-2-(methoxymethyl)pyrrolidin-1-yl]sulfonyl}carbamate yield: 52%, LC-MS purity: 100%, m/z=624[M-H⁺], retention time=2.01 min.

### Example 369

3-[2-{[3-chloro-5-(trifluoromethyl)pyridin-2-yl]oxy}-4-(2-methoxyethoxy)phenyl]propyl {[(2S)-2-(methoxymethyl)pyrrolidin-1-yl]sulfonyl}carbamate yield: 59%, LC-MS purity: 100%, m/z=624[M-H⁺], retention time=2.01 min.

### Example 370

3-[2-{[3-chloro-5-(trifluoromethyl)pyridin-2-yl]oxy}-4-(2-methoxyethoxy)phenyl]propyl{[(2R,6S)-2,6-dimethylmorpholin-4-yl]sulfonyl}carbamate yield: 55%, LC-MS purity: 100%, m/z=624[M-H⁺], retention time=1.92 min.

### Example 371

3-[2-{[3-chloro-5-(trifluoromethyl)pyridin-2-yl]oxy}-4-(2-methoxyethoxy)phenyl]propyl (1,3-dihydro-2H-isoindol-2-ylsulfonyl)carbamate yield: 56%, LC-MS purity: 100%, m/z=628[M-H⁺], retention time=2.04 min.

### Example 372

3-[2-{[3-chloro-5-(trifluoromethyl)pyridin-2-yl]oxy}-4-(2-methoxyethoxy)phenyl]propyl {[benzyl(methyl)amino]sulfonyl}carbamate yield: 57%, LC-MS purity: 106%, m/z=630-[M-H⁺], retention time=2.10 min.

### Example 373

3-[2-{[3-chloro-5-(trifluoromethyl)pyridin-2-yl]oxy}-4-(2-methoxyethoxy)phenyl]propyl {[4-(aminocarbonyl)piperidin-1-yl]sulfonyl}carbamate yield: 6%, LC-MS purity: 100%, m/z=637[M-H⁺], retention time=1.76 min.

### Example 374

3-[2-{[3-chloro-5-(trifluoromethyl)pyridin-2-yl]oxy}-4-(2-methoxyethoxy)phenyl]propyl {[3-(aminocarbonyl)piperidin-1-yl]sulfonyl}carbamate yield: 47%, LC-MS purity: 100%, m/z=637[M-H⁺], retention time=1.77 min.

### Example 375

3-[2-{[3-chloro-5-(trifluoromethyl)pyridin-2-yl]oxy}-4-(2-methoxyethoxy)phenyl]propyl {[3-(acetylamino)pyrrolidin-1-yl]sulfonyl}carbamate yield: 48%, LC-MS purity: 100%, m/z=637[M-H⁺], retention time=1.77 min.

### Example 376

3-[2-{[3-chloro-5-(trifluoromethyl)pyridin-2-yl]oxy}-4-(2-methoxyethoxy)phenyl]propyl ({[2-(dimethylamino)ethyl]amino}sulfonyl)carbamate yield: 49%, LC-MS purity: 100%, m/z=597[M-H⁺], retention time=1.72 min.

### Example 377

3-[2-{[3-chloro-5-(trifluoromethyl)pyridin-2-yl]oxy}-4-(2-methoxyethoxy)phenyl]propyl ({[3-(dimethylamino)propyl]amino}sulfonyl)carbamate yield: 39%, LC-MS purity: 100%, m/z=611[M-H⁺], retention time=1.72 min.

### Example 378

3-[2-{[3-chloro-5-(trifluoromethyl)pyridin-2-yl]oxy}-4-(2-methoxyethoxy)phenyl]propyl {[(2-pyrrolidin-1-ylethyl)amino]sulfonyl}carbamate yield: 48%, LC-MS purity: 100%, m/z=623[M-H⁺], retention time=1.74 min.

### Example 379

3-[2-{[3-chloro-5-(trifluoromethyl)pyridin-2-yl]oxy}-4-(2-methoxyethoxy)phenyl]propyl {[(pyridin-2-ylmethyl)amino]sulfonyl}carbamate yield: 43%, LC-MS purity: 100%, m/z=617[M-H⁺], retention time=1.83 min.

### Example 380

3-[2-{[3-chloro-5-(trifluoromethyl)pyridin-2-yl]oxy}-4-(2-methoxyethoxy)phenyl]propyl {[(pyridin-4-ylmethyl)amino]sulfonyl}carbamate yield: 48%, LC-MS purity: 100%, m/z=617[M-H⁺], retention time=1.78 min.

### Example 381

3-[2-{[3-chloro-5-(trifluoromethyl)pyridin-2-yl]oxy}-4-(2-methoxyethoxy)phenyl]propyl {[(2-pyridin-2-ylethyl)amino]sulfonyl}carbamate yield: 49%, LC-MS purity: 100%, m/z=631 [M-H⁺], retention time=1.87 min.

### Example 382

3-[2-{[3-chloro-5-(trifluoromethyl)pyridin-2-yl]oxy}-4-(2-methoxyethoxy)phenyl]propyl {[(2-pyridin-4-ylethyl)amino]sulfonyl}carbamate yield: 48%, LC-MS purity: 100%, m/z=631[M-H⁺], retention time=1.81 min.

### Example 383

3-[2-{[3-chloro-5-(trifluoromethyl)pyridin-2-yl]oxy}-4-(2-methoxyethoxy)phenyl]propyl {[(2-pyridin-3-ylethyl)amino]sulfonyl}carbamate yield: 43%, LC-MS purity: 100%, m/z=631[M-H⁺], retention time=1.83 min.

### Example 384

3-[2-{[3-chloro-5-(trifluoromethyl)pyridin-2-yl]oxy}-4-(2-methoxyethoxy)phenyl]propyl ({[3-(1H-imidazol-1-yl)propyl]amino}sulfonyl)carbamate yield: 39%, LC-MS purity: 100%, m/z=634[M-H⁺], retention time=1.76 min.

### Example 385

3-[2-{[3-chloro-5-(trifluoromethyl)pyridin-2-yl]oxy}-4-(2-methoxyethoxy)phenyl]propyl {[(2-morpholin-4-ylethyl)amino]sulfonyl}carbamate yield: 46%, LC-MS purity: 100%, m/z=639[M-H⁺], retention time=1.77 min.

### Example 386

3-[2-{[3-chloro-5-(trifluoromethyl)pyridin-2-yl]oxy}-4-(2-methoxyethoxy)phenyl]propyl {[(2-anilinoethyl)amino]sulfonyl}carbamate yield: 47%, LC-MS purity: 89%, m/z=645[M-H⁺], retention time=1.97 min.

### Example 387

3-[2-{[3-chloro-5-(trifluoromethyl)pyridin-2-yl]oxy}-4-(2-methoxyethoxy)phenyl]propyl [(4-methylpiperazin-1-yl)sulfonyl]carbamate yield: 36%, LC-MS purity: 100%, m/z=609[M-H⁺], retention time=1.72 min.

### Example 388

3-[2-(cyclohexyloxy)-4-(2-methoxyethoxy)phenyl]propyl [(pentylamino)sulfonyl]carbamate yield: 21%, LC-MS purity: 100%, m/z=499 [M-H⁺], retention time=2.11 min.

### Example 389

3-[2-(cyclohexyloxy)-4-(2-methoxyethoxy)phenyl]propyl {[(2-isopropoxyethyl)amino]sulfonyl}carbamate yield: 13%, LC-MS purity: 100%, m/z=515[M-H⁺], retention time=2.06 min.

### Example 390

3-[2-(cyclohexyloxy)-4-(2-methoxyethoxy)phenyl]propyl {[(2-phenylethyl)amino]sulfonyl}carbamate yield: 16%, LC-MS purity: 100%, m/z=533[M-H⁺], retention time=2.14 min.

### Example 391

3-[2-(cyclohexyloxy)-4-(2-methoxyethoxy)phenyl]propyl ({[2-(2-thienyl)ethyl]amino}sulfonyl)carbamate yield: 12%, LC-MS purity: 100%, m/z=539[M-H⁺], retention time=2.13 min.

### Example 392

3-[2-(cyclohexyloxy)-4-(2-methoxyethoxy)phenyl]propyl {[(2-phenoxyethyl)amino]sulfonyl}carbamate yield: 19%, LC-MS purity: 87%, m/z=549[M-H⁺], retention time=2.09 min.

### Example 393

3-[2-(cyclohexyloxy)-4-(2-methoxyethoxy)phenyl]propyl ({[2-(2-methoxyphenyl)ethyl]amino}sulfonyl)carbamate yield: 18%, LC-MS purity: 100%, m/z=563[M-H⁺], retention time=2.10 min.

### Example 394

3-[2-(cyclohexyloxy)-4-(2-methoxyethoxy)phenyl]propyl {[(2,2-diphenylethyl)amino]sulfonyl}carbamate yield: 23%, LC-MS purity: 100%, m/z=609[M-H⁺], retention time-2.26 min.

### Example 395

3-[2-(cyclohexyloxy)-4-(2-methoxyethoxy)phenyl]propyl {[(1,2-diphenylethyl)amino]sulfonyl}carbamate yield: 30%, LC-MS purity: 100%, m/z=609[M-H⁺], retention time=2.23 min.

¹H-NMR (400 MHz, CDCl₃) δ:1.30-1.42 (2H, m), 1.45-1.68 (4H, m), 1.69-1.79 (4H, m), 1.88-1.97 (2H, m), 2.45-2.52 (2H, m), 3.01-3.09 (1H, m), 3.12-3.18 (1H, m), 3.44 (3H, s), 3.71-3.76 (2H, m), 3.79-3.84 (1H, m), 3.86-3.91 (1H, m), 4.05-4.11 (2H, m), 4.18-4.25 (1H, m), 4.59-4.67 (1H, m), 5.59-5.67 (1H, m), 6.34-6.40 (1H, m), 6.50 (3H, d, J=2.2 Hz), 6.92 (1H, d, J=8.3 Hz), 6.97-7.08 (3H, m), 7.14-7.24 (7H, m).

### Example 396

3-[2-(cyclohexyloxy)-4-(2-methoxyethoxy)phenyl]propyl {[(pyridin-2-ylmethyl)amino]sulfonyl}carbamate yield: 23%, LC-MS purity: 81%, m/z=520[M-H⁺], retention time=2.03 min.

### Example 397

3-[2-(cyclohexyloxy)-4-(2-methoxyethoxy)phenyl]propyl {[(2-pyridin-2-ylethyl)amino]sulfonyl}carbamate yield: 27%, LC-MS purity: 100%, m/z=534[M-H⁺], retention time=2.04 min.

### Example 398

3-[2-(cyclohexyloxy)-4-(2-methoxyethoxy)phenyl]propyl ({[(5-methylpyrazin-2-yl)methyl]amino}sulfonyl)carbamate yield: 28%, LC-MS purity: 100%, m/z=535[M-H⁺], retention time=1.99 min.

### Example 399

3-[2-(cyclohexyloxy)-4-(2-methoxyethoxy)phenyl]propyl {[(3-methoxypropyl)amino]sulfonyl}carbamate yield: 20%, LC-MS purity: 100%, m/z=501[M-H⁺], retention time=2.08 min.

### Example 400

3-[2-(cyclopropylmethoxy)-4-(2-methoxyethoxy)phenyl]propyl [(pentylamino)sulfonyl]carbamate yield: 57%, LC-MS purity: 88%, m/z=471[M-H⁺], retention time=2.05 min.

### Example 401

3-[2-(cyclopropylmethoxy)-4-(2-methoxyethoxy)phenyl]propyl {[(2-isopropoxyethyl)amino]sulfonyl}carbamate yield: 37%, LC-MS purity: 100%, m/z=487[M-H⁺], retention time=2.04 min.

### Example 402

3-[2-(cyclopropylmethoxy)-4-(2-methoxyethoxy)phenyl]propyl {[(2-phenylethyl)amino]sulfonyl}carbamate yield: 36%, LC-MS purity: 100%, m/z=505[M-H⁺], retention time=2.05 min.

### Example 403

3-[2-(cyclopropylmethoxy)-4-(2-methoxyethoxy)phenyl]propyl ({[2-(2-thienyl)ethyl]amino}sulfonyl)carbamate yield: 50%, LC-MS purity: 100%, m/z=511[M-H⁺], retention time=2.07 min.

### Example 404

3-[2-(cyclopropylmethoxy)-4-(2-methoxyethoxy)phenyl]propyl {[(2-phenoxyethyl)amino]sulfonyl}carbamate yield: 48%, LC-MS purity: 89%, m/z=521[M-H⁺], retention time=2.04 min.

### Example 405

3-[2-(cyclopropylmethoxy)-4-(2-methoxyethoxy)phenyl]propyl ({[2-(2-methoxyphenyl)ethyl]amino}sulfonyl)carbamate yield: 46%, LC-MS purity: 100%, m/z=535[M-H⁺], retention time=2.06 min.

### Example 406

3-[2-(cyclopropylmethoxy)-4-(2-methoxyethoxy)phenyl]propyl {[(2,2-diphenylethyl)amino]sulfonyl}carbamate yield: 70%, LC-MS purity: 100%, m/z=581[M-H⁺], retention time=2.12 min.

¹H-NMR (400 MHz, CDCl₃) δ:0.23-0.31 (2H, m), 0.49-0.58 (2H, m), 1.17-1.28 (1H, m), 1.78-1.90 (2H, m), 2.59 (2H, t, J=7.3 Hz), 3.43 (3H, s), 3.63 (2H, d, J=7.8 Hz), 3.69-3.76 (4H, m), 3.97-4.07 (4H, m), 4.20 (1H, t, J=7.8 Hz), 5.03 (1H, s), 6.36 (1H, d, J=8.3 Hz), 6.44 (1H, d, J=1.7 Hz), 6.94 (1H, d, J=8.1 Hz), 7.11-7.25 (1H, m).

### Example 407

3-[2-(cyclopropylmethoxy)-4-(2-methoxyethoxy)phenyl]propyl {[(1,2-diphenylethyl)amino]sulfonyl}carbamate yield: 44%, LC-MS purity: 100%, m/z=581[M-H⁺], retention time=2.12 min.

### Example 408

3-[2-(cyclopropylmethoxy)-4-(2-methoxyethoxy)phenyl]propyl {[(pyridin-2-ylmethyl)amino]sulfonyl}carbamate yield: 37%, LC-MS purity: 100%, m/z=492[M-H⁺], retention time=1.97 min.

### Example 409

3-[2-(cyclopropylmethoxy)-4-(2-methoxyethoxy)phenyl]propyl {[(2-pyridin-2-ylethyl)amino]sulfonyl}carbamate yield: 52%, LC-MS purity: 100%, m/z=506[M-H⁺], retention time=2.01 min.

### Example 410

3-[2-(cyclopropylmethoxy)-4-(2-methoxyethoxy)phenyl]propyl ({[(5-methylpyrazin-2-yl)methyl]amino}sulfonyl)carbamate yield: 68%, LC-MS purity: 100%, m/z=507[M-H⁺], retention time=1.93 min.

### Example 411

3-[2-(cyclopropylmethoxy)-4-(2-methoxyethoxy)phenyl]propyl {[(3-methoxypropyl)amino]sulfonyl}carbamate yield: 43%, LC-MS purity: 100%, m/z=473[M-H⁺], retention time=2.01 min.

### Example 412

3-[4-(2-methoxyethoxy)-2-(tetrahydrofuran-2-ylmethoxy)phenyl]propyl [(pentylamino)sulfonyl]carbamate yield: 51%, LC-MS purity: 81%, m/z=501[M-H⁺], retention time=2.08 min.

### Example 413

3-[4-(2-methoxyethoxy)-2-(tetrahydrofuran-2-ylmethoxy)phenyl]propyl {[(2-isopropoxyethyl)amino]sulfonyl}carbamate yield: 36%, LC-MS purity: 100%, m/z=517[M-H⁺], retention time=2.01 min.

### Example 414

3-[4-(2-methoxyethoxy)-2-(tetrahydrofuran-2-ylmethoxy)phenyl]propyl {[(2-phenylethyl)amino]sulfonyl}carbamate yield: 45%, LC-MS purity: 100%, m/z=535[M-H⁺], retention time=2.01 min.

### Example 415

3-[4-(2-methoxyethoxy)-2-(tetrahydrofuran-2-ylmethoxy)phenyl]propyl ({[2-(2-thienyl)ethyl]amino}sulfonyl)carbamate yield: 46%, LC-MS purity: 100%, m/z=541[M-H⁺], retention time=1.99 min.

### Example 416

3-[4-(2-methoxyethoxy)-2-(tetrahydrofuran-2-ylmethoxy)phenyl]propyl {[(2-phenoxyethyl)amino]sulfonyl}carbamate yield: 54%, LC-MS purity: 89%, m/z=551[M-H⁺], retention time=2.02 min.

### Example 417

3-[4-(2-methoxyethoxy)-2-(tetrahydrofuran-2-ylmethoxy)phenyl]propyl ({[2-(2-methoxyphenyl)ethyl]amino}sulfonyl)carbamate yield: 49%, LC-MS purity: 100%, m/z=565[M-H⁺], retention time=2.03 min.

### Example 418

3-[4-(2-methoxyethoxy)-2-(tetrahydrofuran-2-ylmethoxy)phenyl]propyl {[(2,2-diphenylethyl)amino]sulfonyl}carbamate yield: 41%, LC-MS purity: 100%, m/z=611[M-H⁺], retention time=2.07 min.

### Example 419

3-[4-(2-methoxyethoxy)-2-(tetrahydrofuran-2-ylmethoxy)phenyl]propyl {[(1,2-diphenylethyl)amino]sulfonyl}carbamate yield: 41%, LC-MS purity: 100%, m/z=611[M-H⁺], retention time=2.09 min.

### Example 420

3-[4-(2-methoxyethoxy)-2-(tetrahydrofuran-2-ylmethoxy)phenyl]propyl {[(pyridin-2-ylmethyl)amino]sulfonyl}carbamate yield: 17%, LC-MS purity: 88%, m/z=522[M-H⁺], retention time=1.96 min.

### Example 421

3-[4-(2-methoxyethoxy)-2-(tetrahydrofuran-2-ylmethoxy)phenyl]propyl {[(2-pyridin-2-ylethyl)amino]sulfonyl}carbamate yield: 17%, LC-MS purity: 100%, m/z=536[M-H⁺], retention time=1.98 min.

### Example 422

3-[4-(2-methoxyethoxy)-2-(tetrahydrofuran-2-ylmethoxy)phenyl]propyl ({[(5-methylpyrazin-2-yl)methyl]amino}sulfonyl)carbamate yield: 18%, LC-MS purity: 100%, m/z=537[M-H⁺], retention time=1.86 min.

### Example 423

3-[4-(2-methoxyethoxy)-2-(tetrahydrofuran-2-ylmethoxy)phenyl]propyl {[(3-methoxypropyl)amino]sulfonyl}carbamate yield: 21%, LC-MS purity: 100%, m/z=503[M-H⁺], retention time=1.95 min.

### Example 424

To a solution of (2E)-3-{2-{[3-chloro-5-(trifluoromethyl)pyridin-2-yl]oxy}-4-[2-(cyclopropyloxy)ethoxy]phenyl}acrylic acid (1.73 g) in acetonitrile (30 ml) were added 1-ethyl-3-(3-dimethylaminopropyl)carbodiimide hydrochloride (1.49 g), 4-dimethylaminopyridine (0.95 g) and pentane-1-sulfonamide (592 mg), and the mixture was stirred overnight at room temperature. 1N Hydrochloric acid was added to the reaction mixture, and the mixture was diluted with ethyl acetate. The organic layer was washed with 1N hydrochloric acid, and saturated brine, dried (MgSO₄), filtrated and concentrated. The obtained residue was subjected to silica gel column chromatography, and eluted with ethyl acetate-hexane (1:19-45:55, v/v) to give a white solid. Recrystallization from ethyl acetate-hexane gave (2E)-3-{2-{[3-chloro-5-(trifluoromethyl)pyridin-2-yl]oxy}-4-[2-(cyclopropyloxy)ethoxy]phenyl}-N-(pentylsulfonyl)acrylamide (952 mg, yield: 42%) as white crystals. melting point 104.6-106.5° C.

### Example 425

To a solution of ethyl 3-{2-{[3-chloro-5-(trifluoromethyl)pyridin-2-yl]oxy}-4-[2-(2-methyl-1,3-dioxolan-2-yl)ethoxy]phenyl}propanoate (0.77 g) in tetrahydrofuran (4 ml) and ethanol (4 ml) was added a 1N aqueous sodium hydroxide solution (4 ml), and the mixture was stirred at 50° C. for 30 min. After allowing to cool to room temperature, 1N hydrochloric acid (4 ml) was added to the reaction mixture, and the mixture was diluted with ethyl acetate. The organic layer was washed with water and saturated brine, dried (MgSO₄), filtrated and concentrated to give a yellow oil.

To a solution of the obtained oil in acetonitrile (10 ml) were added 1-ethyl-3-(3-dimethylaminopropyl)carbodiimide hydrochloride (588 mg), 4-dimethylaminopyridine (283 mg) and pentane-1-sulfonamide (233 mg), and the mixture was stirred overnight at room temperature. A saturated aqueous ammonium chloride solution was added to the reaction mixture, and the mixture was diluted with ethyl acetate. The organic layer was washed with a saturated aqueous ammonium chloride solution, and saturated brine, dried (MgSO₄), filtrated and concentrated. The obtained residue was subjected to silica gel column chromatography, and eluted with ethyl acetate-hexane (1:9′-55:45, v/v) to give a white solid. Recrystallization from ethyl acetate-hexane gave 3-{2-{[3-chloro-5-(trifluoromethyl)pyridin-2-yl]oxy}-4-[2-(2-methyl-1,3-dioxolan-2-yl)ethoxy]phenyl}-N-(pentylsulfonyl)propanamide (361 mg, yield: 40%) as white crystals. melting point 96.5-97.5° C.

### Example 426

To a solution of 3-[2-[(3,5-dichloropyridin-2-yl)oxy]-4-(2-methoxyethoxy)phenyl]propan-1-ol (274 mg) in toluene (8 ml) under ice-cooling chlorosulfonylisocyanate (109 mg) was added, and the mixture was stirred for 30 min. Pyridine (175 mg) was added to the reaction mixture and the mixture was stirred for 1 hr. A 28% ammonia solution (268 mg) was added, and the mixture was stirred at room temperature for 1.5 hr. Water was added to the reaction mixture and the mixture was extracted with ethyl acetate. The organic layer was washed with brine, dried (MgSO₄), filtrated and concentrated. The obtained residue was subjected to silica gel column chromatography, and eluted with ethyl acetate to give colorless crystals. Recrystallization from ethyl acetate-hexane gave 3-[2-[(3,5-dichloropyridin-2-yl)oxy]-4-(2-methoxyethoxy)phenyl]propyl (aminosulfonyl)carbamate (210 mg, yield: 58%) as colorless crystals. melting point 153.2-153.8° C.

### Example 427

To a solution of 3-[2-[(3,5-dichloropyridin-2-yl)oxy]-4-(2-methoxyethoxy)phenyl]propan-1-ol (404 mg) in toluene (10 ml) was added chlorosulfonylisocyanate (161 mg) under ice-cooling, and the mixture was stirred for 30 min. Pyridine (258 mg) was added to the reaction mixture and the mixture was stirred for 1 hr. 2-(2-Aminoethyl)-pyridine (796 mg) was added, and the mixture was stirred at room temperature for 4 hr. Water was added to the reaction mixture and the mixture was extracted with ethyl acetate. The organic layer was washed with brine, dried (MgSO₄), filtrated and concentrated. The obtained residue was subjected to silica gel column chromatography, and eluted with ethyl acetate-hexane (1:1′-7:3, v/v) to give 3-[2-[(3,5-dichloropyridin-2-yl)oxy]-4-(2-methoxyethoxy)phenyl]propyl {[(2-pyridin-2-ylethyl)amino]sulfonyl}carbamate (328 g, yield: 50%) as a colorless oil.

¹H-NMR (300 MHz, CDCl₃) δ:1.82-1.95 (2H, m), 2.54 (2H, t, J=7.4 Hz), 3.06 (2H, t, J=6.3 Hz), 3.43 (3H, s), 3.48-3.54 (2H, m), 3.72 (2H, dd, J=5.5, 4.0 Hz), 4.03-4.14 (4H, m), 6.60-6.66 (1H, m), 6.76 (1H, dd, J=8.5, 2.6 Hz), 7.10-7.19 (3H, m), 7.57-7.65 (1H, m), 7.76-7.80 (1H, m), 7.95 (1H, d, J=2.4 Hz), 8.47-8.52 (1H, m).

### Example 428

To a solution of 3-[2-[(3,5-dichloropyridin-2-yl)oxy]-4-(2-methoxyethoxy)phenyl]propan-1-ol (406 mg) in toluene (10 ml) was added chlorosulfonylisocyanate (162 mg) under ice-cooling, and the mixture was stirred for 30 min. Pyridine (259 mg) was added to the reaction mixture and the mixture was stirred for 1 hr. 3-Methoxypropylamine (584 mg) was added, and the mixture was stirred at room temperature for 4 hr. Water was added to the reaction mixture and the mixture was extracted with ethyl acetate. The organic layer was washed with brine, dried (MgSO₄), filtrated and concentrated. The obtained residue was subjected to silica gel column chromatography, and eluted with ethyl acetate-hexane (2:3-3:2, v/v) to give colorless crystals. Recrystallization from ethyl acetate-hexane gave 3-[2-[(3,5-dichloropyridin-2-yl)oxy]-4-(2-methoxyethoxy)phenyl]propyl {[(3-methoxypropyl)amino]sulfonyl}carbamate (435 mg, yield: 70%) as colorless crystals. melting point 87.9-89.0° C.

### Example 429

To a solution of 3-[2-[(3,5-dichloropyridin-2-yl)oxy]-4-(2-methoxyethoxy)phenyl]propan-1-ol (400 mg) in toluene (10 ml) was added chlorosulfonylisocyanate (160 mg) under ice-cooling, and the mixture was stirred for 30 min. Pyridine (255 mg) was added to the reaction mixture and the mixture was stirred for 1 hr. 2-Aminoethyl isopropyl ether (665 mg) was added, and the mixture was stirred at room temperature for 4 hr. Water was added to the reaction mixture and the mixture was extracted with ethyl acetate. The organic layer was washed with brine, dried (MgSO₄), filtrated and concentrated. The obtained residue was subjected to silica gel column chromatography, and eluted with ethyl acetate-hexane (1:3-1:1, v/v) to give colorless crystals. Recrystallization from ethyl acetate-hexane gave 3-[2-[(3,5-dichloropyridin-2-yl)oxy]-4-(2-methoxyethoxy)phenyl]propyl {[(2-isopropoxyethyl)amino]sulfonyl}carbamate (256 mg, yield: 41%) as colorless crystals. melting point 87.5-88.4° C.

### Example 430

To a solution of 3-{2-{[3-chloro-5-(trifluoromethyl)pyridin-2-yl]oxy}-4-[2-ethoxy-1-(ethoxymethyl)ethoxy]phenyl}propan-1-ol (500 mg) in toluene (13 ml) was added chlorosulfonylisocyanate (155 mg) under ice-cooling, and the mixture was stirred for 30 min. Pyridine (248 mg) was added to the reaction mixture and the mixture was stirred for 1 hr. 2-Aminoethyl isopropyl ether (648 mg) was added, and the mixture was stirred at room temperature for 4 hr. Water was added to the reaction mixture and the mixture was extracted with ethyl acetate. The organic layer was washed with brine, dried (MgSO₄), filtrated and concentrated. The obtained residue was subjected to HPLC (acetonitrile:water, containing 0.01% TFA, 5:95-100:0, v/v) to give colorless crystals. Recrystallization from ethyl acetate-hexane gave 3-{2-{[3-chloro-5-(trifluoromethyl)pyridin-2-yl]oxy}-4-[2-ethoxy-1-(ethoxymethyl)ethoxy]phenyl}propyl {[(2-isopropoxyethyl)amino]sulfonyl}carbamate (145 mg, yield: 20%) as colorless crystals. melting point 68.0-69.2° C.

### Example 431

To a solution of (2E)-3-[2-{[3-chloro-5-(trifluoromethyl)pyridin-2-yl]oxy}-4-(2-methoxyethoxy)phenyl]prop-2-en-1-ol (0.60 g) in acetonitrile (5 ml) was added chlorosulfonylisocyanate (0.14 ml) under ice-cooling, and the mixture was stirred for 30 min. Pyridine (0.44 ml) was added to the reaction mixture, and the mixture was stirred for 1 hr. (2-Aminoethyl)isopropyl ether (0.77 g) was added, and the mixture was stirred while warming to room temperature rover 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-8:2, v/v), concentrated, and crystallized from ethyl acetate-hexane to give (2E)-3-[2-{[3-chloro-5-(trifluoromethyl)pyridin-2-yl]oxy}-4-(2-methoxyethoxy)phenyl]prop-2-en-1-yl {[(2-isopropoxyethyl)amino]sulfonyl}carbamate (0.35 g, yield: 38%) as colorless crystals. melting point 127-128° C.

### Example 432

To a solution of (2E)-3-[2-{[3-chloro-5-(trifluoromethyl)pyridin-2-yl]oxy}-4-(2-methoxyethoxy)phenyl]prop-2-en-1-ol (0.60 g) in acetonitrile (5 ml) was added chlorosulfonylisocyanate (0.14 ml) under ice-cooling, and the mixture was stirred for 30 min. Pyridine (0.44 ml) was added to the reaction mixture, and the mixture was stirred for 1 hr. 3-Methoxypropylamine (0.66 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-8:2, v/v), concentrated, and crystallized from ethyl acetate-hexane to give (2E)-3-[2-{[3-chloro-5-(trifluoromethyl)pyridin-2-yl]oxy}-4-(2-methoxyethoxy)phenyl]prop-2-en-1-yl {[(3-methoxypropyl)amino]sulfonyl}carbamate (0.42 g, yield: 47%) as colorless crystals. melting point 98-100° C.

### Example 433

To a solution of (2E)-3-[2-{[3-chloro-5-(trifluoromethyl)pyridin-2-yl]oxy}-4-(2-methoxyethoxy)phenyl]prop-2-en-1-ol (0.60 g) in acetonitrile (5 ml) was added chlorosulfonylisocyanate (0.14 ml) under ice-cooling, and the mixture was stirred for 30 min. Pyridine (0.44 ml) was added to the reaction mixture and the mixture was stirred for 1 hr. 1-Pentylamine (0.65 g) was added and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-1:1, v/v), concentrated, and crystallized from ethyl acetate-hexane to give (2E)-3-[2-{[3-chloro-5-(trifluoromethyl)pyridin-2-yl]oxy}-4-(2-methoxyethoxy)phenyl]prop-2-en-1-yl [(pentylamino)sulfonyl]carbamate (0.23 g, yield: 26%) as colorless crystals.

¹H-NMR (300 MHz, CDCl₃) δ:0.81-0.94 (3H, m), 1.19-1.39 (4H, m), 1.45-1.73 (2H, m), 2.97-3.12 (2H, m), 3.44 (3H, s), 3.74 (2H, dd, J=5.5, 4.0 Hz), 4.02-4.18 (2H, m), 4.72 (2H, dd, J=6.6, 1.1 Hz), 4.91-5.05 (1H, m), 6.09-6.26 (1H, m), 6.58-6.71 (2H, m), 6.83-6.94 (1H, m), 7.13 (1H, br.s.), 7.48-7.56 (1H, m), 8.01 (1H, d, J=1.7 Hz), 8.25 (1H, dd, J=2.1, 0.9 Hz).

### Example 434

To absolution of (2E)-3-[2-{[3-chloro-5-(trifluoromethyl)pyridin-2-yl]oxy}-4-(2-methoxyethoxy)phenyl]prop-2-en-1-ol (0.60 g) in acetonitrile (5 ml) was added chlorosulfonylisocyanate (0.14 ml) under ice-cooling, and the mixture was stirred for 30 min. Pyridine (0.44 ml) was added to the reaction mixture, the mixture was stirred for 1 hr, 1-(2-aminoethyl)pyrrolidine (0.85 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with methanol-ethyl acetate (0:1-8:2, v/v), concentrated and crystallized from ethyl acetate-hexane to give (2E)-3-[2-{[3-chloro-5-(trifluoromethyl)pyridin-2-yl]oxy}-4-(2-methoxyethoxy)phenyl]prop-2-en-1-yl {[(2-pyrrolidin-1-ylethyl)amino]sulfonyl}carbamate (0.40 g, yield: 43%) as colorless crystals. melting point 154-156° C.

### Example 435

To a solution of (2E)-3-[2-{[3-chloro-5-(trifluoromethyl)pyridin-2-yl]oxy}-4-(2-methoxyethoxy)phenyl]prop-2-en-1-ol (0.60 g) in acetonitrile (5 ml) was added chlorosulfonylisocyanate (0.14 ml) under ice-cooling, and the mixture was stirred for 30 min. Pyridine (0.44 ml) was added to the reaction mixture, the mixture was stirred for 1 hr, phenethylamine (0.90 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-6:4, v/v), concentrated, and crystallized from ethyl acetate-hexane to give (2E)-3-[2-{[3-chloro-5-(trifluoromethyl)pyridin-2-yl]oxy}-4-(2-methoxyethoxy)phenyl]prop-2-en-1-yl {[(2-phenylethyl)amino]sulfonyl}carbamate (0.49 g, yield: 53%) as colorless crystals. melting point 113-114° C.

### Example 436

To a solution of (2E)-3-[2-{[3-chloro-5-(trifluoromethyl)pyridin-2-yl]oxy}-4-(2-methoxyethoxy)phenyl]prop-2-en-1-ol (0.60 g) in acetonitrile (5 ml) was added chlorosulfonylisocyanate (0.14 ml) under ice-cooling, and the mixture was stirred for 30 min. Pyridine (0.44 ml) was added to the reaction mixture, the mixture was stirred for 1 hr, 2-(2-aminoethyl)pyridine (1.46 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (2:8-9:1, v/v), concentrated, and crystallized from ethyl acetate-hexane to give (2E)-3-[2-{[3-chloro-5-(trifluoromethyl)pyridin-2-yl]oxy}-4-(2-methoxyethoxy)phenyl]prop-2-en-1-yl {[(2-pyridin-2-ylethyl)amino]sulfonyl}carbamate (0.43 g, yield: 46%) as colorless crystals.

¹H-NMR (300 MHz, CDCl₃) δ:3.05 (2H, J=6.2 Hz), 3.44 (3H, s), 3.53 (2H, t, J=6.2 Hz), 3.74 (2H, dd, J=5.4, 3.9 Hz), 4.05-4.15 (2H, m), 4.67 (2H, dd, J=6.4, 0.9 Hz), 6.06-6.23 (1H, m), 6.57-6.70 (2H, m), 6.86 (1H, dd, J=8.7, 2.4 Hz), 7.08-7.18 (2H, m), 7.49 (1H, d, J=8.9 Hz), 7.54-7.66 (1H, m), 8.00 (1H, d, J=2.1 Hz), 8.23 (1H, d, J=0.9 Hz), 8.44-8.54 (1H, m).

### Example 437

To a solution of (2E)-3-[2-{[3-chloro-5-(trifluoromethyl)pyridin-2-yl]oxy}-4-(2-methoxyethoxy)phenyl]prop-2-en-1-ol (0.50 g) in acetonitrile (5 ml) was added chlorosulfonylisocyanate (0.11 ml) under ice-cooling, and the mixture was stirred for 30 min. Pyridine (0.37 ml) was added to the reaction mixture, the mixture was stirred for 1 hr, pyrrolidine (0.44 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-6:4, v/v), concentrated, and crystallized from ethyl acetate-hexane to give (2E)-3-[2-{[3-chloro-5-(trifluoromethyl)pyridin-2-yl]oxy}-4-(2-methoxyethoxy)phenyl]prop-2-en-1-yl (pyrrolidin-1-ylsulfonyl)carbamate (0.53 g, yield: 73%) as colorless crystals. melting point 123-124° C.

### Example 438

To a solution of 3-(4-(2-{[tert-butyl(diphenyl)silyl]oxy}ethoxy)-2-{[3-chloro-5-(trifluoromethyl)pyridin-2-yl]oxy}phenyl)propan-1-ol (1.00 g) in acetonitrile (5 ml) was added chlorosulfonylisocyanate (0.15 ml) under ice-cooling, and the mixture was stirred for 30 min. Pyridine (0.47 ml) was added to the reaction mixture, and the mixture was stirred for 1 hr. (2-Aminoethyl)isopropyl ether (0.82 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-1:1, v/v) to give 3-(4-(2-{[tert-butyl(diphenyl)silyl]oxy}ethoxy)-2-{[3-chloro-5-(trifluoromethyl)pyridin-2-yl]oxy}phenyl)propyl {[(2-isopropoxyethyl)amino]sulfonyl}carbamate (0.88 g, yield: 66%) as a colorless oil.

¹H-NMR (300 MHz, CDCl₃) δ:1.04 (9H, s), 1.13 (6H, d, J=6.2 Hz), 1.85-2.01 (2H, m), 2.54 (2H, t, J=7.4 Hz), 3.16-3.29 (2H, m), 3.44-3.63 (3H, m), 3.89-4.20 (6H, m), 5.39 (1H, t, J=5.7 Hz), 6.63 (1H, d, J=2.4 Hz), 6.77 (1H, dd, J 8.5, 2.6 Hz), 7.17 (1H, d, J=8.5 Hz), 7.31-7.48 (7H, m), 7.62-7.74 (7H, m), 8.01 (1H, d, J=1.7 Hz), 8.26 (1H, dd, J=2.2, 1.0 Hz).

### Example 439

To a solution of 3-(4-(2-{[tert-butyl(diphenyl)silyl]oxy}ethoxy)-2-{[3-chloro-5-(trifluoromethyl)pyridin-2-yl]oxy}phenyl)propyl {[(2-isopropoxyethyl)amino]sulfonyl}carbamate (0.88 g) in tetrahydrofuran (5 ml) were added a tetra-n-butylammonium fluoride 1.0M-tettahydrofuran solution (5.3 ml), and the mixture was stirred at room temperature for 6 hr. Water was added to the reaction mixture, and the mixture was extracted with ethyl acetate. The ethyl acetate layer was washed with saturated brine, dried (MgSO₄), and concentrated. The residue was subjected to HPLC (acetonitrile:water, containing 0.01% TFA, 5:95-100:0, v/v) to give 3-[2-{[3-chloro-5-(trifluoromethyl)pyridin-2-yl]oxy}-4-(2-hydroxyethoxy)phenyl]propyl {[(2-isopropoxyethyl)amino]sulfonyl}carbamate (0.32 g, yield: 51%) as a colorless amorphous solid.

¹H-NMR (300 MHz, CDCl₃) δ:1.14 (6H, d, J=6.2 Hz), 1.88-2.00 (2H, m), 2.01-2.13 (1H, m), 2.58 (2H, t, J=7.3 Hz), 3.23 (2H, q, J=5.5 Hz), 3.47-3.65 (3H, m), 3.95 (2H, d, J=4.1 Hz), 4.03-4.19 (4H, m), 5.39 (1H, t, J=5.8 Hz), 6.69 (1H, d, J=2.4 Hz), 6.83 (1H, dd, J=8.5, 2.4 Hz), 7.20 (1H, d, J=8.5 Hz), 7.54 (1H, br.s.), 8.02 (1H, d, J=2.3 Hz), 8.25-8.31 (1H, m)

### Example 440

To a solution of 3-(2-{[3-chloro-5-(trifluoromethyl)pyridin-2-yl]oxy}-4-isopropoxyphenyl)propan-1-ol (0.4.0 g) in acetonitrile (5 ml) was added chlorosulfonylisocyanate (0.09 ml) under ice-cooling, and the mixture was stirred for 30 min. Pyridine (0.30 ml) was added to the reaction mixture, and the mixture was stirred for 1 hr. 3-Methoxypropylamine (0.48 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-4:6, v/v), concentrated, and crystallized from ethyl acetate-hexane to give 3-(2-{[3-chloro-5-(trifluoromethyl)pyridin-2-yl]oxy}-4-isopropoxyphenyl)propyl {[(3-methoxypropyl)amino]sulfonyl}carbamate (0.44 g, yield: 74%) as colorless crystals.

¹H-NMR (300 MHz, CDCl₃) δ:1.33 (6H, d, J=6.0 Hz), 1.75-2.02 (4H, m), 2.55 (2H, t, J=7.5 Hz), 3.12-3.23 (2H, m), 3.32 (3H, s), 3.44-3.51 (2H, t, J=5.6 Hz), 4.15 (2H, t, J=6.4 Hz), 4.43-4.54 (1H, m), 5.57-5.66 (1H, m), 6.62 (1H, d, J=2.4 Hz), 6.77 (1H, dd, J=8.5, 2.6 Hz), 7.17 (1H, d, J=8.5 Hz), 8.01 (1H, d, J=2.1 Hz), 8.28 (1H, d, J=1.3 Hz).

### Example 441

To a solution of 3-(2-{[3-chloro-5-(trifluoromethyl)pyridin-2-yl]oxy}-4-isopropoxyphenyl)propan-1-ol (0.40 g) in acetonitrile (5 ml) was added chlorbsulfonylisocyanate (0.09 ml) under ice-cooling, and the mixture was stirred for 30 min. Pyridine (0.30 ml) was added to the reaction mixture, and the mixture was stirred for 1 hr. (2-Aminoethyl)isopropyl ether (0.56 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-3:7, v/v), concentrated, and crystallized from ethyl acetate-hexane to give 3-(2-{[3-chloro-5-(trifluoromethyl)pyridin-2-yl]oxy}-4-isopropoxyphenyl)propyl {[(2-isopropoxyethyl)amino]sulfonyl}carbamate (0.32 g, yield: 51%) as colorless crystals.

¹H-NMR (300 MHz, CDCl₃) δ:1.14 (6H, d, J=6.2 Hz), 1.32 (6H, d, J=6.0 Hz), 1.86-2.02 (2H, m), 2.54 (2H, t, J=7.3 Hz), 3.23 (2H, q, J=5.5 Hz), 3.43-3.65 (3H, m), 4.15 (2H, t, J=6.4 Hz), 4.39°-4.57 (1H, m), 5.37 (1H, t, J=5.7 Hz), 6.62 (1H, d, J=2.6 Hz), 6.77 (1H, dd, J=8.4, 2.5 Hz), 7.17 (1H, d, J=8.5 Hz), 7.37 (1H, br.s.), 8.00 (1H, d, J=2.3 Hz), 8.28 (1H, d, J=1.1 Hz).

### Example 442

To a solution of 3-(2-{[3-chloro-5-(trifluoromethyl)pyridin-2-yl]oxy}-4-isopropoxyphenyl)propan-1-ol (0.40 g) in acetonitrile (5 ml) was added chlorosulfonylisocyanate (0.09 ml) under ice-cooling, and the mixture was stirred for 30 min. Pyridine (0.30 ml) was added to the reaction mixture, and the mixture was stirred for 1 hr. 2-Phenoxyethylamine (0.74 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-3:7, v/v), concentrated, and crystallized from ethyl acetate-hexane to give 3-(2-{[3-chloro-5-(trifluoromethyl)pyridin-2-yl]oxy}-4-isopropoxyphenyl)propyl {[(2-phenoxyethyl)amino]sulfonyl}carbamate (0.30 g, yield: 46%) as colorless crystals.

¹H-NMR (300 MHz, CDCl₃) δ:1.32 (6H, d, J=6.0 Hz), 1.79-1.98 (2H, m), 2.53 (2H, t, J=7.3. Hz), 3.41-3.56 (2H, m), 4.01-4.15 (4H, m), 4.39-4.57 (1H, m), 5.45-5.56 (1H, m), 6.62 (1H, d, J=2.6 Hz), 6.76 (1H, dd, J=8.5, 2.4 Hz), 6.82-6.90 (2H, m), 6.92-7.01 (1H, m), 7.14 (1H, d, J=8.5 Hz), 7.23-7.31 (2H, m), 7.40 (1H, br.s.), 8.00 (1H, d, J=1.9 Hz), 8.28 (1H, dd, J=2.1, 0.9 Hz).

### Example 443

To a solution of 3-(2-{[3-chloro-5-(trifluoromethyl)pyridin-2-yl]oxy}-4-isopropoxyphenyl)propan-1-ol (0.40 g) in acetonitrile (5 ml) was added chlorosulfonylisocyanate (0.09 ml) under ice-cooling, and the mixture was stirred for 30 min. Pyridine (0.30 ml) was added to the reaction mixture, and the mixture was stirred for 1 hr. N-(2-Methoxyethyl)methylamine (0.48 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-8:2, v/v), concentrated, and crystallized from ethyl acetate-hexane to give 3-(2-{[3-chloro-5-(trifluoromethyl)pyridin-2-yl]oxy}-4-isopropoxyphenyl)propyl {[(2-methoxyethyl)(methyl)amino]sulfonyl}carbamate (0.42 g, yield: 70%) as a colorless amorphous solid.

¹H-NMR (300 MHz, CDCl₃) δ:1.32 (6H, d, J=6.0 Hz), 1.86-2.01 (2H, m), 2.54 (2H, t, J=7.4 Hz), 3.00 (3H, s), 3.36 (3H, s), 3.44-3.61 (4H, m), 4.12 (2H, t, J=6.5 Hz), 4.40-4.57 (1H, m), 6.62 (1H, d, J=2.4 Hz), 6.77 (1H, dd, J 8.5, 2.4 Hz), 7.17 (1H, d, J=8.5 Hz), 7.48 (1H, br.s.), 8.00 (1H, d, J=1.9 Hz), 8.28 (1H, dd, J=2.1, 0.9 Hz).

### Example 444

To a solution of 3-(2-{[3-chloro-5-(trifluoromethyl)pyridin-2-yl]oxy}-4-isopropoxyphenyl)propan-1-ol (0.40 g) in acetonitrile (5 ml) was added chlorosulfonylisocyanate (0.09 ml) under ice-cooling, and the mixture. was stirred for 30 min. Pyridine (0.30 ml) was added to the reaction mixture, and the mixture was stirred for 1 hr. 2-(2-Aminoethyl)pyridine (0.66 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (0:1-8:2, v/v) to give 3-(2-{[3-chloro-5-(trifluoromethyl)pyridin-2-yl]oxy}-47 isopropoxyphehyl)propyl {[(2-pyridin-2-ylethyl)amino]sulfonyl}carbamate (0.33 g, yield: 52%) as a colorless amorphous solid.

¹H-NMR (300 MHz, CDCl₃) δ:1.31 (6H, d, J=6.0 Hz), 1.74-1.94 (2H, m), 2.46 (2H, t, J=7.4 Hz), 3.03 (2H, t, J=6.6 Hz), 3.47 (2H, t, J=6.5 Hz), 4.04 (2H, t, J=6.3 Hz), 4.36-4.57 (1H, m), 6.60 (1H, d, J=2.4 Hz), 6.72 (1H, dd, J 8.5, 2.4 Hz), 7.02-7.20 (3H, m), 7.58 (1H, td, J=7.7, 1.6 Hz), 7.96 (1H, d, J=2.1 Hz), 8.24 (1H, d, J=1.1 Hz), 8.47 (1H, d, J=4.3 Hz).

### Example 445

To a solution of 2-[2-(benzyloxy)-4-(2-methoxyethoxy)phenyl]ethanol (0.30 g) in acetonitrile (5 ml) was added chlorosulfonylisocyanate (0.07 ml) under ice-cooling, and the mixture was stirred for 30 min. Pyridine (0.23 ml) was added to the reaction mixture, and the mixture was stirred for 1 hr. (2-Aminoethyl)isopropyl ether (0.42 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-8:2, v/v), concentrated, and crystallized from ethyl acetate-hexane to: give 2-[2-{[3-chloro-5-(trifluoromethyl)pyridin-2-yl]oxy}-4-(2-methoxyethoxy)phenyl]ethyl {[(2-isopropoxyethyl)amino]sulfonyl}carbamate (0.26 g, yield: 56%) as colorless crystals.

¹H-NMR (300 MHz, CDCl₃) δ:1.13 (6H, d, J=6.2 Hz), 2.86 (2H, t, J=6.8 Hz), 3.16 (2H, q, J=5.7 Hz), 3.43 (3H, s), 3.46-3.64 (3H, m), 3.73 (2H, dd, J=5.5, 4.0 Hz), 4.03-4.14 (2H, m), 4.34 (2H, t, J=6.9 Hz), 5.38 (1H, t, J=5.8 Hz), 6.66 (1H, d, J=2.4 Hz), 6.83 (1H, dd, J=8.5, 2.6 Hz), 7.23 (1H, d, J=8.7 Hz), 7.39 (1H, br. s.), 8.02 (1H, d, J=2.1 Hz), 8.25-8.33 (1H, m).

### Example 446

To a solution of 2-[2-(benzyloxy)-4-(2-methoxyethoxy)phenyl]ethanol (0.30 g) in acetonitrile (5 ml) was added chlorosulfonylisocyanate (0.07 ml) under ice-cooling, and the mixture was stirred for 30 min. Pyridine (0.23 ml) was added to the reaction mixture, and the mixture was stirred for 1 hr. (2-Aminopropyl)isopropyl ether (0.48 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-8:2, v/v), concentrated, and crystallized from ethyl acetate-hexane to give 2-[2-{[3-chloro-5-(trifluoromethyl)pyridin-2-yl]oxy}-4-(2-methoxyethoxy)phenyl]ethyl {[(3-isopropoxypropyl)amino]sulfonyl}carbamate (0.31 g, yield: 66%) as colorless crystals.

¹H-NMR (300 MHz, CDCl₃) δ:1.13 (6H, d, J=6.2 Hz), 2.86 (2H, t, J=6.8 Hz), 3.16 (2H, q, J=5.7 Hz), 3.43 (3H, s), 3.46-3.64 (3H, m), 3.73 (2H, dd, J=5.5, 4.0 Hz), 4.03-4.14 (2H, m), 4.34 (2H, t, J=6.9 Hz), 5.38 (1H, t, J=5.8 Hz), 6.66 (1H, d, J=2.4 Hz), 6.83 (1H, dd, J=8.5, 2.6 Hz), 7.23 (1H, d, J=8.7 Hz), 7.39 (1H, br.s.), 8.02 (1H, d, J=2.1 Hz), 8.25-8.33 (1H, m).

### Example 447

To a solution of 2-[2-(benzyloxy)-4-(2-methoxyethoxy)phenyl]ethanol (0.30 g) in acetonitrile (5 ml) was added chlorosulfonylisocyanate (0.07 ml) under ice-cooling, and the mixture was stirred for 30 min. Pyridine (0.23 ml) was added to the reaction mixture, and the mixture was stirred for 1 hr. 3-Methoxypropylamine (0.36 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-9:1, v/v), concentrated, and crystallized from ethyl acetate-hexane to give 2-[2-{[3-chloro-5-(trifluoromethyl)pyridin-2-yl]oxy}-4-(2-methoxyethoxy)phenyl]ethyl {[(3-methoxypropyl)amino]sulfonyl}carbamate (0.27 g, yield: 60%) as colorless crystals.

¹H-NMR (300 MHz, CDCl₃) δ:1.70-1.83 (2H, m), 2.87 (2H, t, J=6.8 Hz), 3.09 (2H, q, J=6.2 Hz), 3.31 (3H, s), 3.38-3.49 (5H, m), 3.73 (2H, dd, J=5.6, 3.9 Hz), 4.08 (2H, dd, J=5.5, 4.0 Hz), 4.35 (2H, t, J=6.9 Hz), 5.52-5.64 (1H, m), 6.65 (1H, d, J=2.4 Hz), 6.84 (1H, dd, J=8.5, 2.6 Hz), 7.20-7.25 (1, H, m), 8.02 (1H, d, J=2.1 Hz), 8.30 (1H, d, J=1.1 Hz).

### Example 448

To a solution of 2-[2-{[3-chloro-5-(trifluoromethyl)pyridin-2-yl]oxy}-4-(methoxymethoxy)phenyl]ethanol (1.32 g) in acetonitrile (30 ml) was added chlorosulfonylisocyanate (0.54 g) under ice-cooling, and the mixture was stirred for 30 min. Pyridine (1.62 ml) was added to the reaction mixture, and the mixture was stirred for 1 hr. (2-Aminoethyl)isopropyl ether (1.98 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-1:1, v/v), concentrated, and crystallized from ethyl acetate-hexane to give 2-[2-{[3-chloro-5-(trifluoromethyl)pyridin-2-yl]oxy}-4-(methoxymethoxy)phenyl]ethyl {[(2-isopropoxyethyl)amino]sulfonyl}carbamate (0.29 g, yield: 14%) as colorless crystals.

¹H-NMR (300 MHz; CDCl₃) δ:1.13 (6H, d, J=6.2 Hz), 2.86 (2H, t, J=6.8 Hz), 3.18 (2H, q, J=5.7 Hz), 3.42-3.63 (6H, m), 4.35 (2H, t, J=6.9 Hz), 5.15 (2H, s), 5.40 (1H, t, J=6.0 Hz), 6.80 (1H, d, J=2.4 Hz), 6.95 (1H, dd, J=8.5, 2.4 Hz), 7.24 (1H, d, J=8.5 Hz), 7.37 (1H, s), 8.01 (1H, d, J=1.9 Hz), 8.29 (1H, dd, J=2.2, 1.0 Hz).

### Example 449

To a solution of (2E)-3-{2-{[3-chloro-5-(trifluoromethyl)pyridin-2-yl]oxy}-4-[2-(cyclopropyloxy)ethoxy]phenyl}-N-(pentylsulfonyl)acrylamide (463 mg) in tetrahydrofuran (4 ml) and methanol (4 ml) was added platinum (IV) oxide (18 mg), and the mixture was stirred under a hydrogen atmosphere at room temperature for 2 hr. The reaction mixture was filtrated and the filtrate was concentrated to give a white solid. Recrystallization from ethyl acetate-hexane gave 3-{2-{[3-chloro-5-(trifluoromethyl)pyridin-2-yl]oxy}-4-[2-(cyclopropyloxy)ethoxy]phenyl}-N-(pentylsulfonyl)propanamide (395.5 mg, yield: 85%) as white crystals.

¹H-NMR (300 MHz, CDCl₃) δ:0.43-0.54 (2H, m), 0.55-0.63 (2H, m), 0.90 (3H, t, J=7.0 Hz), 1.14-1.43 (4H, m), 1.59-1.72 (2H, m), 2.55 (2H, t, J=7.2 Hz), 2.95 (2H, t, J=7.2 Hz), 3.10-3.21 (2H, m), 3.36 (1H, tt, J=6.1, 3.0 Hz), 3.71-3.83 (2H, m), 3.91-4.08 (2H, m), 6.51 (1H, d, J=2.7 Hz), 6.77 (1H, dd, J=8.3, 2.7 Hz), 7.18 (1H, d, J=8.7 Hz), 8.08 (1H, d, J=2.3 Hz), 8.37 (1H, dd, J=2.1, 0.9 Hz)

### Example 450

To a solution of 3-[2-{[3-chloro-5-(trifluoromethyl)pyridin-2-yl]oxy}-4-(methoxymethoxy)phenyl]propan-1-ol (5.2 g) in acetonitrile (100 ml) was added chlorosulfonylisocyanate (2.1 g) under ice-cooling, and the mixture was stirred for 30 min. Pyridine (4.1 ml) was added to the reaction mixture, and the mixture was stirred for 1 hr. (2-Aminoethyl)isopropyl ether (6.8 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-6:4, v/v), concentrated, and crystallized from ethyl acetate-hexane to give, 3-[2-{[3-chloro-5-(trifluoromethyl)pyridin-2-yl]oxy}-4-. (methoxymethoxy)phenyl]propyl {[(2-isopropoxyethyl)amino]sulfonyl}carbamate (5.2 g, yield: 65%) as colorless crystals.

¹H-NMR (300 MHz, CDCl₃) δ:1.14 (6H, d, J=6.0 Hz), 1.88-2.01 (2H, m), 2.56 (2H, t, J=7.4 Hz), 3.23 (2H, q, J=5.7 Hz), 3.48 (3H, s), 3.49-3.62 (3H, m), 4.15 (2H, t, J=6.4 Hz), 5.15 (2H, s), 5.39 (1H, t, J=5.9 Hz), 6.82 (1H, d, J=2.7 Hz), 6.94 (1H, dd, J=8.3, 2.7 Hz), 7.20 (1H, d, J=8.3 Hz), 7.42 (1H, br. s.), 8.01 (1H, d, J=2.3 Hz), 8.27 (1H, s).

### Example 451

A mixture of 1-ethyl-3-(3-dimethylaminopropyl)carbodiimide hydrochloride (1.21 g), 4-dimethylaminopyridine (892 mg), and acetonitrile (10 ml) was stirred at room temperature for 10 min. To the mixture were added (2E)-3-[2-{[3-chloro-5-(trifluoromethyl)pyridin-2-yl]oxy}-4-(3-cyanopropoxy)phenyl]acrylic acid (2.08 g) and pentane-1-sulfonamide (749 mg), and the mixture was stirred at room temperature for 15 hr. A 1M hydrochloric acid solution was added to the reaction mixture, and the mixture was extracted with ethyl acetate. The ethyl acetate layer was washed with saturated brine, dried (MgSO₄), and concentrated. The obtained solid was recrystallized from ethyl acetate-hexane to give (2E)-3-[2-{[3-chloro-5-(trifluoromethyl)pyridin-2-yl]oxy}-4-(3-cyanopropoxy)pheny]-N-(pentylsulfonyl)acrylamide (708 mg, yield: 26%) as colorless crystals. melting point 165-166° C.

### Example 452

A mixture of 1-ethyl-3-(3-dimethylaminopropyl)carbodiimide hydrochloride (150 mg), 4-dimethylaminopyridine (110 mg), and acetonitrile (3 ml) was stirred at room temperature for 10 min. To the mixture were added (2E)-3-[2-{[3-chloro-5-(trifluoromethyl)pyridin-2-yl]oxy}-4-(2-oxopropoxy)phenyl]acrylic acid (250 mg) and pentane-1-sulfonamide (93 mg), and the mixture was stirred at room temperature for 15 hr. A 1M hydrochloric acid solution was added to the reaction mixture, and the mixture was extracted with ethyl acetate. The ethyl acetate layer was washed with saturated brine, dried (MgSO₄), and concentrated. The obtained solid was recrystallized from ethyl acetate-hexane to give, (2E)-3-[2-{[3-chloro-5-(trifluoromethyl)pyridin-2-yl]oxy}-4-(2-oxopropoxy)phenyl]-N-(pentylsulfonyl)acrylamide (70 mg, yield: 30%) as colorless crystals. melting point 187-188° C.

### Example 453

A mixture of 1-ethyl-3-(3-dimethylaminopropyl)carbodiimide hydrochloride (940 mg), 4-dimethylaminopyridine (691 mg), and acetonitrile (5 ml) was stirred at room temperature for 10 min. To the mixture were added (2E)-3-{2-{[3-chloro-5-(trifluoromethyl)pyridin-2-yl]oxy}-4-[2-(2-methyl-1,3-dioxolan-2-yl)ethoxy]phenyl}acrylic acid (1.79 g) and pentane-1-sulfonamide (580 mg), and the mixture was stirred at room temperature for 15 hr. A 1M hydrochloric acid solution was added to the reaction mixture, and the mixture was extracted with ethyl acetate. The ethyl acetate layer was washed with saturated brine, dried (MgSO₄), and concentrated. The obtained solid was recrystallized from ethyl acetate-hexane to give (2E)-3-{2-{[3-chloro-5-(trifluoromethyl)pyridin-2-yl]oxy}-4-[2-(2-methyl-1,3-dioxolan-2-yl)ethoxy]phenyl}-N-(pentylsulfonyl)acrylamide (1.19 g, yield: 52%) as colorless crystals. melting point 90-92° C.

### Example 454

A mixture of (2E)-3-{2-{[3-chloro-5-(trifluoromethyl)pyridin-2-yl]oxy}-4-[2-(2-methyl-1,3-dioxolan-2-yl)ethoxy]phenyl}-N-(pentylsulfonyl)acrylamide (750 mg), 1M hydrochloric acid solution (5 ml), and tetrahydrofuran (15 ml) was stirred at 60° C. for 1 hr. After cooling to room temperature, the mixture was concentrated. Water was added to the obtained residue, and the mixture was extracted with ethyl acetate. The ethyl acetate layer was washed with saturated brine, dried (MgSO₄), and concentrated. The obtained solid was recrystallized from ethyl acetate to give (2E)-3-[2-{[3-chloro-5-(trifluoromethyl)pyridin-2-yl]oxy}-4-(3-oxobutoxy)phenyl]-N-(pentylsulfonyl)acrylamide (418 mg, yield: 60%) as colorless crystals. melting point 180-181° C.

### Example 455

A mixture of 1-ethyl-3-(3-dimethylaminopropyl)carbodiimide hydrochloride (247 mg), 4-dimethylaminopyridine (182 mg), and acetonitrile (5 ml) was stirred at room temperature for 10 min. To the mixture were added (2E)-3-(4-[2-(acetyloxy)-3-isopropoxypropoxy]-2-{[3-chloro-5-(trifluoromethyl)pyridin-2-yl]oxy}phenyl)acrylic acid (513 mg) and pentane-1-sulfonamide (152 mg), and the mixture was stirred at room temperature for 2.5 hr. A 1M hydrochloric acid solution was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:5-1:2, v/v). The obtained solid was recrystallized from diethyl ether-hexane to give 2-(3-{[3-chloro-5-(trifluoromethyl)pyridin-2-yl]oxy}-4-{(1E)-3-oxo-3-[(pentylsulfonyl)amino]prop-1-en-1-yl}phenoxy)-1-(isopropoxymethyl)ethyl acetate (487 mg, yield: 76%) as colorless crystals. melting point 101-103° C.

### Example 456

To a mixture of 2-(3-{[3-chloro-5-(trifluoromethyl)pyridin-2-yl]oxy}-4-{(1E)-3-oxo-3-[(pentylsulfonyl)amino]prop-1-en-1-yl}phenoxy)-1-(isopropoxymethyl)ethyl acetate (380 mg), tetrahydrofuran (6 ml), and ethanol (4 ml) was added a 1M sodium hydroxide solution (4 ml), and the mixture was stirred at room temperature for 30 min. The reaction mixture was poured into water, and the mixture was neutralized with a 1M hydrochloric acid solution (4.1 ml) and extracted with ethyl acetate. The ethyl acetate layer was washed with saturated brine, dried (MgSO₄), and concentrated. The residue was subjected to silica gel column chromatography, and eluted with ethyl acetate-hexane (1:4-2:1, v/v). The obtained solid was recrystallized from diethyl ether-hexane to give (2E)-3-[2-{[3-chloro-5-(trifluoromethyl)pyridin-2-yl]oxy}-4-(2-hydroxy-3-isopropoxypropoxy)phenyl]-N-(pentylsulfonyl)acrylamide (185 mg, yield: 52%) as colorless crystals. melting point 86-88° C.

### Example 457

A mixture of (2E)-3-[1-(2,4-dichlorobenzyl)-5-methyl-1H-pyrrol-2-yl]acrylic acid (600 mg), 1,1′-carbonyldiimidazole (408 mg) and N,N-dimethylformamide (7 ml) was stirred at room temperature for 1 hr. To the mixture were added pentane-1-sulfonamide (336 mg) and 1,8-diazabicyclo[5.4.0]undec-7-ene (0.434 ml), and the mixture was stirred at 100° C. for 18 hr. After cooling the reaction mixture to room temperature, a 1M hydrochloric acid solution was added, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:4-99:1, v/v). The obtained solid was recrystallized from ethyl acetate-hexane to give (2E)-3-[1-(2,4-dichlorobenzyl)-5-methyl-1H-pyrrol-2-yl]-N-(pentylsulfonyl)acrylamide (545 mg, yield: 64%) as orange crystals. melting point 189-190° C.

### Example 458

A mixture of 1-ethyl-3-(3-dimethylaminopropyl)carbodiimide hydrochloride (498 mg), 4-dimethylaminopyridine (367 mg), and acetonitrile (5 ml) was stirred at room temperature for 10 min. To the mixture were added (2E)-3-[1-(2,4-dichlorobenzyl)-3,5-dimethyl-1H-pyrrol-2-yl]acrylic acid (650 mg) and pentane-1-sulfonamide (308 mg), and the mixture was stirred at room temperature for 4 hr. A 1M hydrochloric acid solution was added to the reaction mixture, and the mixture was extracted with ethyl acetate. The ethyl acetate layer was washed with saturated brine, dried (MgSO₄), and concentrated. The residue was subjected to silica gel column chromatography, and eluted with ethyl acetate-hexane (1:5-1:3, v/v). The obtained solid was recrystallized from ethyl acetate-hexane to give (2E)-3-[1-(2,4-dichlorobenzyl)-3,5-dimethyl-1H-pyrrol-2-yl]-N-(pentylsulfonyl)acrylamide (481 mg, yield: 53%) as pale-yellow crystals. melting point 170-171° C.

### Example 459

To a mixture of (2E)-3-[2-{[3-chloro-5-(trifluoromethyl)pyridin-2-yl]oxy}-4-(2-methoxyethoxy)phenyl]acrylic acid (989 mg), 1-ethyl-3-(37 dimethylaminopropyl)carbodiimide hydrochloride (682 mg), 4-dimethylaminopyridine (522 mg) and acetonitrile (4.7 ml) was added pentane-1-sulfonamide (358 mg) at room temperature. The reaction mixture was stirred at room temperature for 16 hr, 1N hydrochloric acid (6 ml), ethyl acetate (15 ml) and water (6 ml) were successively added, and the mixture was stirred at room temperature for 10 min. The organic layer was separated and the aqueous layer was extracted with ethyl acetate (8 ml). The combined organic layer was washed successively with 1N hydrochloric acid (4 ml) and saturated brine (4 ml), and dried (MgSO₄). The solvent was evaporated under reduced pressure. The residue was subjected to silica gel column chromatography, and eluted with ethyl acetate-hexane (1:1, v/v). The obtained crude crystals were dissolved in ethyl acetate, hexane was added and the mixture was stood for 1.5 hr. Hexane (1.5 ml) was further added, and the mixture was stirred for 1.5 hr. The precipitated crystals were collected by filtration with hexane to give (2E)-3-[2-{[3-chloro-5-(trifluoromethyl)pyridin-2-yl]oxy}-4-(2-methoxyethoxy)phenyl]-N-(pentylsulfonyl)acrylamide (806 mg, yield: 62%) as colorless crystals. The obtained crystals were recrystallized from ethanol to give colorless crystals. melting point 123-125° C.

¹H-NMR (300 MHz, CDCl₃) δ:0.88 (3H, t, J=7.1 Hz), 1.24-1.50 (4H, m), 1.72-1.91 (2H, m), 3.39-3.53 (5H, m), 3.75 (2H, dd, J=5.3, 3.8 Hz), 4.14 (2H, dd, J=5.4, 3.7 Hz), 6.37 (1H, d, J=15.6 Hz), 6.70 (1H, d, J=2.5 Hz), 6.90 (1H, dd, J=8.7, 2.5 Hz), 7.60 (1H, d, J=8.7 Hz), 7.80 (1H, d, J=15.6 Hz), 8.01 (1H, s), 8.03 (1H, d, J=2.3 Hz), 8.19-8.30 (1H, m)

### Example 460

To a solution of (2E)-3-[2-[(5-bromo-3-methylpyridin-2-yl)oxy]-4-(2-methoxyethoxy)phenyl]acrylic acid (697 mg) in acetonitrile (15 ml) were added 2-methyl-6-nitrobenzoic anhydride (709 mg), triethylamine (524 mg), 4-dimethylaminopyridine (234 mg) and pentane-1-sulfonamide (286 mg), and the mixture was stirred at room temperature for 40 hr. A saturated aqueous ammonium chloride solution was poured into the reaction mixture, and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:1, v/v). The obtained crude crystals were recrystallized from ethanol-hexane to give (2E)-3-[2-[(5-bromo-3-methylpyridin-2-yl)oxy]-4-(2-methoxyethoxy)phenyl]-N-(pentylsulfonyl)acrylamide (765 mg, yield: 83%) as colorless crystals. melting point 159.8-160.2° C.

### Example 461

To a solution of (2Z)-3-[2-{[3-chloro-5-(trifluoromethyl)pyridin-2-yl]oxy}-4-(3-methoxypropoxy)phenyl]-2-methoxyacrylic acid (450 mg) in acetonitrile (10 ml) were added 2-methyl-6-nitrobenzoic anhydride (404 mg), triethylamine (300 mg), 4-dimethylaminopyridine (118 mg) and pentane-1-sulfonamide (164 mg), and the mixture was stirred at room temperature for 20 hr. A saturated aqueous ammonium chloride solution was poured into the reaction mixtures and the mixture was extracted with ethyl acetate. The ethyl acetate layer was washed with water and saturated brine, dried (MgSO₄), and concentrated. The residue was subjected to silica gel column chromatography, and eluted with ethyl acetate-hexane (1:3, v/v) to give (2Z)-3-[2-{[3-chloro-5-(trifluoromethyl)pyridin-2-yl]oxy}-4-(3-methoxypropoxy)phenyl]-2-methoxy-N-(pentylsulfonyl)acrylamide (300 mg, yield: 51%) as an amorphous solid.

¹H-NMR (300 MHz, CDCl₃) δ:0.89 (3H, t, J=7.0 Hz), 1.25-1.45 (4H, m), 1.79-1.88 (2H, m), 2.01-2.09 (2H, m), 3.34 (3H, s), 3.42-3.49 (2H, m), 3.54 (2H, t, J=6.1 Hz), 3.66 (3H, s), 4.08 (2H, t, J=6.1 Hz), 6.71 (1H, d, J=2.7 Hz), 6.90 (1H, dd, J=9.1, 2.7 Hz), 7.17 (1H, s), 7.91 (1H, d, J=9.1 Hz), 8.00 (1H, d, J=1.9 Hz), 8.21 (1H, s), 8.65 (1H, s)

### Example 462

To a solution of tert-butyl[6-(5-(2-methoxyethoxy)-2-{(1E)-3-oxo-3-[(pentylsulfonyl)amino]prop-1-en-1-yl}phenoxy)-5-methylpyridin-3-yl]carbamate (327 mg) in methanol (3 ml) was added 10% hydrogen chloride methanol solution (3 ml), and the mixture was stirred at room temperature for 14 hr and further at 45° C. for 3 hr. The reaction mixture was concentrated, and the obtained crude crystals were recrystallized from methanol-diisopropyl ether to give (2E)-3-[2-[(5-amino-3-methylpyridin-2-yl)oxy]-4-(2-methoxyethoxy)phenyl]-N-(pentylsulfonyl)acrylamide dihydrochloride (207 mg, yield: 66%) as colorless crystals.

¹H-NMR (300 MHz, DMSO-d₆) δ:0.80-0.85 (3H, m), 1.14-1.29 (4H, m), 1.30-1.49 (2H, m), 2.39 (3H, s), 3.09-3.27 (4H, m), 3.30 (3H, s), 3.65-3.67 (2H, m), 4.13-4.16 (2H, m), 6.21-6.24 (1H, m), 6.96 (1H, dd, J=8.7, 2.4 Hz), 7.05 (1H, d, J=2.4 Hz), 7.39 (1H, d, J=8.7 Hz), 7.66-7.67 (2H, m), 11.93 (1H, s)

### Example 463

To a solution of (2E)-3-[2-[(5-amino-3-methylpyridin-2-yl)oxy]-4-(2-methoxyethoxy)phenyl]-N-(pentylsulfonyl)acrylamide dihydrochloride (321 mg) in pyridine (5 ml) were added acetic anhydride (445 mg) and 4-dimethylaminopyridine (145 mg), and the mixture was stirred at room temperature for 7 hr. The reaction mixture was concentrated, a saturated aqueous ammonium chloride solution was added to the obtained residue, and the mixture was extracted with ethyl acetate. The ethyl acetate layer was washed with saturated brine, dried (MgSO₄), and concentrated. The obtained residue was subjected to silica gel column chromatography, and eluted with ethyl acetate. The obtained crude crystals were recrystallized from ethanol-hexane to give (2E)-3-[2-{[5-(acetylamino)-3-methylpyridin-2-yl]oxy}-4-(2-methoxyethoxy)phenyl]-N-(pentylsulfonyl)acrylamide (150 mg, yield: 49%) as colorless crystals. melting point 187.9-188.2° C.

### Example 464

To a solution of (2E)-3-[2-[(5-amino-3-methylpyridin-2-yl)oxy]-4-(2-methoxyethoxy)phenyl]-N-(pentylsulfonyl)acrylamide dihydrochloride (356 mg) in pyridine (5 ml) were added methanesulfonyl chloride (398 mg) and 4-dimethylaminopyridine (132 mg), and the mixture was stirred at room temperature for 8 hr. The reaction mixture was concentrated, water was added to the obtained residue, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:1, v/v). The obtained crude crystals were recrystallized from ethyl acetate-hexane to give (2E)-3-[2-({5-[bis(methylsulfonyl)amino]-3-methylpyridin-2-yl}oxy)-4-(2-methoxyethoxy)phenyl]-N-(pentylsulfonyl)acrylamide (100 mg, yield: 24%) as colorless crystals.

¹H-NMR (300 MHz, CDCl₃) δ:0.88 (3H, t, J=7.1 Hz), 1.25-1.41 (4H, m), 1.75-1.88 (2H, m), 2.44 (3H, s), 3.40 (6H, s), 3.43 (3H, s), 3.43-3.45 (2H, m), 3.72-3.75 (2H, m), 4.11-4.14 (2H, m), 6.28 (1H, d, J=15.7 Hz), 6.67 (1H, d, J=2.5 Hz), 6.84 (1H, dd, J=8.7, 2.5 Hz), 7.53-7.56 (2H, m), 7.82 (1H, d, J=15.7 Hz), 7.91 (1H, d, J=2.5 Hz), 8.10 (1H, s).

### Example 465

To a solution of 3-[2-hydroxy-4-(2-methoxyethoxy)phenyl]propyl (cyclopropylmethyl)carbamate (470 mg) in N,N-dimethylformamide (6 ml) were added ethyl 5,6-dichloronicotinate (476 mg) and potassium carbonate (503 mg), and the mixture was stirred at 80° C. for 1 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (1:2, v/v). The obtained crude crystals were recrystallized from ethyl acetate-hexane to give ethyl 5-chloro-6-[2-[3-({[(cyclopropylmethyl)amino]carbonyl}oxy)propyl]-5-(2-methoxyethoxy)phenoxy]nicotinate (698 mg, yield: 95%) as colorless crystals. melting point 94.6-94.8° C.

### Example 466

To a mixed solution of ethyl 5-chloro-6-[2-[3-({[(cyclopropylmethyl)amino]carbonyl}oxy)propyl]-5-(2-methoxyethoxy)phenoxy]nicotinate (297 mg) in tetrahydrofuran (3 ml) and ethanol (3 ml) was added a 1N aqueous sodium hydroxide solution (1.2 ml), and the mixture was stirred at 60° C. for 1 hr. After cooling, the reaction mixture was concentrated, and the concentrate was neutralized with 1N hydrochloric acid. Water was poured into the obtained mixture, and the mixture was extracted with ethyl acetate. The ethyl acetate layer was washed with saturated brine, dried (MgSO₄), and concentrated. The obtained crude crystals were recrystallized from ethyl acetate-hexane to give (2Z)-3-[2-{[3-chloro-5-(trifluoromethyl)pyridin-2-yl]oxy}-4-(3-methoxypropoxy)phenyl]-2-methoxyacrylic acid (270 mg, yield: 96%) as colorless crystals. melting point 139.5-140.8° C.

### Example 467

To a solution of (2E)-3-[2-[(5-bromo-3-methylpyridin-2-yl)oxy]-4-(2-methoxyethoxy)phenyl]-N-(pentylsulfonyl)acrylamide (310 mg) in 1,2-dimethoxyethane (10 ml) were added dihydroxyphenylborane (109 mg), a 2N sodium carbonate aqueous solution (1.2 ml) and tetrakis(triphenylphosphine)palladium (0) (35 mg), and the mixture was heated under reflux for 24 hr. After cooling, water was poured into the reaction mixture, and the mixture was extracted with ethyl acetate. The ethyl acetate layer was washed with saturated brine, dried (MgSO₄), and concentrated. The obtained residue was subjected to silica gel column chromatography, and eluted with ethyl acetate-hexane (2:3, v/v). The obtained crude crystals were recrystallized from ethyl acetate-hexane to give (2E)-3-{4-(2-methoxyethoxy)-2-[(3-methyl-5-phenylpyridin-2-yl)oxy]phenyl}-N-(pentylsulfonyl)acrylamide (205 mg, yield: 65%) as colorless crystals. melting point 132.8-133.5° C.

### Example 468

To a solution of 3-[2-{[3-chloro-5-(trifluoromethyl)pyridin-2-yl]oxy}-4-(2-hydroxy-2-methylpropoxy)phenyl]propan-1-ol (0.30 g) in acetonitrile (5 ml) was added chlorosulfonylisocyanate (0.11 g) under ice-cooling, and the mixture was stirred for 30 min. Pyridine (0.21 ml) was added to the reaction mixture, and the mixture was stirred for 1 hr. (2-Aminoethyl)isopropyl ether (0.38 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-7:3, v/v) and concentrated to give 3-[2-{[3-chloro-5-(trifluoromethyl)pyridin-2-yl]oxy}-4-(2-hydroxy-2-methylpropoxy)phenyl]propyl {[(2-isopropoxyethyl)amino]sulfonyl}carbamate (0.008 g, 1%) as a colorless solid.

¹H-NMR (300 MHz, CDCl₃) δ:1.06-1.17 (6H, m), 1.33 (3H, s), 1.55 (3H, s), 1.87-2.01 (2H, m), 2.57 (2H, t, J=7.3 Hz), 3.13-3.28 (2, H, m), 3.43-3.65 (3H, m), 3.77 (1H, s), 4.02-4.19 (3H, m), 4.54 (1H, br.s.), 5.31-5.51 (1H, m), 6.69 (1H, t, J=2.6 Hz), 6.76-6.87 (1H, m), 7.12-7.23 (1H, m), 7.42-7.65 (1H, m), 8.01 (1H, s), 8.28 (1H, s).

### Example 469

To a solution of 3-[2-{[3-chloro-5-(trifluoromethyl)pyridin-2-yl]oxy}-4-(2-hydroxy-2-methylpropoxy)phenyl]propan-1-ol (0.30 g) in acetonitrile (5 ml) was added chlorosulfonylisocyanate (0.11 g) under ice-cooling, and the mixture was stirred for 30 min. Pyridine (0.21 ml) was added to the reaction mixture, and the mixture was stirred for 1 hr. 2-(Aminoethyl)pyridine (0.45 g) was added, and the mixture was stirred while warming to room temperature over 12 hr. 1N Hydrochloric acid was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-7:3, v/v), concentrated, and crystallized from ethyl acetate-hexane to give 3-[2-{[3-chloro-5-(trifluoromethyl)pyridin-2-yl]oxy}-4-(2-hydroxy-2-methylpropoxy)phenyl]propyl {[(2-pyridin-2-ylethyl)amino]sulfonyl}carbamate (0.043 g, 9%) as colorless crystals.

¹H-NMR (300 MHz, CDCl₃) δ:1.54 (6H, s), 1.78-2.00 (2H, m), 2.43-2.61 (2H, m), 2.93-3.12 (2H, m), 3.40-3.60 (2H, m), 3.93-4.19 (4H, m), 4.37-4.62 (2H, m), 6.42 (1H, br.s.), 6.66 (1H, dd, J=11.7, 2.4 Hz), 6.71-6.86 (1H, m), 7.01-7.22 (3H, m), 7.52-7.67 (1H, m), 7.99 (1H, t, J=2.5 Hz), 8.21-8.31 (1H, m), 8.44-8.54 (1H, m).

### Example 470

(2E)-3-(2-{[3-Chloro-5-(trifluoromethyl)pyridin-2-yl]oxy}-4-isopropoxyphenyl)-2-methylacrylic acid (0.30 g), N-[(5-methylpyrazin-2-yl)methyl]sulfamide (0.16 g), 1-ethyl-3-(3-dimethylaminopropyl)carbodiimide hydrochloride (0.17 g), 4-dimethylaminopyridine (0.11 g) and N,N-dimethylformamide (5 ml) were mixed, and the mixture was stirred at room temperature for 12 hr. Water was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (2:8-1:0, v/v), concentrated, and crystallized from ethyl acetate-hexane to give (2E)-3-(2-{[3-chloro-5-(trifluoromethyl)pyridin-2-yl]oxy}-4-isopropoxyphenyl)-2-methyl-N-({[(5-methylpyrazin-2-yl)methyl]amino}sulfonyl)acrylamide (0.030 g, 7%) as colorless crystals. melting point 126-128° C.

### Example 471

(2E)-3-(2-{[3-Chloro-5-(trifluoromethyl)pyridin-2-yl]oxy}-4-isopropoxyphenyl)-2-methylacrylic acid (0.30 g), N-(2-pyridin-2-ylethyl)sulfamide (0.16 g), 1-ethyl-3-(3-dimethylaminopropyl)carbodiimide hydrochloride (0.17 g), 4-dimethylaminopyridine (0.11 g) and N,N-dimethylformamide (5 ml) were mixed, and the mixture was stirred at room temperature for 12 hr. Water was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-7:3, v/v), concentrated, and crystallized from ethyl acetate-hexane to give (2E)-3-(2-{[3-chloro-5-(trifluoromethyl)pyridin-2-yl]oxy}-4-isopropoxyphenyl)-2-methyl-N-{[(2-pyridin-2-ylethyl)amino]sulfonyl}acrylamide (0.077 g, 18%) as colorless crystals. melting point 121-123° C.

### Example 472

(2E)-3-(2-{[3-Chloro-5-(trifluoromethyl)pyridin-2-yl]oxy}-4-isopropoxyphenyl)-2-methylacrylic acid (0.30 g), pyrrolidine-1-sulfonamide (0.12 g), 1-ethyl-3-(3-dimethylaminopropyl)carbodiimide hydrochloride (0.17 g), 4-dimethylaminopyridine (0.11 g) and N,N-dimethylformamide (5 ml) were mixed, and the mixture was stirred at room temperature for 12 hr. Water was added to the reaction mixture, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-1:1, v/v), and concentrated to give (2E)-3-(2-{[3-chloro-5-(trifluoromethyl)pyridin-2-yl]oxy}-4-isopropoxyphenyl)-2-methyl-N-(pyrrolidin-1-ylsulfonyl)acrylamide (0.10 g, 26%) as a colorless amorphous solid.

¹H-NMR (300 MHz, CDCl₃) δ:1.36 (6H, d, J=6.0 Hz), 1.89 (4H, br.s.), 1.99-2.12 (3H, m), 3.49 (4H, br.s.), 4.45-4.64 (1H, m), 6.71 (1H, d, J=2.3 Hz), 6.84 (1H, dd, J=8.7, 2.4 Hz), 7.18-7.38 (2H, m), 7.86-8.04 (2H, m), 8.25 (1H, d, J=1.1 Hz).

### Example 473

To a solution of 3-(4-butoxy-2-{[3-chloro-5-(trifluoromethyl)pyridin-2-yl]oxy}phenyl)propan-1-ol (465 mg) in toluene (12 ml) was added chlorosulfonylisocyanate (171 mg) under ice-cooling, and the mixture was stirred for 30 min. Pyridine (273 mg) was added to the reaction mixture and the mixture was stirred for 1 hr. 2-Aminoethyl isopropyl ether (713 mg) was added, and the mixture was stirred at room temperature for 2.5 hr. Water was added to the reaction mixture and the mixture was extracted with ethyl acetate. The organic layer was washed with brine, dried (MgSO₄), filtrated and concentrated. The obtained residue was subjected to silica gel column chromatography, and eluted with ethyl acetate-hexane (1:3-1:1, v/v) to give colorless crystals. Recrystallization from ethyl acetate-hexane gave 3-(4-butoxy-2-{[3-chloro-5-(trifluoromethyl)pyridin-2-yl]oxy}phenyl)propyl {[(2-isopropoxyethyl)amino]sulfonyl}carbamate (430 mg, yield: 61%) as colorless crystals. melting point 91.4-91.6° C.

### Example 474

To a solution of 3-[2-{[3-chloro-5-(trifluoromethyl)pyridin-2-yl]oxy}-4-(3-methoxypropoxy)phenyl]propan-1-ol (452 mg) in toluene (11 ml) was added chlorosulfonylisocyanate (160 mg) under ice-cooling, and the mixture was stirred for 30 min. Pyridine (255 mg) was added to the reaction mixture and the mixture was stirred for 1 hr. 2-Aminoethyl isopropyl ether (667 mg) was added, and the mixture was stirred at room temperature for 3 hr. Water was added to the reaction mixture and the mixture was extracted with ethyl acetate. The organic layer was washed with brine, dried (MgSO₄), filtrated and concentrated. The obtained residue was subjected to silica gel column chromatography, and eluted with ethyl acetate-hexane (1:3-1:1, v/v) to give colorless crystals. Recrystallization from ethyl acetate-hexane gave 3-[2-{[3-chloro-5-(trifluoromethyl)pyridin-2-yl]oxy}-4-(3-methoxypropoxy)phenyl]propyl {[(2-isopropoxyethyl)amino]sulfonyl}carbamate (298 mg, yield: 44%) as colorless crystals. melting point 67.4-68.0° C.

### Example 475

To a solution of 3-(4-(but-3-en-1-yloxy)-2-{[3-chloro-5-(trifluoromethyl)pyridin-2-yl]oxy}phenyl)propan-1-ol (402 mg) in toluene (10 ml) was added chlorosulfonylisocyanate (148 mg) under ice-cooling, and the mixture was stirred for 30 min. Pyridine (237 mg) was added to the reaction mixture, and the mixture was stirred for 1 hr. 2-Aminoethyl isopropyl ether (619 mg) was added, and the mixture was stirred at room temperature for 18 hr. Water was added to the reaction mixture and the mixture was extracted with ethyl acetate. The organic layer was washed with brine, dried (MgSO₄), filtrated and concentrated. The obtained residue was subjected to silica gel column chromatography, and eluted with ethyl acetate-hexane (3:7-2:3, v/v) to give colorless crystals. Recrystallization from ethyl acetate-hexane gave 3-(4-(but-3-en-1-yloxy)-2-{[3-chloro-5-(trifluoromethyl)pyridin-2-yl]oxy}phenyl)propyl {[(2-isopropoxyethyl)amino]sulfonyl}carbamate (399 mg, yield: 65%) as colorless crystals. melting point 111.1-113.0° C.

### Example 476

To a solution of 3-[2-{[3-chloro-5-(trifluoromethyl)pyridin-2-yl]oxy}-4-(2-furylmethoxy)phenyl]propan-1-ol (424 mg) in toluene (10 ml) was added chlorosulfonylisocyanate (147 mg) under ice-cooling, and the mixture was stirred for 30 min. Pyridine (235 mg) was added to the reaction mixture, and the mixture was stirred for 1 hr. 2-Aminoethyl isopropyl ether (614 mg) was added, and the mixture was stirred at room temperature for 4 hr. Water was added to the reaction mixture and the mixture was extracted with ethyl acetate. The organic layer was washed with brine, dried (MgSO₄), filtrated and concentrated. The obtained residue was subjected to silica gel column chromatography, and eluted with ethyl acetate-hexane (3:7-1:1, v/v) to give colorless crystals. Recrystallization from ethyl acetate-hexane gave 3-[2-{[3-chloro-5-(trifluoromethyl)pyridin-2-yl]oxy}-4-(2-furylmethoxy)phenyl]propyl {[(2-isopropoxyethyl)amino]sulfonyl}carbamate (359 mg, yield: 57%) as colorless crystals. melting point 118.0-119.8° C.

### Example 477

To a solution of 3-[2-{[3-chloro-5-(trifluoromethyl)pyridin-2-yl]oxy}-4-(2-morpholin-4-ylethoxy)phenyl]propan-1-ol (600 mg) in acetonitrile (13 ml) was added chlorosulfonylisocyanate (194 mg) under ice-cooling, and the mixture was stirred for 30 min. Pyridine (309 mg) was added to the reaction mixture, and the mixture was stirred for 1 hr. 2-Aminoethyl isopropyl ether (807 mg) was added, and the mixture was stirred at room temperature for 60 hr. Water was added to the reaction mixture and the mixture was extracted with ethyl acetate. The organic layer was washed with brine, dried (MgSO₄), filtrated and concentrated. The obtained residue was subjected to silica gel column chromatography, and eluted with ethyl acetate-hexane (1:1-9:1, v/v) to give colorless crystals. Recrystallization from ethyl acetate-hexane gave 3-[2-{[3-chloro-5-(trifluoromethyl)pyridin-2-yl]oxy}-4-(2-morpholin-4-ylethoxy)phenyl]propyl {[(2-isopropoxyethyl)amino]sulfonyl}carbamate (376 mg, yield: 43%) as colorless crystals.

¹H-NMR (300 MHz, CDCl₃) δ:1.13 (6H, d, J=6.0 Hz), 1.86-2.07 (2H, m), 2.49-2.63 (6H, m), 2.80 (2H, t, J=5.6 Hz), 3.17-3.26 (2H, m), 3.48-3.62 (3H, m), 3.69-3.76 (4H, m), 4.05-4.19 (4H, m), 5.43 (1H, br. s.), 6.64-6.68 (1H, m), 6.77-6.82 (1H, m), 7.17 (1H, d, J=8.7 Hz), 8.01 (1H, d, J=1.7 Hz), 8.25-8.29 (1H, m).

### Example 478

To a solution of 3-[2-{[3-chloro-5-(trifluoromethyl)pyridin-2-yl]oxy}-4-(tetrahydrofuran-2-ylmethoxy)phenyl]propan-1-ol (383 mg) in toluene (8.9 ml) was added chlorosulfonylisocyanate (132 mg) under ice-cooling, and the mixture was stirred for 30 min. Pyridine (210 mg) was added to the reaction mixture, and the mixture was stirred for 1 hr. 2-Aminoethyl isopropyl ether (549 mg) was added, and the mixture was stirred at room temperature for 60 hr. Water was added to the reaction mixture and the mixture was extracted with ethyl acetate. The organic layer was washed with brine, dried (MgSO₄), filtrated and concentrated. The obtained residue was subjected to silica gel column chromatography, and eluted with ethyl acetate-hexane (7:13-7:3, v/v) to give colorless crystals. Recrystallization from ethyl acetate-hexane gave 3-[2-{[3-chloro-5-(trifluoromethyl)pyridin-2-yl]oxy}-4-(tetrahydrofuran-2-ylmethoxy)phenyl]propyl {[(2-isopropoxyethyl)amino]sulfonyl}carbamate (246 mg, yield: 45%) as colorless crystals.

¹H-NMR (300 MHz, CDCl₃) δ:1.13 (6H, d, J=6.0 Hz), 1.68-1.83 (2H, m), 1.86-2.12 (4H, m), 2.55 (2H, t, J=7.3 Hz), 3.23 (2H, d, J=4.3 Hz), 3.44-3.62 (3H, m), 3.76-3.96 (4H, m), 4.14 (2H, t, J=6.4 Hz), 4.21-4.30 (1H, m), 5.43 (1H, d, J=1.7 Hz), 6.67 (1H, d, J=2.4 Hz), 6.82 (1H, dd, J=8.5, 2.6 Hz), 7.18 (1H, d, J=8.5 Hz), 8.01 (1H, d, J=2.3 Hz), 8.27 (1H, dd, J=2.2, 1.0 Hz).

### Example 479

To a solution of 3-(2-{[3-chloro-5-(trifluoromethyl)pyridin-2-yl]oxy}-4-{2-[(triisopropylsilyl)oxy]ethoxy}phenyl)propan-1-ol (10.9 g) in acetonitrile (150 ml) was added chlorosulfonylisocyanate (2.95 g) under ice-cooling, and the mixture was stirred for 30 min. Pyridine (5.9 ml) was added to the reaction mixture, and the mixture was stirred for 1 hr. (2-Aminoethyl)isopropyl ether (10.8 g) was added, and the mixture was stirred while warming to room temperature over 6 hr. A saturated aqueous sodium hydrogencarbonate solution was added to the reaction mixture, and the mixture was concentrated. Water was added to the residue, and the mixture was extracted with ethyl acetate. The ethyl acetate layer was washed with saturated brine, dried (MgSO₄), and concentrated.

To a solution of the residue in tetrahydrofuran (100 ml) was added a 1.0M tetra-n-butylammonium fluoride tetrahydrofuran solution (100 ml), and the mixture was stirred at room temperature for 3 hr. The reaction mixture was concentrated, water was added to the residue, and the mixture was extracted with ethyl acetate. The ethyl acetate layer was washed with saturated brine, dried (MgSO₄), and concentrated. The residue was subjected to silica gel column chromatography, eluted with ethyl acetate-hexane (0:1-2:8, v/v), concentrated and crystallized from ethyl acetate-hexane. The obtained crystals were subjected to HPLC (acetonitrile:water, containing 0.01% TFA, 5:95-100:0 v/v) and further crystallized from ethyl acetate-hexane to give 3-(4-{2-[(aminosulfonyl)oxy]ethoxy}-2-{[3-chloro-5-(trifluoromethyl)pyridin-2-yl]oxy}phenyl)propyl {[(2-isopropoxyethyl)amino]sulfonyl}carbamate (0.18 g, 1%) as colorless crystals.

¹H-NMR (300 MHz, CDCl₃) δ:1.14 (6H, d, J=6.1 Hz), 1.87-2.02 (2H, m), 2.61 (2H, t, J=7.0 Hz), 3.16-3.27 (2H, m), 3.48-3.64 (3H, m), 4.11-4.18 (2H, m), 4.23-4.32 (2H, m), 4.52 (2H, dd, J=5.3, 3.4 Hz), 4.98 (2H, br.s.), 5.38 (1H, t, J=5.7 Hz), 6.71 (1H, d, J=2.7 Hz), 6.82 (1H, dd, J 8.7, 2.7 Hz), 7.22 (1H, d, J=8.7 Hz), 7.77 (1H, s), 8.03 (1H, d, J=2.3 Hz), 8.28 (1H, s).

### Experimental Example 1

### Blood Glucose and Blood Lipid (Triglyceride) Lowering Action in Mice

The test compound was mixed with a powder diet (CE-2, CLEA JAPAN, INC.) at a proportion of 0.01% (0.03% for the compounds of Examples 1, 2, 12, 81, 94, 96, 129 and 143, and 0.005% for the compounds of Examples 28, 30 and 37) and freely given to KKAy mice (9 to 14-week-old, 5 mice per group) which are obesity non-insulin-dependent diabetes (type-2 diabetes) models, for 4 to 7 days. During this period, water was freely given. The blood was drawn from the orbital venous plexus. Glucose and triglyceride in the plasma separated from the blood were quantitated using L Type Wako Glu2 (Wako Pure Chemical Industries, Ltd.) and L Type Wako TG•H (Wako Pure Chemical Industries, Ltd.), respectively, according to an enzyme method. The results are shown in Table 1.

In the Table, the “hypoglycemic action (%)” shows a relative value (%) of the blood glucose level of the test compound administration group when the blood glucose level of the test compound non-administration group is 100%. In addition, the “hypolipidemic action (%)” shows a relative value (%) of the blood triglyceride of the test compound administration group when the blood triglyceride value of the test compound non-administration group is 100%.

TABLE 1

| test compound | hypoglycemic action | hypolipidemic action |
|---|---|---|
| (Example No.) | (%) | (%) |
| 1 | 47 | 53 |
| 2 | 50 | 41 |
| 12 | 46 | 43 |
| 26 | 50 | 62 |
| 28 | 52 | 32 |
| 30 | 47 | 57 |
| 37 | 68 | 70 |
| 58 | 64 | 73 |
| 81 | 44 | 60 |
| 94 | 48 | 79 |
| 96 | 53 | 57 |
| 97 | 58 | 64 |
| 112 | 53 | 70 |
| 129 | 45 | 76 |
| 143 | 47 | 76 |
| 151 | 59 | 73 |
| 155 | 70 | 70 |
| 183 | 48 | 44 |
| 196 | 54 | 79 |
| 197 | 58 | 87 |
| 198 | 62 | 84 |
| 204 | 52 | 36 |
| 205 | 54 | 50 |
| 207 | 54 | 43 |
| 208 | 70 | 71 |
| 210 | 58 | 69 |
| 211 | 49 | 46 |
| 214 | 56 | 65 |
| 218 | 81 | 129 |
| 250 | 78 | 121 |
| 252 | 46 | 20 |
| 424 | 70 | 18 |
| 429 | 51 | 60 |
| 430 | 45 | 34 |
| 437 | 45 | 58 |
| 439 | 49 | 34 |
| 444 | 55 | 52 |

As shown above, it is clear that the compound of the present invention has superior hypoglycemic action and hypolipidemic action, and is useful as an agent for the prophylaxis or treatment of diabetes, hyperlipidemia (particularly, hypertriglyceridemia), impaired glucose tolerance and the like.

### Experimental Example 2

### PPARγ-RXRα Heterodimer Ligand Activity

The PPARγ:RXRα:4ERPP/CHO-K1 cells described in WO03/099793 were cultured in a Ham F12 medium [manufactured by Life Technologies, Inc., US] containing 10% calf fetal serum [manufactured by Life Technologies, Inc., US], sown in a 96-well white plate [manufactured by Corning Coster Corporation, US] at 1×10⁴ cells/well, and incubated overnight in a carbon dioxide gas incubator at 37° C.

Then, the medium was removed from the 96 well white plate, 45 μl of Ham F12 medium containing 0.1% fatty acid-free bovine serum albumin (BSA) and a test compound (5 μl) were added, and the cells were incubated for one day in a carbon dioxide gas incubator at 37° C. The medium was removed, 20 μl of PicaGene 7.5 (manufactured by Wako Pure Chemical Industries, Ltd.) 2-fold diluted with HBSS (HANKS' BALANCED SALT SOLUTION) [manufactured by BIO WHITTAKER, US] was added. After stirring, the luciferase activity was determined using the 1420 ARVO Multilabel Counter [manufactured by PerkinElmer, US].

The induction rate was calculated from the luciferase activity of each test compound based on the luciferase activity of the test compound non-administration group as 1. The test compound concentration and the induction rate were analyzed by PRISM [manufactured by GraphPad Software, Inc., US] to calculate EC₅₀ value (compound concentration showing 50% of the maximum value of induction rate) of the test compound. The results are shown in Table 2.

TABLE 2

| test compound | EC₅₀ |
|---|---|
| (Example No.) | (nM) |
| 1 | 1.5 |
| 2 | 2.6 |
| 12 | 15 |
| 13 | 26 |
| 26 | 4.6 |
| 28 | 4.5 |
| 30 | 3.8 |
| 32 | 6.6 |
| 37 | 21 |
| 44 | 6.9 |
| 47 | 3.4 |
| 51 | 20 |
| 58 | 10 |
| 64 | 8.7 |
| 81 | 16 |
| 94 | 20 |
| 96 | 8.4 |
| 97 | 7.5 |
| 111 | 9.3 |
| 112 | 8.3 |
| 129 | 10 |
| 151 | 7.2 |
| 155 | 12 |
| 173 | 9.8 |
| 183 | 3.8 |
| 190 | 15 |
| 196 | 4.4 |
| 197 | 28 |
| 198 | 7.8 |
| 199 | 13 |
| 204 | 0.92 |
| 205 | 3.1 |
| 207 | 0.99 |
| 208 | 4.0 |
| 209 | 2.3 |
| 210 | 6.7 |
| 211 | 5.2 |
| 214 | 1.1 |
| 218 | 80 |
| 223 | 55 |
| 224 | 16 |
| 225 | 16 |
| 226 | 8.5 |
| 248 | 18 |
| 250 | 7.2 |
| 251 | 7.4 |
| 252 | 3.1 |
| 255 | 3.9 |
| 256 | 13 |
| 257 | 2.1 |
| 258 | 1.1 |
| 371 | 4.7 |
| 424 | 6.2 |
| 427 | 22 |
| 429 | 3.8 |
| 430 | 0.95 |
| 437 | 54 |
| 439 | 1.8 |
| 444 | 3.4 |
| 446 | 13 |
| 451 | 15 |
| 453 | 7.3 |

As shown above, it is clear that the compound of the present invention has superior PPARγ-RXRα heterodimer ligand activity.

### Formulation Example 1

### Production of Capsule

| | |
|---|---|
| 1) compound of Example 1 | 30 mg |
| 2) finely divided powder cellulose | 10 mg |
| 3) lactose | 19 mg |
| 4) magnesium stearate | 1 mg |
| total | 60 mg |

1), 2), 3) and 4) are mixed and filled in a gelatin capsule.

### Formulation Example 2

### Production of Tablet

| | |
|---|---|
| 1) compound of Example 1 | 30 g |
| 2) lactose | 50 g |
| 3) cornstarch | 15 g |
| 4) calcium carboxymethylcellulose | 44 g |
| 5) magnesium stearate | 1 g |
| 1000 tablets total | 140 g |

The total amount of 1), 2), 3) and 30 g of 4) are kneaded with water, vacuum dried and sized. The sized powder is mixed with 14 g of 4) and 1 g of 5) and the mixture is punched out with a tableting machine. In this way, 1000 tablets containing 30 mg of the compound of Example 1 per tablet are obtained.

### INDUSTRIAL APPLICABILITY

The compound of the present invention is useful as an agent for the prophylaxis or treatment of diabetes, which is associated with a fewer side effects such as body weight gain, adipocyte accumulation, cardiac hypertrophy and the like.

This application is based on a patent application No. 2005-232646 filed in Japan, the contents of which are incorporated in full herein by this reference.

1-2. (canceled)

3. The method of claim 18, wherein Ar is an optionally substituted monocyclic aromatic ring.

4. A compound represented by the formula: 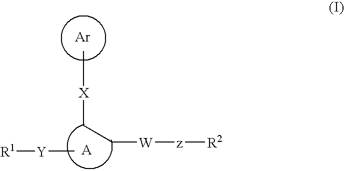
wherein ring A, Ar, R¹, R², X, Y, W, Z are as defined in claim 18 (provided that Ar is not an unsubstituted benzene ring), or a salt thereof, which excludes the following compounds:
(4-(2-{[(4-chlorophenyl)sulfonyl]amino}ethyl)-3-{[3-(quinolin-2-ylmethoxy)benzyl]oxy}phenoxy)acetic acid,
ethyl (4-(2-{[(4-chlorophenyl)sulfonyl]amino}ethyl)-3-{[3-(quinolin-2-ylmethoxy)benzyl]oxy}phenoxy)acetate,
1-{4-methoxy-2-[(4-vinylbenzyl)oxy]phenyl}ethyl[4-(diethylamino)-2-methylphenyl]carbamate,
N-{2-[4,5-dimethoxy-2-(2-thienylcarbonyl)phenyl]ethyl}-N,4-dimethylbenzenesulfonamide,
N-(2,2-dimethoxyethyl)-N-{3-[6-({(2,2-dimethoxyethyl)[(4-methylphenyl)sulfonyl]amino}methyl)-2,3-dimethoxyphenoxy]-4-methoxybenzyl}-4-methylbenzenesulfonamide, and
2-[2-(3,4-dimethoxyphenyl)ethyl]-4,5-dimethoxybenzyl phenylcarbamate.

5. The compound of claim 4, wherein Ar is an optionally substituted monocyclic aromatic ring, or a salt thereof.

6. The compound of claim 4, wherein Ar is an optionally substituted 5- or 6-membered monocyclic aromatic heterocycle, or a salt thereof.

7. The compound of claim 4, wherein Ar is a substituted benzene ring, or a salt thereof.

8. The compound of claim 4, wherein X is an oxygen atom, or a salt thereof.

9. The compound of claim 4, wherein Z is —CONR^{a}SO₂—, or a salt thereof.

10. The compound of claim 4, wherein R¹ is (1) a C₁₋₁₀ alkyl group or a C₂₋₁₀ alkenyl group, each optionally substituted by 1 to 3 substituents selected from
a C₁₋₆ alkoxy group optionally substituted by a C₁₋₆ alkoxy group;
a carbamoyl group optionally mono- or di-substituted by a C₁₋₆ alkyl group;
an aromatic heterocyclic group optionally substituted by a C₁₋₆ alkyl group;
a non-aromatic heterocyclic group optionally substituted by 1 to 3 substituents selected from a C₁₋₆ alkyl group and an oxo group;
a C₁₋₆ alkoxy-carbonyl group;
a carboxyl group;
a hydroxy group;
a cyano group;
a silyloxy group optionally substituted by 1 to 3 substituents selected from a C₁₋₆ alkyl group and a C₆₋₁₄ aryl group;
a C₁₋₆ alkyl-carbonyloxy group;
a C₃₋₁₀ cycloalkyloxy group;
a C₃₋₁₀ cycloalkyl-C₁₋₆ alkyloxy group;
a C₁₋₆ alkylsulfonyl group;
a C₁₋₆ alkyl-carbonyl group; and
a sulfamoyloxy group;
(2) a C₃₋₁₀ cycloalkyl group;
(3) a C₆₋₁₄ aryl group;
(4) a C₇₋₁₃ aralkyl group;
(5) a C₃₋₁₀ cycloalkyl-C₁₋₆ alkyl group;
(6) a monocyclic non-aromatic heterocyclic group; or
(7) a monocyclic aromatic heterocyclic group, or a salt thereof.

11. The compound of claim 4, wherein R² is
(1) a hydrogen atom;
(2) a C110 alkyl group or a C₂₋₁₀ alkenyl group, each optionally substituted by 1 to 3 substituents selected from a C₁₋₆ alkoxy group; a halogen atom; a hydroxy group; a cyano group; a C₁₋₆ alkylthio group; a carbamoyl group; a C₆₋₁₄ aryloxy group; an amino group optionally substituted by 1 or 2 substituents selected from a C₁₋₆ alkyl group, a C₁₋₆ alkyl-carbonyl group and a C₆₋₁₄ aryl group; an aromatic heterocyclic group optionally substituted by 1 to 3 C₁₋₆ alkyl group; and a non-aromatic heterocyclic group optionally substituted by 1 to 3 substituents selected from a C₁₋₆ alkyl group and an oxo group;
(3) a C₃₋₁₀ cycloalkyl group optionally substituted by a C₁₋₆ alkyl group and optionally condensed with a benzene ring;
(4) a C₆₋₁₄ aryl group optionally substituted by 1 to 3 substituents selected from a C₁₋₆ alkyl group optionally substituted by 1 to 3 halogen atoms, a hydroxy group, a C₁₋₆ alkoxy group, a halogen atom, a nitro group, and a cyano group;
(5) a C₇₋₁₃ aralkyl group optionally substituted by 1 to 3 substituents selected from a C₁₋₆ alkoxy group and a C₆₋₁₄ aryl group;
(6) a C₃₋₁₀ cycloalkyl-C₁₋₆ alkyl group; or
(7) a non-aromatic heterocyclic group optionally substituted by an oxo group, or a salt thereof.

12. The compound of claim 4, wherein ring A is a benzene ring or a 5- or 6-membered aromatic heterocycle, or a salt thereof.

13. The compound of claim 4, wherein Y is a bond, —O— or —SO₂—, or a salt thereof.

14. The compound of claim 4, wherein W is C₁₋₆ alkylene or C₂₋₆ alkenylene, or a salt thereof.

15. The compound of claim 4, which is
3-(2-{[3-chloro-5-(trifluoromethyl)pyridin-2-yl]oxy}-4-isopropoxyphenyl)-N-(pentylsulfonyl)propanamide,
(2E)-3-[2-{[3-chloro-5-(trifluoromethyl)pyridin-2-yl]oxy}-4-(2-methoxyethoxy)phenyl]-N-(pentylsulfonyl)acrylamide,
3-[2-{[3-chloro-5-(trifluoromethyl)pyridin-2-yl]oxy}-4-(2-methoxyethoxy)phenyl]propyl (pentylsulfonyl)carbamate,
3-[3-butoxy-1-(2,4-dichlorobenzyl)-1H-pyrazol-5-yl]-N-(pentylsulfonyl)propanamide,
3-{3-tert-butyl-1-[2-chloro-4-(trifluoromethyl)benzyl]-1H-pyrazol-5-yl}-N-(pentylsulfonyl)propanamide,
butyl ({2-[3-butoxy-1-(2,4-dichlorobenzyl)-1H-pyrazol-5-yl]ethyl}sulfonyl)carbamate,
(2E)-3-{2-{[3-chloro-5-(trifluoromethyl)pyridin-2-yl]oxy}-4-[2-ethoxy-1-(ethoxymethyl)ethoxy]phenyl}-N-(pentylsulfonyl)acrylamide,
3-[2-{[3-chloro-5-(trifluoromethyl)pyridin-2-yl]oxy}-4-(2-methoxyethoxy)phenyl]propyl {[(2-isopropoxyethyl)amino]sulfonyl}carbamate,
3-(2-{[3-chloro-5-(trifluoromethyl)pyridin-2-yl]oxy}-4-(2-methoxyethoxy)phenyl]propyl {[(2-pyridin-2-ylethyl)amino]sulfonyl}carbamate,
3-[2-{[3-chloro-5-(trifluoromethyl)pyridin-2-yl]oxy}-4-(2-methoxyethoxy)phenyl]-N—[(pentylamino)sulfonyl]propanamide,
(2E)-3-[2-{[3-chloro-5-(trifluoromethyl)pyridin-2-yl]oxy}-4-(2-hydroxy-2-methylpropoxy)phenyl]-N-(pentylsulfonyl)acrylamide or
3-[2-{[3-chloro-5-(trifluoromethyl)pyridin-2-yl]oxy}-4-(2-hydroxyethoxy)phenyl]propyl {[(2-isopropoxyethyl)amino]sulfonyl}carbamate,
or a salt thereof.

16. A prodrug of the compound of claim 4 or a salt thereof.

17. A pharmaceutical agent comprising the compound of claim 4 or a salt thereof or a prodrug thereof.

18. A method for the prophylaxis or treatment of diabetes in a mammal, which comprises administering a compound represented by the formula: 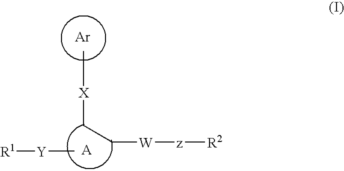
wherein ring A is an aromatic ring which is optionally further substituted;
Ar is an optionally substituted monocyclic ring;
R¹ is an optionally substituted hydrocarbon group or an optionally substituted heterocyclic group;
R² is a hydrogen atom, an optionally substituted hydrocarbon group or an optionally substituted heterocyclic group;
X is a spacer having a main chain of 1 or 2 atoms;
Y is a bond or a spacer having a main chain of 1 or 2 atoms;
W is an optionally substituted divalent hydrocarbon group having 1 to 20 carbon atoms;
Z is —CONR^{a}SO₂—, —SO₂NR^{a}CO—, —SO₂NR^{a}COO—, —NR^{a}SO₂—, —OCONR^{a}SO₂—, —OCONR^{a}SO₂NR^{c}—, —OCONR^{c}—, —NR^{a}CONR^{b}SO₂—, —NR^{a}SO₂NR^{b}COO— or —CONR^{a}SO₂NR^{a}— (R^{a} and R^{b} are each independently a hydrogen atom, an optionally substituted hydrocarbon group or an amino-protecting group, R^{c} is a hydrogen atom, an optionally substituted hydrocarbon group or an amino-protecting group, or R^{c} and R² are bonded to each other to form, together with the adjacent nitrogen atom, an optionally substituted, nitrogen-containing heterocycle),
or a salt thereof or a prodrug thereof to the mammal.

19. A method of improving insulin resistance in a mammal, which comprises administering the compound of claim 18 or a salt thereof or a prodrug thereof to the mammal.

20-21. (canceled)
